(19) United States
(12) Patent Application Publication
(10) Pub. No.: US 2026/0263608 A1
(43) Pub. Date: Sep. 10, 2026

(54) Multi-Albumin Binding Compounds

(71) Applicant: Ascendis Pharma A/S, Hellerup (DK)

(72) Inventors: Kennett SPROGØE, Hellerup (DK); Nicola BISEK, Heidelberg (DE); Daniel STUBBA, Heidelberg (DE); Ulrike REISACHER, Heidelberg (DE); Samuel WEISBROD, Heidelberg (DE)

(21) Appl. No.: 19/159,995

(22) PCT Filed: Mar. 5, 2024
(86) PCT No.: PCT/EP2024/055727

§ 371 Date: Aug. 26, 2025

(30) Foreign Application Priority Data
Mar. 6, 2023 (EP) 23160263.2
Sep. 5, 2023 (EP) 23195403.3
Oct. 16, 2023 (EP) 23203733.3

(51) Int. Cl. A61K 47/60; A61K 47/54; A61K 47/61; A61K 47/64

(52) U.S. Cl. CPC A61K 47/60; A61K 47/542; A61K 47/61; A61K 47/64

(57) ABSTRACT

The present invention relates to a compound or a pharmaceutically acceptable salt thereof, wherein the compound comprises at least two covalently and reversibly conjugated drug moieties, each of the at least two drug moieties comprises an albumin-binding moiety, and wherein the compound releases the drug moieties as their corresponding free drug molecules; to pharmaceutical compositions comprising such compounds and uses thereof.

The present invention relates to a compound or a pharmaceutically acceptable salt thereof, wherein the compound comprises at least two covalently and reversibly conjugated drug moieties, each of the at least two drug moieties comprises an albumin-binding moiety, and wherein the compound releases the drug moieties as their corresponding free drug molecules; to pharmaceutical compositions comprising such compounds and uses thereof.

Whereas some classes of therapeutic proteins, including antibodies, have long inherent half-lives, many other molecules, particularly protein or peptide hormones are often susceptible to enzymatic degradation, renal clearance and/or rapid receptor-mediated clearance, which leads to a short plasma elimination half-life. Furthermore, due to the challenges associated with oral delivery, most peptide- or protein-based drugs require parenteral administration, which combined with a short half-life necessitates frequent injections, resulting in discomfort and inconvenience for the patient. This may negatively impact adherence and, ultimately, limit treatment efficacy and outcomes.

Injecting lipidated peptides has been demonstrated to have slower absorption from the subcutaneous tissue compared to the non-derivatized peptide as well as longer circulatory half-life due to reversible binding to albumin. The half-life of endogenous albumin in the circulation is approximately 3 weeks. This long half-life can be attributed both to its large molecular size, which minimizes renal clearance, as well as to recycling via the neonatal Fc receptor (FcRn).

In the circulation, the large molecular size protects the bound peptide from renal clearance and reduces the rate of distribution to the extravascular compartment.

The slower absorption is thought to be due to a reduced rate of diffusion in the tissue by a mechanism involving interaction of the fatty acid side chain with cell membranes and/or proteins such as albumin present at the injection site. The rate of diffusion in the tissue following injection and the passage over the capillary wall would expectedly be reduced due to the large molecular size of the albumin-peptide complex. Molecules with a molecular size greater than approximately 16 kDa are preferentially alternatively be absorbed via a lymphatic route compared to direct absorption into blood across the capillary wall. Thus, slower absorption and reversible albumin binding has enabled extension of dosing intervals for drugs, such as peptide drugs, to up to a week. However, an even further extension of the dosing intervals would be desirable.

It is an object of the present invention to overcome the shortcomings of current therapies at least partially.

This object is achieved with a compound or a pharmaceutically acceptable salt thereof, wherein the compound comprises at least two covalently and reversibly conjugated drug moieties, each of the at least two drug moieties comprises an albumin-binding moiety, and wherein the compound releases the drug moieties as their corresponding free drug molecules.

Applicant discovered that the compounds of the present invention are capable of further extending half-life and dosing frequency. It was surprisingly found that a compound of two or more drugs comprising an albumin binding moiety may coordinate more than one albumin molecule, resulting in a large molecular size of the albumin-drug complex.

This results in conjugates exhibiting a further increased albumin binding and slowed absorption of the macromolecular drug-albumin complex. Such conjugates also demonstrate a reduced absorption from the site of administration, such as from subcutaneous tissue, compared to administration of the corresponding unconjugated drugs. Consequently, such conjugates have a prolonged half-life compared to the corresponding unconjugated drugs.

Within the present invention the terms are used having the meaning as follows.

As used herein, the term “GLP-1 receptor agonist” refers to agonists of the GLP-1 receptor (GLP1R) and optionally in addition agonists of one or more other receptors, such as for example a receptor for gastric inhibitory polypeptide (GIPR), a receptor for glucagon (GCGR), a receptor for amylin, a receptor for peptide YY (PYYR) or a receptor for glucagon-like peptide-2 (GLP2R). An “agonist” of a receptor, such as an agonist of the GLP-1 receptor, is a chemical compound that activates such receptor to produce a biological response. If a GLP-1 receptor agonist is in addition to being a GLP1R agonist also an agonist of one receptor other than GLP1R, such as an agonist of GIPR, GCGR, an amylin receptor, a PYYR or GLP2R, such GLP-1 receptor agonist is also referred to as being a “dual GLP-1 receptor agonist” or short “dual agonist”. Likewise, if a GLP-1 receptor agonist is in addition to being a GLP1R agonist also an agonist of two other receptors, which may be selected from the group consisting of GIPR, GCGR, an amylin receptor, a PYYR or GLP2R, such GLP-1 receptor agonist is also referred to as being a “triple GLP-1 receptor agonist” or short “triple agonist”.

As used herein, the term “peptide” as used herein refers to a chain of at least 2 and up to and including 50 amino acid monomer moieties, which may also be referred to as “amino acid residues”, linked by peptide (amide) linkages. The amino acid monomers may be selected from the group consisting of proteinogenic amino acids and non-proteinogenic amino acids and may be D- or L-amino acids. The term “peptide” also includes peptidomimetics, such as peptoids, beta-peptides, cyclic peptides and depsipeptides and covers such peptidomimetic chains with up to and including 50 monomer moieties. The cyclic peptides may be mono-, bi-, tri- or tetracyclic peptides. The term “peptide” also includes lasso peptides.

As used herein, the term “protein” refers to a chain of more than 50 amino acid monomer moieties, which may also be referred to as “amino acid residues”, linked by peptide linkages, in which preferably no more than 12000 amino acid monomers are linked by peptide linkages, such as no more than 10000 amino acid monomer moieties, no more than 8000 amino acid monomer moieties, no more than 5000 amino acid monomer moieties or no more than 2000 amino acid monomer moieties.

As used herein, the term “small molecule drug” refers to drugs that are organic compounds with a molecular weight of less than 1000 Da, such as less than 900 Da or less than 800 Da. It is understood that nucleobase-based drug moieties, such as adenine or guanine analogues, may also be a type of small molecule drugs.

As used herein, the terms “medium molecule drug” or “medium size molecule drug” refer to drugs that are organic compounds which are not peptides and which are not proteins and have a molecular weight ranging from and including 1 kDa to 7.5 kDa.

As used herein, the term “oligonucleotide” refers to double- or single-stranded RNA and DNA with preferably 2 to 1000 nucleotides and any modifications thereof. Modifications include, for example, those which provide other chemical groups that incorporate additional charge, polarizability, hydrogen bonding, electrostatic interaction, and fluxionality to the nucleic acid ligand bases or to the nucleic acid ligand as a whole. Such modifications include for example, to 2′-position sugar modifications, 5-position pyrimidine modifications, 8-position purine modifications, modifications at exocyclic amines, substitution of 4-thiouridines, substitution of 5-bromo or 5-iodo-uracil; backbone modifications, methylations, unusual base-pairing combinations such as the isobases isocytidine and isoguanidine. Modifications can also include 3′ and 5′ modifications such as capping and change of stereochemistry. The term also includes aptamers.

As used herein, the term “peptide nucleic acids” refers to organic polymers having a peptidic backbone, i.e., a backbone in which the monomers are connected to each other through peptide linkages, to which nucleobases such as adenine, cytosine, guanine, thymine and uracil, are attached. In certain embodiments, the peptide backbone comprises N-(2-aminoethyl)-glycine.

As used herein, the term “random coil” relates to any conformation of a polymeric molecule, including proteins, in which the individual monomeric elements that form said polymeric structure are essentially randomly oriented towards the adjacent monomeric elements while still being chemically bound to said adjacent monomeric elements. In particular, a polypeptide or protein having random coil conformation substantially lacks a defined secondary and tertiary structure. The nature of polypeptide random coils and their methods of experimental identification are known to the person skilled in the art. In particular, the lack of secondary and tertiary structure of a protein may be determined by circular dichroism (CD) measurements. CD spectroscopy represents a light absorption spectroscopy method in which the difference in absorbance of right- and left-circularly polarized light by a substance is measured. The secondary structure of a protein can be determined by CD spectroscopy using far-ultraviolet spectra with a wavelength between approximately 190 and 250 nm. At these wavelengths the different secondary structures commonly found in conformations each give rise to a characteristic shape and magnitude of the CD spectrum. Accordingly, by using CD spectrometry the skilled artisan is readily capable of determining whether an amino acid polymer adopts random coil conformation at physiological conditions.

When determining whether a peptide or protein adopts random coil conformation under experimental conditions using the methods as described herein, the biophysical parameters such as temperature, pH, osmolarity and protein content may be different to the physiological conditions normally found in vivo. Temperatures between 1° C. and 42° C. or preferably 4° C. to 25° C. may be considered useful to test and/or verify the biophysical properties and biological activity of a peptide or protein under physiological conditions in vitro.

Several buffers, in particular in experimental settings (for example in the determination of protein structures, in particular in circular dichroism (CD) measurements and other methods that allow the person skilled in the art to determine the structural properties of a protein/polypeptide or peptide stretch) or in buffers, solvents and/or excipients for pharmaceutical compositions, are considered to represent “physiological solutions” or “physiological conditions” in vitro. Examples of such buffers are, e.g. phosphate-buffered saline (PBS: 115 mM NaCl, 4 mM KH₂PO₄, 16 mM Na₂HPO₄ pH 7.4), Tris buffers, acetate buffers, citrate buffers or similar buffers such as those used in the appended examples. Generally, the pH of a buffer representing physiological conditions should lie in a range from 6.5 to 8.5, preferably in a range from 7.0 to 8.0, most preferably in a range from 7.2 to 7.7 and the osmolarity should lie in a range from 10 to 1000 mmol/kg H₂O, more preferably in a range from 50 to 500 mmol/kg H₂O and most preferably in a range from 200 to 350 mmol/kg H₂O. Optionally, the protein content of a buffer representing physiological conditions may lie in a range from 0 to 100 g/l, neglecting the protein with biological activity itself, whereby typical stabilizing proteins may be used, for example human or bovine serum albumin.

Other established biophysical methods for determining random coil conformation include nuclear magnetic resonance (NMR) spectroscopy, absorption spectrometry, infrared and Raman spectroscopy, measurement of the hydrodynamic volume via size exclusion chromatography, analytical ultracentrifugation and dynamic/static light scattering as well as measurements of the frictional coefficient or intrinsic viscosity.

As used herein the term “physiological conditions” refers to aqueous buffer at pH 7.4, 37° C.

As used herein the term “pharmaceutical composition” refers to a composition containing one or more active ingredients, such as for example at least one conjugate of the present invention, and one or more excipients, as well as any product which results, directly or indirectly, from combination, complexation or aggregation of any two or more of the ingredients of the composition, or from dissociation of one or more of the ingredients, or from other types of reactions or interactions of one or more of the ingredients. Accordingly, a pharmaceutical composition of the present invention encompasses any composition made by admixing one or more conjugate of the present invention and one or more pharmaceutically acceptable excipient.

As used herein, the term “excipient” refers to a diluent, adjuvant, or vehicle with which the therapeutic, such as a drug or prodrug, is administered. Such pharmaceutical excipient may be sterile liquids, such as water and oils, including those of petroleum, animal, vegetable or synthetic origin, including but not limited to peanut oil, soybean oil, mineral oil, sesame oil and the like. Water is an example for an excipient when the pharmaceutical composition is administered orally. Saline and aqueous dextrose are examples of excipients when the pharmaceutical composition is administered intravenously. Saline solutions and aqueous dextrose and glycerol solutions are in certain embodiments employed as liquid excipients for injectable solutions. Suitable pharmaceutical excipients include starch, glucose, lactose, sucrose, mannitol, trehalose, gelatin, malt, rice, flour, chalk, silica gel, sodium stearate, glycerol monostearate, talc, sodium chloride, dried skim milk, glycerol, propylene, glycol, water, ethanol and the like. The pharmaceutical composition, if desired, can also contain minor amounts of wetting or emulsifying agents, pH buffering agents, like, for example, acetate, succinate, tris, carbonate, phosphate, HEPES (4-(2-hydroxyethyl)-1-piperazineethanesulfonic acid), MES (2-(N-morpholino)ethanesulfonic acid), or can contain detergents, like Tween, poloxamers, poloxamines, CHAPS, Igepal, or amino acids like, for example, glycine, lysine, or histidine. These pharmaceutical compositions can take the form of solutions, suspensions, emulsions, tablets, pills, capsules, powders, or sustained-release formulations. The pharmaceutical composition may be formulated as a suppository, with traditional binders and excipients such as triglycerides. Oral formulation can include standard excipients such as pharmaceutical grades of mannitol, lactose, starch, magnesium stearate, sodium saccharine, cellulose, magnesium carbonate, etc. Such compositions will contain a therapeutically effective amount of the drug or drug moiety, together with a suitable amount of excipient so as to provide the form for proper administration to the patient. The formulation should suit the mode of administration.

As used herein the term “liquid composition” refers to a mixture comprising a water-soluble compound and one or more solvents, such as water.

The term “suspension composition” relates to a mixture comprising at least one water-insoluble compound and one or more solvents, such as water.

As used herein, the term “dry composition” means that a pharmaceutical composition is provided in a dry form. Suitable methods for drying are spray-drying and lyophilization, i.e., freeze-drying. Such dry composition has a residual water content of a maximum of 10%, such as less than 5% or less than 2%, determined according to Karl Fischer. In certain embodiments such dry pharmaceutical composition is dried by lyophilization.

The term “drug” as used herein refers to a substance used in the treatment, cure, prevention, or diagnosis of a disease or used to otherwise enhance physical or mental well-being. If a drug is conjugated to another moiety, the moiety of the resulting product that originated from the drug is referred to as “drug moiety”.

As used herein the term “prodrug” refers to a covalent conjugate in which a drug moiety is reversibly and covalently connected to a specialized protective group through a reversible linker moiety, also referred to as “reversible prodrug linker moiety” or “reversible linker moiety”, which is conjugated through a reversible linkage to the drug moiety and wherein the specialized protective group alters or eliminates undesirable properties in the parent molecule.

This also includes the enhancement of desirable properties in the drug and the suppression of undesirable properties. The specialized non-toxic protective group is referred to as “carrier”. A prodrug releases the reversibly and covalently bound drug moiety in the form of its corresponding drug. In other words, a prodrug is a conjugate comprising a drug moiety which is covalently and reversibly conjugated to a carrier moiety via a reversible linker moiety, which covalent and reversible conjugation of the carrier to the reversible linker moiety is either directly or through a spacer. Such conjugate releases the formerly conjugated drug moiety in the form of a free unmodified drug.

A “reversible linkage” is a linkage that is degradable, i.e., cleavable, in the absence of enzymes under physiological conditions (aqueous buffer at pH 7.4, 37° C.) with a half-life ranging from 1 hour to three months. A “stable linkage” is a linkage having a half-life under physiological conditions (aqueous buffer at pH 7.4, 37° C.) in the absence of enzymes of more than three months.

As used herein, the terms “traceless prodrug linker” or “traceless linker” means a reversible prodrug linker, i.e., a linker moiety reversibly and covalently connecting a drug moiety with a carrier, which upon cleavage releases the drug in its free form. As used herein, the term “free form” of a drug means the drug in its unmodified, pharmacologically active form.

As used herein, the term “reagent” means a chemical compound which comprises at least one functional group for reaction with the functional group of another chemical compound or drug. It is understood that a drug comprising a functional group, such as a primary or secondary amine or hydroxyl functional group, is also a reagent.

As used herein, the term “moiety” means a part of a molecule, which lacks one or more atom(s) compared to the corresponding reagent. If, for example, a reagent of the formula “H—X—H” reacts with another reagent and becomes part of the reaction product, the corresponding moiety of the reaction product has the structure “H—X—” or “—X—”, whereas each “-” indicates attachment to another moiety. Accordingly, a drug moiety is released from a prodrug as a drug.

If a chemical structure of a group of atoms is provided, which group of atoms is attached to at least one other moiety, said chemical structure may be attached to the at least one other moiety in either orientation, unless explicitly stated otherwise. For example, a moiety “—C(O)N(R¹)—” may be attached to two moieties either as “—C(O)N(R¹)—” or as “—N(R′)C(O)—”. Similarly, a moiety

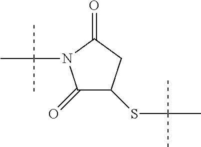

may be attached to two moieties either as

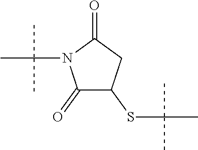

or as

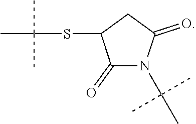

As used herein, the term “functional group” means a group of atoms which can react with other groups of atoms. Functional groups are for example selected from the group consisting of carboxylic acid, primary or secondary amine, maleimide, thiol, sulfonic acid, carbonate, carbamate, hydroxyl, aldehyde, ketone, hydrazine, isocyanate, isothiocyanate, phosphoric acid, phosphonic acid, haloacetyl, alkyl halide, acryloyl, aryl fluoride, hydroxylamine, disulfide, sulfonamides, sulfuric acid, vinyl sulfone, vinyl ketone, diazoalkane, oxirane and aziridine.

In case a compound, such as a conjugate of the present invention, comprises one or more acidic or basic groups, the invention also comprises its corresponding pharmaceutically or toxicologically acceptable salts, in particular their pharmaceutically utilizable salts. Thus, a compound comprising acidic groups may be used according to the invention, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine or amino acids. A compound comprising one or more basic groups, i.e., groups which can be protonated, may be present and may be used according to the invention in the form of their addition salts with inorganic or organic acids. Examples for suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. For the person skilled in the art further methods are known for converting the basic group into a cation like the alkylation of an amine group resulting in a positively-charge ammonium group and an appropriate counterion of the salt. If the compound simultaneously comprises acidic and basic groups, the invention also includes, in addition to the salt forms mentioned above, inner salts or betaines (zwitterions). The respective salts may be obtained by customary methods which are known to the person skilled in the art like, for example by contacting these compounds with an organic or inorganic acid or base in a solvent or dispersant, or by anion exchange or cation exchange with other salts. The present invention also includes all salts of the compounds which, owing to low physiological compatibility, are not directly suitable for use in pharmaceuticals but which can be used, for example, as intermediates for chemical reactions or for the preparation of pharmaceutically acceptable salts.

The term “pharmaceutically acceptable” means a substance that does not cause harm when administered to a patient and in certain embodiments means approved by a regulatory agency, such as the EMA (Europe) and/or the FDA (US) and/or any other national regulatory agency for use in animals, in particular for use in humans.

As used herein the terms “about” or “approx.” in combination with a numerical value is used to indicate a range ranging from and including the numerical value plus and minus no more than 10% of said numerical value. For example, the phrases “about 200” or “approx. 200” is used to mean a range ranging from and including 200+/−10%, i.e. ranging from and including 180 to 220. It is understood that a percentage given as “about 20%” or “approx. 20%” does not mean “20%+/−10%”, i.e. ranging from and including 10 to 30%, but “about 20%” or “approx. 20%” means ranging from and including 18 to 22%, i.e. plus and minus 10% of the numerical value which is 20.

As used herein, the term “polymer” means a molecule comprising repeating structural units, i.e., the monomers, connected by chemical bonds in a linear, circular, branched, crosslinked or dendrimeric way or a combination thereof, which may be of synthetic or biological origin or a combination of both. It is understood that a polymer may also comprise one or more other chemical groups and/or moieties, such as, for example, one or more functional groups. In certain embodiments a soluble polymer has a molecular weight of at least 0.5 kDa, e.g., a molecular weight of at least 1 kDa, a molecular weight of at least 2 kDa, a molecular weight of at least 3 kDa or a molecular weight of at least 5 kDa. If the polymer is soluble, it in certain embodiments has a molecular weight of at most 1000 kDa, such as at most 750 kDa, such as at most 500 kDa, such as at most 300 kDa, such as at most 200 kDa, or such as at most 100 kDa. It is understood that for water-insoluble polymers, such as hydrogels, no meaningful molecular weight ranges can be provided. It is understood that also a peptide or protein is a polymer in which the amino acids are the repeating structural units, even though the side chains of each amino acid may be different.

As used herein, the term “polymeric” means a reagent or a moiety comprising one or more polymers or polymer moieties. A polymeric reagent or moiety may optionally also comprise one or more other moiety/moieties, which are in certain embodiments selected from the group consisting of:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl; and
linkages selected from the group comprising

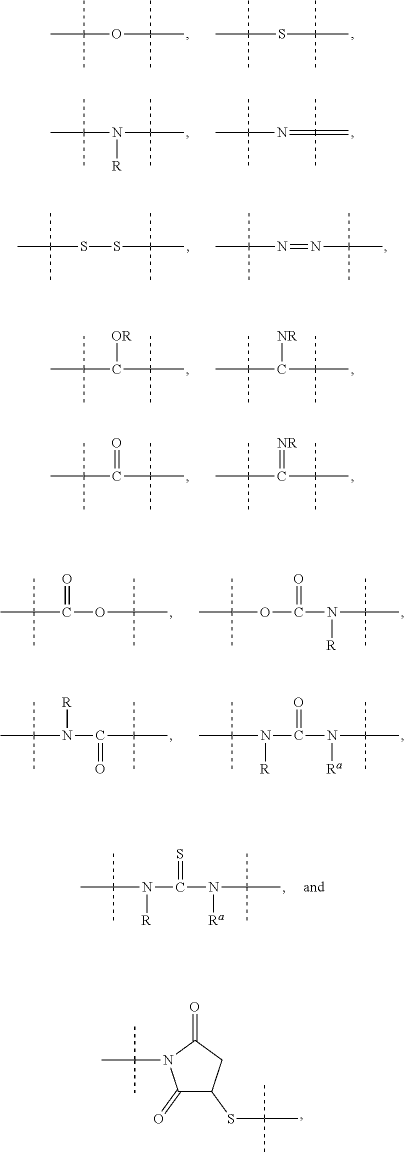

wherein
dashed lines indicate attachment to the remainder of the moiety or reagent, and —R and —R^{a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

The person skilled in the art understands that the polymerization products obtained from a polymerization reaction do not all have the same molecular weight, but rather exhibit a molecular weight distribution. Consequently, the molecular weight ranges, molecular weights, ranges of numbers of monomers in a polymer and numbers of monomers in a polymer as used herein, refer to the number average molecular weight and number average of monomers, i.e., to the arithmetic mean of the molecular weight of the polymer or polymeric moiety and the arithmetic mean of the number of monomers of the polymer or polymeric moiety.

Accordingly, in a polymeric moiety comprising “x” monomer units any integer given for “x” therefore corresponds to the arithmetic mean number of monomers. Any range of integers given for “x” provides the range of integers in which the arithmetic mean numbers of monomers lie. An integer for “x” given as “about x” means that the arithmetic mean numbers of monomers lie in a range of integers of x+/−10%, in certain embodiments x+/−8%, in certain embodiments x+/−5% and in certain embodiments x+/−2%.

As used herein, the term “number average molecular weight” means the ordinary arithmetic mean of the molecular weights of the individual polymers.

As used herein the term “water-soluble” with reference to the conjugate of the present invention means that at least 1 g of the conjugate may be dissolved in one liter of water at 20° C. to form a homogeneous solution. Accordingly, the term “water-insoluble” with reference to the compound means that less than 1 g of the conjugate may be dissolved in one liter of water at 20° C. to form a homogeneous solution.

As used herein, the term “PEG-based” in relation to a moiety or reagent means that said moiety or reagent comprises PEG. In certain embodiments a PEG-based moiety or reagent comprises at least 10% (w/w) PEG, such as at least 20% (w/w) PEG, such as at least 30% (w/w) PEG, such as at least 40% (w/w) PEG, such as at least 50% (w/w), such as at least 60 (w/w) PEG, such as at least 70% (w/w) PEG, such as at least 80% (w/w) PEG, such as at least 90% (w/w) PEG, such as at least 95%. The remaining weight percentage of the PEG-based moiety or reagent are other moieties that in certain embodiments are selected from the following moieties and linkages:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl; and
linkages selected from the group comprising

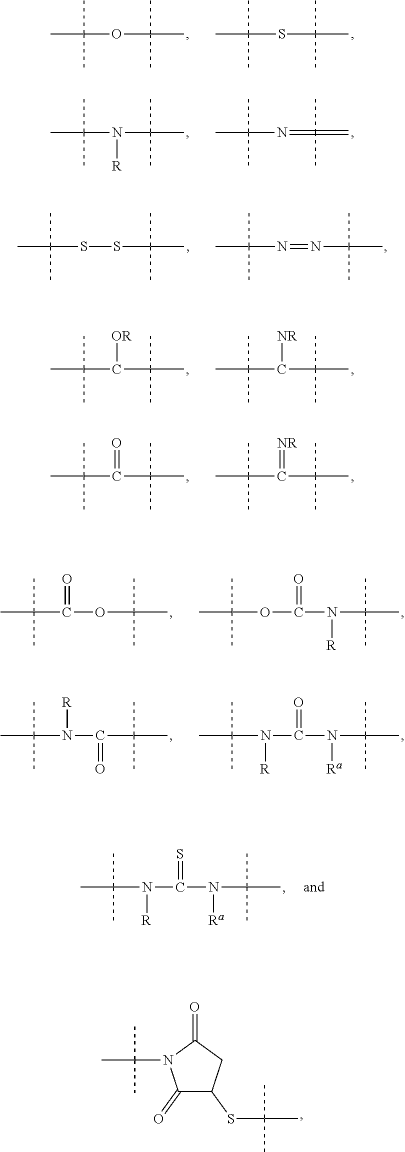

wherein
dashed lines indicate attachment to the remainder of the moiety or reagent, and —R and —R^{a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

As used herein, the term “monocyclic or bicyclic aryl” means an aromatic hydrocarbon ring system which may be monocyclic or bicyclic, wherein the monocyclic aryl ring consists of at least 5 ring carbon atoms and may comprise up to 10 ring carbon atoms and wherein the bicylic aryl ring consists of at least 8 ring carbon atoms and may comprise up to 12 ring carbon atoms. Each hydrogen atom of a monocyclic or bicyclic aryl may be replaced by a substituent as defined below.

As used herein, the term “monocyclic or bicyclic heteroaryl” means a monocyclic aromatic ring system that may comprise 2 to 6 ring carbon atoms and 1 to 3 ring heteroatoms or a bicyclic aromatic ring system that may comprise 3 to 9 ring carbon atoms and 1 to 5 ring heteroatoms, such as nitrogen, oxygen and sulfur. Examples for monocyclic or bicyclic heteroaryl groups include, but are not limited to, benzofuranyl, benzothiophenyl, furanyl, imidazolyl, indolyl, azaindolyl, azabenzimidazolyl, benzoxazolyl, benzthiazolyl, benzthiadiazolyl, benzotriazolyl, tetrazinyl, tetrazolyl, isothiazolyl, oxazolyl, isoxazolyl, pyrazinyl, pyrazolyl, pyridazinyl, pyridinyl, pyrimidinyl, pyrrolyl, quinolinyl, quinazolinyl, quinoxalinyl, triazolyl, thiazolyl and thiophenyl. Each hydrogen atom of a monocyclic or bicyclic heteroaryl may be replaced by a substituent as defined below.

As used herein, the term “cleavable triggering moiety (-Tr)” refers to a moiety that may undergo chemical or enzymatic cleavage under physiological conditions (aqueous buffer at pH 7.4, 37° C.), wherein upon cleavage of said cleavable triggering moiety a drug conjugate will release at least two drug moieties comprising an albumin-binding moiety.

The term “substituted” as used herein means that one or more —H atom(s) of a molecule or moiety are replaced by a different atom or a group of atoms, which are referred to as “substituent”.

In certain embodiments the one or more further optional substituents are independently of each other selected from the group consisting of halogen, —CN, —COOR^{x1}, —OR^{x1}, —C(O)R^{x1}, —C(O)N(R^{x1}R^{x1a}), —S(O)₂N(R^{x1}R^{x1a}), —S(O)N(R^{x1}R^{x1a}), —S(O)₂R^{x1}, —S(O)R^{x1}, —N(R^{x1})S(O)₂N(R^{x1a}R^{x1b}), —SR^{x1}, —N(R^{x1}R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a}R^{x1b}), —OC(O)N(R^{x1}R^{x1a}), -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R², which are the same or different and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;
—R^{x}, —R^{x1a}, R^{x1b} are independently of each other selected from the group consisting of —H, -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R², which are the same or different and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—; —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R², which are the same or different;
each —R^{x2} is independently selected from the group consisting of halogen, —CN, oxo (═O), —COOR^{x4}, —OR^{x4}, —C(O)R^{x4}, —C(O)N(R^{x4}R^{x4a}), —S(O)₂N(R^{x4}R^{x4a}), —S(O)N(R^{x4}R^{x4a}), —S(O)₂R^{x4}, —S(O)R^{x4}, —N(R^{x4})S(O)₂N(R^{x4a}R^{x4b}), —SR^{x4}, —N(R^{x4}R^{x4a}), —NO₂, —OC(O)R^{x4}, —N(R^{x4})C(O)R^{x4a}, —N(R^{x4})S(O)₂R^{x4a}, —N(R^{x4})S(O)R^{x4a}, —N(R^{x4})C(O)OR^{x4a}, —N(R^{x4})C(O)N(R^{x4a}R^{x4b}), —OC(O)N(R^{x4}R^{x4a}), and C₁₋₆ alkyl; wherein C₁₋₆ alkyl is optionally substituted with one or more halogen, which are the same or different;
each —R^{x3}, —R^{x3a}, —R^{x4}, —R^{x4a}, —R^{x4b} is independently selected from the group consisting of —H and C₁₋₆ alkyl; wherein C₁₋₆ alkyl is optionally substituted with one or more halogen, which are the same or different.

In certain embodiments the one or more further optional substituents are independently of each other selected from the group consisting of halogen, —CN, —COOR^{x1}, —OR^{x1}, —C(O)R^{x1}, —C(O)N(R^{x1}R^{x1a}), —S(O)₂N(R^{x1}R^{x1a}), —S(O)N(R^{x1}R^{x1a}), —S(O)₂R^{x1}, —S(O)R^{x1}, —N(R^{x1})S(O)₂N(R^{x1a}R^{x1b}), —SR^{x1}, —N(R^{x1}R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a}R^{x1b}), —OC(O)N(R^{x1}R^{x1a}), -T⁰, C₁₋₁₀ alkyl, C₂₋₁₀ alkenyl, and C₂₋₁₀ alkynyl; wherein -T⁰, C₁₋₁₀ alkyl, C₂₋₁₀ alkenyl, and C₂₋₁₀ alkynyl are optionally substituted with one or more —R², which are the same or different and wherein C₁₋₁₀ alkyl, C₂₋₁₀ alkenyl, and C₂₋₁₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;
each —R^{x1}, —R^{x1a}, —R^{x1b}, —R^{x3}, R^{x3a} is independently selected from the group consisting of —H, halogen, C₁₋₆ alkyl, C₂₋₆ alkenyl, and C₂₋₆ alkynyl;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R², which are the same or different;
each —R² is independently selected from the group consisting of halogen, —CN, oxo (═O), —COOR^{x4}, —OR^{x4}, —C(O)R^{x4}, —C(O)N(R^{x4}R^{x4a}), —S(O)₂N(R^{x4}R^{x4a}), —S(O)N(R^{x4}R^{x4a}), —S(O)₂R^{x4}, —S(O)R^{x4}, —N(R^{x4})S(O)₂N(R^{x4a}R^{x4b}), —SR^{x4}, —N(R^{x4}R^{x4a}), —NO₂, —OC(O)R^{x4}, —N(R^{x4})C(O)R^{x4a}, —N(R^{x4})S(O)₂R^{x4a}, —N(R^{x4})S(O)R^{x4a}, —N(R^{x4})C(O)OR^{x4a}, —N(R^{x4})C(O)N(R^{x4a}R^{x4b}), —OC(O)N(R^{x4}R^{x4a}), and C₁₋₆ alkyl; wherein C₁₋₆ alkyl is optionally substituted with one or more halogen, which are the same or different;
each —R^{x4}, —R^{x4a}, —R^{x4b} is independently selected from the group consisting of —H, halogen, C₁₋₆ alkyl, C₂₋₆ alkenyl, and C₂₋₆ alkynyl;

In certain embodiments the one or more further optional substituents are independently of each other selected from the group consisting of halogen, —CN, —COOR^{x1}, —OR^{x1}, —C(O)R^{x1}, —C(O)N(R^{x1}R^{x1a}), —S(O)₂N(R^{x1}R^{x1a}), —S(O)N(R^{x1}R^{x1a}), —S(O)₂R^{x1}, —S(O)R^{x1}, —N(R^{x1})S(O)₂N(R^{x1a}R^{x1b}), —SR^{x1}, —N(R^{x1}R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a}R^{x1b}), —OC(O)N(R^{x1}R^{x1a}), -T⁰, C₁₋₆ alkyl, C₂₋₆ alkenyl, and C₂₋₆ alkynyl; wherein -T⁰, C₁₋₆ alkyl, C₂₋₆ alkenyl, and C₂₋₆ alkynyl are optionally substituted with one or more —R², which are the same or different and wherein C₁₋₆ alkyl, C₂₋₆ alkenyl, and C₂₋₆ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;
each —R^{x1}, —R^{x1a}, —R^{x1b}, —R³, —R^{x3}, a is independently selected from the group consisting of —H, halogen, C₁₋₆ alkyl, C₂₋₆ alkenyl, and C₂₋₆ alkynyl;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2}, which are the same or different.

In certain embodiments a maximum of 6 —H atoms of an optionally substituted molecule are independently replaced by a substituent, e.g. 5 —H atoms are independently replaced by a substituent, 4 —H atoms are independently replaced by a substituent, 3 —H atoms are independently replaced by a substituent, 2 —H atoms are independently replaced by a substituent, or 1 —H atom is replaced by a substituent.

The term “interrupted” means that a moiety is inserted between two carbon atoms or—if the insertion is at one of the moiety's ends—between a carbon or heteroatom and a hydrogen atom, in certain embodiments between a carbon and a hydrogen atom.

As used herein, the term “C₁₋₄ alkyl” alone or in combination means a straight-chain or branched alkyl moiety having 1 to 4 carbon atoms. If present at the end of a molecule, examples of straight-chain or branched C₁₋₄ alkyl are methyl, ethyl, n-propyl, isopropyl, n-butyl, isobutyl, sec-butyl and tert-butyl. When two moieties of a molecule are linked by the C₁₋₄ alkyl, then examples for such C₁₋₄ alkyl groups are —CH₂—, —CH₂—CH₂—, —CH(CH₃)—, —CH₂—CH₂—CH₂—, —CH(C₂H₅)—, —C(CH₃)₂—. Each hydrogen of a C₁₋₄ alkyl carbon may optionally be replaced by a substituent as defined above. Optionally, a C₁₋₄ alkyl may be interrupted by one or more moieties as defined below.

As used herein, the term “C₁₋₆ alkyl” alone or in combination means a straight-chain or branched alkyl moiety having 1 to 6 carbon atoms. If present at the end of a molecule, examples of straight-chain and branched C₁₋₆ alkyl groups are methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. When two moieties of a molecule are linked by the C₁₋₆ alkyl group, then examples for such C₁₋₆ alkyl groups are —CH₂—, —CH₂—CH₂—, —CH(CH₃)—, —CH₂—CH₂—CH₂—, —CH(C₂H₅)— and —C(CH₃)₂—. Each hydrogen atom of a C₁₋₆ carbon may optionally be replaced by a substituent as defined above. Optionally, a C₁₋₆ alkyl may be interrupted by one or more moieties as defined below.

Accordingly, “C₁₋₁₀ alkyl”, “C₁₋₂₀ alkyl” or “C₁₋₅₀ alkyl” means an alkyl chain having 1 to 10, 1 to 20 or 1 to 50 carbon atoms, respectively, wherein each hydrogen atom of the C₁₋₁₀, C₁₋₂₀ or C₁₋₅₀ carbon may optionally be replaced by a substituent as defined above. Optionally, a C₁₋₁₀ or C₁₋₅₀ alkyl may be interrupted by one or more moieties as defined below.

As used herein, the term “C₂₋₆ alkenyl” alone or in combination means a straight-chain or branched hydrocarbon moiety comprising at least one carbon-carbon double bond having 2 to 6 carbon atoms. If present at the end of a molecule, examples are —CH═CH₂, —CH═CH—CH₃, —CH₂—CH═CH₂, —CH═CHCH₂—CH₃ and —CH═CH—CH═CH₂. When two moieties of a molecule are linked by the C₂₋₆ alkenyl group, then an example for such C₂₋₆ alkenyl is —CH═CH—. Each hydrogen atom of a C₂₋₆ alkenyl moiety may optionally be replaced by a substituent as defined above. Optionally, a C₂₋₆ alkenyl may be interrupted by one or more moieties as defined below.

Accordingly, the term “C₂₋₁₀ alkenyl”, “C₂₋₂₀ alkenyl” or “C₂₋₅₀ alkenyl” alone or in combination means a straight-chain or branched hydrocarbon moiety comprising at least one carbon-carbon double bond having 2 to 10, 2 to 20 or 2 to 50 carbon atoms. Each hydrogen atom of a C₂₋₁₀ alkenyl, C₂₋₂₀ alkenyl or C₂₋₅₀ alkenyl group may optionally be replaced by a substituent as defined above. Optionally, a C₂₋₁₀ alkenyl, C₂₋₂₀ alkenyl or C₂₋₅₀ alkenyl may be interrupted by one or more moieties as defined below.

As used herein, the term “C₂₋₆ alkynyl” alone or in combination means straight-chain or branched hydrocarbon moiety comprising at least one carbon-carbon triple bond having 2 to 6 carbon atoms. If present at the end of a molecule, examples are —C≡CH, —CH₂—C≡CH, CH₂—CH₂—C≡CH and CH₂—C≡C—CH₃. When two moieties of a molecule are linked by the alkynyl group, then an example is —C≡C—. Each hydrogen atom of a C₂₋₆ alkynyl group may optionally be replaced by a substituent as defined above. Optionally, one or more double bond(s) may occur. Optionally, a C₂₋₆ alkynyl may be interrupted by one or more moieties as defined below.

Accordingly, as used herein, the term “C₂₋₁₀ alkynyl”, “C₂₋₂₀ alkynyl” and “C₂₋₅₀ alkynyl” alone or in combination means a straight-chain or branched hydrocarbon moiety comprising at least one carbon-carbon triple bond having 2 to 10, 2 to 20 or 2 to 50 carbon atoms, respectively.

Each hydrogen atom of a C₂₋₁₀ alkynyl, C₂₋₂₀ alkynyl or C₂₋₅₀ alkynyl group may optionally be replaced by a substituent as defined above. Optionally, one or more double bond(s) may occur.

Optionally, a C₂₋₁₀ alkynyl, C₂₋₂₀ alkynyl or C₂₋₅₀ alkynyl may be interrupted by one or more moieties as defined below.

As mentioned above, a C₁₋₄ alkyl, C₁₋₆ alkyl, C₁₋₁₀ alkyl, C₁₋₂₀ alkyl, C₁₋₅₀ alkyl, C₂₋₆ alkenyl, C₂₋₁₀ alkenyl, C₂₋₂₀ alkenyl, C₂₋₅₀ alkenyl, C₂₋₆ alkynyl, C₂₋₁₀ alkynyl, C₂₋₂₀ alkenyl or C₂₋₅₀ alkynyl may optionally be interrupted by one or more moieties which in certain embodiments are selected from the group consisting of

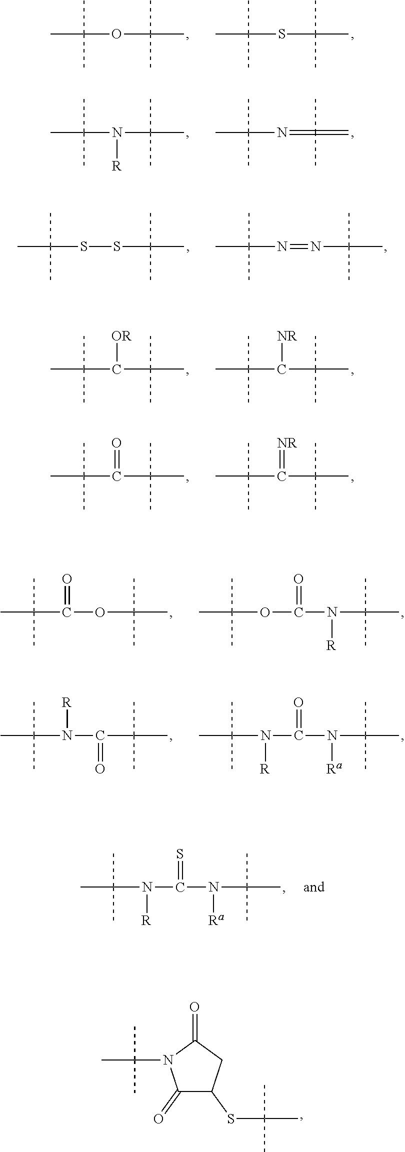

wherein
dashed lines indicate attachment to the remainder of the moiety or reagent; and —R and —R^{a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

As used herein, the term “C₃₋₁₀ cycloalkyl” means a cyclic alkyl chain having 3 to 10 carbon atoms, which may be saturated or unsaturated, e.g. cyclopropyl, cyclobutyl, cyclopentyl, cyclohexyl, cyclohexenyl, cycloheptyl, cyclooctyl, cyclononyl or cyclodecyl. Each hydrogen atom of a C₃₋₁₀ cycloalkyl carbon may be replaced by a substituent as defined above. The term “C₃₋₁₀ cycloalkyl” also includes bridged bicycles like norbornane or norbornene.

The term “8- to 30-membered carbopolycyclyl” or “8- to 30-membered carbopolycycle” means a cyclic moiety of two or more rings with 8 to 30 ring atoms, where two neighboring rings share at least one ring atom and that may contain up to the maximum number of double bonds (aromatic or non-aromatic ring which is fully, partially or un-saturated). In certain embodiments an 8- to 30-membered carbopolycyclyl means a cyclic moiety of two, three, four or five rings, in certain embodiments of two, three or four rings.

As used herein, the term “3- to 10-membered heterocyclyl” or “3- to 10-membered heterocycle” means a ring with 3, 4, 5, 6, 7, 8, 9 or 10 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for 3- to 10-membered heterocycles include but are not limited to aziridine, oxirane, thiirane, azirine, oxirene, thiirene, azetidine, oxetane, thietane, furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepane, azepine and homopiperazine. Each hydrogen atom of a 3- to 10-membered heterocyclyl or 3-to 10-membered heterocyclic group may be replaced by a substituent as defined below.

As used herein, the term “8- to 11-membered heterobicyclyl” or “8- to 11-membered heterobicycle” means a heterocyclic moiety of two rings with 8 to 11 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for an 8- to 11-membered heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquinoline, decahydroquinoline, isoquinoline, decahydroisoquinoline, tetrahydroisoquinoline, dihydroisoquinoline, benzazepine, purine and pteridine. The term 8- to 11-membered heterobicycle also includes spiro structures of two rings like 1,4-dioxa-8-azaspiro[4.5]decane or bridged heterocycles like 8-aza-bicyclo[3.2.1]octane. Each hydrogen atom of an 8- to 11-membered heterobicyclyl or 8- to 11-membered heterobicycle carbon may be replaced by a substituent as defined below.

Similarly, the term “8- to 30-membered heteropolycyclyl” or “8- to 30-membered heteropolycycle” means a heterocyclic moiety of more than two rings with 8 to 30 ring atoms, in certain embodiments of three, four or five rings, where two neighboring rings share at least one ring atom and that may contain up to the maximum number of double bonds (aromatic or non-aromatic ring which is fully, partially or unsaturated), wherein at least one ring atom up to 10 ring atoms are replaced by a heteroatom selected from the group of sulfur (including —S(O)— and —S(O)₂—), oxygen and nitrogen (including ═N(O)—) and wherein the ring is linked to the rest of a molecule via a carbon or nitrogen atom.

It is understood that the phrase “the pair R^{x}/R^{y} is joined together with the atom to which they are attached to form a C₃₋₁₀ cycloalkyl or a 3- to 10-membered heterocyclyl” in relation with a moiety of the structure

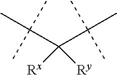

means that R^{x} and R^{y} form the following structure:

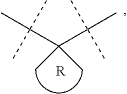

wherein R is C₃₋₁₀ cycloalkyl or 3- to 10-membered heterocyclyl.

It is also understood that the phrase “the pair R^{x}/R^{y} is joint together with the atoms to which they are attached to form a ring A” in relation with a moiety of the structure

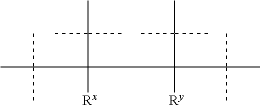

means that R^{x} and R^{y} form the following structure:

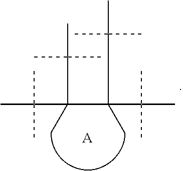

As used herein, “halogen” means fluoro, chloro, bromo or iodo. In certain embodiments halogen is fluoro or chloro.

In general, the term “comprise” or “comprising” also encompasses “consist of” or “consisting of”.

In certain embodiments the half-life of the drug released from the compound is it at least 1.5-fold higher than the corresponding free drug's half-life. In certain embodiments the half-life of the drug released from the compound is it at least 2.5-fold higher than the corresponding free drug's half-life. In certain embodiments the half-life of the drug released from the compound is it at least 5-fold higher than the corresponding free drug's half-life. In certain embodiments the half-life of the drug released from the compound is it at least 7.5-fold higher than the corresponding free drug's half-life. In certain embodiments the half-life of the drug released from the compound is it at least 10-fold higher than the corresponding free drug's half-life.

In certain embodiments the release half-life of the compound is at least the circulation half-life of the corresponding free drug. In certain embodiments the release half-life of the compound is at least 2-fold higher than the circulation half-life of the corresponding free drug. In certain embodiments the release half-life of the compound is at least 3-fold higher than the circulation half-life of the corresponding free drug.

In certain embodiments the compounds of the present invention are compounds comprising at least one polymeric moiety, to which the at least two covalently and reversibly conjugated drug moieties, each of the at least two drug moieties comprises an albumin-binding moiety, are conjugated. These conjugates will be described in the following paragraphs.

In certain embodiments the compound of the present invention is a compound comprising at least one polymeric moiety, to which at least two moieties of formula (I) are conjugated and/or to which at least one moiety of formula (I′) is conjugated, wherein formula (I) and (I′) are

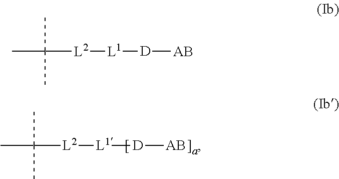

wherein
each -L²- is independently a spacer moiety or is absent;
each -L¹- is independently a linker moiety that is covalently and reversibly conjugated to -D-;
each -L^{1′}- is independently a linker moiety that is covalently and reversibly conjugated to -D-;
each -D- is independently a drug moiety;
each -AB is independently an albumin-binding moiety; and
each a is independently an integer selected from the group consisting of 1, 2, 3, 4, 5, 6, 7, 8,9, 10, 11, 12, 13, 14, 15 and 16.

Such compounds release a compound H-D-AB upon cleavage of the linkage between -L¹- and -D-, wherein H— is hydrogen. If a compound of the present invention for example comprises semaglutide in bound form, such as conjugated to -L¹- or -L^{1′}-, the semaglutide moiety -D-AB may for simplification be referred to herein as semaglutide, even though it strictly speaking would be a “semaglutide moiety”.

In certain embodiments such compound comprises at least one polymeric moiety, to which at least two moieties of formula (I) are conjugated, wherein formula (I) is

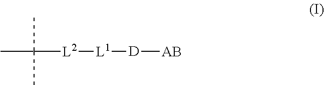

wherein
each -L²- is independently a spacer moiety or is absent;
each -L¹- is independently a linker moiety that is covalently and reversibly conjugated to -D-;
each -D- is independently a drug moiety;
each -AB is independently an albumin-binding moiety.

In certain embodiments such compound comprises at least one polymeric moiety, to which at least one moiety of formula (I′) is conjugated, wherein formula (I′) is

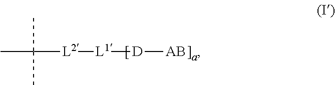

wherein
each -L²- is independently a spacer moiety or is absent;
each -L^{1′}- is independently a linker moiety that is covalently and reversibly conjugated to -D-;
each -D- is independently a drug moiety;
each -AB is independently an albumin-binding moiety; and
each a is independently an integer selected from the group consisting of 1, 2, 3, 4, 5, 6, 7, 8,9, 10, 11, 12, 13, 14, 15 and 16.

In certain embodiments the compound comprises two moieties of formula (I). In certain embodiments the compound comprises three moieties of formula (I). In certain embodiments the compound comprises four moieties of formula (I). In certain embodiments the compound comprises five moieties of formula (I). In certain embodiments the compound comprises six moieties of formula (I). In certain embodiments the compound comprises seven moieties of formula (I). In certain embodiments the compound comprises eight moieties of formula (I).

In certain embodiments the at least one polymeric moiety of the compounds of the present invention is a linear polymeric moiety —P—.

In certain embodiments a moiety of formula (I) or (I′) is conjugated to each of the two ends of such linear moiety —P—.

In certain embodiments the compound is of formula (I-a) or (I-a′)

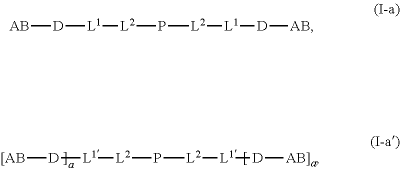

wherein
each -L²- is independently a spacer moiety or is absent;
each -L¹- is independently a linker moiety that is covalently and reversibly conjugated to -D-;
each -L^{1′}- is independently a linker moiety that is covalently and reversibly conjugated to -D-;
each -D- is independently a drug moiety;
each -AB is independently an albumin-binding moiety;
—P— is a polymeric moiety; and
each a is independently an integer selected from the group consisting of 1, 2, 3, 4, 5, 6, 7, 8,9, 10, 11, 12, 13, 14, 15 and 16.

In certain embodiments the compound is of formula (I-a). In certain embodiments the compound is of formula (I-a′).

It is understood that a moiety -L^{1′}- is conjugated to more than one moiety -D and that all linkages between -L^{1′}- and a moiety -D- are covalent and reversible.

In certain embodiments both a of formula (I-a′) are the same integer. In certain embodiments both a of formula (I-a′) are 2. In certain embodiments both a of formula (I-a′) are 3. In certain embodiments both a of formula (I-a′) are 4. In certain embodiments both a of formula (I-a′) are 5. In certain embodiments both a of formula (I-a′) are 6. In certain embodiments both a of formula (I-a′) are 7. In certain embodiments both a of formula (I-a′) are 8. In certain embodiments both a of formula (I-a′) are 9. In certain embodiments both a of formula (I-a′) are 10. In certain embodiments both a of formula (I-a′) are 11. In certain embodiments both a of formula (I-a′) are 12. In certain embodiments both a of formula (I-a′) are 13. In certain embodiments both a of formula (I-a′) are 14. In certain embodiments both a of formula (I-a′) are 15. In certain embodiments both a of formula (I-a′) are 16.

Optionally, further moieties of formula (I) or (I′) are conjugated to —P— of formula (I-a) or (I-a′).

In certain embodiments no further moieties of formula (I) or (I′) are conjugated to —P— of formula (I-a) or (I-a′).

A moiety —P— of formula (I-a) or (I-a′) comprises one or more polymer, such as a polymer selected from the group consisting of poly(2-methacryloyl-oxyethyl phosphoyl cholines), poly(acrylic acids), poly(acrylates), poly(acrylamides), poly(alkoxy) polymers, poly(amides), poly(amidoamines), poly(amino acids), poly(anhydrides), poly(aspartamides), poly(hydroxybutyrate), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethylene phosphates), poly(ethyloxazolines), poly(glycolic acids), poly(hydroxyethyl acrylates), poly(hydroxyethyl-oxazolines), poly(2-hydroxyethyl methacrylates), poly(N-(2-hydroxypropyl)methacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), celluloses, carboxymethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof.

A moiety —P— of formula (I-a) or (I-a′) comprises one or more polymer, such as a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof.

In certain embodiments —P— of formula (Ia) or (I-a′) is a PEG-based or hyaluronic acid-based moiety. In certain embodiments —P— of formula (Ia) or (I-a′) is a PEG-based moiety. In certain embodiments —P— of formula (Ia) or (I-a′) is a hyaluronic acid-based moiety.

In certain embodiments —P— of formula (Ia) or (I-a′) is of formula (I-ai)

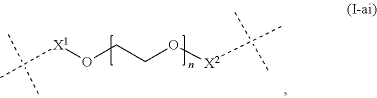

wherein
dashed lines indicate attachment to -L²-;
n is an integer such that the molecular weight of the PEG moiety ranges from about 1 about 20 kDa; —X¹— and —X²— are independently of each other selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR³)(R^{y3a})—, —N(R^{y3})C(O)N(R^{y3a})—, and —OC(O)N(R^{y3})—; —R^{y1} and —R^{y1a} are independently of each other selected from the group consisting of —H, -T, C₁₋₅₀ alkyl, C₂₋₅₀ alkenyl, and C₂₋₅₀ alkynyl; wherein -T, C₁₋₅₀ alkyl, C₂₋₅₀ alkenyl, and C₂₋₅₀ alkynyl are optionally substituted with one or more —R^{y2}, which are the same or different, and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR⁴)(R^{y4a})—, —N(R^{y4})C(O)N(R^{y4a})—, and —OC(O)N(R^{y4})—;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;
each —R^{y2} is independently selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5}) S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and
each —R^{y3}, —R^{y3a}, —R^{y4}, —R^{y4a}, —R^{y5}, —R^{y5a} and —R^{y5b} is independently selected from the group consisting of —H, and C₁₋₆ alkyl, wherein C₁₋₆ alkyl is optionally substituted with one or more halogen, which are the same or different.

In certain embodiments n of formula (I-ai) ranges from 2 to 22.

In certain embodiments —X¹— and —X²— of formula (I-ai) are independently of each other selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₂₀ alkyl, C₂₋₂₀ alkenyl, and C₂₋₂₀ alkynyl are optionally substituted with one or more —R^{y2}, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR³)(R^{y3a})—, —N(R^{y3})C(O)N(R^{y3a})—, and —OC(O)N(R^{y3})—;
—R^{y1} and —R^{y1a} are independently of each other selected from the group consisting of —H, -T, C₁₋₁₀ alkyl, C₂₋₁₀ alkenyl, and C₂₋₁₀ alkynyl; wherein -T, C₁₋₁₀ alkyl, C₂₋₁₀ alkenyl, and C₂₋₁₀ alkynyl are optionally substituted with one or more —R^{y2}, which are the same or different, and wherein C₁₋₁₀ alkyl, C₂₋₁₀ alkenyl, and C₂₋₁₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR⁴)(R^{y4a})—, —N(R^{y4})C(O)N(R^{y4a})—, and —OC(O)N(R^{y4})—;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;
—R^{y2} is selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and
each —R^{y3}, —R^{y3a}, —R^{y4}, —R^{y4a}, —R^{y5}, —R^{y1a} and —R^{y1b} is independently of each other selected from the group consisting of —H, and C₁₋₆ alkyl; wherein C₁₋₆ alkyl is optionally substituted with one or more halogen, which are the same or different.

In certain embodiments —X¹— and —X²— of formula (I-ai) are independently of each other selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR³)(R^{y3a})—, —N(R^{y3})C(O)N(R^{y3a})—, and —OC(O)N(R^{y3})—;
—R^{y1} and —R^{y1a} are independently selected from the group consisting of —H, -T, C₁₋₁₀ alkyl, C₂₋₁₀ alkenyl, and C₂₋₁₀ alkynyl;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl;
each —R^{y2} is independently selected from the group consisting of halogen, and C₁₋₆ alkyl; and
each —R^{y3}, —R^{y3a}, —R^{y4}, —R^{y4a}, —R^{y5}, —R^{y5a} and —R^{y5b} is independently of each other selected from the group consisting of —H, and C₁₋₆ alkyl; wherein C₁₋₆ alkyl is optionally substituted with one or more halogen, which are the same or different.

In certain embodiments —P— of formula (I-a) or (I-a′) is of formula (I-ai-i)

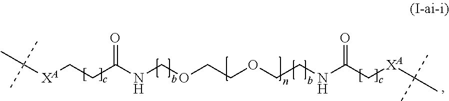

wherein
dashed lines indicate attachment to -L²-;
each —X^{A}— is independently selected from the group consisting of

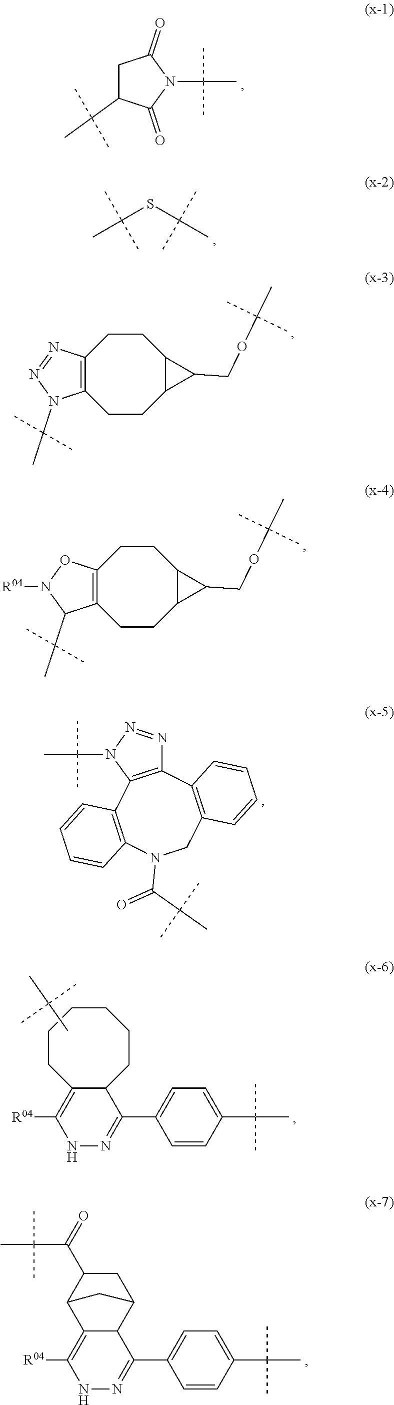

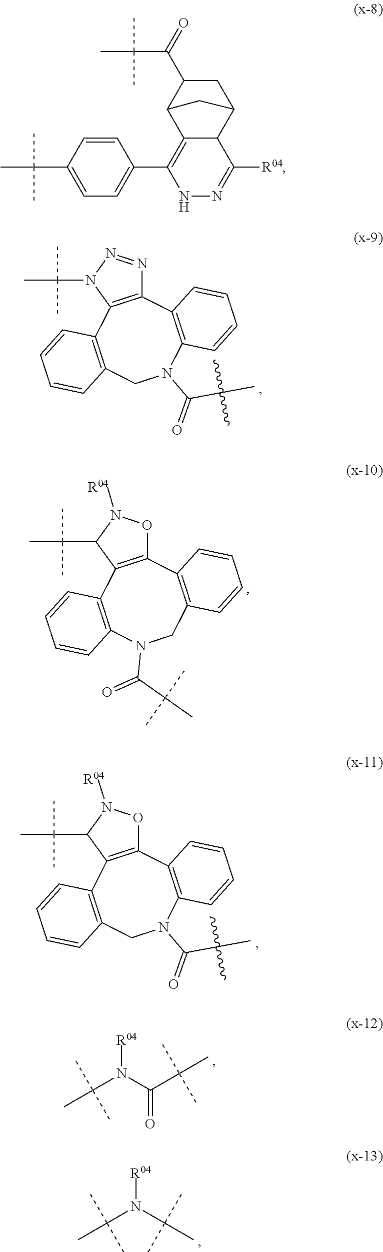

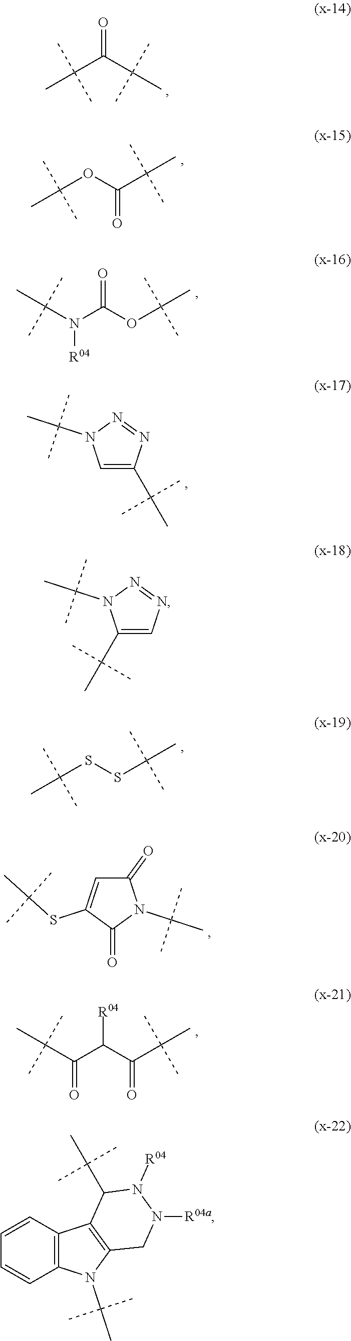

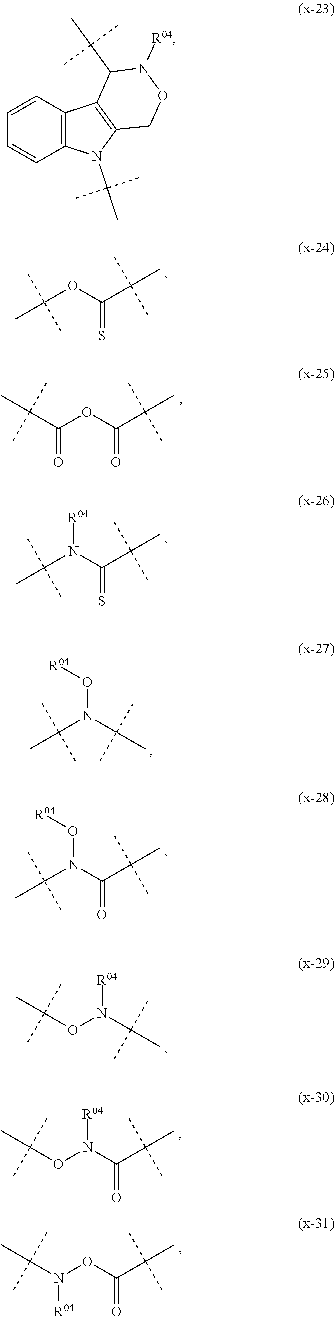

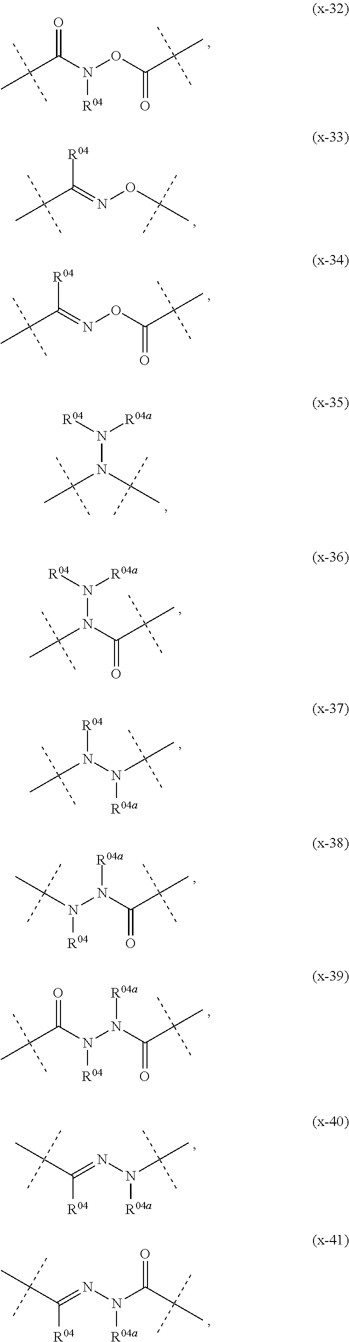

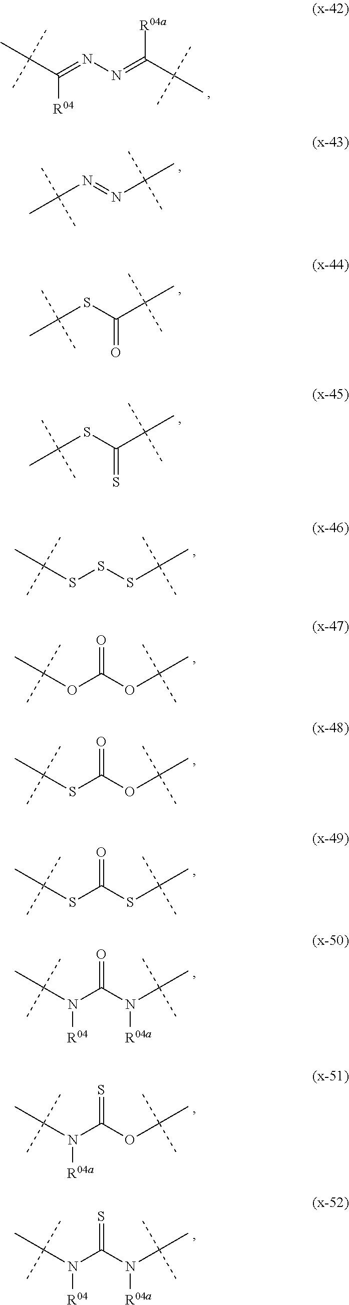

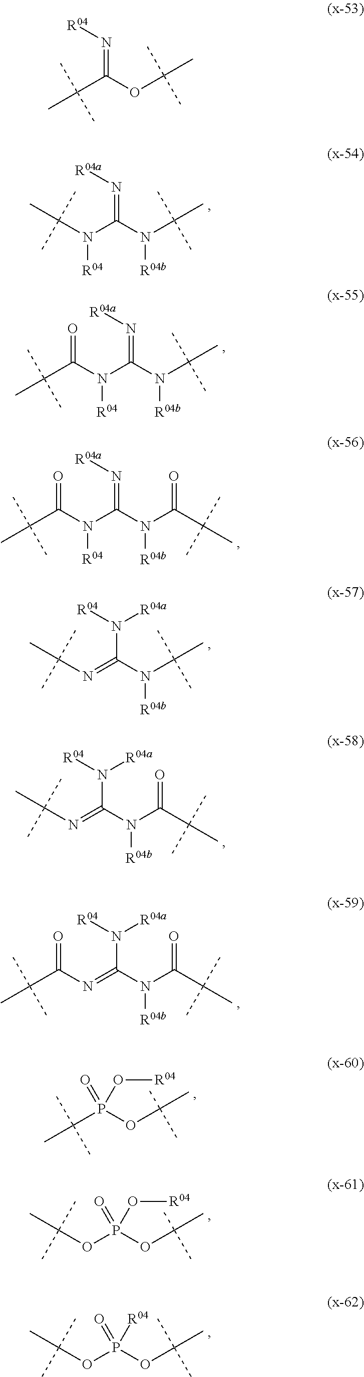

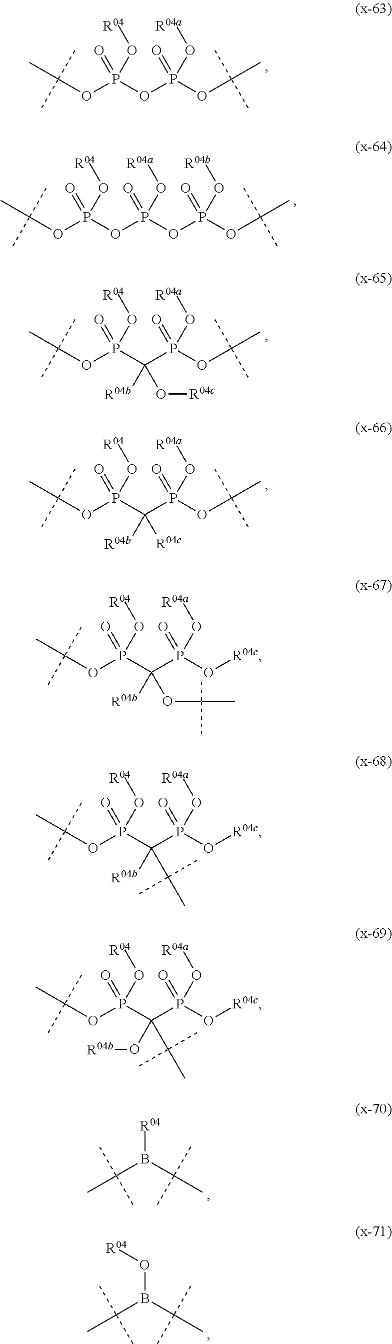

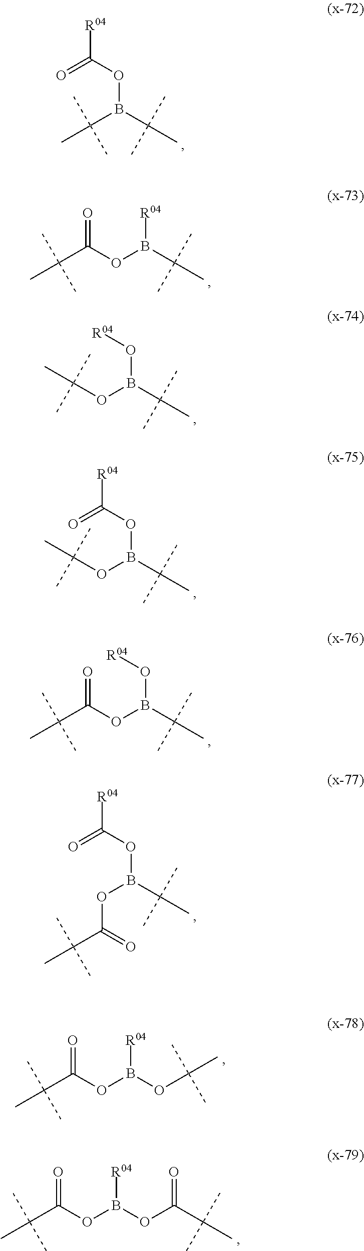

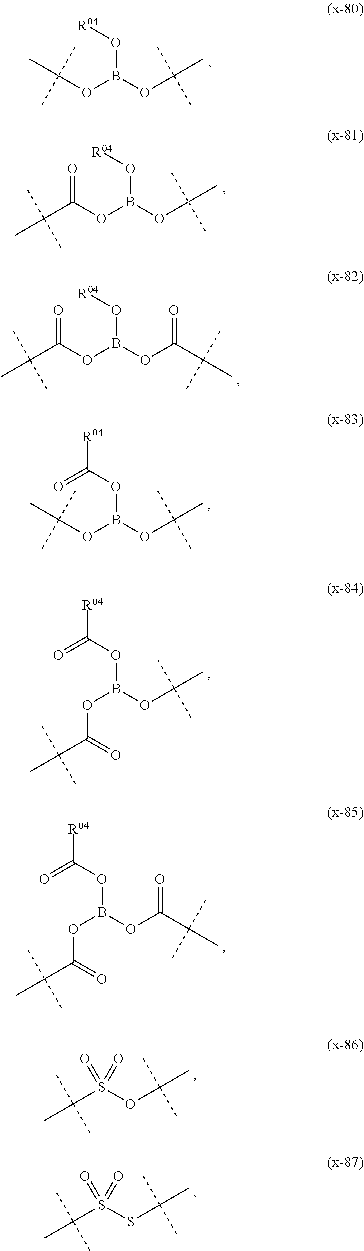

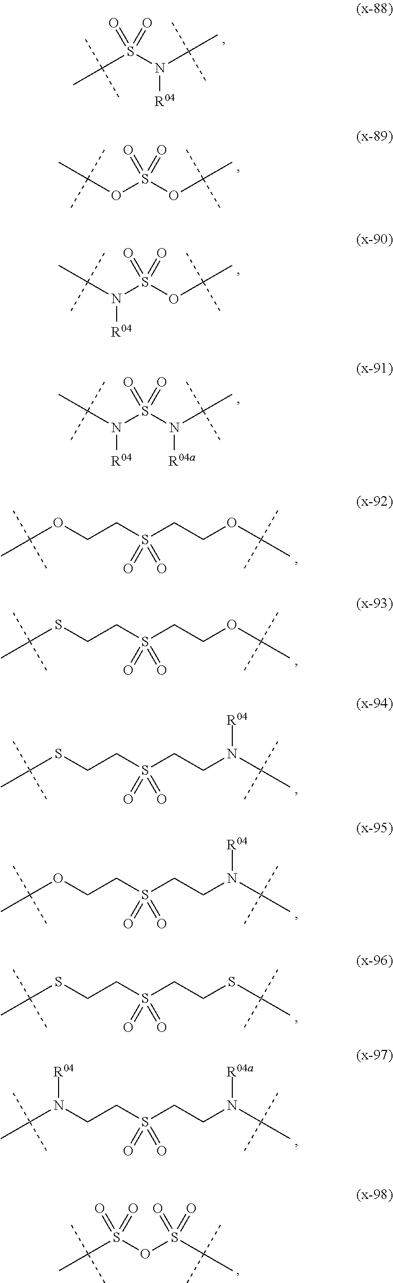

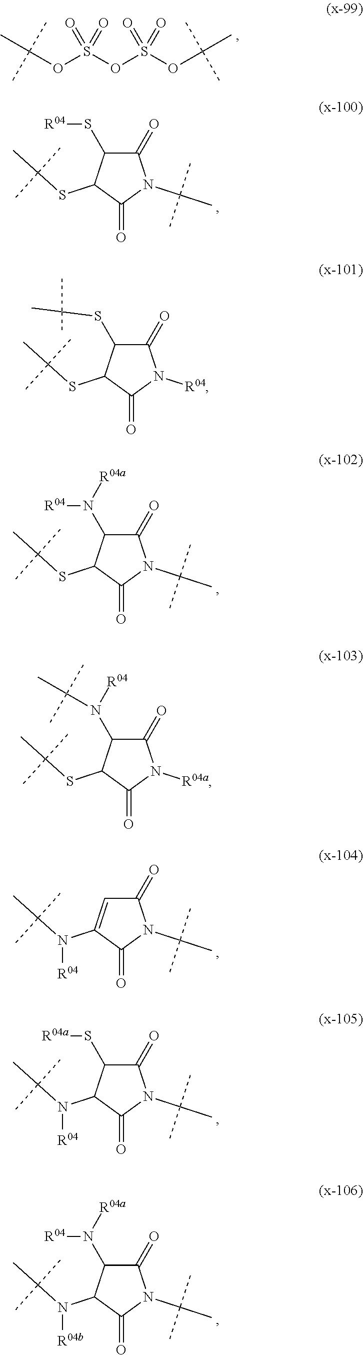

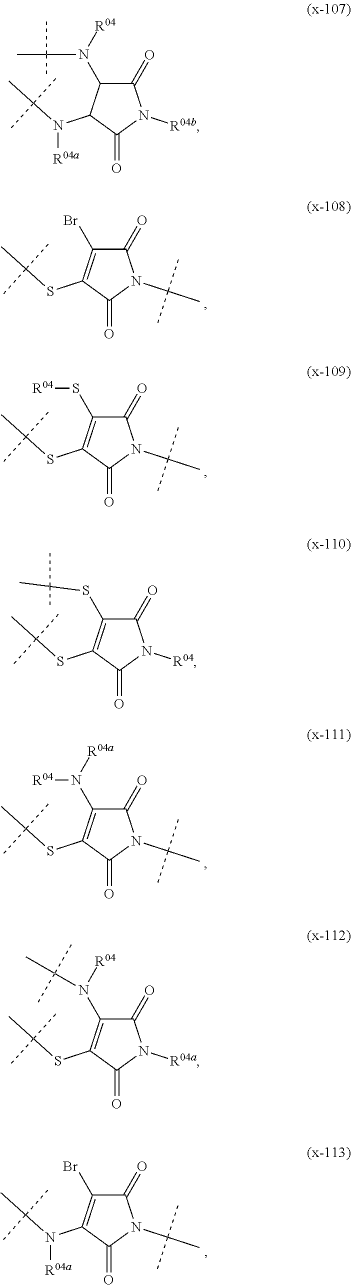

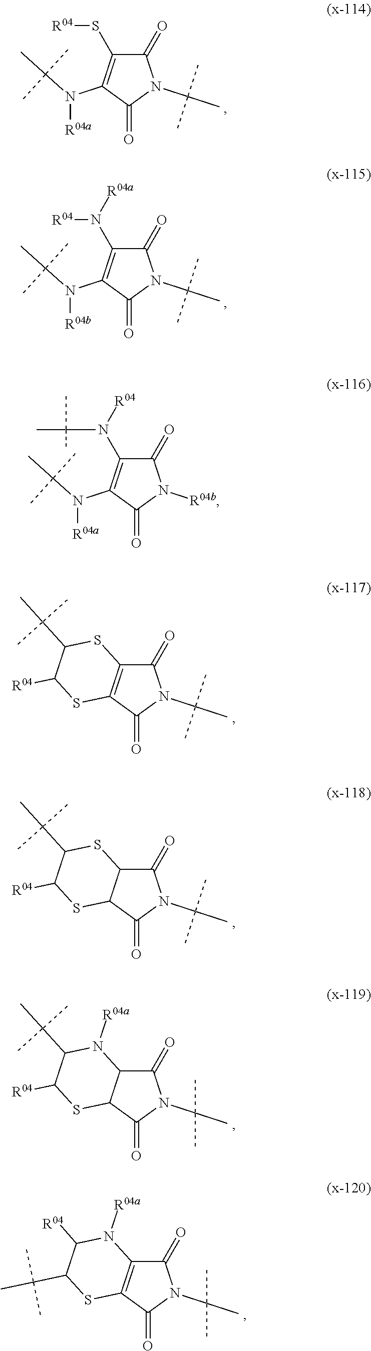

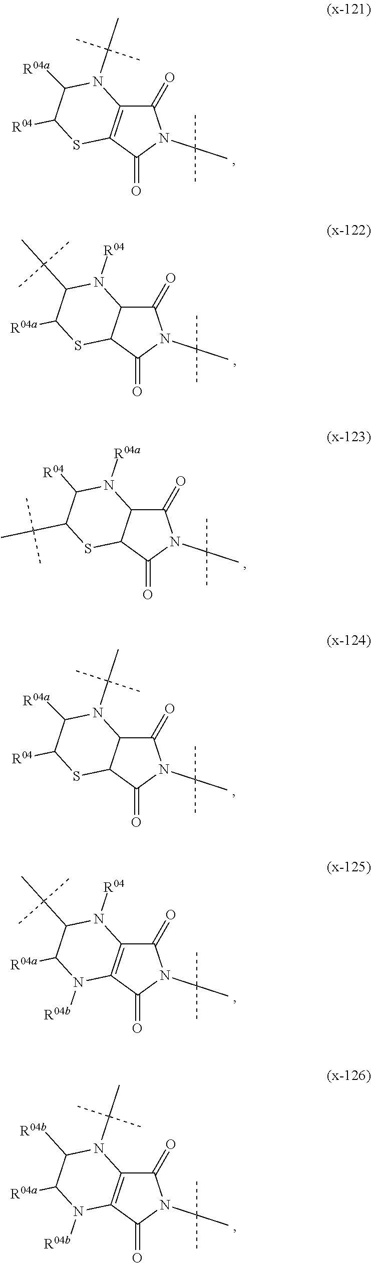

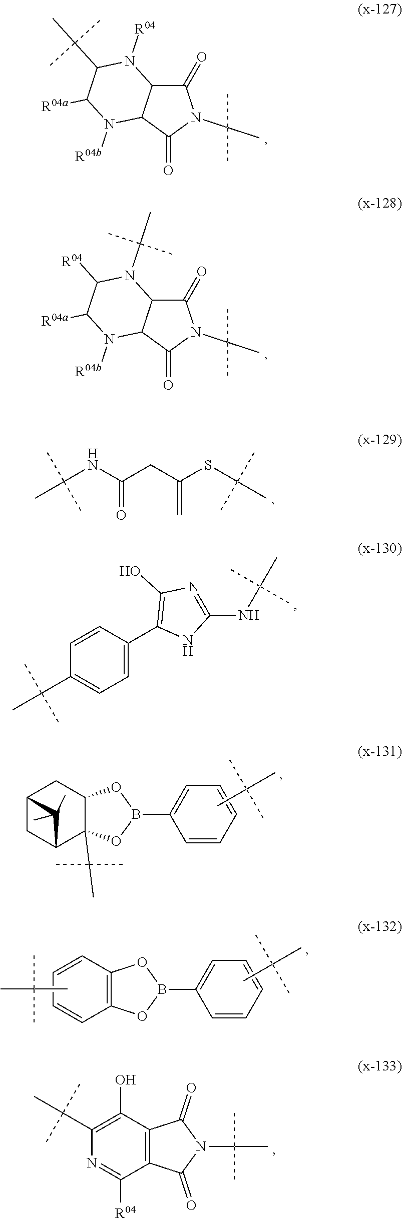

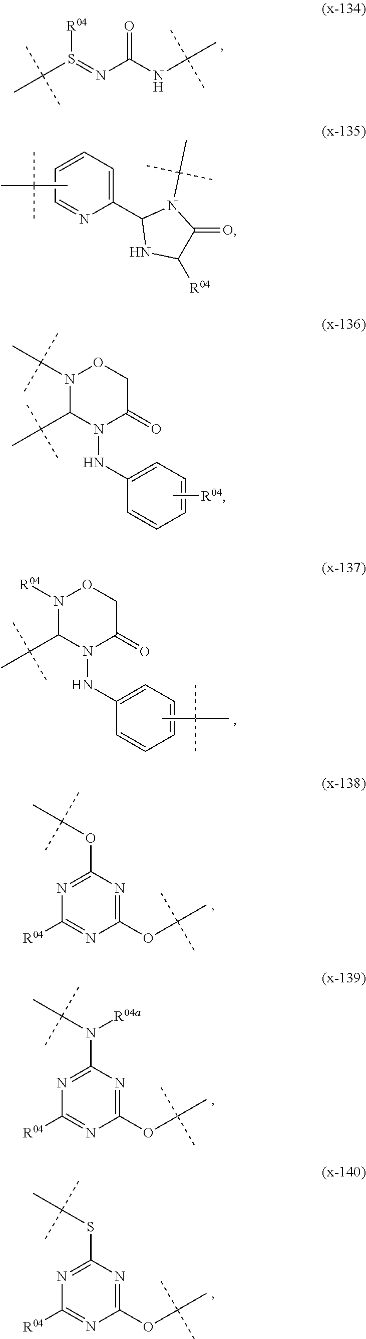

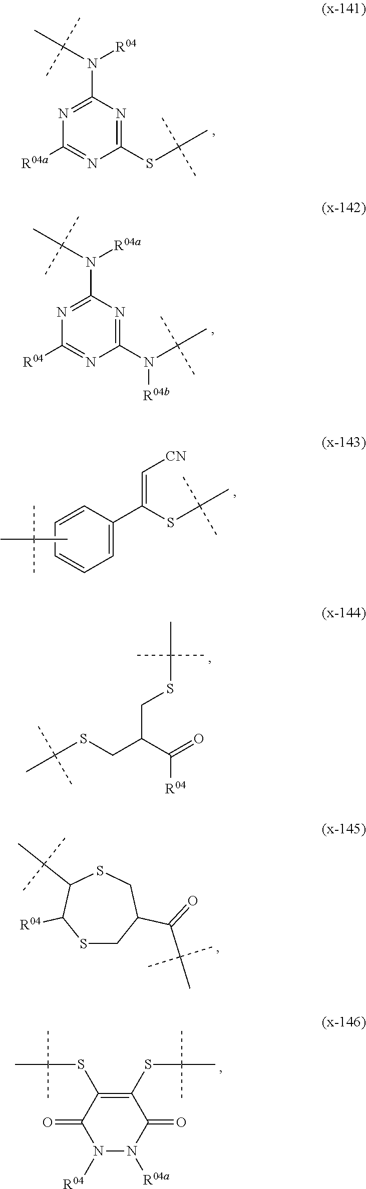

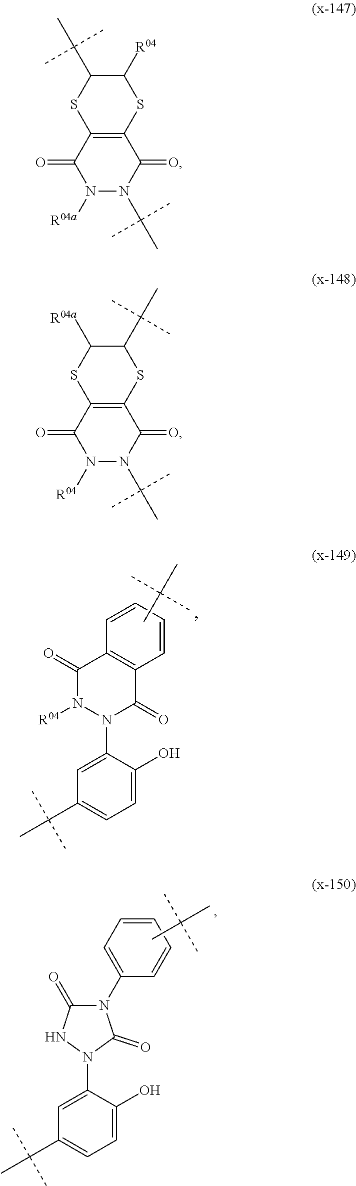

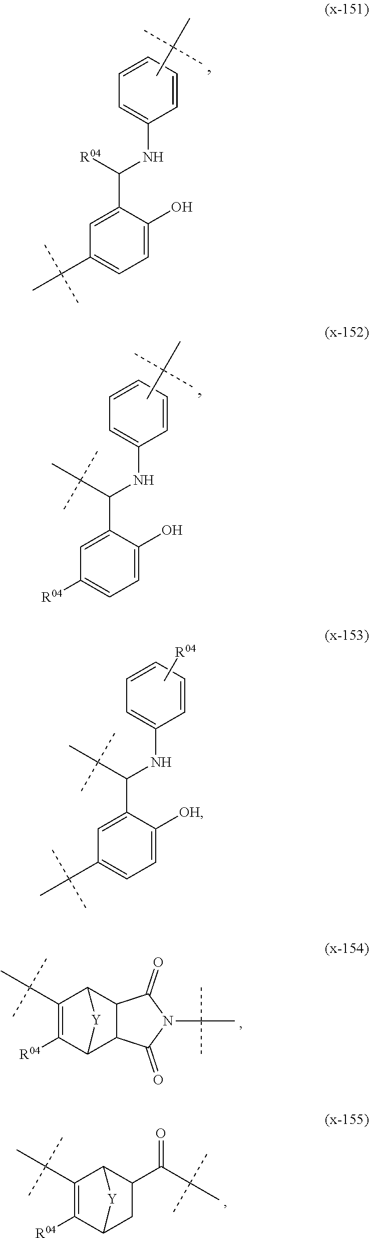

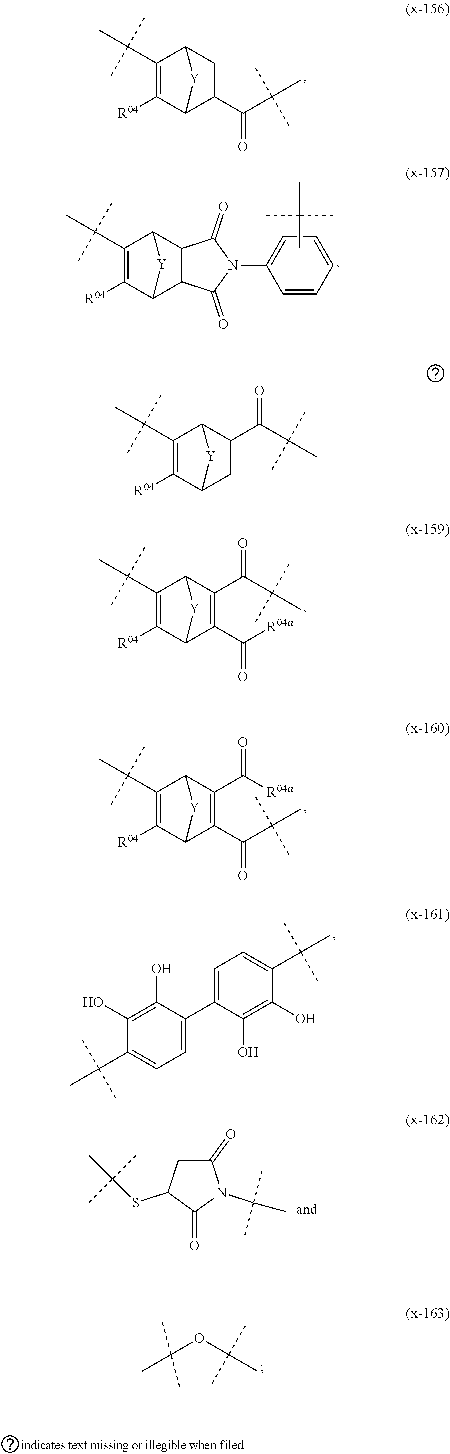

wherein
a dashed line indicates attachment to -L²- or the remainder of the moiety of formula (I-ai-i);
each —R⁰⁴, —R^{04a}, R^{04b} and —R⁰⁴, is independently selected from the group consisting of halogen, —H, —CN, -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R⁰⁶, which are the same or different, and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R⁰⁷)—, —S(O)₂N(R⁰⁷)—, —S(O)N(R⁰⁷)—, —S(O)₂—, —S(O)—, —N(R⁰⁷)S(O)₂N(R^{7a})—, —S—, —N(R⁰⁷)—, —OC(OR⁰⁷)(R^{07a})—, —N(R⁰⁷)C(O)N(R^{07a})—, and —OC(O)N(R⁰⁷)—;
each T⁰ is independently selected from the group consisting of phenyl, naphthyl, indenyl, indanyl, tetralinyl, C₃₋₁₀ cycloalkyl, 3- to 10-membered heterocyclyl, and 8-to 11-membered heterobicyclyl; wherein each T⁰ is independently optionally substituted with one or more —R⁰⁶, which are the same or different; and
each —R⁰⁶, —R⁰⁷ and —R^{07a} is independently selected from the group consisting of —H and C₁₋₆ alkyl; wherein C₁₋₆ alkyl is optionally substituted with one or more halogen, which are the same or different
each b is independently an integer selected from the group consisting of 1, 2, 3, 4, 5, 6, 7, 8,9, 10, 11, 12, 13, 14, 15 and 16;
each c is independently an integer selected from the group consisting of 1, 2, 3, 4, 5, 6, 7,8,9, 10, 11, 12, 13, 14, 15 and 16; and
n is an integer such that the molecular weight of the PEG moiety ranges from about 1 kDa to about 20 kDa.

In certain embodiments n of formula (I-ai-i) ranges from 2 to 22, i.e., n is in certain embodiments selected from the group consisting of 2, 3, 4, 5, 6, 7, 8, 9, 10, 11, 12, 13, 14, 15, 16, 17, 18, 19, 20, 21 and 22. In general, based on the number of ethylene glycol units the molecular weight of a PEG moiety can be calculated by multiplying the number of ethylene glycol units with 0.044 kDa.

In certain embodiments both moieties —X^{A}— are identical. In certain embodiments the moieties —X^{A}— are different from each other.

In certain embodiments a moiety —X^{A}— is selected from the group consisting of formula (x-1), (x-2), (x-3), (x-4), (x-5), (x-6), (x-7), (x-8), (x-9), (x-10), (x-11), (x-12), (x-13), (x-14), (x-15), (x-16), (x-17), (x-18), (x-19), (x-20), (x-21), (x-22) and (x-23). In certain embodiments a moiety —X^{A}— is selected from the group consisting of formula (x-1), (x-2), (x-3), (x-5), (x-6), (x-7), (x-8), (x-12), (x-13), (x-14), (x-15), (x-16), (x-17), (x-18), (x-19), (x-20), (x-21), (x-22) and (x-23). In certain embodiments a moiety —X^{A}— is selected from the group consisting of formula (x-1), (x-2), (x-3), (x-5), (x-6), (x-7), (x-8), (x-12), (x-13), (x-14), (x-15), (x-16), (x-17), (x-18), (x-19), (x-20) and (x-21). In certain embodiments a moiety —X^{A}— is selected from the group consisting of formula (x-1), (x-2), (x-3), (x-5), (x-12), (x-13), (x-14), (x-15), (x-16), (x-17) and (x-18).

In certain embodiments —R⁰⁴ of formula is —H. In certain embodiments —R^{04a} is —H. In certain embodiments —R^{04b} is —H. In certain embodiments —R⁰⁴, is —H.

In certain embodiments both —X^{A}— are of formula (x-1), in particular of formula (x-1′)

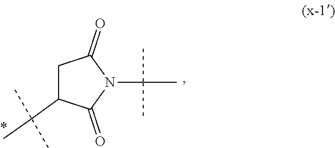

wherein the dashed line marked with the asterisk indicates attachment to -L²- and the unmarked dashed line indicates attachment to the remainder of —P—. In certain embodiments the dashed line marked with the asterisk indicates attachment to a moiety of formula (x-2) of -L²-.

In certain embodiments —P— of formula (I-a) or (I-a′) is of formula (I-aii)

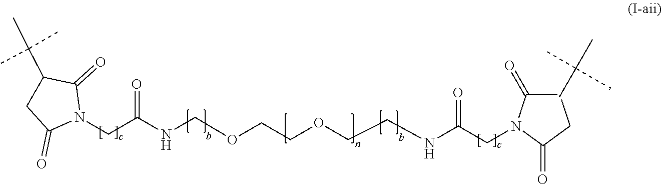

wherein
dashed lines indicate attachment to -L²-;
each b is independently an integer selected from the group consisting of 1, 2, 3, 4, 5, 6, 7, 8,9, 10, 11, 12, 13, 14, 15 and 16;
each c is independently an integer selected from the group consisting of 1, 2, 3, 4, 5, 6, 7,8,9, 10, 11, 12, 13, 14, 15 and 16; and
n is used as defined for n of formula (I-ai).

In certain embodiments —P— of formula (I-a) or (I-a′) is of formula (I-aii)

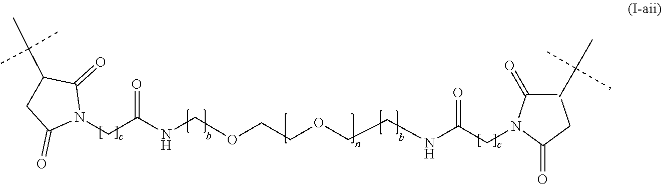

wherein
dashed lines indicate attachment to -L²-;
each b is independently an integer selected from the group consisting of 1,2, 3, 4, 5, 6, 7, 8,9, 10, 11, 12, 13, 14, 15 and 16;
each c is independently an integer selected from the group consisting of 1,2, 3, 4, 5, 6, 7, 8,9, 10, 11, 12, 13, 14, 15 and 16; and
n is used as defined for n of formula (I-ai).

In certain embodiments —P— of formula (I-a) or (I-a′) is of formula (I-aii′)

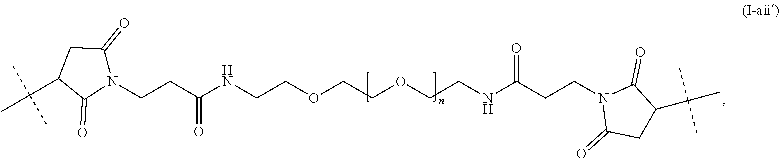

wherein
dashed lines indicate attachment to -L²-; and
n is an integer such that the molecular weight of the PEG moiety ranges from about 1 about 20 kDa.

In certain embodiments —P— of formula (I-a) or (I-a′) is selected from the group consisting of

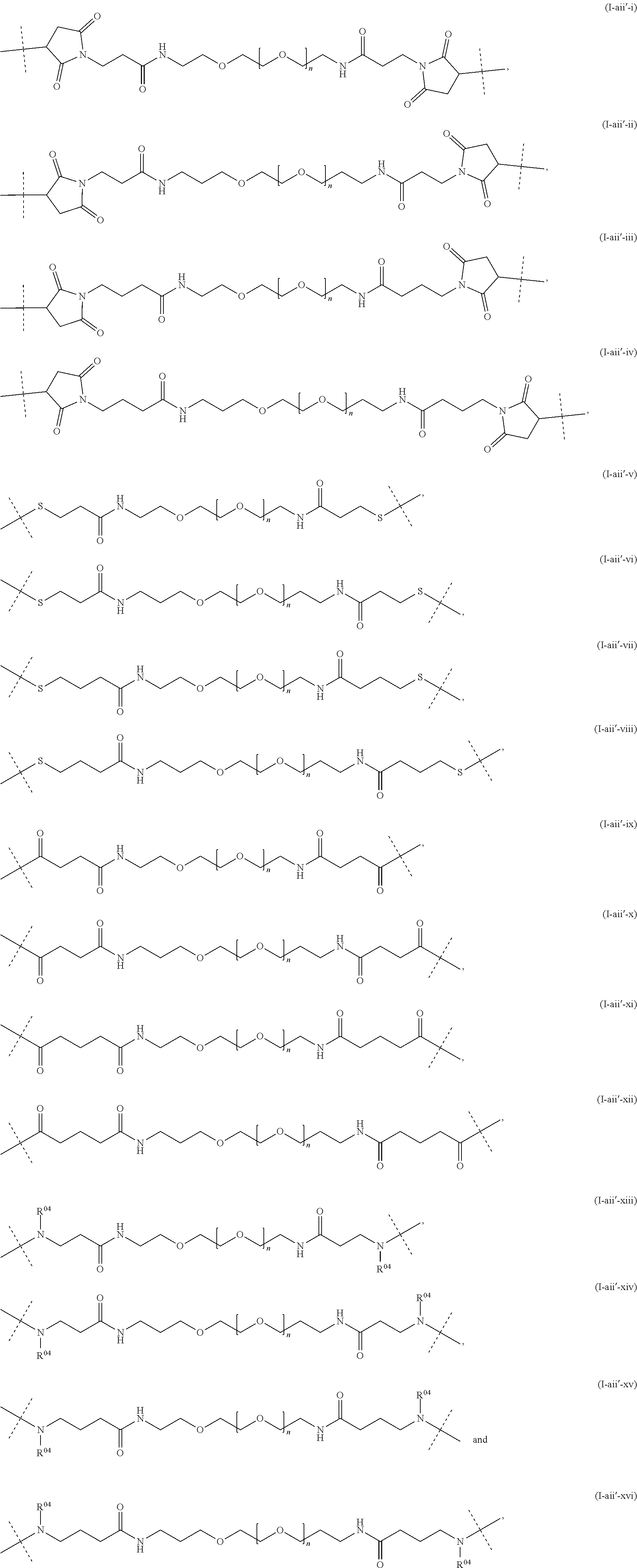

wherein
dashed lines indicate attachment to -L²-;
—R⁰⁴ is defined as above; and
n is an integer such that the molecular weight of the PEG moiety ranges from about 1 about 20 kDa.

In certain embodiments —R⁰⁴ of formula (I-aii′-xiii), (I-aii′-xiv), (I-aii′-xv) or (I-aii′-xvi) is —H.

In certain embodiments n of formulas (I-aii′-i), (I-aii′-ii), (I-aii′-iii), (I-aii′-iv), (I-aii′-v), (I-aii′vi), (I-aii′-vii), (I-aii′-viii), (I-aii′-ix), (I-aii′-x), (I-aii′-xi), (I-aii′-xii), (I-aii′-xiii), (I-aii′-xiv), (I-aii′-xv) and (I to (I-aii′-xvi) ranges from 2 to 22, i.e., n is in certain embodiments selected from the group consisting of 2, 3, 4, 5, 6, 7, 8, 9, 10, 11, 12, 13, 14, 15, 16, 17, 18, 19, 20, 32 and 22.

A dashed line in any one of formulas (I-aii′-i), (I-aii′-ii), (I-aii′-iii) and (I-aii′-vi) suitably indicates attachment to -L²- via a moiety of formula (x-2), resulting in a moiety of formula (x-162′)

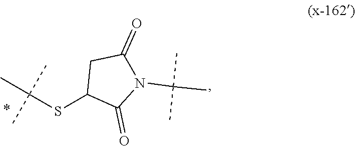

wherein the dashed line marked with the asterisk indicates attachment to the remainder of -L²- and the unmarked dashed line indicates attachment to the remainder of —P—.

A dashed line in any one of formulas (I-aii′-v), (I-aii′-vi), (I-aii′-vii) and (I-aii′-viii) suitably indicates attachment to -L²- via a moiety of formula (x-1), resulting in a moiety of formula (x-162″)

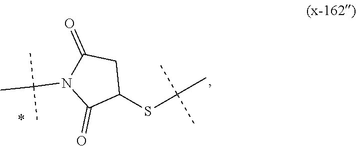

wherein the dashed line marked with the asterisk indicates attachment to the remainder of -L²- and the unmarked dashed line indicates attachment to the remainder of —P—.

A dashed line in any one of formulas (I-aii″-ix), (I-aii″-x), (I-aii″-xi) and (I-aii″-xii) suitably indicates attachment to -L²- via a moiety of formula (x-13), resulting in a moiety of formula (x-12′)

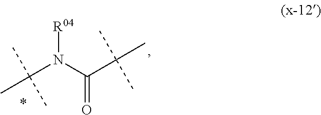

wherein the dashed line marked with the asterisk indicates attachment to the remainder of -L²- and the unmarked dashed line indicates attachment to the remainder of —P—, with —R⁰⁴ being used as defined in formula (I-ai-i). Suitably, —R⁰⁴ of formula (x-12′) is —H.

A dashed line in any one of formulas (I-aii″-xiii), (I-aii″-xiv), (I-aii″-xv) and (I-aii″-xvi) suitably indicates attachment to -L²- via a moiety of formula (x-14), resulting in a moiety of formula (x-12″)

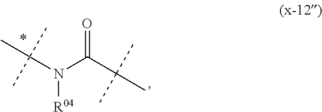

wherein the dashed line marked with the asterisk indicates attachment to the remainder of -L²- and the unmarked dashed line indicates attachment to the remainder of —P—, with —R⁰⁴ being used as defined in formula (I-ai-i). Suitably, —R⁰⁴ of formula (x-12″) is —H.

In certain embodiments n of formula (I-ai), (I-ai-i), (I-aii), (I-aii′) or (I-aii′-i) to (I-aii′-xvi) is an integer such that the molecular weight of the PEG moiety ranges from 1 to 15 kDa. In certain embodiments n of formula (I-ai), (I-ai-i), (I-aii), (I-aii′) or (I-aii′-i) to (I-aii′-xvi) is an integer such that the molecular weight of the PEG moiety ranges from 1 to 10 kDa. In certain embodiments n of formula (I-ai), (I-ai-i), (I-aii), (I-aii′) or (I-aii′-i) to (I-aii′-xvi) is an integer such that the molecular weight of the PEG moiety is about 1 kDa. In certain embodiments n of formula (I-ai), (I-ai-i), (I-aii), (I-aii′) or (I-aii′-i) to (I-aii′-xvi) is an integer such that the molecular weight of the PEG moiety is about 2 kDa. In certain embodiments n of formula (I-ai), (I-ai-i), (I-aii), (I-aii′) or (I-aii′-i) to (I-aii′-xvi) is an integer such that the molecular weight of the PEG moiety is about 2.5 kDa. In certain embodiments n of formula (I-ai), (I-ai-i), (I-aii), (I-aii′) or (I-aii′-i) to (I-aii′-xvi) is an integer such that the molecular weight of the PEG moiety is about 3 kDa. In certain embodiments n of formula (I-ai), (I-ai-i), (I-aii), (I-aii′) or (I-aii′-i) to (I-aii′-xvi) is an integer such that the molecular weight of the PEG moiety is about 4 kDa. In certain embodiments n of formula (I-ai), (I-ai-i), (I-aii), (I-aii′) or (I-aii′-i) to (I-aii′-xvi) is an integer such that the molecular weight of the PEG moiety is about 5 kDa. In certain embodiments n of formula (I-ai), (I-ai-i), (I-aii), (I-aii′) or (I-aii′-i) to (I-aii′-xvi) is an integer such that the molecular weight of the PEG moiety is about 7.5 kDa. In certain embodiments n of formula (I-ai), (I-ai-i), (I-aii), (I-aii′) or (I-aii′-i) to (I-aii′-xvi) is an integer such that the molecular weight of the PEG moiety is about 10 kDa. In certain embodiments n of formula (I-ai), (I-ai-i), (I-aii), (I-aii′) or (I-aii′-i) to (I-aii′-xvi) is an integer such that the molecular weight of the PEG moiety is about 15 kDa. In certain embodiments n of formula (I-ai), (I-ai-i), (I-aii), (I-aii′) or (I-aii′-i) to (I-aii′-xvi) is an integer such that the molecular weight of the PEG moiety is about 20 kDa.

In general, the conversion of the molecular weight of a PEG moiety to the number of ethylene glycol units, such as n in formula (I-ai), (I-ai-i), (I-aii), (I-aii′), (I-aii′-i), (I-aii′-ii), (I-aii′-iii), aii′-xii), (I-aii′-xiii), (I-aii′-xiv), (I-aii′-xv) and (I-aii′-xvi), can easily be made by dividing the molecular weight of the PEG moiety in kDa by 0.044 (molecular weight of one ethylene glycol unit: 44 Da).

In certain embodiments n of formula (I-ai), (I-ai-i), (I-aii), (I-aii′) or (I-aii′-i) to (I-aii′-xvi) is an integer ranging from and including about 22 to about 450. In certain embodiments n of formula (I-ai), (I-ai-i), (I-aii), (I-aii′) or (I-aii′-i) to (I-aii′-xvi) is about 22. In certain embodiments n of formula (I-ai), (I-ai-i), (I-aii) or (I-aii′) is about 23. In certain embodiments n of formula (I-ai), (I-ai-i), (I-aii), (I-aii′) or (I-aii′-i) to (I-aii′-xvi) is about 45. In certain embodiments n of formula (I-ai) is about 57. In certain embodiments n of formula (I-ai), (I-ai-i), (I-aii), (I-aii′) or (I-aii′-i) to (I-aii′-xvi) is about 68. In certain embodiments n of formula (I-ai), (I-ai-i), (I-aii), (I-aii′) or (I-aii′-i) to (I-aii′-xvi) is about 90. In certain embodiments n of formula (I-ai) is about 113. In certain embodiments n of formula (I-ai), (I-ai-i), (I-aii), (I-aii′) or (I-aii′-i) to (I-aii′-xvi) is about 170. In certain embodiments n of formula (I-ai), (I-ai-i), (I-aii), (I-aii′) or (I-aii′-i) to (I-aii′-xvi) is about 230. In certain embodiments n of formula (I-ai), (I-ai-i), (I-aii), (I-aii′) or (I-aii′-i) to (I-aii′-xvi) is about 340. In certain embodiments n of formula (I-ai), (I-ai-i), (I-aii), (I-aii′) or (I-aii′-i) to (I-aii′-xvi) is about 450.

In certain embodiments n of formula (I-ai), (I-ai-i), (I-aii), (I-aii′) or (I-aii′-i) to (I-aii′-xvi) is an integer ranging from and including 2 to 21. In certain embodiments n of formula (I-ai), (I-ai-i), (I-aii), (I-aii′) or (I-aii′-i) to (I-aii′-xvi) is 2. In certain embodiments n of formula (I-ai), (I-ai-i), (I-aii), (I-aii′) or (I-aii′-i) to (I-aii′-xvi) is 2. In certain embodiments n of formula (I-ai), (I-ai-i), (I-aii), (I-aii′) or (I-aii′-i) to (I-aii′-xvi) is 3. In certain embodiments n of formula (I-ai), (I-ai-i), (I-aii), (I-aii′) or (I-aii′-i) to (I-aii′-xvi) is 4. In certain embodiments n of formula (I-ai), (I-ai-i), (I-aii), (I-aii′) or (I-aii′-i) to (I-aii′-xvi) is 5. In certain embodiments n of formula (I-ai), (I-ai-i), (I-aii), (I-aii′) or (I-aii′-i) to (I-aii′-xvi) is 6. In certain embodiments n of formula (I-ai), (I-ai-i), (I-aii), (I-aii′) or (I-aii′-i) to (I-aii′-xvi) is 7. In certain embodiments n of formula (I-ai), (I-ai-i), (I-aii), (I-aii′) or (I-aii′-i) to (I-aii′-xvi) is 2.

In certain embodiments —P— of formula (I-a) or (I-a′) is of formula (P-i)

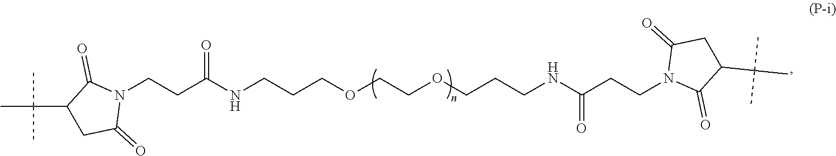

wherein
dashed lines indicate attachment to -L²-, and
n ranges from 50 to 200.

In certain embodiments —P— of formula (I-a) or (I-a′) is of formula (P-ii)

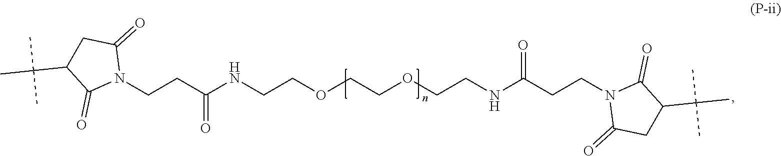

wherein
dashed lines indicate attachment to -L²-; and
n ranges from 50 to 200.

In certain embodiments n of formula (P-i) and (P-ii) ranges 60 to 180, from 70 to 160, from 80 to 160, from 90 to 140, from 95 to 130 or from 100 to 125. In certain embodiments n of formula (P-i) and (P-ii) n is selected such that P has a molecular weight of about 5 kDa.

In certain embodiments n of formulas (P-i) or (P-ii) ranges from 2 to 22, i.e., n is in certain embodiments 2, 3, 4, 5, 6, 7, 8, 9, 10, 11, 12, 13, 14, 15, 16, 17, 18, 19, 20, 21 or 22.

In certain embodiments n of formulas (P-i) or (P-ii) ranges from 23 to 49, i.e., n is in certain embodiments 23, 24, 25, 26, 27, 28, 29, 30, 31, 32, 33, 34, 35, 36, 37, 38, 39, 40, 41, 42, 43, 44, 45, 46, 47, 48 or 49.

In certain embodiments —P— of formula (I-a) or (I-a′) is of formula (I-aiv)

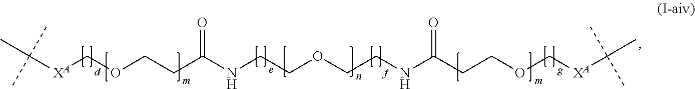

wherein
the dashed lines indicate attachment to -BP-;
each —X^{A}— is independently used as defined in formula (I-ai-i);
d, e, f and g are independently an integer selected from the group consisting of 1, 2,3, 4, 5, 6, 7, 8, 9 and 10;
each m is independently an integer ranging from 1 to 100; and
n is an integer such that the molecular weight of the PEG moiety ranges from about 1 about 20 kDa.

In certain embodiments d of formula (I-aiv) is an integer ranging from 1 to 8. In certain embodiments d of formula (I-aiv) is an integer ranging from 1 to 6. In certain embodiments d of formula (I-aiv) is 1. In certain embodiments d of formula (I-aiv) is 2. In certain embodiments d of formula (I-aiv) is 3. In certain embodiments d of formula (I-aiv) is 4. In certain embodiments d of formula (I-aiv) is 5. In certain embodiments d of formula (I-aiv) is 6.

In certain embodiments g of formula (I-aiv) is an integer ranging from 1 to 8. In certain embodiments g of formula (I-aiv) is an integer ranging from 1 to 6. In certain embodiments g of formula (I-aiv) is 1. In certain embodiments g of formula (I-aiv) is 2. In certain embodiments g of formula (I-aiv) is 3. In certain embodiments g of formula (I-aiv) is 4. In certain embodiments g of formula (I-aiv) is 5. In certain embodiments g of formula (I-aiv) is 6.

In certain embodiments d and g of formula (I-aiv) are the same integer. In certain embodiments both d and g of formula (I-aiv) are 1. In certain embodiments both d and g of formula (I-aiv) are 2. In certain embodiments both d and g of formula (I-aiv) are 3. In certain embodiments both d and g of formula (I-aiv) are 4.

In certain embodiments e of formula (I-aiv) is an integer ranging from 1 to 8. In certain embodiments e of formula (I-aiv) is an integer ranging from 1 to 6. In certain embodiments e of formula (I-aiv) is 1. In certain embodiments e of formula (I-aiv) is 2. In certain embodiments e of formula (I-aiv) is 3. In certain embodiments e of formula (I-aiv) is 4. In certain embodiments e of formula (I-aiv) is 5. In certain embodiments e of formula (I-aiv) is 6.

In certain embodiments f of formula (I-aiv) is an integer ranging from 1 to 8. In certain embodiments f of formula (I-aiv) is an integer ranging from 1 to 6. In certain embodiments f of formula (I-aiv) is 1. In certain embodiments f of formula (I-aiv) is 2. In certain embodiments f of formula (I-aiv) is 3. In certain embodiments f of formula (I-aiv) is 4. In certain embodiments f of formula (I-aiv) is 5. In certain embodiments f of formula (I-aiv) is 6.

In certain embodiments e and f of formula (I-aiv) are the same integer. In certain embodiments both e and f of formula (I-aiv) are 1. In certain embodiments both e and f of formula (I-aiv) are 2. In certain embodiments both e and f of formula (I-aiv) are 3. In certain embodiments both e and f of formula (I-aiv) are 4.

In certain embodiments n of formula (I-aiv) is an integer such that the molecular weight of the PEG moiety ranges from 1 to 15 kDa. In certain embodiments n of formula (I-aiv) is an integer such that the molecular weight of the PEG moiety ranges from 1 to 10 kDa. In certain embodiments n of formula (I-aiv) is an integer such that the molecular weight of the PEG moiety is about 1 kDa. In certain embodiments n of formula (I-aiv) is an integer such that the molecular weight of the PEG moiety is about 2 kDa. In certain embodiments n of formula (I-aiv) is an integer such that the molecular weight of the PEG moiety is about 2.5 kDa. In certain embodiments n of formula (I-aiv) is an integer such that the molecular weight of the PEG moiety is about 3 kDa. In certain embodiments n of formula (I-aiv) is an integer such that the molecular weight of the PEG moiety is about 4 kDa. In certain embodiments n of formula (I-aiv) is an integer such that the molecular weight of the PEG moiety is about 5 kDa. In certain embodiments n of formula (I-aiv) is an integer such that the molecular weight of the PEG moiety is about 7.5 kDa. In certain embodiments n of formula (I-aiv) is an integer such that the molecular weight of the PEG moiety is about 10 kDa. In certain embodiments n of formula (I-aiv) is an integer such that the molecular weight of the PEG moiety is about 15 kDa. In certain embodiments n of formula (I-aiv) is an integer such that the molecular weight of the PEG moiety is about 20 kDa.

In certain embodiments n of formula (I-aiv) is an integer ranging from and including about 22 to about 450. In certain embodiments n of formula (I-aiv) is about 22. In certain embodiments n of formula (I-aiv) is about 23. In certain embodiments n of formula (I-aiv) is about 45. In certain embodiments n of formula (I-ai) is about 57. In certain embodiments n of formula (I-aiv) is about 68. In certain embodiments n of formula (I-aiv) is about 90. In certain embodiments n of formula (I-ai) is about 113. In certain embodiments n of formula (I-aiv) is about 170. In certain embodiments n of formula (I-aiv) is about 230. In certain embodiments n of formula (I-aiv) is about 340. In certain embodiments n of formula (I-aiv) is about 450.

In certain embodiments the at least one polymeric moiety is a linear polymeric moiety —P— comprising a first and a second end and the at least two moieties of formula (I) and/or the at least one moiety of formula (I′) is/are conjugated to —P— at sites selected from the group consisting of internal sites, the first end and the second end.

In certain embodiments the at least one polymeric moiety of the compounds of the present invention comprises a plurality of linearly connected units selected from the group consisting of

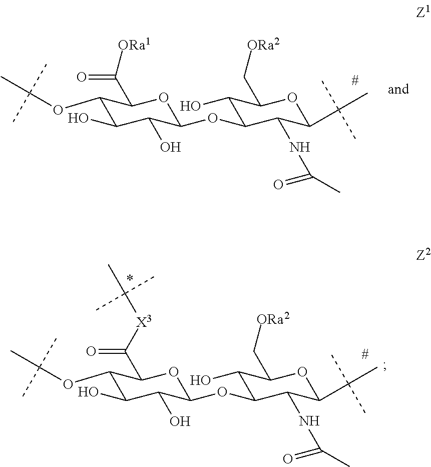

wherein
an unmarked dashed line indicates a point of attachment to an adjacent unit at a dashed line marked with # or to a hydrogen,
a dashed line marked with # indicates a point of attachment to an adjacent unit at an unmarked dashed line or to a hydroxyl;
a dashed line marked with the asterisk indicates attachment to -L²-;
the total number of units Z² in —P— is at least 2;
the total number of units Z¹ and Z² is at least 5;
—Ra¹ and —Ra² are each independently selected from the group consisting of hydrogen; C₁₋₄ alkyl; an alkali metal ion, an ammonium ion, an alkaline earth metal ion, or other suitable counterion;
—X³— is selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR³)(R^{y3a})—, —N(R^{y3})C(O)N(R^{y3a})—, and —OC(O)N(R^{y3})—;
—R^{y1} and —R^{y1a} are independently of each other selected from the group consisting of —H, -T, C₁₋₅₀ alkyl, C₂₋₅₀ alkenyl, and C₂₋₅₀ alkynyl; wherein -T, C₁₋₅₀ alkyl, C₂₋₅₀ alkenyl, and C₂₋₅₀ alkynyl are optionally substituted with one or more —R^{y2}, which are the same or different, and wherein C₁₋₅ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR⁴)(R^{y4a})—, —N(R^{y4})C(O)N(R^{y4a})—, and —OC(O)N(R^{y4})—;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;
each —R^{y2} is independently selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and
each —R^{y3}, —R^{y3a}, —R^{y4}, —R^{y4a}, —R^{y5}, —R^{y5a} and —R^{y5b} is independently selected from the group consisting of —H, and C₁₋₆ alkyl, wherein C₁₋₆ alkyl is optionally substituted with one or more halogen, which are the same or different.

In certain embodiments —X³— is selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₂₀ alkyl, C₂₋₂₀ alkenyl, and C₂₋₂₀ alkynyl are optionally substituted with one or more —R^{y2}, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;
—R^{y1} and —R^{y1a} are independently of each other selected from the group consisting of —H, -T, C₁₋₁₀ alkyl, C₂₋₁₀ alkenyl, and C₂₋₁₀ alkynyl; wherein -T, C₁₋₁₀ alkyl, C₂₋₁₀ alkenyl, and C₂₋₁₀ alkynyl are optionally substituted with one or more —R^{y2}, which are the same or different, and wherein C₁₋₁₀ alkyl, C₂₋₁₀ alkenyl, and C₂₋₁₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR⁴)(R^{y4a})—, —N(R^{y4})C(O)N(R^{y4a})—, and —OC(O)N(R^{y4})—;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;
—R^{y2} is selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)O R^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and
each —R^{y3}, —R^{y3a}, —R^{y4}, —R^{y4a}, —R^{y5}, —R^{y5a} and —R^{y5b} is independently of each other selected from the group consisting of —H, and C₁₋₆ alkyl; wherein C₁₋₆ alkyl is optionally substituted with one or more halogen, which are the same or different.

In certain embodiments —X³— is selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR³)(R^{y3a})—, —N(R^{y3})C(O)N(R^{y3a})—, and —OC(O)N(R^{y3})—;
—R^{y1} and —R^{y1a} are independently selected from the group consisting of —H, -T, C₁₋₁₀ alkyl, C₂₋₁₀ alkenyl, and C₂₋₁₀ alkynyl;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl;
each —R^{y2} is independently selected from the group consisting of halogen, and C₁₋₆ alkyl; and each —R^{y3}, —R^{y3a}, —R^{y4}, —R^{y4a}, —R^{y5}, —R^{y1a} and —R^{y5b} is independently of each other selected from the group consisting of —H, and C₁₋₆ alkyl; wherein C₁₋₆ alkyl is optionally substituted with one or more halogen, which are the same or different.

In certain embodiments the total number of units Z¹ and Z² is such that —P— has a molecular weight ranging from about 1 about 20 kDa. In certain embodiments the total number of units Z¹ and Z² is such that —P— has a molecular weight ranging from about 1 about 15 kDa. In certain embodiments the total number of units Z¹ and Z² is such that —P— has a molecular weight of about 1 kDa. In certain embodiments the total number of units Z¹ and Z² is such that —P— has a molecular weight of about 2 kDa. In certain embodiments the total number of units Z¹ and Z² is such that —P— has a molecular weight of about 3 kDa. In certain embodiments the total number of units Z¹ and Z² is such that —P— has a molecular weight of about 4 kDa. In certain embodiments the total number of units Z¹ and Z² is such that —P— has a molecular weight of about 5 kDa. In certain embodiments the total number of units Z¹ and Z² is such that —P— has a molecular weight of about 7.5 kDa. In certain embodiments the total number of units Z¹ and Z² is such that —P— has a molecular weight of about 10 kDa. In certain embodiments the total number of units Z¹ and Z² is such that —P— has a molecular weight of about 15 kDa. In certain embodiments the total number of units Z¹ and Z² is such that —P— has a molecular weight of about 20 kDa.

In certain embodiments —X³— is of formula (I-b)

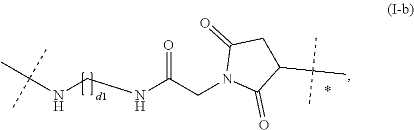

wherein
the unmarked dashed line indicates attachment to the carbonyl of Z²;
the dashed line marked with the asterisk indicates attachment to -L²-;
d1 is independently an integer selected from the group consisting of 1,2, 3, 4, 5, 6, 7, 8, 9, 10, 11, 12, 13, 14, 15 and 16;
d2 is independently an integer selected from the group consisting of 1,2, 3, 4, 5, 6, 7, 8, 9, 10, 11, 12, 13, 14, 15 and 16.

In certain embodiments d1 of formula (I-b) is 1. In certain embodiments d1 of formula (I-b) is 2. In certain embodiments d1 of formula (I-b) is 3. In certain embodiments d1 of formula (I-b) is 4. In certain embodiments d1 of formula (I-b) is 5. In certain embodiments d1 of formula (I-b) is 6.

In certain embodiments d2 of formula (I-b) is 1. In certain embodiments d2 of formula (I-b) is 2. In certain embodiments d2 of formula (I-b) is 3. In certain embodiments d2 of formula (I-b) is 4. In certain embodiments d2 of formula (I-b) is 5. In certain embodiments d2 of formula (I-b) is 6.

In certain embodiments —X³— is of formula (I-b′)

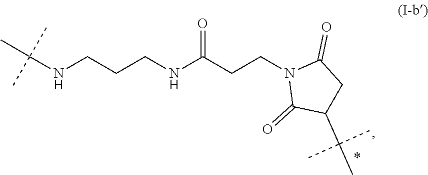

wherein
the unmarked dashed line indicates attachment to the carbonyl of Z²; and
the dashed line marked with the asterisk indicates attachment to -L²-.

In certain embodiments the compound is of formula (I-c) or (I-c′)

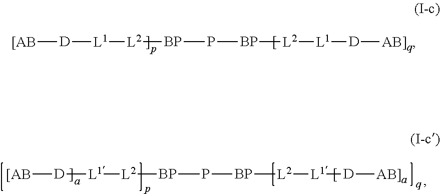

wherein
each -L²- is independently a spacer moiety or is absent;
each -L¹- is independently a linker moiety that is covalently and reversibly conjugated to -D-;
each -L^{1′}- is independently a linker moiety that is covalently and reversibly conjugated to -D-;
each -D- is independently a drug moiety;
each -AB is independently an albumin-binding moiety;
—P— is a polymeric moiety;
each BP is independently a branching point;
each a is independently an integer selected from the group consisting of 1, 2, 3, 4, 5, 6, 7,8,9, 10, 11, 12, 13, 14, 15 and 16; and
p and q are independently an integer selected from the group consisting of 2, 3, 4, 5, 6, 7, 8,9, 10, 11, 12, 13, 14, 15 and 16.

In certain embodiments the compound is of formula (I-c). In certain embodiments the compound is of formula (I-c′).

It is understood that a moiety -L^{1′}- is conjugated to more than one moiety -D and that all linkages between -L^{1′}- and a moiety -D- are covalent and reversible.

In certain embodiments both a of formula (I-c′) are the same integer. In certain embodiments both a of formula (I-c′) are 2. In certain embodiments both a of formula (I-c′) are 3. In certain embodiments both a of formula (I-c′) are 4. In certain embodiments both a of formula (I-c′) are 5. In certain embodiments both a of formula (I-a′) are 6. In certain embodiments both a of formula (I-c′) are 7. In certain embodiments both a of formula (I-c′) are 8. In certain embodiments both a of formula (I-c′) are 9. In certain embodiments both a of formula (I-c′) are 10. In certain embodiments both a of formula (I-c′) are 11. In certain embodiments both a of formula (I-c′) are 12. In certain embodiments both a of formula (I-c′) are 13. In certain embodiments both a of formula (I-c′) are 14. In certain embodiments both a of formula (I-c′) are 15. In certain embodiments both a of formula (I-c′) are 16.

In certain embodiments p and q of formula (I-c) or (I-c′) are the same integer. In certain embodiments p and q of formula (I-c) or (I-c′) are a different integer.

Embodiments for —P— are as described for formula (I-a) and (I-a′) elsewhere herein.

Each BP of formula (I-c) and (I-c′) is independently a branching point. In certain embodiments both BP are identical. In certain embodiments both BP are different. BP point may be a single atom, such as a nitrogen or carbon atom, or may be a group of atoms, such as a di-, tri- or tetraamino acid. A diamino acid may be selected from the group consisting of lysine, ornithine, 2,3-diaminoproprionic acid and 2,4-diaminobutyric acid, each either in R- and S-configuration.

In certain embodiments BP of formula (I-c′) comprises a lysine, in particular a lysine in S-configuration. In certain moieties both moieties BP of formula (I-c′) comprise a lysine, in particular a lysine in S-configuration.

In certain embodiments both moeities BP have the structure of formula (I-c-i)

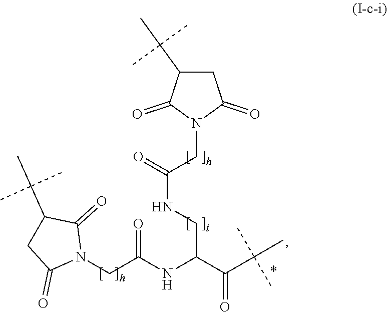

wherein
the unmarked dashed lines indicate attachment to -L²_;
the dashed line marked with the asterisk indicates attachment to —P—; and
each h is independently an integer selected from the group consisting of 1, 2, 3, 4, 5, 6, 7, 8, 9 and 10; and
each i is independently an integer selected from the group consisting of 1, 2, 3, 4, 5, 6, 7, 8, 9 and 10.

In certain embodiments both h of formula (I-c-i) are the same integer. In certain embodiments both h of formula (I-c-i) are 2 and i of formula (I-c-i) is 4. In certain embodiments both h of formula (I-c-i) are 3 and i of formula (I-c-i) is 4. In certain embodiments both h of formula (I-c-i) are 4 and i of formula (I-c-i) is 4.

In certain embodiments both moieties BP have the structure of formula (I-c-i′)

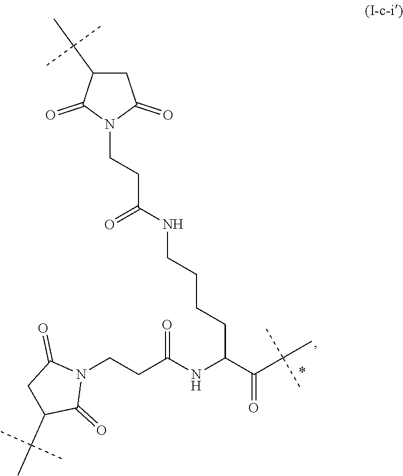

wherein
the unmarked dashed lines indicate attachment to -L²-; and
the dashed line marked with the asterisk indicates attachment to —P—.

In certain embodiments one BP of formula (I-c′) is lysine, in particular lysine in S configuration. In certain embodiments both moeities BP of formula (I-c′) are lysine, in particular lysine in S configuration.

In certain embodiments the moiety BP—P—BP is of formula (I-d′)

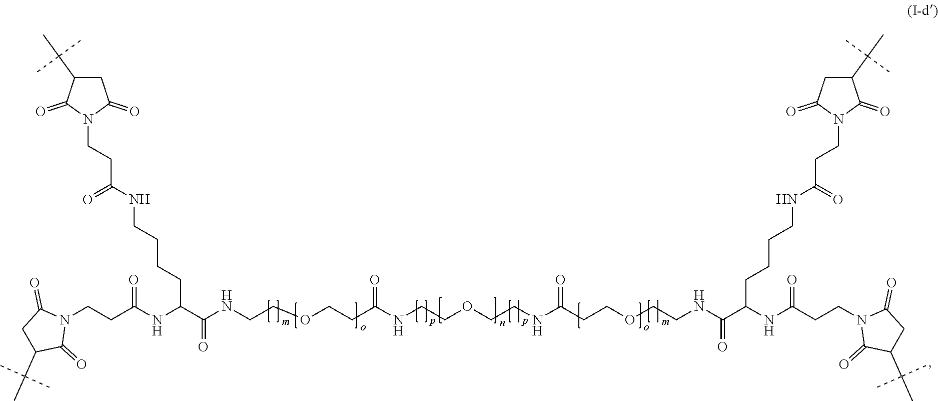

wherein
the dashed lines indicate attachment to -L²-;
each m is independently an integer selected from the group consisting of 1, 2, 3, 4, 5, 6, 7 and 8;
n is an integer ranging from 2 to 200;
each o is independently an integer selected from the group consisting of 1, 2, 3, 4, 5, 6, 7 and 8; and
each p is independently an integer selected from the group consisting of 1, 2, 3, 4, 5, 6, 7 and 8.

In certain embodiments the moiety BP—P—BP is of formula (I-d)

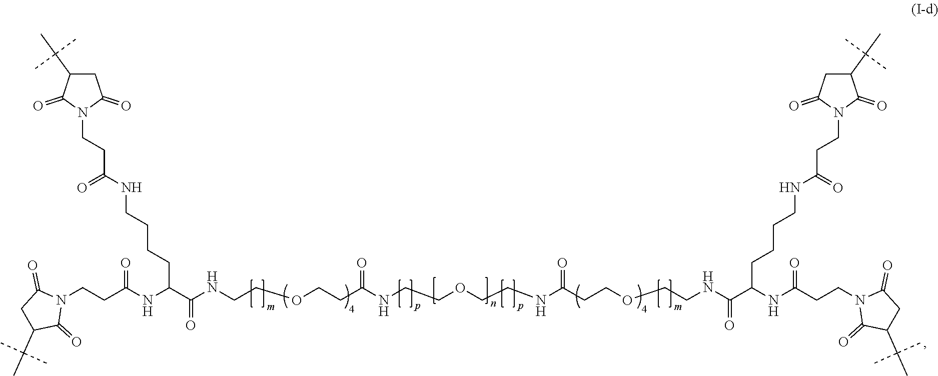

wherein
the dashed lines indicate attachment to -L²-;
each m is independently an integer selected from the group consisting of 1, 2, 3, 4, 5, 6, 7 and 8;
each p is independently an integer selected from the group consisting of 1, 2, 3, 4, 5, 6, 7 and 8; and
n is an integer ranging from 2 to 200.

In certain embodiments n of formula (I-d) and (I-d′) ranges 60 to 180, from 70 to 160, from 80 to 160, from 90 to 140, from 95 to 130 or from 100 to 125. In certain embodiments n of formula (I-d) and (I-d′) is selected such that P has a molecular weight of about 5 kDa.

In certain embodiments the moiety BP—P—BP is of formula (I-e)

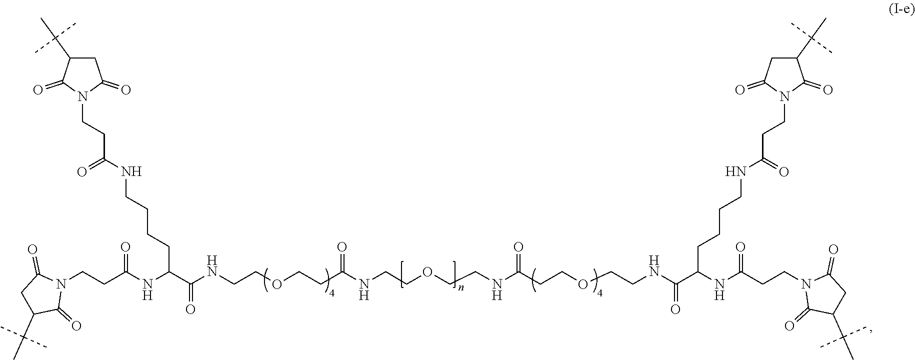

wherein dashed lines indicate attachment to -L²-; and
n is an integer ranging from 2 to 200.

In certain embodiments n of formula (I-e) ranges 60 to 180, from 70 to 160, from 80 to 160, from 90 to 140, from 95 to 130 or from 100 to 125. In certain embodiments n of formula (I-e) is selected such that P has a molecular weight of about 5 kDa.

In certain embodiments the at least one polymeric moiety of the compound of the present invention is a multi-arm polymeric moiety. Such multi-arm polymeric moiety comprises at least one branching point. Accordingly, in certain embodiments the compound comprises at least one branching point B, to which at least two moieties of formula (Ib) and/or at least one moiety of formula (Ib′) are conjugated, wherein formula (Ib) and (Ib′) are

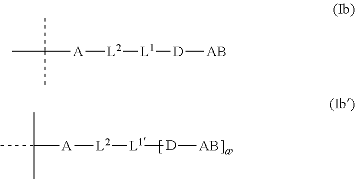

wherein
the dashed line indicates attachment to a branching point B;
-A- is a polymeric moiety
each -L²- is independently a spacer moiety or is absent;
each -L¹- is independently a linker moiety that is covalently and reversibly conjugated to -D-;
each -L^{1′}- is independently a linker moiety that is covalently and reversibly conjugated to -D-;
each -D- is independently a drug moiety;
each -AB is independently an albumin-binding moiety; and
each a is independently an integer selected from the group consisting of 1, 2, 3, 4, 5, 6, 7, 8,9, 10, 11, 12, 13, 14, 15 and 16.

In certain embodiments the compound comprises at least one branching point B, to which at least two moieties of formula (Ib) are conjugated, wherein formula (Ib) is

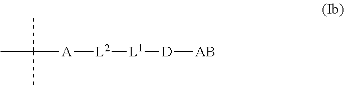

wherein
the dashed line indicates attachment to a branching point B;
-A- is a polymeric moiety
each -L²- is independently a spacer moiety or is absent;
each -L¹- is independently a linker moiety that is covalently and reversibly conjugated to -D-;
each -D- is independently a drug moiety; and
each -AB is independently an albumin-binding moiety.

In certain embodiments the compound comprises at least one branching point B, to which at least one moiety of formula (Ib′) is conjugated, wherein formula (Ib′) is

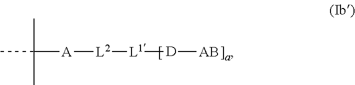

wherein
the dashed line indicates attachment to a branching point B;
-A- is a polymeric moiety
each -L²- is independently a spacer moiety or is absent;
each -L^{1′}- is independently a linker moiety that is covalently and reversibly conjugated to -D-;
each -D- is independently a drug moiety;
each -AB is independently an albumin-binding moiety; and
each a is independently an integer selected from the group consisting of 1, 2, 3, 4, 5, 6, 7, 8,9, 10, 11, 12, 13, 14, 15 and 16.

The moiety -A- of formula (Ib) or (Ib′) comprises one or more polymer, such as a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof.

In certain embodiments -A- of formula (Ib) or (Ib′) comprises a PEG-based polymer or a hyaluronic acid-based polymer. In certain embodiments -A- of formula (Ib) or (Ib′) comprises a PEG-based polymer. In certain embodiments -A- of formula (Ib) or (Ib′) comprises a hyaluronic acid-based polymer.

In certain embodiments -A- of formula (Ib) or (Ib′) is of formula (I-ba)

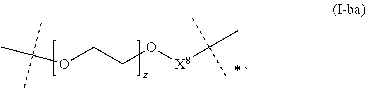

wherein
the unmarked dashed line indicates attachment to the branching point B;
the dashed line marked with the asterisk indicates attachment to -L²-;
z is an integer selected such that the molecular weight of -A- ranges from about 0.5 kDa to about 10 kDa; —X⁸— is selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR³)(R^{y3a})—, —N(R^{y3})C(O)N(R^{y3a})—, and —OC(O)N(R³)—;
—R^{y1} and —R^{y1a} are independently of each other selected from the group consisting of —H, -T, C₁₋₅₀ alkyl, C₂₋₅₀ alkenyl, and C₂₋₅₀ alkynyl; wherein -T, C₁₋₅₀ alkyl, C₂₋₅₀ alkenyl, and C₂₋₅₀ alkynyl are optionally substituted with one or more —R^{y2}, which are the same or different, and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR⁴)(R^{y4a})—, —N(R^{y4})C(O)N(R^{y4a})—, and —OC(O)N(R^{y4})—;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;
each —R^{y2} is independently selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —N O₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and
each —R^{y3}, —R^{y3a}, —R^{y4}, —R^{y4a}, —R^{y5}, —R^{y5a} and —R^{y5b} is independently selected from the group consisting of —H, and C₁₋₆ alkyl, wherein C₁₋₆ alkyl is optionally substituted with one or more halogen, which are the same or different.

In certain embodiments —X⁸- is selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₂₀ alkyl, C₂₋₂₀ alkenyl, and C₂₋₂₀ alkynyl are optionally substituted with one or more —R^{y2}, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR³)(R^{y3a})—, —N(R^{y3})C(O)N(R^{y3a})—, and —OC(O)N(R^{y3})—;
—R^{y1} and —R^{y1a} are independently of each other selected from the group consisting of —H, -T, C₁₋₁₀ alkyl, C₂₋₁₀ alkenyl, and C₂₋₁₀ alkynyl; wherein -T, C₁₋₁₀ alkyl, C₂₋₁₀ alkenyl, and C₂₋₁₀ alkynyl are optionally substituted with one or more —R^{y2}, which are the same or different, and wherein C₁₋₁₀ alkyl, C₂₋₁₀ alkenyl, and C₂₋₁₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR⁴)(R^{y4a})—, —N(R^{y4})C(O)N(R^{y4a})—, and —OC(O)N(R^{y4})—;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;
—R^{y2} is selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and
each —R^{y3}, —R^{y3a}, —R^{y4}, —R^{y4a}, —R^{y5}, —R^{y5a} and —R^{y5b} is independently of each other selected from the group consisting of —H, and C₁₋₆ alkyl; wherein C₁₋₆ alkyl is optionally substituted with one or more halogen, which are the same or different.

In certain embodiments —X⁸— is selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —N(R^{y3})—, —OC(OR^{y3})(R^{y3a})—, —N(R^{y3})C(O)N(R^{y3a})—, and —OC(O)N(R^{y3})—;
—R^{y1} and —R^{y1a} are independently selected from the group consisting of —H, -T, C₁₋₁₀ alkyl, C₂₋₁₀ alkenyl, and C₂₋₁₀ alkynyl;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl;
each —R^{y2} is independently selected from the group consisting of halogen, and C₁₋₆ alkyl; and each —R^{y3}, —R^{y3a}, —R^{y4}, —R^{y4a}, —R⁵, —R^{y5a} and —R^{y5b} is independently of each other selected from the group consisting of —H, and C₁₋₆ alkyl; wherein C₁₋₆ alkyl is optionally substituted with one or more halogen, which are the same or different.

In certain embodiments -A- of formula (Ib) or (Ib′) is of formula (I-bb) or (I-bb′)

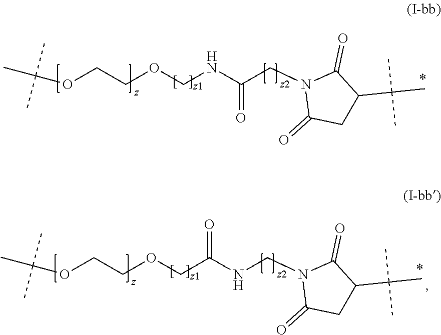

wherein
the unmarked dashed line indicates attachment to the branching point B;
the dashed line marked with the asterisk indicates attachment to -L²-;
z is an integer selected such that the molecular weight of -A- ranges from about 0.5 kDa to about 10 kDa;
z1 is selected from the group consisting of 1, 2, 3, 4, 5, 6, 7, 8, 9, 10, 11, 12, 13, 14, 15 and 16; and
z2 is selected from the group consisting of 1, 2, 3, 4, 5, 6, 7, 8, 9, 10, 11, 12, 13, 14, 15 and 16.

In certain embodiments -A- of formula (Ib) or (Ib′) is of formula (I-bb). In certain embodiments -A- of formula (Ib) or (Ib′) is of formula (I-bb′).

In certain embodiments -A- of formula (Ib) or (Ib′) is of formula (I-bc) or (I-bc′)

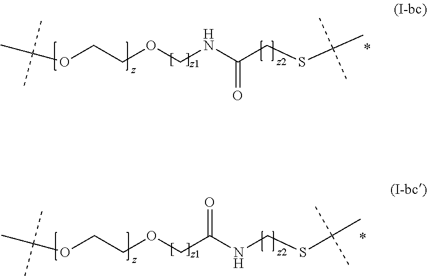

wherein
the unmarked dashed line indicates attachment to the branching point B;
the dashed line marked with the asterisk indicates attachment to -L²-;
z is an integer selected such that the molecular weight of -A- ranges from about 0.5 kDa to about 10 kDa;
z1 is selected from the group consisting of 1, 2, 3, 4, 5, 6, 7, 8, 9, 10, 11, 12, 13, 14, 15 and 16; and
z2 is selected from the group consisting of 1, 2, 3, 4, 5, 6, 7, 8, 9, 10, 11, 12, 13, 14, 15 and 16.

In certain embodiments -A- of formula (Ib) or (Ib′) is of formula (I-bc). In certain embodiments -A- of formula (Ib) or (Ib′) is of formula (I-bc′).

In certain embodiments z1 of formula (I-bb), (I-bb′), (I-bc) or (I-bc′) is 1. In certain embodiments z1 of formula (I-bb), (I-bb′), (I-bc) or (I-bc′) is 2. In certain embodiments z1 of formula (I-bb), (I-bb′), (I-bc) or (I-bc′) is 3. In certain embodiments z1 of formula (I-bb), (I-bb′), (I-bc) or (I-bc′) is 4. In certain embodiments z1 of formula (I-bb), (I-bb′), (I-bc) or (I-bc) is 5. In certain embodiments z1 of formula (I-bb), (I-bb′), (I-bc) or (I-bc′) is 6. In certain embodiments z1 of formula (I-bb), (I-bb′), (I-bc) or (I-bc′) is 7. In certain embodiments z1 of formula (I-bb), (I-bb′), (I-bc) or (I-bc) is 8. In certain embodiments z1 of formula (I-bb), (I-bb′), (I-bc) or (I-bc′) is 9. In certain embodiments z1 of formula (I-bb), (I-bb′), (I-bc) or (I-bc′) is 10. In certain embodiments z1 of formula (I-bb), (I-bb′), (I-bc) or (I-bc′) is 11. In certain embodiments z1 of formula (I-bb), (I-bb′), (I-bc) or (I-bc′) is 12. In certain embodiments z1 of formula (I-bb), (I-bb′), (I-bc) or (I-bc′) is 13. In certain embodiments z1 of formula (I-bb), (I-bb′), (I-bc) or (I-bc′) is 14. In certain embodiments z1 of formula (I-bb), (I-bb′), (I-bc) or (I-bc′) is 15. In certain embodiments z1 of formula (I-bb), (I-bb′), (I-bc) or (I-bc′) is 16.

In certain embodiments z2 of formula (I-bb), (I-bb′), (I-bc) or (I-bc′) is 1. In certain embodiments z2 of formula (I-bb), (I-bb′), (I-bc) or (I-bc′) is 2. In certain embodiments z2 of formula (I-bb), (I-bb′), (I-bc) or (I-bc′) is 3. In certain embodiments z2 of formula (I-bb), (I-bb′), (I-bc) or (I-bc′) is 4. In certain embodiments z2 of formula (I-bb), (I-bb′), (I-bc) or (I-bc′) is 5. In certain embodiments z2 of formula (I-bb), (I-bb′), (I-bc) or (I-bc′) is 6. In certain embodiments z2 of formula (I-bb), (I-bb′), (I-bc) or (I-bc′) is 7. In certain embodiments z2 of formula (I-bb), (I-bb′), (I-bc) or (I-bc′) is 8. In certain embodiments z2 of formula (I-bb), (I-bb′), (I-bc) or (I-bc′) is 9. In certain embodiments z2 of formula (I-bb), (I-bb′), (I-bc) or (I-bc′) is 10. In certain embodiments z2 of formula (I-bb), (I-bb′), (I-bc) or (I-bc′) is 11. In certain embodiments z2 of formula (I-bb), (I-bb′), (I-bc) or (I-bc′) is 12. In certain embodiments z2 of formula (I-bb), (I-bb′), (I-bc) or (I-bc′) is 13. In certain embodiments z2 of formula (I-bb), (I-bb′), (I-bc) or (I-bc′) is 14. In certain embodiments z2 of formula (I-bb), (I-bb′), (I-bc) or (I-bc′) is 15. In certain embodiments z2 of formula (I-bb), (I-bb′), (I-bc) or (I-bc′) is 16.

In certain embodiments z1 of formula (I-bb), (I-bb′), (I-bc) or (I-bc′) is 3 and z2 of formula (I-bb), (I-bb′), (I-bc) or (I-bc′) is 2. In certain embodiments z1 of formula (I-bb), (I-bb′), (I-bc) or (I-bc′) is 2 and z2 of formula (I-bb), (I-bb′), (I-bc) or (I-bc′) is 2.

In certain embodiments z of formula (I-ba), (I-bb), (I-bb′), (I-bc) or (I-bc′) is an integer such that the molecular weight of the -A- ranges from and includes about 0.5 kDa to about 10 kDa. In certain embodiments z of formula (I-ba), (I-bb), (I-bb′), (I-bc) or (I-bc′) is an integer such that the molecular weight of the -A- is about 0.5 kDa. In certain embodiments z of formula (I-ba), (I-bb), (I-bb′), (I-bc) or (I-bc′) is an integer such that the molecular weight of the -A- is about 1 kDa. In certain embodiments of formula (I-ba), (I-bb), (I-bb′), (I-bc) or (I-bc′) is an integer such that the molecular weight of the -A- is about 1.5 kDa. In certain embodiments z of formula (I-ba), (I-bb), (I-bb′), (I-bc) or (I-bc′) is an integer such that the molecular weight of the -A- is about 2 kDa. In certain embodiments z of formula (I-ba), (I-bb), (I-bb′), (I-bc) or (I-bc′) is an integer such that the molecular weight of the -A- is about 2.5 kDa. In certain embodiments z of formula (I-ba), (I-bb), (I-bb′), (I-bc) or (I-bc′) is an integer such that the molecular weight of the -A- is about 3 kDa. In certain embodiments z of formula (I-ba), (I-bb), (I-bb′), (I-bc) or (I-bc′) is an integer such that the molecular weight of the -A- is about 3.5 kDa.

In certain embodiments z of formula (I-ba), (I-bb), (I-bb′), (I-bc) or (I-bc′) is an integer such that the molecular weight of the -A- is about 4 kDa. In certain embodiments z of formula (I-ba), (I-bb), (I-bb′), (I-bc) or (I-bc′) is an integer such that the molecular weight of the -A- is about 5 kDa. In certain embodiments z of formula (I-ba), (I-bb), (I-bb′), (I-bc) or (I-bc′) is an integer such that the molecular weight of the -A- is about 6 kDa. In certain embodiments z of formula (I-ba), (I-bb), (I-bb′), (I-bc) or (I-bc′) is an integer such that the molecular weight of the -A- is about 7 kDa. In certain embodiments of formula (I-ba), (I-bb), (I-bb′), (I-bc) or (I-bc′) is an integer such that the molecular weight of the -A- is about 8 kDa. In certain embodiments z of formula (I-ba), (I-bb), (I-bb′), (I-bc) or (I-bc′) is an integer such that the molecular weight of the -A- is about 9 kDa. In certain embodiments z of formula (I-ba), (I-bb), (I-bb′), (I-bc) or (I-bc′) is an integer such that the molecular weight of the -A- is about 10 kDa.

In certain embodiments z of formula (I-ba), (I-bb), (I-bb′), (I-bc) or (I-bc′) is an integer ranging from and including about 11 to about 230. In certain embodiments z of formula (I-ba), (I-bb), (I-bb′), (I-bc) or (I-bc′) is an integer ranging from and including about 11 to about 225. In certain embodiments z of formula (I-ba), (I-bb), (I-bb′), (I-bc) or (I-bc′) is about 11. In certain embodiments z of formula (I-ba), (I-bb), (I-bb′), (I-bc) or (I-bc′) is about 12. In certain embodiments z of formula(I-ba), (I-bb), (I-bb′), (I-bc) or (I-bc′) is about 22. In certain embodiments z of formula (I-ba), (I-bb), (I-bb′), (I-bc) or (I-bc′) is about 23. In certain embodiments z of formula (I-ba), (I-bb), (I-bb′), (I-bc) or (I-bc′) is about 34. In certain embodiments z of formula (I-ba), (I-bb), (I-bb′), (I-bc) or (I-bc′) is about 45. In certain embodiments z of formula (I-ba), (I-bb) or (-I-bc) is about 57. In certain embodiments z of formula (I-ba), (I-bb), (I-bb′), (I-bc) or (I-bc′) is about 68. In certain embodiments z of formula (I-ba), (I-bb), (I-bb′), (I-bc) or (I-bc′) is about 79. In certain embodiments z of formula (I-ba), (I-bb), (I-bb′), (I-bc) or (I-bc′) is about 90. In certain embodiments z of formula (I-ba), (I-bb), (I-bb′), (I-bc) or (I-bc′) is about 102. In certain embodiments z of formula (I-ba), (I-bb), (I-bb′), (I-bc) or (I-bc′) is about 113. In certain embodiments z of formula (I-bb) or (I-bc) is about 135. In certain embodiments z of formula (I-ba), (I-bb), (I-bb′), (I-bc) or (I-bc′) is about 160. In certain embodiments z of formula (I-bb) or (I-bc) is about 180. In certain embodiments z of formula (I-ba), (I-bb), (I-bb′), (I-bc) or (I-bc′) is about 205. In certain embodiments z of formula (I-ba), (I-bb), (I-bb′), (I-bc) or (I-bc′) is about 225. In certain embodiments z of formula (I-ba), (I-bb), (I-bb′), (I-bc) or (I-bc′) is about 230.

In certain embodiments the compound comprises one branching point B, such as a branching point selected from the group consisting of

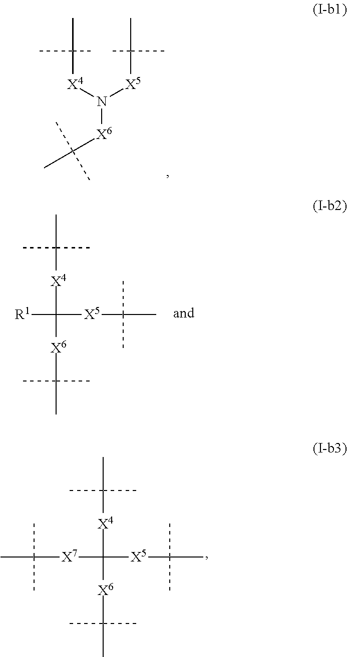

wherein
dashed lines indicate attachment to -A- of formula (Ib);
—R¹ is used as defined for the term “substituent”; and
—X⁴—, —X⁵—, —X⁶— and —X⁷— are independently used as defined for —X¹— and —X²— of formula (I-ai).

In certain embodiments —X⁴—, —X⁵— and —X⁶- of formula (I-b1) and (I-b2) are identical. In certain embodiments —X⁴—, —X⁵— and —X⁶- of formula (I-b1) and (I-b2) are identical and are selected from the group consisting of —CH₂—, —CH₂CH₂- and —CH₂CH₂CH₂—. In certain embodiments the compound comprises one branching point of formula (I-b3), wherein —X⁴—, —X⁵—, —X⁶— and —X⁷— are identical. In certain embodiments the compound comprises one branching point of formula (I-b3), wherein —X⁴—, —X⁵—, —X⁶— and —X⁷— are selected from the group consisting of —CH₂—, —CH₂CH₂- and —CH₂CH₂CH₂—. In certain embodiments the compound comprises one branching point of formula (I-b3), wherein —X⁴—, —X⁵—, —X⁶— and —X⁷— are —CH₂-.

In certain embodiments the compound comprises one branching point of formula (I-b1). In certain embodiments the compound comprises one branching point of formula (I-b2). In certain embodiments the compound comprises one branching point of formula (I-b3).

In certain embodiments the compound is of formula (I-b4)

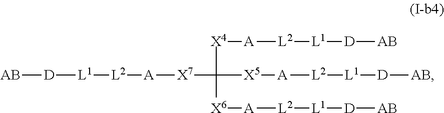

wherein
each -A- is a polymeric moiety;
each -L²- is independently a spacer moiety or is absent;
each -L¹- is independently a linker moiety that is covalently and reversibly conjugated to -D-;
each -D- is independently a drug moiety;
each -AB is independently an albumin-binding moiety;
—X⁴—, —X⁵—, —X⁶— and —X⁷— are independently of each other selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR³)(R^{y3a})—, —N(R^{y3})C(O)N(R^{y3a})—, and —OC(O)N(R^{y3})—;
—R^{y1} and —R^{y1a} are independently of each other selected from the group consisting of —H, -T, C₁₋₅₀ alkyl, C₂₋₅₀ alkenyl, and C₂₋₅₀ alkynyl; wherein -T, C₁₋₅₀ alkyl, C₂₋₅₀ alkenyl, and C₂₋₅₀ alkynyl are optionally substituted with one or more —R^{y2}, which are the same or different, and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR⁴)(R^{y4a})—, —N(R^{y4})C(O)N(R^{y4a})—, and —OC(O)N(R^{y4})—;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;
each —R^{y2} is independently selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and
each —R^{y3}, —R^{y3a}, —R^{y4}, —R^{y4a}, —R^{y5}, —R^{y1a} and —R^{y1b} is independently selected from the group consisting of —H, and C₁₋₆ alkyl, wherein C₁₋₆ alkyl is optionally substituted with one or more halogen, which are the same or different.

In certain embodiments —X⁴—, —X⁵—, —X⁶— and —X⁷— of formula (I-b4) are independently of each other selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₂₀ alkyl, C₂₋₂₀ alkenyl, and C₂₋₂₀ alkynyl are optionally substituted with one or more —R^{y2}, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR³)(R^{y3a})—, —N(R^{y3})C(O)N(R^{y3a})—, and —OC(O)N(R^{y3})—;
—R^{y1} and —R^{y1a} are independently of each other selected from the group consisting of —H, -T, C₁₋₁₀ alkyl, C₂₋₁₀ alkenyl, and C₂₋₁₀ alkynyl; wherein -T, C₁₋₁₀ alkyl, C₂₋₁₀ alkenyl, and C₂₋₁₀ alkynyl are optionally substituted with one or more —R^{y2}, which are the same or different, and wherein C₁₋₁₀ alkyl, C₂₋₁₀ alkenyl, and C₂₋₁₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR⁴)(R^{y4a})—, —N(R^{y4})C(O)N(R^{y4a})—, and —OC(O)N(R^{y4})—;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;
—R^{y2} is selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and
each —R^{y3}, —R^{y3a}, —R^{y4}, —R^{y4a}, —R^{y5}, —R^{y5a} and —R^{y5b} is independently of each other selected from the group consisting of —H, and C₁₋₆ alkyl; wherein C₁₋₆ alkyl is optionally substituted with one or more halogen, which are the same or different.

In certain embodiments —X⁴—, —X⁵—, —X⁶— and —X⁷— of formula (I-b4) are independently of each other selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR³)(R^{y3a})—, —N(R^{y3})C(O)N(R^{y3a})—, and —OC(O)N(R^{y3})—;
—R^{y1} and —R^{y1a} are independently selected from the group consisting of —H, -T, C₁₋₁₀ alkyl, C₂₋₁₀ alkenyl, and C₂₋₁₀ alkynyl;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl;
each —R^{y2} is independently selected from the group consisting of halogen, and C₁₋₆ alkyl; and
each —R^{y3}, —R^{y3a}, —R^{y4}, —R^{y4a}, —R^{y5}, —R^{y5a} and —R^{y5b} is independently of each other selected from the group consisting of —H, and C₁₋₆ alkyl; wherein C₁₋₆ alkyl is optionally substituted with one or more halogen, which are the same or different.

In certain embodiments —X⁴—, —X⁵—, —X⁶— and —X⁷— of formula (I-b3) and (I-b4) are identical. In certain embodiments —X⁴—, —X⁵—, —X⁶— and —X⁷— of formula (I-b3) and (I-b4) are identical and are selected from the group consisting of —CH₂—, —CH₂CH₂— and —CH₂CH₂CH₂—.

In certain embodiments the moiety

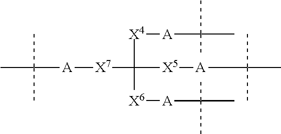

is of formula (I-b5)

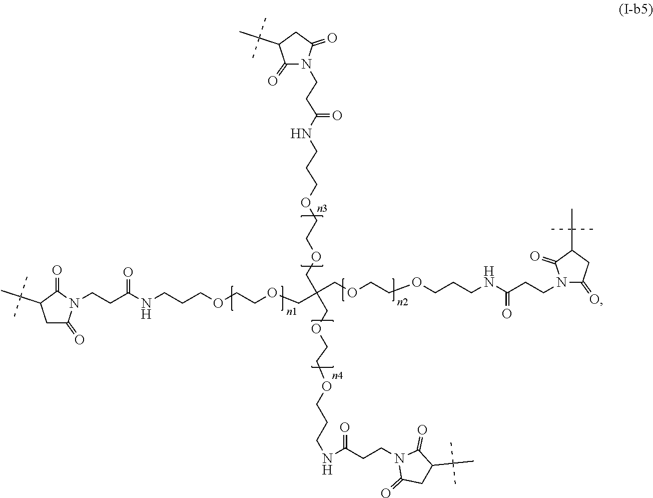

wherein
dashed lines indicate attachment to -L²-; and
n1, n2, n3 and n4 are independently an inter ranging from and including about 10 to about 300, in certain embodiments from and including about 10 to about 230.

In certain embodiments the moiety

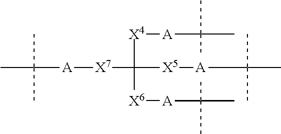

is of formula (I-b6)

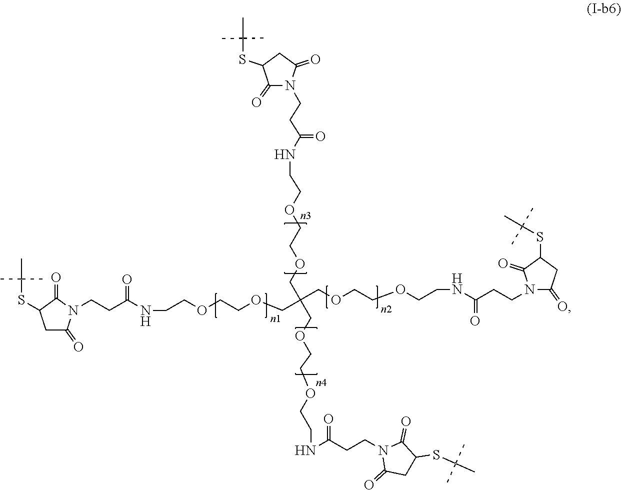

wherein
dashed lines indicate attachment to -L²-; and
n1, n2, n3 and n4 are independently an inter ranging from and including about 10 to about 230.

In certain embodiments n1, n2, n3 and n4 of formula (I-b5) or (I-b6) are the same integer. In certain embodiments n1, n2, n3 and n4 of formula (I-b5) or (I-b6) are about 11. In certain embodiments n1, n2, n3 and n4 of formula (I-b5) or a (I-b6) are about 12. In certain embodiments n1, n2, n3 and n4 of formula (I-b5) or (I-b6) are about 22. In certain embodiments n1, n2, n3 and n4 of formula (I-b5) or (I-b6) are about 23. In certain embodiments n1, n2, n3 and n4 of formula (I-b5) or (I-b6) are about 28. In certain embodiments n1, n2, n3 and n4 of formula (I-b5) or (I-b6) are about 29. In certain embodiments n1, n2, n3 and n4 of formula (I-b5) or (I-b6) are about 34. In certain embodiments n1, n2, n3 and n4 of formula (I-b5) or (I-b6) are about 45. In certain embodiments n1, n2, n3 and n4 of formula (I-b5) or (I-b6) are about 56. In certain embodiments n1, n2, n3 and n4 of formula (I-b5) or (I-b6) are about 68. In certain embodiments n1, n2, n3 and n4 of formula (I-b5) or (I-b6) are about 80. In certain embodiments n1, n2, n3 and n4 of formula (I-b5) or (I-b61 are about 90. In certain embodiments n1, n2. n3 and n4 of formula (I-b5) or (I-b6) are about 100. In certain embodiments n1, n2, n3 and n4 of formula (I-b5) or (I-b6) are about 115. In certain embodiments n1, n2, n3 and n4 of formula (I-b5) or (I-b6) are about 135. In certain embodiments n1, n2, n3 and n4 of formula (I-b5) or (I-b6) are about 160. In certain embodiments n1, n2, n3 and n4 of formula (I-b5) or (I-b6) are about 180.

In certain embodiments n1, n2, n3 and n4 of formula (I-b5) or (I-b6) are about 200. In certain embodiments n1, n2, n3 and n4 of formula (I-b5) or (I-b6) are about 225. In certain embodiments n1, n2, n3 and n4 of formula (I-b5) or (I-b6) are about 230.

In certain embodiments the compound comprises more than one branching point B, such as two or more branching points selected from the group consisting of (I-b1), (I-b2) and (I-b3).

In certain embodiments -D- is a drug moiety selected from the group consisting of small molecule drug moieties, medium size molecule drug moieties, oligonucleotide drug moieties, peptide nucleic acid drug moieties, peptide drug moieties and protein drug moieties.

In certain embodiments -D- is a peptide drug moiety. In certain embodiments -D- is a small molecule drug moiety. In certain embodiments -D- is a medium size drug moiety. In certain embodiments -D- is an oligonucleotide drug moiety. In certain embodiments -D- is a peptide nucleic acid drug moiety. In certain embodiments -D- is a protein drug moiety.

In certain embodiments -D- or -D-AB is a GLP-1 receptor agonist moiety. Accordingly, the compound of the present invention may be a GLP-1 receptor agonist compound.

In certain embodiments -D- or -D-AB is a mono agonist of the GLP-1 receptor, i.e., only activates the GLP-1 receptor.

In certain embodiments -D- or -D-AB is an agonist of the GLP-1 receptor and an agonist of a further receptor, i.e., -D- or -D-AB is a dual GLP-1 receptor agonist. Such further receptor may be selected from the group consisting of the GIP receptor, the GCG receptor, an amylin receptor, a PYY receptor and the GLP-2 receptor.

In certain embodiments -D- or -D-AB is an agonist of the GLP-1 receptor and of the GIP receptor. In certain embodiments -D- or -D-AB is an agonist of the GLP-1 receptor and of the GCG receptor. In certain embodiments -D- or -D-AB is an agonist of the GLP-1 receptor and of an amylin receptor. In certain embodiments -D- or -D-AB is an agonist of the GLP-1 receptor and of a PYY receptor. In certain embodiments -D- or -D-AB is an agonist of the GLP-1 receptor and of the GLP-2 receptor.

In certain embodiments -D or -D-AB is an agonist of the GLP-1 receptor and growth/differentiation factor 15 (GDF15). In certain embodiments -D or -D-AB is an agonist of the GLP-1 receptor and fibroblast growth factor 21 (FGF21).

In certain embodiments -D- or -D-AB is an agonist of the GLP-1 receptor and an agonist of two further receptors, i.e., -D- or -D-AB is a triple GLP-1 receptor agonist. These further receptors are in certain embodiments selected from the group consisting of the GIP receptor (GIPR), the GCG receptor (GCGR), an amylin receptor, a PYY receptor (PYYR) and the GLP-2 receptor (GLP2R).

In certain embodiments -D- or -D-AB is a triple GLP-1 receptor agonist that activates the GLP-1 receptor, the GIP receptor and the GCG receptor. In certain embodiments -D- or -D-AB is a triple GLP-1 receptor agonist that activates the GLP-1 receptor, the GIP receptor and an amylin receptor. In certain embodiments -D or -D-AB is a triple GLP-1 receptor agonist that activates the GLP-1 receptor, the GIP receptor and a PYY receptor. In certain embodiments -D- or -D-AB is a triple GLP-1 receptor agonist that activates the GLP-1 receptor, the GIP receptor and the GLP-2 receptor. In certain embodiments -D- or -D-AB is a triple GLP-1 receptor agonist that activates the GLP-1 receptor, the GCG receptor and an amylin receptor. In certain embodiments -D- or -D-AB is a triple GLP-1 receptor agonist that activates the GLP-1 receptor, the GCG receptor and a PYY receptor. In certain embodiments -D- or -D-AB is a triple GLP-1 receptor agonist that activates the GLP-1 receptor, the GCG receptor and the GLP-2 receptor. In certain embodiments -D- or -D-AB is a triple GLP-1 receptor agonist that activates the GLP-1 receptor, an amylin receptor and a PYY receptor. In certain embodiments -D- or -D-AB is a triple GLP-1 receptor agonist that activates the GLP-1 receptor, the amylin receptor and the GLP-2 receptor. In certain embodiments -D- or -D-AB is a triple GLP-1 receptor agonist that activates the GLP-1 receptor, a PYY receptor and the GLP-2 receptor.

In certain embodiments -D- is a human GLP-1 of SEQ ID NO:1:

HAEGTFTSDVSSYLEGQAAKEFIAWLVKGRG.

In certain embodiments -D- is human GLP-1 analog of SEQ ID NO:1, which peptide sequence may comprise one or more amino acid changes compared to SEQ ID NO:1. Such amino acid changes may be the addition of one or more amino acid residues, the deletion of one or more amino acid residues, the substitution of one or more amino acid residues or may be any combination thereof. Such amino acid change may be at the N-terminus, the C-terminus and/or at an internal site of the GLP-1 of SEQ ID NO:1. In certain embodiments such human GLP-1 analog has a maximum of 3 amino acid changes compared to SEQ ID NO:1, i.e., a maximum of three amino acids are added to, deleted from or substituted compared to the sequence of SEQ ID NO:1.

In certain embodiments -D- has the sequence

(SEQ ID NO: 2)
HX₁EGTFTSDVSSYLEGQAAKEFIAWLVRGRG,

wherein X₁ is 2-aminoisobutyric acid (Aib).

In certain embodiments -D- has the sequence

(SEQ ID NO: 3)
HX₁EGTFTSDVSSYLEGQAAKEFIAWLVRGRG,

wherein X₁ is Aib and the C-terminal glycine, i.e., the glycine at position 31, is amidated as a C-terminal primary amide.

In certain embodiments -D- is exenatide. Exenatide has the sequence

(SEQ ID NO: 4)
HGEGTFTSDLSKQMEEEAVRLFIEWLKNGGPSSGAPPPS.

(SEQ ID NO: 5)
HGEGTFTSDLSKQMEEEAVRLFIEWLKNGGPSSGAPPPS,

(SEQ ID NO: 6)
HGEGTFTSDLSKQMEEEAVRLFIEWLKNGGPSSGAPPSKKKKKK,

In certain embodiments -D has the sequence

(SEQ ID NO: 7)
HGEGTFTSDLSKQMEEEAVRLFIEWLKNGGPSSGAPPSKKKKKK.

In certain embodiments -D- has the sequence

(SEQ ID NO: 8)
HX₁EGTFTSDLSKQX₂EEEAVRLFIEWLKQGGPSSGAPPPC,

wherein X₁ is D-alanine and X₂ is norleucine (Nle).

In certain embodiments -D- has the sequence

(SEQ ID NO: 9)
HX₁EGTFTSDLSKQX₂EEEAVRLFIEWLKQGGPSSGAPPPC,

wherein X₁ is D-alanine, X₂ is Nle and the C-terminal cysteine, i.e., the cysteine at position is amidated as a C-terminal primary amide.

In certain embodiments -D- is PEG-loxenatide. PEG-loxenatide has the sequence

(SEQ ID NO: 10)
HX₁EGTFTSDLSKQX₂EEEAVRLFIEWLKQGGPSSGAPPPC,

wherein X₁ is D-alanine; X₂ is Nle; the cysteine at position 39 is chemically modified through conjugation to the thiol group of the cysteine side-chain with

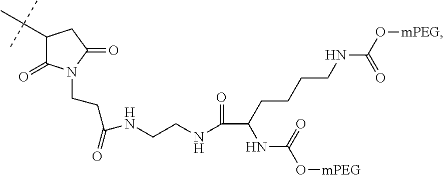

wherein
the dashed line indicates attachment to the thiol group of the cysteine side chain of the cysteine at position 39, and each mPEG is methoxypoly(ethylene glycol) with a molecular weight of approx. 20 kDa.

In certain embodiments -D- has the sequence

(SEQ ID NO: 11)
HX₁EGTFTSDLSKQX₂EEEAVRLFIEWLKQGGPSSGAPPPC,

wherein X₁ is D-alanine; X₂ is Nle; the cysteine at position 39 is chemically modified through conjugation to the thiol group of the cysteine side-chain with

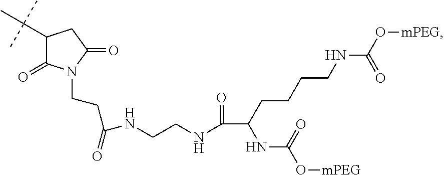

wherein
the dashed line indicates attachment to the thiol group of the cysteine side chain of the cysteine at position 39, each mPEG is methoxypoly(ethylene glycol) with a molecular weight of approx. 20 kDa, and the C-terminal cysteine, i.e., the cysteine at position 39, is amidated as a C-terminal primary amide.

In certain embodiments -D- has the sequence

(SEQ ID NO: 12)
HAEGTFTSDVSSYLEGQAAKEFIAWLVRGRG.

In certain embodiments -D- has the sequence

(SEQ ID NO: 13)
HAEGTFTSDVSSYLEGQAAKEFIAWLVRGRG,

and the C-terminal glycine, i.e., the glycine at position 31, is amidated as a C-terminal primary amide.

In certain embodiments -D- has the sequence

(SEQ ID NO: 14)
HVEGTFTSDVSSYLEEQAAREFIKWLVRGRG.

In certain embodiments -D- has the sequence

(SEQ ID NO: 15)
HVEGTFTSDVSSYLEEQAAREFIKWLVRGRG,

and the C-terminal glycine, i.e., the glycine at position 31, is amidated as a C-terminal primary amide.

In certain embodiments -D- has the sequence

(SEQ ID NO: 16)
HGEGTFTSDVSSYLEGQAAKEFIAWLVRGRG.

In certain embodiments -D- has the sequence

(SEQ ID NO: 17)
HGEGTFTSDVSSYLEGQAAKEFIAWLVRGRG,

and the C-terminal glycine, i.e., the glycine at position 31, is amidated as a C-terminal primary amide.

In certain embodiments -D- has the sequence

(SEQ ID NO: 18)
HX₁EGTFTSDVSSYLEGQAAKEFIAWLVRGRGL,

wherein X₁ is Aib.

In certain embodiments -D- has the sequence

(SEQ ID NO: 19)
HX₁EGTFTSDVSSYLEGQAAKEFIAWLVRGRGL,

wherein X₁ is Aib and the C-terminal leucine, i.e., the leucine at position 32, is amidated as a C-terminal primary amide.

In certain embodiments -D- has the sequence

(SEQ ID NO: 20)
YX₁EGTFTSDYSIX₂LDKIAQKAFVQWLIAGGPSSGAPPPS,

wherein X₁ is Aib, X₂ is Aib and the C-terminal serine, i.e., the serine at position 39, is amidated as a C-terminal primary amide.

In certain embodiments -D- has the sequence

(SEQ ID NO: 21)
YX₁EGTFTSDYSIX₂LDKIAQKAFVQWLIAGGPSSGAPPPS,

wherein X₁ is Aib and X₂ is Aib.

In certain embodiments -D- has the sequence

(SEQ ID NO: 22)
HSQGTFTSDKSEYLDSERARDFVAWLEAGG.

In certain embodiments -D- has the sequence

(SEQ ID NO: 23)
HSQGTFTSDKSEYLDSERARDFVAWLEAGG,

wherein the C-terminal glycine, i.e., the glycine at position 30, is amidated as a C-terminal primary amide.

In certain embodiments -D- has the sequence

(SEQ ID NO: 24)
HX₁QGTFTSDYSKYLDERAAKDFIKWLESA,

wherein X₁ is 1-amino-cyclobutanecarboxylic acid (Ac4c); and the C-terminal alanine, i.e. the alanine at position 29, is amidated as a C-terminal primary amide.

In certain embodiments -D- has the sequence

(SEQ ID NO: 25)
HX₁QGTFTSDYSKYLDERAAKDFIKWLESA,

wherein X₁ is 1-amino-cyclobutanecarboxylic acid (Ac4c).

In certain embodiments -D- has the sequence

(SEQ ID NO: 26)
HX₁QGTFTSDYSKYLDEKAAKEFIQWLLQT,

wherein X₁ is Aib and the glutamic acid at position 16 and the lysine at position 20 are connected via a lactam bridge.

In certain embodiments -D- has the sequence

(SEQ ID NO: 27)
HX₁QGTFTSDYSKYLDEKAAKEFIQWLLQT,

wherein X₁ is Aib, the glutamic acid at position 16 and the lysine at position 20 are connected via a lactam bridge and the C-terminal threonine, i.e., the threonine at position 29, is amidated as a C-terminal primary amide.

In certain embodiments -D- has the sequence

(SEQ ID NO: 28)
HX₁QGTFTSDYSKYLDEKKAKEFVEWLLEGGPSSG,

wherein X₁ is Aib and the C-terminal glycine, i.e., the glycine at position 34, is amidated as a C-terminal primary amide.

In certain embodiments -D- has the sequence

(SEQ ID NO: 29)
HX₁QGTFTSDYSKYLDEKKAKEFVEWLLEGGPSSG,

wherein X₁ is Aib.

In certain embodiments -D- has the sequence

(SEQ ID NO: 30)
HX₁EGSFTSELATILDKQAARDFIAWLIQHKITD,

wherein X₁ is Aib.

In certain embodiments -D- has the sequence

(SEQ ID NO: 31)
HX₁EGSFTSELATILDKQAARDFIAWLIQHKITD,

wherein X₁ is Aib and the C-terminal aspartic acid, i.e., the aspartic acid at position 33, is amidated as a C-terminal primary amide.

In certain embodiments -D- has the sequence

(SEQ ID NO: 32)
YX₁QGTFTSDYSIX₂LDKKAQX₃AFIEYLLEGGPSSGAPPPS,

wherein X₁ is Aib, X₂ is α-methyl-leucine (αMeL), X₃ is Aib; and the C-terminal serine, i.e. the serine at position 39, is amidated as a C-terminal primary amide.

In certain embodiments -D- has the sequence

(SEQ ID NO: 33)
YX₁QGTFTSDYSIX₂LDKKAQX₃AFIEYLLEGGPSSGAPPPS,

wherein X₁ is Aib, X₂ is α-methyl-leucine (αMeL) and X₃ is Aib.

In certain embodiments -D- has the sequence

(SEQ ID NO: 54)
HX₁EGTFTSDVSSYLEEEAAKEFIAWLVRGGPSSGAPPPSK,

wherein X₁ is Aib.

In certain embodiments -D- has the sequence

(SEQ ID NO: 55)
HX₁EGTFTSDVSSYLEEQAAKEFIAWLVRGGG,

wherein X₁ is Aib.

In certain embodiments -D- has the sequence

(SEQ ID NO: 56)
HX₁EGTFTSDVSSYLEEQAAKEFIAWLVRGGGGAQPGAQPGAQPGAQP
GAQPGAQPGAQPGAQPGAQPGQKP,

wherein X₁ is Aib.

In certain embodiments -D- is a drug selected from the group consisting of insulins; amylin and amylin/calcitonin; PYY; GIP; MSH; C5a binders; GDF15; PCSK9 I; immune stimulants; urocortin2; MIC-1; IL-1R antagonists; leptin; gastrin; glucagon; exendin-4; GLP-1; GLP-2; and GIP.

In certain embodiments -D- is a drug moiety selected from the group consisting of insulin; insulin analogues; amylin; dual amylin/calcitonin agonists; PYY; GIP; MSH; C5 inhibitors or modulators; GDF15; PCSK9 inhibitors; immune stimulants; urocortin II; MIC-1; IL-1R antagonists; leptin; gastrin; glucagon; oxyntomodulin; neurokinin A; tachykinin/neurokinin receptor 2 (NK2R) agonists; neurokinin receptor (NKR) agonists and GLP-2.

In certain embodiments -D- is an insulin, such as insulin detemir, insulin degludec and insulin. In certain embodiments -D- is amylin and amylin/calcitonin, such as for example cagrilintide. In certain embodiments -D- is PYY, such as NNC0165-1875. In certain embodiments -D- is GIP. In certain embodiments -D- is MSH. In certain embodiments -D- is a C5a binder, such as zilucoplan. In certain embodiments -D- is GDF15, such as NN LA-GDF15. In certain embodiments -D- is PCSK9 i. In certain embodiments -D- is an immune stimulant, such as romurtide or mifamurtide. In certain embodiments -D- is muramyl dipeptide. In certain embodiments -D- is urocortin2. In certain embodiments -D- is MIC-1. In certain embodiments -D- is an IL-1R antagonist. In certain embodiments -D- is leptin. In certain embodiments -D- is gastrin.

In certain embodiments -D- is selected from the list consisting of growth hormones, such as human growth hormone; FGF21; EGF(a); and coagulations factors.

In certain embodiments -D- is a growth hormone, such as a human growth hormone, such as somapacitan. In certain embodiments -D- is FGF21, such as NNC0194 0499. In certain embodiments -D- is EGF(a). In certain embodiments -D- is a coagulation factor.

In certain embodiments -D- is selected from cytotoxic small molecule drugs; chemotherapy small molecule drugs; and immune activating small molecule drugs.

In certain embodiments -D- is a cytotoxic small molecule drug. In certain embodiments -D- is a chemotherapy small molecule drug. In certain embodiments -D- is and immune activating small molecule drug, such as telratolimod.

In certain embodiments -D- is paclitaxel. In certain embodiments -D- is doxorubicin. In certain embodiments -D- is 5-FU.

In certain embodiments -D- is a PTH moiety.

In certain embodiments -AB is a fatty acid-based albumin-binding moiety.

A moiety -AB binds to albumin, such as human albumin, under physiological conditions (aqueous buffer pH 7.4, 37° C.).

A moiety -D- may be conjugated to one moiety -AB. A moiety -D- may be conjugated to more than one moiety -AB. In certain embodiments the linkage between -D- and a moiety -AB is a stable linkage. In case a moiety -D- comprises two moieties -AB, the linkage between -D- and a first moiety -AB may be reversible and the linkage between -D- and a second moiety -AB may be stable. Alternatively, in case a moiety -D- comprises two moieties -AB, the linkage between -D- and both moieties -AB may be stable.

In certain embodiments -AB of formula (A):

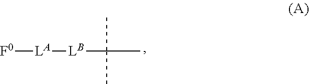

wherein
the dashed line indicates attachment to -D-;
—F⁰ is of formula (a-1)

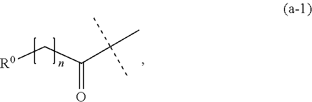

wherein
the dashed line indicates attachment to -L^{A}-;
—R⁰ is selected from the group consisting of —CR¹R^{1a}R^{1b}, —COOR¹,

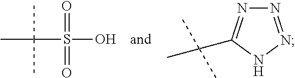

—R¹, —R^{1a} and —R^{1b} are selected from the group consisting of —H, methyl, ethyl, propyl and isopropyl;
n is an integer ranging from and including 8, 9, 10, 11, 12, 13, 14, 15, 16, 17, 18, 19, 20, 21 and 22;
-L^{A}- is absent or is of formula (a-2)

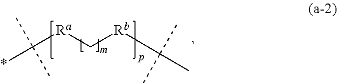

wherein
the unmarked dashed line indicates attachment to -L^{B}-;
the dashed line marked with the asterisk indicates attachment to —F⁰;
—R^{a}— is selected from the group consisting of

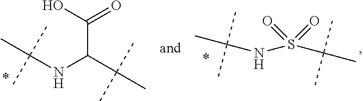

wherein
the dashed line marked with the asterisk indicates attachment to —F⁰;
the unmarked dashed line indicates attachment to the remainder of -L^{A}-;
—R^{b}— is

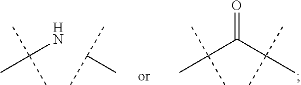

m is an integer selected from the group consisting of 1, 2, 3, 4, 5, 6, 7, 8, 9 and
p is an integer selected from the group consisting of 1, 2, 3, 4, 5, 6, 7, 8, 9 and 10;
-L^{B}- is absent or is of formula (a-3)

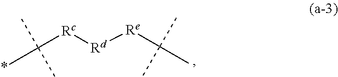

wherein
the unmarked dashed line indicates attachment to -D-;
the dashed line marked with the asterisk indicates attachment to -L^{A};
—R^{c}— is

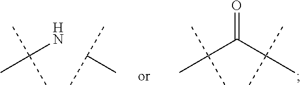

—R^{d}— is selected from the group consisting of C₁₋₅₀ alkyl, C₂₋₅₀ alkenyl and C₂₋₅₀ alkynyl, wherein C₁₋₅₀ alkyl, C₂₋₅₀ alkenyl, and C₂₋₅₀ alkynyl may be substituted with one or more —R¹, which may be the same or different, and which C₁₋₅₀ alkyl, C₂₋₅₀ alkenyl or C₂₋₅₀ alkynyl may be interrupted by one or more groups selected from the group consisting of -T-, —C(O)O—, —O—, —C(O)—, —C(O)N(R²)—, —S(O)₂N(R²)—, —S(O)N(R²)—, —S(O)₂—, —S(O)—, —N(R²)S(O)₂N(R^{2a})—, —S—, —N(R²)—, —OC(OR²)(R^{2a})—, —N(R²)C(O)N(R^{2a})—, and —OC(O)N(R²)—;
   each -T- is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each -T- may independently be substituted with one or more —R¹, which may be the same or different;
   each —R¹ is independently selected from the group consisting of halogen, —CN, oxo (═O), —COOR³, —OR³, —C(O)R³, —C(O)N(R³R^{3a}), —S(O)₂N(R³R^{3a}), —S(O)N (R³R^{3a}), —S(O)₂R³, —S(O)R³, —N(R³)S(O)₂N(R^{3a}R^{3b}), —SR³,—N(R³R^{3a}), —NO₂, —OC(O)R³, —N(R³)C(O)R^{3a}, —N(R³)S(O)₂R^{3a}, —N(R³)S(O)R^{3a}, —N(R³)C(O)OR^{3a}, —N(R³)C(O)N(R^{3a}R^{3b}), —OC(O)N(R³R^{3a}), and C₁₋₆ alkyl; wherein C₁₋₆ alkyl is optionally substituted with one or more halogen, which are the same or different;
   each —R², —R^{2a}, —R³, —R^{3a} and —R^{3b} is independently selected from the group consisting of —H, and C₁₋₆ alkyl, wherein C₁₋₆ alkyl may be substituted with one or more halogen, which may be the same or different; and
—R^{e}— is selected from the group consisting of —CH₂—,

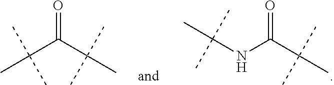

If —R^{b}— is

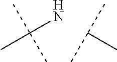

and -L^{B}- is not absent, then —R^{c}— is

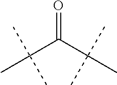

### and if —R^{b}— is

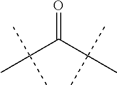

and -L^{B}- is not absent, then —R^{c}— is

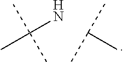

In certain embodiments -L^{A}- is of formula (a-2), i.e., is not absent. In certain embodiments -L^{A}- is absent.

In certain embodiments —R^{a}— is

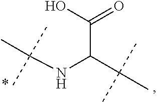

wherein the dashed line marked with the asterisk indicates attachment to —F⁰ and the unmarked dashed line indicates attachment to the remainder of -L^{A}-.

In certain embodiments —R^{a}— is

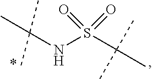

wherein the dashed line marked with the asterisk indicates attachment to —F⁰ and the unmarked dashed line indicates attachment to the remainder of -L^{A}-.

In certain embodiments —R^{b}— is

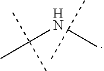

In certain embodiments —R^{b}— is

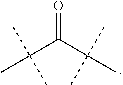

In certain embodiments m of formula (a-2) is 1. In certain embodiments m of formula (a-2) is 2. In certain embodiments m of formula (a-2) is 3. In certain embodiments m of formula (a-2) is 4. In certain embodiments m of formula (a-2) is 5. In certain embodiments m of formula (a-2) is 6. In certain embodiments m of formula (a-2) is 7. In certain embodiments m of formula (a-2) is 8. In certain embodiments m of formula (a-2) is 9. In certain embodiments m of formula (a-2) is 10.

In certain embodiments p of formula (a-2) is 1. In certain embodiments p of formula (a-2) is 2. In certain embodiments p of formula (a-2) is 3. In certain embodiments p of formula (a-2) is 4. In certain embodiments p of formula (a-2) is 5. In certain embodiments p of formula (a-2) is 6. In certain embodiments p of formula (a-2) is 7. In certain embodiments p of formula (a-2) is 8. In certain embodiments p of formula (a-2) is 9. In certain embodiments p of formula (a-2) is 10.

In certain embodiments -L^{B}- is of formula (a-3). In certain embodiments -L^{B}- is absent. If -L^{B}- is absent, the unmarked dashed line in formula (a-2) indicates attachment to -D-.

In certain embodiments —R^{c}— is

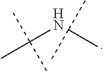

In certain embodiments —R^{c}— is

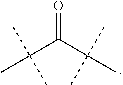

In certain embodiments —R^{c}— is —CH₂—. In certain embodiments —R^{c}— is

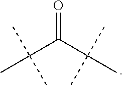

In certain embodiments —R^{c}— is

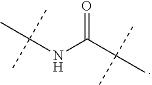

In certain embodiments both -L^{A}- and -L^{B}- are absent. If both -L^{A}- and -L^{B}- are absent, the dashed line in formula (a-1) indicates attachment to -D-.

In certain embodiments —F⁰ is selected from the group consisting of

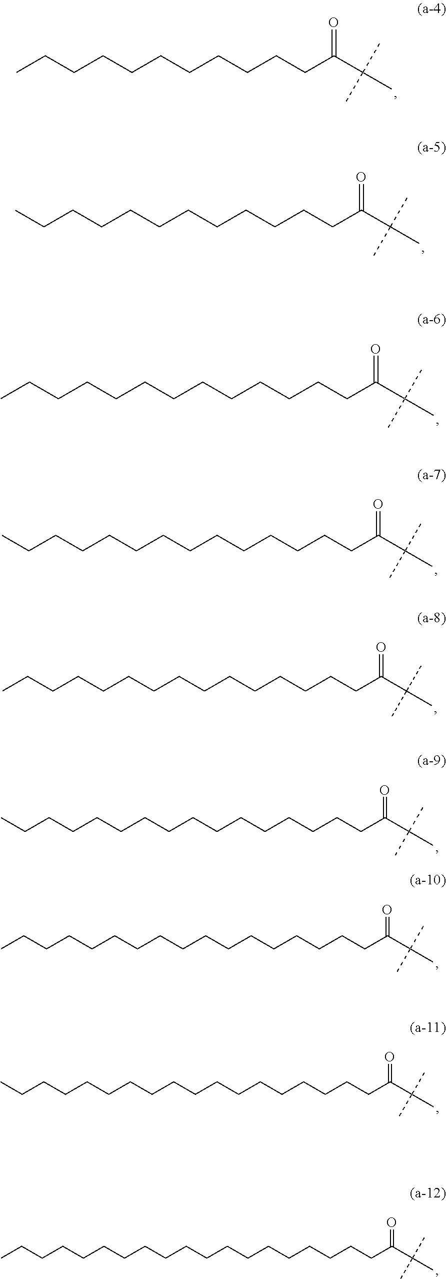

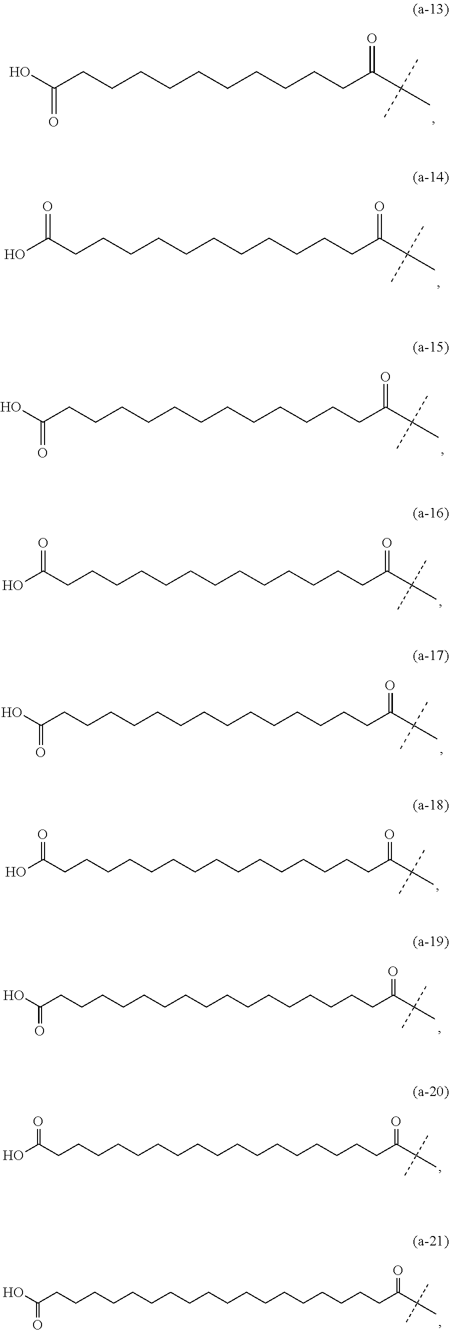

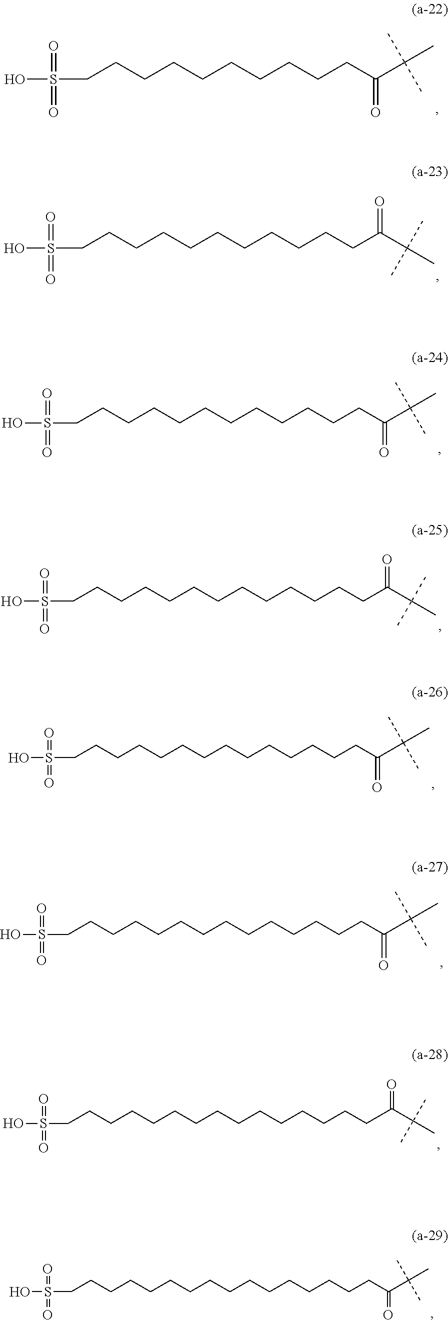

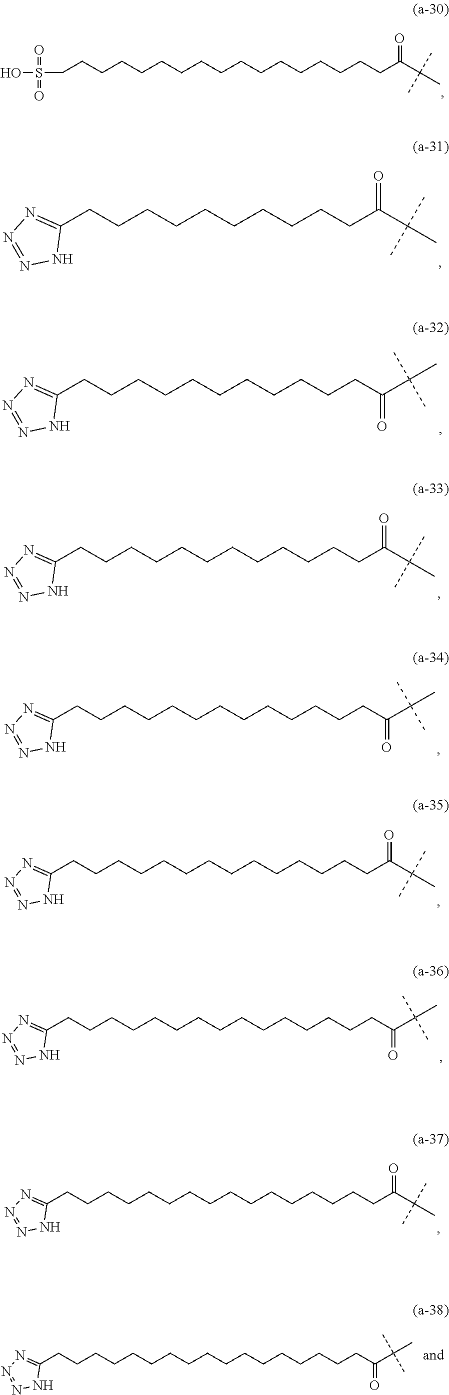

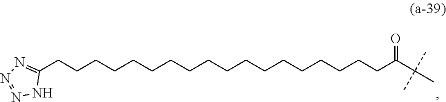

wherein dashed lines indicate attachment to -L^{A}-.

If -L^{A}- is absent, the dashed line in formulas (a-4) to (a-39) indicates attachment to -L^{B} f both -L^{A}- and -L^{B}- are absent, the dashed line in formulas (a-4) to (a-39) indicates attachment to -D-.

In certain embodiments —F⁰ is of formula (a-4). In certain embodiments —F⁰ is of formula (a-5). In certain embodiments —F⁰ is of formula (a-6). In certain embodiments —F⁰ is of formula (a-7). In certain embodiments —F⁰ is of formula (a-8). In certain embodiments —F⁰ is of formula (a-9). In certain embodiments —F⁰ is of formula (a-10). In certain embodiments —F⁰ is of formula (a-I 1). In certain embodiments —F⁰ is of formula (a-12). In certain embodiments —F⁰ is of formula (a-13). In certain embodiments —F⁰ is of formula (a-14). In certain embodiments —F⁰ is of formula (a-15). In certain embodiments —F⁰ is of formula (a-16). In certain embodiments —F⁰ is of formula (a-17). In certain embodiments —F⁰ is of formula (a-18). In certain embodiments —F⁰ is of formula (a-19). In certain embodiments —F⁰ is of formula (a-20). In certain embodiments —F⁰ is of formula (a-21). In certain embodiments —F⁰ is of formula (a-22). In certain embodiments —F⁰ is of formula (a-23). In certain embodiments —F⁰ is of formula (a-24). In certain embodiments —F⁰ is of formula (a-25). In certain embodiments —F⁰ is of formula (a-26). In certain embodiments —F⁰ is of formula (a-27). In certain embodiments —F⁰ is of formula (a-28). In certain embodiments —F⁰ is of formula (a-29). In certain embodiments —F⁰ is of formula (a-30). In Certain embodiments —F⁰ is of formula (a-31). In Certain embodiments —F⁰ is of formula (a-32). In certain embodiments —F⁰ is of formula (a-33). In certain embodiments —F⁰ is of formula (a-34). In certain embodiments —F⁰ is of formula (a-35). In certain embodiments —F⁰ is of formula (a-36). In certain embodiments —F⁰ is of formula (a-37). In Certain embodiments —F⁰ is of formula (a-38). In Certain embodiments —F⁰ is of formula (a-39).

In certain embodiments —F⁰ is of formula (a-4) and both -L^{A}- and -L^{B}- are absent. In certain embodiments —F⁰ is of formula (a-5) and both -L^{A}- and -L^{B}- are absent. In certain embodiments —F⁰ is of formula (a-6) and both -L^{A}- and -L^{B}- are absent. In certain embodiments —F⁰ is of formula (a-7) and both -L^{A}- and -L^{B}- are absent. In certain embodiments —F⁰ is of formula (a-8) and both -L^{A}- and -L^{B}- are absent. In certain embodiments —F⁰ is of formula (a-9) and both -L^{A}- and -L^{B}- are absent. In certain embodiments —F⁰ is of formula (a-10) and both -L^{A}- and -L^{B}- are absent. In certain embodiments —F⁰ is of formula (a-11) and both -L^{A}- and -L^{B}- are absent. In certain embodiments —F⁰ is of formula (a-12) and both -L^{A}- and -L^{B}- are absent. In certain embodiments —F⁰ is of formula (a-13) and both -L^{A}- and -L^{B}- are absent. In certain embodiments —F⁰ is of formula (a-14) and both -L^{A}- and -L^{B}- are absent. In certain embodiments —F⁰ is of formula (a-15) and both -L^{A}- and -L^{B}- are absent. In certain embodiments —F⁰ is of formula (a-16) and both -L^{A}- and -L^{B}- are absent. In certain embodiments —F⁰ is of formula (a-17) and both -L^{A}- and -L^{B}- are absent. In certain embodiments —F⁰ is of formula (a-18) and both -L^{A}- and -L^{B}- are absent. In certain embodiments —F⁰ is of formula (a-19) and both -L^{A}- and -L^{B}- are absent. In certain embodiments —F⁰ is of formula (a-20) and both -L^{A}- and -L^{B}- are absent. In certain embodiments —F⁰ is of formula (a-21) and both -L^{A}- and -L^{B}- are absent. In certain embodiments —F⁰ is of formula (a-22) and both -L^{A}- and -L^{B}- are absent. In certain embodiments —F⁰ is of formula (a-23) and both -L^{A}- and -L^{B}- are absent. In certain embodiments —F⁰ is of formula (a-24) and both -L^{A}- and -L^{B}- are absent. In certain embodiments —F⁰ is of formula (a-25) and both -L^{A}- and -L^{B}- are absent. In certain embodiments —F⁰ is of formula (a-26) and both -L^{A}- and -L^{B}- are absent. In certain embodiments —F⁰ is of formula (a-27) and both -L^{A}- and -L^{B}- are absent. In certain embodiments —F⁰ is of formula (a-28) and both -L^{A}- and -L^{B}- are absent. In certain embodiments —F⁰ is of formula (a-29) and both -L^{A}- and -L^{B}- are absent. In certain embodiments —F⁰ is of formula (a-30) and both -L^{A}- and -L^{B}- are absent. In certain embodiments —F⁰ is of formula (a-31) and both -L^{A}- and -L^{B}- are absent. In certain embodiments —F⁰ is of formula (a-32) and both -L^{A}- and -L^{B}- are absent. In certain embodiments —F⁰ is of formula (a-33) and both -L^{A}- and -L^{B}- are absent. In certain embodiments —F⁰ is of formula (a-34) and both -L^{A}- and -L^{B}- are absent. In certain embodiments —F⁰ is of formula (a-35) and both -L^{A}- and -L^{B}- are absent. In certain embodiments —F⁰ is of formula (a-36) and both -L^{A}- and -L^{B}- are absent. In certain embodiments —F⁰ is of formula (a-37) and both -L^{A}- and -L^{B}- are absent. In certain embodiments —F⁰ is of formula (a-38) and both -L^{A}- and -L- are absent. In certain embodiments —F⁰ is of formula (a-39) and both -L^{A}- and -L^{B}- are bet

In certain embodiments -L^{A}- is selected from the group consisting of

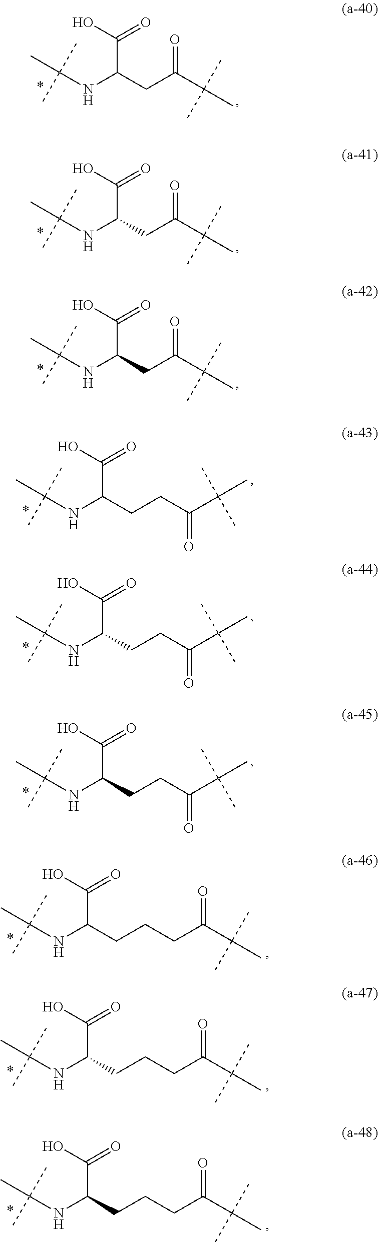

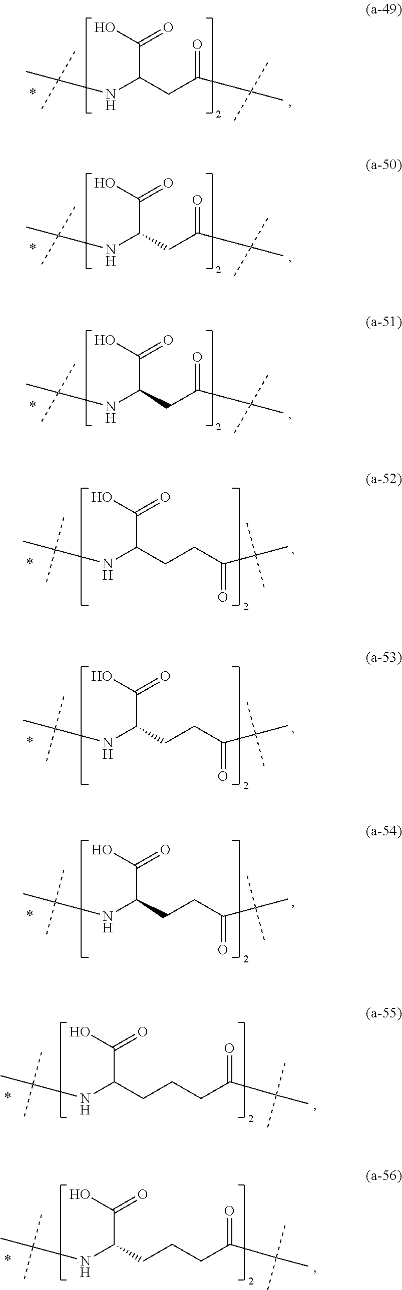

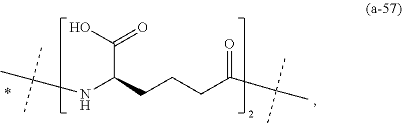

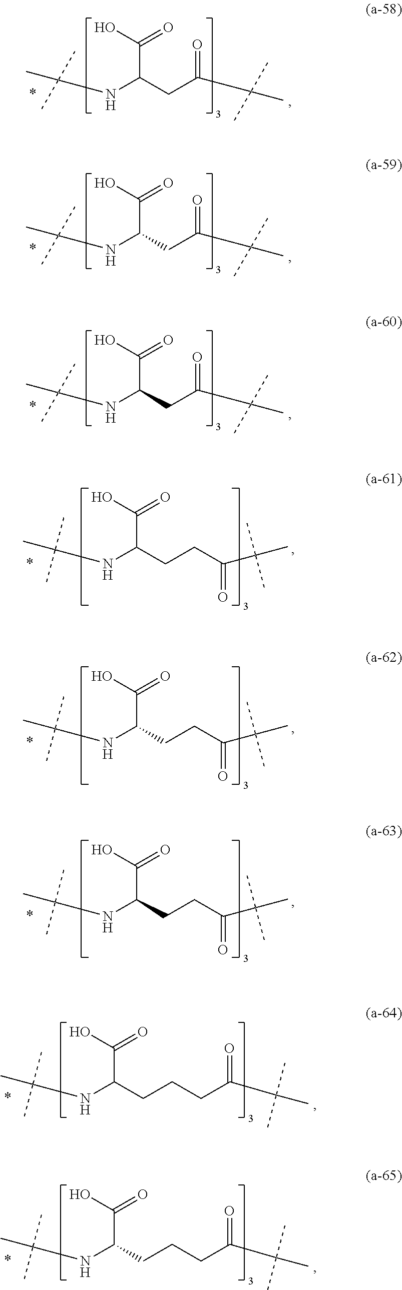

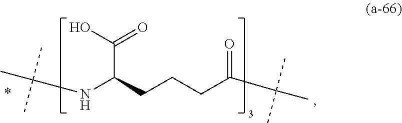

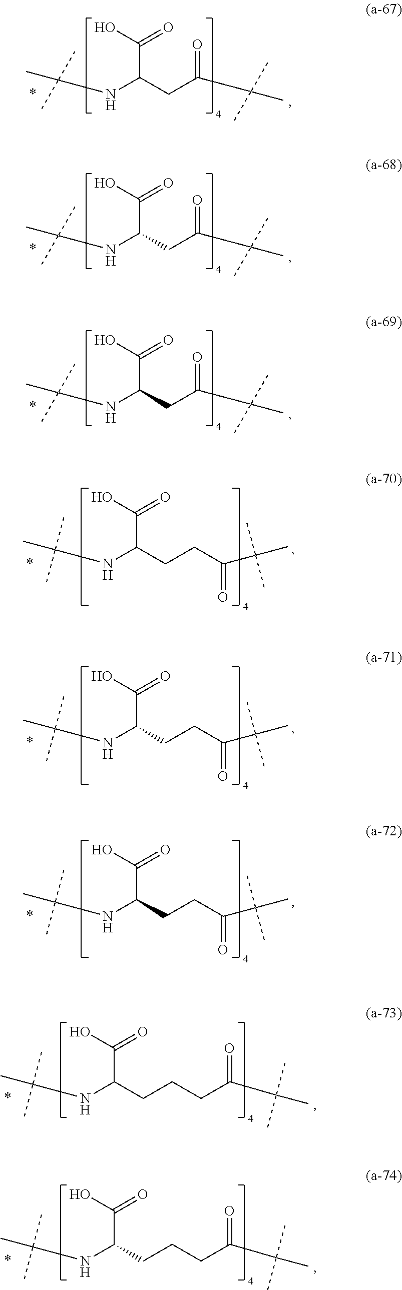

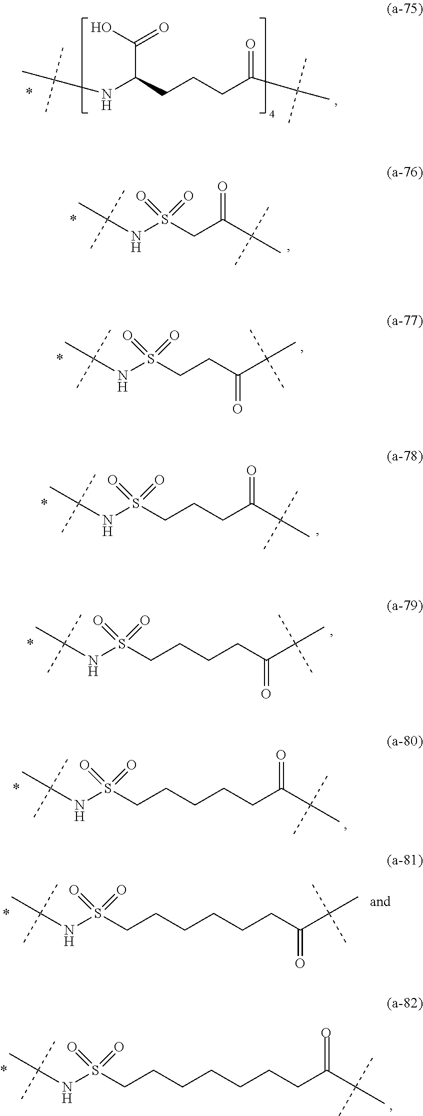

wherein a dashed line marked with an asterisk indicates attachment to —F⁰— and an unmarked dashed line indicates attachment to -L^{B}- If -L^{B}- is absent an unmarked dashed indicates attachment to -D-.

In certain embodiments -L^{A}- is of formula (a-40). In certain embodiments -L^{A}- is of formula (a-41). In certain embodiments -L^{A}- is of formula (a-42). In certain embodiments -L^{A}- is of formula (a-43). In certain embodiments -L^{A}- is of formula (a-44). In certain embodiments -L^{A}- is of formula (a-45). In certain embodiments -L^{A}- is of formula (a-46). In certain embodiments -L^{A}- is of formula (a-47). In certain embodiments -L^{A}- is of formula (a-48). In certain embodiments -L^{A}- is of formula (a-49). In certain embodiments -L^{A}- is of formula (a-50). In certain embodiments -L^{A}- is of formula (a-51). In certain embodiments -L^{A}- is of formula (a-52). In certain embodiments -L^{A}- is of formula (a-53). In certain embodiments -L^{A}- is of formula (a-54). In certain embodiments -L^{A}- is of formula (a-55). In certain embodiments -L^{A}- is of formula (a-56). In certain embodiments -L^{A}- is of formula (a-57). In certain embodiments -L^{A}- is of formula (a-58). In certain embodiments -L^{A}- is of formula (a-59). In certain embodiments -L^{A}- is of formula (a-60). In certain embodiments -L^{A}- is of formula (a-61). In certain embodiments -L^{A}- is of formula (a-62). In certain embodiments -L^{A}- is of formula (a-63). In certain embodiments -L^{A}- is of formula (a-64). In certain embodiments -L^{A}- is of formula (a-65). In certain embodiments -L^{A}- is of formula (a-66). In certain embodiments -L^{A}- is of formula (a-67). In certain embodiments -L^{A}- is of formula (a-68). In certain embodiments -L^{A}- is of formula (a-69). In certain embodiments -L^{A}- is of formula (a-70). In certain embodiments -L^{A}- is of formula (a-71). In certain embodiments -L^{A}- is of formula (a-72). In certain embodiments -L^{A}- is of formula (a-73). In certain embodiments -L^{A}- is of formula (a-74). In certain embodiments -L^{A}- is of formula (a-75). In certain embodiments -L^{A}- is of formula (a-76). In certain embodiments -L^{A}- is of formula (a-77). In certain embodiments -L^{A}- is of formula (a-78). In certain embodiments -L^{A}- is of formula (a-79). In certain embodiments -L^{A}- is of formula (a-80). In certain embodiments -L^{A}- is of formula (a-81). In certain embodiments -L^{A}- is of formula (a-82).

In certain embodiments -L^{B}- is selected from the group consisting of

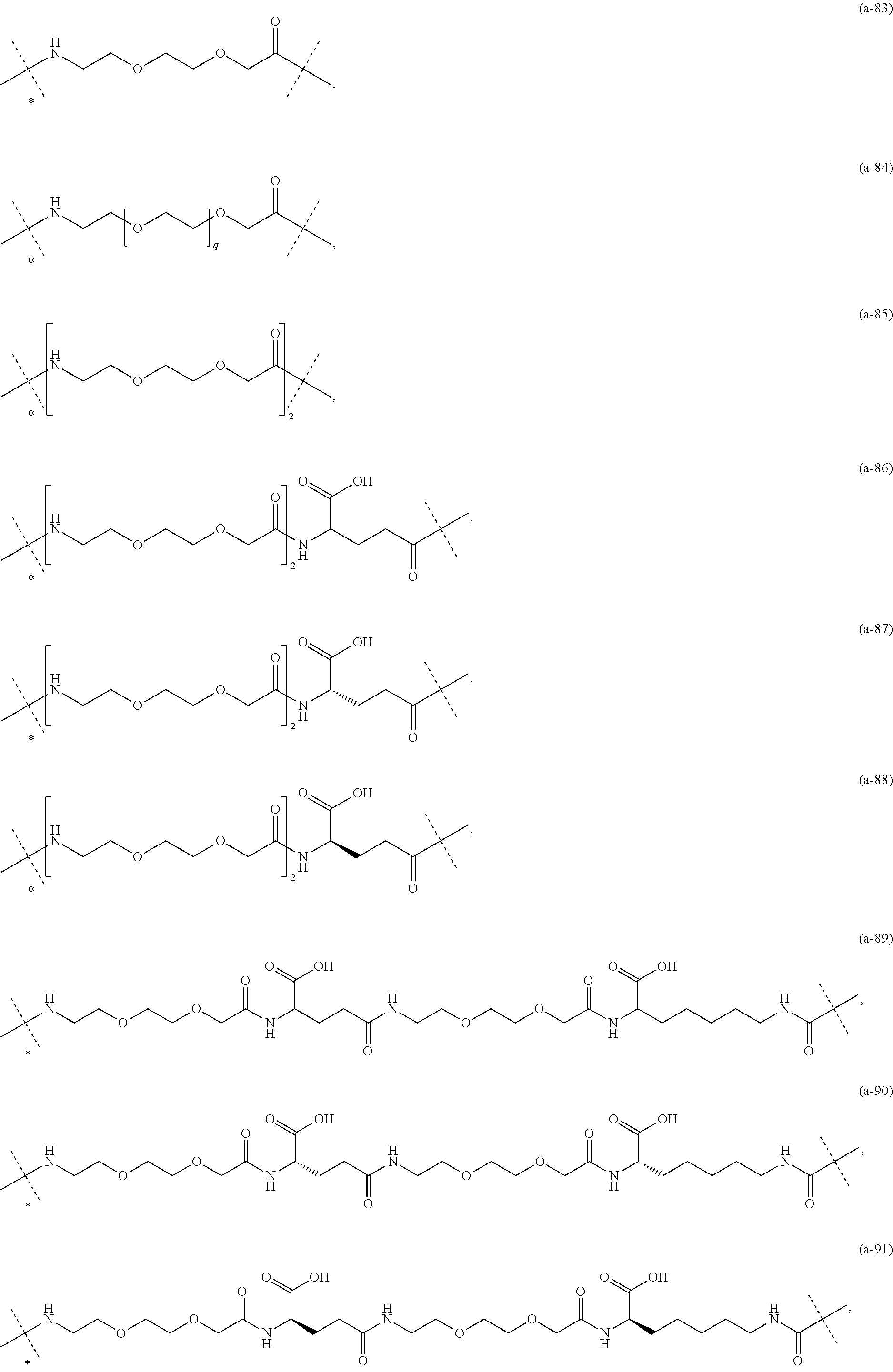

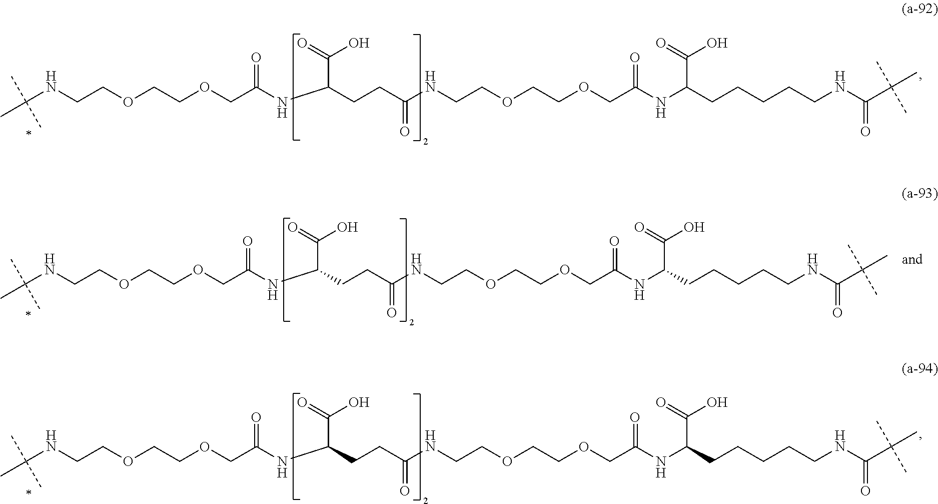

wherein
the dashed line marked with the asterisk indicates attachment to -L^{A}-;
the unmarked dashed line indicates attachment to -D-; and
q is an integer ranging from and including 2 to 50.

If -L^{A}- is absent, the dashed line marked with the asterisk in formulas (a-83) to (a-94) indicates attachment to —F⁰.

In certain embodiments q of formula (a-84) is an integer ranging from and including 3 to 45. In certain embodiments q of formula (a-84) is an integer ranging from and including 4 to 40. In certain embodiments q of formula (a-84) is an integer ranging from and including 5 to 35. In certain embodiments q of formula (a-84) is an integer ranging from and including 6 to 30. In certain embodiments q of formula (a-84) is an integer ranging from and including 7 to 25. In certain embodiments q of formula (a-84) is an integer ranging from and including 10 to 20. In certain embodiments q of formula (a-84) is 23.

In certain embodiments -L^{B}- is of formula (a-83). In certain embodiments -L^{B}- is of formula (a-84). In certain embodiments -L^{B}- is of formula (a-84) with q=23. In certain embodiments -L^{B}- is of formula (a-85). In certain embodiments -L^{B}- is of formula (a-86). In certain embodiments -L^{B}- is of formula (a-87). In certain embodiments -L^{B}- is of formula (a-88). In certain embodiments -L^{B}- is of formula (a-89). In certain embodiments -L^{B}- is of formula (a-90). In certain embodiments -L^{B}- is of formula (a-91). In certain embodiments -L^{B}- is of formula (a-92). In certain embodiments -L^{B}- is of formula (a-93). In certain embodiments -L^{B}- is of formula (a-94).

In certain embodiments -AB is of formula (i)

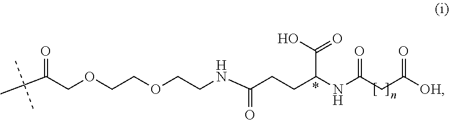

wherein the dashed line indicates attachment to -D-;
n is an integer ranging from and including 14 to 22; and
the stereocenter marked with the asterisk is either in S- or R-configuration.

In certain embodiments -AB is of formula (i) and n is 14. In certain embodiments -AB is of formula (i) and n is 15. In certain embodiments -AB is of formula (i) and n is 16. In certain embodiments -AB is of formula (i) and n is 17. In certain embodiments -AB is of formula (i) and n is 18. In certain embodiments -AB is of formula (i) and n is 19. In certain embodiments -AB is of formula (i) and n is 20. In certain embodiments -AB is of formula (i) and n is 21. In certain embodiments -AB is of formula (i) and n is 22.

In certain embodiments -AB is of formula (i) and n is 14 and the stereocenter marked with the asterisk is in R-configuration. In certain embodiments -AB is of formula (i) and n is 15 and the stereocenter marked with the asterisk is in R-configuration. In certain embodiments -AB is of formula (i) and n is 16 and the stereocenter marked with the asterisk is in R-configuration. In certain embodiments -AB is of formula (i) and n is 17 and the stereocenter marked with the asterisk is in R-configuration. In certain embodiments -AB is of formula (i) and n is 18 and the stereocenter marked with the asterisk is in R-configuration. In certain embodiments -AB is of formula (i) and n is 19 and the stereocenter marked with the asterisk is in R-configuration. In certain embodiments -AB is of formula (i) and n is 20 and the stereocenter marked with the asterisk is in R-configuration. In certain embodiments -AB is of formula (i) and n is 21 and the stereocenter marked with the asterisk is in R-configuration. In certain embodiments -AB is of formula (i) and n is 22 and the stereocenter marked with the asterisk is in R-configuration.

In certain embodiments -AB is of formula (i) and n is 14 and the stereocenter marked with the asterisk is in S-configuration. In certain embodiments -AB is of formula (i) and n is 15 and the stereocenter marked with the asterisk is in S-configuration. In certain embodiments -AB is of formula (i) and n is 16 and the stereocenter marked with the asterisk is in S-configuration. In certain embodiments -AB is of formula (i) and n is 17 and the stereocenter marked with the asterisk is in S-configuration. In certain embodiments -AB is of formula (i) and n is 18 and the stereocenter marked with the asterisk is in S-configuration. In certain embodiments -AB is of formula (i) and n is 19 and the stereocenter marked with the asterisk is in S-configuration. In certain embodiments -AB is of formula (i) and n is 20 and the stereocenter marked with the asterisk is in S-configuration. In certain embodiments -AB is of formula (i) and n is 21 and the stereocenter marked with the asterisk is in S-configuration. In certain embodiments -AB is of formula (i) and n is 22 and the stereocenter marked with the asterisk is in S-configuration.

In certain embodiments -AB is of formula (i-a):

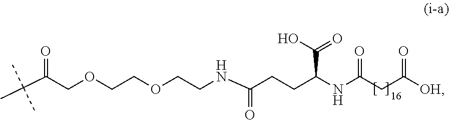

wherein
the dashed line indicates attachment to -D-.

In certain embodiments -AB is of formula (i-b):

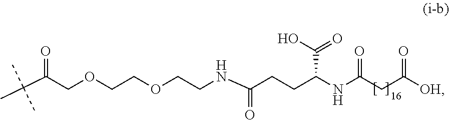

wherein
the dashed line indicates attachment to -D-.

In certain embodiments -AB is of formula (i-c):

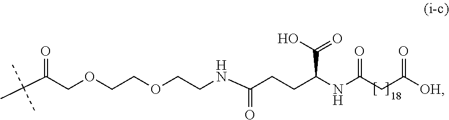

wherein
the dashed line indicates attachment to -D-.

In certain embodiments -AB is of formula (i-d):

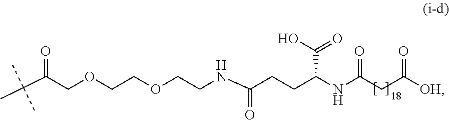

wherein
the dashed line indicates attachment to -D-.

In certain embodiments -AB is of formula (i-e):

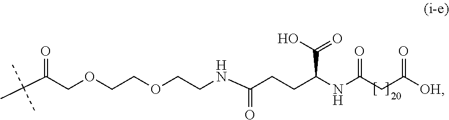

wherein
the dashed line indicates attachment to -D-.

In certain embodiments -AB is of formula (i-f):

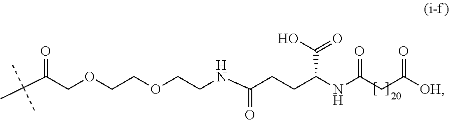

wherein
the dashed line indicates attachment to -D-.

In certain embodiments -AB is of formula (ii)

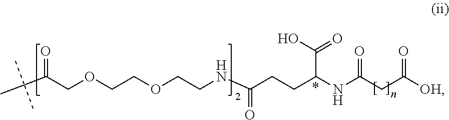

wherein the dashed line indicates attachment to -D-;
n is an integer ranging from 14 to 22; and
the stereocenter marked with the asterisk is either in S- or R-configuration.

In certain embodiments -AB is of formula (ii) and n is 14. In certain embodiments -AB is of formula (ii) and n is 15. In certain embodiments -AB is of formula (ii) and n is 16. In certain embodiments -AB is of formula (ii) and n is 17. In certain embodiments -AB is of formula (ii) and n is 18. In certain embodiments -AB is of formula (ii) and n is 19. In certain embodiments -AB is of formula (ii) and n is 20. In certain embodiments -AB is of formula (ii) and n is 21. In certain embodiments -AB is of formula (ii) and n is 22.

In certain embodiments -AB is of formula (ii) and n is 14 and the stereocenter marked with the asterisk is in R-configuration. In certain embodiments -AB is of formula (ii) and n is 15 and the stereocenter marked with the asterisk is in R-configuration. In certain embodiments -AB is of formula (ii) and n is 16 and the stereocenter marked with the asterisk is in R-configuration. In certain embodiments -AB is of formula (ii) and n is 17 and the stereocenter marked with the asterisk is in R-configuration. In certain embodiments -AB is of formula (ii) and n is 18 and the stereocenter marked with the asterisk is in R-configuration. In certain embodiments -AB is of formula (ii) and n is 19 and the stereocenter marked with the asterisk is in R-configuration. In certain embodiments -AB is of formula (ii) and n is 20 and the stereocenter marked with the asterisk is in R-configuration. In certain embodiments -AB is of formula (ii) and n is 21 and the stereocenter marked with the asterisk is in R-configuration. In certain embodiments -AB is of formula (ii) and n is 22 and the stereocenter marked with the asterisk is in R-configuration.

In certain embodiments -AB is of formula (ii) and n is 14 and the stereocenter marked with the asterisk is in S-configuration. In certain embodiments -AB is of formula (ii) and n is 15 and the stereocenter marked with the asterisk is in S-configuration. In certain embodiments -AB is of formula (ii) and n is 16 and the stereocenter marked with the asterisk is in S-configuration. In certain embodiments -AB is of formula (ii) and n is 17 and the stereocenter marked with the asterisk is in S-configuration. In certain embodiments -AB is of formula (ii) and n is 18 and the stereocenter marked with the asterisk is in S-configuration. In certain embodiments -AB is of formula (ii) and n is 19 and the stereocenter marked with the asterisk is in S-configuration. In certain embodiments -AB is of formula (ii) and n is 20 and the stereocenter marked with the asterisk is in S-configuration. In certain embodiments -AB is of formula (ii) and n is 21 and the stereocenter marked with the asterisk is in S-configuration. In certain embodiments -AB is of formula (ii) and n is 22 and the stereocenter marked with the asterisk is in S-configuration.

In certain embodiments -AB is of formula (ii-a):

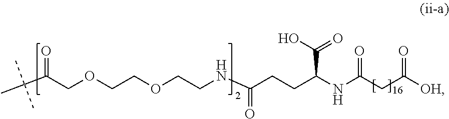

wherein
the dashed line indicates attachment to -D-.

In certain embodiments -AB is of formula (ii-b):

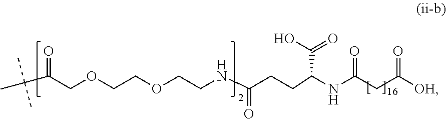

wherein
the dashed line indicates attachment to -D-.

In certain embodiments -AB is of formula (ii-c):

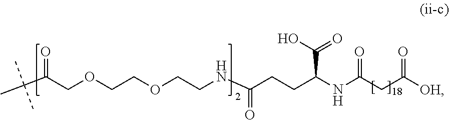

wherein
the dashed line indicates attachment to -D-.

In certain embodiments -AB is of formula (ii-d):

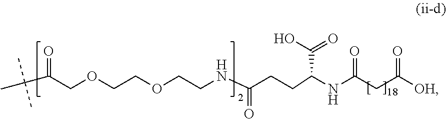

wherein
the dashed line indicates attachment to -D-.

In certain embodiments -AB is of formula (ii-e):

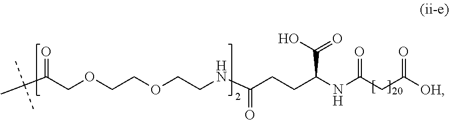

wherein
the dashed line indicates attachment to -D-.

In certain embodiments -AB is of formula (ii-f):

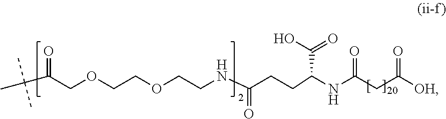

wherein
the dashed line indicates attachment to -D-.

In certain embodiments -AB is of formula (iii)

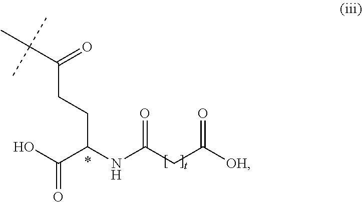

wherein
the dashed line indicates attachment to -D-;
t is an integer ranging from and including 14 to 22; and
the stereocenter marked with the asterisk is either in S- or R-configuration.

In certain embodiments -AB is of formula (iii) and t is 14. In certain embodiments -AB is of formula (iii) and t is 15. In certain embodiments -AB is of formula (iii) and t is 16. In certain embodiments -AB is of formula (iii) and t is 17. In certain embodiments -AB is of formula (iii) and t is 18. In certain embodiments -AB is of formula (iii) and t is 19. In certain embodiments -AB is of formula (iii) and t is 20. In certain embodiments -AB is of formula (iii) and t is 21. In certain embodiments -AB is of formula (iii) and t is 22.

In certain embodiments -AB is of formula (iii) and t is 14 and the stereocenter marked with the asterisk is in R-configuration. In certain embodiments -AB is of formula (iii) and t is 15 and the stereocenter marked with the asterisk is in R-configuration. In certain embodiments -AB is of formula (iii) and t is 16 and the stereocenter marked with the asterisk is in R-configuration. In certain embodiments -AB is of formula (iii) and t is 17 and the stereocenter marked with the asterisk is in R-configuration. In certain embodiments -AB is of formula (iii) and t is 18 and the stereocenter marked with the asterisk is in R-configuration. In certain embodiments -AB is of formula (iii) and t is 19 and the stereocenter marked with the asterisk is in R-configuration.

In certain embodiments -AB is of formula (iii) and t is 20 and the stereocenter marked with the asterisk is in R-configuration. In certain embodiments -AB is of formula (iii) and t is 21 and the stereocenter marked with the asterisk is in R-configuration. In certain embodiments -AB is of formula (iii) and t is 22 and the stereocenter marked with the asterisk is in R-configuration. In certain embodiments -AB is of formula (iii) and t is 14 and the stereocenter marked with the asterisk is in S-configuration. In certain embodiments -AB is of formula (iii) and t is 15 and the stereocenter marked with the asterisk is in S-configuration. In certain embodiments -AB is of formula (iii) and t is 16 and the stereocenter marked with the asterisk is in S-configuration. In certain embodiments -AB is of formula (iii) and t is 17 and the stereocenter marked with the asterisk is in S-configuration. In certain embodiments -AB is of formula (iii) and t is 18 and the stereocenter marked with the asterisk is in S-configuration. In certain embodiments -AB is of formula (iii) and t is 19 and the stereocenter marked with the asterisk is in S-configuration. In certain embodiments -AB is of formula (iii) and t is 20 and the stereocenter marked with the asterisk is in S-configuration. In certain embodiments -AB is of formula (iii) and t is 21 and the stereocenter marked with the asterisk is in S-configuration. In certain embodiments -AB is of formula (iii) and t is 22 and the stereocenter marked with the asterisk is in S-configuration.

In certain embodiments -AB is of formula (iii-a)

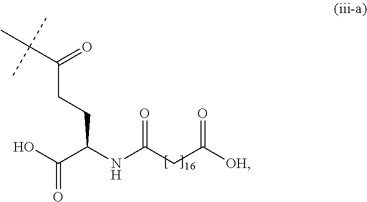

wherein
the dashed line indicates attachment to -D-.

In certain embodiments -AB is of formula (iii-b)

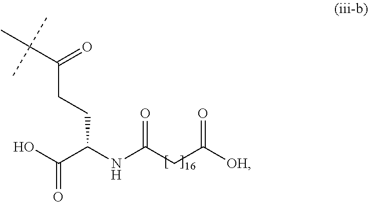

wherein
the dashed line indicates attachment to -D-;

In certain embodiments -AB is of formula (iii-c)

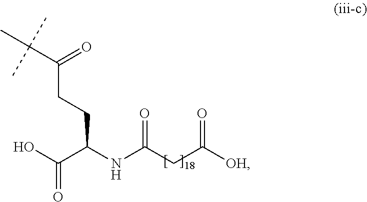

wherein
the dashed line indicates attachment to -D-.

In certain embodiments -AB is of formula (iii-d)

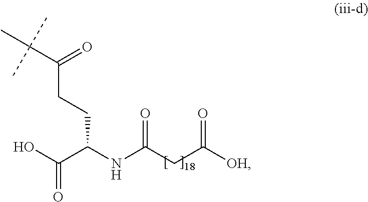

wherein
the dashed line indicates attachment to -D-.

In certain embodiments -AB is of formula (iii-e)

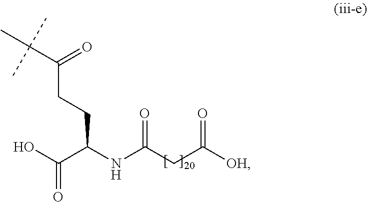

wherein
the dashed line indicates attachment to -D-.

In certain embodiments -AB is of formula (iii-f)

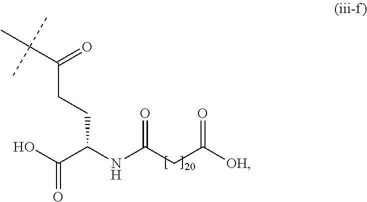

wherein
the dashed line indicates attachment to -D-.

In certain embodiments -AB is of formula (iv)

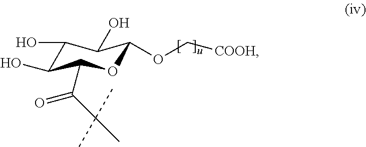

wherein
the dashed line indicates attachment to -D-, and
u is an integer ranging from and including 14 to 22.

In certain embodiments -AB is of formula (iv) and u is 14. In certain embodiments -AB is of formula (iv) and u is 15. In certain embodiments -AB is of formula (iv) and u is 16. In certain embodiments -AB is of formula (iv) and u is 17. In certain embodiments -AB is of formula (iv) and u is 18. In certain embodiments -AB is of formula (iv) and u is 19. In certain embodiments -AB is of formula (iv) and u is 20. In certain embodiments -AB is of formula (iv) and u is 21. In certain embodiments -AB is of formula (iv) and u is 22.

In certain embodiments -AB is of formula (v)

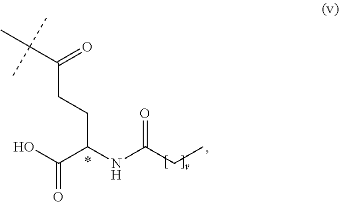

wherein
the dashed line indicates attachment to -D-;
v is an integer ranging from and including 14 to 22; and
the stereocenter marked with the asterisk is either in S- or R-configuration.

In certain embodiments -AB is of formula (v) and v is 14. In certain embodiments -AB is of formula (v) and v is 15. In certain embodiments -AB is of formula (v) and v is 16. In certain embodiments -AB is of formula (v) and v is 17. In certain embodiments -AB is of formula (v) and v is 18. In certain embodiments -AB is of formula (v) and v is 19. In certain embodiments -AB is of formula (v) and v is 20. In certain embodiments -AB is of formula (v) and v is 21. In certain embodiments -AB is of formula (v) and v is 22.

In certain embodiments -AB is of formula (v) and v is 14 and the stereocenter marked with the asterisk is in R-configuration. In certain embodiments -AB is of formula (v) and v is 15 and the stereocenter marked with the asterisk is in R-configuration. In certain embodiments -AB is of formula (v) and v is 16 and the stereocenter marked with the asterisk is in R-configuration.

In certain embodiments -AB is of formula (v) and v is 17 and the stereocenter marked with the asterisk is in R-configuration. In certain embodiments -AB is of formula (v) and v is 18 and the stereocenter marked with the asterisk is in R-configuration. In certain embodiments -AB is of formula (v) and v is 19 and the stereocenter marked with the asterisk is in R-configuration.

In certain embodiments -AB is of formula (v) and v is 20 and the stereocenter marked with the asterisk is in R-configuration. In certain embodiments -AB is of formula (v) and v is 21 and the stereocenter marked with the asterisk is in R-configuration. In certain embodiments -AB is of formula (v) and v is 22 and the stereocenter marked with the asterisk is in R-configuration.

In certain embodiments -AB is of formula (v) and v is 14 and the stereocenter marked with the asterisk is in S-configuration. In certain embodiments -AB is of formula (v) and v is 15 and the stereocenter marked with the asterisk is in S-configuration. In certain embodiments -AB is of formula (v) and v is 16 and the stereocenter marked with the asterisk is in S-configuration. In certain embodiments -AB is of formula (v) and v is 17 and the stereocenter marked with the asterisk is in S-configuration. In certain embodiments -AB is of formula (v) and v is 18 and the stereocenter marked with the asterisk is in S-configuration. In certain embodiments -AB is of formula (v) and v is 19 and the stereocenter marked with the asterisk is in S-configuration. In certain embodiments -AB is of formula (v) and v is 20 and the stereocenter marked with the asterisk is in S-configuration. In certain embodiments -AB is of formula (v) and v is 21 and the stereocenter marked with the asterisk is in S-configuration. In certain embodiments -AB is of formula (v) and v is 22 and the stereocenter marked with the asterisk is in S-configuration.

In certain embodiments -AB is of formula (v-a)

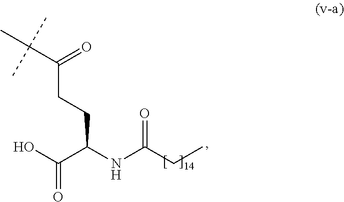

wherein
the dashed line indicates attachment to -D-.

In certain embodiments -AB is of formula (v-b)

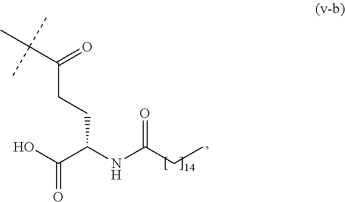

wherein
the dashed line indicates attachment to -D-;

In certain embodiments -AB is of formula (v-c)

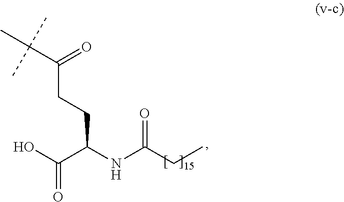

wherein
the dashed line indicates attachment to -D-.

In certain embodiments -AB is of formula (v-d)

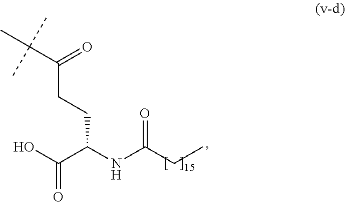

wherein
the dashed line indicates attachment to -D-.

In certain embodiments -AB is of formula (vi-e)

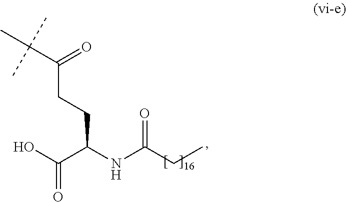

wherein
the dashed line indicates attachment to -D-.

In certain embodiments -AB is of formula (vi-f)

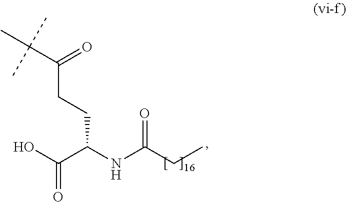

wherein
the dashed line indicates attachment to -D-.

In certain embodiments -AB is of formula (vii)

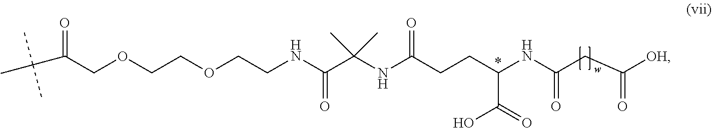

wherein the dashed line indicates attachment to -D-;
w is an integer ranging from and including 14 to 22; and
the stereocenter marked with the asterisk is either in S- or R-configuration.

In certain embodiments -AB is of formula (vii) and w is 14. In certain embodiments -AB is of formula (vii) and w is 15. In certain embodiments -AB is of formula (vii) and w is 16. In certain embodiments -AB is of formula (vii) and w is 17. In certain embodiments -AB is of formula (vii) and w is 18. In certain embodiments -AB is of formula (vii) and w is 19. In certain embodiments -AB is of formula (vii) and w is 20. In certain embodiments -AB is of formula (vii) and w is 21. In certain embodiments -AB is of formula (vii) and w is 22.

In certain embodiments -AB is of formula (vii) and w is 14 and the stereocenter marked with the asterisk is in R-configuration. In certain embodiments -AB is of formula (vii) and w is 15 and the stereocenter marked with the asterisk is in R-configuration. In certain embodiments -AB is of formula (vii) and w is 16 and the stereocenter marked with the asterisk is in R-configuration. In certain embodiments -AB is of formula (vii) and w is 17 and the stereocenter marked with the asterisk is in R-configuration. In certain embodiments -AB is of formula (vii) and w is 18 and the stereocenter marked with the asterisk is in R-configuration.

In certain embodiments -AB is of formula (vii) and w is 19 and the stereocenter marked with the asterisk is in R-configuration. In certain embodiments -AB is of formula (vii) and w is 20 and the stereocenter marked with the asterisk is in R-configuration. In certain embodiments -AB is of formula (vii) and w is 21 and the stereocenter marked with the asterisk is in R-configuration. In certain embodiments -AB is of formula (vii) and w is 22 and the stereocenter marked with the asterisk is in R-configuration.

In certain embodiments -AB is of formula (vii) and w is 14 and the stereocenter marked with the asterisk is in S-configuration. In certain embodiments -AB is of formula (vii) and w is 15 and the stereocenter marked with the asterisk is in S-configuration. In certain embodiments -AB is of formula (vii) and w is 16 and the stereocenter marked with the asterisk is in S-configuration. In certain embodiments -AB is of formula (vii) and w is 17 and the stereocenter marked with the asterisk is in S-configuration. In certain embodiments -AB is of formula (vii) and w is 18 and the stereocenter marked with the asterisk is in S-configuration.

In certain embodiments -AB is of formula (vii) and w is 19 and the stereocenter marked with the asterisk is in S-configuration. In certain embodiments -AB is of formula (vii) and w is 20 and the stereocenter marked with the asterisk is in S-configuration. In certain embodiments -AB is of formula (vii) and w is 21 and the stereocenter marked with the asterisk is in S-configuration. In certain embodiments -AB is of formula (vii) and w is 22 and the stereocenter marked with the asterisk is in S-configuration.

In certain embodiments -AB is of formula (vii-a):

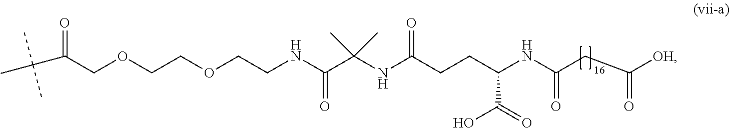

wherein
the dashed line indicates attachment to -D-.

In certain embodiments -AB is of formula (vii-b):

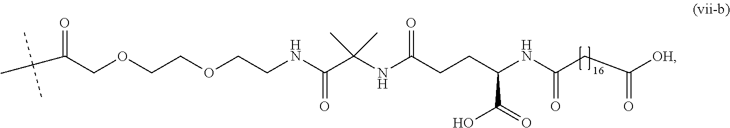

wherein
the dashed line indicates attachment to -D-.

In certain embodiments -AB is of formula (vii-c):

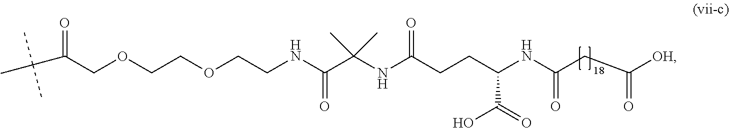

wherein
the dashed line indicates attachment to -D-.

In certain embodiments -AB is of formula (vii-d):

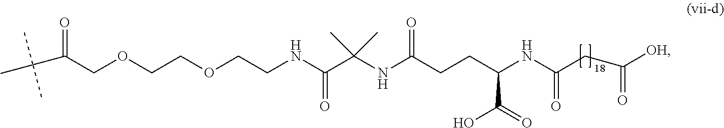

wherein
the dashed line indicates attachment to -D-.

In certain embodiments -AB is of formula (vii-e):

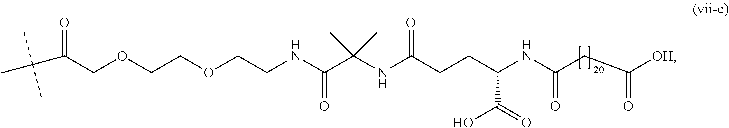

wherein
the dashed line indicates attachment to -D-.

In certain embodiments -AB is of formula (vii-f):

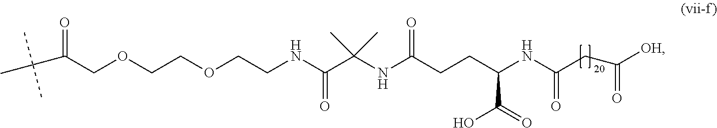

wherein
the dashed line indicates attachment to -D-.

In certain embodiments -AB is of formula (viii)

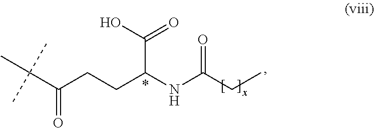

wherein the dashed line indicates attachment to -D-;
w is an integer ranging from and including 14 to 22; and
the stereocenter marked with the asterisk is either in S- or R-configuration.

In certain embodiments -AB is of formula (viii) and x is 14. In certain embodiments -AB is of formula (viii) and x is 15. In certain embodiments -AB is of formula (viii) and x is 16. In certain embodiments -AB is of formula (viii) and x is 17. In certain embodiments -AB is of formula (viii) and x is 18. In certain embodiments -AB is of formula (viii) and x is 19. In certain embodiments -AB is of formula (viii) and x is 20. In certain embodiments -AB is of formula (viii) and x is 21. In certain embodiments -AB is of formula (viii) and x is 22.

In certain embodiments -AB is of formula (viii) and x is 14 and the stereocenter marked with the asterisk is in R-configuration. In certain embodiments -AB is of formula (viii) and x is 15 and the stereocenter marked with the asterisk is in R-configuration. In certain embodiments -AB is of formula (viii) and x is 16 and the stereocenter marked with the asterisk is in R-configuration. In certain embodiments -AB is of formula (viii) and x is 17 and the stereocenter marked with the asterisk is in R-configuration. In certain embodiments -AB is of formula (viii) and x is 18 and the stereocenter marked with the asterisk is in R-configuration. In certain embodiments -AB is of formula (viii) and x is 19 and the stereocenter marked with the asterisk is in R-configuration. In certain embodiments -AB is of formula (viii) and x is 20 and the stereocenter marked with the asterisk is in R-configuration. In certain embodiments -AB is of formula (viii) and x is 21 and the stereocenter marked with the asterisk is in R-configuration. In certain embodiments -AB is of formula (viii) and x is 22 and the stereocenter marked with the asterisk is in R-configuration.

In certain embodiments -AB is of formula (viii) and x is 14 and the stereocenter marked with the asterisk is in S-configuration. In certain embodiments -AB is of formula (viii) and x is 15 and the stereocenter marked with the asterisk is in S-configuration. In certain embodiments -AB is of formula (viii) and x is 16 and the stereocenter marked with the asterisk is in S-configuration. In certain embodiments -AB is of formula (viii) and x is 17 and the stereocenter marked with the asterisk is in S-configuration. In certain embodiments -AB is of formula (viii) and x is 18 and the stereocenter marked with the asterisk is in S-configuration. In certain embodiments -AB is of formula (viii) and x is 19 and the stereocenter marked with the asterisk is in S-configuration. In certain embodiments -AB is of formula (viii) and x is 20 and the stereocenter marked with the asterisk is in S-configuration. In certain embodiments -AB is of formula (viii) and x is 21 and the stereocenter marked with the asterisk is in S-configuration. In certain embodiments -AB is of formula (viii) and x is 22 and the stereocenter marked with the asterisk is in S-configuration.

In certain embodiments -AB is of formula (viii-a)

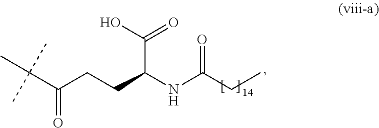

wherein the dashed line indicates attachment to -D-.

In certain embodiments -AB is of formula (viii-b)

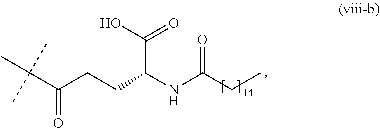

wherein the dashed line indicates attachment to -D-.

In certain embodiments -AB is of formula (viii-c)

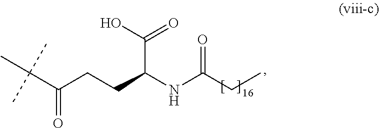

wherein the dashed line indicates attachment to -D-.

In certain embodiments -AB is of formula (viii-d)

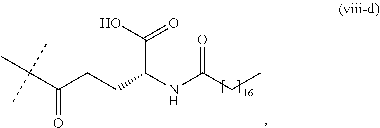

wherein the dashed line indicates attachment to -D-.

In certain embodiments -AB is of formula (viii-e)

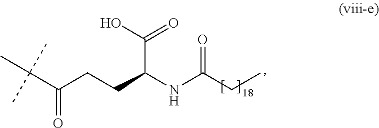

wherein the dashed line indicates attachment to -D-.

In certain embodiments -AB is of formula (viii-f)

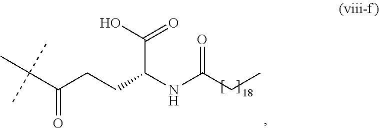

wherein the dashed line indicates attachment to -D-.

In certain embodiments -AB is a peptidic albumin-binding moiety.

If -D- is a peptide or protein drug moiety, such peptidic moiety -AB may be fused to the N- or C-terminus of -D-, either directly or with a peptidic spacer between -D- and -AB.

In certain embodiments -AB is selected from the group consisting of

(SEQ ID NO: 34)
WWEQDRDWDFDVFGGGTP;
(SEQ ID NO: 35)
DICLPRWGCLW,

wherein the cysteines at position 3 and 9 are connected via a disulfide bridge;

(SEQ ID NO: 36)
RLIEDICLPRWGCLWEDD,

wherein the cysteines at position 7 and 13 are connected via a disulfide bridge;

(SEQ ID NO: 37)
LAEAKVLANRELDKYGVSDFYKRLINKAKTVEGVEALKLHILAALP;
(SEQ ID NO: 38)
IAEAKEAANAELDSYGVSDFYKRLIDKAKTVEGVEALKDAILAALP;
and
(SEQ ID NO: 39)
X₁EYEX₂EYE,

wherein X₁ is fluorescein-(AEEA), X₂ is K(palmitate), and AEEA is 2-(2-(2-aminoethoxy)acetyl.

In certain embodiments -AB is of SEQ ID NO:34. In certain embodiments -AB is of SEQ ID NO:35. In certain embodiments -AB is of SEQ ID NO:36. In certain embodiments -AB is of SEQ ID NO:37. In certain embodiments -AB is of SEQ ID NO:38. In certain embodiments -AB is of SEQ ID NO:39.

In certain embodiments -AB is of formula (A-a):

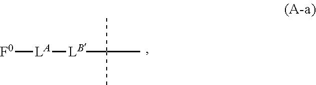

wherein
the dashed line indicates attachment to -D-;
—F⁰ and -L^{A}- are used as defined in formula (A),
L^{B′}- is a polymeric moiety.

The moiety -L^{B′}- is a polymeric moiety, meaning that it comprises at least one polymer moiety. In certain embodiments the one or more polymer moiety has a molecular weight of at least 450 Da. In certain embodiments the one or more polymer moiety has a molecular weight of at least 1 kDa. In certain embodiments the one or more polymer moiety has a molecular weight of at least 1.5 kDa. In certain embodiments the one or more polymer moiety has a molecular weight of at least 2 kDa. In certain embodiments the one or more polymer moiety has a molecular weight of at least 2.5 kDa. In certain embodiments the one or more polymer moiety has a molecular weight of at least 3 kDa. In certain embodiments the one or more polymer moiety has a molecular weight of at least 3.5 kDa. In certain embodiments the one or more polymer moiety has a molecular weight of at least 4 kDa. In certain embodiments the one or more polymer moiety has a molecular weight of at least 5 kDa. In certain embodiments the one or more polymer moiety has a maximum molecular weight of 160 kDa. In certain embodiments the one or more polymer moiety has a maximum molecular weight of 120 kDa. In certain embodiments the one or more polymer moiety has a maximum molecular weight of 100 kDa. In certain embodiments the one or more polymer moiety has a maximum molecular weight of 80 kDa. In certain embodiments the one or more polymer moiety has a maximum molecular weight of 70 kDa. In certain embodiments the one or more polymer moiety has a maximum molecular weight of 60 kDa. In certain embodiments the one or more polymer moiety has a maximum molecular weight of 50 kDa. In certain embodiments the one or more polymer moiety has a maximum molecular weight of 40 kDa. In certain embodiments the one or more polymer moiety has a molecular weight of about 450 Da. In certain embodiments the one or more polymer moiety has a molecular weight of about 1 kDa. In certain embodiments the one or more polymer moiety has a molecular weight of about 1.5 kDa. In certain embodiments the one or more polymer moiety has a molecular weight of about 2 kDa. In certain embodiments the one or more polymer moiety has a molecular weight of about 2.5 kDa. In certain embodiments the one or more polymer moiety has a molecular weight of about 3 kDa. In certain embodiments the one or more polymer moiety has a molecular weight of about 3.5 kDa. In certain embodiments the one or more polymer moiety has a molecular weight of about 4 kDa. In certain embodiments the one or more polymer moiety has a molecular weight of about 4.5 kDa. In certain embodiments the one or more polymer moiety has a molecular weight of about 5 kDa. In certain embodiments the one or more polymer moiety has a molecular weight of about 5.5 kDa. In certain embodiments the one or more polymer moiety has a molecular weight of about 6 kDa. In certain embodiments the one or more polymer moiety has a molecular weight of about 6.5 kDa. In certain embodiments the one or more polymer moiety has a molecular weight of about 7 kDa. In certain embodiments the one or more polymer moiety has a molecular weight of about 7.5 kDa. In certain embodiments the one or more polymer moiety has a molecular weight of about 8 kDa. In certain embodiments the one or more polymer moiety has a molecular weight of about 8.5 kDa. In certain embodiments the one or more polymer moiety has a molecular weight of about 9 kDa. In certain embodiments the one or more polymer moiety has a molecular weight of about 9.5 kDa. In certain embodiments the one or more polymer moiety has a molecular weight of about 10 kDa. It is understood that if -L^{B′}- comprises one polymer moiety the minimum and maximum molecular weights provided above apply to this one polymer moiety and if -L^{B′}- comprises more than one polymer moiety the minimum and maximum molecular weights provided above refer to the minimum and maximum molecular weight of all polymer moieties together.

In certain embodiments the one or more polymer moiety of -L^{B′}- has a Flory radius of at least 1.2 nm. In certain embodiments the one or more polymer moiety of -L^{B′}- has a Flory radius of at least 1.5 nm. In certain embodiments the one or more polymer moiety of -L^{B′}- has a Flory radius of at least 2 nm. In certain embodiments the one or more polymer moiety of-L^{B′}- has a Flory radius of at least 2.5 nm. In certain embodiments the one or more polymer moiety of L^{B′}- has a Flory radius of at least 3 nm. In certain embodiments the one or more polymer moiety of -L^{B′}- has a Flory radius of at least 3.5 nm. In certain embodiments the one or more polymer moiety of -L^{B′}- has a Flory radius of at least 4 nm. In certain embodiments the one or more polymer moiety of -L^{B′}- has a Flory radius of at least 4.5 nm. In certain embodiments the one or more polymer moiety of -L^{B′}- has a Flory radius of at least 5 nm. In certain embodiments the one or more polymer moiety of -L^{B′}- has a Flory radius of no more than 200 nm. In certain embodiments the one or more polymer moiety of -L^{B′}- has a Flory radius of no more than 175 nm. In certain embodiments the one or more polymer moiety of -L^{B′}- has a Flory radius of no more than 150 nm. In certain embodiments the one or more polymer moiety of-L^{B′}- has a Flory radius of no more than 125 nm. In certain embodiments the one or more polymer moiety of -L^{B′}- has a Flory radius of no more than 100 nm. In certain embodiments the one or more polymer moiety of -L^{B′}- has a Flory radius of no more than 75 nm. In certain embodiments the one or more polymer moiety of -L^{B′}- has a Flory radius of no more than 50 nm. In certain embodiments the one or more polymer moiety of -L^{B′}- has a Flory radius of no more than 45 nm. In certain embodiments the one or more polymer moiety of -L^{B′}- has a Flory radius of no more than 40 nm. In certain embodiments the one or more polymer moiety of-L^{B′}- has a Flory radius of no more than 35 nm. In certain embodiments the one or more polymer moiety of -L^{B′}- has a Flory radius of no more than 30 nm. In certain embodiments the one or more polymer moiety of -L^{B′}- has a Flory radius of about 1.2 nm. In certain embodiments the one or more polymer moiety of -L^{B′}- has a Flory radius of about 1.5 nm. In certain embodiments the one or more polymer moiety of -L^{B′}- has a Flory radius of about 2 nm. In certain embodiments the one or more polymer moiety of -L^{B′}- has a Flory radius of about 2.5 nm. In certain embodiments the one or more polymer moiety of -L^{B′}- has a Flory radius of about 3 nm. In certain embodiments the one or more polymer moiety of -L^{B′}- has a Flory radius of about 3.5 nm. In certain embodiments the one or more polymer moiety of -L^{B′}- has a Flory radius of about 4 nm. In certain embodiments the one or more polymer moiety of -L^{B′}- has a Flory radius of about 4.5 nm. In certain embodiments the one or more polymer moiety of -L^{B′}- has a Flory radius of about 5 nm. In certain embodiments the one or more polymer moiety of-L^{B′}- has a Flory radius of about 5.5 nm. In certain embodiments the one or more polymer moiety of -L^{B′}- has a Flory radius of about 6 nm. In certain embodiments the one or more polymer moiety of -L^{B′}- has a Flory radius of about 6.5 nm. In certain embodiments the one or more polymer moiety of -L^{B′}- has a Flory radius of about 7 nm. In certain embodiments the one or more polymer moiety of -L^{B′}- has a Flory radius of about 8.5 nm. In certain embodiments the one or more polymer moiety of -L^{B′}- has a Flory radius of about 9 nm. In certain embodiments the one or more polymer moiety of -L^{B′}- has a Flory radius of about 10 nm. It is understood that if -L^{B′}- comprises one polymer moiety the Flory radius provided above applies to this one polymer moiety and if -L^{B′}- comprises more than one polymer moiety the Flory radius provided above refers to the Flory radius of all polymer moieties together.

-L^{B′}- comprises one or more polymer moiety, such as polymer moiety selected from the group consisting of poly(2-methacryloyl-oxyethyl phosphoyl cholins), poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, alginate, mannans, pectins, rhamnogalacturonans, starches, hydroxyalkyl starches, hydroxyethyl starches and other carbohydrate-based polymers, xylans, and copolymers thereof.

In certain embodiments -L^{B′}- comprises a PEG-based polymer. In certain embodiments -L^{B′}- comprises a hyaluronic acid-based polymer. In certain embodiments -L^{B′}- comprises a random coil polymer. In certain embodiments -L^{B′}- comprises a poly-sarcosine polymer.

In certain embodiments -L^{B′}- of formula (A-a) is of formula (a-3′)

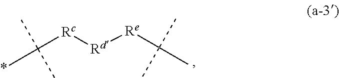

wherein
the unmarked dashed line indicates attachment to -D-;
the dashed line marked with the asterisk indicates attachment to -L^{A};
—R^{c}— is

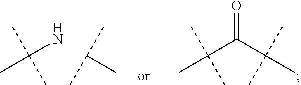

—R^{d′}— is a polymeric moiety; and
—R^{e}— is selected from the group consisting of —CH₂—,

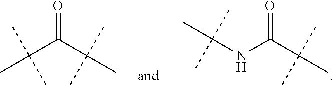

If —R^{b}— of formula (A-a) is

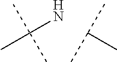

then —R^{c}— of formula (a-3′) is

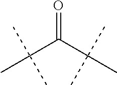

and if —R^{b}— of formula (A-a) is

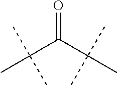

then —R^{c}— of formula (a-3′) is

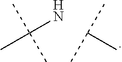

The moiety —R^{d′}— of formula (a-3′) is a polymeric moiety, meaning that it comprises at least one polymer moiety. In certain embodiments the one or more polymer moiety has a molecular weight of at least 450 Da. In certain embodiments the one or more polymer moiety has a molecular weight of at least 1 kDa. In certain embodiments the one or more polymer moiety has a molecular weight of at least 1.5 kDa. In certain embodiments the one or more polymer moiety has a molecular weight of at least 2 kDa. In certain embodiments the one or more polymer moiety has a molecular weight of at least 2.5 kDa. In certain embodiments the one or more polymer moiety has a molecular weight of at least 3 kDa. In certain embodiments the one or more polymer moiety has a molecular weight of at least 3.5 kDa. In certain embodiments the one or more polymer moiety has a molecular weight of at least 4 kDa. In certain embodiments the one or more polymer moiety has a molecular weight of at least 5 kDa. In certain embodiments the one or more polymer moiety has a maximum molecular weight of 160 kDa. In certain embodiments the one or more polymer moiety has a maximum molecular weight of 120 kDa. In certain embodiments the one or more polymer moiety has a maximum molecular weight of 100 kDa. In certain embodiments the one or more polymer moiety has a maximum molecular weight of 80 kDa. In certain embodiments the one or more polymer moiety has a maximum molecular weight of 70 kDa. In certain embodiments the one or more polymer moiety has a maximum molecular weight of 60 kDa. In certain embodiments the one or more polymer moiety has a maximum molecular weight of 50 kDa. In certain embodiments the one or more polymer moiety has a maximum molecular weight of 40 kDa. In certain embodiments the one or more polymer moiety has a molecular weight of about 450 Da. In certain embodiments the one or more polymer moiety has a molecular weight of about 1 kDa. In certain embodiments the one or more polymer moiety has a molecular weight of about 1.5 kDa. In certain embodiments the one or more polymer moiety has a molecular weight of about 2 kDa. In certain embodiments the one or more polymer moiety has a molecular weight of about 2.5 kDa. In certain embodiments the one or more polymer moiety has a molecular weight of about 3 kDa. In certain embodiments the one or more polymer moiety has a molecular weight of about 3.5 kDa. In certain embodiments the one or more polymer moiety has a molecular weight of about 4 kDa. In certain embodiments the one or more polymer moiety has a molecular weight of about 4.5 kDa. In certain embodiments the one or more polymer moiety has a molecular weight of about 5 kDa. In certain embodiments the one or more polymer moiety has a molecular weight of about 5.5 kDa. In certain embodiments the one or more polymer moiety has a molecular weight of about 6 kDa. In certain embodiments the one or more polymer moiety has a molecular weight of about 6.5 kDa. In certain embodiments the one or more polymer moiety has a molecular weight of about 7 kDa. In certain embodiments the one or more polymer moiety has a molecular weight of about 7.5 kDa. In certain embodiments the one or more polymer moiety has a molecular weight of about 8 kDa. In certain embodiments the one or more polymer moiety has a molecular weight of about 8.5 kDa. In certain embodiments the one or more polymer moiety has a molecular weight of about 9 kDa. In certain embodiments the one or more polymer moiety has a molecular weight of about 9.5 kDa. In certain embodiments the one or more polymer moiety has a molecular weight of about 10 kDa. It is understood that if —R^{d′}— of formula (a-3′) comprises one polymer moiety the minimum and maximum molecular weights provided above apply to this one polymer moiety and if —R^{d′}— of formula (a-3′) comprises more than one polymer moiety the minimum and maximum molecular weights provided above refer to the minimum and maximum molecular weight of all polymer moieties together.

In certain embodiments the one or more polymer moiety of —R^{d′}— of formula (a-3′) has a Flory radius of at least 1.2 nm. In certain embodiments the one or more polymer moiety has a Flory radius of at least 1.5 nm. In certain embodiments the one or more polymer moiety has a Flory radius of at least 2 nm. In certain embodiments the one or more polymer moiety has a Flory radius of at least 2.5 nm. In certain embodiments the one or more polymer moiety has a Flory radius of at least 3 nm. In certain embodiments the one or more polymer moiety has a Flory radius of at least 3.5 nm. In certain embodiments the one or more polymer moiety has a Flory radius of at least 4 nm. In certain embodiments the one or more polymer moiety has a Flory radius of at least 4.5 nm. In certain embodiments the one or more polymer moiety has a Flory radius of at least 5 nm. In certain embodiments the one or more polymer moiety has a Flory radius of no more than 200 nm. In certain embodiments the one or more polymer moiety has a Flory radius of no more than 175 nm. In certain embodiments the one or more polymer moiety has a Flory radius of no more than 150 nm. In certain embodiments the one or more polymer moiety has a Flory radius of no more than 125 nm. In certain embodiments the one or more polymer moiety has a Flory radius of no more than 100 nm. In certain embodiments the one or more polymer moiety has a Flory radius of no more than 75 nm. In certain embodiments the one or more polymer moiety has a Flory radius of no more than 50 nm. In certain embodiments the one or more polymer moiety has a Flory radius of no more than 45 nm. In certain embodiments the one or more polymer moiety has a Flory radius of no more than 40 nm. In certain embodiments the one or more polymer moiety has a Flory radius of no more than 35 nm. In certain embodiments the one or more polymer moiety has a Flory radius of no more than 30 nm. In certain embodiments the one or more polymer moiety has a Flory radius of about 1.2 nm. In certain embodiments the one or more polymer moiety has a Flory radius of about 1.5 nm. In certain embodiments the one or more polymer moiety has a Flory radius of about 2 nm. In certain embodiments the one or more polymer moiety has a Flory radius of about 2.5 nm. In certain embodiments the one or more polymer moiety has a Flory radius of about 3 nm. In certain embodiments the one or more polymer moiety has a Flory radius of about 3.5 nm. In certain embodiments the one or more polymer moiety has a Flory radius of about 4 nm. In certain embodiments the one or more polymer moiety has a Flory radius of about 4.5 nm. In certain embodiments the one or more polymer moiety has a Flory radius of about 5 nm. In certain embodiments the one or more polymer moiety has a Flory radius of about 5.5 nm. In certain embodiments the one or more polymer moiety has a Flory radius of about 6 nm. In certain embodiments the one or more polymer moiety has a Flory radius of about 6.5 nm. In certain embodiments the one or more polymer moiety has a Flory radius of about 7 nm. In certain embodiments the one or more polymer moiety has a Flory radius of about 8.5 nm. In certain embodiments the one or more polymer moiety has a Flory radius of about 9 nm. In certain embodiments the one or more polymer moiety has a Flory radius of about 10 nm. It is understood that if —R^{d′}— of formula (a-3′) comprises one polymer moiety the Flory radius provided above applies to this one polymer moiety and if —R^{d′}— of formula (a-3′) comprises more than one polymer moiety the Flory radius provided above refers to the Flory radius of all polymer moieties together.

—R^{d′}— of formula (a-3′) comprises one or more polymer moiety, such as polymer moiety selected from the group consisting of poly(2-methacryloyl-oxyethyl phosphoyl cholins), poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, alginate, mannans, pectins, rhamnogalacturonans, starches, hydroxyalkyl starches, hydroxyethyl starches and other carbohydrate-based polymers, xylans, and copolymers thereof.

In certain embodiments —R^{d′}— of formula (a-3′) comprises a PEG-based polymer. In certain embodiments —R^{d′}— of formula (a-3′) comprises a hyaluronic acid-based polymer. In certain embodiments —R^{d′}— of formula (a-3′) comprises a random coil polymer. In certain embodiments —R^{d′}— of formula (a-3′) comprises a poly-sarcosine polymer.

In certain embodiments the distance between any two moieties -AB of a compound is such that they can bind to two different binding sites on the same albumin moiety or to two different albumin molecules. All or some moieties -AB may bind to different albumin molecules and/or two or more moieties -AB may bind to one albumin molecule. In general, if a compound has x moieties -AB they may bind to up to x albumin moieties. It is understood that not all moieties -AB of a compound may be bound to an albumin moiety at any given time.

Binding to two or more different albumin molecules increases the molecular weight of the compound significantly, which has an advantageous effect on circulation half-life.

In certain embodiments -D- is a protein or peptide drug moiety and -AB is conjugated to a functional group of -D- provided by the N-terminal amine, the C-terminal carboxyl or a side chain of an amino acid residue. In certain embodiments -AB is conjugated to the N-terminal amine functional group of -D-. In certain embodiments -AB is conjugated to the C-terminal carboxyl functional group. In certain embodiments -AB is conjugated to a functional group provided by an amino acid residue of -D-, such as by a lysine, serine, aspartic acid, glutamic acid, arginine, histidine, threonine, glutamine, asparagine, cysteine, proline, tyrosine or tryptophan. In certain embodiments -AB is conjugated to the functional group of the side chain of a lysine of -D-. In certain embodiments -AB is conjugated to the functional group of the side chain of a serine of -D-. In certain embodiments -AB is conjugated to the functional group of the side chain of an aspartic acid of -D-. In certain embodiments -AB is conjugated to the functional group of the side chain of a glutamic acid of -D-. In certain embodiments -AB is conjugated to the functional group of the side chain of an arginine of -D-. In certain embodiments -AB is conjugated to the functional group of the side chain of a histidine of -D-. In certain embodiments -AB is conjugated to the functional group of the side chain of a threonine of -D-. In certain embodiments -AB is conjugated to the functional group of the side chain of a glutamine of -D-. In certain embodiments -AB is conjugated to the functional group of the side chain of an asparagine of -D-. In certain embodiments -AB is conjugated to the functional group of the side chain of a cysteine of -D-. In certain embodiments -AB is conjugated to the functional group of the side chain of a proline of -D-. In certain embodiments -AB is conjugated to the functional group of the side chain of a tyrosine of -D-. In certain embodiments -AB is conjugated to the functional group of the side chain of a tryptophan of -D-.

In certain embodiments -D-AB is selected from the group consisting of semaglutide, liraglutide, ecnoglutide, GZR18, GL0034, tirzepatide, cotadutide, BI-456906, pemvidutide, mazdutide, dapiglutide and retatrutide.

In certain embodiments -D-AB is selected from the group consisting of semaglutide, liraglutide, ecnoglutide, GZR18 and GL0034.

In certain embodiments -D-AB is semaglutide. Semaglutide is a compound of formula

(SEQ ID NO: 40)
HX₁EGTFTSDVSSYLEGQAAKEFIAWLVRGRG,

wherein
X₁ is α-aminoisobutyric acid (Aib); and
the lysine at position 20 is chemically modified through conjugation to the epsilon-amine group of the lysine side-chain with

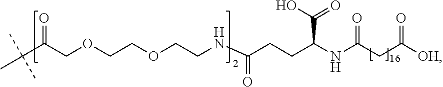

wherein
the dashed line indicates attachment to the epsilon-amine group of the lysine side chain of the lysine at position 20.

Semaglutide may be prepared using methods known to those skilled in the art, such as those described in WO2006/097537.

In certain embodiments -D-AB is liraglutide. Liraglutide is a compound of formula

(SEQ ID NO: 41)
HAEGTFTSDVSSYLEGQAAKEFIAWLVRGRG,

wherein
the lysine at position 20 is chemically modified through conjugation to the epsilon-amine group of the lysine side chain with

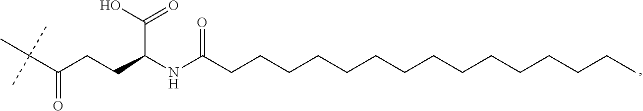

wherein
the dashed line indicates attachment to the epsilon-amine group of the lysine side chain of the lysine at position 20.

In certain embodiments -D-AB is ecnoglutide. Ecnoglutide is a compound of formula

(SEQ ID NO: 42)
HVEGTFTSDVSSYLEEQAAREFIKWLVRGRG,

wherein
the lysine at position 24 is chemically modified through conjugation to the epsilon-amine group of the lysine side chain with

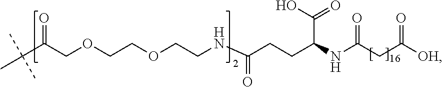

wherein the dashed line indicates attachment to the epsilon-amine group of the lysine side chain of the lysine at position 24.

In certain embodiments -D-AB is GZR18. GZR18 is a compound of formula

(SEQ ID NO: 43)
HGEGTFTSDVSSYLEGQAAKEFIAWLVRGRG,

wherein
the lysine at position 20 is chemically modified through conjugation to the epsilon-amine group of the lysine side-chain with

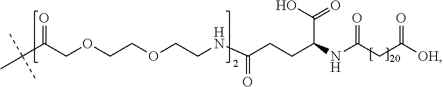

wherein the dashed line indicates attachment to the epsilon amine group of the lysine side chain of the lysine at position 20.

In certain embodiments -D-AB is GL0034. GL0034 also known as utreglutide and is a compound of formula

(SEQ ID NO: 44)
HX₁EGTFTSDVSSYLEGQAAKEFIAWLVRGRGL,

wherein
X₁ is Aib; and
the lysine at position 20 is chemically modified through conjugation to the epsilon-amine group of the lysine side-chain with

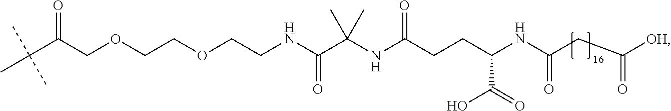

wherein the dashed line indicates attachment to the epsilon amine group of the lysine side chain of the lysine at position 20.

In certain embodiments -D or -D-AB is a dual GLP-1 receptor agonist selected from the group consisting of tirzepatide, cotadutide, BI-456906, pemvidutide and mazdutide.

In certain embodiments -D-AB is a dual GLP-1 receptor agonist that activates the GLP-1 receptor and the GIP receptor. An example for such dual GLP-1 receptor agonist is tirzepatide.

In certain embodiments -D-AB is tirzepatide. Tirzepatide is a compound of formula

(SEQ ID NO: 45)
YX₁EGTFTSDYSIX₂LDKIAQKAFVQWLIAGGPSSGAPPPS,

wherein
X₁ is Aib;
X₂ is Aib;
the lysine at position 20 is chemically modified through conjugation to the epsilon-amine group of the lysine side-chain with ([2-(2-amino-ethoxy)-ethoxy]-acetyl)₂-(γGlu)₁-CO—(CH₂)₁₈—CO₂H; and
the C-terminal serine, i.e. the serine at position 39, is amidated as a C-terminal primary amide.

The moiety ([2-(2-amino-ethoxy)-ethoxy]-acetyl)₂-(γGlu)₁-CO—(CH₂)₁₈—CO₂H has the following structure

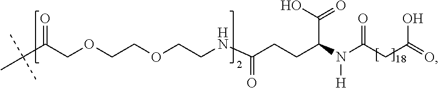

wherein the dashed line indicates attachment to -D-.

In certain embodiments -D-AB is a dual GLP-1 receptor agonist that activates the GLP-1 receptor and the glucagon receptor selected from the group consisting of cotadutide, BI-456906, pemvidutide and mazdutide.

In certain embodiments -D-AB is cotadutide. Cotadutide is a compound of formula

(SEQ ID NO: 46)
HSQGTFTSDKSEYLDSERARDFVAWLEAGG,

wherein
the lysine at position 10 is chemically modified through conjugation to the epsilon-amine group of the lysine side-chain with γ-Glu-palmitoyl.

The moiety γ-Glu-palmitoyl has the following structure:

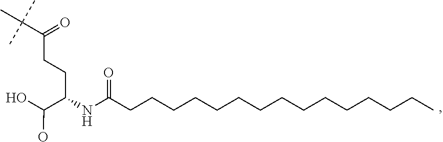

wherein the dashed line indicates attachment to -D-.

In certain embodiments -D-AB is BI-456906. BI-456906 is also known as survodutide and is a compound of formula

(SEQ ID NO: 47)
HX₁QGTFTSDYSKYLDERAAKDFIKWLESA,

wherein
X₁ is 1-amino-cyclobutanecarboxylic acid (Ac4c);
the lysine at position 24 is chemically modified through conjugation to the epsilon-amine group of the lysine side-chain with [17-carboxy-heptadecanoyl]-isoGlu-GSGSGG; and
the C-terminal alanine, i.e. the alanine at position 29, is amidated as a C-terminal primary amide.

The moiety [17-carboxy-heptadecanoyl]-isoGlu-GSGSGG has the following structure:

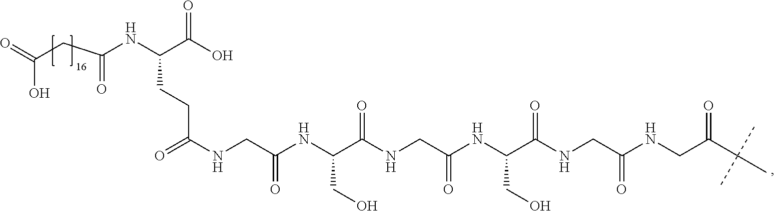

wherein the dashed line indicates attachment to -D-.

In certain embodiments -D-AB is pemvidutide. Pemvidutide is a compound of formula

(SEQ ID NO: 48)
HX₁QGTFTSDYSKYLDEKAAKEFIQWLLQT,

wherein
X₁ is Aib;
the glutamic acid at position 16 and the lysine at position 20 are connected via a lactam bridge;
the lysine at position 17 is chemically modified through conjugation to the epsilon-amine group of the lysine side-chain with glucuronic acid C-18, which is a moiety of formula

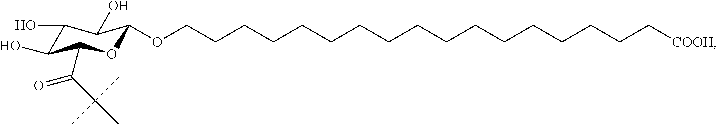

wherein
the dashed line indicates attachment to the epsilon-amine group of the lysine at position 17; and
the C-terminal threonine, i.e. the threonine at position 29, is amidated as a C-terminal primary amide

In certain embodiments -D-AB is mazdutide. Mazdutide is a compound of formula

(SEQ ID NO: 49)
HX₁QGTFTSDYSKYLDEKKAKEFVEWLLEGGPSSG,

wherein
X₁ is Aib,
the lysine at position 20 is chemically modified by conjugation of the epsilon-amine group of the lysine side chain with ([2-(2-amino-ethoxy)-ethoxy]-acetyl)₂-(γ-Glu)—CO—(CH₂)₁₈—CO₂H; and
the C-terminal glycine, i.e. the glycine at position 34, is amidated.

The moiety ([2-(2-amino-ethoxy)-ethoxy]-acetyl)₂-(γ-Glu)—CO—(CH₂)₁₈—CO₂H has the following structure:

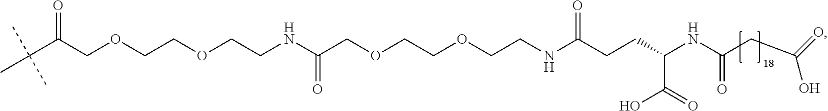

wherein the dashed line indicates attachment to -D-.

In certain embodiments -D-AB is a dual GLP-1 receptor agonist that activates the GLP-1 receptor and the GLP-2 receptor, such as dapiglutide. Dapiglutide is a compound of formula

(SEQ ID NO: 50)
HX₁EGSFTSELATILDKQAARDFIAWLIQHKITD,

wherein
X₁ is Aib; and
the lysine in position 16 is chemically modified by conjugation of the epsilon-amino-group of the lysine side chain with [17-carboxy-heptadecanoyl]-isoGlu.

The moiety [17-carboxy-heptadecanoyl]-isoGlu has the following structure:

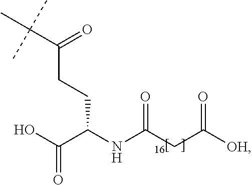

wherein the dashed line indicates attachment to -D-.

In certain embodiments -D-AB is retatrutide, which is a triple GLP-1 receptor agonist that activates the GLP-1 receptor, the GIP receptor and the GCG receptor. Retatrutide is a compound of formula

(SEQ ID NO: 51)
YX₁QGTFTSDYSIX₂LDKKAQX₃AFIEYLLEGGPSSGAPPPS,

wherein
X₁ is Aib;
X₂ is α-methyl-leucine (αMeL);
X₃ is Aib;
the lysine at position 17 is chemically modified by conjugation of the epsilon-amine group of the lysine side chain with (2-[2-(2-amino-ethoxy)-ethoxy]-acetyl)-(γGlu)—CO—(CH₂)₁₈—CO₂H; and
the C-terminal serine, i.e. the serine at position 39, is amidated.

Retatrutide can also be described as Y-Aib-QGTFTSDYSI-αMeL-LDKK ((2-[2-(2-amino-ethoxy)-ethoxy]-acetyl)-(γGlu)—CO—(CH₂)₁₈—CO₂H) AQ-Aib-AFIEYLLEGGPSSGAPPPS-NH₂ (SEQ ID NO:51).

The moiety (2-[2-(2-amino-ethoxy)-ethoxy]-acetyl)-(γGlu)—CO—(CH₂)₁₈—CO₂H has the following structure:

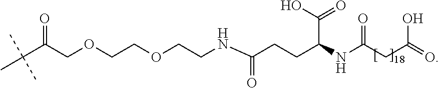

In certain embodiments -D-AB is noiiglutide, also known as SHR20004.

In certain embodiments -D-AB is ZT002.

In certain embodiments -D-AB is selected from the group consisting of semaglutide, liraglutide, ecnoglutide, GZR18, GL0034, tirzepatide, cotadutide, BI-456906, pemvidutide, mazdutide, dapiglutide and retatrutide.

In certain embodiments -D-AB is of formula (b-1)

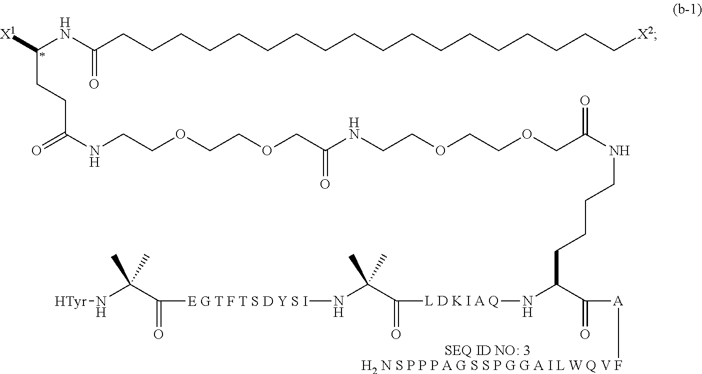

wherein
X¹— is

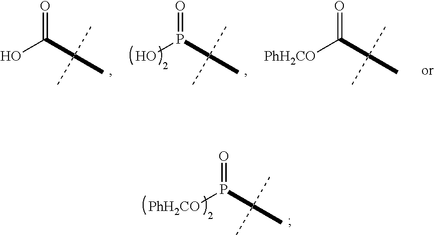

and
—X² is

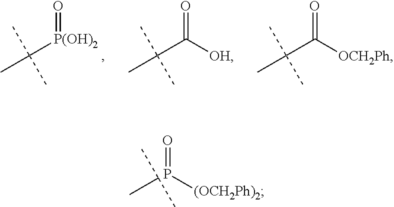

with Ph being phenyl.

The moiety -D- of formula b-1 has the sequence of SE ID NO:57

YX₁EGTFTSDYSIX₂LDKIAQKAFVQWLIAGGPSSGAPPPS,

with X₁ and X₂ being Aib.

The compounds of formula (b-1) are disclosed in WO2022159395 and WO2023/044290, the content of which is herewith incorporated by reference in its entirety.

In certain embodiments -D-AB is of formula (b-1) and X¹— is

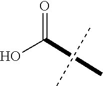

### and —X² is

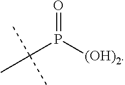

In certain embodiments D-AB is of formula (b-1) and X¹— is

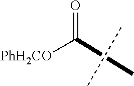

### and —X² is

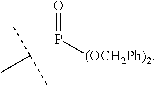

In certain embodiments D-AB is of formula (b-1) and X¹— is

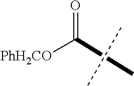

### and —X² is

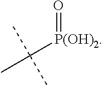

In certain embodiments D-AB is of formula (b-1) and X¹— is

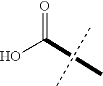

### and —X² is

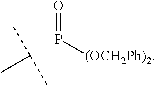

In certain embodiments D-AB is of formula (b-1) and X¹— is

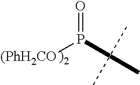

### and —X² is

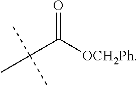

In certain embodiments D-AB is of formula (b-1) and X¹— is

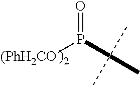

### and —X² is

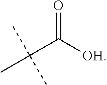

In certain embodiments D-AB is of formula (b-1) and X¹— is

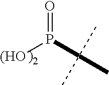

### and —X² is

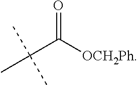

In certain embodiments D-AB is of formula (b-1) and X¹— is

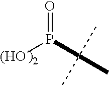

### and —X² is

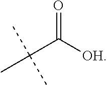

In certain embodiments D-AB is of formula (b-1) and X¹— is

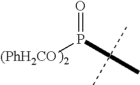

### and —X² is

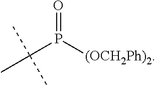

In certain embodiments D-AB is of formula (b-1) and X¹— is

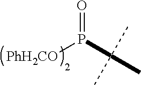

### and —X² is

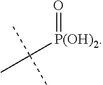

In certain embodiments D-AB is of formula (b-1) and X¹— is

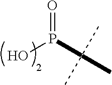

### and —X² is

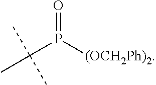

In certain embodiments D-AB is of formula (b-1) and X¹— is

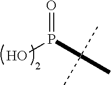

### and —X² is

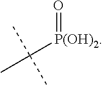

In certain embodiments -D-AB is a compound of formula

(SEQ ID NO: 52)
k(γE-(miniPEG)₂-γE-COC₁₆H₃₂CO₂H)(N-Me)
GSVSEIQLMHNLGKHLNSMERVEW
LRKKLQDVHK(γE-(miniPEG)₂-γE-COC₁₆H₃₂CO₂H)-OH,

wherein
k is d-Lys;
γE is the l-isomer of gamma, glutamic acid;
miniPEG is COCH₂OCH₂CH₂OCH₂CH₂NH;
COC₁₆H₃₂CO₂H is C18 diacid;
(N-Me)G is sarcosine;
K is l-isomer of lysine; and
—OH designates the C-terminal amino acid has a terminal carboxylic acid.

In certain embodiments -D-AB is a compound of formula

(SEQ ID NO: 53)
k(γE-(miniPEG)₂-γE-COC₁₆H₃₂CO₂H)(N-Me)
GSVSEIQLMHNLGKHLNSMERVEW
LRKKLQDVHK(γE-(miniPEG)₂-γE-COC₁₆H₃₂CO₂H)-OH,

wherein
k is d-Lys;
γE is the l-isomer of gamma, glutamic acid;
(miniPEG)₂ is COCH₂OCH₂CH₂OCH₂CH₂NH;
COC₁₆H₃₂CO₂H is C18 diacid;
(N-Me)G is sarcosine;
K is l-isomer of lysine; and
—OH designates the C-terminal amino acid has a terminal carboxylic acid.

If -D- is a peptide or protein drug moiety, -L¹- is either conjugated to a functional group of a side chain of an amino acid residue of -D-, to the N-terminal amine functional group or to the C-terminal carboxyl functional group of -D- or to a nitrogen atom in the backbone chain of -D-.

If -D- is a peptide or protein drug moiety, -L¹- is in certain embodiments conjugated to a functional group of -D- selected from the group consisting of carboxylic acid, primary amine, secondary amine, maleimide, thiol, sulfonic acid, carbonate, carbamate, hydroxyl, aldehyde, ketone, hydrazine, isocyanate, isothiocyanate, phosphoric acid, phosphonic acid, haloacetyl, alkyl halide, acryloyl, aryl fluoride, hydroxyamine, sulfate, disulfide, vinyl sulfone, vinyl ketone, diazoalkane, oxirane, guanidine and aziridine. If -D- is a peptide or protein drug moiety, -L¹- is in certain embodiments conjugated to a functional group of -D- selected from the group consisting of hydroxyl, primary amine, secondary amine and guanidine. If -D- is a peptide or protein drug moiety, -L¹- is in certain embodiments conjugated to a functional group of -D- selected from the group consisting of primary amine and secondary amine. If -D- is a peptide or protein drug moiety, -L¹- is in certain embodiments conjugated to a primary amine of -D-.

If -D- is a peptide or protein drug moiety, -L¹- may be conjugated to a functional group of the side chain of an amino acid residue of -D-, which may be a proteinogenic amino acid residue or a non-proteinogenic amino acid residue.

If -D- is a peptide or protein drug moiety, -L¹- is in certain embodiments conjugated to a functional group of the side chain of a proteinogenic amino acid residue of -D-. In certain embodiments such amino acid residue is selected from the group consisting of histidine, lysine, tryptophan, serine, threonine, tyrosine, aspartic acid, glutamic acid and arginine. In certain embodiments such amino acid residue is selected from the group consisting of lysine, aspartic acid, arginine and serine. In certain embodiments such amino acid residue is selected from the group consisting of lysine, arginine and serine.

If -D- is a peptide or protein drug moiety, -L¹- is in certain embodiments conjugated to a functional group of a lysine residue of -D-. If -D- is a peptide or protein drug moiety, -L¹- is in certain embodiments conjugated to a functional group of a histidine residue of -D-. If -D- is a peptide or protein drug moiety, -L¹- is in certain embodiments conjugated to a functional group of a tryptophan residue of -D-. If -D- is a peptide or protein drug moiety, -L¹- is in certain embodiments conjugated to a functional group of a serine residue of -D-. If -D- is a peptide or protein drug moiety, -L¹- is in certain embodiments conjugated to a functional group of a threonine residue of -D-. If -D- is a peptide or protein drug moiety, -L¹- is in certain embodiments conjugated to a functional group of a tyrosine residue of -D-. If -D- is a peptide or protein drug moiety, -L¹- is in certain embodiments conjugated to a functional group of an aspartic acid residue of -D-. If -D- is a peptide or protein drug moiety, -L¹- is in certain embodiments conjugated to a functional group of a glutamic acid residue of -D-. If -D- is a peptide or protein drug moiety, -L¹- is in certain embodiments conjugated to a functional group of an arginine residue of -D-.

If -D- is a peptide or protein drug moiety, -L¹- is in certain embodiments conjugated to a functional group of the side chain of a non-proteinogenic amino acid residue of -D-.

It is understood that not every peptide or protein drug moiety -D- may comprise all of these amino acid residues.

If -D- is a peptide or protein drug moiety, -L¹- is in certain embodiments conjugated to the N-terminal amine functional group of -D-.

If -D- is a peptide or protein drug moiety, -L¹- is in certain embodiments conjugated to the C-terminal functional group of -D-.

The moiety -L¹- may be connected to -D- through any type of linkage, provided that it is reversible. In certain embodiments -L¹- is connected to -D- through a linkage selected from the group consisting of amide, ester, carbamate, acetal, aminal, imine, oxime, hydrazone, disulfide and acylguanidine. In certain embodiments -L¹- is connected to -D- through a linkage selected from the group consisting of amide, ester, carbamate and acylguanidin. It is understood that some of these linkages per se are not reversible, but that in the present invention neighboring groups present in -L¹- render these linkages reversible.

In certain embodiments -L¹- is connected to -D- through an amide linkage. In certain embodiments -L¹- is connected to -D- through a carbamate linkage. In certain embodiments -L¹- is connected to -D- through an ester linkage. In certain embodiments -L¹- is connected to -D- through an acylguanidine linkage.

The moiety -L¹- is a reversible prodrug linker from which the drug, i.e., H-D-AB, is released in its free form, i.e. it is a traceless prodrug linker. It is understood that the “H-” in “H-D-AB” is a hydrogen. Suitable prodrug linkers are known in the art, such as for example the reversible prodrug linker moieties disclosed in WO 2005/099768 A2, WO 2006/136586 A2, WO 2011/089216 A1 and WO 2013/024053 A1, which are incorporated by reference herewith.

In certain embodiments -L¹- is disclosed in WO 2009/095479 A2. Accordingly, in certain embodiments the moiety -L¹- is of formula (II):

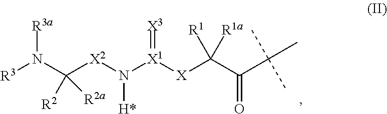

wherein the dashed line indicates attachment to a nitrogen, hydroxyl or thiol of -D-;
—X— is selected from the group consisting of —C(R⁴R^{4a})—; —N(R⁴)—; —O—; —C(R⁴R^{4a})—, C(R⁵R^{5a})—; —C(R⁵R^{5a})—C(R⁴R^{4a})—; —C(R⁴R^{4a})—N(R⁶)—; —N(R⁶)—C(R⁴R^{4a})—; C(R⁴R^{4a})—O—; —O—C(R⁴R^{4a})—; and —C(R⁷R^{7a})—;
X¹ is selected from the group consisting of C; and S(O);
—X²— is selected from the group consisting of —C(R⁸R^{8a})—; and —C(R⁸R^{8a})—C(R⁹R^{9a})—;
═X³ is selected from the group consisting of ═O; ═S; and =N—CN;
—R¹, —R^{1a}, —R², —R^{2a}, —R⁴, —R^{4a}, —R⁵, —R^{5a}, —R⁶, —R⁸, —R^{8a}, —R⁹, and —R^{9a} are independently selected from the group consisting of —H; and C₁₋₆ alkyl;
—R³, and —R^{3a} are independently selected from the group consisting of —H; and C₁₋₆ alkyl, provided that in case one of —R³, —R^{3a} or both are other than —H they are connected to N to which they are attached through an SP³-hybridized carbon atom;
—R⁷ is selected from the group consisting of —N(R¹⁰R^{10a}); and —NR¹⁰—(C═O)—R¹¹; —R^{7a}, —R¹⁰, —R^{10a}, and —R¹¹ are independently of each other selected from the group consisting of —H; and C₁₋₆ alkyl;
optionally, one or more of the pairs —R^{1a}/—R^{4a}, —R^{1a}/—R^{5a}, —R^{1a}/—R^{7a}, —R^{4a}/—R^{5a}, and —R^{8a}/—R^{9a} form a chemical bond;
optionally, one or more of the pairs —R^{1a}/—R^{1a}, —R²/—R^{2a}, —R⁴/—R^{4a}, —R⁵/—R^{5a}, —R⁵/—R^{5a}, and —R⁹/—R^{9a} are joined together with the atom to which they are attached to form a C₃₋₁₀ cycloalkyl; or 3- to 10-membered heterocyclyl;
optionally, one or more of the pairs —R¹/—R⁴, —R¹/—R⁵, —R¹/—R⁶, —R¹/—R^{7a}, —R⁴/—R⁵, —R⁴/—R⁶, —R⁸/—R⁹, and —R²/—R³ are joined together with the atoms to which they are attached to form a ring A;
optionally, R³/R^{3a} are joined together with the nitrogen atom to which they are attached to form a 3- to 10-membered heterocycle;
A is selected from the group consisting of phenyl; naphthyl; indenyl; indanyl; tetralinyl; C₃₋₁₀ cycloalkyl; 3- to 10-membered heterocyclyl; and 8- to 11-membered heterobicyclyl; and wherein -L¹- is substituted with at least one -L²-Z and wherein -L¹- is optionally further substituted, provided that the hydrogen marked with the asterisk in formula (II) is not replaced by -L²-Z or a substituent.

In certain embodiments -L¹- of formula (II) is substituted with one moiety -L²-Z.

In certain embodiments -L¹- of formula (II) is not further substituted.

It is understood that if —R³/—R^{3a} of formula (II) are joined together with the nitrogen atom to which they are attached to form a 3- to 10-membered heterocycle, only such 3- to 10-membered heterocycles may be formed in which the atoms directly attached to the nitrogen are SP³-hybridized carbon atoms. In other words, such 3- to 10-membered heterocycle formed by —R³/—R^{3a} together with the nitrogen atom to which they are attached has the following structure:

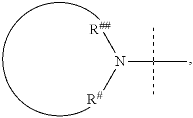

wherein
the dashed line indicates attachment to the rest of -L¹-;
the ring comprises 3 to 10 atoms comprising at least one nitrogen; and
R^{#} and R^{##} represent an sp³-hybridized carbon atom.

It is also understood that the 3- to 10-membered heterocycle may be further substituted.

Exemplary embodiments of suitable 3- to 10-membered heterocycles formed by —R³/—R^{3a} of formula (II) together with the nitrogen atom to which they are attached are the following:

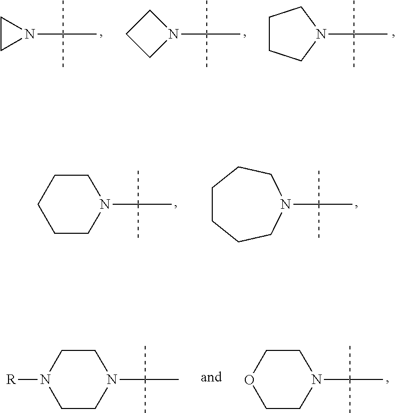

wherein
dashed lines indicate attachment to the rest of the molecule; and
—R is selected from the group consisting of —H and C₁₋₆ alkyl.

-L¹- of formula (II) may optionally be further substituted. In general, any substituent may be used as far as the cleavage principle is not affected, i.e., the hydrogen marked with the asterisk in formula (II) is not replaced and the nitrogen of the moiety

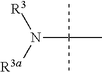

of formula (II) remains part of a primary, secondary or tertiary amine, i.e., —R³ and —R^{3a} are independently of each other —H or are connected to —N< through an sp³-hybridized carbon atom.

In one embodiment —R¹ or —R^{1a} of formula (II) is substituted with -L²-Z. In another embodiment —R² or —R^{2a} of formula (II) is substituted with -L²-Z. In another embodiment —R³ or —R^{3a} of formula (II) is substituted with -L²-Z. In another embodiment —R⁴ of formula (II) is substituted with -L²-Z. In another embodiment —R⁵ or —R^{5a} of formula (II) is substituted with -L²-Z. In another embodiment —R⁶ of formula (II) is substituted with -L²-Z. In another embodiment —R⁷ or —R^{7a} of formula (II) is substituted with -L²-Z. In another embodiment —R⁸ or —R^{8a} of formula (II) is substituted with -L²-Z. In another embodiment —R⁹ or —R^{9a} of formula (II) is substituted with -L²-Z. In another embodiment —R¹⁰ is substituted with -L²-Z. In another embodiment —R¹¹ is substituted with -L²-Z. In certain embodiments —R³ of formula (II) is substituted with -L²-Z.

In certain embodiments —X— of formula (II) is selected from the group consisting of —C(R⁴R^{4a})—, —N(R⁴)— and —C(R⁷R^{7a})—. In certain embodiments —X— of formula (II) is —C(R⁴R^{4a})—. In certain embodiments —X— of formula (II) is —C(R⁷R^{7a})—;

In certain embodiments —R⁷ of formula (II) is —NR¹⁰—(C═O)—R¹¹.

In certain embodiments —R^{7a} of formula (II) is selected from —H, methyl and ethyl. In certain embodiments —R^{7a} of formula (II) is —H.

In certain embodiments —R¹⁰ is selected from —H, methyl and ethyl. In certain embodiments —R¹⁰ is methyl.

In certain embodiments —R¹¹ is selected from —H, methyl and ethyl. In certain embodiments —R¹¹ is —H. In certain embodiments —R¹¹ is substituted with -L²-Z.

In certain embodiments —X— of formula (II) is —N(R⁴)—.

In certain embodiments —R⁴ is selected from the group consisting of —H, methyl and ethyl. In certain embodiments —R⁴ is —H.

In certain embodiments X¹ of formula (II) is C.

In certain embodiments ═X³ of formula (II) is ═O.

In certain embodiments —X²— of formula (II) is —C(R⁸R^{8a})—;

In certain embodiments —R⁸ and —R^{8a} of formula (II) are independently selected from the group consisting of —H, methyl and ethyl. In certain embodiments at least one of —R⁸ and —R^{8a} of formula (II) is —H. In certain embodiments both —R⁸ and —R^{8a} of formula (II) are —H.

In certain embodiments —R¹ and —R^{1a} of formula (II) are independently selected from the group consisting of —H, methyl and ethyl.

In certain embodiments at least one of —R¹ and —R^{1a} of formula (II) is —H. In certain embodiments —R¹ and —R^{1a} of formula (II) are —H.

In certain embodiments at least one of —R¹ and —R^{1a} of formula (II) is methyl. In certain embodiments both —R¹ and —R^{1a} of formula (II) are methyl.

In certain embodiments —R² and —R^{2a} of formula (II) are independently selected from the group consisting of —H, methyl and ethyl. In certain embodiments at least one of —R² and —R^{2a} of formula (II) is —H. In certain embodiments both —R² and —R^{2a} of formula (II) are H.

In certain embodiments —R³ and —R^{3a} of formula (II) are independently selected from the group consisting of —H, methyl, ethyl, propyl and butyl.

In certain embodiments at least one of —R³ and —R^{3a} of formula (II) is methyl. In certain embodiments —R³ of formula (II) is methyl and —R^{3a} of formula (II) is —H.

In certain embodiments —R³ and —R^{3a} of formula (II) are both —H.

In certain embodiments -D- is connected to -L¹- through a nitrogen by forming an amide bond.

In certain embodiments -L¹- is of formula (II), wherein X is —C(R⁷R^{7a})—; X¹ is C; —X²— is —C(R⁸R^{8a})—C(R⁹R^{9a})—; ═X³ is ═O; —R¹ and —R^{1a} are —H; —R² and —R^{2a} are —H; —R³ and —R^{3a} are methyl; —R⁷ is —N(R¹⁰R^{10a}); —R^{7a} is —H; —R⁸, —R^{8a}, —R⁹ and —R^{9a} are —H; and —R¹⁰ is methyl and —R^{10a} is —H.

In certain embodiments -L¹- is of formula (IIa)

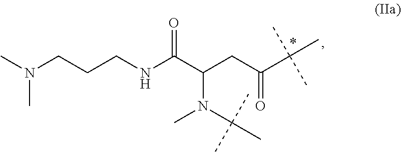

wherein
the dashed line marked with the asterisk indicates attachment to a nitrogen of -D-; and
the unmarked dashed line indicates attachment to -L²-.

In certain embodiments -L¹- is of formula (IIa-a)

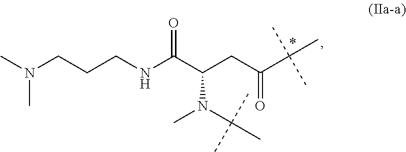

wherein
the dashed line marked with the asterisk indicates attachment to a nitrogen of -D-; and
the unmarked dashed line indicates attachment to -L²-.

In certain embodiments -L¹- is of formula (IIa-b)

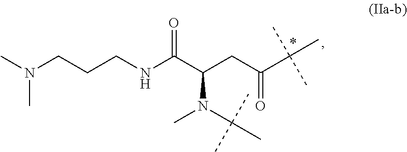

wherein
the dashed line marked with the asterisk indicates attachment to a nitrogen of -D-; and
the unmarked dashed line indicates attachment to -L²-

The moiety of formula (IIa), (IIa-a) and (IIa-b) is connected to -D- via an amide bond formed by a nitrogen of an amine functional group and the carbonyl to the left of the dashed line marked with the asterisk.

In certain embodiments -L¹- is of formula (II), wherein X is —C(R⁷R^{7a})—; X¹ is C; —X²— is —C(R⁸R^{8a})—C(R⁹R^{9a})—; ═X³ is ═O; —R¹ and —R^{1a} are —H; —R² and —R^{2a} are —H; —R³ and —R^{3a} are methyl; —R⁷ is —NR¹⁰—(C═O)—R¹¹; —R^{7a} is —H; —R⁸, —R^{8a}, —R⁹ and —R^{9a} are —H; and —R¹⁰ is methyl and —R¹¹ is —H.

In certain embodiments -L¹- is of formula (II), wherein X is —C(R⁷R^{7a})—; X¹ is C; —X²— is —C(R⁸R^{8a})—C(R⁹R^{9a})—; ═X³ is ═O; —R¹ and —R^{1a} are —H; —R² and —R^{2a} are —H; —R³ and —R^{3a} are methyl; —R⁷ is —NR¹⁰—(C═O)—R¹¹; —R^{7a} is —H; —R⁸, —R^{8a}, —R⁹ and —R^{9a} are —H; and —R¹⁰ is —H and —R¹¹ is —H.

In certain embodiments -L¹- is of formula (IIab)

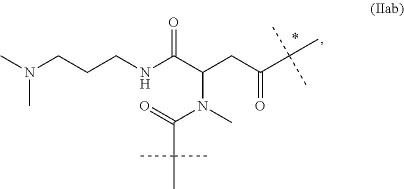

wherein
the dashed line marked with the asterisk indicates attachment to a nitrogen of -D-; and
the unmarked dashed line indicates attachment to -L²-.

In certain embodiments -L¹- is of formula (IIab-a)

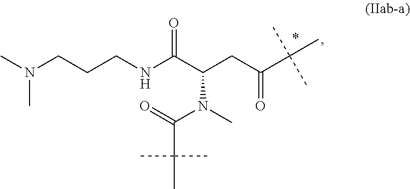

wherein
the dashed line marked with the asterisk indicates attachment to a nitrogen of -D-; and
the unmarked dashed line indicates attachment to -L²-.

In certain embodiments -L¹- is of formula (IIab-b)

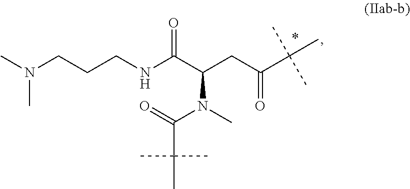

wherein
the dashed line marked with the asterisk indicates attachment to a nitrogen of -D-; and
the unmarked dashed line indicates attachment to -L²-.

The moiety of formula (IIab), (IIab-a) and (IIab-b) is connected to -D- via an amide bond formed by a nitrogen of an amine functional group and the carbonyl to the left of the dashed line marked with the asterisk.

In certain embodiments -L¹- is disclosed in WO2016/020373A1. Accordingly, in certain embodiments the moiety -L¹- is of formula (III):

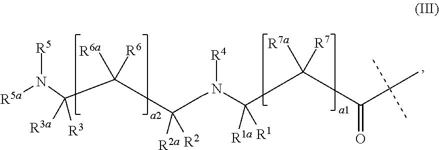

wherein
the dashed line indicates attachment to a primary or secondary amine or hydroxyl of -D- through an amide or ester linkage, respectively;
—R¹, —R^{1a}, —R², —R^{2a}, —R³ and —R^{3a} are independently of each other selected from the group consisting of —H, —C(R⁸R^{8a}R^{8b}), —C(═O)R⁸, —C—N, —C(═NR⁸)R^{8a}, —CR⁸(═CR^{8a}R^{8b}), —C≡CR⁸ and -T;
—R⁴, —R⁵ and —R^{5a} are independently of each other selected from the group consisting of —H, —C(R⁹R^{9a}R^{9b}) and -T;
a1 and a2 are independently of each other 0 or 1;
each —R⁶, —R^{6a}, —R⁷, —R^{7a}, —R⁸, —R^{8a}, —R^{8b}, —R⁹, —R^{9a}, and —R^{9b} are independently of each other selected from the group consisting of —H, halogen, —CN, —COOR¹⁰, —OR¹⁰, —C(O)R¹⁰, —C(O)N(R¹⁰R^{10a}), —S(O)₂N(R¹⁰R^{10a}), —S(O) N(R¹⁰R^{10a}), —S(O)₂R¹⁰, —S(O)R¹⁰, —N(R¹⁰)S(O)₂N(R^{10a}R^{10b}), —SR¹⁰, —N(R¹⁰R^{10a}), —NO₂, —OC(O)R¹⁰, —N(R¹⁰)C(O)R^{10a}, —N(R¹⁰)S(O)₂R^{10a}, —N(R¹⁰)S(O)R^{10a}, —N(R¹⁰)C(O)OR^{10a}, —N(R¹⁰)C(O)N(R^{10a}R^{10b}),—OC(O)N(R¹⁰R^{10a}), -T, C₁₋₂₀ alkyl, C₂₋₂₀ alkenyl, and C₂₋₂₀ alkynyl; wherein -T, C₁₋₂₀ alkyl, C₂₋₂₀ alkenyl, and C₂₋₂₀ alkynyl are optionally substituted with one or more —R¹¹, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)(R^{12a})—, —N(R¹²)C(O)N(R^{12a})—, and —OC(O)N(R¹²)—;
each —R¹⁰, —R^{10a}, and —R^{10b} is independently selected from the group consisting of —H, -T, C₁₋₂₀ alkyl, C₂₋₂₀ alkenyl, and C₂₋₂₀ alkynyl; wherein -T, C₁₋₂₀ alkyl, C₂₋₂₀ alkenyl, and C₂₋₂₀ alkynyl are optionally substituted with one or more —R¹¹, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)(R^{12a})—, —N(R¹²)C(O)N(R^{12a})—, and —OC(O)N(R¹²)—;
each T is independently of each other selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T is independently optionally substituted with one or more —R¹¹, which are the same or different;
each —R¹¹ is independently of each other selected from halogen, —CN, oxo (═O), —COOR¹³, —OR¹³, —C(O)R¹³, —C(O)N(R¹³R^{13a}), —S(O)₂N(R¹³R^{13a}), —S(O)N(R¹³R^{13a}), —S(O)₂R¹³, —S(O)R¹³, —N(R¹³)S(O)₂N(R^{13a}R^{13b}), —SR¹³, —N(R³R^{13a}), —NO₂, —OC(O)R¹³, —N(R¹³)C(O)R^{13a}, —N(R³)S(O)₂R^{13a}, —N(R¹³)S(O)R^{13a}, —N(R¹³)C(O)OR^{13a}, —N(R¹³)C(O)N(R^{13a}R^{13b}), —OC(O)N(R¹³R^{13a}), and C₁₋₆ alkyl; wherein C₁₋₆ alkyl is optionally substituted with one or more halogen, which are the same or different;
each —R¹, —R^{12a}, —R¹³, —R^{13a}, and —R^{13b} is independently selected from the group consisting of-H, and C₁₋₆ alkyl; wherein C₁₋₆ alkyl is optionally substituted with one or more halogen, which are the same or different;
optionally, one or more of the pairs —R¹/—R^{1a}, —R²/—R^{2a}, —R³/—R^{3a}, —R⁶/—R^{6a}, and —R⁷/—R^{7a} are joined together with the atom to which they are attached to form a C₃₋₁₀ cycloalkyl or a 3- to 10-membered heterocyclyl;
optionally, one or more of the pairs —R¹/—R², —R¹/—R³, —R¹/—R⁴, —R¹/—R⁵, —R¹/—R⁶, —R¹/—R⁷, —R²/—R³, —R²/—R⁴, —R²/—R⁵, —R²/—R⁶, —R²/—R⁷, —R³/—R⁴, —R³/—R⁵, —R³/—R⁶, —R³/—R⁷, —R⁴/—R⁵, —R⁴/—R⁶, —R⁴/—R⁷, —R⁵/—R⁶, —R⁵/—R⁷, and —R⁶/—R⁷ are joint together with the atoms to which they are attached to form a ring A;
A is selected from the group consisting of phenyl; naphthyl; indenyl; indanyl; tetralinyl; C₃₋₁₀ cycloalkyl; 3- to 10-membered heterocyclyl; and 8- to 11-membered heterobicyclyl;
wherein -L¹- is substituted with at least one -L²-Z and wherein -L¹- is optionally further substituted.

The optional further substituents of -L¹- of formula (III) are in certain embodiments as described above.

In certain embodiments -L¹- of formula (III) is substituted with one moiety -L²-Z.

In certain embodiments -L¹- of formula (III) is not further substituted.

In certain embodiments -L¹- is as disclosed in EP1536334B1, WO2009/009712A1, WO2008/034122A1, WO2009/143412A2, WO2011/082368A2, and U.S. Pat. No. 8,618,124B2, which are herewith incorporated by reference in their entirety.

In certain embodiments -L¹- is as disclosed in U.S. Pat. No. 8,946,405B2 and U.S. Pat. No. 8,754,190B2, which are herewith incorporated by reference in their entirety. Accordingly, in certain embodiments -L¹- is of formula (IV):

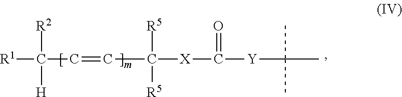

wherein
the dashed line indicates attachment to -D- and wherein attachment is through a functional group of -D- selected from the group consisting of —OH, —SH and —NH₂;
m is 0 or 1;
at least one or both of —R¹ and —R² is/are independently of each other selected from the group consisting of —CN, —NO₂, optionally substituted aryl, optionally substituted heteroaryl, optionally substituted alkenyl, optionally substituted alkynyl, —C(O)R³, —S(O)R³, —S(O)₂R³, and —SR⁴,
one and only one of —R¹ and —R² is selected from the group consisting of —H, optionally substituted alkyl, optionally substituted arylalkyl, and optionally substituted heteroarylalkyl;
—R³ is selected from the group consisting of —H, optionally substituted alkyl, optionally substituted aryl, optionally substituted arylalkyl, optionally substituted heteroaryl, optionally substituted heteroarylalkyl, —OR⁹ and —N(R⁹)₂;
—R⁴ is selected from the group consisting of optionally substituted alkyl, optionally substituted aryl, optionally substituted arylalkyl, optionally substituted heteroaryl, and optionally substituted heteroarylalkyl;
each —R⁵ is independently selected from the group consisting of —H, optionally substituted alkyl, optionally substituted alkenylalkyl, optionally substituted alkynylalkyl, optionally substituted aryl, optionally substituted arylalkyl, optionally substituted heteroaryl and optionally substituted heteroarylalkyl;
—R⁹ is selected from the group consisting of —H and optionally substituted alkyl;
—Y— is absent and —X— is —O— or —S—; or
—Y— is —N(Q)CH₂— and —X— is —O—;
Q is selected from the group consisting of optionally substituted alkyl, optionally substituted aryl, optionally substituted arylalkyl, optionally substituted heteroaryl and optionally substituted heteroarylalkyl;
optionally, —R¹ and —R² may be joined to form a 3 to 8-membered ring; and optionally, both —R⁹ together with the nitrogen to which they are attached form a heterocyclic ring;
wherein -L¹- is substituted with at least one -L²-Z and wherein -L¹- is optionally further substituted.

Only in the context of formula (IV) the terms used have the following meaning:

The term “alkyl” as used herein includes linear, branched or cyclic saturated hydrocarbon groups of 1 to 8 carbons, or in certain embodiments 1 to 6 or 1 to 4 carbon atoms.

The term “alkoxy” includes alkyl groups bonded to oxygen, including methoxy, ethoxy, isopropoxy, cyclopropoxy, cyclobutoxy, and similar.

The term “alkenyl” includes non-aromatic unsaturated hydrocarbons with carbon-carbon double bonds.

The term “alkynyl” includes non-aromatic unsaturated hydrocarbons with carbon-carbon triple bonds.

The term “aryl” includes aromatic hydrocarbon groups of 6 to 18 carbons, such as 6 to 10 carbons, including groups such as phenyl, naphthyl, and anthracenyl. The term “heteroaryl” includes aromatic rings comprising 3 to 15 carbons containing at least one N, O or S atom, such as 3 to 7 carbons containing at least one N, O or S atom, including groups such as pyrrolyl, pyridyl, pyrimidinyl, imidazolyl, oxazolyl, isoxazolyl, thiazolyl, isothiazolyl, quinolyl, indolyl, indenyl, and similar.

In some instance, alkenyl, alkynyl, aryl or heteroaryl moieties may be coupled to the remainder of the molecule through an alkylene linkage. Under those circumstances, the substituent will be referred to as alkenylalkyl, alkynylalkyl, arylalkyl or heteroarylalkyl, indicating that an alkylene moiety is between the alkenyl, alkynyl, aryl or heteroaryl moiety and the molecule to which the alkenyl, alkynyl, aryl or heteroaryl is coupled.

The term “halogen” includes bromo, fluoro, chloro and iodo.

The term “heterocyclic ring” refers to a 4 to 8 membered aromatic or non-aromatic ring comprising 3 to 7 carbon atoms and at least one N, O, or S atom. Examples are piperidinyl, piperazinyl, tetrahydropyranyl, pyrrolidine, and tetrahydrofuranyl, as well as the exemplary groups provided for the term “heteroaryl” above.

When a ring system is optionally substituted, suitable substituents are selected from the group consisting of alkyl, alkenyl, alkynyl, or an additional ring, each optionally further substituted.

Optional substituents on any group, including the above, include halo, nitro, cyano, —OR, —SR, —NR₂, —OCOR, —NRCOR, —COOR, —CONR₂, —SOR, —SO₂R, —SONR₂, —SO₂N R₂, wherein each R is independently alkyl, alkenyl, alkynyl, aryl or heteroaryl, or two R groups taken together with the atoms to which they are attached form a ring.

In certain embodiments -L¹- of formula (IV) is substituted with one moiety -L²-Z.

In certain embodiments -L¹- of formula (IV) is not further substituted.

In certain embodiments -L¹- is as disclosed in WO2013/036857A1, which is herewith incorporated by reference in its entirety. Accordingly, in certain embodiments -L¹- is of formula (V):

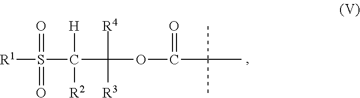

wherein
the dashed line indicates attachment to -D- through an amine functional group of -D-;
—R¹ is selected from the group consisting of optionally substituted C₁-C₆ linear, branched, or cyclic alkyl; optionally substituted aryl; optionally substituted heteroaryl; alkoxy; and —NR⁵₂;
—R² is selected from the group consisting of —H; optionally substituted C₁-C₆ alkyl; optionally substituted aryl; and optionally substituted heteroaryl;
—R³ is selected from the group consisting of —H; optionally substituted C₁-C₆ alkyl; optionally substituted aryl; and optionally substituted heteroaryl;
—R⁴ is selected from the group consisting of —H; optionally substituted C₁-C₆ alkyl; optionally substituted aryl; and optionally substituted heteroaryl;
each —R⁵ is independently of each other selected from the group consisting of —H; optionally substituted C₁-C₆ alkyl; optionally substituted aryl; and optionally substituted heteroaryl; or when taken together two —R⁵ can be cycloalkyl or cycloheteroalkyl;
wherein -L¹- is substituted with at least one -L²-Z and wherein -L¹- is optionally further substituted.

Only in the context of formula (V) the terms used have the following meaning:

“Alkyl”, “alkenyl”, and “alkynyl” include linear, branched or cyclic hydrocarbon groups of 1-8 carbons or 1-6 carbons or 1-4 carbons wherein alkyl is a saturated hydrocarbon, alkenyl includes one or more carbon-carbon double bonds and alkynyl includes one or more carbon-carbon triple bonds. Unless otherwise specified these contain 1-6 C.

“Aryl” includes aromatic hydrocarbon groups of 6-18 carbons, such as 6-10 carbons, including groups such as phenyl, naphthyl, and anthracene “Heteroaryl” includes aromatic rings comprising 3-15 carbons containing at least one N, O or S atom, such as 3-7 carbons containing at least one N, O or S atom, including groups such as pyrrolyl, pyridyl, pyrimidinyl, imidazolyl, oxazolyl, isoxazolyl, thiszolyl, isothiazolyl, quinolyl, indolyl, indenyl, and similar.

The term “substituted” means an alkyl, alkenyl, alkynyl, aryl, or heteroaryl group comprising one or more substituent groups in place of one or more hydrogen atoms. Substituents may generally be selected from halogen including F, Cl, Br, and I; lower alkyl including linear, branched, and cyclic; lower haloalkyl including fluoroalkyl, chloroalkyl, bromoalkyl, and iodoalkyl; OH; lower alkoxy including linear, branched, and cyclic; SH; lower alkylthio including linear, branched and cyclic; amino, alkylamino, dialkylamino, silyl including alkylsilyl, alkoxysilyl, and arylsilyl; nitro; cyano; carbonyl; carboxylic acid, carboxylic ester, carboxylic amide, aminocarbonyl; aminoacyl; carbamate; urea; thiocarbamate; thiourea; ketone; sulfone; sulfonamide; aryl including phenyl, naphthyl, and anthracenyl; heteroaryl including 5-member heteroaryls including as pyrrole, imidazole, furan, thiophene, oxazole, thiazole, isoxazole, isothiazole, thiadiazole, triazole, oxadiazole, and tetrazole, 6-member heteroaryls including pyridine, pyrimidine, pyrazine, and fused heteroaryls including benzofuran, benzothiophene, benzoxazole, benzimidazole, indole, benzothiazole, benzisoxazole, and benzisothiazole.

In certain embodiments -L¹- of formula (V) is substituted with one moiety -L²-Z.

In certain embodiments -L¹- of formula (V) is not further substituted.

In certain embodiments -L¹- is as disclosed in U.S. Pat. No. 7,585,837B2, which is herewith incorporated by reference in its entirety. Accordingly, in certain embodiments -L¹- is of formula (VI):

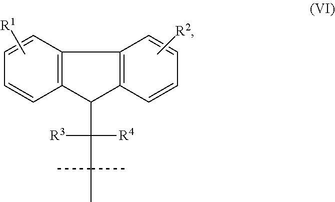

wherein
the dashed line indicates attachment to -D through an amine functional group of -D; R¹ and R² are independently selected from the group consisting of hydrogen, alkyl, alkoxy, alkoxyalkyl, aryl, alkaryl, aralkyl, halogen, nitro, —SO₃H, —SO₂NHR⁵, amino, ammonium, carboxyl, PO₃H₂, and OPO₃H₂;
R³, R⁴, and R⁵ are independently selected from the group consisting of hydrogen, alkyl, and aryl;
wherein -L¹- is substituted with at least one -L²-Z and wherein -L¹- is optionally further substituted.

Suitable substituents for formulas (VI) are alkyl (such as C₁₋₆ alkyl), alkenyl (such as C₂₋₆ alkenyl), alkynyl (such as C₂₋₆ alkynyl), aryl (such as phenyl), heteroalkyl, heteroalkenyl, heteroalkynyl, heteroaryl (such as aromatic 4 to 7 membered heterocycle) or halogen moieties.

Only in the context of formula (VI) the terms used have the following meaning:

The terms “alkyl”, “alkoxy”, “alkoxyalkyl”, “aryl”, “alkaryl” and “aralkyl” mean alkyl radicals of 1-8, such as 1-4 carbon atoms, e.g. methyl, ethyl, propyl, isopropyl and butyl, and aryl radicals of 6-10 carbon atoms, e.g. phenyl and naphthyl. The term “halogen” includes bromo, fluoro, chloro and iodo.

In certain embodiments -L¹- of formula (VI) is substituted with one moiety -L²-Z.

In certain embodiments -L¹- of formula (VI) is not further substituted.

A further preferred embodiment for -L¹- is disclosed in WO2002/089789A1, which is herewith incorporated by reference in its entirety. Accordingly, a preferred moiety -L¹- is of formula (VII):

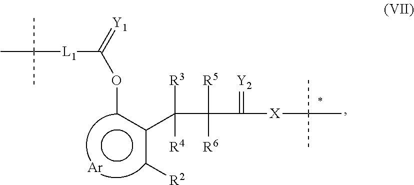

wherein
the dashed line indicates attachment to -D- through an amine functional group of -D-;
L₁ is a bifunctional linking group,
Y₁ and Y₂ are independently O, S or NR⁷; R², R³, R⁴, R⁵, R⁶ and R⁷ are independently selected from the group consisting of hydrogen, C₁₋₆ alkyls, C₃₋₁₂ branched alkyls, C₃₋₈ cycloalkyls, C₁₋₆ substituted alkyls, C_{3.8} substituted cycloalkyls, aryls, substituted aryls, aralkyls, C₁₋₆ heteroalkyls, substituted C₁.6 heteroalkyls, C₁₋₆ alkoxy, phenoxy, and C₁₋₆ heteroalkoxy;
Ar is a moiety which when included in formula (VII) forms a multisubstituted aromatic hydrocarbon or a multi-substituted heterocyclic group;
X is a chemical bond or a moiety that is actively transported into a target cell, a hydrophobic moiety, or a combination thereof,
y is 0 or 1;
wherein -L¹- is substituted with at least one -L²-Z and wherein -L¹- is optionally further substituted.

Only in the context of formula (VII) the terms used have the following meaning:

The term “alkyl” shall be understood to include, e.g., straight, branched, substituted C₁₋₁₂ alkyls, including alkoxy, C₃₋₈ cycloalkyls or substituted cycloalkyls, etc.

The term “substituted” shall be understood to include adding or replacing one or more atoms contained within a functional group or compounds with one or more different atoms.

Substituted alkyls include carboxyalkyls, aminoalkyls, dialkylaminos, hydroxyalkyls and mercaptoalkyls; substituted cycloalkyls include moieties such as 4-chlorocyclohexyl; aryls include moieties such as napthyl; substituted aryls include moieties such as 3-bromo-phenyl; aralkyls include moieties such as toluyl; heteroalkyls include moieties such as ethylthiophene; substituted heteroalkyls include moieties such as 3-methoxythiophone; alkoxy includes moeities such as methoxy; and phenoxy includes moieties such as 3-nitrophenoxy. Halo- shall be understood to include fluoro, chloro, iodo and bromo.

In certain embodiments -L¹- of formula (VII) is substituted with one moiety -L²-Z.

In certain embodiments -L¹- of formula (VII) is not further substituted.

In certain embodiments -L¹- comprises a substructure of formula (VIII)

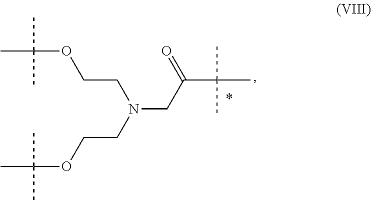

wherein
the dashed line marked with the asterisk indicates attachment to a nitrogen of -D- through an amide bond;
the unmarked dashed lines indicate attachment to the remainder of -L¹-; and
wherein -L¹- is substituted with at least one -L²-Z and wherein -L¹- is optionally further substituted.

In certain embodiments -L¹- of formula (VIII) is substituted with one moiety -L²-Z.

In certain embodiments -L¹- of formula (VIII) is not further substituted.

In certain embodiments -L¹- comprises a substructure of formula (IX)

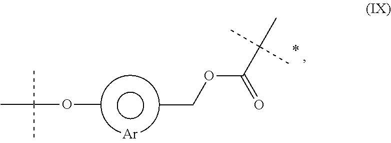

wherein
the dashed line marked with the asterisk indicates attachment to a nitrogen of -D- through a carbamate bond;
the unmarked dashed lines indicate attachment to the remainder of -L¹-; and
wherein -L¹- is substituted with at least one -L²-Z and wherein -L¹- is optionally further substituted.

In certain embodiments -L¹- of formula (IX) is substituted with one moiety -L²-Z.

In certain embodiments -L¹- of formula (IX) is not further substituted.

In certain embodiments -L¹- has a structure as disclosed in WO2020/206358 A1. Accordingly, in certain embodiments the moiety -L¹- is of formula (X):

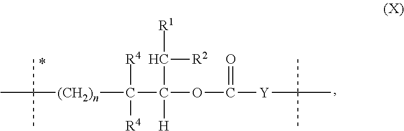

wherein
the unmarked dashed line indicates attachment to -D-;
the dashed line marked with the asterisk indicates attachment to -L²-Z;
n is an integer selected from the group consisting of 0, 1, 2, 3, 4, 5 and 6;
—R¹ and —R² are independently an electron-withdrawing group, alkyl, or —H, and wherein at least one of —R¹ or —R² is an electron-withdrawing group;
each —R⁴ is independently C₁-C₃ alkyl or the two —R⁴ are taken together with the carbon atom to which they are attached to form a 3- to 6-membered ring; and
—Y— is absent when -D- is a drug moiety connected through an amine, or —Y— is —N(R⁶)CH₂— when -D- is a drug moiety connected through a phenol, alcohol, thiol, thiophenol, imidazole, or non-basic amine; wherein —R⁶ is optionally substituted C₁-C₆ alkyl, optionally substituted aryl, or optionally substituted heteroaryl.

In certain embodiments n of formula (X) is an integer selected from 1, 2, 3, 4, 5 and 6. In certain embodiments n of formula (X) is an integer selected from 1, 2 and 3. In certain embodiments n of formula (X) is an integer from 0, 1, 2 and 3. In certain embodiments n of formula (X) is 1. In certain embodiments n of formula (X) is 2. In certain embodiments n of formula (X) is 3.

In certain embodiments the electron-withdrawing group of —R¹ and —R² of formula (X) is selected from the group consisting of —CN; —NO₂; optionally substituted aryl; optionally substituted heteroaryl; optionally substituted alkenyl; optionally substituted alkynyl; —COR³, —SOR³, or —SO₂R³, wherein —R³ is —H, optionally substituted alkyl, optionally substituted aryl, optionally substituted arylalkyl, optionally substituted heteroaryl, optionally substituted heteroarylalkyl, —OR⁸ or —NR⁸₂, wherein each —R⁸ is independently —H or optionally substituted alkyl, or both —R⁸ groups are taken together with the nitrogen to which they are attached to form a heterocyclic ring; or —SR⁹, wherein —R⁹ is optionally substituted alkyl, optionally substituted aryl, optionally substituted arylalkyl, optionally substituted heteroaryl, or optionally substituted heteroarylalkyl.

In certain embodiments the electron-withdrawing group of —R¹ and —R² of formula (X) is —CN. In certain embodiments the electron-withdrawing group of —R¹ and —R² of formula (X) is —NO₂. In certain embodiments the electron-withdrawing group of —R¹ and —R² of formula (X) is optionally substituted aryl comprising 6 to 10 carbons. In certain embodiments the electron-withdrawing group of —R¹ and —R² of formula (X) is optionally substituted phenyl, naphthyl, or anthracenyl. In certain embodiments the electron-withdrawing group of —R¹ and —R² of formula (X) is optionally substituted heteroaryl comprising 3 to 7 carbons and comprising at least one N, O, or S atom. In certain embodiments the electron-withdrawing group of —R¹ and —R² of formula (X) is optionally substituted pyrrolyl, pyridyl, pyrimidinyl, imidazolyl, oxazolyl, isoxazolyl, thiazolyl, isothiazolyl, quinolyl, indolyl, or indenyl. In certain embodiments the electron-withdrawing group of —R¹ and —R² of formula (X) is optionally substituted alkenyl containing 2 to 20 carbon atoms. In certain embodiments the electron-withdrawing group of —R¹ and —R² of formula (X) is optionally substituted alkynyl comprising 2 to 20 carbon atoms. In certain embodiments the electron-withdrawing group of —R¹ and —R² of formula (X) is —COR³, —SOR³, or —SO₂R³, wherein —R³ is —H, optionally substituted alkyl comprising 1 to 20 carbon atoms, optionally substituted aryl, optionally substituted arylalkyl, optionally substituted heteroaryl, optionally substituted heteroarylalkyl, —OR⁸ or —NR⁸₂, wherein each —R⁸ is independently —H or optionally substituted alkyl comprising 1 to 20 carbon atoms, or both —R⁸ groups are taken together with the nitrogen to which they are attached to form a heterocyclic ring. In certain embodiments the electron-withdrawing group of —R¹ and —R² of formula (X) is —SR⁹, wherein —R⁹ is optionally substituted alkyl comprising 1 to 20 carbon atoms, optionally substituted aryl, optionally substituted arylalkyl, optionally substituted heteroaryl, or optionally substituted heteroarylalkyl.

In certain embodiments at least one of —R¹ or —R² of formula (X) is —CN, —SOR³ or —SO₂R³. In certain embodiments at least one of —R¹ and —R² of formula (X) is —CN or —SO₂R³. In certain embodiments at least one of —R¹ and —R² of formula (X) is —CN or —SO₂R³, wherein —R³ is optionally substituted alkyl, optionally substituted aryl, or —NR⁸₂. In certain embodiments at least one of —R¹ and —R² of formula (X) is —CN, —SO₂N(CH₃)₂, —SO₂CH₃, phenyl substituted with —SO₂, phenyl substituted with —SO₂ and —Cl, —SO₂N(CH₂CH₂)₂O, —SO₂CH(CH₃)₂, —SO₂N(CH₃)(CH₂CH₃), or —SO₂N(CH₂CH₂OCH₃)₂.

In certain embodiments each —R⁴ of formula (X) is independently C₁-C₃ alkyl. In certain embodiments both —R⁴ are methyl.

In certain embodiments —Y— of formula (X) is absent. In certain embodiments —Y— of formula (X) is —N(R⁶)CH₂—.

In certain embodiments -L¹- is of formula (X), wherein n is 1, —R¹ is —CN, —R² is —H, and —R⁴ is —CH₃. In certain embodiments -L¹- is of formula (X), wherein n is 1, —R¹ is —SO₂N(CH₃)₂, —R² is —H, and —R⁴ is —CH₃. In certain embodiments -L¹- is of formula (X), wherein n is 1, —R¹ is SO₂CH₃, —R² is —H, and —R⁴ is —CH₃. In certain embodiments -L¹- is of formula (X), wherein n is 1, —R¹ is —SO₂N(CH₂CH₂)₂CHCH₃, —R² is —H, and —R⁴ is —CH₃. In certain embodiments -L¹- is of formula (X), wherein n is 1, —R¹ is phenyl substituted with —SO₂, —R² is —H, and —R⁴ is —CH₃. In certain embodiments -L¹- is of formula (X), wherein n is 1, —R¹ is phenyl substituted with —SO₂ and —Cl, —R² is —H, and —R⁴ is —CH₃. In certain embodiments -L¹- is of formula (X), wherein n is 1, —R¹ is —SO₂N(CH₂CH₂)₂O, —R² is —H, and —R⁴ is —CH₃. In certain embodiments -L¹- is of formula (X), wherein n is 1, —R¹ is —SO₂CH(CH₃)₂, —R² is —H, and —R⁴ is —CH₃. In certain embodiments -L¹- is of formula (X), wherein n is 1, —R¹ is —SO₂N(CH₃)(CH₂CH₃), —R² is —H, and —R⁴ is —CH₃. In certain embodiments -L¹- is of formula (X), wherein n is 1, —R¹ is —SO₂N(CH₂CH₂OCH₃)₂, —R² is —H, and —R⁴ is —CH₃. In certain embodiments -L¹- is of formula (X), wherein n is 1, —R¹ is phenyl substituted with-SO₂ and —CH₃, —R² is —H, and —R⁴ is —CH₃.

In certain embodiments -L¹- is of formula (X), wherein n is 2, —R¹ is —CN, —R² is —H, and —R⁴ is —CH₃. In certain embodiments -L¹- is of formula (X), wherein n is 2, —R¹ is —SO₂N(CH₃)₂, —R² is —H, and —R⁴ is —CH₃. In certain embodiments -L¹- is of formula (X), wherein n is 2, —R¹ is SO₂CH₃, —R² is —H, and —R⁴ is —CH₃. In certain embodiments -L¹- is of formula (X), wherein n is 2, —R¹ is —SO₂N(CH₂CH₂)₂CHCH₃, —R² is —H, and —R⁴ is —CH₃. In certain embodiments -L¹- is of formula (X), wherein n is 2, —R¹ is phenyl substituted with —SO₂, —R² is —H, and —R⁴ is —CH₃.

In certain embodiments -L¹- is of formula (X), wherein n is 2, —R¹ is phenyl substituted with —SO₂ and —Cl, —R² is —H, and —R⁴ is —CH₃. In certain embodiments -L¹- is of formula (X), wherein n is 2, —R¹ is —SO₂N(CH₂CH₂)₂O, —R² is —H, and —R⁴ is —CH₃. In certain embodiments -L¹- is of formula (X), wherein n is 2, —R¹ is —SO₂CH(CH₃)₂, —R² is —H, and —R⁴ is —CH₃. In certain embodiments -L¹- is of formula (X), wherein n is 2, —R¹ is —SO₂N(CH₃)(CH₂CH₃), —R² is —H, and —R⁴ is —CH₃. In certain embodiments -L¹- is of formula (X), wherein n is 2, —R¹ is —SO₂N(CH₂CH₂OCH₃)₂, —R² is —H, and —R⁴ is —CH₃. In certain embodiments -L¹- is of formula (X), wherein n is 2, —R¹ is phenyl substituted with —SO₂ and —CH₃, —R² is —H, and —R⁴ is —CH₃.

In certain embodiments -L¹- is of formula (X), wherein n is 3, —R¹ is —CN, —R² is —H, and —R⁴ is —CH₃. In certain embodiments -L¹- is of formula (X), wherein n is 3, —R¹ is —SO₂N(CH₃)₂, —R² is —H, and —R⁴ is —CH₃. In certain embodiments -L¹- is of formula (X), wherein n is 3, —R¹ is SO₂CH₃, —R² is —H, and —R⁴ is —CH₃. In certain embodiments -L¹- is of formula (X), wherein n is 3, —R¹ is —SO₂N(CH₂CH₂)₂CHCH₃, —R² is —H, and —R⁴ is —CH₃. In certain embodiments -L¹- is of formula (X), wherein n is 3, —R¹ is phenyl substituted with —SO₂, —R² is —H, and —R⁴ is —CH₃. In certain embodiments -L¹- is of formula (X), wherein n is 3, —R¹ is phenyl substituted with —SO₂ and —Cl, —R² is —H, and —R⁴ is —CH₃. In certain embodiments -L¹- is of formula (X), wherein n is 3, —R¹ is —SO₂N(CH₂CH₂)₂O, —R² is —H, and —R⁴ is —CH₃. In certain embodiments -L¹- is of formula (X), wherein n is 3, —R¹ is —SO₂CH(CH₃)₂, —R² is —H, and —R⁴ is —CH₃. In certain embodiments -L¹- is of formula (X), wherein n is 3, —R¹ is —SO₂N(CH₃)(CH₂CH₃), —R² is —H, and —R⁴ is —CH₃. In certain embodiments -L¹- is of formula (X), wherein n is 3, —R¹ is —SO₂N(CH₂CH₂OCH₃)₂, —R² is —H, and —R⁴ is —CH₃. In certain embodiments -L¹- is of formula (X), wherein n is 3, —R¹ is phenyl substituted with —SO₂ and —CH₃, —R² is —H, and —R⁴ is —CH₃.

Only in the context of formula (X) the terms used have the following meaning:

The term “alkyl” refers to linear, branched, or cyclic saturated hydrocarbon groups of 1 to 20, 1 to 12, 1 to 8, 1 to 6, or 1 to 4 carbon atoms. In certain embodiments an alkyl is linear or branched. Examples of linear or branched alkyl groups include methyl, ethyl, n-propyl, isopropyl, n-butyl, t-butyl, isobutyl, sec-butyl, n-pentyl, n-hexyl, n-heptyl, n- octyl, n-nonyl, and n-decyl. In certain embodiments an alkyl is cyclic. Examples of cyclic alkyl groups include cyclopropyl, cyclobutyl, cyclopentyl, cyclopentadienyl, and cyclohexyl.

The term “alkoxy” refers to alkyl groups bonded to oxygen, including methoxy, ethoxy, isopropoxy, cyclopropoxy, and cyclobutoxy.

The term “alkenyl” refers to non-aromatic unsaturated hydrocarbons with carbon-carbon double bonds and 2 to 20, 2 to 12, 2 to 8, 2 to 6, or 2 to 4 carbon atoms.

The term “alkynyl” refers to non-aromatic unsaturated hydrocarbons with carbon-carbon triple bonds and 2 to 20, 2 to 12, 2 to 8, 2 to 6, or 2 to 4 carbon atoms.

The term “aryl” refers to aromatic hydrocarbon groups of 6 to 18 carbons, preferably 6 to 10 carbons, including groups such as phenyl, naphthyl, and anthracenyl. The term “heteroaryl” refers to aromatic rings comprising 3 to 15 carbons comprising at least one N, O or S atom, preferably 3 to 7 carbons comprising at least one N, O or S atom, including groups such as pyrrolyl, pyridyl, pyrimidinyl, imidazolyl, oxazolyl, isoxazolyl, thiazolyl, isothiazolyl, quinolyl, indolyl, and indenyl.

In certain embodiments alkenyl, alkynyl, aryl or heteroaryl moieties may be coupled to the remainder of the molecule through an alkyl linkage. Under those circumstances, the substituent will be referred to as alkenylalkyl, alkynylalkyl, arylalkyl or heteroarylalkyl, indicating that an alkylene moiety is between the alkenyl, alkynyl, aryl or heteroaryl moiety and the molecule to which the alkenyl, alkynyl, aryl or heteroaryl is coupled.

The term “halogen” or “halo” refers to bromo, fluoro, chloro and iodo.

The term “heterocyclic ring” or “heterocyclyl” refers to a 3- to 15-membered aromatic or non-aromatic ring comprising at least one N, O, or S atom. Examples include piperidinyl, piperazinyl, tetrahydropyranyl, pyrrolidine, and tetrahydrofuranyl, as well as the exemplary groups provided for the term “heteroaryl” above. In certain embodiments a heterocyclic ring or heterocyclyl is non-aromatic. In certain embodiments a heterocyclic ring or heterocyclyl is aromatic.

The term “optionally substituted” refers to a group may be unsubstituted or substituted by one or more (e.g., 1, 2, 3, 4 or 5) of the substituents which may be the same or different. Examples of substituents include alkyl, alkenyl, alkynyl, halogen, —CN, —OR^{aa}, —SR^{aa}, —NR^{aa}R^{bb}, —NO₂, —C═NH(OR^{aa}), —C(O)R^{aa}, —OC(O)R^{aa}, —C(O)OR^{aa}, —C(O)NR^{aa}R^{bb}, —OC(O)NR^{aa}R^{bb}, —NR^{aa}C(O)R^{bb}, —NR^{aa}C(O)OR^{bb}, —S(O)R^{aa}, —S(O)₂R^{aa}, —NR^{aa}S(O)R^{bb}, —C(O)NR^{aa}S(O)R^{bb}, —NR^{aa}S(O)₂R^{bb}, —C(O)NR^{aa}S(O)₂R^{bb}, —S(O)NR^{aa}R^{bb}, —S(O)₂NR^{aa}R^{bb}, —P(O)(OR^{aa})(OR^{bb}), heterocyclyl, heteroaryl, or aryl, wherein the alkyl, alkenyl, alkynyl, cycloalkyl, heterocyclyl, heteroaryl, and aryl are each independently optionally substituted by —R^{cc}, wherein —R^{aa} and —R^{bb} are each independently —H, alkyl, alkenyl, alkynyl, heterocyclyl, heteroaryl, or aryl, or —R^{aa} and —R^{bb} are taken together with the nitrogen atom to which they attach to form a heterocyclyl, which is optionally substituted by alkyl, alkenyl, alkynyl, halogen, hydroxyl, alkoxy, or —CN, and wherein: each —R^{cc} is independently alkyl, alkenyl, alkynyl, halogen, heterocyclyl, heteroaryl, aryl, —CN, or —NO₂.

In certain embodiments -L¹- has a structure as disclosed in formula I of WO2021/242756 A1. Accordingly, in certain embodiments the moiety -L¹- is of formula (XIIa):

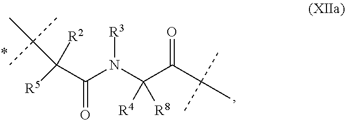

wherein
the unmarked dashed line indicates attachment to -D-;
the dashed line marked with the asterisk indicates attachment to -L²-;
—R², —R⁴ and —R⁸ are independently selected from the group consisting of —H or C₁₋₄ alkyl;
—R³ is C₁₋₄ alkyl or —R³ and —R⁴ together with the atoms to which they are attached form a 5- or 6-membered heterocyclic ring;
—R⁵ is —NH₂;
with the proviso that when —R⁴ and —R³ together with the atoms to which they are attached from a 5- or 6-membered heterocyclic ring —R² is not —H; and wherein the -L¹- of formula (XIIa) is optionally substituted.

In certain embodiments both —R⁴ and —R⁸ of formula (XIIa) are —H.

In certain embodiments —R³ is methyl. In certain embodiments —R³ is —H.

In certain embodiments —R² is —H.

In certain embodiments -L¹- is of formula (XIIa-i)

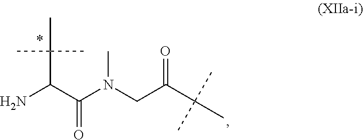

the unmarked dashed line indicates attachment to -D-; and
the dashed line marked with the asterisk indicates attachment to -L²-.

In certain embodiments -L¹- is of formula (XIIa-ii)

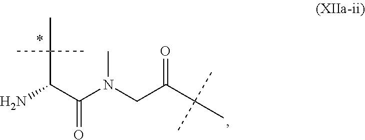

the unmarked dashed line indicates attachment to -D-; and
the dashed line marked with the asterisk indicates attachment to -L²-.

In certain embodiments -L¹- is of formula (XIIa-iii)

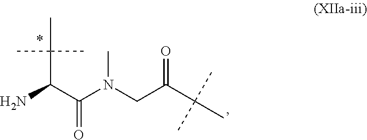

the unmarked dashed line indicates attachment to -D-; and
the dashed line marked with the asterisk indicates attachment to -L²-.

In certain embodiments -L¹- has a structure as disclosed in formula II of WO2022/096636 A1. Accordingly, in certain embodiments the moiety -L¹- is of formula (XIIb):

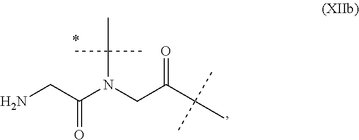

wherein
the unmarked dashed line indicates attachment to -D-; and
the dashed line marked with the asterisk indicates attachment to -L²-.

In certain embodiments -L²- is absent.

In certain embodiments -L²- is a spacer moiety, in particular selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2} which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR³)(R^{y3a})—, —N(R^{y3})C(O)N(R^{y3a})—, and —OC(O)N(R^{y3})—;
—R^{y1} and —R^{y1a} are independently of each other selected from the group consisting of —H, -T, C₁₋₅₀ alkyl, C₂₋₅₀ alkenyl, and C₂₋₅₀ alkynyl; wherein -T, C₁₋₅₀ alkyl, C₂₋₅₀ alkenyl, and C₂₋₅₀ alkynyl are optionally substituted with one or more —R^{y2}, which are the same or different, and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR⁴)(R^{y4a})—, —N(R^{y4})C(O)N(R^{y4a})—, and —OC(O)N(R^{y4})—;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;
each —R^{y2} is independently selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and
each —R^{y3}, —R^{y3a}, —R^{y4}, —R^{y4a}, —R^{y5}, —R^{y5a} and —R^{y5b} is independently selected from the group consisting of —H, and C₁₋₆ alkyl, wherein C₁₋₆ alkyl is optionally substituted with one or more halogen, which are the same or different.

In certain embodiments -L²- is selected from -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₂₀ alkyl, C₂₋₂₀ alkenyl, and C₂₋₂₀ alkynyl are optionally substituted with one or more —R^{y2}, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR³)(R^{y3a})—, —N(R^{y3})C(O)N(R^{y3a})—, and —OC(O)N(R^{y3})—;
—R^{y1} and —R^{y1a} are independently of each other selected from the group consisting of —H, -T, C₁₋₁₀ alkyl, C₂₋₁₀ alkenyl, and C₂₋₁₀ alkynyl; wherein -T, C₁₋₁₀ alkyl, C₂₋₁₀ alkenyl, and C₂₋₁₀ alkynyl are optionally substituted with one or more —R^{y2}, which are the same or different, and wherein C₁₋₁₀ alkyl, C₂₋₁₀ alkenyl, and C₂₋₁₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR⁴)(R^{y4a})—, —N(R^{y4})C(O)N(R^{y4a})—, and —OC(O)N(R^{y4})—;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;
—R^{y2} is selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5}) C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and
each —R^{y3}, —R^{y3a}, —R^{y4}, —R^{y4a}, —R^{y5}, —R^{y5a} and —R^{y5b} is independently of each other selected from the group consisting of —H, and C₁₋₆ alkyl; wherein C₁₋₆ alkyl is optionally substituted with one or more halogen, which are the same or different.

In certain embodiments -L²- is selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;
—R^{y1} and —R^{y1a} are independently selected from the group consisting of —H, -T, C₁₋₁₀ alkyl, C₂₋₁₀ alkenyl, and C₂₋₁₀ alkynyl;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl;
each —R^{y2} is independently selected from the group consisting of halogen, and C₁₋₆ alkyl; and
each —R^{y3}, —R^{y3a}, —R^{y4}, —R^{y4a}, —R^{y5}, —R^{y5a} and —R^{y5b} is independently of each other selected from the group consisting of —H, and C₁₋₆ alkyl; wherein C₁₋₆ alkyl is optionally substituted with one or more halogen, which are the same or different.

In certain embodiments -L²- is a C₁₋₂₀ alkyl chain, which is optionally interrupted by one or more groups independently selected from —O—, —S—, -T- and —C(O)N(R^{y1})—; and which C₁₋₂₀ alkyl chain is optionally substituted with one or more groups independently selected from —OH, -T and —C(O)N(R^{y6}R^{y6a}); wherein —R^{y1}, —R^{y6}, —R^{y6a} are independently selected from the group consisting of H and C₁₋₄ alkyl and wherein T is selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl.

In certain embodiments -L²- has a molecular weight in the range of from 14 g/mol to 750 g/mol.

In certain embodiments -L²- comprises a moiety selected from

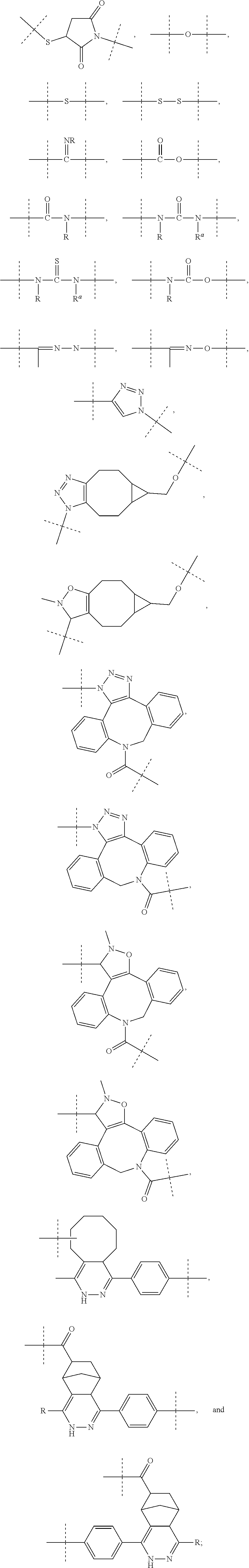

wherein
—R and —R^{a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

In certain embodiments -L²- has a chain length of 1 to 20 atoms. In certain embodiments -L²- has a chain length of 2 to 10 atoms.

In certain embodiments -L²- has a chain length of at least 35 atoms. In certain embodiments -L²- has a chain length of at least 50 atoms. In certain embodiments -L²- has a chain length of at least 75 atoms. In certain embodiments -L²- has a chain length of at least 100 atoms. In certain embodiments -L²- has a chain length of at least 150 atoms. In certain embodiments -L²- has a chain length of at least 200 atoms. In certain embodiments -L²- has a chain length of at least 250 atoms. In certain embodiments -L²- has a chain length of at least 300 atoms. In certain embodiments -L²- has a chain length of at most 3000 atoms. In certain embodiments -L²- has a chain length of at most 2500 atoms. In certain embodiments -L²- has a chain length of at most 2000 atoms. In certain embodiments -L²- has a chain length of at most 1500 atoms. In certain embodiments -L²- has a chain length ranging from 35 to 3000 atoms. In certain embodiments -L²- has a chain length ranging from 50 to 2500 atoms. In certain embodiments -L²- has a chain length ranging from 75 to 2000 atoms. In certain embodiments -L²- has a chain length ranging from 100 to 1500 atoms. In certain embodiments -L²- has a chain length ranging from 125 to 1000 atoms.

In certain embodiments -L²- is of formula (XI)

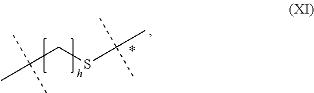

wherein
the unmarked dashed line indicates attachment to -L¹-;
the dashed line marked with the asterisk indicates attachment to the at least one polymeric moiety;
h is an integer selected from the group consisting of 1, 2, 3, 4, 5, 6, 7, 8, 9, 10, 11, 12, 13. 14, 15 and 16.

In certain embodiments -L²- is of formula (XI) and h is 1. In certain embodiments -L²- is of formula (XI) and h is 2. In certain embodiments -L²- is of formula (XI) and h is 3. In certain embodiments -L²- is of formula (XI) and h is 4. In certain embodiments -L²- is of formula (XI) and h is 5. In certain embodiments -L²- is of formula (XI) and h is 6. In certain embodiments -L²- is of formula (XI) and h is 7. In certain embodiments -L²- is of formula (XI) and h is 8. In certain embodiments -L²- is of formula (XI) and h is 9. In certain embodiments -L²- is of formula (XI) and h is 10. In certain embodiments -L²- is of formula (XI) and h is 11. In certain embodiments -L²- is of formula (XI) and h is 12. In certain embodiments -L²- is of formula (XI) and h is 13. In certain embodiments -L²- is of formula (XI) and h is 14. In certain embodiments -L²- is of formula (XI) and h is 15.

In certain embodiments -L²- has a molecular weight of at least 450 Da. In certain embodiments -L²- has a molecular weight of at least 1 kDa. In certain embodiments -L²- has a molecular weight of at least 1.5 kDa. In certain embodiments -L²- has a molecular weight of at least 2 kDa. In certain embodiments -L²- has a molecular weight of at least 2.5 kDa. In certain embodiments -L²- has a molecular weight of at least 3 kDa. In certain embodiments -L²- has a molecular weight of at least 3.5 kDa. In certain embodiments -L²- has a molecular weight of at least 4 kDa. In certain embodiments -L²- has a molecular weight of at least 5 kDa. In certain embodiments -L²- has a maximum molecular weight of 160 kDa. In certain embodiments -L²- has a maximum molecular weight of 120 kDa. In certain embodiments -L²- has a maximum molecular weight of 100 kDa. In certain embodiments -L²- has a maximum molecular weight of 80 kDa. In certain embodiments -L²- has a maximum molecular weight of 70 kDa. In certain embodiments -L²- has a maximum molecular weight of 60 kDa. In certain embodiments -L²- has a maximum molecular weight of 50 kDa. In certain embodiments -L²- has a maximum molecular weight of 40 kDa. In certain embodiments -L²- has a molecular weight of about 450 Da. In certain embodiments -L²- has a molecular weight of about 1 kDa. In certain embodiments -L²- has a molecular weight of about 1.5 kDa. In certain embodiments -L²- has a molecular weight of about 2 kDa. In certain embodiments -L²- has a molecular weight of about 2.5 kDa. In certain embodiments -L²- has a molecular weight of about 3 kDa. In certain embodiments -L²- has a molecular weight of about 3.5 kDa. In certain embodiments -L²- has a molecular weight of about 4 kDa. In certain embodiments -L²- has a molecular weight of about 4.5 kDa. In certain embodiments -L²- has a molecular weight of about 5 kDa. In certain embodiments -L²- has a molecular weight of about 5.5 kDa. In certain embodiments -L²- has a molecular weight of about 6 kDa. In certain embodiments -L²- has a molecular weight of about 6.5 kDa. In certain embodiments -L²- has a molecular weight of about 7 kDa. In certain embodiments -L²- has a molecular weight of about 7.5 kDa. In certain embodiments -L²- has a molecular weight of about 8 kDa. In certain embodiments -L²- has a molecular weight of about 8.5 kDa. In certain embodiments -L²- has a molecular weight of about 9 kDa. In certain embodiments -L²- has a molecular weight of about 9.5 kDa. In certain embodiments -L²- has a molecular weight of about 10 kDa.

In certain embodiments -L²- comprises a polymeric moiety, meaning that it comprises at least one polymer moiety. It is understood that if -L²- comprises one polymer moiety the minimum and maximum molecular weights provided above apply to this one polymer moiety and if -L²- comprises more than one polymer moiety the minimum and maximum molecular weights provided above refer to the minimum and maximum molecular weight of all polymer moieties together.

In certain embodiments the one or more polymer moiety of -L²- has a Flory radius of at least 1.2 nm. In certain embodiments the one or more polymer moiety of -L²- has a Flory radius of at least 1.5 nm. In certain embodiments the one or more polymer moiety of -L²- has a Flory radius of at least 2 nm. In certain embodiments the one or more polymer moiety of -L²- has a Flory radius of at least 2.5 nm. In certain embodiments the one or more polymer moiety of -L²- has a Flory radius of at least 3 nm. In certain embodiments the one or more polymer moiety of -L²- has a Flory radius of at least 3.5 nm. In certain embodiments the one or more polymer moiety of -L²- has a Flory radius of at least 4 nm. In certain embodiments the one or more polymer moiety of -L²- has a Flory radius of at least 4.5 nm. In certain embodiments the one or more polymer moiety of -L²- has a Flory radius of at least 5 nm. In certain embodiments the one or more polymer moiety of -L²- has a Flory radius of no more than 200 nm. In certain embodiments the one or more polymer moiety of -L²- has a Flory radius of no more than 175 nm. In certain embodiments the one or more polymer moiety of -L²- has a Flory radius of no more than 150 nm. In certain embodiments the one or more polymer moiety of -L²- has a Flory radius of no more than 125 nm. In certain embodiments the one or more polymer moiety of -L²- has a Flory radius of no more than 100 nm. In certain embodiments the one or more polymer moiety of -L²- has a Flory radius of no more than 75 nm. In certain embodiments the one or more polymer moiety of -L²- has a Flory radius of no more than 50 nm. In certain embodiments the one or more polymer moiety of -L²- has a Flory radius of no more than 45 nm. In certain embodiments the one or more polymer moiety of -L²- has a Flory radius of no more than 40 nm. In certain embodiments the one or more polymer moiety of -L²- has a Flory radius of no more than 35 nm. In certain embodiments the one or more polymer moiety of -L²- has a Flory radius of no more than 30 nm. In certain embodiments the one or more polymer moiety of -L²- has a Flory radius of about 1.2 nm. In certain embodiments the one or more polymer moiety of -L²- has a Flory radius of about 1.5 nm. In certain embodiments the one or more polymer moiety of -L²- has a Flory radius of about 2 nm. In certain embodiments the one or more polymer moiety of -L²- has a Flory radius of about 2.5 nm. In certain embodiments the one or more polymer moiety of -L²- has a Flory radius of about 3 nm. In certain embodiments the one or more polymer moiety of -L²- has a Flory radius of about 3.5 nm. In certain embodiments the one or more polymer moiety of -L²- has a Flory radius of about 4 nm. In certain embodiments the one or more polymer moiety of -L²- has a Flory radius of about 4.5 nm. In certain embodiments the one or more polymer moiety of -L²- has a Flory radius of about 5 nm. In certain embodiments the one or more polymer moiety of -L²- has a Flory radius of about 5.5 nm. In certain embodiments the one or more polymer moiety of -L²- has a Flory radius of about 6 nm. In certain embodiments the one or more polymer moiety of -L²- has a Flory radius of about 6.5 nm. In certain embodiments the one or more polymer moiety of -L²- has a Flory radius of about 7 nm. In certain embodiments the one or more polymer moiety of -L²- has a Flory radius of about 8.5 nm. In certain embodiments the one or more polymer moiety of -L²- has a Flory radius of about 9 nm. In certain embodiments the one or more polymer moiety of -L²- has a Flory radius of about 10 nm. It is understood that if -L²- comprises one polymer moiety the Flory radius provided above applies to this one polymer moiety and if -L²- comprises more than one polymer moiety the Flory radius provided above refers to the Flory radius of all polymer moieties together.

-L²- comprises one or more polymer moiety, such as polymer moiety selected from the group consisting of poly(2-methacryloyl-oxyethyl phosphoyl cholins), poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, alginate, mannans, pectins, rhamnogalacturonans, starches, hydroxyalkyl starches, hydroxyethyl starches and other carbohydrate-based polymers, xylans, and copolymers thereof.

In certain embodiments -L²- comprises a PEG-based polymer. In certain embodiments -L²- comprises a hyaluronic acid-based polymer. In certain embodiments -L²- comprises a random coil polymer. In certain embodiments -L²- comprises a poly-sarcosine polymer.

In certain embodiments the albumin-binding moieties of a compound disclosed herein bind to one albumin, such as via different binding domains. Suitably, the albumin-binding moieties of a compound disclosed herein bind to at least two albumins, such as to two albumins.

In certain embodiments -L¹-L²- is selected from the group consisting

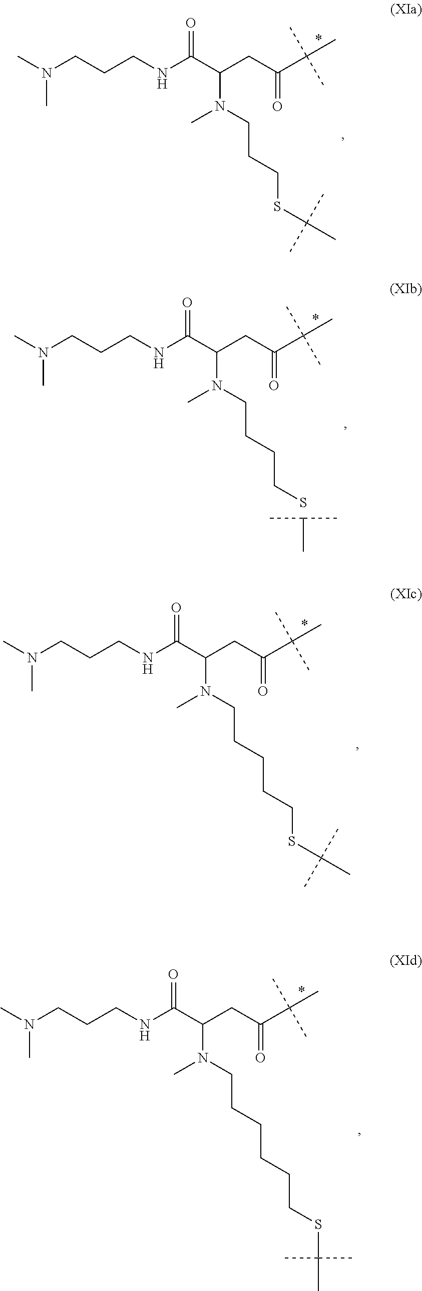

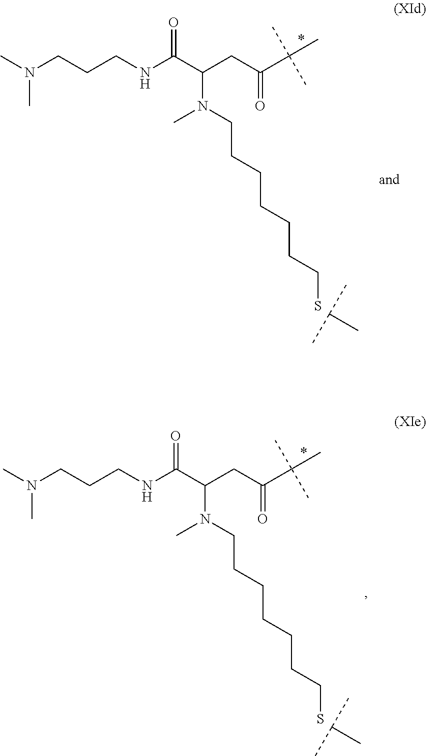

wherein
the dashed line marked with the asterisk indicates attachment to a nitrogen of -D-; and
the unmarked dashed line indicates attachment to the at least one polymeric moiety.

In certain embodiments -L¹-L²- is of formula (XIa). In certain embodiments -L¹-L²- is of formula (XIb). In certain embodiments -L¹-L²- is of formula (XIc). In certain embodiments -L¹-L²- is of formula (XId). In certain embodiments -L¹-L²- is of formula (XIe).

In certain embodiments -L¹-L²- is of formula (XIf).

In certain embodiments -L¹-L²- is selected from the group consisting

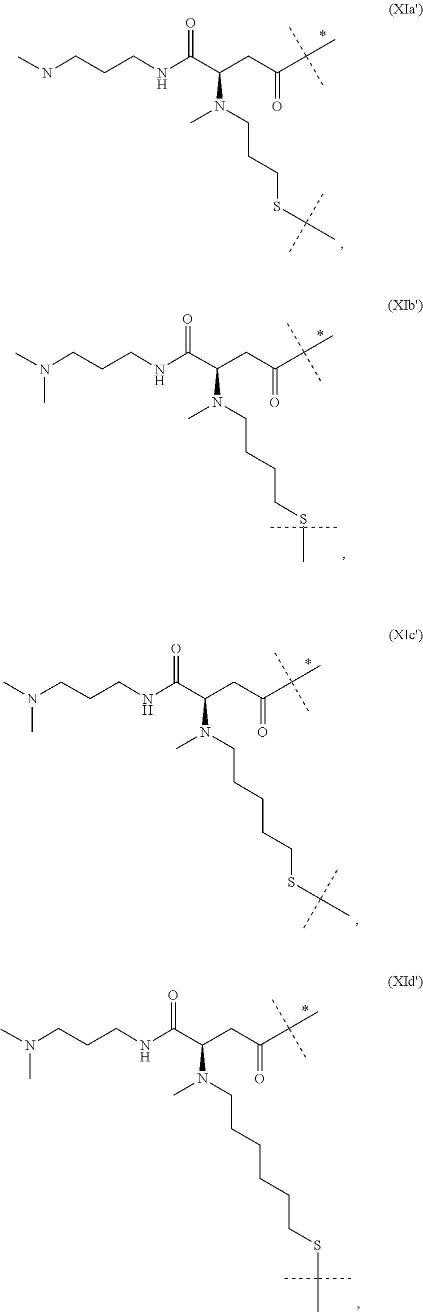

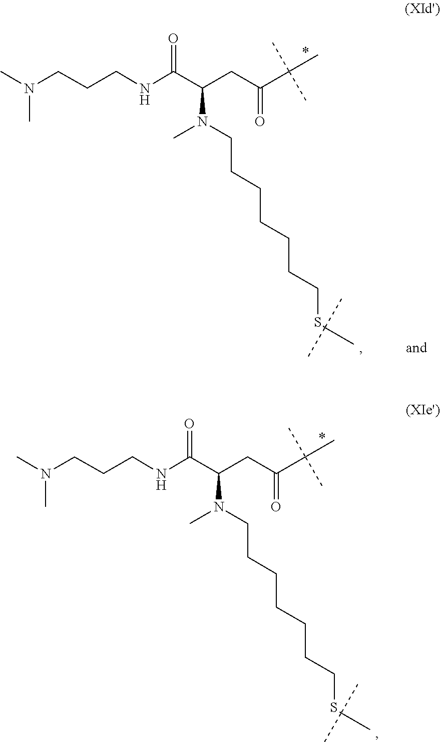

wherein
the dashed line marked with the asterisk indicates attachment to a nitrogen of -D-; and
the unmarked dashed line indicates attachment to the at least one polymeric moiety.

In certain embodiments -L¹-L²- is of formula (Xia′). In certain embodiments -L¹-L²- is of formula (XIb′). In certain embodiments -L¹-L²- is of formula (XIc′). In certain embodiments -L¹-L²- is of formula (XId′). In certain embodiments -L¹-L²- is of formula (XIe′).

In certain embodiments -L¹-L²- is of formula (XIf′).

In certain embodiments -L¹-L²- is selected from the group consisting

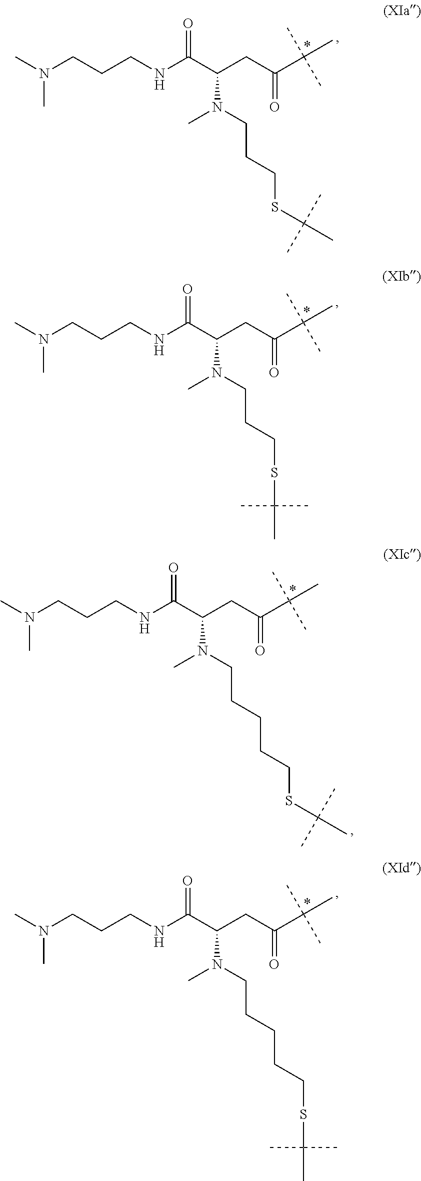

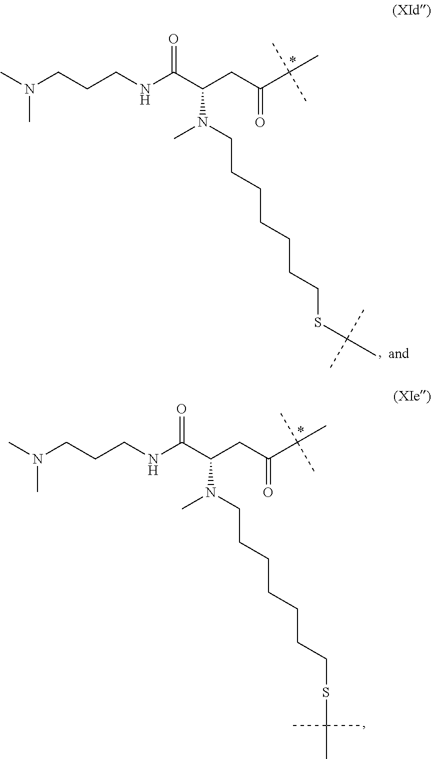

wherein
the dashed line marked with the asterisk indicates attachment to a nitrogen of -D-; and
the unmarked dashed line indicates attachment to the at least one polymeric moiety.

In certain embodiments -L¹-L²- is of formula (Xia″). In certain embodiments -L¹-L²- is of formula (XIb″). In certain embodiments -L¹-L²- is of formula (XIc″). In certain embodiments -L¹-L²- is of formula (XId″). In certain embodiments -L¹-L²- is of formula (XIe″). In certain embodiments -L¹-L²- is of formula (XIf′).

In certain embodiments -L¹-L²- is selected from the group consisting

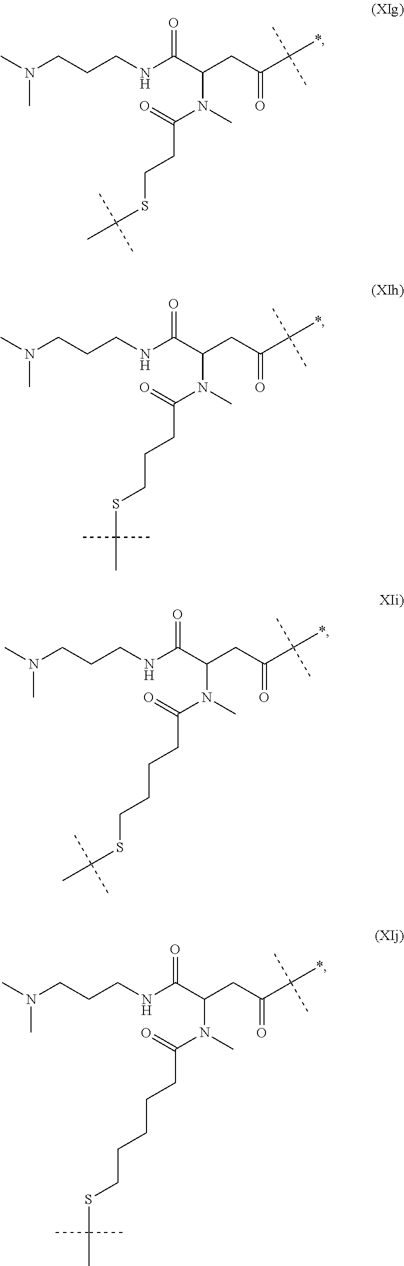

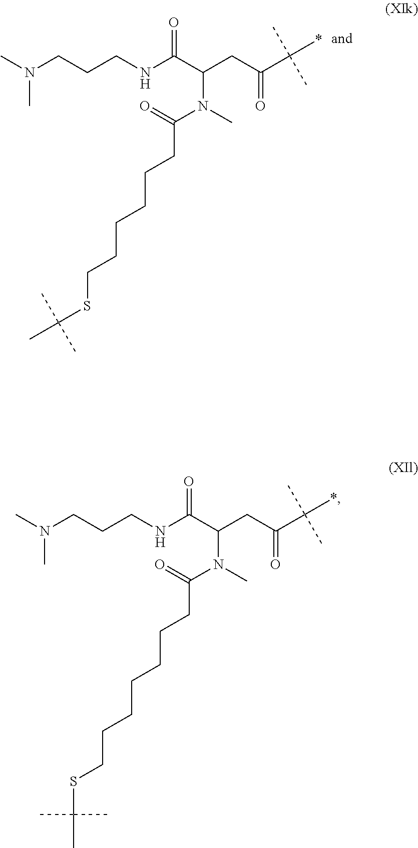

wherein
the dashed line marked with the asterisk indicates attachment to a nitrogen of -D-; and
the unmarked dashed line indicates attachment to the at least one polymeric moiety.

In certain embodiments -L¹-L²- is of formula (XIg). In certain embodiments -L¹-L²- is of formula (XIh). In certain embodiments -L¹-L²- is of formula (XIi). In certain embodiments -L¹-L²- is of formula (XIj). In certain embodiments -L¹-L²- is of formula (XIk).

In certain embodiments -L¹-L²- is of formula (XIl).

In certain embodiments -L¹-L²- is selected from the group consisting

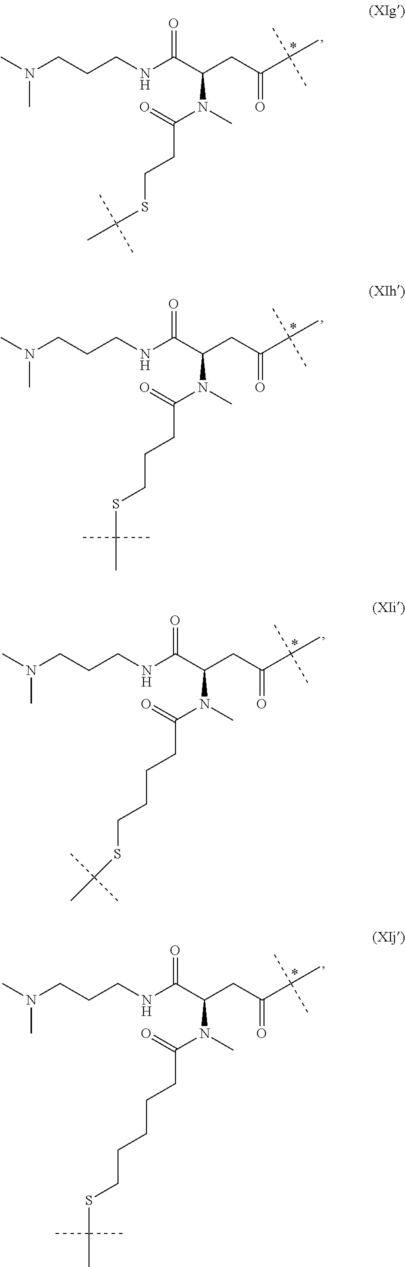

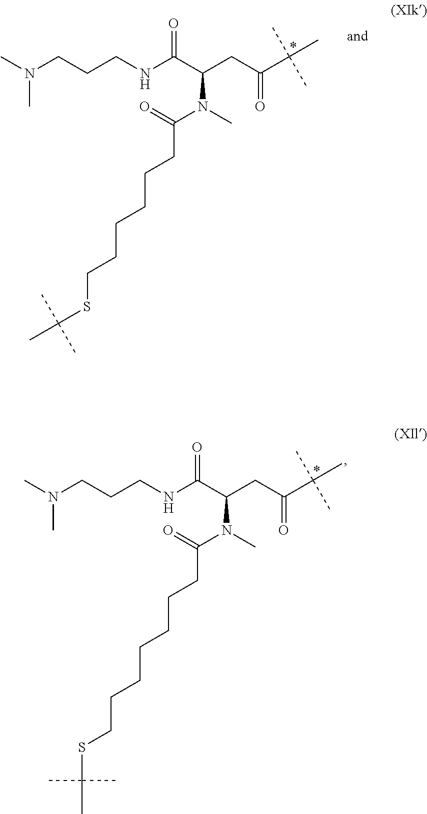

wherein
the dashed line marked with the asterisk indicates attachment to a nitrogen of -D-; and
the unmarked dashed line indicates attachment to the at least one polymeric moiety.

In certain embodiments -L¹-L²- is of formula (XIg′). In certain embodiments -L¹-L²- is of formula (XIh′). In certain embodiments -L¹-L²- is of formula (XIi′). In certain embodiments -L¹-L²- is of formula (XIj′). In certain embodiments -L¹-L²- is of formula (XIk′).

In certain embodiments -L¹-L²- is of formula (XIl′).

In certain embodiments -L¹-L²- is selected from the group consisting

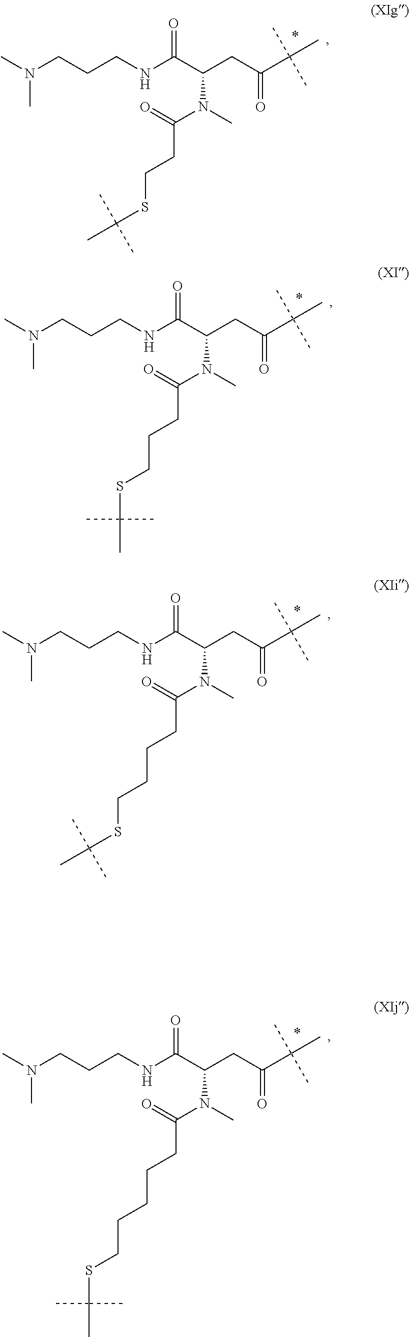

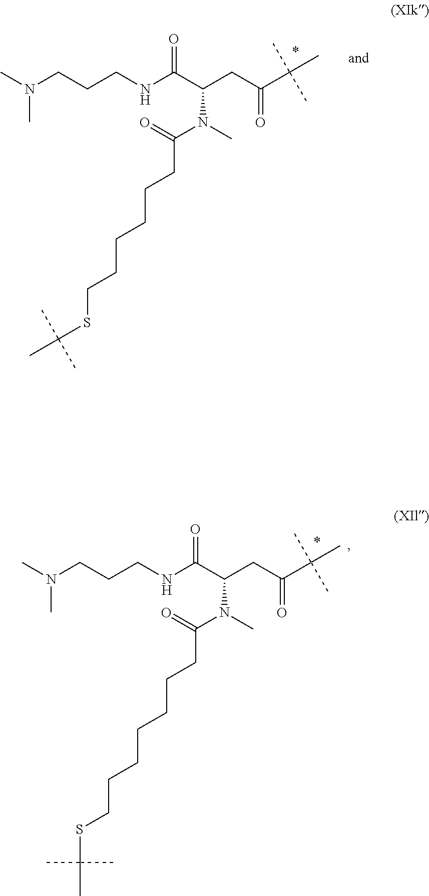

wherein
the dashed line marked with the asterisk indicates attachment to a nitrogen of -D-; and
the unmarked dashed line indicates attachment to the at least one polymeric moiety.

In certain embodiments -L¹-L²- is of formula (XIg″). In certain embodiments -L¹-L²- is of formula (XIh″). In certain embodiments -L¹-L²- is of formula (XIi″). In certain embodiments -L¹-L²- is of formula (XIj″). In certain embodiments -L¹-L²- is of formula (XIk″). In certain embodiments -L¹-L²- is of formula (XIl″).

In certain embodiments -L¹- is a multi-release linker, i.e., a linker from which more than one drug is released. An exemplary class of such multi-release linker is described in the following sections.

In certain embodiments -L^{1′}- is of formula (2a)

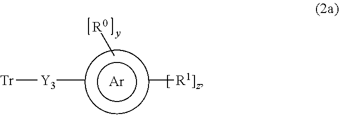

wherein
-Tr is a cleavable triggering moiety;
—Y₃— is independently selected from the group consisting of —O—, —S— and —N(R⁶)—;
z is selected from the group consisting of 2 and 3;
y is selected from the group consisting of 0, 1, 2, 3, 4 and 5;
—Ar— is a ring selected from the group consisting of monocyclic or bicyclic aryl and heteroaryl, provided that —Ar— is connected to —Y₃— and —R¹ via carbon atoms; wherein said monocyclic or bicyclic aryl and heteroaryl are optionally substituted with —R⁰, which are the same or different;
each —R⁰ is independently selected from the group consisting of —C(O)OH, -halogen, —NO₂, —CN, C₁₋₃₀ alkyl, C₂₋₃₀ alkenyl and C₂₋₃₀ alkynyl; wherein C₁₋₃₀ alkyl, C₂₋₃₀ alkenyl and C₂₋₃₀ alkynyl are optionally substituted with one or more —R⁴, which are the same or different; and wherein C₁₋₃₀ alkyl, C₂₋₃₀ alkenyl and C₂₋₃₀ alkynyl are optionally interrupted by one or more groups selected from the group consisting of -T-, —C(O)O—, —O—, —C(O)—, —C(O)N(R⁵)—, —S(O)₂N(R⁵)—, —S(O)N(R⁵)—, —S(O)₂—, —S(O)—, —N(R⁵)S(O)₂N(R^{5a})—, —S—, —N(R⁵)—, —OC(OR⁵)(R^{5a})—, —N(R⁵)C(O)N(R^{5a})— and —OC(O)N(R⁵)—;
each —R¹ is independently selected from the group consisting of

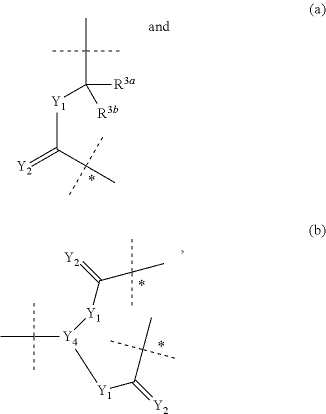

wherein
an unmarked dashed line indicates attachment to —Ar—;
a dashed lined marked with an asterisk indicates attachment to -D-;
the total number of moieties -D- in the compound ranges from 2 to 6;
each —Y₁— is independently selected from the group consisting of —O— and —S—;
each ═Y₂ is independently selected from the group consisting of ═O and ═S;
—Y₄— is

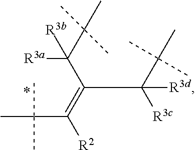

wherein the dashed line marked with the asterisk indicates attachment to —Ar— and the unmarked dashed lines indicate attachment to —Y₁—;
each —R² is independently selected from the group consisting of —H, —C(O)OH, -halogen, —NO₂, —CN, C₁₋₃₀ alkyl, C₂₋₃₀ alkenyl and C₂₋₃₀ alkynyl; wherein C₁₋₃₀ alkyl, C₂₋₃₀ alkenyl and C₂₋₃₀ alkynyl are optionally substituted with one or more —R⁴, which are the same or different; and wherein C₁₋₃₀ alkyl, C₂₋₃₀ alkenyl and C₂₋₃₀ alkynyl are optionally interrupted by one or more groups selected from the group consisting of -T-, —C(O)O—, —O—, —C(O)—, —C(O)N(R⁵)—, —S(O)₂N(R⁵)—, —S(O)N(R⁵)—, —S(O)₂—, —S(O)—, —N(R⁵)S(O)₂N(R^{5a})—, —S—, —N(R⁵)—, —OC(OR⁵)(R^{5a})—, —N(R⁵)C(O)N(R^{5a})— and —OC(O)N(R⁵)—;
each —R^{3a}, R^{3b}, —R^{3c}, —R^{3d} are independently selected from the group consisting of —H, —C(O)OH, —F, —NO₂, —CN, C₁₋₃₀ alkyl, C₂₋₃₀ alkenyl and C₂₋₃₀ alkynyl; wherein C₁₋₃₀ alkyl, C₂₋₃₀ alkenyl and C₂₋₃₀ alkynyl are optionally substituted with one or more —R⁴, which are the same or different; and wherein C₁₋₃₀ alkyl, C₂₋₃₀ alkenyl and C₂₋₃₀ alkynyl are optionally interrupted by one or more groups selected from the group consisting of -T-, —C(O)O—, —O—, —C(O)—, —C(O)N(R⁵)—, —S(O)₂N(R⁵)—, —S(O)N(R⁵)—, —S(O)₂—, —S(O)—, —N(R⁵)S(O)₂N(R^{5a})—, —S—, —N(R⁵)—, —OC(OR⁵)(R^{5a})—, —N(R⁵)C(O)N(R^{5a})— and —OC(O)N(R⁵)—;
each -T- is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each -T- is independently optionally substituted with one or more —R⁴, which are the same or different;
—R⁴ is selected from the group consisting of —NO₂, —OCH₃, —CN, —N(R⁵)(R^{5a}), —OH, —C(O)OH and C₁₋₆ alkyl; wherein C₁₋₆ alkyl is optionally substituted with one or more halogen, which are the same or different;
—R⁵ and —R^{5a} are independently selected from the group consisting of —H and C₁₋₆ alkyl; wherein C₁₋₆ alkyl is optionally substituted with one or more halogen, which are the same or different;
each —R⁶ is independently selected from the group consisting of —H and C₁₋₆ alkyl;
and wherein -L^{1′}- is substituted with one moiety -L²-.

In certain embodiments all —R¹ of formula (2a) are of formula (a).

In certain embodiments all —R¹ of formula (2a) are of formula (b).

In certain embodiments z of formula (2a) is 2. In certain embodiments z of formula (2a) is 3.

In certain embodiments z of formula (2a) is 2 and both —R¹ are of formula (a). In certain embodiments z of formula (2a) is 2 and both —R¹ are of formula (b). In certain embodiments z is of formula (2a) is 2 and one —R¹ is of formula (a) and one —R¹ is of formula (b).

In certain embodiments z of formula (2a) is 3 and all —R¹ are of formula (a). In certain embodiments z of formula (2a) is 3 and all —R¹ are of formula (b). In certain embodiments z of formula (2a) is 3 and one —R¹ is of formula (a) and two —R¹ are of formula (b). In certain embodiments z of formula (2a) is 3 and two —R¹ are of formula (a) and one —R¹ is of formula (b).

In certain embodiments y of formula (2a) is 0. In certain embodiments y of formula (2a) is 1. In certain embodiments y of formula (2a) is 2. In certain embodiments y of formula (2a) is 3. In certain embodiments y of formula (2a) is 4. In certain embodiments y of formula (2a) is 5.

In certain embodiments z of formula (2a) is 2, both —R¹ of formula (2a) are of formula (a) and y of formula (2a) is 0. In certain embodiments z of formula (2a) is 3, all —R¹ of formula (2a) are of formula (a) and y of formula (2a) is 0. In certain embodiments z of formula (2a) is 3, two —R¹ are of formula (a), one —R¹ of formula (2a) is of formula (b) and y of formula (2a) is 0.

In certain embodiments —Y₁— of formula (2a) is —O—. In certain embodiments —Y₁— of formula (2a) is —S—.

In certain embodiments ═Y₂ of formula (2a) is ═O. In certain embodiments ═Y₂ of formula (2a) is =S.

In certain embodiments —Y₃— of formula (2a) is —O—. In certain embodiments —Y₃— of formula (2a) is —S—. In certain embodiments —Y₃— of formula (2a) is —N(R⁶)—, wherein —R⁶ is as described elsewhere within the section covering the exemplary multi-release linker.

In certain embodiments -Tr of formula (2a) is selected from the group consisting of a peptidyl moiety;

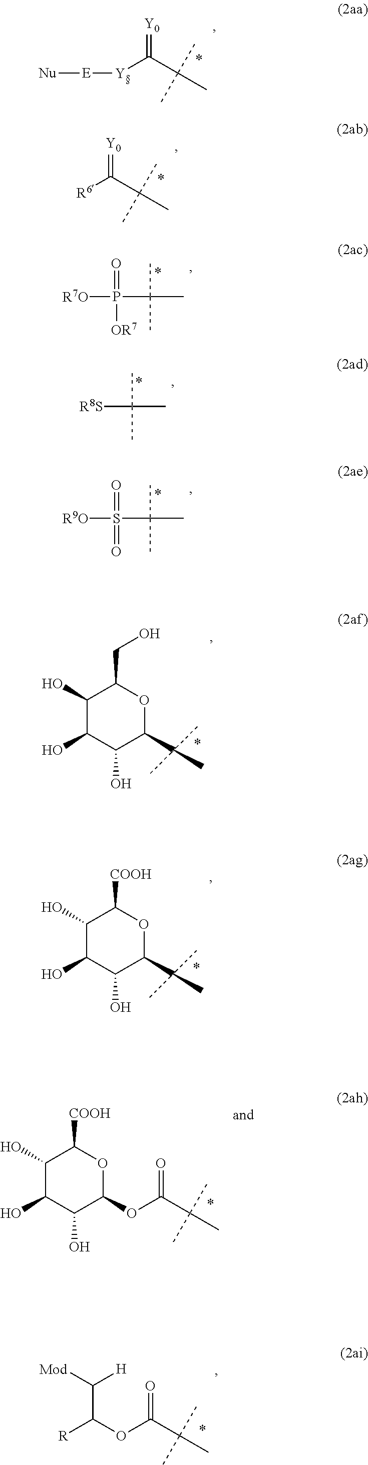

wherein
the dashed line marked with an asterisk indicates attachment to —Y³—;
-Nu is a nucleophile;
—Y_{§} — is selected from the group consisting of —O—, —C(R¹⁰)(R^{10a})—, —N(R⁶)— and —S—;
═Y₀ is selected from the group consisting of ═O, ═S and ═N(R^{5′});
-E- and —R are independently selected from the group consisting of C₁₋₆ alkyl, C₂₋₆ alkenyl, C₂₋₆ alkynyl and -Q-; wherein C₁₋₆ alkyl, C₂₋₆ alkenyl, C₂₋₆ alkynyl are optionally substituted with one or more —R¹¹, which are the same or different; and wherein C₁₋₆ alkyl, C₂₋₆ alkenyl, C₂₋₆ alkynyl are optionally interrupted by one or more groups selected from the group consisting of -Q-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)R^{12a}—, —N(R¹²)C(O)N(R^{12a})— and —OC(O)N(R¹²)—;
-Mod is selected from the group consisting of CF₃PhSO₂—, ClPhSO₂—, PhSO₂—, MePhSO₂—, MeOPhSO₂—, 2,4,6-Me₃PhSO₂—, MeSO₂—, O(CH₂CH₂)₂N—SO₂—, CN— and Et₂NSO₂—;
—R⁶ is selected from the group consisting of —H and C₁₋₆ alkyl;
—R⁵″, —R^{6′}, —R⁷, —R⁸, —R⁹, —R¹⁰, —R^{10a} are independently selected from the group consisting of —H, C₁₋₂₀ alkyl, C₂₋₂₀ alkenyl, C₂₋₂₀ alkynyl and -Q; wherein C₁₋₂₀ alkyl, C₂₋₂₀ alkenyl and C₂₋₂₀ alkynyl are optionally substituted with one or more —R¹¹, which are the same or different; and wherein C₁₋₂₀ alkyl, C₂₋₂₀ alkenyl and C₂₋₂₀ alkynyl are optionally interrupted by one or more groups selected from the group consisting of -Q-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)R^{12a}—, —N(R¹²)C(O)N(R^{12a})— and —OC(O)N(R¹²)—,
each Q is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each Q is independently optionally substituted with one or more —R¹¹, which are the same or different; and
—R¹¹ is selected from the group consisting of C₁₋₆ alkyl and —N₃, wherein C₁₋₆ alkyl is optionally substituted with one or more halogen, which are the same or different; and
—R¹² and —R^{12a} are independently selected from the group consisting of —H and C₁₋₆ alkyl; wherein C₁₋₆ alkyl is optionally substituted with one or more halogen, which are the same or different.

-Nu of formula (2aa) is a nucleophile that may be selected from the group consisting of primary, secondary, or tertiary amine and amide. In certain embodiments -Nu of formula (2aa) is selected from the group consisting of secondary and tertiary amine. In certain embodiments -Nu of formula (2aa) is a primary amine. In certain embodiments -Nu of formula (2aa) is a secondary amine. In certain embodiments -Nu of formula (2aa) is a tertiary amine. In certain embodiments -Nu of formula (2aa) is an amide.

In certain embodiments —Y_{§} — of formula (2aa) is selected from the group consisting of —O—, —C(R¹⁰)(R^{10a})— and —N(R⁶)—, wherein —R¹⁰, —R^{10a} and —R⁶. In certain embodiments —Y_{§} — of formula (2aa) is —O—.

In certain embodiments —Y_{§} — of formula (2aa) is —C(R¹⁰)(R^{10a})—, wherein —R¹⁰, —R^{10a} are independently selected from the group consisting of —H, C₁₋₂₀ alkyl, C₂₋₂₀ alkenyl, C₂₋₂₀ alkynyl and -Q; wherein C₁₋₂₀ alkyl, C₂₋₂₀ alkenyl and C₂₋₂₀ alkynyl are optionally substituted with one or more —R¹¹, which are the same or different; and wherein C₁₋₂₀ alkyl, C₂₋₂₀ alkenyl and C₂₋₂₀ alkynyl are optionally interrupted by one or more groups selected from the group consisting of -Q-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)R^{12a}—, —N(R¹²)C(O)N(R^{12a})—, and —OC(O)N(R¹²)—;
each Q is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each Q is independently optionally substituted with one or more —R¹¹, which are the same or different; and
—R¹¹ is selected from the group consisting of C₁₋₆ alkyl and —N₃, wherein C₁₋₆ alkyl is optionally substituted with one or more halogen, which are the same or different; and
—R¹² and —R^{12a} are independently selected from the group consisting of —H and C₁₋₆ alkyl; wherein C₁₋₆ alkyl is optionally substituted with one or more halogen, which are the same or different

In certain embodiments —Y_{§} — of formula (2aa) is —N(R⁶)—, wherein —R⁶ is selected from the group consisting of —H and C₁₋₆ alkyl.

In certain embodiments ═Y₀ of formula (2aa) is selected from the group consisting of ═O and ═S. In certain embodiments ═Y₀ of formula (2aa) is ═O. In certain embodiments ═Y₀ of formula (2aa) is ═S.

In certain embodiments ═Y₀ of formula (2aa) is ═N(R′), wherein —R⁵ is selected from the group consisting of —H, C₁₋₂₀ alkyl, C₂₋₂₀ alkenyl, C₂₋₂₀ alkynyl and -Q; wherein C₁₋₂₀ alkyl, C₂₋₂₀ alkenyl and C₂₋₂₀ alkynyl are optionally substituted with one or more —R¹¹, which are the same or different; and wherein C₁₋₂₀ alkyl, C₂₋₂₀ alkenyl and C₂₋₂₀ alkynyl are optionally interrupted by one or more groups selected from the group consisting of -Q-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)R^{12a}—, N(R¹²)C(O)N(R^{12a})— and —OC(O)N(R¹²)—;
each Q is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each Q is independently optionally substituted with one or more —R¹¹, which are the same or different; and
—R¹¹ is selected from the group consisting of C₁₋₆ alkyl and —N₃, wherein C₁₋₆ alkyl is optionally substituted with one or more halogen, which are the same or different; and
—R¹² and —R^{12a} are independently selected from the group consisting of —H and C₁₋₆ alkyl; wherein C₁₋₆ alkyl is optionally substituted with one or more halogen, which are the same or different

In certain embodiments -E- of formula (2aa) is selected from the group consisting of C₁₋₆ alkyl, C₂₆ alkenyl and C₂₆ alkynyl. In certain embodiments -E- of formula (2aa) is C₁₋₆ alkyl. In certain embodiments -E- of formula (2aa) is C₂₋₆ alkenyl. In certain embodiments -E- of formula (2aa) is C₂₋₆ alkynyl. In certain embodiments -E- of formula (2aa) is -Q-, which in certain embodiments is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments Q is phenyl. In certain embodiments Q is naphthyl. In certain embodiments Q is indenyl. In certain embodiments Q is indanyl. In certain embodiments Q is tetralinyl. In certain embodiments Q is C₃₋₁₀ cycloalkyl. In certain embodiments Q is 3- to 10-membered heterocyclyl. In certain embodiments Q is 8- to 11-membered heterobicyclyl. In certain embodiments Q is substituted with one or more —R¹¹. In certain embodiments Q is not substituted with —R¹¹.

In certain embodiments —R^{5′} of formula (2aa) is selected from the group consisting of —H, C₁₋₆ alkyl, C₂₋₆ alkenyl and C₂₋₆ alkynyl. In certain embodiments —R^{5′} of formula (2aa) is selected from the group consisting of —H and C₁₋₆ alkyl. In certain embodiments —R^{5′} of formula (2aa) is —H. In certain embodiments —R^{5′} of formula (2aa) is C₁₋₆ alkyl.

In certain embodiments —R^{6′}, —R⁷, —R⁸, —R⁹, —R¹⁰, —R^{10a} as used in the various embodiments for Tr are independently selected from the group consisting of —H, C₁₋₁₀ alkyl, C₂₋₁₀ alkenyl, C₂₋₁₀ alkynyl and -Q, wherein -Q is as defined elsewhere within the section covering the exemplary multi-release linker.

In certain embodiments —R⁷ and —R⁹ as used in the various embodiments for Tr are independently selected from the group consisting of —H and C₁₋₆ alkyl. In certain embodiments both —R⁷ and —R⁹ are —H. In certain embodiments —R⁷ is —H. In certain embodiments —R⁷ is C₁₋₆ alkyl. In certain embodiments —R⁹ is —H. In certain embodiments —R⁹ is C₁₋₆ alkyl.

In certain embodiments —R¹⁰ and —R^{10a} of formula (2aa) are independently selected from the group consisting of —H and C₁₋₆ alkyl. In certain embodiments both —R¹⁰ and —R^{10a} of formula (2aa) are —H. In certain embodiments —R¹⁰ of formula (2aa) is —H. In certain embodiments —R¹⁰ of formula (2aa) is C₁₋₆ alkyl. In certain embodiments —R^{10a} of formula (2aa) is —H. In certain embodiments —R^{10a} of formula (2aa) is C₁₋₆ alkyl.

In certain embodiments -Tr of formula (2a) is of formula (2aa)

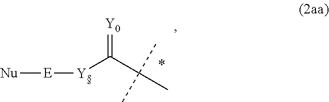

wherein
the dashed line marked with an asterisk indicates attachment to —Y₃—; and
-Nu, -E-, —Y_{§} — and ═Y₀ are as defined elsewhere within the section covering the exemplary multi-release linker herein.

It is understood that in this instance the release of the drug is not triggered by an enzyme, and that the drug is released in its unmodified, pharmacologically active form in the absence of an enzyme.

In certain embodiments -Tr of formula (2a) is

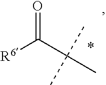

wherein and the dashed line marked with an asterisk indicates attachment to —Y³—; and
—R^{6′} is as defined elsewhere within the section covering the exemplary multi-release linker herein.

In certain embodiments —R^{6′} is

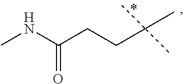

wherein the dashed line marked with the asterisk indicates attachment to the carbon atom substituted by the oxygen atom.

In certain embodiments —R^{6′} is of formula (a′):

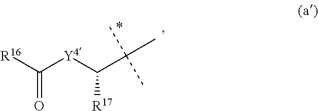

wherein —Y^{4′}— is selected from the group consisting of C₃₋₁₀ cycloalkyl, 3- to 10-membered heterocyclyl and 8- to 11-membered heterobicyclyl, which are optionally substituted with one or more —R¹⁸ which are the same or different;
—R¹⁶ and —R¹⁷ are independently selected from the group consisting of —H, C₁₋₁₀ alkyl, C₂₋₁₀ alkenyl and C₂₋₁₀ alkynyl; wherein C₁₋₁₀ alkyl, C₂₋₁₀ alkenyl and C₂₋₁₀ alkynyl are optionally substituted with one or more —R¹⁸ which are the same or different; and wherein C₁₋₁₀ alkyl, C₂₋₁₀ alkenyl and C₂₋₁₀ alkynyl are optionally interrupted by one or more groups selected from the group consisting of -A′-, —C(O)O—, —O—, —C(O)—, —C(O)N(R¹⁹)—, —S(O)₂N(R¹⁹), —S(O)N(R¹⁹)—, —S(O)₂—, —S(O)—, —N(R¹⁹)S(O)₂N(R^{19a})—, —S—, —N(R¹⁹)—, —OC(OR¹⁹)R^{19a}—, —N(R¹⁹)C(O)N(R^{19a})—, —OC(O)N(R¹⁹)— and —N(R¹⁹)C(NH₂)N(R^{19a})—;
each A′ is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each A′ is independently optionally substituted with one or more —R¹⁸ which are the same or different;
wherein —R¹⁸, —R¹⁹ and —R^{19a} are independently selected from the group consisting of —H and C₁₋₆ alkyl; wherein C₁₋₆ alkyl is optionally substituted with one or more halogen, which are the same or different; and
wherein the dashed line marked with an asterisk indicates attachment to the rest of -Tr.

In certain embodiments —Y^{4′}- is selected from the group consisting of C₃₋₁₀ cycloalkyl, 3- to 10-membered heterocyclyl and 8- to 11-membered heterobicyclyl. In certain embodiments —Y^{4′}— is C₃₋₁₀ cycloalkyl. In certain embodiments —Y^{4′}— is 3- to 10-membered heterocyclyl. In certain embodiments —Y^{4′}— is 8- to 11-membered heterobicyclyl. In certain embodiments —Y^{4′}— is substituted with one or more —R¹⁸ which are the same or different. In certain embodiments —Y^{4′}— is not substituted with —R¹⁸.

In certain embodiments —R¹⁶ and —R¹⁷ are selected from the group consisting of C₁₋₁₀ alkyl, C₂₋₁₀ alkenyl and C₂₋₁₀ alkynyl. In certain embodiments —R¹⁶ is C₁₋₁₀ alkyl. In certain embodiments —R¹⁶ is C₂₋₁₀ alkenyl. In certain embodiments —R¹⁶ is C₂₋₁₀ alkynyl. In certain embodiments —R¹⁷ is C₁₋₁₀ alkyl. In certain embodiments —R¹⁷ is C₂₋₁₀ alkenyl. In certain embodiments —R¹⁷ is C₂₋₁₀ alkynyl.

In certain embodiments A′ is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments A′ is phenyl. In certain embodiments A′ is naphthyl.

In certain embodiments A′ is indenyl. In certain embodiments A′ is indanyl. In certain embodiments A′ is tetralinyl. In certain embodiments A′ is C₃₋₁₀ cycloalkyl. In certain embodiments A′ is 3- to 10-membered heterocyclyl. In certain embodiments A′ is 8- to 11-membered heterobicyclyl. In certain embodiments A′ is substituted with one or more —R¹⁸, which are the same or different. In certain embodiments A′ is not substituted with —R¹⁸.

In certain embodiments —R¹⁸, —R¹⁹ and —R^{19a} are selected from the group consisting of —H and C₁₋₆ alkyl. In certain embodiments —R¹⁸ is —H. In certain embodiments —R¹⁸ is C₁₋₆ alkyl. In certain embodiments —R¹⁹ is —H. In certain embodiments —R¹⁹ is C₁₋₆ alkyl. In certain embodiments —R^{19a} is —H. In certain embodiments —R^{19a} is C₁₋₆ alkyl.

In certain embodiments —R^{6′} is of formula (b′):

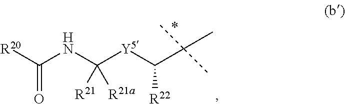

wherein —Y^{5′}— is selected from the group consisting of -Q′-, C₁₋₁₀ alkyl, C₂₋₁₀ alkenyl and C₂₋₁₀ alkynyl; wherein C₁₋₁₀ alkyl, C₂₋₁₀ alkenyl and C₂₋₁₀ alkynyl are optionally substituted with one or more —R²³, which are the same or different; and wherein C₁₋₁₀ alkyl, C₂₋₁₀ alkenyl and C₂₋₁₀ alkynyl are optionally interrupted by one or more groups selected from the group consisting of -Q′-, —C(O)O—, —O—, —C(O)—, —C(O)N(R²⁴)—, —S(O)₂N(R²⁴)—, —S(O)N(R²⁴)—, —S(O)₂—, —S(O)—, —N(R²⁴)S(O)₂N(R^{24a})—, —S—, —N(R²⁴)—, —OC(OR²⁴)R^{24a}—,
—N(R²⁴)C(O)N(R^{24a})—, —OC(O)N(R²⁴)— and —N(R²⁴)C(NH₂)N(R^{24a})—;
—R²⁰, —R²¹, —R^{21a} and —R²² are independently selected from the group consisting of —H, C₁₋₁₀ alkyl, C₂₋₁₀ alkenyl and C₂₋₁₀ alkynyl; wherein C₁₋₁₀ alkyl, C₂₋₁₀ alkenyl and C₂₋₁₀ alkynyl are optionally substituted with one or more —R²³ which are the same or different; and wherein C₁₋₁₀ alkyl, C₂₋₁₀ alkenyl and C₂₋₁₀ alkynyl are optionally interrupted by one or more groups selected from the group consisting of -Q′-, —C(O)O—, —O—, —C(O)—, —C(O)N(R²⁴)—, —S(O)₂N(R²⁴)—, —S(O)N(R²⁴)—, —S(O)₂—, —S(O)—, —N(R²⁴)S(O)₂N(R^{24a})—, —S—, —N(R²⁴)—, —OC(OR²⁴)R^{24a}—, —N(R²⁴)C(O)N(R^{24a})—, —OC(O)N(R²⁴)— and —N(R²⁴)C(NH₂)N(R^{24a})—;
each Q′ is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each Q′ is independently optionally substituted with one or more —R²³, which are the same or different;
wherein —R²³, —R²⁴ and —R^{24a} are independently selected from the group consisting of —H and C₁₋₆ alkyl; wherein C₁₋₆ alkyl is optionally substituted with one or more halogen, which are the same or different;
optionally, the pair —R²¹/—R^{21a} is joined together with the atoms to which is attached to form a C₃₋₁₀ cycloalkyl, 3- to 10-membered heterocyclyl or an 8- to 11-membered heterobicyclyl; and
wherein the dashed line marked with an asterisk indicates attachment to the rest of -Tr.

In certain embodiments —Y^{5′}— is selected from the group consisting of -Q′-, C₁₋₁₀ alkyl, C₂₋₁₀ alkenyl and C₂₋₁₀ alkynyl. In certain embodiments —Y^{5′}— is -Q′-. In certain embodiments —Y^{5′}— is C₁₋₁₀ alkyl. In certain embodiments —Y^{5′}— is C₂₋₁₀ alkenyl. In certain embodiments —Y^{5′}— is C₂₋₁₀ alkynyl.

In certain embodiments Q′ is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments Q′ is phenyl. In certain embodiments Q′ is naphthyl. In certain embodiments Q′ is indenyl. In certain embodiments Q′ is indanyl. In certain embodiments Q′ is C₃₋₁₀ cycloalkyl. In certain embodiments Q′ is 3- to 10-membered heterocyclyl. In certain embodiments Q′ is 8- to 11-membered heterobicyclyl. In certain embodiments Q′ is substituted with one or more —R²³ which are the same or different. In certain embodiments Q′ is not substituted with —R²³.

In certain embodiments —R²⁰, —R²¹, —R^{21a} and —R²² are selected from the group consisting of —H, C₁₋₁₀ alkyl, C₂₋₁₀ alkenyl and C₂₋₁₀ alkynyl. In certain embodiments —R²⁰ is —H. In certain embodiments —R²⁰ is C₁₋₁₀ alkyl. In certain embodiments —R²⁰ is C₂₋₁₀ alkenyl. In certain embodiments —R²⁰ is C₂₋₁₀ alkynyl. In certain embodiments —R²¹ is —H. In certain embodiments —R²¹ is C₁₋₁₀ alkyl. In certain embodiments —R²¹ is C₂₋₁₀ alkenyl. In certain embodiments —R²¹ is C₂₋₁₀ alkynyl. In certain embodiments —R^{21a} is —H. In certain embodiments —R^{21a} is C₁₋₁₀ alkyl. In certain embodiments —R^{21a} is C₂₋₁₀ alkenyl. In certain embodiments —R^{21a} is C₂₋₁₀ alkynyl. In certain embodiments —R²² is —H. In certain embodiments —R²² is C₁₋₁₀ alkyl. In certain embodiments —R²² is C₂₋₁₀ alkenyl. In certain embodiments —R²² is C₂₋₁₀ alkynyl.

In certain embodiments —R²³, —R²⁴ and —R^{24a} are selected from the group consisting of —H and C₁₋₆ alkyl. In certain embodiments —R²³ is —H. In certain embodiments —R²³ is C₁₋₆ alkyl. In certain embodiments —R²⁴ is —H. In certain embodiments —R²⁴ is C₁₋₆ alkyl. In certain embodiments —R^{24a} is —H. In certain embodiments —R^{24a} is C₁₋₆ alkyl.

In certain embodiments the pair —R²¹/—R^{21a} is joined together with the atoms to which is attached to form a C₃₋₁₀ cycloalkyl.

In certain embodiments —R^{6′} is of formula (c)

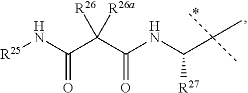

wherein
—R²⁵, —R²⁶, —R^{26a} and —R²⁷ are independently selected from the group consisting of —H, C₁₋₁₀ alkyl, C₂₋₁₀ alkenyl and C₂₋₁₀ alkynyl; wherein C₁₋₁₀ alkyl, C₂₋₁₀ alkenyl and C₂₋₁₀ alkynyl are optionally substituted with one or more —R²⁸ which are the same or different; and wherein C₁₋₁₀ alkyl, C₂₋₁₀ alkenyl and C₂₋₁₀ alkynyl are optionally interrupted by one or more groups selected from the group consisting of -Q*-, —C(O)O—, —O—, —C(O)—, —C(O)N(R²⁹)—, —S(O)₂N(R²⁹)—, —S(O)N(R²⁹)—, —S(O)₂—, —S(O)—, —N(R²⁹)S(O)₂N(R^{29a})—, —S—, —N(R²⁹)—, —OC(OR²⁹)R^{29a}—, —N(R²⁹)C(O)N(R^{29a})—, —OC(O)N(R²⁹)— and —N(R²⁹)C(NH₂)N(R^{29a})—;
each Q* is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each Q* is independently optionally substituted with one or more —R²⁸, which are the same or different;
wherein —R²⁸, —R²⁹ and —R^{29a} are independently selected from the group consisting of —H and C₁₋₆ alkyl; wherein C₁₋₆ alkyl is optionally substituted with one or more halogen, which are the same or different;
optionally, the pair —R²⁶/—R^{26a} is joined together with the atoms to which is attached to form a C₃₋₁₀ cycloalkyl, 3- to 10-membered heterocyclyl or an 8- to 11-membered heterobicyclyl; and
wherein the dashed line marked with an asterisk indicates attachment to the rest of -Tr.

In certain embodiments —R²⁵, —R²⁶, —R^{26a} and —R²⁷ are selected from the group consisting of —H, C₁₋₁₀ alkyl, C₂₋₁₀ alkenyl and C₂₋₁₀ alkynyl. In certain embodiments —R²⁵ is —H. In certain embodiments —R²⁵ is C₁₋₁₀ alkyl. In certain embodiments —R²⁵ is C₂₋₁₀ alkenyl. In certain embodiments —R²⁵ is C₂₋₁₀ alkynyl. In certain embodiments —R²⁶ is —H. In certain embodiments —R²⁶ is C₁₋₁₀ alkyl. In certain embodiments —R²⁶ is C₂₋₁₀ alkenyl. In certain embodiments —R²⁶ is C₂₋₁₀ alkynyl. In certain embodiments —R^{26a} is —H. In certain embodiments —R^{26a} is C₁₋₁₀ alkyl.

In certain embodiments —R^{26a} is C₂₋₁₀ alkenyl. In certain embodiments —R^{26a} is C₂₋₁₀ alkynyl. In certain embodiments —R²⁷ is —H. In certain embodiments —R²⁷ is C₁₋₁₀ alkyl. In certain embodiments —R²⁷ is C₂₋₁₀ alkenyl. In certain embodiments —R²⁷ is C₂₋₁₀ alkynyl.

In certain embodiments Q* is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments Q* is phenyl. In certain embodiments Q* is naphthyl. In certain embodiments Q* is indenyl. In certain embodiments Q* is indanyl. In certain embodiments Q* is tetralinyl. In certain embodiments Q* is C₃₋₁₀ cycloalkyl. In certain embodiments Q* is 3- to 10-membered heterocyclyl. In certain embodiments Q* is 8-to 11-membered heterobicyclyl. In certain embodiments Q* is substituted with one or more —R²⁸, which are the same or different. In certain embodiments Q* is not substituted with —R²⁸.

In certain embodiments —R²⁸, —R²⁹ and —R^{29a} are selected from the group consisting of —H and C₁₋₆ alkyl. In certain embodiments —R²⁸ is —H. In certain embodiments —R²⁸ is C₁₋₆ alkyl. In certain embodiments —R²⁹ is —H. In certain embodiments —R²⁹ is C₁₋₆ alkyl. In certain embodiments —R^{29a} is —H. In certain embodiments —R^{29a} is C₁₋₆ alkyl.

In certain embodiments the pair —R²⁶/—R^{26a} is joined together with the atoms to which is attached to form a C₃₋₁₀ cycloalkyl. In certain embodiments the pair —R²⁶/—R^{26a} is joined together with the atoms to which is attached to form a cyclobutyl.

In certain embodiments -Tr is

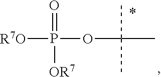

wherein the dashed line marked with an asterisk indicates attachment to —Y³— and —R⁷ is defined as elsewhere within the section covering the exemplary multi-release linker herein. In this instance the release of the drug may be triggered by an enzyme, such as phosphatase.

In certain embodiments -Tr is

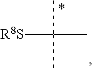

wherein the dashed line marked with an asterisk indicates attachment to —Y³— which is —S— and —R⁸ is as defined elsewhere within the section covering the exemplary multi-release linker herein.

In certain embodiments -Tr is

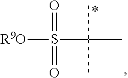

wherein the dashed line marked with an asterisk indicates attachment to —Y³— and —R⁹ is as defined elsewhere within the section covering the exemplary multi-release linker herein. In this instance the release of the drug may be triggered by an enzyme, such as sulfatase.

In certain embodiments -Tr is

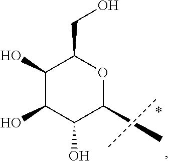

wherein the dashed line marked with an asterisk indicates attachment to —Y³—. In this instance the release of the drug may be triggered by an enzyme, such as a-galactosidase.

In certain embodiments -Tr is

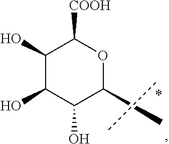

wherein the dashed line marked with an asterisk indicates attachment to —Y³—. In this instance the release of the drug may be triggered by an enzyme, such as (3-glucuronidase.

In certain embodiments -Tr is

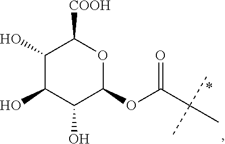

wherein the dashed line marked with an asterisk indicates attachment to —Y³—. In this instance the release of the drug may be triggered by an enzyme, such as (3-glucuronidase.

In certain embodiments -Tr is a peptidyl moiety. It is understood that if -Tr is a peptidyl moiety, then the release of the -D may be triggered by an enzyme, such as protease. In certain embodiments the protease is selected from the group consisting of cathepsin B and cathepsin K. In certain embodiments the protease is cathepsin B. In certain embodiments the protease is cathepsin K.

In certain embodiments -Tr is a peptidyl moiety, such as a dipeptidyl, tripeptidyl, tetrapeptidyl, pentapeptidyl or hexapeptidyl moiety. In certain embodiments -Tr is a dipeptidyl moiety. In certain embodiments -Tr is a tripeptidyl moiety. In certain embodiments -Tr is a tetrapeptidyl moiety. In certain embodiments -Tr is a pentapeptidyl moiety. In certain embodiments -Tr is a hexapeptidyl moiety.

In certain embodiments -Tr is a peptidyl moiety selected from the group consisting of:

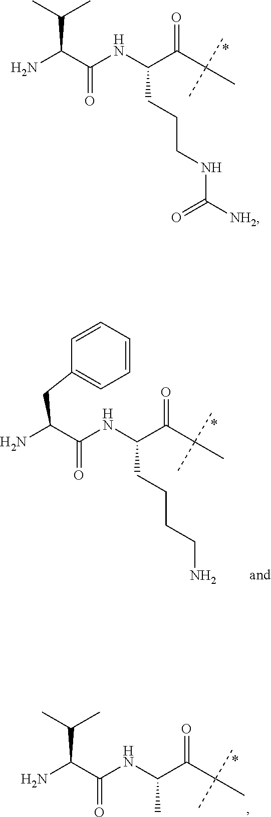

wherein the dashed line marked with an asterisk indicates attachment to —Y³—.

In certain embodiments -Tr is

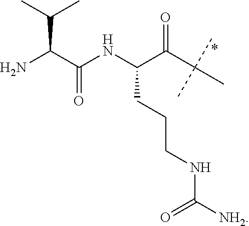

In certain embodiments -Tr is

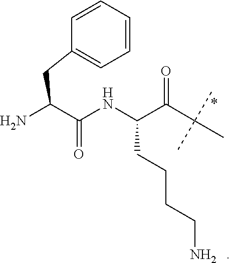

In certain embodiments -Tr is

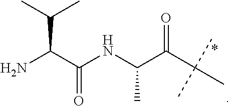

In certain embodiments —Y₃— is —N(R⁶)— and -Tr has the structure of -L¹- as disclosed in WO 2011/012722 A1, which is herewith incorporated by reference. Accordingly, In certain embodiments -Tr is of formula (a012):

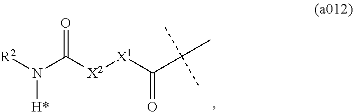

wherein the dashed line indicates attachment —Y₃—;
—X¹— is —C(R¹R^{1a})— or a cyclic fragment selected from C₃₋₇ cycloalkyl, 4 to 7 membered heterocyclyl, phenyl, naphthyl, indenyl, indanyl, tetralinyl, or 9 to 11 membered heterobicyclyl,
wherein in case —X¹— is a cyclic fragment, said cyclic fragment is incorporated into the moiety of formula (a012) via two adjacent ring atoms and the ring atom of —X¹—, which is adjacent to the carbon atom of the amide bond, is also a carbon atom;
—X²— is a chemical bond or selected from —C(R³R^{3a})—, —N(R³)—, —O—, —C(R³R^{3a})—, C(R⁴R^{4a})—, —C(R³R^{3a})—N(R⁴)—, —N(R³)—C(R⁴R^{4a})—, —C(R³R^{3a})—O—, or —O—C(R³R^{3a})—, wherein in case —X¹— is a cyclic fragment, —X²— is a chemical bond, —C(R³R^{3a})—, —N(R³)— or —O—;
optionally, in case —X¹— is a cyclic fragment and —X²— is —C(R³R^{3a})—, the order of the —X¹— fragment and the —X²— fragment within the moiety of formula (a012) may be changed and the cyclic fragment is incorporated into the moiety of formula (a012) via two adjacent ring atoms;
—R¹, —R³ and —R⁴ are independently selected from the group consisting of —H, C₁₋₄ alkyl and —N(R⁵R^{5a}); —R^{1a}, —R², —R^{3a}, —R^{4a} and —R^{5a} are independently selected from the group consisting of —H, and C₁₋₄ alkyl;
—R⁵ is —C(O)R⁶;
—R⁶ is C₁₋₄ alkyl; and optionally, one of the pairs —R^{1a}/—R^{4a}, —R^{3a}/—R^{4a} or —R^{1a}/—R^{3a} form a chemical bond.

In certain embodiments -Tr and —Y₃— form together a functional group selected from the group consisting of

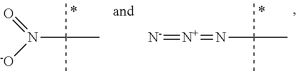

wherein the dashed line marked with the asterisk indicates attachment to —Ar—.

In certain embodiments -Tr is

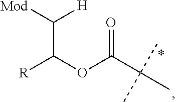

wherein -Mod and —R are as defined elsewhere within the section covering the exemplary multi-release linker herein.

### Suitably, -Mod is MeSO₂—.

In certain embodiments -Tr is selected from the group consisting of

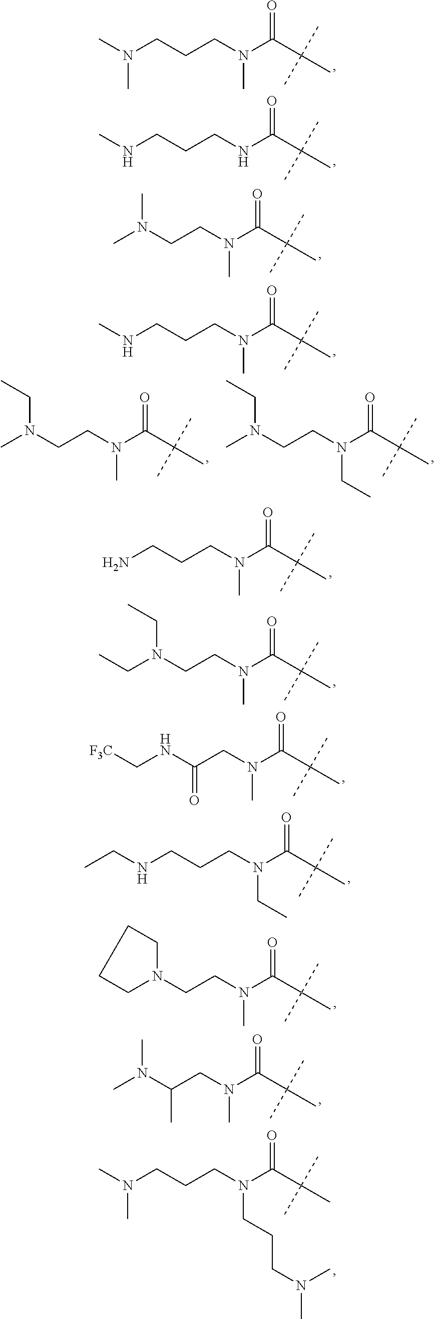

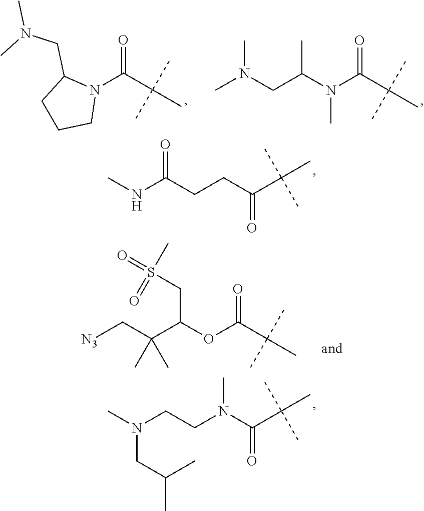

wherein the dashed line indicates attachment to —Y³— as defined elsewhere within the section covering the exemplary multi-release linker herein.

In certain embodiments -L^{1′}- is of formula (IIa1):

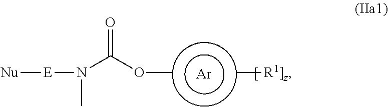

wherein
each —R¹ is independently selected from the group consisting of

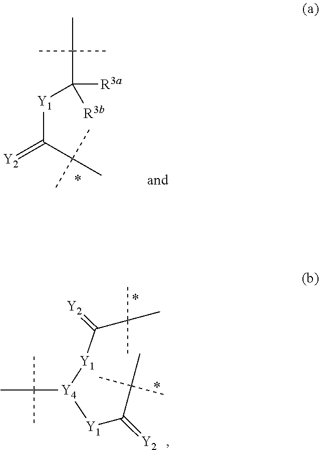

wherein
an unmarked dashed line indicates attachment to —Ar—;
a dashed line marked with an asterisk indicates attachment to -D-;
the total number of moieties -D- within the compound is ranging from 2 to 6; z is selected from the group consisting of 2 and 3;
each —Y₁— is —O—;
each ═Y₂ is ═O;
-Nu and -E are used as defined in formula (2aa), and
—Ar—, —Y₄—, —R^{3a} and —R^{3b} are used as defined in formula (2a); and
wherein -L^{1′}- is substituted with one moiety -L²-.

In certain embodiments all —R¹ of formula (IIa1) are of formula (a).

In certain embodiments all —R¹ of formula (IIa1) are of formula (b).

In certain embodiments -L^{1′}- is of formula (IIa2):

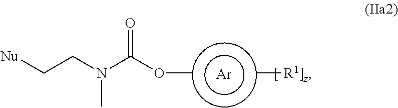

wherein
each —R¹ is independently selected from the group consisting of

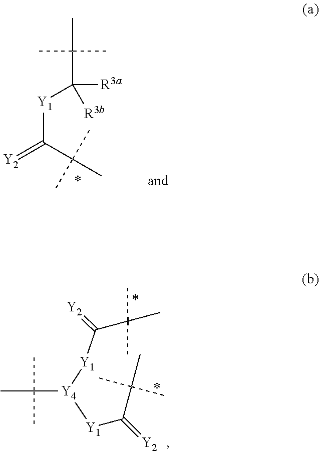

wherein
the unmarked dashed line indicates attachment to —Ar—;
a dashed line marked with an asterisk indicates attachment to -D-;
z is selected from the group consisting of 2 and 3;
the total number of moieties -D- within the compound ranges from 2 to 6;
-Nu is a secondary or tertiary amine functional group;
each —Y₁— is —O—;
each ═Y₂ is ═O;
—Ar—, —Y₄—, —R^{3a} and —R^{3b} are used as defined in formula (2a); and
wherein -L^{1′}- is substituted with one moiety -L²-.

In certain embodiments all —R¹ of formula (IIa2) are of formula (a).

In certain embodiments all —R¹ of formula (IIa2) are of formula (b).

In certain embodiments-L^{1′}- is selected from the group consisting of

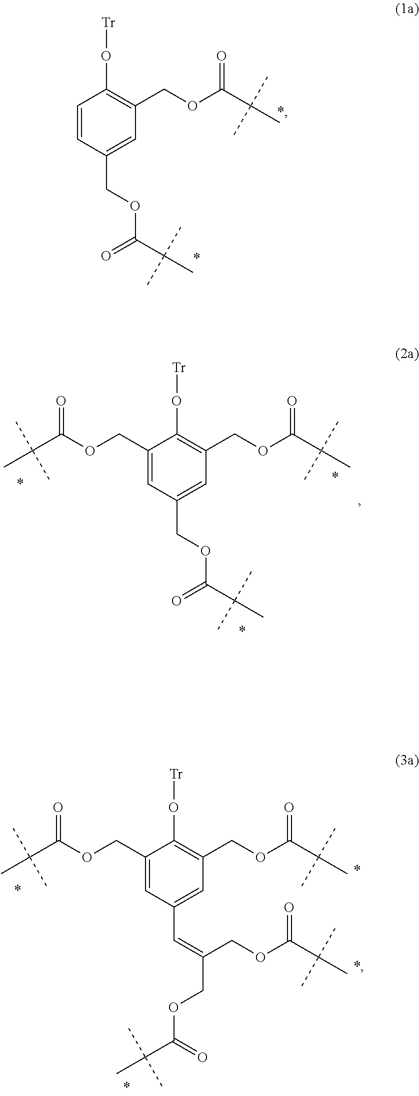

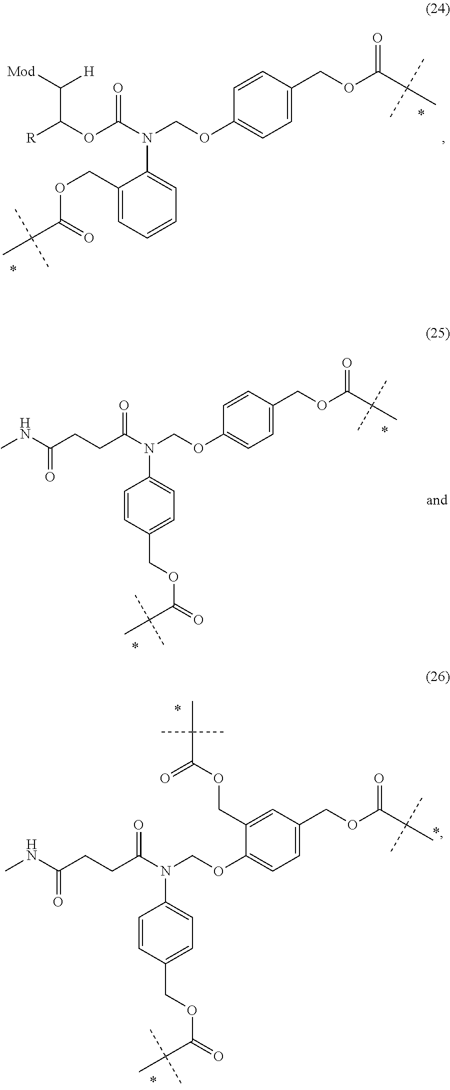

wherein
a dashed line marked with an asterisk indicates attachment to -D-;
—R and -Mod are used as described for formula (2ai); and
-Tr is selected from the group consisting of

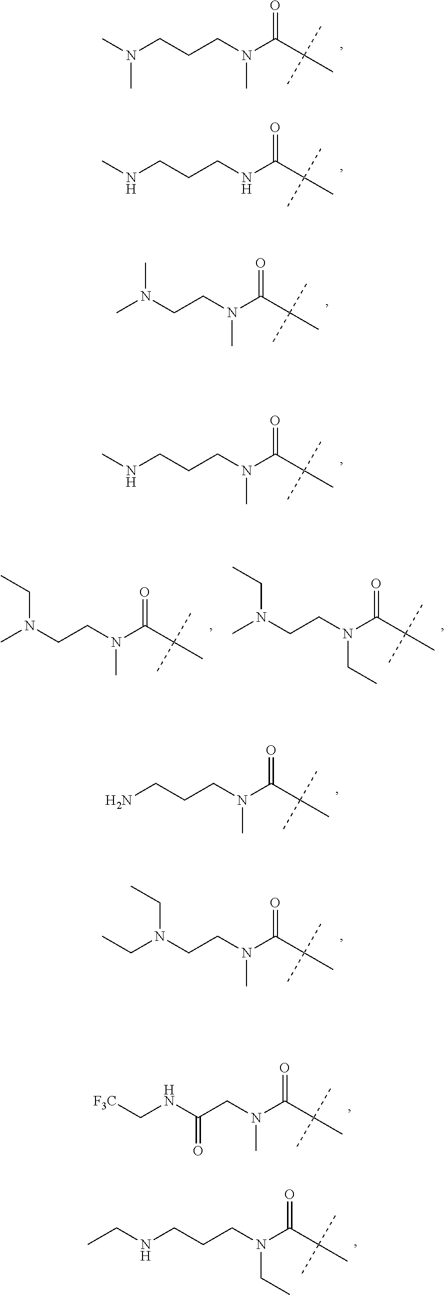

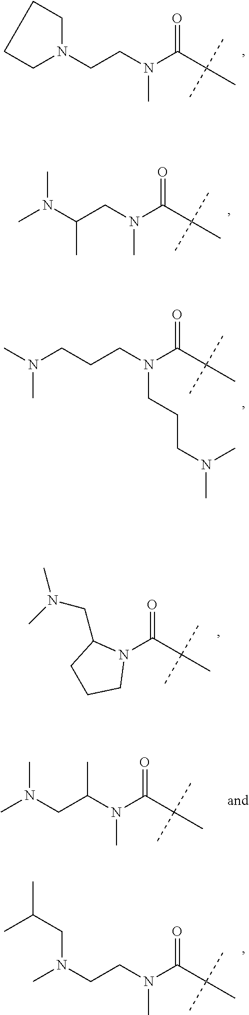

wherein the dashed line indicates attachment to the oxygen atom.

In certain embodiments -L^{1′}- is of formula (2b)

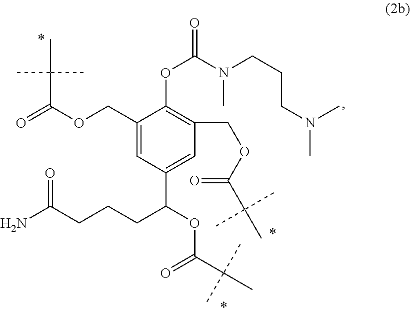

wherein
the dashed lines marked with the asterisk indicate attachment to -D-; and
-L^{1′}- is substituted with -L²-.

In certain embodiments the moiety -L^{1′}- is of formula (2b) and the dashed lines marked with the asterisk indicate attachment to semaglutide. In certain embodiments the moiety -L^{1′}- is of formula (2b) and the dashed lines marked with the asterisk indicate attachment to lixisenatide. In certain embodiments the moiety -L^{1′}- is of formula (2b) and the dashed lines marked with the asterisk indicate attachment to PEG-loxenatide. In certain embodiments the moiety -L^{1′}- is of formula (2b) and the dashed lines marked with the asterisk indicate attachment to liraglutide. In certain embodiments the moiety -L^{1′}- is of formula (2b) and the dashed lines marked with the asterisk indicate attachment to ecnoglutide. In certain embodiments the moiety -L^{1′}- is of formula (2b) and the dashed lines marked with the asterisk indicate attachment to GZR18. In certain embodiments the moiety -L^{1′}- is of formula (2b) and the dashed lines marked with the asterisk indicate attachment to GL0018. In certain embodiments the moiety -L^{1′}- is of formula (2b) and the dashed lines marked with the asterisk indicate attachment to tirzepatide. In certain embodiments the moiety -L^{1′}- is of formula (2b) and the dashed lines marked with the asterisk indicate attachment to cotadutide. In certain embodiments the moiety -L^{1′}- is of formula (2b) and the dashed lines marked with the asterisk indicate attachment to BI-456906. In certain embodiments the moiety -L^{1′}- is of formula (2b) and the dashed lines marked with the asterisk indicate attachment to pemvidutide. In certain embodiments the moiety -L^{1′}- is of formula (2b) and the dashed lines marked with the asterisk indicate attachment to mazdutide. In certain embodiments the moiety -L^{1′}- is of formula (2b) and the dashed lines marked with the asterisk indicate attachment to dapiglutide. In certain embodiments the moiety -L^{1′}- is of formula (2b) and the dashed lines marked with the asterisk indicate attachment to retatrutide. In certain embodiments the moiety -L^{1′}- is of formula (2b) and the dashed lines marked with the asterisk indicate attachment to the compound of SEQ ID NO:52. In certain embodiments the moiety -L^{1′}- is of formula (2b) and the dashed lines marked with the asterisk indicate attachment to the compound of SEQ ID NO:53.

In certain embodiments -L^{1′}- is of formula (2b-i)

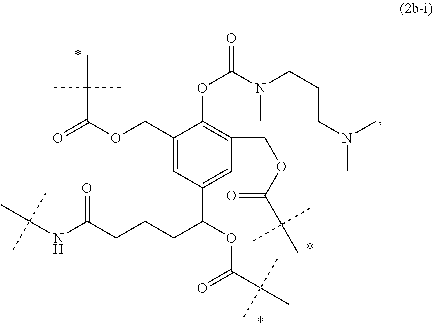

wherein
the dashed lines marked with the asterisk indicate attachment to -D-; and
the unmarked dashed line indicates attachment to -L²

In certain embodiments the moiety -L^{1′}- is of formula (2b-1) and the dashed lines marked with the asterisk indicate attachment to semaglutide. In certain embodiments the moiety -L^{1′}- is of formula (2b-1) and the dashed lines marked with the asterisk indicate attachment to lixisenatide. In certain embodiments the moiety -L^{1′}- is of formula (2b-1) and the dashed lines marked with the asterisk indicate attachment to PEG-loxenatide. In certain embodiments the moiety -L^{1′}- is of formula (2b-1) and the dashed lines marked with the asterisk indicate attachment to liraglutide. In certain embodiments the moiety -L^{1′}- is of formula (2b-1) and the dashed lines marked with the asterisk indicate attachment to ecnoglutide. In certain embodiments the moiety -L^{1′}- is of formula (2b-1) and the dashed lines marked with the asterisk indicate attachment to GZR18. In certain embodiments the moiety -L^{1′}- is of formula (2b-1) and the dashed lines marked with the asterisk indicate attachment to GL0018. In certain embodiments the moiety -L^{1′}- is of formula (2b-1) and the dashed lines marked with the asterisk indicate attachment to tirzepatide. In certain embodiments the moiety -L^{1′}- is of formula (2b-1) and the dashed lines marked with the asterisk indicate attachment to cotadutide. In certain embodiments the moiety -L^{1′}- is of formula (2b-1) and the dashed lines marked with the asterisk indicate attachment to BI-456906. In certain embodiments the moiety -L^{1′}- is of formula (2b-i) and the dashed lines marked with the asterisk indicate attachment to pemvidutide. In certain embodiments the moiety -L^{1′}- is of formula (2b-1) and the dashed lines marked with the asterisk indicate attachment to mazdutide. In certain embodiments the moiety -L^{1′}- is of formula (2b-i) and the dashed lines marked with the asterisk indicate attachment to dapiglutide. In certain embodiments the moiety -L^{1′}- is of formula (2b-1) and the dashed lines marked with the asterisk indicate attachment to retatrutide. In certain embodiments the moiety -L^{1′}- is of formula (2b-i) and the dashed lines marked with the asterisk indicate attachment to the compound of SEQ ID NO:52. In certain embodiments the moiety -L^{1′}- is of formula (2b-1) and the dashed lines marked with the asterisk indicate attachment to the compound of SEQ ID NO:53.

In certain embodiments -L²-L^{1′}- is of formula (2b-ii)

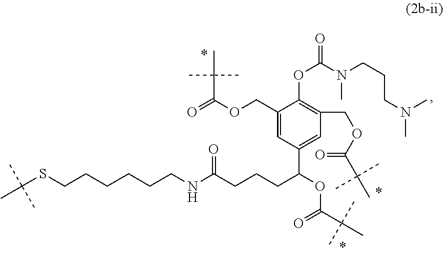

wherein
the dashed lines marked with the asterisk indicate attachment to -D-; and
the unmarked dashed line indicates attachment to the at least one polymeric moiety, to formula (I-ai), to formula (I-aii) or to formula (I-aiii).

In certain embodiments the unmarked dashed line in formula (2b-ii) indicates attachment to the at least one polymeric moiety. In certain embodiments the unmarked dashed line in formula (2b-ii) indicates attachment to formula (I-ai). In certain embodiments the unmarked dashed line in formula (2b-ii) indicates attachment to formula (I-aii). In certain embodiments the unmarked dashed line in formula (2b-ii) indicates attachment to formula (I-aiii).

In certain embodiments the moiety -L²-L^{1′}- is of formula (2b-ii) and the dashed lines marked with the asterisk indicate attachment to semaglutide, in particular attachment to the N-terminal amine functional group of semaglutide. In certain embodiments the moiety -L²-L^{1′}- is of formula (2b-ii) and the dashed lines marked with the asterisk indicate attachment to lixisenatide. In certain embodiments the moiety -L²-L^{1′}- is of formula (2b-ii) and the dashed lines marked with the asterisk indicate attachment to PEG-loxenatide. In certain embodiments the moiety -L²-L^{1′}- is of formula (2b-ii) and the dashed lines marked with the asterisk indicate attachment to liraglutide. In certain embodiments the moiety -L²-L^{1′}- is of formula (2b-ii) and the dashed lines marked with the asterisk indicate attachment to ecnoglutide. In certain embodiments the moiety -L²-L^{1′}- is of formula (2b-ii) and the dashed lines marked with the asterisk indicate attachment to GZR18. In certain embodiments the moiety -L²-L^{1′}- is of formula (2b-ii) and the dashed lines marked with the asterisk indicate attachment to GL0018. In certain embodiments the moiety -L²-L^{1′}- is of formula (2b-ii) and the dashed lines marked with the asterisk indicate attachment to tirzepatide. In certain embodiments the moiety -L²-L^{1′}- is of formula (2b-ii) and the dashed lines marked with the asterisk indicate attachment to cotadutide. In certain embodiments the moiety -L²-L^{1′}- is of formula (2b-ii) and the dashed lines marked with the asterisk indicate attachment to BI-456906. In certain embodiments the moiety -L²-L^{1′}- is of formula (2b-ii) and the dashed lines marked with the asterisk indicate attachment to pemvidutide. In certain embodiments the moiety -L²-L^{1′}- is of formula (2b-ii) and the dashed lines marked with the asterisk indicate attachment to mazdutide. In certain embodiments the moiety -L²-L^{1′}- is of formula (2b-ii) and the dashed lines marked with the asterisk indicate attachment to dapiglutide. In certain embodiments the moiety -L²-L^{1′}- is of formula (2b-ii) and the dashed lines marked with the asterisk indicate attachment to retatrutide. In certain embodiments the moiety -L²-L^{1′}- is of formula (2b-ii) and the dashed lines marked with the asterisk indicate attachment to the compound of SEQ ID NO:52. In certain embodiments the moiety -L²-L^{1′}- is of formula (2b-ii) and the dashed lines marked with the asterisk indicate attachment to the compound of SEQ ID NO:53.

In certain embodiments -L^{1′}- is of formula (2b′)

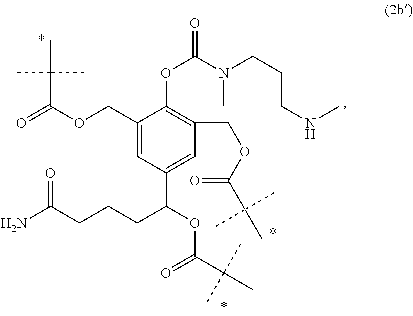

wherein
the dashed lines marked with the asterisk indicate attachment to -D-; and
-L^{1′}- is substituted with -L²-.

In certain embodiments the moiety -L^{1′}- is of formula (2b′) and the dashed lines marked with the asterisk indicate attachment to semaglutide, in particular attachment to the N-terminal amine functional group of semaglutide. In certain embodiments the moiety -L^{1′}- is of formula (2b′) and the dashed lines marked with the asterisk indicate attachment to lixisenatide. In certain embodiments the moiety -L^{1′}- is of formula (2b′) and the dashed lines marked with the asterisk indicate attachment to PEG-loxenatide. In certain embodiments the moiety -L^{1′}- is of formula (2b′) and the dashed lines marked with the asterisk indicate attachment to liraglutide. In certain embodiments the moiety -L^{1′}- is of formula (2b′) and the dashed lines marked with the asterisk indicate attachment to ecnoglutide. In certain embodiments the moiety -L^{1′}- is of formula (2b′) and the dashed lines marked with the asterisk indicate attachment to GZR18. In certain embodiments the moiety -L^{1′}- is of formula (2b′) and the dashed lines marked with the asterisk indicate attachment to GL0018. In certain embodiments the moiety -L^{1′}- is of formula (2b′) and the dashed lines marked with the asterisk indicate attachment to tirzepatide. In certain embodiments the moiety -L^{1′}- is of formula (2b′) and the dashed lines marked with the asterisk indicate attachment to cotadutide. In certain embodiments the moiety -L^{1′}- is of formula (2b′) and the dashed lines marked with the asterisk indicate attachment to BI-456906. In certain embodiments the moiety -L^{1′}- is of formula (2b′) and the dashed lines marked with the asterisk indicate attachment to pemvidutide. In certain embodiments the moiety -L^{1′}- is of formula (2b′) and the dashed lines marked with the asterisk indicate attachment to mazdutide. In certain embodiments the moiety -L^{1′}- is of formula (2b′) and the dashed lines marked with the asterisk indicate attachment to dapiglutide. In certain embodiments the moiety -L^{1′}- is of formula (2b′) and the dashed lines marked with the asterisk indicate attachment to retatrutide. In certain embodiments the moiety -L^{1′}- is of formula (2b′) and the dashed lines marked with the asterisk indicate attachment to the compound of SEQ ID NO:52. In certain embodiments the moiety -L^{1′}- is of formula (2b′) and the dashed lines marked with the asterisk indicate attachment to the compound of SEQ ID NO:53.

In certain embodiments -L^{1′}- is of formula (2b′-i)

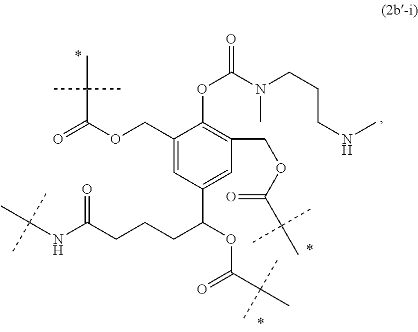

wherein
the dashed lines marked with the asterisk indicate attachment to -D-; and
the unmarked dashed line indicates attachment to -L²-.

In certain embodiments the moiety -L^{1′}- is of formula (2b′-i) and the dashed lines marked with the asterisk indicate attachment to semaglutide, in particular attachment to the N-terminal amine functional group of semaglutide. In certain embodiments the moiety -L^{1′}- is of formula (2b′-i) and the dashed lines marked with the asterisk indicate attachment to lixisenatide. In certain embodiments the moiety -L^{1′}- is of formula (2b′-i) and the dashed lines marked with the asterisk indicate attachment to PEG-loxenatide. In certain embodiments the moiety -L^{1′}- is of formula (2b′-i) and the dashed lines marked with the asterisk indicate attachment to liraglutide. In certain embodiments the moiety -L^{1′}- is of formula (2b′-i) and the dashed lines marked with the asterisk indicate attachment to ecnoglutide. In certain embodiments the moiety -L^{1′}- is of formula (2b′-i) and the dashed lines marked with the asterisk indicate attachment to GZR18. In certain embodiments the moiety -L^{1′}- is of formula (2b′-i) and the dashed lines marked with the asterisk indicate attachment to GL0018. In certain embodiments the moiety -L^{1′}- is of formula (2b′-i) and the dashed lines marked with the asterisk indicate attachment to tirzepatide. In certain embodiments the moiety -L^{1′}- is of formula (2b′-i) and the dashed lines marked with the asterisk indicate attachment to cotadutide. In certain embodiments the moiety -L^{1′}- is of formula (2b′-i) and the dashed lines marked with the asterisk indicate attachment to BI-456906. In certain embodiments the moiety -L^{1′}- is of formula (2b′-i) and the dashed lines marked with the asterisk indicate attachment to pemvidutide. In certain embodiments the moiety -L^{1′}- is of formula (2b′-i) and the dashed lines marked with the asterisk indicate attachment to mazdutide. In certain embodiments the moiety -L^{1′}- is of formula (2b′-i) and the dashed lines marked with the asterisk indicate attachment to dapiglutide. In certain embodiments the moiety -L^{1′}- is of formula (2b′-i) and the dashed lines marked with the asterisk indicate attachment to retatrutide. In certain embodiments the moiety -L^{1′}- is of formula (2b′-i) and the dashed lines marked with the asterisk indicate attachment to the compound of SEQ ID NO:52. In certain embodiments the moiety -L^{1′}- is of formula (2b′-i) and the dashed lines marked with the asterisk indicate attachment to the compound of SEQ ID NO:53.

In certain embodiments -L²-L^{1′}- is of formula (2b′-ii)

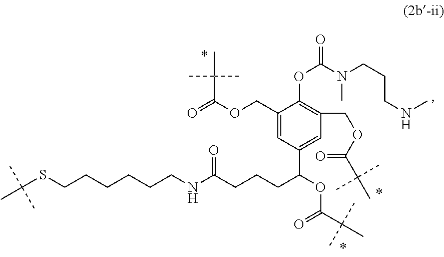

wherein
the dashed lines marked with the asterisk indicate attachment to -D-; and
the unmarked dashed line indicates attachment to the at least one polymeric moiety, to formula (I-ai), to formula (I-aii) or to formula (I-aiii).

In certain embodiments the unmarked dashed line in formula (2b′-ii) indicates attachment to the at least one polymeric moiety. In certain embodiments the unmarked dashed line in formula (2b′-ii) indicates attachment to formula (I-ai). In certain embodiments the unmarked dashed line in formula (2b′-ii) indicates attachment to formula (I-aii). In certain embodiments the unmarked dashed line in formula (2b′-ii) indicates attachment to formula (I-aiii).

In certain embodiments the moiety -L²-L^{1′}- is of formula (2b′-ii) and the dashed lines marked with the asterisk indicate attachment to semaglutide, in particular attachment to the N-terminal amine functional group of semaglutide. In certain embodiments the moiety -L²-L^{1′}- is of formula (2b′-ii) and the dashed lines marked with the asterisk indicate attachment to lixisenatide. In certain embodiments the moiety -L²-L^{1′}- is of formula (2b′-ii) and the dashed lines marked with the asterisk indicate attachment to PEG-loxenatide. In certain embodiments the moiety -L²-L^{1′}- is of formula (2b′-ii) and the dashed lines marked with the asterisk indicate attachment to liraglutide. In certain embodiments the moiety -L²-L^{1′}- is of formula (2b′-ii) and the dashed lines marked with the asterisk indicate attachment to ecnoglutide. In certain embodiments the moiety -L²-L^{1′}- is of formula (2b′-ii) and the dashed lines marked with the asterisk indicate attachment to GZR18. In certain embodiments the moiety -L²-L^{1′}- is of formula (2b′-ii) and the dashed lines marked with the asterisk indicate attachment to GL0018. In certain embodiments the moiety -L²-L^{1′}- is of formula (2b′-ii) and the dashed lines marked with the asterisk indicate attachment to tirzepatide. In certain embodiments the moiety -L²-L^{1′}- is of formula (2b′-ii) and the dashed lines marked with the asterisk indicate attachment to cotadutide. In certain embodiments the moiety -L²-L^{1′}- is of formula (2b′-ii) and the dashed lines marked with the asterisk indicate attachment to BI-456906. In certain embodiments the moiety -L²-L^{1′}- is of formula (2b′-ii) and the dashed lines marked with the asterisk indicate attachment to pemvidutide. In certain embodiments the moiety -L²-L^{1′}- is of formula (2b′-ii) and the dashed lines marked with the asterisk indicate attachment to mazdutide. In certain embodiments the moiety -L²-L^{1′}- is of formula (2b′-ii) and the dashed lines marked with the asterisk indicate attachment to dapiglutide. In certain embodiments the moiety -L²-L^{1′}- is of formula (2b′-ii) and the dashed lines marked with the asterisk indicate attachment to retatrutide. In certain embodiments the moiety -L²-L^{1′}- is of formula (2b′-ii) and the dashed lines marked with the asterisk indicate attachment to the compound of SEQ ID NO:52. In certain embodiments the moiety -L²-L^{1′}- is of formula (2b′-ii) and the dashed lines marked with the asterisk indicate attachment to the compound of SEQ ID NO:53.

In certain embodiments the compound is of formula (2c)

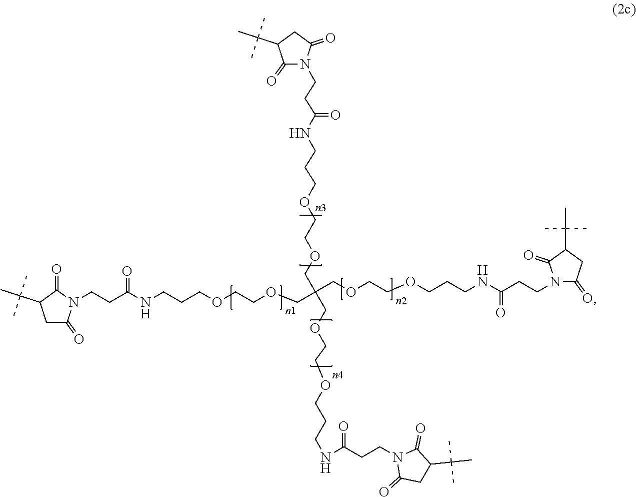

wherein
n1, n2, n3 and n4 are independently an integer ranging from about 11 to about 115; dashed lines indicate attachment to a moiety

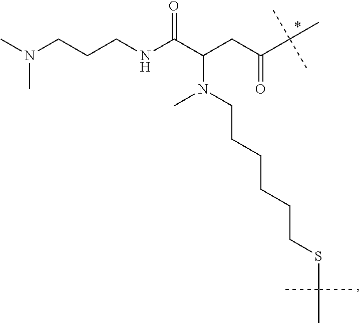

wherein
the unmarked dashed line indicates attachment to an unmarked dashed line of formula (2c); and
the dashed line marked with the asterisk indicates attachment to the N-terminal amine functional group of semaglutide.

In certain embodiments the compound is of formula (2c″)

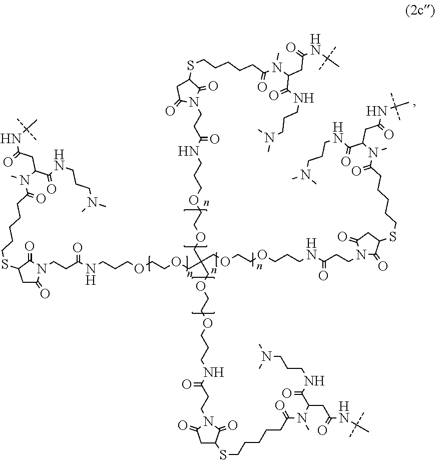

wherein
the dashed line indicates attachment to the N-terminal amine functional group of semaglutide; and
n1, n2, n3 and n4 are independently an integer ranging from about 11 to about 115.

In certain embodiments the compound is of formula (2c′)

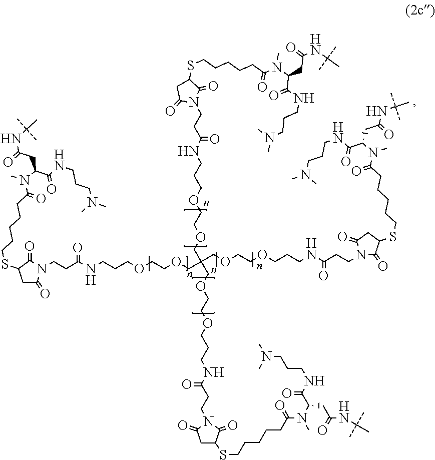

wherein
the dashed line indicates attachment to the N-terminal amine functional group of semaglutide; and
n1, n2, n3 and n4 are independently an integer ranging from about 11 to about 115.

In certain embodiments n1, n2, n3 and n4 of formula (2c), (2c′) and (2c″) are about 22. In certain embodiments n1, n2, n3 and n4 of formula (2c), (2c′) and (2c″) are about 23. In certain embodiments n1, n2, n3 and n4 of formula (2c), (2c′) and (2c″) are about 28. In certain embodiments n1, n2, n3 and n4 of formula (2c), (2c′) and (2c″) are about 34. In certain embodiments n1, n2, n3 and n4 of formula (2c), (2c′) and (2c″) are about 45. In certain embodiments n1, n2, n3 and n4 of formula (2c), (2c′) and (2c″) are about 56. In certain embodiments n1, n2, n3 and n4 of formula (2c), (2c′) and (2c″) are about 68. In certain embodiments n1, n2, n3 and n4 of formula (2c), (2c′) and (2c″) are about 80. In certain embodiments n1, n2, n3 and n4 of formula (2c), (2c′) and (2c″) are about 90. In certain embodiments n1, n2, n3 and n4 of formula (2c), (2c′) and (2c″) are about 100. In certain embodiments n1, n2, n3 and n4 of formula (2c), (2c′) and (2c″) are about 115.

In certain embodiments n1, n2, n3 and n4 of formula (2c), (2c′) and (2c″) are selected such that the total molecular weight of the moiety

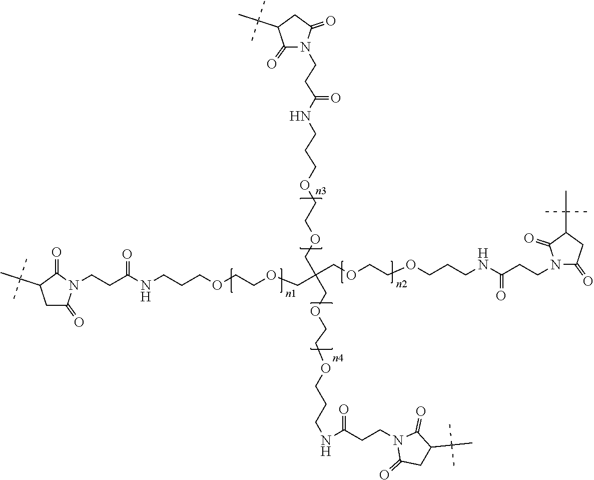

ranges from 7 to 13 kDa, such as from 8 to 12, from 9 to 11 kDa or is about 10 kDa. In certain embodiments n1, n2, n3 and n4 of formula (2c), (2c′) and (2c″) are selected such that the total molecular weight of this moiety ranges from 4 to 6 kDa or is about 5 kDa.

In certain embodiments the compound is of formula (2d)

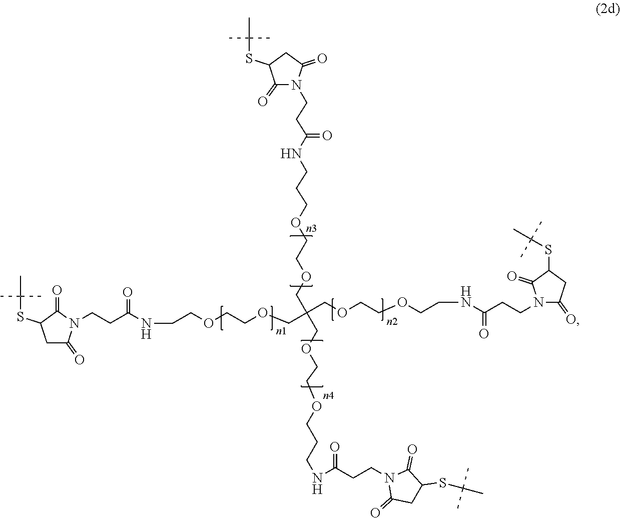

wherein
n1, n2, n3 and n4 are independently an integer ranging from about 11 to about 115; dashed lines indicate attachment to a moiety

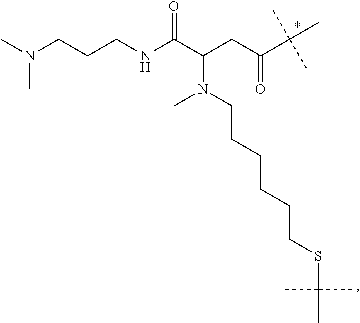

wherein
the unmarked dashed line indicates attachment to an unmarked dashed line of formula (2d); and
the dashed line marked with the asterisk indicates attachment to the N-terminal amine functional group of semaglutide.

In certain embodiments n1, n2, n3 and n4 of formula (2d) are about 22. In certain embodiments n1, n2, n3 and n4 of formula (2d) are about 23. In certain embodiments n1, n2, n3 and n4 of formula (2d) are about 34. In certain embodiments n1, n2, n3 and n4 of formula (2d) are about 45. In certain embodiments n1, n2, n3 and n4 of formula (2d) are about 56. In certain embodiments n1, n2, n3 and n4 of formula (2d) are about 68. In certain embodiments n1, n2, n3 and n4 of formula (2d) are about 80. In certain embodiments n1, n2, n3 and n4 of formula (2d) are about 90. In certain embodiments n1, n2, n3 and n4 of formula (2d) are about 100. In certain embodiments n1, n2, n3 and n4 of formula (2d) are about 115.

In certain embodiments n1, n2, n3 and n4 of formula (2d) are selected such that the total molecular weight of the moiety

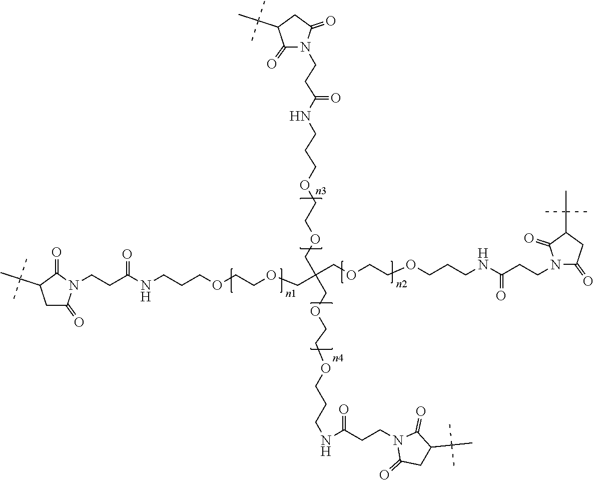

ranges from 7 to 13 kDa, such as from 8 to 12, from 9 to 11 kDa or is about 10 kDa.

In certain embodiments the compound is of formula (2e)

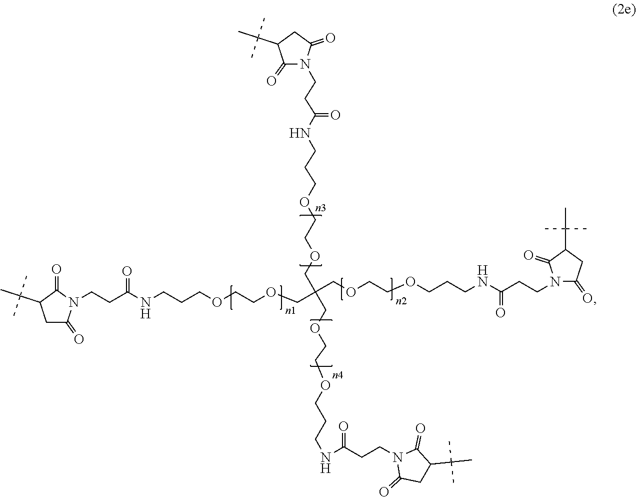

wherein
n1, n2, n3 and n4 are independently an integer ranging from about 11 to about 115; dashed lines indicate attachment to a moiety

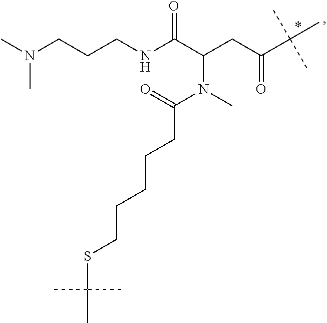

wherein
the unmarked dashed line indicates attachment to an unmarked dashed line of formula (2e); and
the dashed line marked with the asterisk indicates attachment to the N-terminal amine functional group of semaglutide.

In certain embodiments n1, n2, n3 and n4 of formula (2e) are about 22. In certain embodiments n1, n2, n3 and n4 of formula (2e) are about 23. In certain embodiments n1, n2, n3 and n4 of formula (2e) are about 34. In certain embodiments n1, n2, n3 and n4 of formula (2e) are about 45. In certain embodiments n1, n2, n3 and n4 of formula (2e) are about 56. In certain embodiments n1, n2, n3 and n4 of formula (2e) are about 68. In certain embodiments n1, n2, n3 and n4 of formula (2e) are about 80. In certain embodiments n1, n2, n3 and n4 of formula (2e) are about 90. In certain embodiments n1, n2, n3 and n4 of formula (2e) are about 100. In certain embodiments n1, n2, n3 and n4 of formula (2e) are about 115.

In certain embodiments n1, n2, n3 and n4 of formula (2e) are selected such that the total molecular weight of the moiety

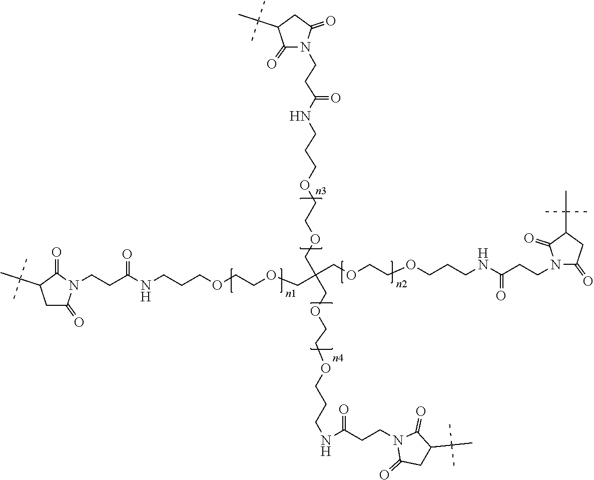

ranges from 7 to 13 kDa, such as from 8 to 12, from 9 to 11 kDa or is about 10 kDa. In certain embodiments the total molecular weight of this moiety is about 5 kDa.

In certain embodiments the compound is of formula (2f)

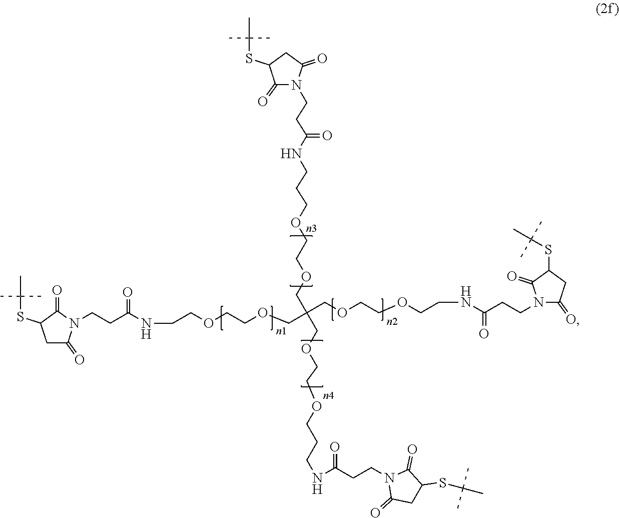

wherein
n1, n2, n3 and n4 are independently an integer ranging from about 11 to about 115; dashed lines indicate attachment to a moiety

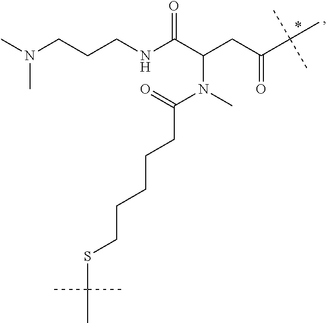

wherein
the unmarked dashed line indicates attachment to an unmarked dashed line of formula (2f); and
the dashed line marked with the asterisk indicates attachment to the N-terminal amine functional group of semaglutide.

In certain embodiments n1, n2, n3 and n4 of formula (2f) are about 22. In certain embodiments n1, n2, n3 and n4 of formula (2f) are about 23. In certain embodiments n1, n2, n3 and n4 of formula (2f) are about 34. In certain embodiments n1, n2, n3 and n4 of formula (2f) are about 45. In certain embodiments n1, n2, n3 and n4 of formula (2f) are about 56. In certain embodiments n1, n2, n3 and n4 of formula (2f) are about 68. In certain embodiments n1, n2, n3 and n4 of formula (2f) are about 80. In certain embodiments n1, n2, n3 and n4 of formula (2f) are about 90. In certain embodiments n1, n2, n3 and n4 of formula (2f) are about 100. In certain embodiments n1, n2, n3 and n4 of formula (2f) are about 115.

In certain embodiments n1, n2, n3 and n4 of formula (2f) are selected such that the total molecular weight of the moiety

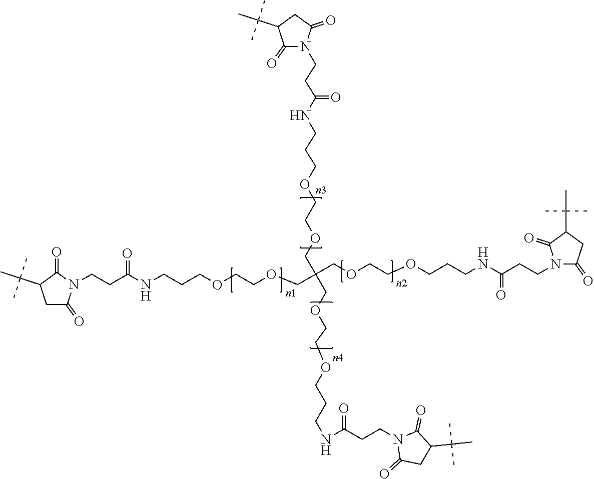

ranges from 7 to 13 kDa, such as from 8 to 12, from 9 to 11 kDa or is about 10 kDa. In certain embodiments the total molecular weight of this moiety is about 5 kDa.

In certain embodiments the compound is of formula (2g)

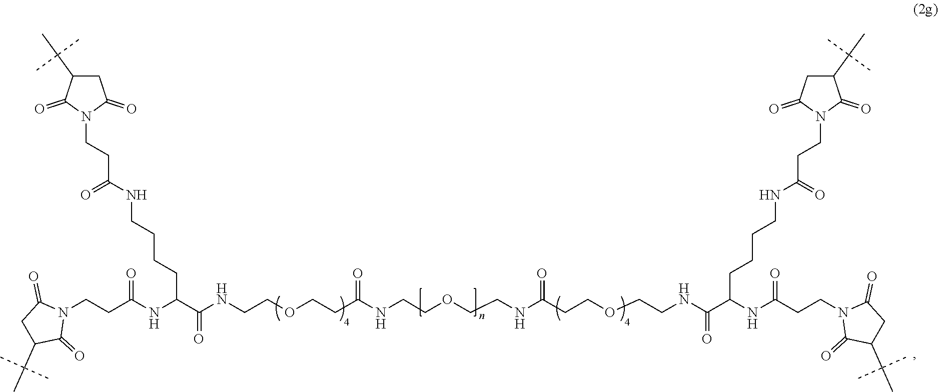

wherein
n is independently an integer ranging from about 22 to about 230;
dashed lines indicate attachment to a moiety

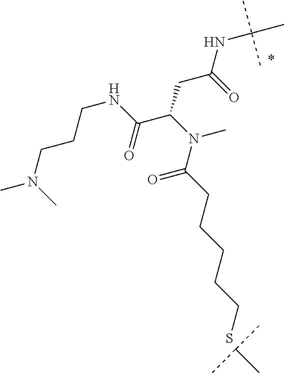

wherein
the unmarked dashed line indicates attachment to an unmarked dashed line of formula (2g); and
the dashed line marked with the asterisk indicates attachment to the N-terminal amine functional group of semaglutide.

In certain embodiments the compound is of formula (2g′)

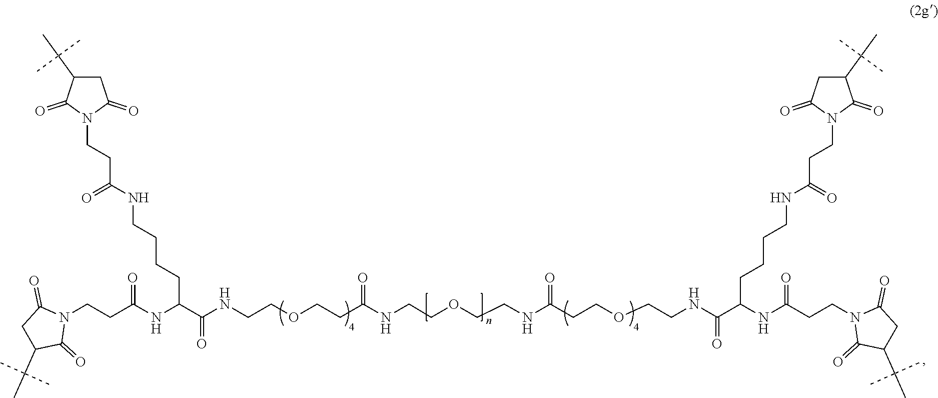

wherein
n is independently an integer ranging from about 22 to about 230;
dashed lines indicate attachment to a moiety

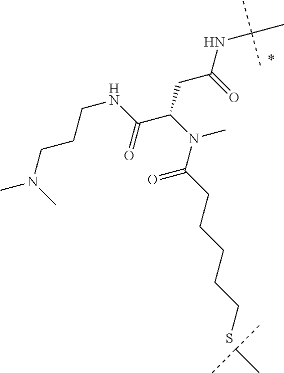

wherein
the unmarked dashed line indicates attachment to an unmarked dashed line of formula (2g′); and
the dashed line marked with the asterisk indicates attachment to the N-terminal amine functional group of semaglutide.

In certain embodiments n of formula (2g) and (2g′) is about 44. In certain embodiments n of formula (2g) and (2g′) is about 46. In certain embodiments n of formula (2c) is about 68. In certain embodiments n of formula (2g) and (2g′) is about 90. In certain embodiments n of formula (2g) and (2g′) is about 112. In certain embodiments n of formula (2g) and (2g′) is about 90. In certain embodiments n of formula (2g) and (2g′) is about 113. In certain embodiments n of formula (2g) and (2g′) is about 136. In certain embodiments n of formula (2g) and (2g′) is about 160. In certain embodiments n of formula (2g) and (2g′) is about 180.

In certain embodiments n of formula (2g) and (2g′) is about 200. In certain embodiments n of formula (2g) and (2g′) is about 230.

In certain embodiments n of formula (2g) is selected such that the total molecular weight of the moiety

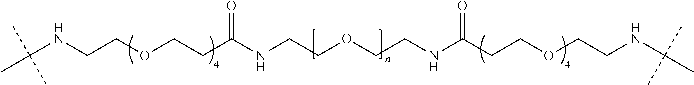

ranges from 3 to 7 kDa, such as from 4 to 6, from 4.5 to 5.5 kDa or is about 5 kDa.

In certain embodiments the compound is of formula (2h)

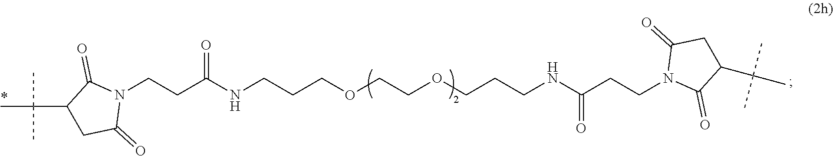

wherein
the dashed line marked with the asterisk indicates attachment to the dashed line of a moiety of formula (21)

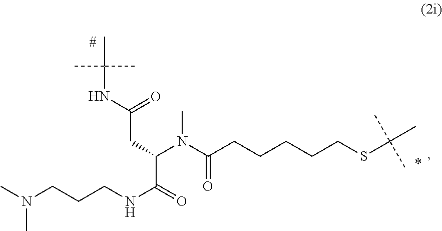

wherein
the dashed line marked with the # indicates attachment to the N-terminal amine functional group of semaglutide;
and wherein the unmarked dashed line of formula (2h) indicates attachment to the unmarked dashed line of formula (2j)

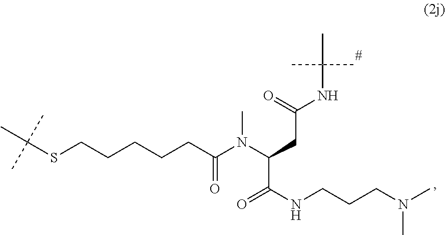

wherein
the dashed line marked with the # indicates attachment to the N-terminal amine functional group of semaglutide;
and wherein n is an integer ranging from about 22 to about 230.

In certain embodiments the compound is of formula (2h′)

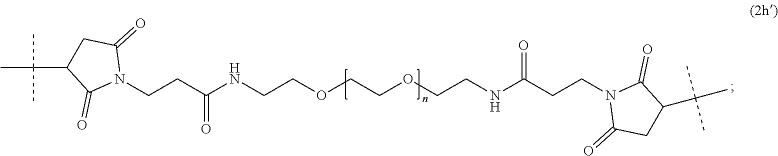

wherein
the dashed line marked with the asterisk indicates attachment to the dashed line marked with the asterisk of a moiety of formula (21)

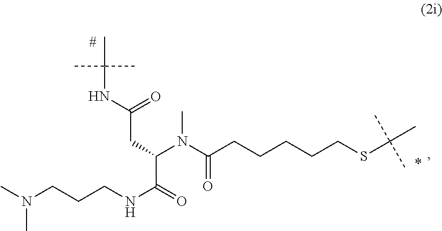

wherein
the dashed line marked with the # indicates attachment to the N-terminal amine functional group of semaglutide;
wherein the unmarked dashed line of formula (2h′) indicates attachment to the unmarked dashed line of formula (2j)

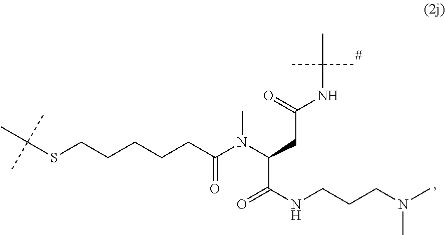

wherein
the dashed line marked with the # indicates attachment to the N-terminal amine functional group of semaglutide;
and wherein n is an integer ranging from about 22 to about 230.

In certain embodiments the compound is of formula (2 h″)

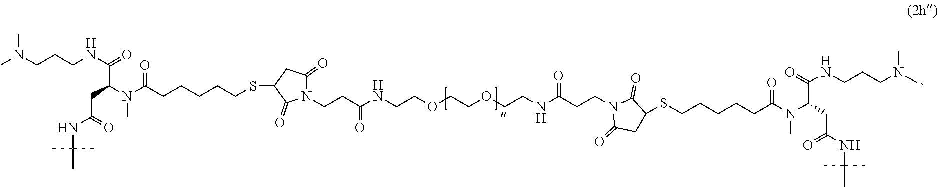

wherein
a dashed line indicates attachment to the N-terminal amine functional group of semaglutide.

In certain embodiments n of formula (2h′) is about 44. In certain embodiments n of formula (2h′) is about 46. In certain embodiments n of formula (2h′) is about 68. In certain embodiments n of formula (2h′) is about 90. In certain embodiments n of formula (2h′) is about 112. In certain embodiments n of formula (2h′) is about 113. In certain embodiments n of formula (2h′) is about 136. In certain embodiments n of formula (2h′) is about 160. In certain embodiments n of formula (2h′) is about 180. In certain embodiments n of formula (2h′) is about 200. In certain embodiments n of formula (2h′) is about 230.

Alternatively, the compounds of the present invention may also be a conjugate of a multilinker, as described in the following paragraphs.

In this case the compound of the present invention is a drug conjugate or a pharmaceutically acceptable salt thereof comprising a cleavable triggering moiety (-Tr), at least two drug moieties (-D) wherein each drug moiety comprises an albumin-binding moiety, one or more building blocks (-BB-), said building blocks being linked to one another in a linear or branching fashion, wherein upon cleavage of the triggering moiety said drug conjugate releases the least two drug moieties.

The drug conjugate or pharmaceutically acceptable salt thereof according to the present invention, wherein the drug conjugate comprises one or more building blocks (-BB-) of formula (I):

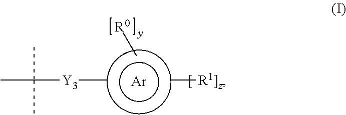

wherein the dashed line indicates the attachment to -Tr or to a carbon atom connected to =Y₂ provided that the drug conjugate comprises one -Tr;
each z is independently selected from the group consisting of 1, 2 and 3;
each y is independently selected from the group consisting of 0, 1, 2, 3, 4 and 5;
each —Y₃— is independently selected from the group consisting of —O—, —S— and —N(R⁶)—;
each —Ar— is a ring selected from the group consisting of monocyclic or bicyclic aryl and heteroaryl, provided that —Ar— is connected to —Y₃— and —R¹ via carbon atoms; wherein said monocyclic or bicyclic aryl and heteroaryl are optionally substituted with —R⁰, which are the same or different;
each —R¹ is independently selected from the group consisting of

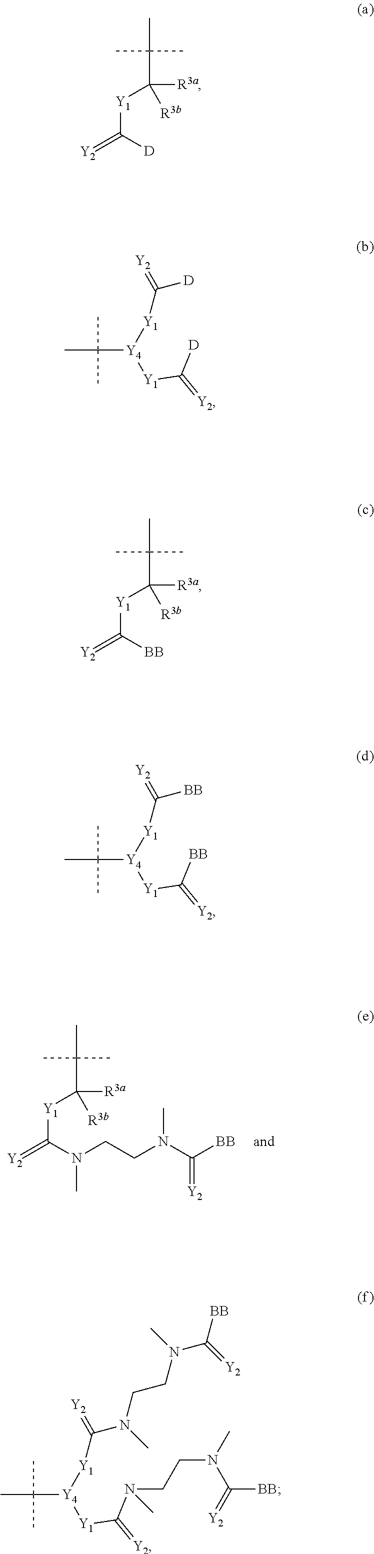

wherein the dashed line indicates the attachment to —Ar—;
each -D is a drug moiety comprising an albumin-binding moiety, wherein the total number of -Ds within the drug conjugate is ranging from 2 to 10;
each —Y₁— is independently selected from the group consisting of —O— and —S—;
each ═Y₂ is independently selected from the group consisting of ═O and ═S; each —Y₄— is

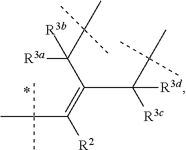

wherein the dashed line marked with the asterisk indicates the attachment to —Ar— and the unmarked dashed lines indicate the attachment to —Y₁—;
each —R⁰ is independently selected from the group consisting of —C(O)OH, -halogen, —NO₂, —CN, C₁₋₃₀ alkyl, C₂₋₃₀ alkenyl and C₂₋₃₀ alkynyl; wherein C₁₋₃₀ alkyl, C₂₋₃₀ alkenyl and C₂₋₃₀ alkynyl are optionally substituted with one or more —R⁴, which are the same or different; and wherein C₁₋₃₀ alkyl, C₂₋₃₀ alkenyl and C₂₋₃₀ alkynyl are optionally interrupted by one or more groups selected from the group consisting of -T-, —C(O)O—, —O—, —C(O)—, —C(O)N(R⁵)—, —S(O)₂N(R⁵)—, —S(O)N(R⁵)—, —S(O)₂—, —S(O)—, —N(R⁵)S(O)₂N(R^{5a})—, —S—, —N(R⁵)—, —OC(OR⁵)(R^{5a})—, —N(R⁵)C(O)N(R^{5a})— and —OC(O)N(R⁵)—;
each —R² is independently selected from the group consisting of —H, —C(O)OH, -halogen, —NO₂, —CN, C₁₋₃₀ alkyl, C₂₋₃₀ alkenyl and C₂₋₃₀ alkynyl; wherein C₁₋₃₀ alkyl, C₂₋₃₀ alkenyl and C₂₋₃₀ alkynyl are optionally substituted with one or more —R⁴, which are the same or different; and wherein C₁₋₃₀ alkyl, C₂₋₃₀ alkenyl and C₂₋₃₀ alkynyl are optionally interrupted by one or more groups selected from the group consisting of -T-, —C(O)O—, —O—, —C(O)—, —C(O)N(R⁵)—, —S(O)₂N(R⁵)—, —S(O)N(R⁵)—, —S(O)₂—, —S(O)—, —N(R⁵)S(O)₂N(R^{5a})—, —S—, —N(R⁵)—, —OC(OR⁵)(R^{5a})—, —N(R⁵)C(O)N(R^{5a})— and —OC(O)N(R⁵)—;
   each —R^{3a}, —R^{3b}, —R^{3c}, —R^{3d} are independently selected from the group consisting of —H, —C(O)OH, —F, —NO₂, —CN, C₁₋₃₀ alkyl, C₂₋₃₀ alkenyl and C₂₋₃₀ alkynyl; wherein C₁₋₃₀ alkyl, C₂₋₃₀ alkenyl and C₂₋₃₀ alkynyl are optionally substituted with one or more —R⁴, which are the same or different; and wherein C₁₋₃₀ alkyl, C₂₋₃₀ alkenyl and C₂₋₃₀ alkynyl are optionally interrupted by one or more groups selected from the group consisting of -T-, —C(O)O—, —O—, —C(O)—, —C(O)N(R⁵)—, —S(O)₂N(R⁵)—, —S(O)N(R⁵)—, —S(O)₂—, —S(O)—, —N(R⁵)S(O)₂N(R^{5a})—, —S—, —N(R⁵)—, —OC(OR⁵)(R^{5a})—, —N(R⁵)C(O)N(R^{5a})— and —OC(O)N(R⁵)—;
   each -T- is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each -T- is independently optionally substituted with one or more —R⁴, which are the same or different;
   wherein each —R⁴ is independently selected from the group consisting of —NO₂, —OCH₃, —CN, —N(R⁵)(R^{5a}), —OH, —C(O)OH, —N₃ and C₁₋₆ alkyl; wherein C₁₋₆ alkyl is optionally substituted with one or more halogen, which are the same or different;
   each —R⁵, —R^{5a} are independently selected from the group consisting of —H and C₁₋₆ alkyl; wherein C₁₋₆ alkyl is optionally substituted with one or more halogen, which are the same or different; and
   each —R⁶ is independently selected from the group consisting of —H, C₁₋₆ alkyl and —C(R^{3a})(R^{3b})—BB.

It is understood that within the expression “the attachment to a carbon atom connected to =Y₂″ refers to the carbon atom connected to =Y₂ in formulas (c), (d), (e) or (f).

It was found that the drug conjugate of the present invention was able to reversibly coordinate more than one albumin molecule or to at least two different domains of one albumin molecule. It was also found that the release half-life of the drug moiety that is released from the conjugate is higher than the half-life of an unmodified or unbound albumin comprising drug. Also, it was observed that subcutaneous administration of the drug conjugate may result in reduced absorption from the injection site compared to the subcutaneous administration of an unmodified or unbound albumin-comprising drug.

In certain embodiments, each —Y₃— is independently selected from the group consisting of —O— and —N(R⁶)—, wherein —R⁶ is described as elsewhere herein. In certain embodiments, each —Y₃— is —O—. In certain embodiments, each —Y₃— is —S—. In certain embodiments, each —Y₃— is —N(R⁶)—, wherein —R⁶ is described as elsewhere herein.

In certain embodiments, each —R¹ is independently selected from the group consisting of formulas (a), (b), (c) and (d). In certain embodiments, each —R¹ is independently selected from the group consisting of (a) and (b). In certain embodiments, each —R¹ is independently selected from the group consisting of (a) and (c). In certain embodiments, each —R¹ is independently selected from the group consisting of (a) and (d). In certain embodiments, each —R¹ is independently selected from the group consisting of (a) and (e). In certain embodiments, each —R¹ is independently selected from the group consisting of (a) and (f). In certain embodiments, each —R¹ is of formula (a). In certain embodiments, each —R¹ is of formula (b). In certain embodiments, each —R¹ is of formula (c). In certain embodiments, each —R¹ is of formula (d). In certain embodiments, each —R¹ is of formula (e). In certain embodiments, each —R¹ is of formula (f).

In certain embodiments, each z is independently selected from the group consisting of 1 and 2.

In certain embodiments, each z is 1. In certain embodiments, each z is 2. In certain embodiments, each z is 3.

In certain embodiments, each y is independently selected from the group consisting of 0, 1 and 2. In certain embodiments, each y is independently selected from the group consisting of 0 and 1. In certain embodiments, each y is 0. In certain embodiments, each y is 1. In certain embodiments, each y is 2. In certain embodiments, each y is 3. In certain embodiments, each y is 4. In certain embodiments, each y is 5.

The total number of -Ds within the drug conjugate according to the present invention is ranging from 2 to 10, such as from 2 to 8, such as from 2 to 6 or such as from 2 to 4. In certain embodiments, the total number of -Ds within the drug conjugate is 2. In certain embodiments, the total number of -Ds within the drug conjugate is 3. In certain embodiments, the total number of -Ds within the drug conjugate is 4. In certain embodiments, the total number of -Ds within the drug conjugate is 5. In certain embodiments, the total number of -Ds within the drug conjugate is 6. In certain embodiments, the total number of -Ds within the drug conjugate is 7. In certain embodiments, the total number of -Ds within the drug conjugate is 8. In certain embodiments, the total number of -Ds within the drug conjugate is 9. In certain embodiments, the total number of -Ds within the drug conjugate is 10.

In certain embodiments, each —Y₁— is —O—. In certain embodiments, each —Y₁— is —S—.

In certain embodiments, each ═Y₂ is ═O. In certain embodiments, each ═Y₂ is ═S.

In certain embodiments, each —Y₁— is —O— and each ═Y₂ is ═O.

In certain embodiments, each —Y₄— is

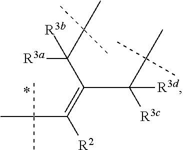

wherein the dashed line marked with the asterisk indicates the attachment to —Ar— and the unmarked dashed lines indicate the attachment to —Y₁— and —R², —R^{3a}, —R^{3b}, —R^{3c} and —R^{3d} are independently selected from the group consisting of —H, —C(O)OH, —F, —NO₂, —CN, C₁₋₆ alkyl, C₂₋₆ alkenyl and C₂₋₆ alkynyl.

In certain embodiments, each —Y₄— is

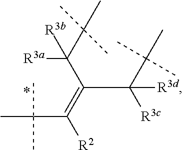

wherein the dashed line marked with the asterisk indicates the attachment to —Ar— and the unmarked dashed lines indicate the attachment to —Y₁— and —R², —R^{3a}, —R^{3b}, —R^{3c} and —R^{3d} are independently selected from the group consisting of —H and C₁₋₆ alkyl.

In certain embodiments, each —Y₄— is

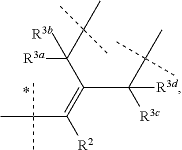

wherein the dashed line marked with the asterisk indicates the attachment to —Ar— and the unmarked dashed lines indicate the attachment to —Y₁— and —R², —R^{3a}, —R^{3b}, —R^{3c} and —R^{3d} are —H.

In certain embodiments, —Ar— is a monocyclic or bicyclic aryl ring, provided that —Ar— is connected to —Y₃— and —R¹ via carbon atoms, wherein said monocyclic or bicyclic aryl ring is optionally substituted with —R⁰ as described elsewhere herein, which are the same or different.

In certain embodiments, —Ar— is a monocyclic or bicyclic aryl ring, provided that —Ar— is connected to —Y₃— and —R¹ via carbon atoms, wherein said monocyclic or bicyclic aryl ring is not substituted with —R⁰. In certain embodiments, —Ar— is a monocyclic or bicyclic heteroaryl ring provided that —Ar— is connected to —Y₃— and —R¹ via carbon atoms, wherein said monocyclic or bicyclic heteroaryl ring is not substituted with —R⁰.

In certain embodiments, —Ar— is a monocyclic aryl ring, provided that —Ar— is connected to —Y₃— and —R¹ via carbon atoms, wherein said monocyclic aryl ring is optionally substituted with —R⁰ as described elsewhere herein, which are the same or different. In certain embodiments, —Ar— is a monocyclic aryl ring, provided that —Ar— is connected to —Y₃— and —R¹ via carbon atoms, wherein said monocyclic aryl ring is not substituted with —R⁰. Said monocyclic aryl ring may be selected from the group consisting of phenyl, pyridyl, pyrazinyl, pyridazinyl, pyrimidinyl and the like.

In certain embodiments, —Ar— is a bicyclic aryl ring, provided that —Ar— is connected to —Y₃— and —R¹ via carbon atoms, wherein said bicyclic aryl ring is optionally substituted with —R⁰ as described elsewhere herein, which are the same or different. In certain embodiments, —Ar— is a bicyclic aryl ring, provided that —Ar— is connected to —Y₃— and —R¹ via carbon atoms, wherein said bicyclic aryl ring is not substituted with —R⁰.

In certain embodiments, —Ar— is a monocyclic or bicyclic heteroaryl ring provided that —Ar— is connected to —Y₃— and —R¹ via carbon atoms, wherein said ring is optionally substituted with —R⁰ as described elsewhere herein, which are the same or different. In certain embodiments, —Ar— is a monocyclic heteroaryl ring, provided that —Ar— is connected to —Y₃— and —R¹ via carbon atoms, wherein said ring is not substituted with —R⁰.

It is understood that the cleavage mechanism within the drug conjugates of the present invention follows a 1,(4+2p′) elimination reaction, wherein p′ is 0, 1 or 2. That is to say that variables —Y³— and —R¹ are positioned on —Ar— in such as fashion to allow said elimination. This means that the linkage between -D and the rest of the drug conjugate may not be reversible per se, but is rendered reversible by said 1,(4+2p′) elimination reaction.

Generally this also means that the attachment of one or more —R¹ of formula (a), (b), (c), (d), (e) or (f) to —Ar— occurs so that said variables are at position(s) “1”, “1′” and/or “3” on the ring when considering that —Y³— is attached at position “0”. In other words, if for example —Ar— is a monocyclic or bicyclic aryl ring, then generally the attachment of one or more —R¹ of formula (a), (b), (c), (d), (e) or (f) occurs so that said variable(s) are in ortho or para position to —Y³—.

One example of how this concept may apply generally to a monocyclic aryl ring is shown below on

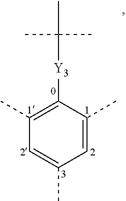

wherein the dashed lines at positions “1”, “1′” and/or “3” indicate the potential attachment points of —R¹ of formula (a), (b), (c), (d), (e) or (f). For simplicity, the optional variable “—R⁰” is not shown on the ring.

Also, another example of how this concept may apply generally to a bicyclic aryl ring is shown below on

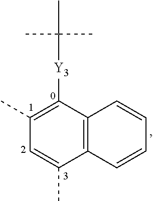

wherein the dashed lines at positions “1” and/or “3” indicate the potential attachment points of —R¹ of formula (a), (b), (c) or (d). For simplicity the optional variable “—R⁰” is not shown on the ring.

If z is 1, then —Ar— may be selected from the group consisting of

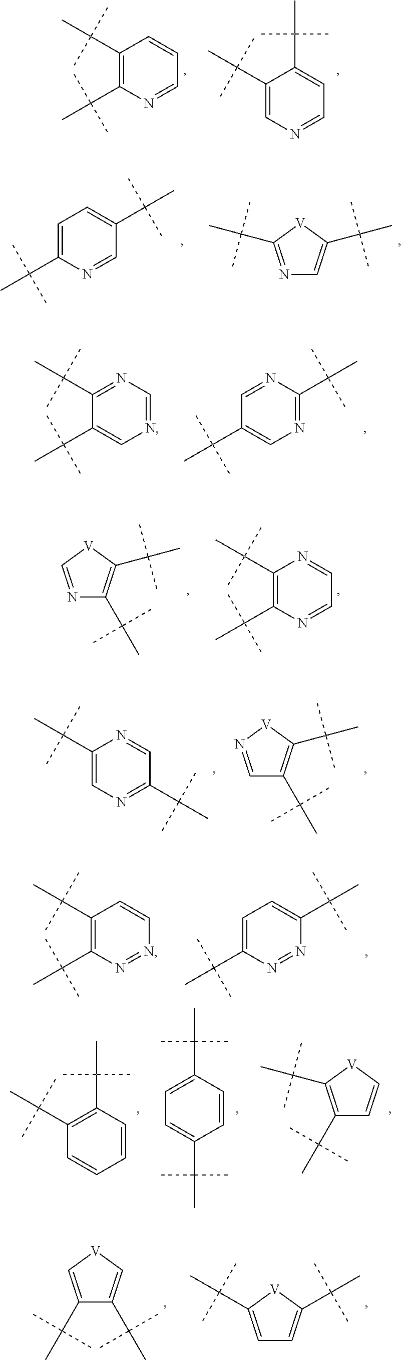

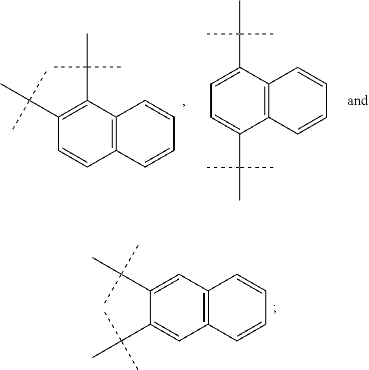

wherein one dashed line indicates the attachment to —Y³— and the other dashed line indicates the attachment to —R¹; and
each —V— is independently selected from the group consisting of —O—, —S— and —N(R⁵)—, wherein —R⁵ is selected from the group consisting of —H and C₁₋₆ alkyl.

If z is 2, then —Ar— may be selected from the group consisting of

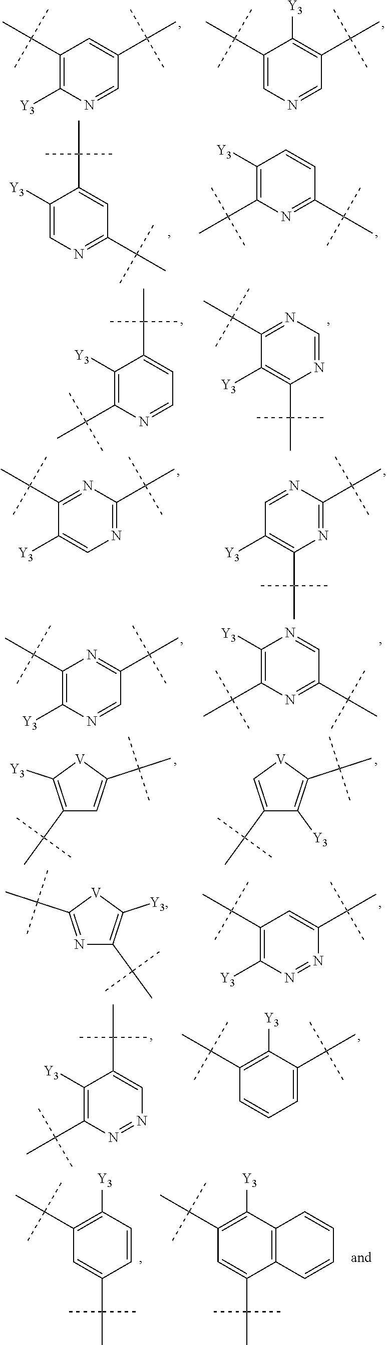

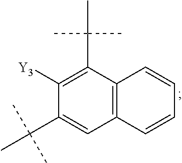

wherein the dashed lines indicate the attachment to —R¹; and
each —V— is independently selected from the group consisting of —O—, —S— and —N(R⁵)—, wherein —R⁵ is selected from the group consisting of —H and C₁₋₆ alkyl.

If z is 3, then —Ar— may be selected from the group consisting of

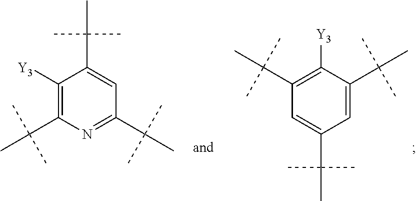

wherein the dashed lines indicate the attachment to —R¹.

Generally, —R⁰ can be attached at any position on the —Ar— ring that is not occupied by —R¹. In certain embodiments, each —R⁰ is independently selected from the group consisting of —C(O)OH, -halogen, —NO₂, —CN, C₁₋₂₀ alkyl, C₂₋₂₀ alkenyl and C₂₋₂₀ alkynyl; wherein C₁₋₂₀ alkyl, C₂₋₂₀ alkenyl and C₂₋₂₀ alkynyl are optionally substituted with one or more —R⁴, which are the same or different; and wherein C₁₋₂₀ alkyl, C₂₋₂₀ alkenyl and C₂₋₂₀ alkynyl are optionally interrupted by one or more groups selected from the group consisting of -T-, —C(O)O—, —O—, —C(O)—, —C(O)N(R⁵)—, —S(O)₂N(R⁵)—, —S(O)N(R⁵)—, —S(O)₂—, —S(O)—, —N(R⁵)S(O)₂N(R^{5a})—, —S—, —N(R⁵)—, —OC(OR⁵)(R^{5a})—, —N(R⁵)C(O)N(R^{5a})— and —OC(O)N(R⁵)—, wherein -T-, —R⁴, —R⁵ and —R^{5a} are as described elsewhere herein.

In certain embodiments, each —R⁰ is independently selected from the group consisting of —C(O)OH, -halogen, —NO₂, —CN, CI-10 alkyl, C₂₋₁₀ alkenyl and C₂₋₁₀ alkynyl; wherein C₁₋₁₀ alkyl, C₂₋₁₀ alkenyl and C₂₋₁₀ alkynyl are optionally substituted with one or more —R⁴, which are the same or different; and wherein C₁₋₁₀ alkyl, C₂₋₁₀ alkenyl and C₂₋₁₀ alkynyl are optionally interrupted by one or more groups selected from the group consisting of -T-, —C(O)O—, —O—, —C(O)—, —C(O)N(R⁵)—, —S(O)₂N(R⁵)—, —S(O)N(R⁵)—, —S(O)₂—, —S(O)—, —N(R⁵)S(O)₂N(R^{5a})—, —S—, —N(R⁵)—, —OC(OR⁵)(R^{5a})—, —N(R⁵)C(O)N(R^{5a})— and —OC(O)N(R⁵)—, wherein -T-, —R⁴, —R⁵ and —R^{5a} are as described elsewhere herein.

In certain embodiments, each —R⁰ is independently selected from the group consisting of —C(O)OH, -halogen, —NO₂, —CN, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R⁴, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R⁵)—, —S(O)₂N(R⁵)—, —S(O)N(R⁵)—, —S(O)₂—, —S(O)—, —N(R⁵)S(O)₂N(R^{5a})—, —S—, —N(R⁵)—, —OC(OR⁵)(R^{5a})—, —N(R⁵)C(O)N(R^{5a})— and —OC(O)N(R⁵)—, wherein -T-, —R⁴, —R⁵ and —R^{5a} are as described elsewhere herein.

In certain embodiments, each R⁰ is —C(O)OH. In certain embodiments, each —R⁰ is -halogen. In certain embodiments, each —R⁰ is —NO₂. In certain embodiments, each —R⁰ is —CN. In certain embodiments, each —R⁰ is C₁₋₆ alkyl. In certain embodiments, each —R⁰ is —CH₃. In certain embodiments, all —R⁰ are the same.

In certain embodiments, each —R⁰ is

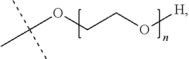

wherein the dashed line indicates the attachment to the carbon atom; and n ranges from 1 to 12, such as from 1 to 10, such as from 1 to 8 or such as from 1 to 6.

In certain embodiments, each —R² is independently selected from the group consisting of —H, —C(O)OH, -halogen, —NO₂, —CN, C₁₋₂₀ alkyl, C₂₋₂₀ alkenyl and C₂₋₂₀ alkynyl; wherein C₁₋₂₀ alkyl, C₂₋₂₀ alkenyl and C₂₋₂₀ alkynyl are optionally substituted with one or more —R⁴, which are the same or different; and wherein C₁₋₂₀ alkyl, C₂₋₂₀ alkenyl and C₂₋₂₀ alkynyl are optionally interrupted by one or more groups selected from the group consisting of -T-, —C(O)O—, —O—, —C(O)—, —C(O)N(R⁵)—, —S(O)₂N(R⁵)—, —S(O)N(R⁵)—, —S(O)₂—, —S(O)—, —N(R⁵)S(O)₂N(R^{5a})—, —S—, —N(R⁵)—, —OC(OR⁵)(R^{5a})—, —N(R⁵)C(O)N(R^{5a})— and —OC(O)N(R⁵)—, wherein -T-, —R⁴, —R⁵ and —R^{5a} are as described elsewhere herein.

In certain embodiments, each —R² is independently selected from the group consisting of —H, —C(O)OH, -halogen, —NO₂, —CN, C₁₋₁₀ alkyl, C₂₋₁₀ alkenyl and C₂₋₁₀ alkynyl; wherein C₁₋₁₀ alkyl, C₂₋₁₀ alkenyl and C₂₋₁₀ alkynyl are optionally substituted with one or more —R⁴, which are the same or different; and wherein C₁₋₁₀ alkyl, C₂₋₁₀ alkenyl and C₂₋₁₀ alkynyl are optionally interrupted by one or more groups selected from the group consisting of -T-, —C(O)O—, —O—, —C(O)—, —C(O)N(R⁵)—, —S(O)₂N(R⁵)—, —S(O)N(R⁵)—, —S(O)₂—, —S(O)—, —N(R⁵)S(O)₂N(R^{5a})—, —S—, —N(R⁵)—, —OC(OR⁵)(R^{5a})—, —N(R⁵)C(O)N(R^{5a})— and —OC(O)N(R⁵)—, wherein -T-, —R⁴, —R⁵ and —R^{5a} are as described elsewhere herein.

In certain embodiments, each —R² is independently selected from the group consisting of —H, —C(O)OH, -halogen, —NO₂, —CN, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R⁴, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R⁵)—, —S(O)₂N(R⁵)—, —S(O)N(R⁵)—, —S(O)₂—, —S(O)—, —N(R⁵)S(O)₂N(R^{5a})—, —S—, —N(R⁵)—, —OC(OR⁵)(R^{5a})—, —N(R⁵)C(O)N(R^{5a})— and —OC(O)N(R⁵)—, wherein -T-, —R⁴, —R⁵ and —R^{5a} are as described elsewhere herein.

In certain embodiments, each —R² is —H. In certain embodiments, each —R² is —C(O)OH. In certain embodiments, each —R² is -halogen. In certain embodiments, each —R² is —NO₂. In certain embodiments, each —R² is —CN. In certain embodiments, each —R² is C₁₋₆ alkyl. In certain embodiments, each —R² is —CH₃.

In certain embodiments, each —R² is

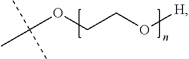

wherein the dashed line indicates the attachment to the carbon atom; and n ranges from 1 to 12, such as from 1 to 10, such as from 1 to 8 or such as from 1 to 6.

In certain embodiments, each —R^{3a}, R^{3b}, —R^{3c}, —R^{3d} are independently selected from the group consisting of —H, —C(O)OH, —F, —NO₂, —CN, C₁₋₂₀ alkyl, C₂₋₂₀ alkenyl and C₂₋₂₀ alkynyl; wherein C₁₋₂₀ alkyl, C₂₋₂₀ alkenyl and C₂₋₂₀ alkynyl are optionally substituted with one or more —R⁴, which are the same or different; and wherein C₁₋₂₀ alkyl, C₂₋₂₀ alkenyl and C₂₋₂₀ alkynyl are optionally interrupted by one or more groups selected from the group consisting of -T-, —C(O)O—, —O—, —C(O)—, —C(O)N(R⁵)—, —S(O)₂N(R⁵)—, —S(O)N(R⁵)—, —S(O)₂—, —S(O)—, —N(R⁵)S(O)₂N(R^{5a})—, —S—, —N(R⁵)—, —OC(OR⁵)(R^{5a})—, —N(R⁵)C(O)N(R^{5a})— and —OC(O)N(R⁵)—, wherein —R⁵ and —R^{5a} are as described elsewhere herein.

In certain embodiments, each —R^{3a}, R^{3b}, —R^{3c}, —R^{3d} are independently selected from the group consisting of —H, —C(O)OH, —F, —NO₂, —CN, C₁₋₁₀ alkyl, C₂₋₁₀ alkenyl and C₂₋₁₀ alkynyl; wherein C₁₋₁₀ alkyl, C₂₋₁₀ alkenyl and C₂₋₁₀ alkynyl are optionally substituted with one or more —R⁴, which are the same or different; and wherein C₁₋₁₀ alkyl, C₂₋₁₀ alkenyl and C₂₋₁₀ alkynyl are optionally interrupted by one or more groups selected from the group consisting of -T-, —C(O)O—, —O—, —C(O)—, —C(O)N(R⁵)—, —S(O)₂N(R⁵)—, —S(O)N(R⁵)—, —S(O)₂—, —S(O)—, —N(R⁵)S(O)₂N(R^{5a})—, —S—, —N(RS)—, —OC(OR⁵)(R^{5a})—, —N(R⁵)C(O)N(R^{5a})— and —OC(O)N(R⁵)—, wherein —R⁵ and —R^{5a} are as described elsewhere herein.

In certain embodiments, each —R^{3a}, —R^{3b}, —R^{3c} or —R^{3d} is independently

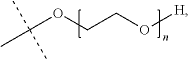

wherein the dashed line indicates the attachment to the carbon atom; and n ranges from 1 to 12, such as from 1 to 10, such as from 1 to 8 or such as from 1 to 6.

In certain embodiments, n is selected from the group consisting of 1, 2, 3, 4, 5 and 6. In certain embodiments, n is 1. In certain embodiments, n is 2. In certain embodiments, n is 3. In certain embodiments, n is 4. In certain embodiments, n is 5. In certain embodiments, n is 6.

In certain embodiments, all —R^{3a}, —R^{3b}, —R^{3c} and —R^{3d} are —H.

In certain embodiments, each -T- is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments, each -T- is phenyl. In certain embodiments, each -T- is naphthyl. In certain embodiments, each -T- is indenyl. In certain embodiments, each -T- is indanyl. In certain embodiments, each -T- is tetralinyl. In certain embodiments, each -T- is C₃₋₁₀ cycloalkyl. In certain embodiments, each -T- is 3- to 10-membered heterocyclyl. In certain embodiments, each -T- is 8- to 11-membered heterobicyclyl.

In certain embodiments, each —R⁴ is independently selected from the group consisting of —NO₂, —OCH₃, —CN, —N(R⁵)(R^{5a}), —OH, —C(O)OH and C₁₋₆ alkyl, wherein —R⁵ and —R^{5a} are as described elsewhere herein. In certain embodiments, each —R⁴ is independently selected from the group consisting of —NO₂, —OCH₃, —OH and C₁₋₆ alkyl.

In certain embodiments, each —R⁴ is —NO₂. In certain embodiments, each —R⁴ is —OCH₃. In certain embodiments, each —R⁴ is —CN. In certain embodiments, each —R⁴ is —N₃. In certain embodiments, each —R⁴ is —N(R⁵)(R^{5a}). In certain embodiments, each —R⁴ is —OH. In certain embodiments, each —R⁴ is —C(O)OH. In certain embodiments, each —R⁴ is C₁₋₆ alkyl.

In certain embodiments, each —R⁵ is —H. In certain embodiments, each —R⁵ is C₁₋₆ alkyl.

In certain embodiments, each —R^{5a} is —H. In certain embodiments, each —R^{5a} is C₁₋₆ alkyl. In certain embodiments, both —R⁵ and —R^{1a} are —H.

In certain embodiments, each —R⁶ is independently selected from the group consisting of —H and C₁₋₆ alkyl. In certain embodiments, each —R⁶ is —H. In certain embodiments, each —R⁶ is C₁₋₆ alkyl. In certain embodiments, each —R⁶ is —C(R^{3a})(R^{3b})—BB, wherein —R^{3a}, —R^{3b} and —BB are as described elsewhere herein.

The cleavable triggering moiety, -Tr may be selected from the group consisting of

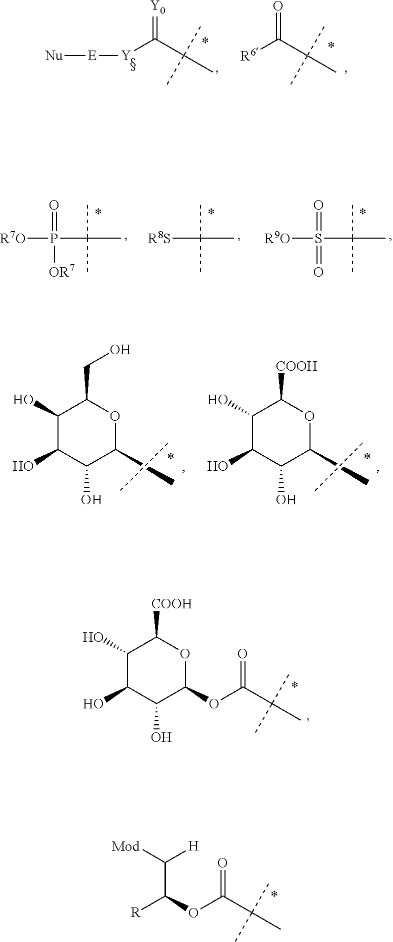

and
a peptidyl moiety;
   wherein the dashed line marked with an asterisk indicates the attachment to —Y³—;
   -Nu is a nucleophile; —Y_{§} — is selected from the group consisting of —O—, —C(R¹⁰)(R^{10a})—, —N(R⁶)— and —S—;
   ═Y₀ is selected from the group consisting of ═O, ═S and ═N(R⁵);
   -E- is selected from the group consisting of C₁₋₆ alkyl, C₂₋₆ alkenyl, C₂₋₆ alkynyl and
   -Q-; wherein C₁₋₆ alkyl, C₂₋₆ alkenyl, C₂₋₆ alkynyl are optionally substituted with one or more —R¹¹, which are the same or different; and wherein C₁₋₆ alkyl, C₂₋₆ alkenyl, C₂₋₆ alkynyl are optionally interrupted by one or more groups selected from the group consisting of -Q-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)R^{12a}—, —N(R¹²)C(O)N(R^{12a})— and —OC(O)N(R¹²)—;
   —R is selected from the group consisting of C₁₋₆ alkyl, C₂₋₆ alkenyl, C₂₋₆ alkynyl and -Q; wherein C₁₋₆ alkyl, C₂₋₆ alkenyl, C₂₋₆ alkynyl are optionally substituted with one or more —R¹¹, which are the same or different; and wherein C₁₋₆ alkyl, C₂₋₆ alkenyl, C₂₋₆ alkynyl are optionally interrupted by one or more groups selected from the group consisting of -Q-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)R^{12a}—, —N(R¹²)C(O)N(R^{12a})— and —OC(O)N(R¹²)—;
   -Mod is selected from the group consisting of CF₃PhSO₂—, ClPhSO₂—, PhSO₂—, MePhSO₂—, MeOPhSO₂—, 2,4,6-Me₃PhSO₂—, MeSO₂—, O(CH₂CH₂)₂N—SO₂—, CN— and Et₂NSO₂—;
   —R⁶ is independently selected from the group consisting of —H, C₁₋₆ alkyl and —C(R^{3a})(R^{3b})—BB, wherein —R^{3a}, —R^{3b} and —BB are as described elsewhere herein;
   —R^{5′}, —R^{6′}, —R⁷, —R⁸, —R⁹, —R¹⁰, —R^{10a} are independently selected from the group consisting of —H, C₁₋₂₀ alkyl, C₂₋₂₀ alkenyl, C₂₋₂₀ alkynyl and -Q; wherein C₁₋₂₀ alkyl, C₂₋₂₀ alkenyl and C₂₋₂₀ alkynyl are optionally substituted with one or more —R¹¹, which are the same or different; and wherein C₁₋₂₀ alkyl, C₂₋₂₀ alkenyl and C₂₋₂₀ alkynyl are optionally interrupted by one or more groups selected from the group consisting of -Q-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)R^{12a}—, —N(R¹²)C(O)N(R^{12a})— and —OC(O)N(R¹²)—;
      each Q is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each Q is independently optionally substituted with one or more —R¹¹, which are the same or different; and
      —R¹¹ is selected from the group consisting of C₁₋₆ alkyl and —N₃, wherein C₁₋₆ alkyl is optionally substituted with one or more halogen, which are the same or different; and
      —R¹² and —R^{12a} are independently selected from the group consisting of —H and C₁₋₆ alkyl; wherein C₁₋₆ alkyl is optionally substituted with one or more halogen, which are the same or different.

-Nu is a nucleophile that may be selected from the group consisting of primary, secondary, or tertiary amine and amide. In certain embodiments, -Nu is selected from the group consisting of secondary and tertiary amine. In certain embodiments, -Nu is a primary amine. In certain embodiments, -Nu is a secondary amine. In certain embodiments, -Nu is a tertiary amine. In certain embodiments, -Nu is an amide.

In certain embodiments, —Y_{§} — is selected from the group consisting of —O—, —C(R¹⁰)(R^{10a})— and —N(R⁶)—, wherein —R¹⁰, —R^{10a} and —R⁶ are as defined elsewhere herein. In certain embodiments, —Y_{§} — is —O—. In certain embodiments, —Y_{§} — is —C(R¹⁰)(R^{10a})—, wherein —R¹⁰ and —R^{10a} are as defined elsewhere herein. In certain embodiments, —Y_{§} — is —N(R⁶)—, wherein —R⁶ is as defined elsewhere herein.

In certain embodiments, ═Y₀ is selected from the group consisting of ═O and ═S. In certain embodiments, ═Y₀ is ═O. In certain embodiments, ═Y₀ is ═S. In certain embodiments, ═Y₀ is ═N(R′), wherein —R⁵ is as defined elsewhere herein.

In certain embodiments, -E- is selected from the group consisting of C₁₋₆ alkyl, C₂₋₆ alkenyl and C₂₋₆ alkynyl. In certain embodiments, -E- is C₁₋₆ alkyl. In certain embodiments, -E- is C₂₋₆ alkenyl. In certain embodiments, -E- is C₂₋₆ alkynyl. In certain embodiments, -E- is -Q-.

In certain embodiments, Q is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments, Q is phenyl. In certain embodiments, Q is naphthyl. In certain embodiments, Q is indenyl. In certain embodiments, Q is indanyl. In certain embodiments, Q is tetralinyl. In certain embodiments, Q is C₃₋₁₀ cycloalkyl. In certain embodiments, Q is 3- to 10-membered heterocyclyl. In certain embodiments, Q is 8- to 11-membered heterobicyclyl. In certain embodiments, Q is substituted with one or more —R¹¹. In certain embodiments, Q is not substituted with —R¹¹.

In certain embodiments, —R^{5′} is selected from the group consisting of —H, C₁₋₆ alkyl, C₂₋₆ alkenyl and C₂₋₆ alkynyl. In certain embodiments, —R^{5′} is selected from the group consisting of —H and C₁₋₆ alkyl. In certain embodiments, —R^{5′} is —H. In certain embodiments, —R^{5′} is C₁₋₆ alkyl.

In certain embodiments, —R^{6′}, —R⁷, —R⁸, —R⁹, —R¹⁰, —R^{10a} are independently selected from the group consisting of —H, C₁₋₁₀ alkyl, C₂₋₁₀ alkenyl, C₂₋₁₀ alkynyl and -Q, wherein -Q is as defined elsewhere herein.

In certain embodiments, —R⁷ and —R⁹ are independently selected from the group consisting of —H and C₁₋₆ alkyl. In certain embodiments, both —R⁷ and —R⁹ are —H. In certain embodiments, —R⁷ is —H. In certain embodiments, —R⁷ is C₁₋₆ alkyl. In certain embodiments, —R⁹ is —H. In certain embodiments, —R⁹ is C₁₋₆ alkyl.

In certain embodiments, —R¹⁰ and —R^{10a} are independently selected from the group consisting of —H and C₁₋₆ alkyl. In certain embodiments, both —R¹⁰ and —R^{10a} are —H. In certain embodiments, —R¹⁰ is —H. In certain embodiments, —R¹⁰ is C₁₋₆ alkyl. In certain embodiments, —R^{10a} is —H. In certain embodiments, —R^{10a} is C₁₋₆ alkyl.

In certain embodiments, -Tr is

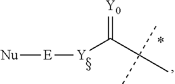

wherein the dashed line marked with an asterisk indicates the attachment to —Y₃—; and
-Nu, -E-, —Y_{§} — and ═Y₀ are as defined elsewhere herein. It is understood that in this instance the release of -D is not triggered by an enzyme, and that DH is released in its unmodified, pharmacologically active form in the absence of an enzyme.

In certain embodiments, -Tr is

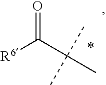

wherein and the dashed line marked with an asterisk indicates the attachment to —Y³— and —R^{6′} is as defined elsewhere herein.

In certain embodiments, —R^{6′} is

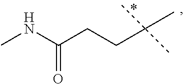

wherein the dashed line marked with the asterisk indicates the attachment to the carbon atom substituted by the oxygen atom.

In certain embodiments —R^{6′} is of formula (a′):

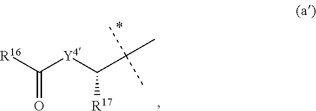

wherein —Y^{4′}— is selected from the group consisting of C₃₋₁₀ cycloalkyl, 3- to 10-membered heterocyclyl and 8- to 11-membered heterobicyclyl, which are optionally substituted with one or more —R¹⁸ which are the same or different;
—R¹⁶ and —R¹⁷ are independently selected from the group consisting of —H, C₁₋₁₀ alkyl, C₂₋₁₀ alkenyl and C₂₋₁₀ alkynyl; wherein C₁₋₁₀ alkyl, C₂₋₁₀ alkenyl and C₂₋₁₀ alkynyl are optionally substituted with one or more —R¹⁸ which are the same or different; and wherein C₁₋₁₀ alkyl, C₂₋₁₀ alkenyl and C₂₋₁₀ alkynyl are optionally interrupted by one or more groups selected from the group consisting of -A′-, —C(O)O—, —O—, —C(O)—, —C(O)N(R¹⁹)—, —S(O)₂N(R¹⁹), —S(O)N(R¹⁹)—, —S(O)₂—, —S(O)—, —N(R¹⁹)S(O)₂N(R^{19a})—, —S—, —N(R¹⁹)—, —OC(OR¹⁹)R^{19a}—, —N(R¹⁹)C(O)N(R^{19a})—, —OC(O)N(R¹⁹)— and —N(R¹⁹)C(NH₂)N(R^{19a})—;
   each A′ is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each A′ is independently optionally substituted with one or more —R¹⁸ which are the same or different;
   wherein —R¹⁸, —R¹⁹ and —R^{19a} are independently selected from the group consisting of —H and C₁₋₆ alkyl; wherein C₁₋₆ alkyl is optionally substituted with one or more halogen, which are the same or different; and
wherein the dashed line marked with an asterisk indicates the attachment to the rest of -Tr.

In certain embodiments, —Y^{4′}— is selected from the group consisting of C₃₋₁₀ cycloalkyl, 3- to 10-membered heterocyclyl and 8- to 11-membered heterobicyclyl. In certain embodiments, —Y^{4′}— is C₃₋₁₀ cycloalkyl. In certain embodiments, —Y^{4′}— is 3- to 10-membered heterocyclyl. In certain embodiments, —Y^{4′}— is 8- to 11-membered heterobicyclyl. In certain embodiments, —Y^{4′}— is substituted with one or more —R¹⁸ which are the same or different. In certain embodiments, —Y^{4′}— is not substituted with —R¹⁸.

In certain embodiments, —R¹⁶ and —R¹⁷ are selected from the group consisting of C₁₋₁₀ alkyl, C₂₋₁₀ alkenyl and C₂₋₁₀ alkynyl. In certain embodiments, —R¹⁶ is C₁₋₁₀ alkyl. In certain embodiments, —R¹⁶ is C₂₋₁₀ alkenyl. In certain embodiments, —R¹⁶ is C₂₋₁₀ alkynyl. In certain embodiments, —R¹⁷ is C₁₋₁₀ alkyl. In certain embodiments, —R¹⁷ is C₂₋₁₀ alkenyl. In certain embodiments, —R¹⁷ is C₂₋₁₀ alkynyl.

In certain embodiments, A′ is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments, A′ is phenyl. In certain embodiments, A′ is naphthyl.

In certain embodiments, A′ is indenyl. In certain embodiments, A′ is indanyl. In certain embodiments, A′ is tetralinyl. In certain embodiments, A′ is C₃₋₁₀ cycloalkyl. In certain embodiments, A′ is 3- to 10-membered heterocyclyl. In certain embodiments, A′ is 8- to 11-membered heterobicyclyl. In certain embodiments, A′ is substituted with one or more —R¹¹, which are the same or different. In certain embodiments, A′ is not substituted with —R¹⁸.

In certain embodiments, —R¹⁸, —R¹⁹ and —R^{19a} are selected from the group consisting of —H and C₁₋₆ alkyl. In certain embodiments, —R¹⁸ is —H. In certain embodiments, —R¹⁸ is C₁₋₆ alkyl. In certain embodiments, —R¹⁹ is —H. In certain embodiments, —R¹⁹ is C₁₋₆ alkyl. In certain embodiments, —R^{19a} is —H. In certain embodiments, —R^{19a} is C₁₋₆ alkyl.

In certain embodiments, —R^{6′} is of formula (b′):

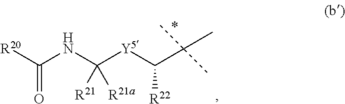

wherein —Y^{5′}— is selected from the group consisting of -Q′-, C₁₋₁₀ alkyl, C₂₋₁₀ alkenyl and C₂₋₁₀ alkynyl; wherein C₁₁io alkyl, C₂₋₁₀ alkenyl and C₂₋₁₀ alkynyl are optionally substituted with one or more —R²³, which are the same or different; and wherein C₁₋₁₀ alkyl, C₂₋₁₀ alkenyl and C₂₋₁₀ alkynyl are optionally interrupted by one or more groups selected from the group consisting of -Q′-, —C(O)O—, —O—, —C(O)—, —C(O)N(R²⁴)—, —S(O)₂N(R²⁴)—, —S(O)N(R²⁴)—, —S(O)₂—, —S(O)—, —N(R²⁴)S(O)₂N(R^{24a})—, —S—, —N(R²⁴)—, —OC(OR²⁴)R^{24a}—, —N(R²⁴)C(O)N(R^{24a})—, —OC(O)N(R²⁴)— and —N(R²⁴)C(NH₂)N(R^{24a})—;
—R²⁰, —R²¹, —R^{21a} and —R²² are independently selected from the group consisting of —H, C₁₋₁₀ alkyl, C₂₋₁₀ alkenyl and C₂₋₁₀ alkynyl; wherein C₁₋₁₀ alkyl, C₂₋₁₀ alkenyl and C₂₋₁₀ alkynyl are optionally substituted with one or more —R²³ which are the same or different; and wherein C₁₋₁₀ alkyl, C₂₋₁₀ alkenyl and C₂₋₁₀ alkynyl are optionally interrupted by one or more groups selected from the group consisting of -Q′-, —C(O)O—, —O—, —C(O)—, —C(O)N(R²⁴)—, —S(O)₂N(R²⁴)—, —S(O)N(R²⁴)—, —S(O)₂—, —S(O)—, —N(R²⁴)S(O)₂N(R^{24a})—, —S—, —N(R²⁴)—, —OC(OR²⁴)R^{24a}—, —N(R²⁴)C(O)N(R^{24a})—, —OC(O)N(R²⁴)— and —N(R²⁴)C(NH₂)N(R^{24a})—;
   each Q′ is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each Q′ is independently optionally substituted with one or more —R²³, which are the same or different;
   wherein —R²³, —R²⁴ and —R^{24a} are independently selected from the group consisting of —H and C₁₋₆ alkyl; wherein C₁₋₆ alkyl is optionally substituted with one or more halogen, which are the same or different;
optionally, the pair —R²¹/—R^{21a} is joined together with the atoms to which is attached to form a C₃₋₁₀ cycloalkyl, 3- to 10-membered heterocyclyl or an 8- to 11-membered heterobicyclyl; and
wherein the dashed line marked with an asterisk indicates the attachment to the rest of -Tr.

In certain embodiments, —Y^{5′}— is selected from the group consisting of -Q′-, C₁₋₁₀ alkyl, C₂₋₁₀ alkenyl and C₂₋₁₀ alkynyl. In certain embodiments, —Y^{5′}— is -Q′-. In certain embodiments, —Y^{5′}— is C₁₋₁₀ alkyl. In certain embodiments, —Y^{5′}— is C₂₋₁₀ alkenyl. In certain embodiments, —Y^{5′}— is C₂₋₁₀ alkynyl.

In certain embodiments, Q′ is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments, Q′ is phenyl. In certain embodiments, Q′ is naphthyl. In certain embodiments, Q′ is indenyl. In certain embodiments, Q′ is indanyl. In certain embodiments, Q′ is C₃₋₁₀ cycloalkyl. In certain embodiments, Q′ is 3- to 10-membered heterocyclyl. In certain embodiments, Q′ is 8- to 11-membered heterobicyclyl. In certain embodiments, Q′ is substituted with one or more —R²³ which are the same or different. In certain embodiments, Q′ is not substituted with —R²³.

In certain embodiments, —R²⁰, —R²¹, —R^{21a} and —R²² are selected from the group consisting of —H, C₁₋₁₀ alkyl, C₂₋₁₀ alkenyl and C₂₋₁₀ alkynyl. In certain embodiments, —R²⁰ is —H. In certain embodiments, —R²⁰ is C₁₋₁₀ alkyl. In certain embodiments, —R²⁰ is C₂₋₁₀ alkenyl. In certain embodiments, —R²⁰ is C₂₋₁₀ alkynyl. In certain embodiments, —R²¹ is —H. In certain embodiments, —R²¹ is C₁₋₁₀ alkyl. In certain embodiments, —R²¹ is C₂₋₁₀ alkenyl. In certain embodiments, —R²¹ is C₂₋₁₀ alkynyl. In certain embodiments, —R^{21a} is —H. In certain embodiments, —R^{21a} is C₁₋₁₀ alkyl. In certain embodiments, —R^{21a} is C₂₋₁₀ alkenyl. In certain embodiments, —R^{21a} is C₂₋₁₀ alkynyl. In certain embodiments, —R²² is —H. In certain embodiments, —R²² is C₁₋₁₀ alkyl. In certain embodiments, —R²² is C₂₋₁₀ alkenyl. In certain embodiments, —R²² is C₂₋₁₀ alkynyl. In certain embodiments, —R²³, —R²⁴ and —R^{24a} are selected from the group consisting of —H and C₁₋₆ alkyl. In certain embodiments, —R²³ is —H. In certain embodiments, —R²³ is C₁₋₆ alkyl. In certain embodiments, —R²⁴ is —H. In certain embodiments, —R²⁴ is C₁₋₆ alkyl. In certain embodiments, —R^{24a} is —H. In certain embodiments, —R^{24a} is C₁₋₆ alkyl.

In certain embodiments, the pair —R²¹/—R^{21a} is joined together with the atoms to which is attached to form a C₃₋₁₀ cycloalkyl.

In certain embodiments, —R^{6′} is of formula (c):

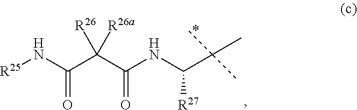

wherein
—R²⁵, —R²⁶, —R^{26a} and —R²⁷ are independently selected from the group consisting of —H, C₁₋₁₀ alkyl, C₂₋₁₀ alkenyl and C₂₋₁₀ alkynyl; wherein C₁₋₁₀ alkyl, C₂₋₁₀ alkenyl and C₂₋₁₀ alkynyl are optionally substituted with one or more —R²⁸ which are the same or different; and wherein C₁₋₁₀ alkyl, C₂₋₁₀ alkenyl and C₂₋₁₀ alkynyl are optionally interrupted by one or more groups selected from the group consisting of -Q*-, —C(O)O—, —O—, —C(O)—, —C(O)N(R²⁹)—, —S(O)₂N(R²⁹)—, —S(O)N(R²⁹)—, —S(O)₂—, —S(O)—, —N(R²⁹)S(O)₂N(R^{29a})—, —S—, —N(R²⁹)—, —OC(OR²⁹)R^{29a}—, —N(R²⁹)C(O)N(R^{29a})—, —OC(O)N(R²⁹)— and —N(R²⁹)C(NH₂)N(R^{29a})—;
   each Q* is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each Q* is independently optionally substituted with one or more —R²⁸, which are the same or different;
   wherein —R²⁸, —R²⁹ and —R^{29a} are independently selected from the group consisting of —H and C₁₋₆ alkyl; wherein C₁₋₆ alkyl is optionally substituted with one or more halogen, which are the same or different;
optionally, the pair —R²⁶/—R^{26a} is joined together with the atoms to which is attached to form a C₃₋₁₀ cycloalkyl, 3- to 10-membered heterocyclyl or an 8- to 11-membered heterobicyclyl; and
wherein the dashed line marked with an asterisk indicates the attachment to the rest of -Tr.

In certain embodiments, —R²⁵, —R²⁶, —R^{26a} and —R²⁷ are selected from the group consisting of —H, C₁₋₁₀ alkyl, C₂₋₁₀ alkenyl and C₂₋₁₀ alkynyl. In certain embodiments, —R²⁵ is —H. In certain embodiments, —R²⁵ is C₁₋₁₀ alkyl. In certain embodiments, —R²⁵ is C₂₋₁₀ alkenyl. In certain embodiments, —R²⁵ is C₂₋₁₀ alkynyl. In certain embodiments, —R²⁶ is —H. In certain embodiments, —R²⁶ is C₁₋₁₀ alkyl. In certain embodiments, —R²⁶ is C₂₋₁₀ alkenyl. In certain embodiments, —R²⁶ is C₂₋₁₀ alkynyl. In certain embodiments, —R^{26a} is —H. In certain embodiments, —R^{26a} is C₁₋₁₀ alkyl. In certain embodiments, —R^{26a} is C₂₋₁₀ alkenyl. In certain embodiments, —R^{26a} is C₂₋₁₀ alkynyl. In certain embodiments, —R²⁷ is —H. In certain embodiments, —R²⁷ is C₁₋₁₀ alkyl. In certain embodiments, —R²⁷ is C₂₋₁₀ alkenyl. In certain embodiments, —R²⁷ is C₂₋₁₀ alkynyl.

In certain embodiments, Q* is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments, Q* is phenyl. In certain embodiments, Q* is naphthyl. In certain embodiments, Q* is indenyl. In certain embodiments, Q* is indanyl. In certain embodiments, Q* is tetralinyl. In certain embodiments, Q* is C₃₋₁₀ cycloalkyl. In certain embodiments, Q* is 3- to 10-membered heterocyclyl. In certain embodiments, Q* is 8-to 11-membered heterobicyclyl. In certain embodiments, Q* is substituted with one or more —R²⁸, which are the same or different. In certain embodiments, Q* is not substituted with —R²⁸.

In certain embodiments, —R²⁸, —R²⁹ and —R^{29a} are selected from the group consisting of —H and C₁₋₆ alkyl. In certain embodiments, —R²⁸ is —H. In certain embodiments, —R²⁸ is C₁₋₆ alkyl. In certain embodiments, —R²⁹ is —H. In certain embodiments, —R²⁹ is C₁₋₆ alkyl. In certain embodiments, —R^{29a} is —H. In certain embodiments, —R^{29a} is C₁₋₆ alkyl.

In certain embodiments, the pair —R²⁶/—R^{26a} is joined together with the atoms to which is attached to form a C₃₋₁₀ cycloalkyl. In certain embodiments, the pair —R²⁶/—R^{26a} is joined together with the atoms to which is attached to form a cyclobutyl.

In certain embodiments, -Tr is

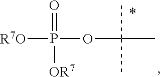

wherein the dashed line marked with an asterisk indicates the attachment to —Y³— and —R⁷ is defined as elsewhere herein. It is understood that in this instance the release of -D may be triggered by an enzyme, such as phosphatase.

In certain embodiments, -Tr is

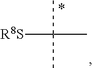

wherein the dashed line marked with an asterisk indicates the attachment to —Y³— which is —S— and —R⁸ is as defined elsewhere herein.

In certain embodiments, -Tr is

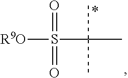

wherein the dashed line marked with an asterisk indicates the attachment to —Y³— and —R⁹ is as defined elsewhere herein. It is understood that in this instance the release of -D may be triggered by an enzyme, such as sulfatase.

In certain embodiments, -Tr is

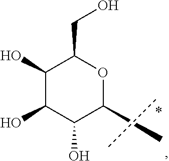

wherein the dashed line marked with an asterisk indicates the attachment to —Y³—. It is understood that in this instance the release of -D may be triggered by an enzyme, such as α-galactosidase.

In certain embodiments, -Tr is

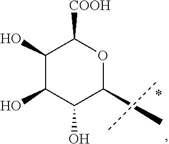

wherein the dashed line marked with an asterisk indicates the attachment to —Y³—. It is understood that in this instance the release of -D may be triggered by an enzyme, such as β-glucuronidase.

In certain embodiments, -Tr is

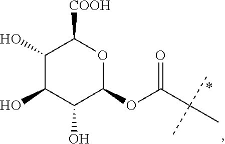

wherein the dashed line marked with an asterisk indicates the attachment to —Y³—. It is understood that in this instance the release of the -D may be triggered by an enzyme, such as β-glucuronidase.

In certain embodiments, -Tr is a peptidyl moiety. It is understood that if -Tr is a peptidyl moiety, then the release of the -D may be triggered by an enzyme, such as protease. In certain embodiments, the protease is selected from the group consisting of cathepsin B and cathepsin K. In certain embodiments, the protease is cathepsin B. In certain embodiments, the protease is cathepsin K.

In certain embodiments, -Tr is a peptidyl moiety, such as a dipeptidyl, tripeptidyl, tetrapeptidyl, pentapeptidyl or hexapeptidyl moiety. In certain embodiments, -Tr is a dipeptidyl moiety. In certain embodiments, -Tr is a tripeptidyl moiety. In certain embodiments, -Tr is a tetrapeptidyl moiety. In certain embodiments, -Tr is a pentapeptidyl moiety. In certain embodiments, -Tr is a hexapeptidyl moiety.

In certain embodiments, -Tr is a peptidyl moiety selected from the group consisting of:

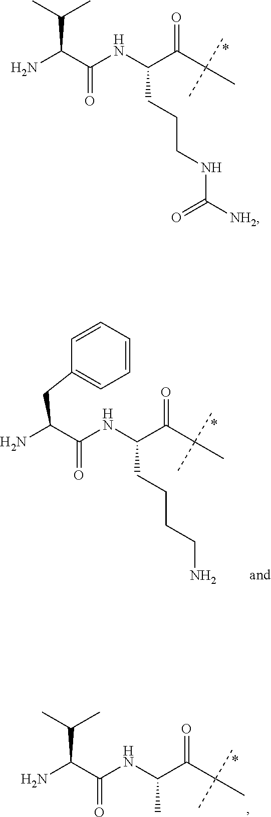

wherein the dashed line marked with an asterisk indicates the attachment to —Y³—.

In certain embodiments, -Tr is

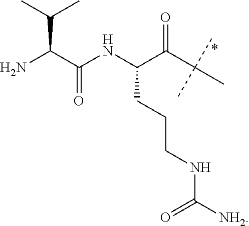

In certain embodiments, -Tr is

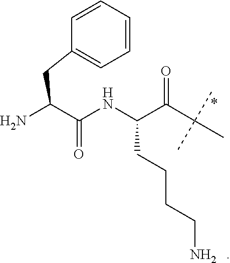

In certain embodiments, -Tr is

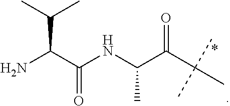

In certain embodiments, —Y₃— is —N(R⁶)— and -Tr has the structure of -L¹- as disclosed in WO 2011/012722 A1, which is herewith incorporated by reference. Accordingly, in certain embodiments, -Tr is of formula (a012):

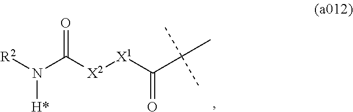

wherein the dashed line indicates the attachment —Y₃—;
X¹ is C(R¹R^{1a}) or a cyclic fragment selected from C₃₋₇ cycloalkyl, 4 to 7 membered heterocyclyl, phenyl, naphthyl, indenyl, indanyl, tetralinyl, or 9 to 11 membered heterobicyclyl,
wherein in case X¹ is a cyclic fragment, said cyclic fragment is incorporated into L¹ via two adjacent ring atoms and the ring atom of X¹, which is adjacent to the carbon atom of the amide bond, is also a carbon atom;
X² is a chemical bond or selected from C(R³R^{3a}), N(R³), O, C(R³R^{3a})—C(R⁴R^{4a}), C(R³R^{3a})—N(R⁴), N(R³)—C(R⁴R^{4a}), C(R³R^{3a})—O, or O—C(R³R^{3a}), wherein in case X¹ is a cyclic fragment, X² is a chemical bond, C(R³R^{3a}), N(R³) or O; optionally, in case X¹ is a cyclic fragment and X² is C(R³R^{3a}), the order of the X¹ fragment and the X² fragment within L¹ may be changed and the cyclic fragment is incorporated into L¹ via two adjacent ring atoms;
R¹, R³ and R⁴ are independently selected from the group consisting of H, C₁₋₄ alkyl and —N(R⁵R^{5a});
R^{1a}, R², R^{3a}, R^{4a} and R^{5a} are independently selected from the group consisting of H, and C₁₋₄ alkyl;
R⁵ is C(O)R⁶;
R⁶ is C₁₋₄ alkyl; and optionally, one of the pairs R^{1a}/R^{4a}, R^{3a}/R^{4a} or R^{1a}/R^{3a} form a chemical bond.

In certain embodiments, -Tr and —Y₃— form together a functional group selected from the group consisting of

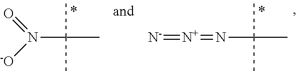

wherein the dashed line marked with the asterisk indicates the attachment to —Ar—.

In certain embodiments, -Tr is

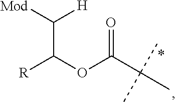

wherein -Mod and —R are as defined elsewhere herein.

Suitably, -Mod is MeSO₂—.

In certain embodiments, -Tr is selected from the group consisting of

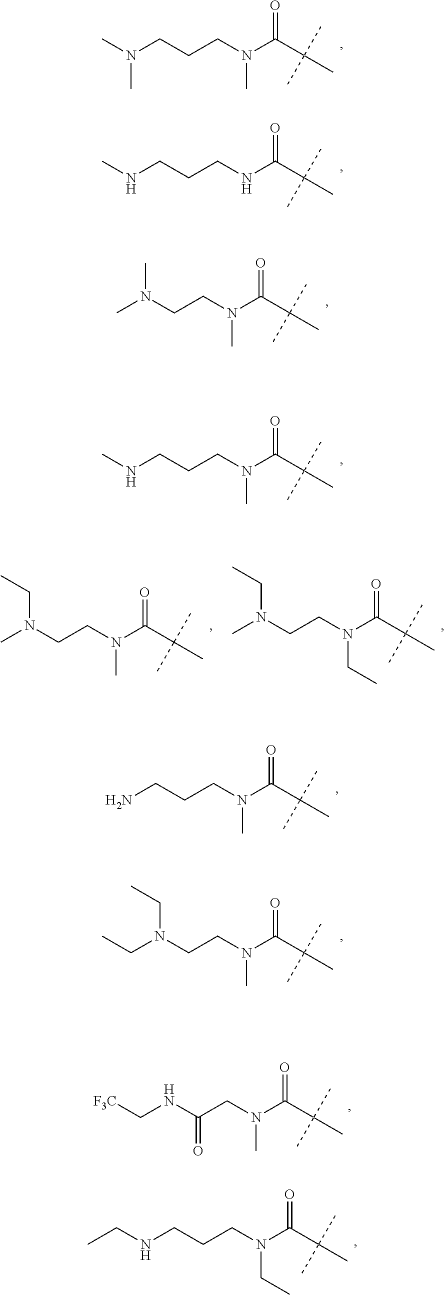

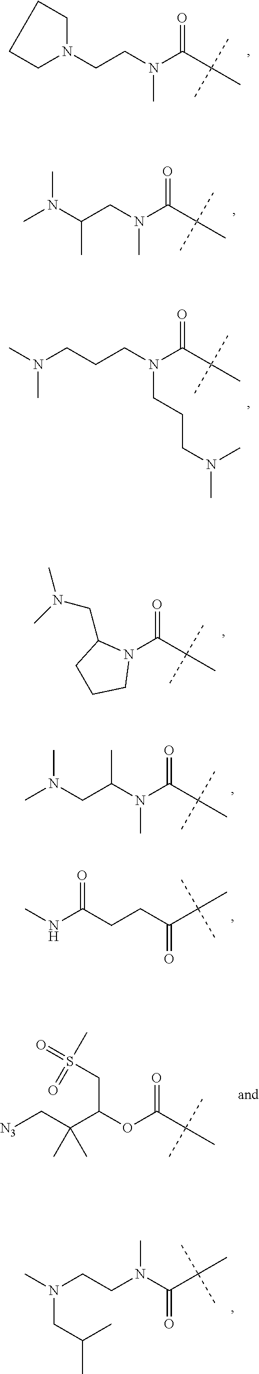

wherein the dashed line indicates the attachment to —Y³— as defined elsewhere herein.

The drug conjugate or pharmaceutically acceptable salt thereof according to the present invention may be of formula (IIa):

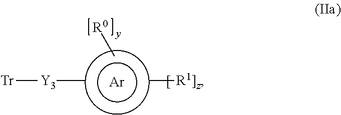

wherein
each —R¹ is independently selected from the group consisting of

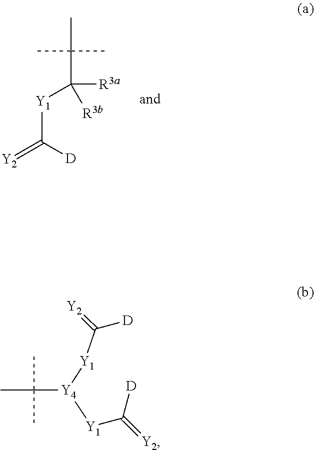

wherein the dashed line indicates the attachment to —Ar—;
z is selected from the group consisting of 2 and 3;
y is selected from the group consisting of 0, 1, 2, 3, 4 and 5;
each -D is a drug moiety comprising an albumin-binding moiety as described elsewhere herein, wherein the total number of -Ds within the drug conjugate is ranging from 2 to 6;
-Tr is a cleavable triggering moiety as described elsewhere herein;
each —Y₁— is independently selected from the group consisting of —O— and —S—; —Y₃— is independently selected from the group consisting of —O—, —S— and —N(R⁶)—;
each ═Y₂ is independently selected from the group consisting of ═O and ═S;
—Ar— is a ring selected from the group consisting of monocyclic or bicyclic aryl and heteroaryl, provided that —Ar— is connected to —Y₃— and —R¹ via carbon atoms; wherein said monocyclic or bicyclic aryl and heteroaryl are optionally substituted with —R⁰, which are the same or different;
—Y₄— is

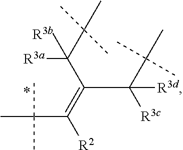

wherein the dashed line marked with the asterisk indicates the attachment to —Ar— and the unmarked dashed lines indicate the attachment to —Y₁—;
each —R⁰ is independently selected from the group consisting of —C(O)OH, -halogen, —NO₂, —CN, C₁₋₃₀ alkyl, C₂₋₃₀ alkenyl and C₂₋₃₀ alkynyl; wherein C₁₋₃₀ alkyl, C₂₋₃₀ alkenyl and C₂₋₃₀ alkynyl are optionally substituted with one or more —R⁴, which are the same or different; and wherein C₁₋₃₀ alkyl, C₂₋₃₀ alkenyl and C₂₋₃₀ alkynyl are optionally interrupted by one or more groups selected from the group consisting of -T-, —C(O)O—, —O—, —C(O)—, —C(O)N(R⁵)—, —S(O)₂N(R⁵)—, —S(O)N(R⁵)—, —S(O)₂—, —S(O)—, —N(R⁵)S(O)₂N(R^{5a})—, —S—, —N(R⁵)—, —OC(OR⁵)(R^{5a})—, —N(R⁵)C(O)N(R^{5a})— and —OC(O)N(R⁵)—;
each —R² is independently selected from the group consisting of —H, —C(O)OH, -halogen, —NO₂, —CN, C₁₋₃₀ alkyl, C₂₋₃₀ alkenyl and C₂₋₃₀ alkynyl; wherein C₁₋₃₀ alkyl, C₂₋₃₀ alkenyl and C₂₋₃₀ alkynyl are optionally substituted with one or more —R⁴, which are the same or different; and wherein C₁₋₃₀ alkyl, C₂₋₃₀ alkenyl and C₂₋₃₀ alkynyl are optionally interrupted by one or more groups selected from the group consisting of -T-, —C(O)O—, —O—, —C(O)—, —C(O)N(R⁵)—, —S(O)₂N(R⁵)—, —S(O)N(R⁵)—, —S(O)₂—, —S(O)—, —N(R⁵)S(O)₂N(R^{5a})—, —S—, —N(R⁵)—, —OC(OR⁵)(R^{5a})—, —N(R⁵)C(O)N(R^{5a})— and —OC(O)N(R⁵)—;
each —R^{3a}, —R^{3b}, —R^{3c}, —R^{3d} are independently selected from the group consisting of —H, —C(O)OH, —F, —NO₂, —CN, C₁₋₃₀ alkyl, C₂₋₃₀ alkenyl and C₂₋₃₀ alkynyl; wherein C₁₋₃₀ alkyl, C₂₋₃₀ alkenyl and C₂₋₃₀ alkynyl are optionally substituted with one or more —R⁴, which are the same or different; and wherein C₁₋₃₀ alkyl, C₂₋₃₀ alkenyl and C₂₋₃₀ alkynyl are optionally interrupted by one or more groups selected from the group consisting of -T-, —C(O)O—, —O—, —C(O)—, —C(O)N(R⁵)—, —S(O)₂N(R⁵)—, —S(O)N(R⁵)—, —S(O)₂—, —S(O)—, —N(R⁵)S(O)₂N(R^{5a})—, —S—, —N(R⁵)—, —OC(OR⁵)(R^{5a})—, —N(R⁵)C(O)N(R^{5a})— and —OC(O)N(R⁵)—;
each -T- is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each -T- is independently optionally substituted with one or more —R⁴, which are the same or different; wherein —R⁴ is selected from the group consisting of —NO₂, —OCH₃, —CN, —N(R⁵)(R^{5a}), —OH, —C(O)OH and C₁₋₆ alkyl; wherein C₁₋₆ alkyl is optionally substituted with one or more halogen, which are the same or different;
—R⁵ and —R^{5a} are independently selected from the group consisting of —H and C₁₋₆ alkyl; wherein C₁₋₆ alkyl is optionally substituted with one or more halogen, which are the same or different; and
each —R⁶ is independently selected from the group consisting of —H, C₁₋₆ alkyl and —C(R^{3a})(R^{3b})—BB, wherein —BB is defined as elsewhere herein.

In certain embodiments, all —R¹ of formula (IIa) are

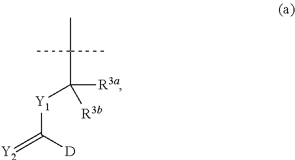

wherein —R^{3a}, —R^{3b}, —Y₁—, =Y₂ and -D are as described elsewhere herein.

In certain embodiments, all —R¹ of formula (IIa) are

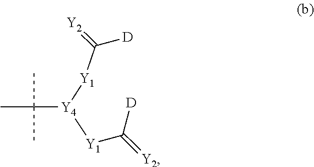

wherein —Y₄—, —Y₁—, =Y₂ and -D are as described elsewhere herein.

In certain embodiments, z of formula (IIa) is 2. In certain embodiments, z of formula (IIa) is 3.

In certain embodiments, z of formula (IIa) is 2 and both —R¹ are of formula (a). In certain embodiments, z of formula (IIa) is 2 and both —R¹ are of formula (b). In certain embodiments, z is of formula (IIa) is 2 and one —R¹ is of formula (a) and one —R¹ is of formula (b).

In certain embodiments, z of formula (IIa) is 3 and all —R¹ are of formula (a). In certain embodiments, z of formula (IIa) is 3 and all —R¹ are of formula (b). In certain embodiments, z of formula (IIa) is 3 and one —R¹ is of formula (a) and two —R¹ are of formula (b). In certain embodiments, z of formula (IIa) is 3 and two —R¹ are of formula (a) and one —R¹ is of formula (b).

In certain embodiments, y of formula (IIa) is 0. In certain embodiments, y of formula (IIa) is 1. In certain embodiments, y of formula (IIa) is 2. In certain embodiments, y of formula (IIa) is 3. In certain embodiments, y of formula (IIa) is 4. In certain embodiments, y of formula (IIa) is 5.

In certain embodiments, z of formula (IIa) is 2, both —R¹ of formula (IIa) are of formula (a) and y of formula (IIa) is 0. In certain embodiments, z of formula (IIa) is 3, all —R¹ of formula (IIa) are of formula (a) and y of formula (IIa) is 0. In certain embodiments, z of formula (IIa) is 3, two —R¹ are of formula (a), one —R¹ of formula (IIa) is of formula (b) and y of formula (IIa) is 0.

In certain embodiments, —Y₁— of formula (IIa) is —O—. In certain embodiments, —Y₁— of formula (IIa) is —S—.

In certain embodiments, ═Y₂ of formula (IIa) is ═O. In certain embodiments, ═Y₂ of formula (IIa) is ═S.

In certain embodiments, —Y₃— of formula (IIa) is —O—. In certain embodiments, —Y₃— of formula (IIa) is —S—. In certain embodiments, —Y₃— of formula (IIa) is —N(R⁶)—, wherein —R⁶ is as described elsewhere herein.

In certain embodiments, -Tr of formula (IIa) is

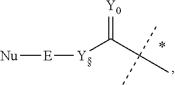

wherein -Nu, -E, —Y_{§} — an ═Y₀ are as described elsewhere herein and the dashed line marked with an asterisk indicates the attachment to —Y₃—.

The drug conjugate or pharmaceutically acceptable salt thereof according to the present invention may be of formula (IIa1):

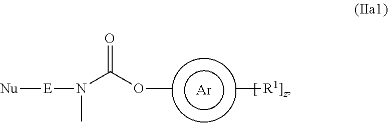

wherein
each —R¹ is independently selected from the group consisting of

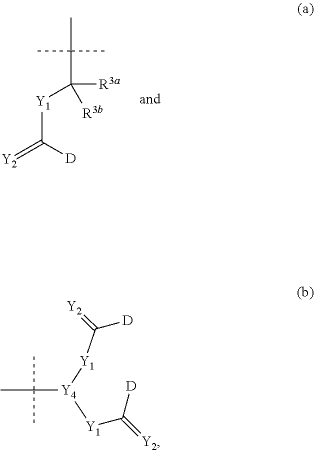

wherein the dashed line indicates the attachment to —Ar—;
z is selected from the group consisting of 2 and 3;
each -D is a drug moiety comprising an albumin-binding moiety as described elsewhere herein, wherein the total number of -Ds within the drug conjugate is ranging from 2 to 6;
each —Y₁— is —O—;
each ═Y₂ is ═O; and
-Nu, -E, —Ar—, —Y₄—, —R^{3a} and —R^{3b} are as described elsewhere herein.

In certain embodiments, all —R¹ of formula (IIa1) are

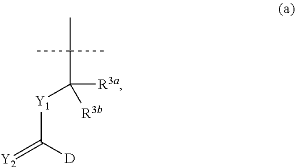

wherein —R^{3a}, —R^{3b}, —Y₁—, ═Y₂ and -D are as described elsewhere herein.

In certain embodiments, all —R¹ of formula (IIa1) are

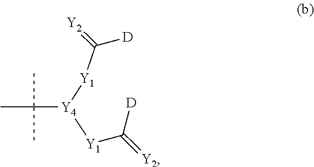

wherein —Y₄—, —Y₁—, ═Y₂ and -D are as described elsewhere herein.

The drug conjugate or pharmaceutically acceptable salt thereof according to the present invention may be of formula (IIa2):

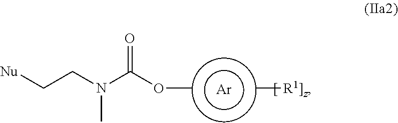

wherein
each —R¹ is independently selected from the group consisting of

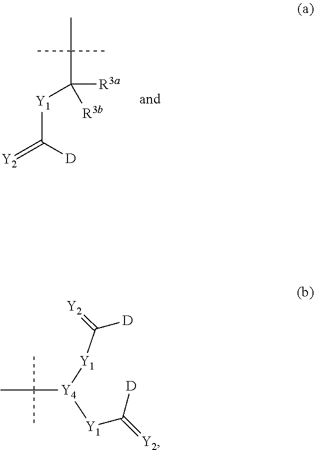

wherein the dashed line indicates the attachment to —Ar—;
z is selected from the group consisting of 2 and 3;
each -D is a drug moiety comprising an albumin-binding moiety as described elsewhere herein, wherein the total number of -Ds within the drug conjugate is ranging from 2 to 6;
-Nu is a secondary or tertiary amine functional group;
each —Y₁— is —O—;
each ═Y₂ is ═O; and
—Ar—, —Y₄—, —R^{3a} and —R^{3b} are as described elsewhere herein.

In certain embodiments, all —R¹ of formula (IIa2) are

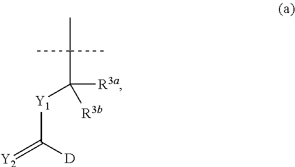

wherein —R^{3a}, —R^{3b}, —Y₁—, ═Y₂ and -D are as described elsewhere herein.

In certain embodiments, all —R¹ of formula (IIa2) are

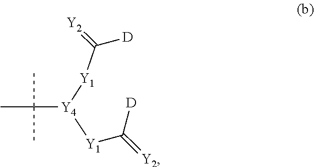

wherein —Y₄—, —Y₁—, ═Y₂ and -D are as described elsewhere herein.

In certain embodiments, the drug conjugate or pharmaceutically acceptable salt thereof of formula (IIa) is selected from the group consisting of

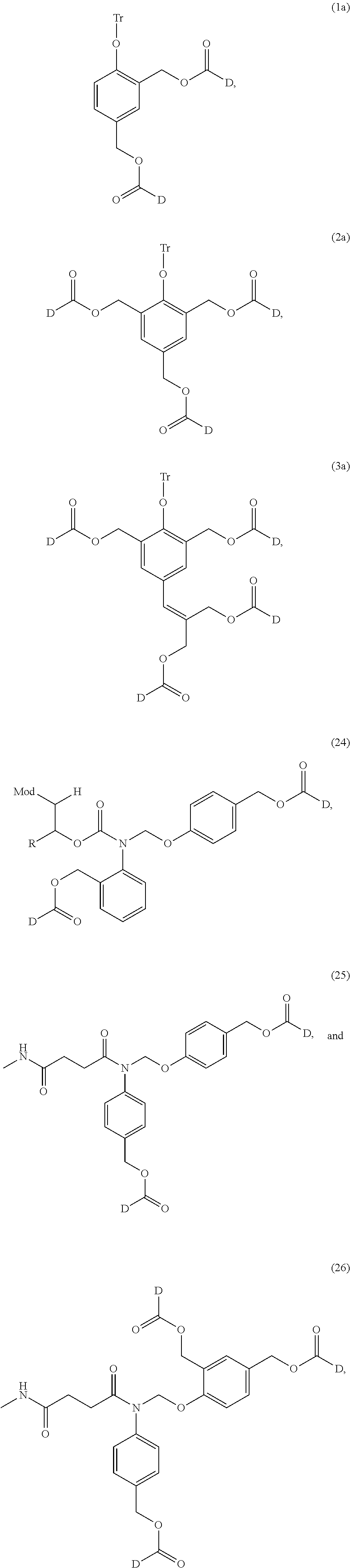

wherein each -D is a drug moiety comprising an albumin-binding moiety as described elsewhere herein;
—R and -Mod are as described elsewhere herein; and
-Tr is selected from the group consisting of

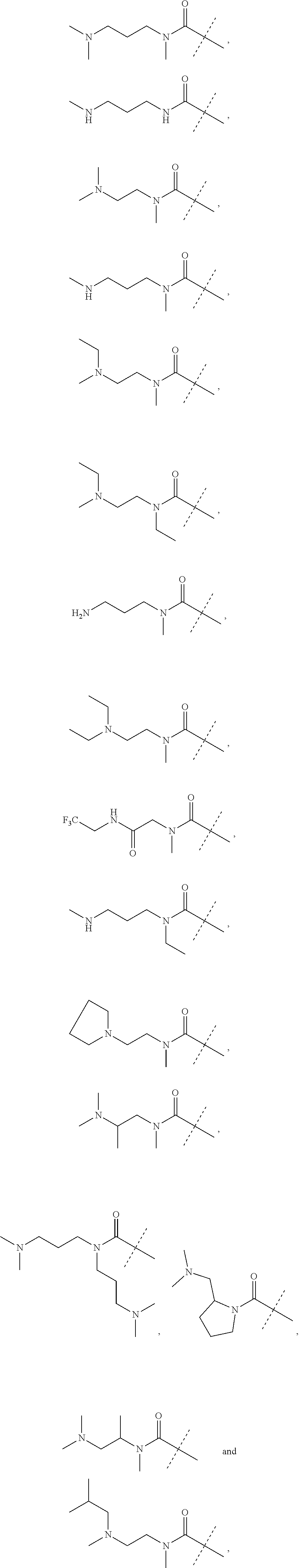

wherein the dashed line indicates the attachment to the oxygen atom.

The drug conjugate or pharmaceutically acceptable salt thereof according to the present invention may be of formula (1Ib):

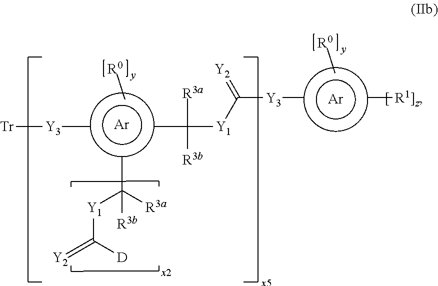

wherein
each —R¹ is independently selected from the group consisting of

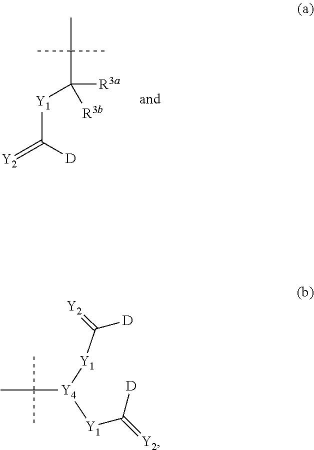

wherein the dashed line indicates the attachment to —Ar—;
x2 is selected from the group consisting of 0, 1 and 2;
z is selected from the group consisting of 1, 2 and 3;
each y is independently selected from the group consisting of 0, 1, 2, 3, 4 and 5;
x5 is selected from the group consisting of 1, 2, 3, 4 and 5;
each -D is a drug moiety comprising an albumin-binding moiety, wherein the total number of -Ds within the drug conjugate is ranging from 2 to 8;
-Tr is a cleavable triggering moiety as described elsewhere herein;
each —Y₁— is independently selected from the group consisting of —O— and —S—;
each ═Y₂ is independently selected from the group consisting of ═O and ═S;
each —Y₃— is independently selected from the group consisting of —O—, —S— and —N(R⁶)—;
each —Ar— is independently a ring selected from the group consisting of monocyclic or bicyclic aryl and heteroaryl, provided that —Ar— is connected to —Y₃— and —C(R^{3a})(R^{3b})—, via carbon atoms; wherein said monocyclic or bicyclic aryl and heteroaryl are optionally substituted with —R⁰, which are the same or different;
—Y₄— is

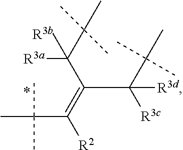

wherein the dashed line marked with the asterisk indicates the attachment to —Ar— and the unmarked dashed lines indicate the attachment to —Y₁—;
each —R⁰ is independently selected from the group consisting of —C(O)OH, -halogen, —NO₂, —CN, C₁₋₃₀ alkyl, C₂₋₃₀ alkenyl and C₂₋₃₀ alkynyl; wherein C₁₋₃₀ alkyl, C₂₋₃₀ alkenyl and C₂₋₃₀ alkynyl are optionally substituted with one or more —R⁴, which are the same or different; and wherein C₁₋₃₀ alkyl, C₂₋₃₀ alkenyl and C₂₋₃₀ alkynyl are optionally interrupted by one or more groups selected from the group consisting of -T-, —C(O)O—, —O—, —C(O)—, —C(O)N(R⁵)—, —S(O)₂N(R⁵)—, —S(O)N(R⁵)—, —S(O)₂—, —S(O)—, —N(R⁵)S(O)₂N(R^{5a})—, —S—, —N(R⁵)—, —OC(OR⁵)(R^{5a})—, —N(R⁵)C(O)N(R^{5a})— and —OC(O)N(R⁵)—;
each —R² is independently selected from the group consisting of —H, —C(O)OH, -halogen, —NO₂, —CN, C₁₋₃₀ alkyl, C₂₋₃₀ alkenyl and C₂₋₃₀ alkynyl; wherein C₁₋₃₀ alkyl, C₂₋₃₀ alkenyl and C₂₋₃₀ alkynyl are optionally substituted with one or more —R⁴, which are the same or different; and wherein C₁₋₃₀ alkyl, C₂₋₃₀ alkenyl and C₂₋₃₀ alkynyl are optionally interrupted by one or more groups selected from the group consisting of -T-, —C(O)O—, —O—, —C(O)—, —C(O)N(R⁵)—, —S(O)₂N(R⁵)—, —S(O)N(R⁵)—, —S(O)₂—, —S(O)—, —N(R⁵)S(O)₂N(R^{5a})—, —S—, —N(R⁵)—, —OC(OR⁵)(R^{5a})—, —N(R⁵)C(O)N(R^{5a})— and —OC(O)N(R⁵)—;
each —R^{3a}, R^{3b}, —R^{3c}, —R^{3d} are independently selected from the group consisting of —H, —C(O)OH, —F, —NO₂, —CN, C₁₋₃₀ alkyl, C₂₋₃₀ alkenyl and C₂₋₃₀ alkynyl; wherein C₁₋₃₀ alkyl, C₂₋₃₀ alkenyl and C₂₋₃₀ alkynyl are optionally substituted with one or more —R⁴, which are the same or different; and wherein C₁₋₃₀ alkyl, C₂₋₃₀ alkenyl and C₂₋₃₀ alkynyl are optionally interrupted by one or more groups selected from the group consisting of -T-, —C(O)O—, —O—, —C(O)—, —C(O)N(R⁵)—, —S(O)₂N(R⁵)—, —S(O)N(R⁵)—, —S(O)₂—, —S(O)—, —N(R⁵)S(O)₂N(R^{5a})—, —S—, —N(R⁵)—, —OC(OR⁵)(R^{5a})—, —N(R⁵)C(O)N(R^{5a})— and —OC(O)N(R⁵)—;
each -T- is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each -T- is independently optionally substituted with one or more —R⁴, which are the same or different; wherein —R⁴ is selected from the group consisting of —NO₂, —OCH₃, —CN, —N(R⁵)(R^{5a}), —OH, —C(O)OH and C₁₋₆ alkyl; wherein C₁₋₆ alkyl is optionally substituted with one or more halogen, which are the same or different;
—R⁵ and —R^{5a} are independently selected from the group consisting of —H and C₁₋₆ alkyl; wherein C₁₋₆ alkyl is optionally substituted with one or more halogen, which are the same or different; and
each —R⁶ is independently selected from the group consisting of —H, C₁₋₆ alkyl and —C(R^{3a})(R^{3b})—BB.

In certain embodiments, all —R¹ of formula (IIb) are

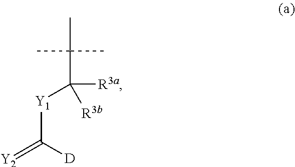

wherein —R^{3a}, —R^{3b}, —Y₁—, ═Y₂ and -D are as described elsewhere herein.

In certain embodiments, all —R¹ of formula (IIb) are

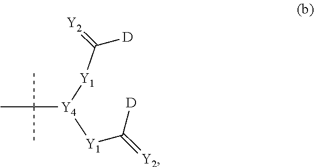

wherein —Y₄—, —Y₁—, ═Y₂ and -D are as described elsewhere herein.

In certain embodiments, x2 of formula (IIb) is selected from the group consisting of 1 and 2. In certain embodiments, x2 of formula (IIb) is 0. In certain embodiments, x2 of formula (IIb) is 1. In certain embodiments, x2 of formula (IIb) is 2. In certain embodiments, x2 of formula (IIb) is 3.

In certain embodiments, z of formula (IIb) is 1. In certain embodiments, z of formula (IIb) is 2. In certain embodiments, z of formula (IIb) is 3.

In certain embodiments, y of formula (IIb) is 0. In certain embodiments, y of formula (IIb) is 1. In certain embodiments, y of formula (IIb) is 2. In certain embodiments, y of formula (IIb) is 3. In certain embodiments, y of formula (IIb) is 4. In certain embodiments, y of formula (IIb) is 5.

In certain embodiments, x5 of formula (IIb) is 1. In certain embodiments, x5 of formula (IIb) is 2. In certain embodiments, x5 of formula (IIb) is 3. In certain embodiments, x5 of formula (IIb) is 4. In certain embodiments, x5 of formula (IIb) is 5.

In certain embodiments, —Y₁— of formula (IIb) is —O—. In certain embodiments, —Y₁— of formula (1Ib) is —S—.

In certain embodiments, ═Y₂ of formula (IIb) is ═O. In certain embodiments, ═Y₂ of formula (1Ib) is ═S.

In certain embodiments, —Y₃— of formula (IIb) is —O—. In certain embodiments, —Y₃— of formula (IIb) is —S—. In certain embodiments, —Y₃— of formula (IIb) is —N(R⁶)—, wherein —R⁶ is as described elsewhere herein.

In certain embodiments, -Tr of formula (IIb) is

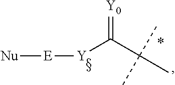

wherein the dashed line marked with an asterisk indicates the attachment to —Y₃—; and
-Nu, -E-, —Y_{§} — and ═Y₀ are as described elsewhere herein.

The drug conjugate or pharmaceutically acceptable salt thereof according to the present invention may be of formula (IIb1):

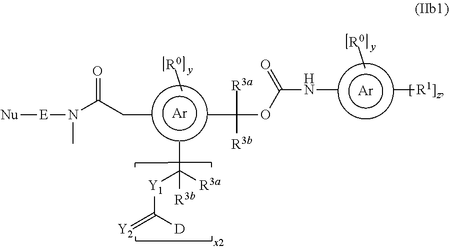

wherein
each —R¹ is independently selected from the group consisting of

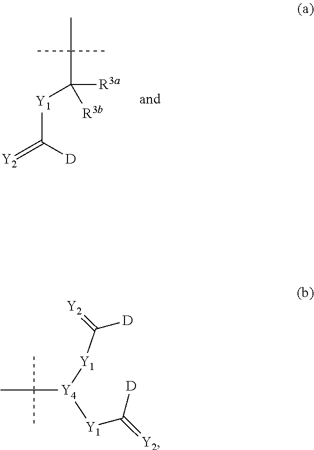

wherein the dashed line indicates the attachment to —Ar—;
x2 is selected from the group consisting of 1 and 2;
z is selected from the group consisting of 1, 2 and 3;
each y is independently selected from the group consisting of 0, 1, 2, 3 and 4;
each -D is a drug moiety comprising an albumin-binding moiety as described elsewhere herein, wherein the total number of -Ds within the drug conjugate is ranging from 2 to 6;
each —Y₁— is —O—;
each ═Y₂ is ═O; and
-Nu, -E-, —Ar—, —Y₄—, —R⁰, —R^{3a} and —R^{3b} are as described elesewhere herein.

In certain embodiments, all —R¹ of formula (IIb1) are

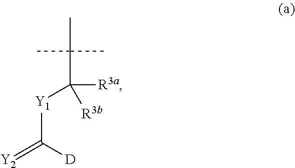

wherein —R^{3a}, —R^{3b}, —Y₁—, ═Y₂ and -D are as described elsewhere herein.

In certain embodiments, all —R¹ of formula (IIb1) are

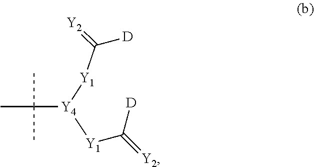

wherein —Y₄—, —Y₁—, ═Y₂ and -D are as described elsewhere herein.

In certain embodiments, two —R¹ of formula (IIb1) are

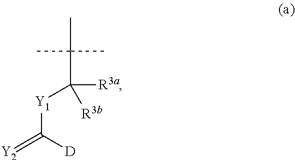

and one is

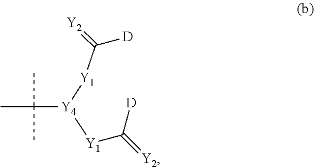

wherein —R^{3a}, R^{3b}, —Y₄—, —Y₁—, —Y₂ and -D are as described elsewhere herein.

The drug conjugate or pharmaceutically acceptable salt thereof according to the present invention may be of formula (IIb2):

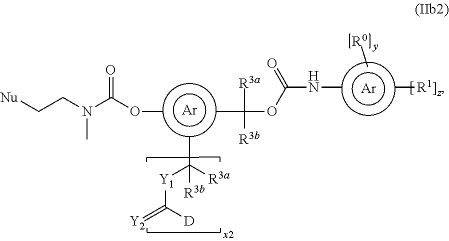

wherein
each —R¹ is independently selected from the group consisting of

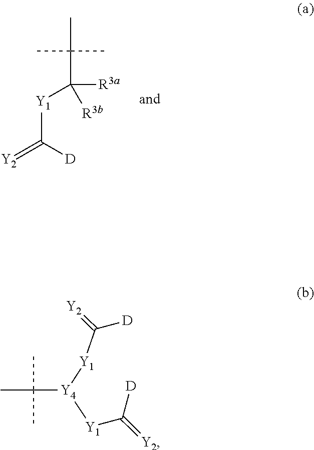

wherein the dashed line indicates the attachment to —Ar—;
x2 is selected from the group consisting of 1 and 2;
z is selected from the group consisting of 1, 2 and 3;
y is selected from the group consisting of 0, 1 and 2;
each -D is a drug moiety comprising an albumin-binding moiety as described elsewhere herein, wherein the total number of -Ds within the drug conjugate is ranging from 2 to 6;
-Nu is a secondary or tertiary amine functional group;
each —Y₁— is —O—;
each ═Y₂ is ═O;
—Ar—, —Y₄—, —R⁰, —R^{3a} and —R^{3b} are as described elsewhere herein.

In certain embodiments, all —R¹ of formula (IIb2) are

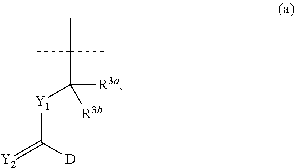

wherein —R^{3a}, —R^{3b}, —Y₁—, ═Y₂ and -D are as described elsewhere herein.

In certain embodiments, all —R¹ of formula (IIb2) are

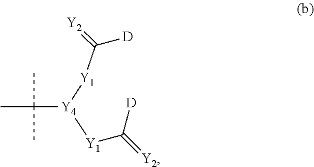

wherein —Y₄, —Y₁, ═Y₂ and -D are as described elsewhere herein.

In certain embodiments, the total number of -Ds within the drug conjugate of formula (IIb), (IIb1) or (IIb2) is 2. In certain embodiments, the total number of -Ds within the drug conjugate of formula (IIb), (IIb1) or (IIb2) is 3. In certain embodiments, the total number of -Ds within the drug conjugate of formula (IIb), (IIb1) or (IIb2) is 4. In certain embodiments, the total number of -Ds within the drug conjugate of formula (IIb), (IIb1) or (IIb2) is 5. In certain embodiments, the total number of -Ds within the drug conjugate of formula (IIb), (IIb1) or (IIb2) is 6. In certain embodiments, the total number of -Ds within the drug conjugate of formula (IIb), (IIb1) or (IIb2) is 7. In certain embodiments, the total number of -Ds within the drug conjugate of formula (IIb), (IIb1) or (IIb2) is 8.

In certain embodiments, the drug conjugate or pharmaceutically acceptable salt thereof of formula (IIb) is selected from the group consisting of:

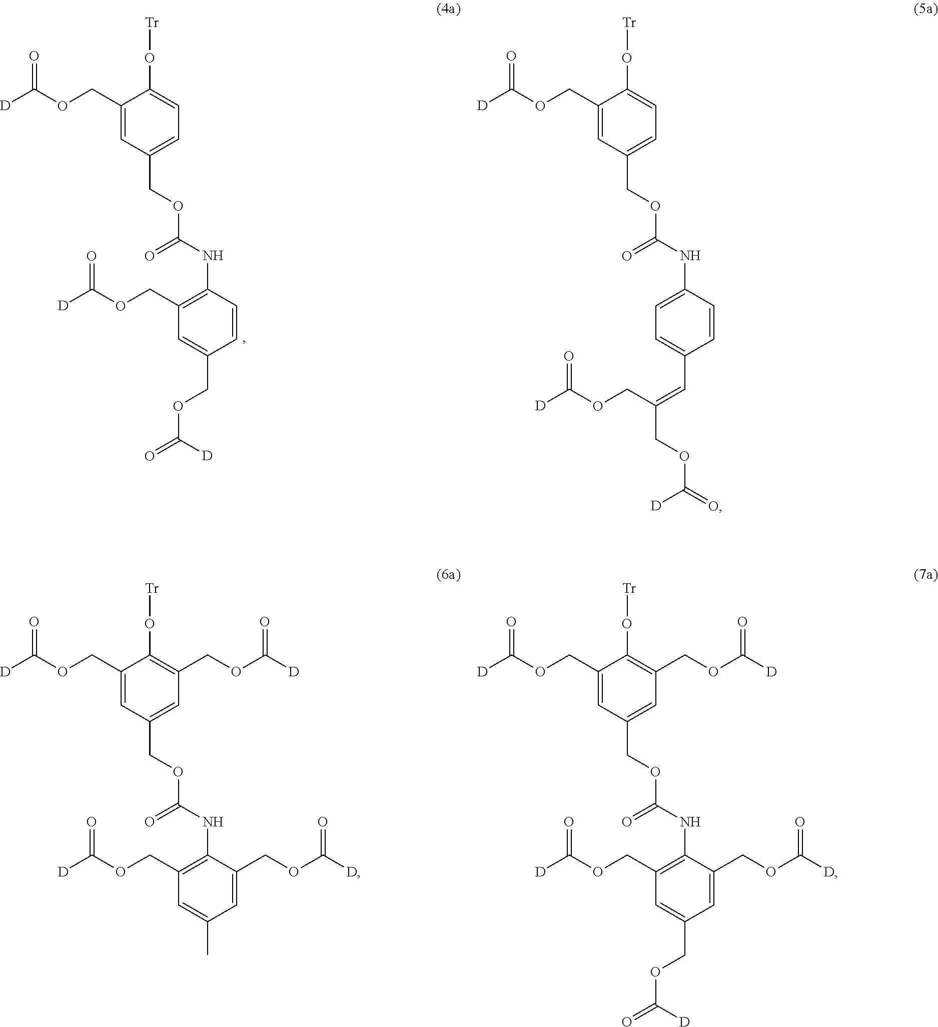

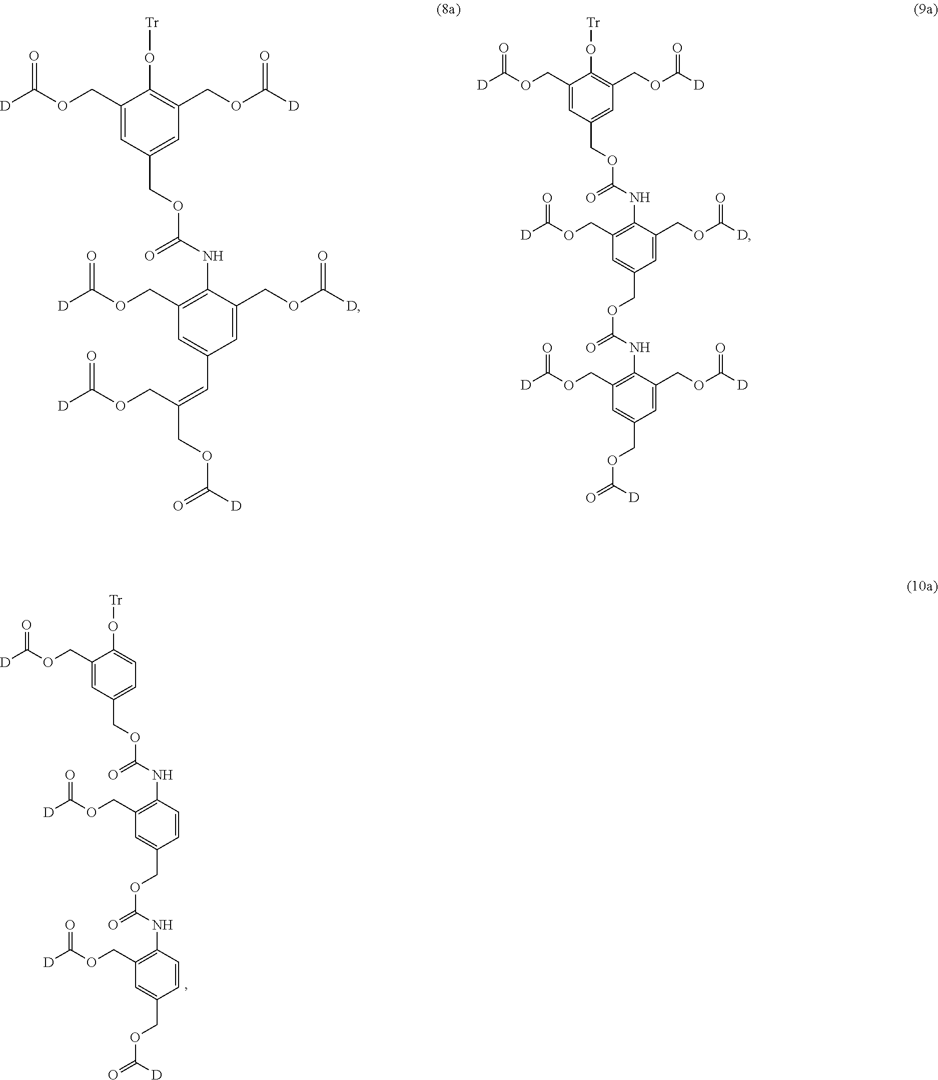

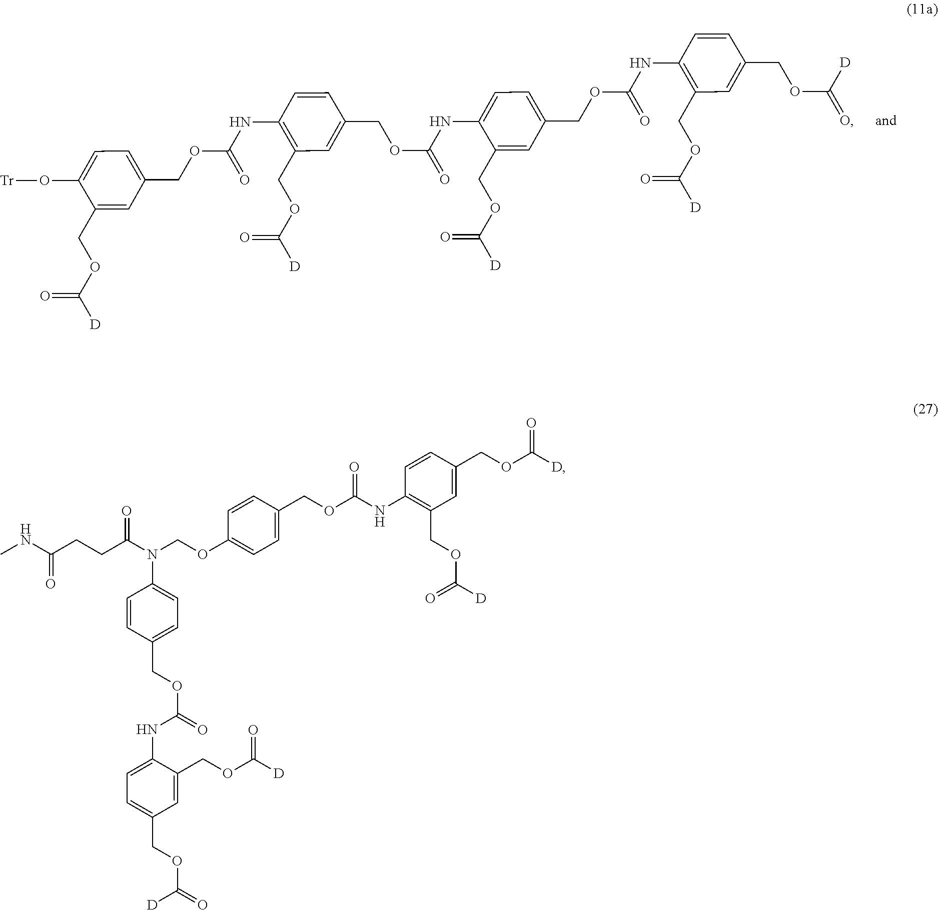

wherein each -D is independently a drug moiety comprising an albumin-binding moiety as described elsewhere herein and
-Tr is selected from the group consisting of

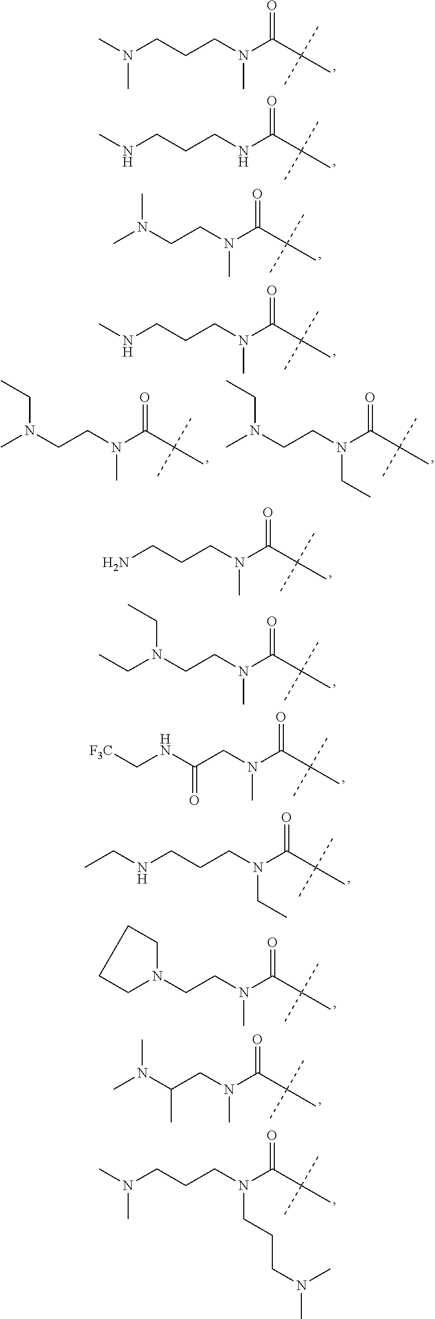

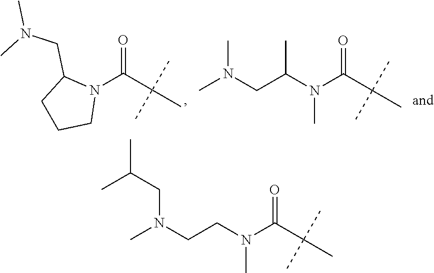

wherein the dashed line indicates the attachment to the oxygen atom.

The drug conjugate or pharmaceutically acceptable salt thereof according to the present invention may be of formula (IIc):

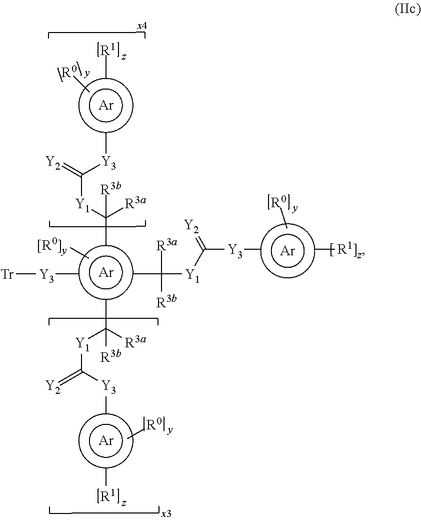

wherein
each —R is independently selected from the group consisting of

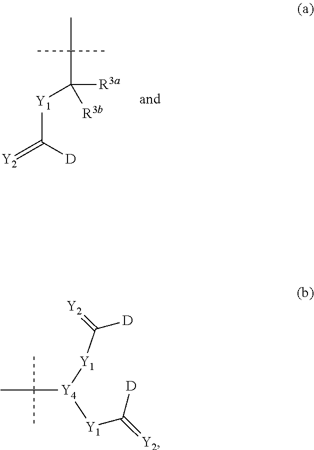

wherein the dashed line indicates the attachment to —Ar—;
each z is independently selected from the group consisting of 1, 2 and 3;
each y is independently selected from the group consisting of 0, 1, 2, 3, 4 and 5;
x3 and x4 are independently selected from the group consisting of 0, 1 and 2, provided that x3+x4≥1;
each -D is a drug moiety comprising an albumin-binding moiety as described elsewhere herein, wherein the total number of -Ds within the drug conjugate is ranging from 2 to 8;
-Tr is a cleavable triggering moiety as described elsewhere herein;
each —Y₁— is independently selected from the group consisting of —O— and —S—;
each ═Y₂ is independently selected from the group consisting of ═O and ═S;
each —Y₃— is independently selected from the group consisting of —O—, —S— and —N(R⁶)—;
each —Ar— is a ring independently selected from the group consisting of monocyclic or bicyclic aryl and heteroaryl, provided that —Ar— is connected to —Y₃— and —C(R^{3a})(R^{3b})—, via carbon atoms; wherein said monocyclic or bicyclic aryl and heteroaryl are optionally substituted with —R⁰, which are the same or different;
—Y₄— is

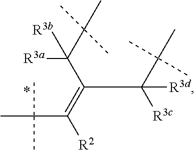

wherein the dashed line marked with the asterisk indicates the attachment to —Ar— and the unmarked dashed lines indicate the attachment to —Y₁—;
each —R⁰ is independently selected from the group consisting of —C(O)OH, -halogen, —NO₂, —CN, C₁₋₃₀ alkyl, C₂₋₃₀ alkenyl and C₂₋₃₀ alkynyl; wherein C₁₋₃₀ alkyl, C₂₋₃₀ alkenyl and C₂₋₃₀ alkynyl are optionally substituted with one or more —R⁴, which are the same or different; and wherein C₁₋₃₀ alkyl, C₂₋₃₀ alkenyl and C₂₋₃₀ alkynyl are optionally interrupted by one or more groups selected from the group consisting of -T-, —C(O)O—, —O—, —C(O)—, —C(O)N(R⁵)—, —S(O)₂N(R⁵)—, —S(O)N(R⁵)—, —S(O)₂—, —S(O)—, —N(R⁵)S(O)₂N(R^{5a})—, —S—, —N(R⁵)—, —OC(OR⁵)(R^{5a})—, —N(R⁵)C(O)N(R^{5a})— and —OC(O)N(R⁵)—;
each —R² is independently selected from the group consisting of —H, —C(O)OH, -halogen, —NO₂, —CN, C₁₋₃₀ alkyl, C₂₋₃₀ alkenyl and C₂₋₃₀ alkynyl; wherein C₁₋₃₀ alkyl, C₂₋₃₀ alkenyl and C₂₋₃₀ alkynyl are optionally substituted with one or more —R⁴, which are the same or different; and wherein C₁₋₃₀ alkyl, C₂₋₃₀ alkenyl and C₂₋₃₀ alkynyl are optionally interrupted by one or more groups selected from the group consisting of -T-, —C(O)O—, —O—, —C(O)—, —C(O)N(R⁵)—, —S(O)₂N(R⁵)—, —S(O)N(R⁵)—, —S(O)₂—, —S(O)—, —N(R⁵)S(O)₂N(R^{5a})—, —S—, —N(R⁵)—, —OC(OR⁵)(R^{5a})—, —N(R⁵)C(O)N(R^{5a})— and —OC(O)N(R⁵)—;
each —R^{3a}, R^{3b}, —R^{3c}, —R^{3d} are independently selected from the group consisting of —H, —C(O)OH, —F, —NO₂, —CN, C₁₋₃₀ alkyl, C₂₋₃₀ alkenyl and C₂₋₃₀ alkynyl; wherein C₁₋₃₀ alkyl, C₂₋₃₀ alkenyl and C₂₋₃₀ alkynyl are optionally substituted with one or more —R⁴, which are the same or different; and wherein C₁₋₃₀ alkyl, C₂₋₃₀ alkenyl and C₂₋₃₀ alkynyl are optionally interrupted by one or more groups selected from the group consisting of -T-, —C(O)O—, —O—, —C(O)—, —C(O)N(R⁵)—, —S(O)₂N(R⁵)—, —S(O)N(R⁵)—, —S(O)₂—, —S(O)—, —N(R⁵)S(O)₂N(R^{5a})—, —S—, —N(R⁵)—, —OC(OR⁵)(R^{5a})—, —N(R⁵)C(O)N(R^{5a})— and —OC(O)N(R⁵)—;
each -T- is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each -T- is independently optionally substituted with one or more —R⁴, which are the same or different; wherein —R⁴ is selected from the group consisting of —NO₂, —OCH₃, —CN, —N(R⁵)(R^{5a}), —OH, —C(O)OH and C₁₋₆ alkyl; wherein C₁₋₆ alkyl is optionally substituted with one or more halogen, which are the same or different;
—R⁵ and —R^{5a} are independently selected from the group consisting of —H and C₁₋₆ alkyl; wherein C₁₋₆ alkyl is optionally substituted with one or more halogen, which are the same or different; and
each —R⁶ is independently selected from the group consisting of —H, C₁₋₆ alkyl and —C(R^{3a})(R^{3b})—BB.

In certain embodiments, all —R¹ of formula (IIc) are

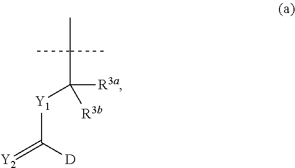

wherein —R^{3a}, —R^{3b}, —Y₁—, ═Y₂ and -D are as described elesewhere herein.

In certain embodiments, all —R¹ of formula (IIc) are

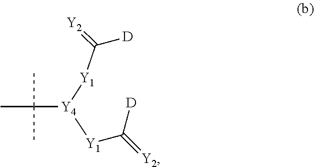

wherein —Y₄—, —Y₁, ═Y₂ and -D are as described elsewhere herein.

In certain embodiments, each z of formula (IIc) is 1. In certain embodiments, each z of formula (IIc) is 2. In certain embodiments, each z of formula (IIc) is 3.

In certain embodiments, each y of formula (IIc) is 0. In certain embodiments, each y of formula (IIc) is 1. In certain embodiments, each y of formula (IIc) is 2. In certain embodiments, each y of formula (IIc) is 3. In certain embodiments, each y of formula (IIc) is 4. In certain embodiments, each y of formula (IIc) is 5.

In certain embodiments, x3 and x4 of formula (IIc) are independently selected from the group consisting of 0 and 1, provided that x3+x4≥1. In certain embodiments, x3 is 0 and x4 is 1. In certain embodiments, both x3 and x4 are 1.

In certain embodiments, the total number of -Ds within the drug conjugate of formula (IIc) is 2. In certain embodiments, the total number of -Ds within the drug conjugate of formula (IIc) is 3. In certain embodiments, the total number of -Ds within the drug conjugate of formula (IIc) is 4. In certain embodiments, the total number of -Ds within the drug conjugate of formula (IIc) is 5. In certain embodiments, the total number of -Ds within the drug conjugate of formula (IIc) is 6. In certain embodiments, the total number of -Ds within the drug conjugate of formula (IIc) is 7. In certain embodiments, the total number of -Ds within the drug conjugate of formula (IIc) is 8.

In certain embodiments, each —Y₁— of formula (IIc) is —O—. In certain embodiments, each —Y₁— of formula (IIc) is —S—.

In certain embodiments, each ═Y₂ of formula (IIc) is ═O. In certain embodiments, each ═Y₂ of formula (IIc) is ═S.

In certain embodiments, each —Y₃— of formula (IIc) is —O—. In certain embodiments, each —Y₃— of formula (IIc) is —S—. In certain embodiments, each —Y₃— of formula (IIc) is —N(R⁶)—, wherein —R⁶ is as described elsewhere herein.

In certain embodiments, -Tr of formula (IIc) is

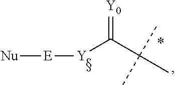

wherein the dashed line marked with an asterisk indicates the attachment to —Y₃—; and -Nu, -E, —Y_{§} — and ═Y₀ are as described elsewhere herein.

The drug conjugate or pharmaceutically acceptable salt thereof according to the present invention may be of formula (IIc1):

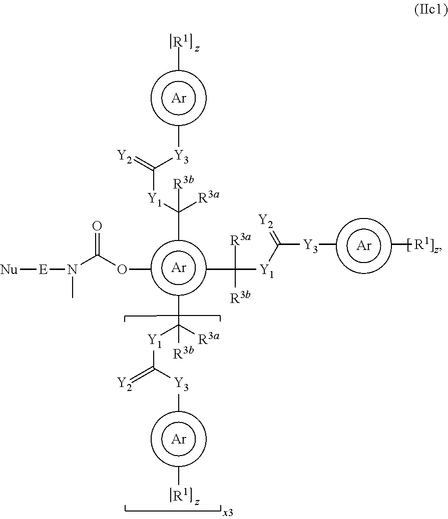

wherein
each —R¹ is independently selected from the group consisting of

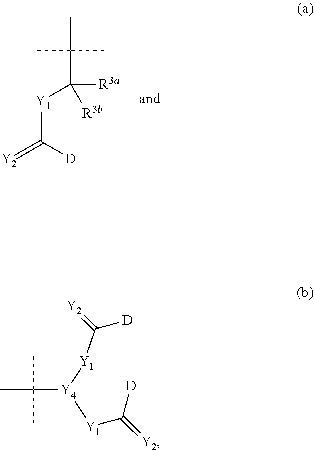

wherein the dashed line indicates the attachment to —Ar—;
each z is independently selected from the group consisting of 1, 2 and 3;
x3 is selected from the group consisting of 1 and 2;
each -D is a drug moiety comprising an albumin-binding moiety as described elsewhere herein, wherein the total number of -Ds within the drug conjugate is ranging from 2 to 8;
each —Y₁— is —O—;
each ═Y₂ is ═O;
each —Y₃— is independently selected from the group consisting of —O— and —N(R⁶)—;
-Nu, -E-, —Ar—, —Y₄—, —R^{3a}, —R^{3b} and —R⁶ are as described elsewhere herein;

In certain embodiments, the drug conjugate or pharmaceutically acceptable salt thereof of formula (IIc) is selected from the group consisting of

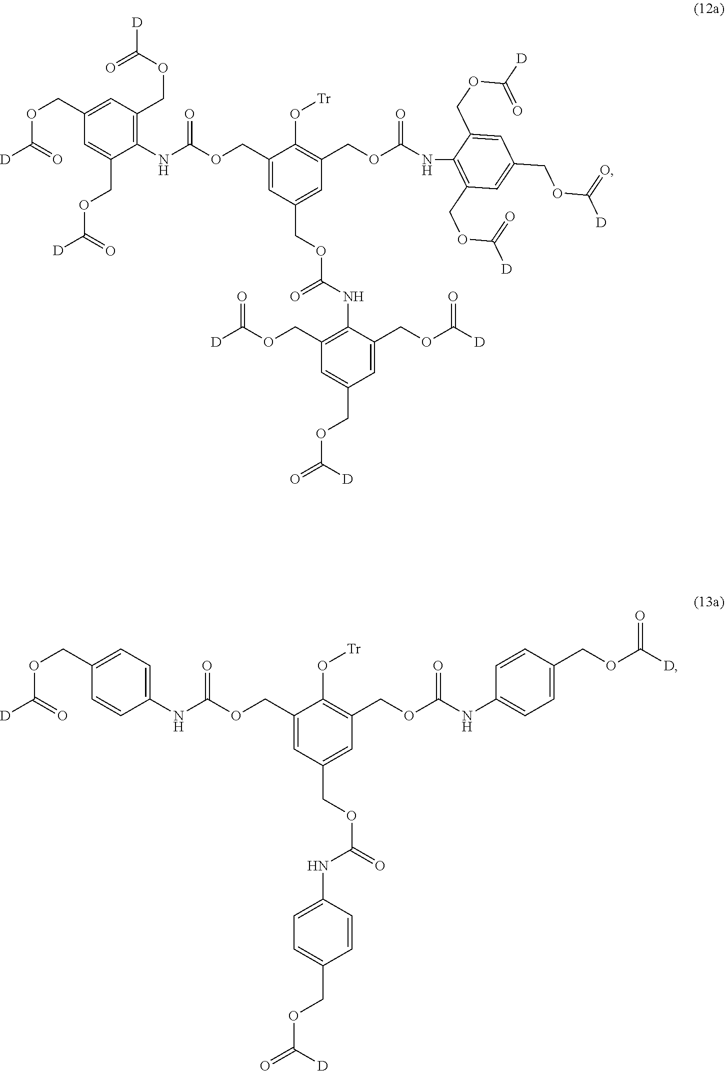

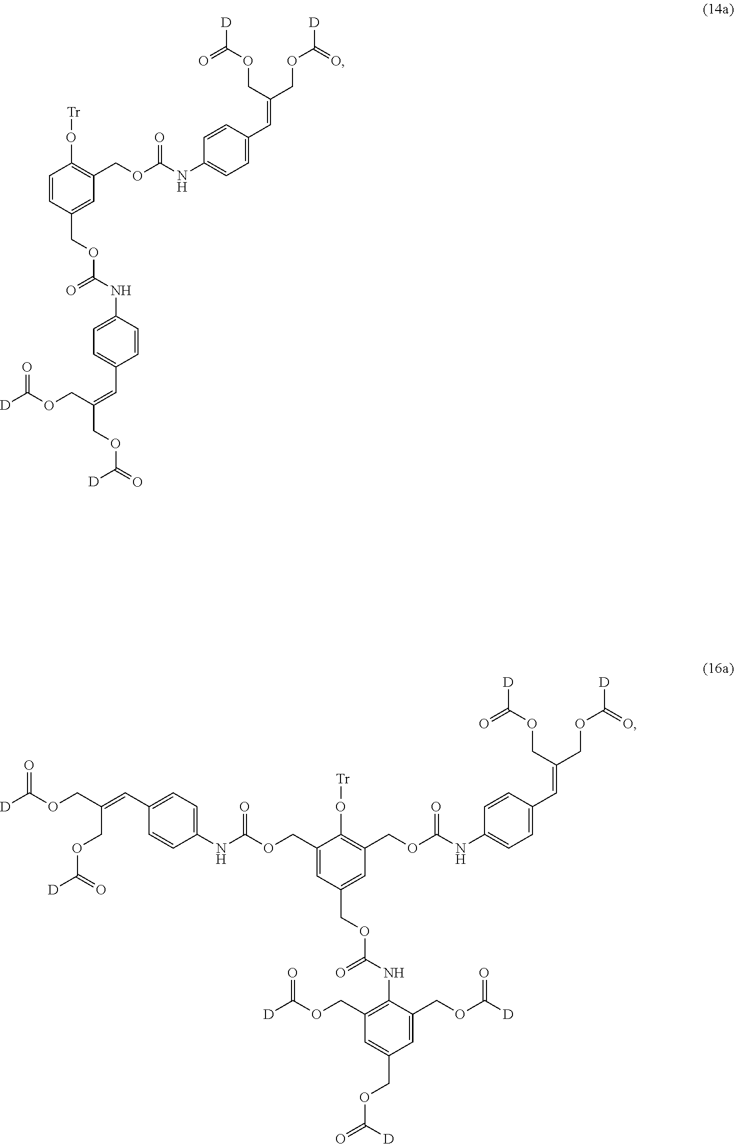

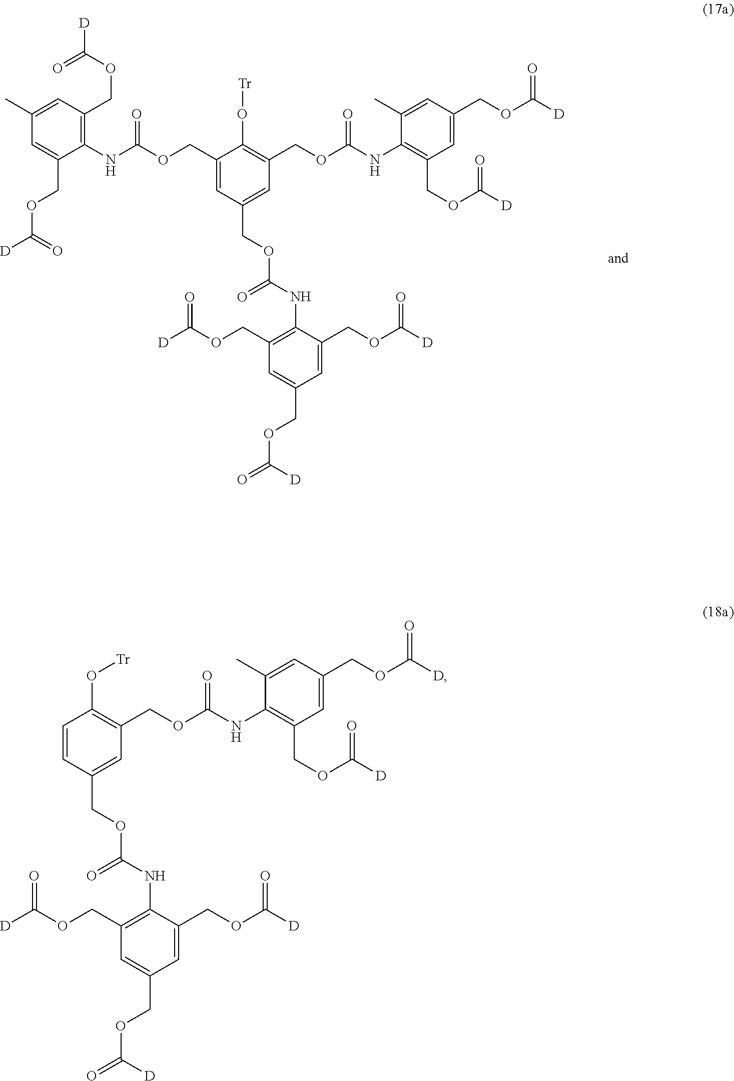

wherein each -D is independently a drug moiety comprising an albumin-binding moiety as described elsewhere herein and
-Tr is selected from the group consisting of

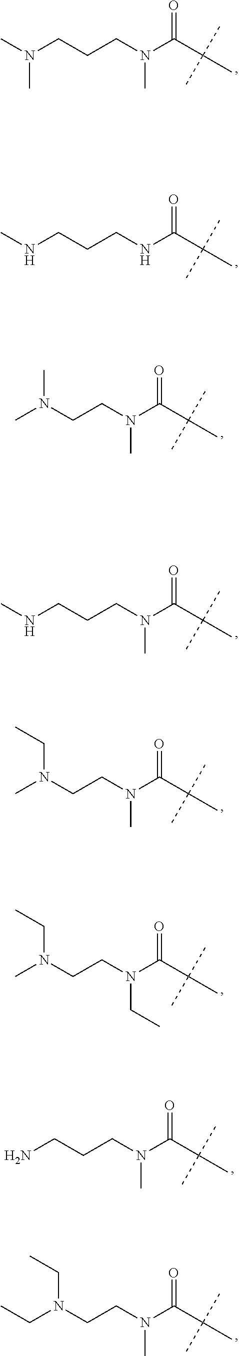

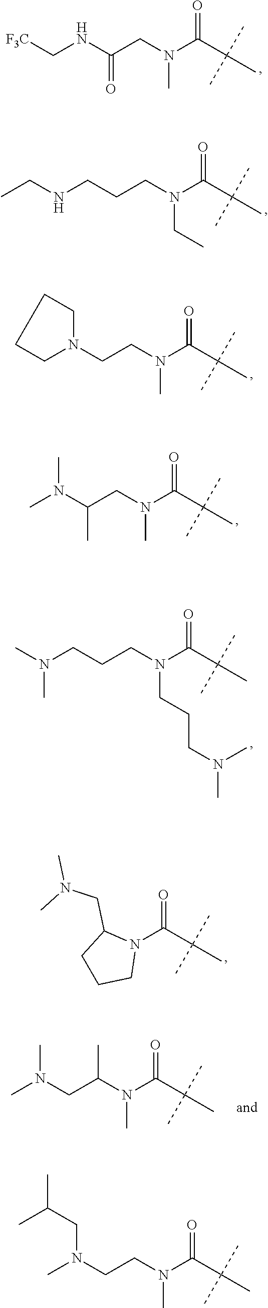

wherein the dashed line indicates the attachment to the oxygen atom.

The drug conjugate or pharmaceutically acceptable salt thereof of the present invention may be selected from the group consisting of

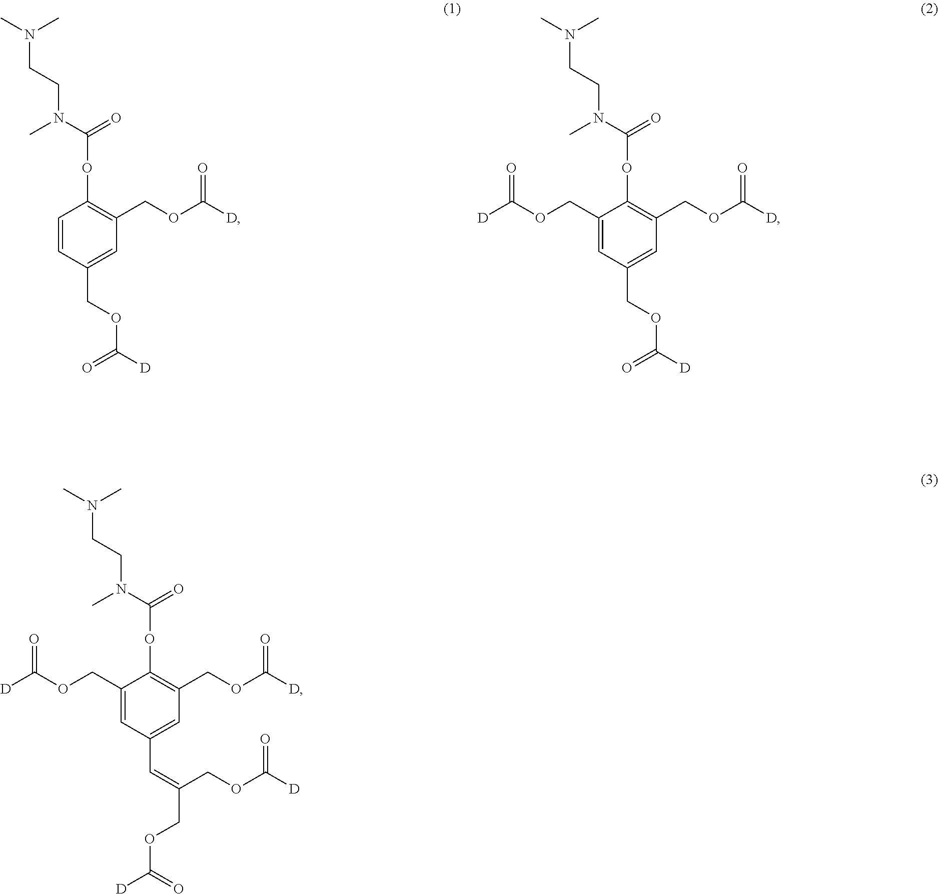

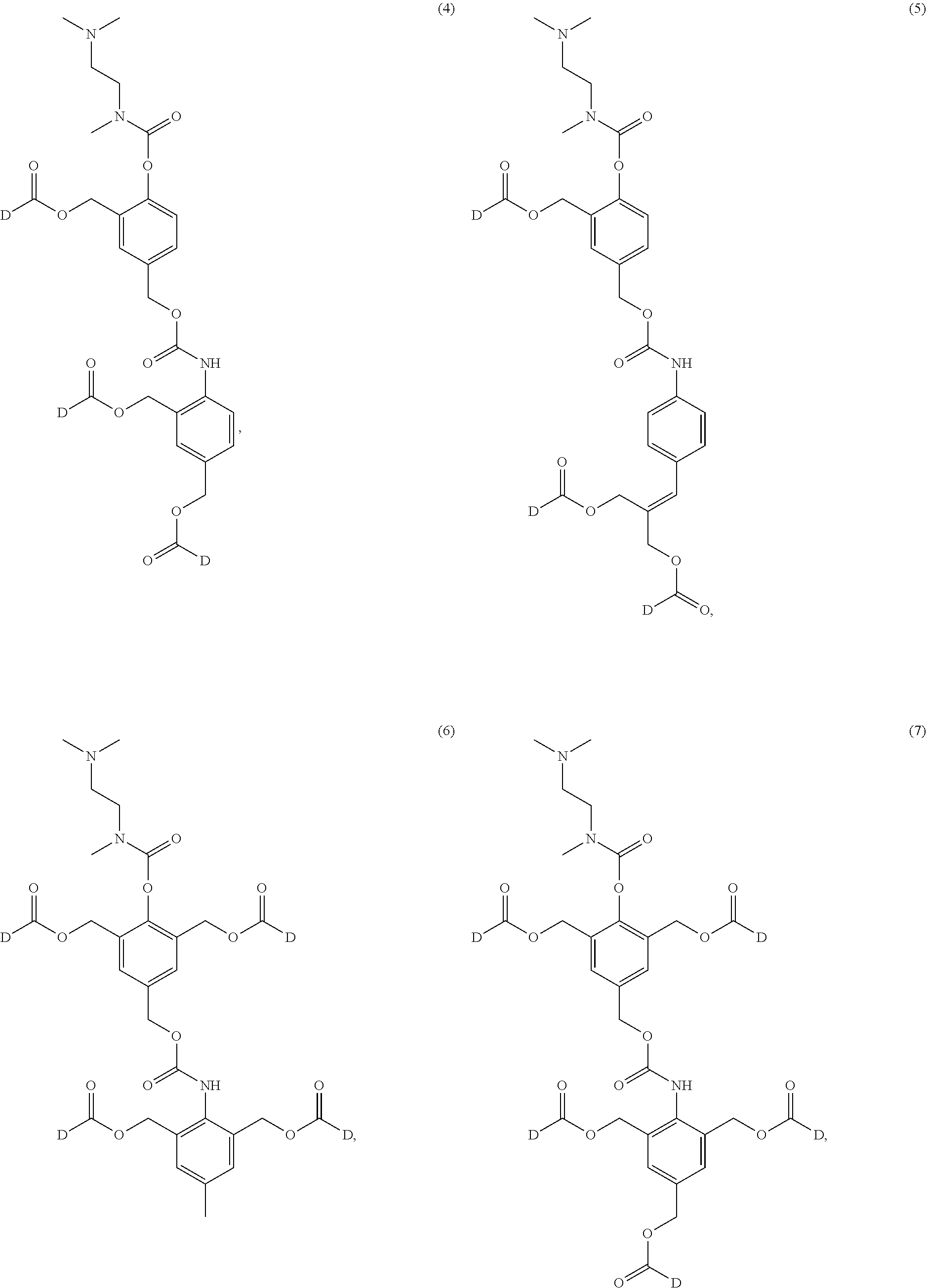

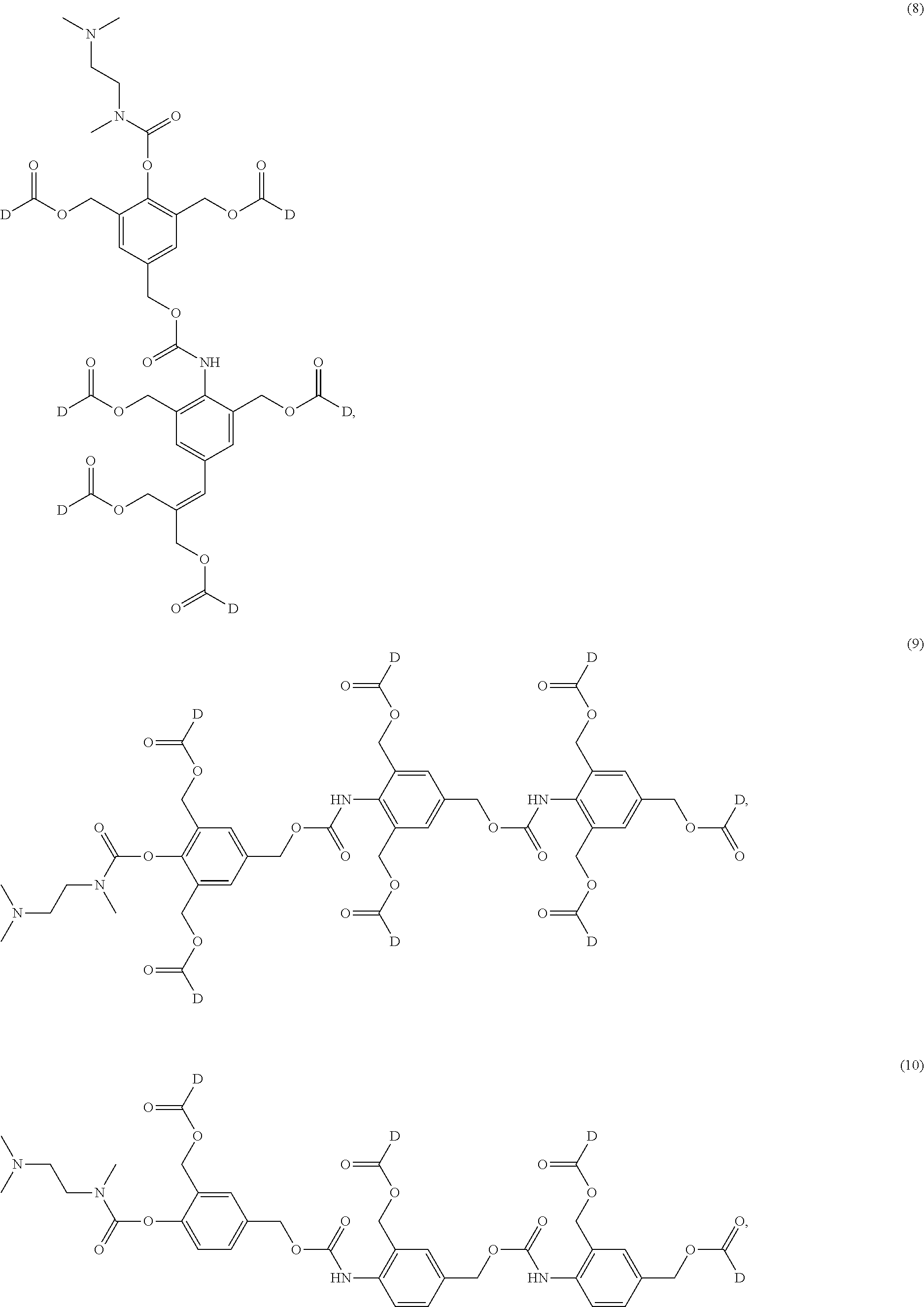

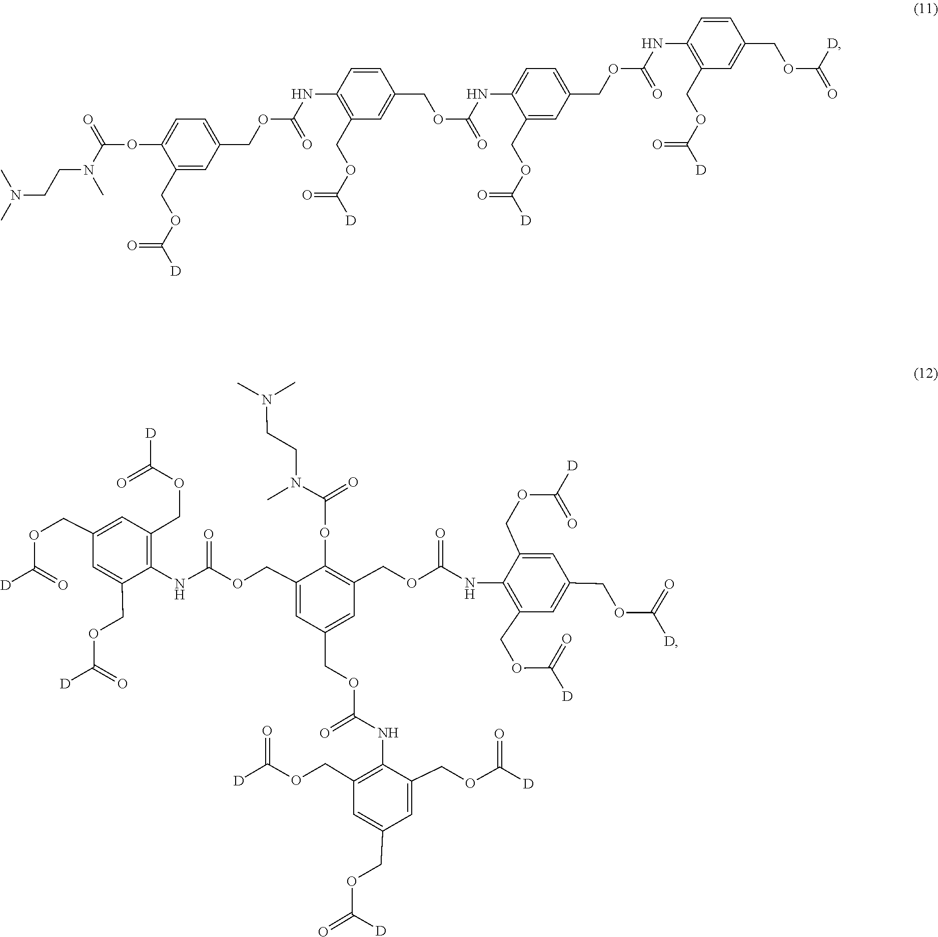

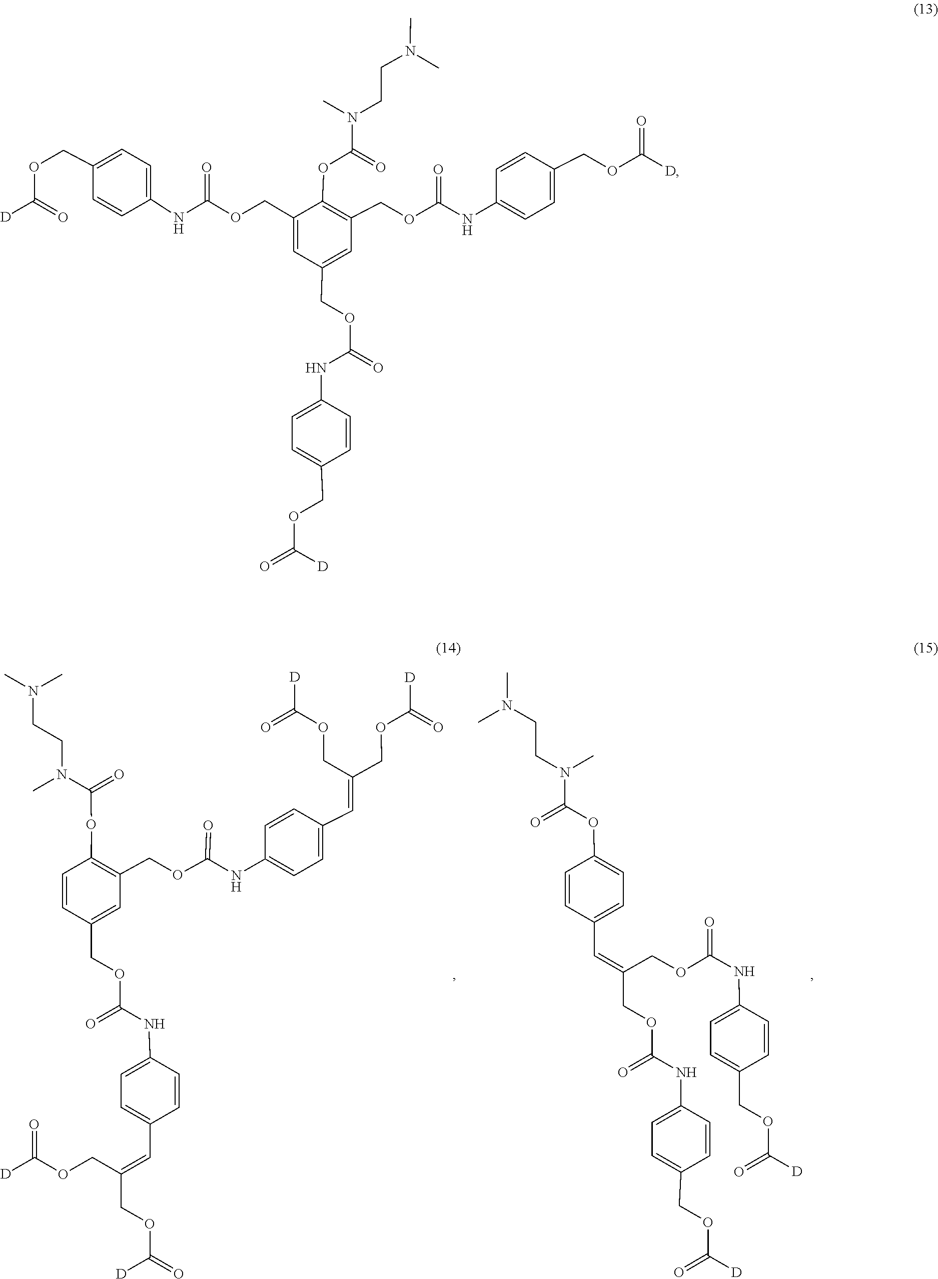

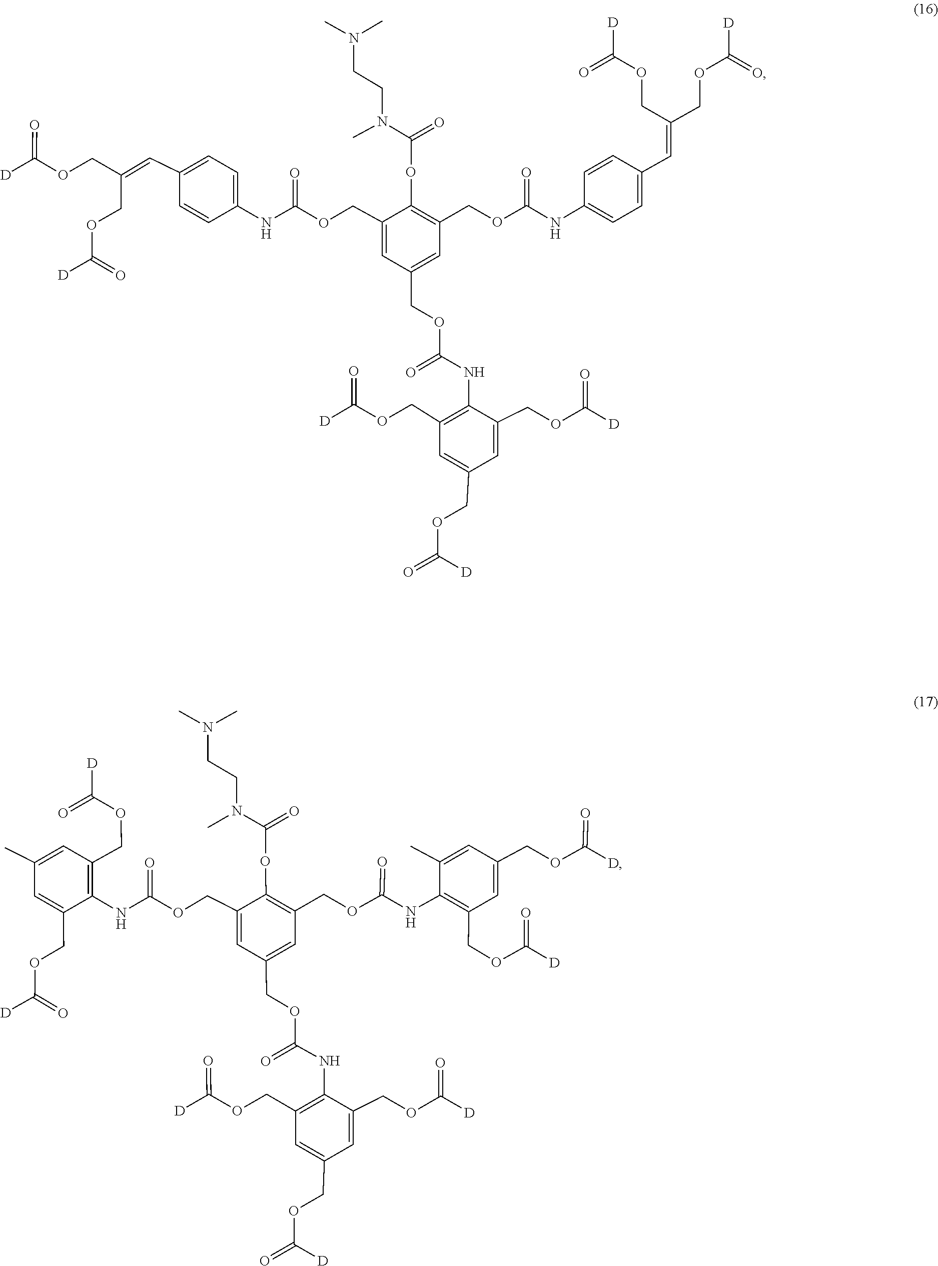

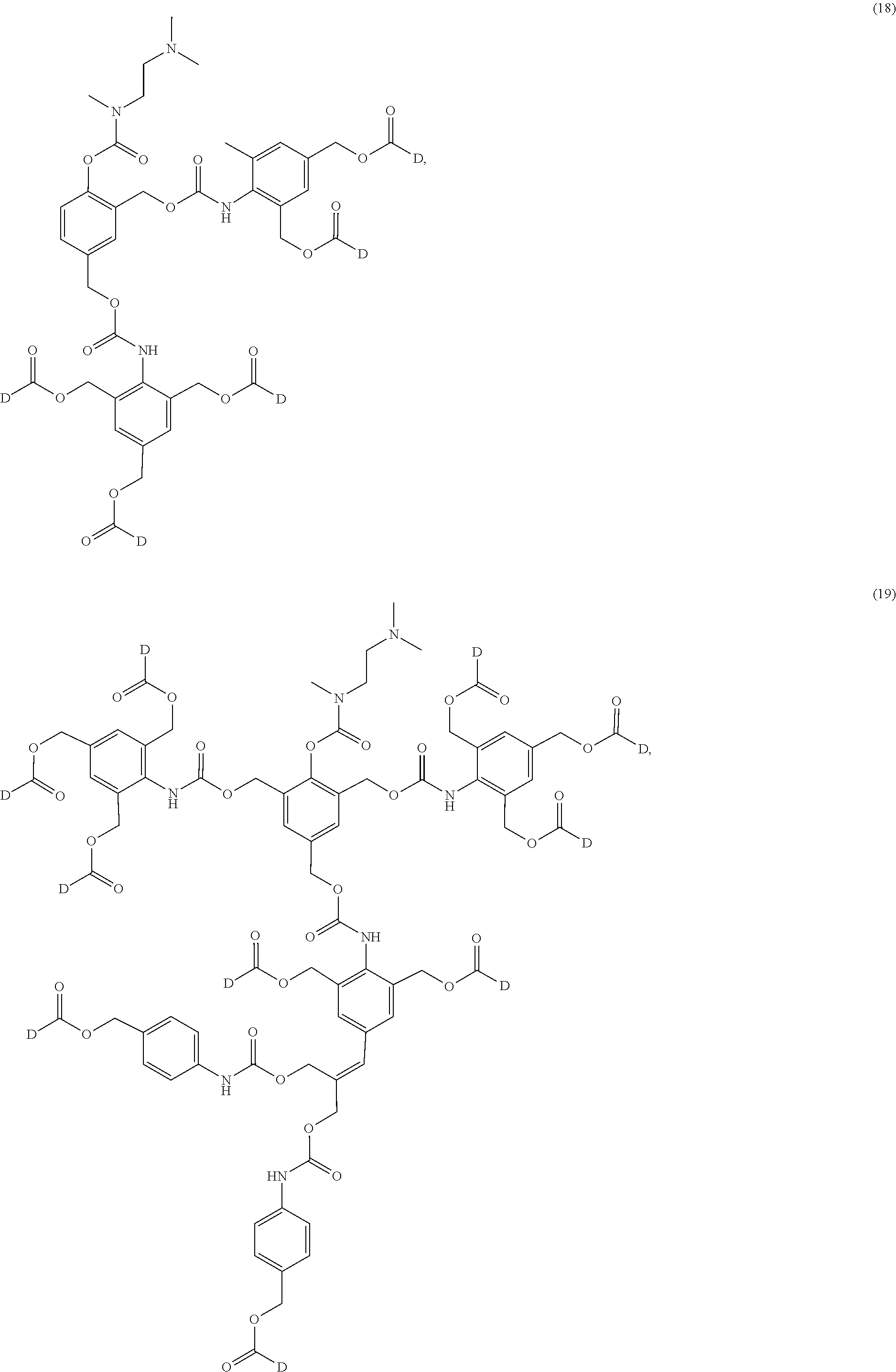

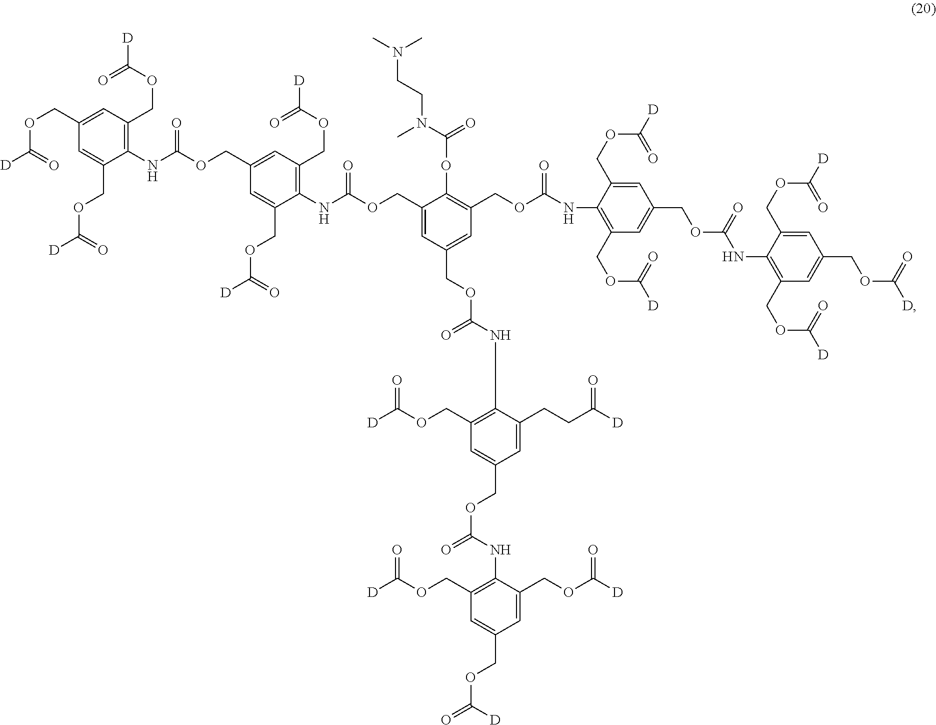

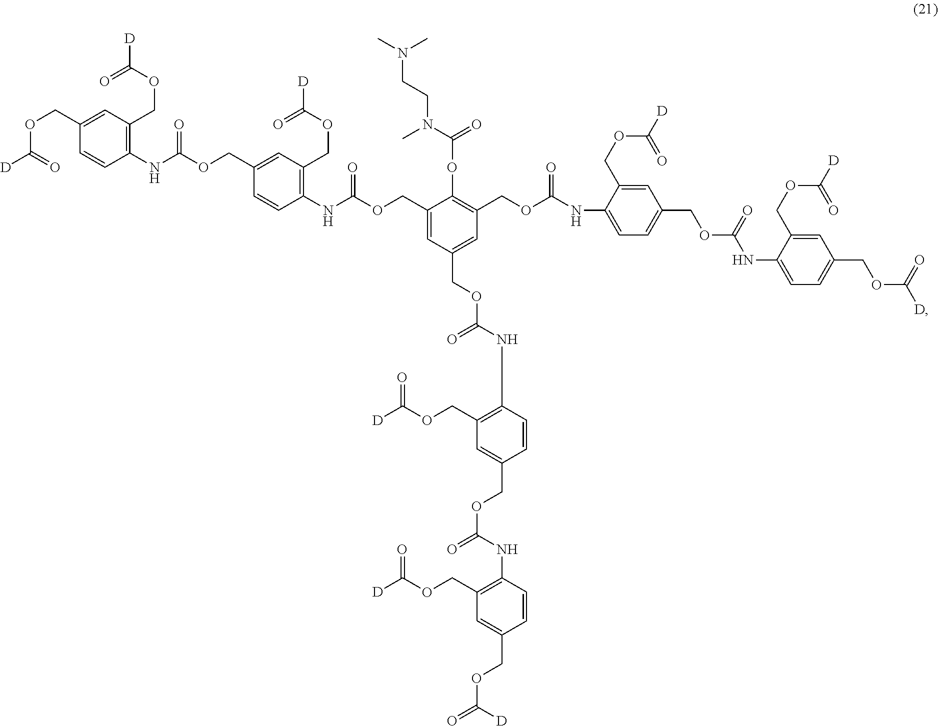

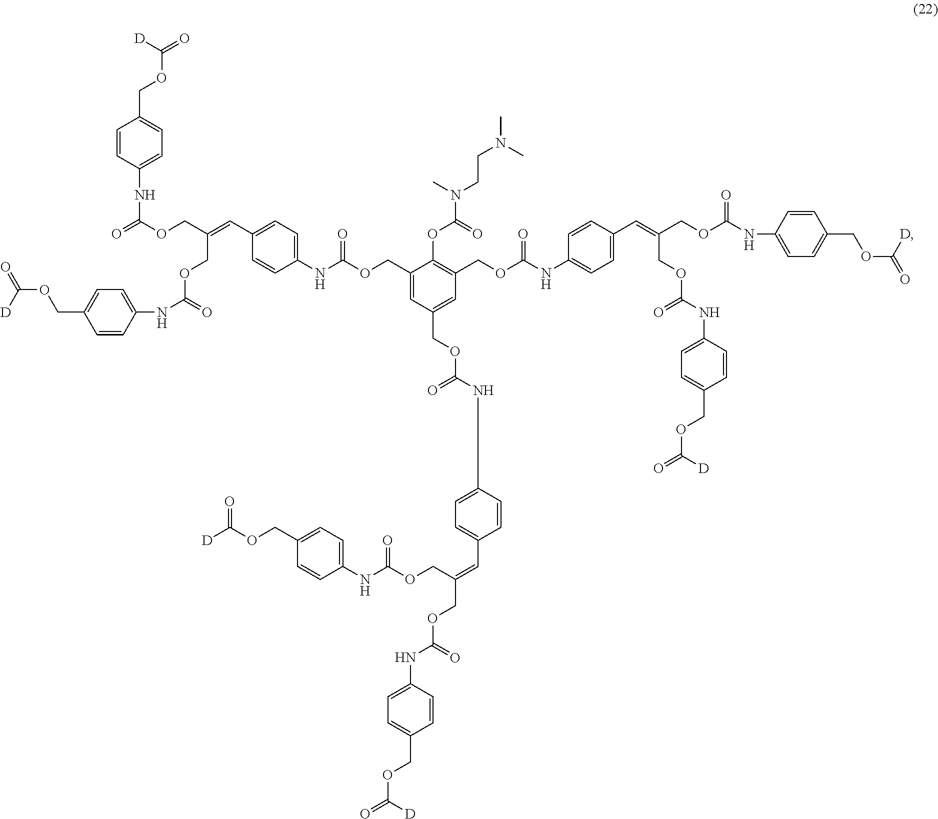

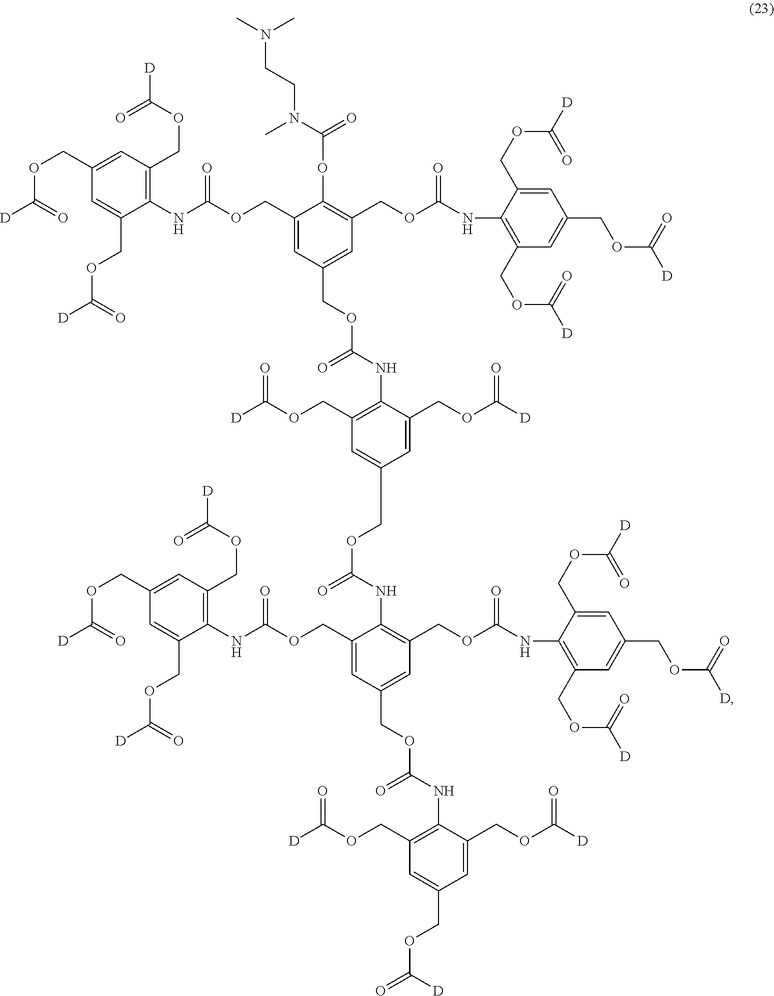

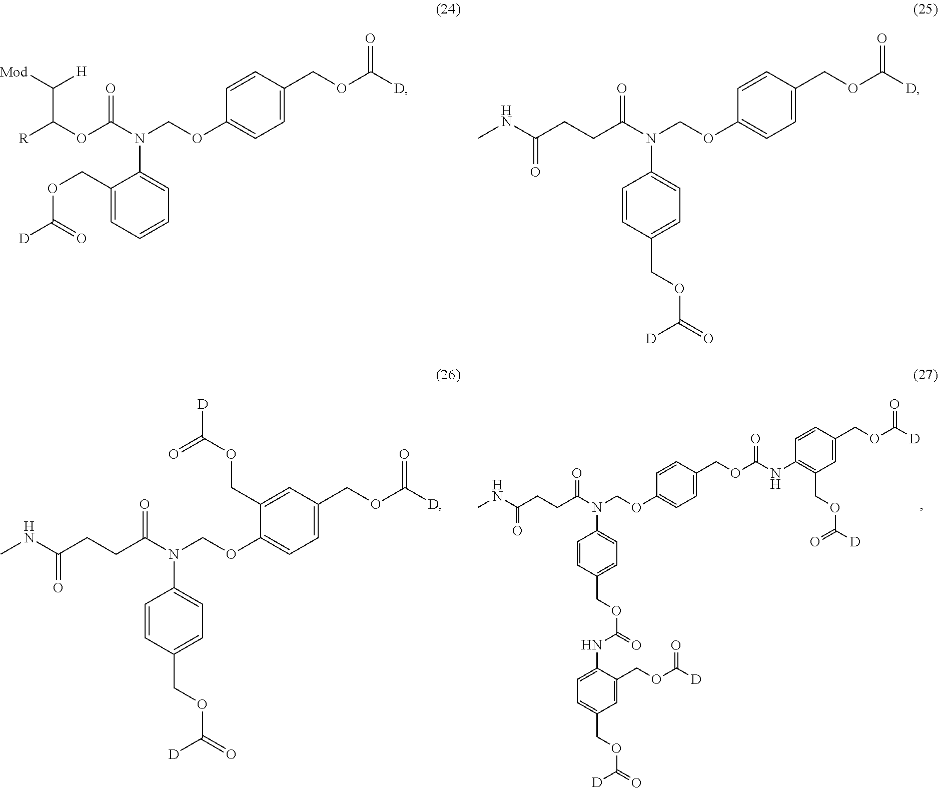

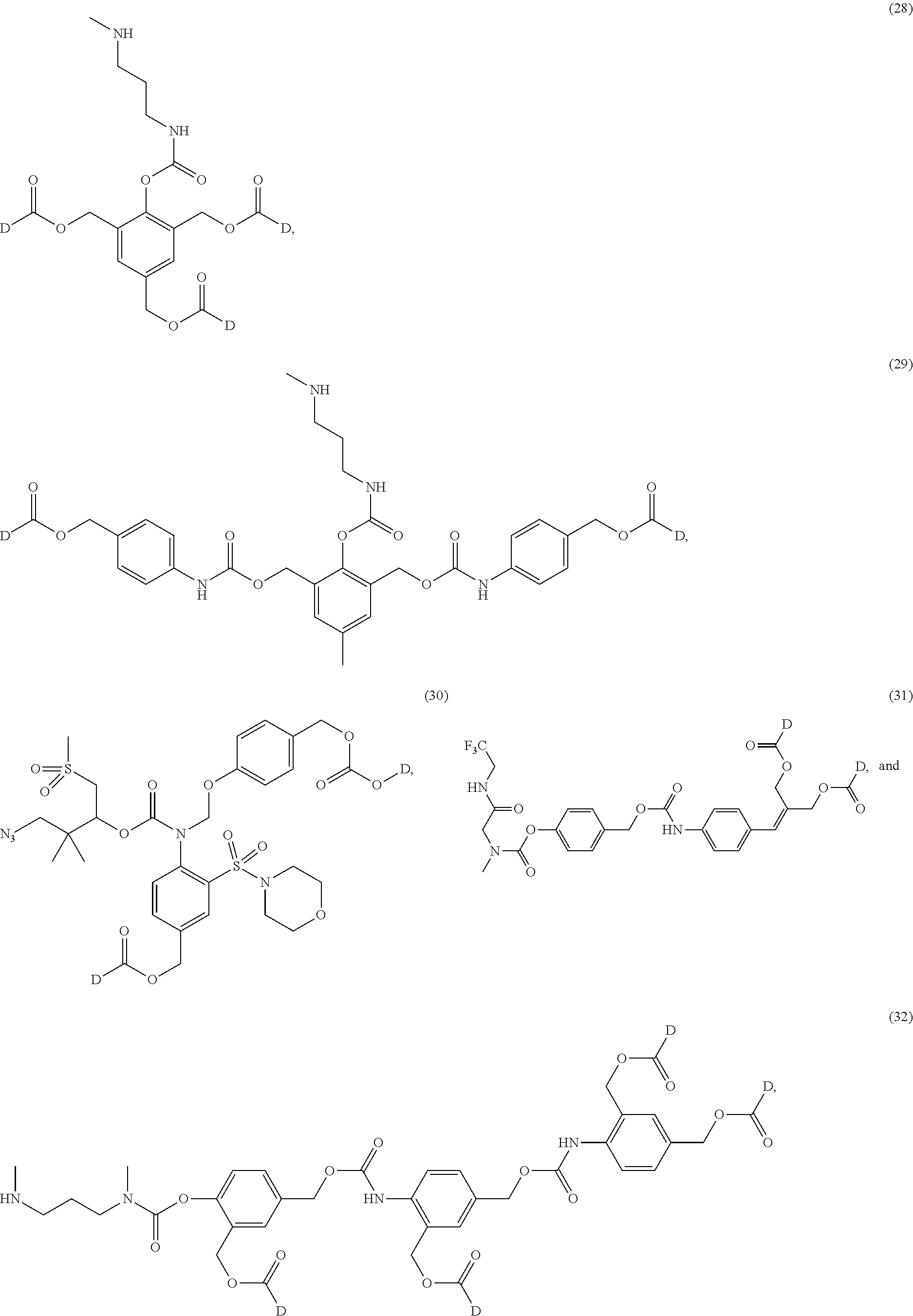

wherein each -D is independently a drug moiety comprising an albumin-binding moiety as described elsewhere herein; and
—R and -Mod are as described elsewhere herein.

In certain embodiments, the drug conjugate or pharmaceutically acceptable salt thereof of the present invention is selected from formulas (2), (4), (10), (11), (13), (14), (25), (28), (29), (30), (31) and (32).

In certain embodiments, the drug conjugate or pharmaceutically acceptable salt thereof of the present invention is of formula (1). In certain embodiments, the drug conjugate or pharmaceutically acceptable salt thereof of the present invention is of formula (2). In certain embodiments, the drug conjugate or pharmaceutically acceptable salt thereof of the present invention is of formula (3). In certain embodiments, the drug conjugate or pharmaceutically acceptable salt thereof of the present invention is of formula (4). In certain embodiments, the drug conjugate or pharmaceutically acceptable salt thereof of the present invention is of formula (5). In certain embodiments, the drug conjugate or pharmaceutically acceptable salt thereof of the present invention is of formula (6). In certain embodiments, the drug conjugate or pharmaceutically acceptable salt thereof of the present invention is of formula (7). In certain embodiments, the drug conjugate or pharmaceutically acceptable salt thereof of the present invention is of formula (8). In certain embodiments, the drug conjugate or pharmaceutically acceptable salt thereof of the present invention is of formula (9). In certain embodiments, the drug conjugate or pharmaceutically acceptable salt thereof of the present invention is of formula (10). In certain embodiments, the drug conjugate or pharmaceutically acceptable salt thereof of the present invention is of formula (11). In certain embodiments, the drug conjugate or pharmaceutically acceptable salt thereof of the present invention is of formula (12). In certain embodiments, the drug conjugate or pharmaceutically acceptable salt thereof of the present invention is of formula (13). In certain embodiments, the drug conjugate or pharmaceutically acceptable salt thereof of the present invention is of formula (14). In certain embodiments, the drug conjugate or pharmaceutically acceptable salt thereof of the present invention is of formula (15). In certain embodiments, the drug conjugate or pharmaceutically acceptable salt thereof of the present invention is of formula (16). In certain embodiments, the drug conjugate or pharmaceutically acceptable salt thereof of the present invention is of formula (17). In certain embodiments, the drug conjugate or pharmaceutically acceptable salt thereof of the present invention is of formula (18). In certain embodiments, the drug conjugate or pharmaceutically acceptable salt thereof of the present invention is of formula (19). In certain embodiments, the drug conjugate or pharmaceutically acceptable salt thereof of the present invention is of formula (20). In certain embodiments, the drug conjugate or pharmaceutically acceptable salt thereof of the present invention is of formula (21). In certain embodiments, the drug conjugate or pharmaceutically acceptable salt thereof of the present invention is of formula (22). In certain embodiments, the drug conjugate or pharmaceutically acceptable salt thereof of the present invention is of formula (23). In certain embodiments, the drug conjugate or pharmaceutically acceptable salt thereof of the present invention is of formula (24). In certain embodiments, the drug conjugate or pharmaceutically acceptable salt thereof of the present invention is of formula (25). In certain embodiments, the drug conjugate or pharmaceutically acceptable salt thereof of the present invention is of formula (26). In certain embodiments, the drug conjugate or pharmaceutically acceptable salt thereof of the present invention is of formula (27). In certain embodiments, the drug conjugate or pharmaceutically acceptable salt thereof of the present invention is of formula (28). In certain embodiments, the drug conjugate or pharmaceutically acceptable salt thereof of the present invention is of formula (29). In certain embodiments, the drug conjugate or pharmaceutically acceptable salt thereof of the present invention is of formula (30). In certain embodiments, the drug conjugate or pharmaceutically acceptable salt thereof of the present invention is of formula (31). In certain embodiments, the drug conjugate or pharmaceutically acceptable salt thereof of the present invention is of formula (32).

-D of the present invention may be of formula -D′-AB, wherein -AB is the albumin-binding moiety and -D′- is as defined in the following paragraphs.

It is understood that an albumin-binding moiety, such as -AB, binds to albumin, such as human albumin under physiological conditions (aqueous buffer, pH 7.4, 37° C.).

In certain embodiments, -D′- or -D′-AB is selected from the group consisting of small molecule drug moieties, medium size molecule drug moieties, oligonucleotide drug moieties, peptide nucleic acid drug moieties, peptide drug moieties and protein drug moieties.

In certain embodiments, -D′- or -D′-AB is a peptide drug moiety. In certain embodiments, -D′- or -D′-AB is a small molecule drug moiety. In certain embodiments, -D′- or -D′-AB is a medium size drug moiety. In certain embodiments, -D′- or -D′-AB is an oligonucleotide drug moiety. In certain embodiments, -D′- or -D′-AB is a peptide nucleic acid drug moiety. In certain embodiments, -D′- or -D′-AB is a protein drug moiety.

In certain embodiments, -D′- or -D′-AB is a GLP-1 receptor agonist. Accordingly, the conjugate of the present invention may be a GLP-1 receptor agonist conjugate.

As used herein, the term “GLP-1 receptor agonist” refers to agonists of the GLP-1 receptor (GLP1R) and optionally in addition agonists of one or more other receptors, such as for example a receptor for gastric inhibitory polypeptide (GIPR), a receptor for glucagon (GCGR), a receptor for amylin, a receptor for peptide YY (PYYR) or a receptor for glucagon-like peptide-2 (GLP2R). An “agonist” of a receptor, such as an agonist of the GLP-1 receptor, is a chemical compound that activates such receptor to produce a biological response. If a GLP-1 receptor agonist is in addition to being a GLPR1 agonist also an agonist of one receptor other than GLP1R, such as an agonist of GIPR, GCGR, the amylin receptor, PYYR or GLP2R, such GLP-1 receptor agonist is also referred to as being a “dual GLP-1 receptor agonist” or short “dual agonist”. Likewise, if a GLP-1 receptor agonist is in addition to being a GLPR1 agonist also an agonist of two other receptors, which may be selected from the group consisting of such as an agonist of GIPR, GCGR, the amylin receptor, PYYR or GLP2R, such GLP-1 receptor agonist is also referred to as being a “triple GLP-1 receptor agonist” or short “triple agonist”.

In certain embodiments, -D′- or -D′-AB is a mono agonist of the GLP-1 receptor, i.e., only activates the GLP-1 receptor.

In certain embodiments, -D′- or -D′-AB is an agonist of the GLP-1 receptor and an agonist of another receptor, i.e., -D′- or -D′-AB is a dual GLP-1 receptor agonist. Such other receptor may be selected from the group consisting of the GIP receptor, the GCG receptor, the amylin receptor, the PYY receptor and the GLP-2 receptor.

In certain embodiments, -D′- or -D′-AB is an agonist of the GLP-1 receptor and of the GIP receptor. In certain embodiments, -D′- or -D′-AB is an agonist of the GLP-1 receptor and of the GCG receptor. In certain embodiments -D′- or -D′-AB is an agonist of the GLP-1 receptor and of the amylin receptor. In certain embodiments, -D′- or -D′-AB is an agonist of the GLP-1 receptor and of the PYY receptor. In certain embodiments, -D′- or -D′-AB is an agonist of the GLP-1 receptor and of the GLP-2 receptor.

In certain embodiments, -D′- or -D′-AB is an agonist of the GLP-1 receptor and an agonist of two other receptors, i.e., -D′- or -D′-AB is a triple GLP-1 receptor agonist. These other receptors may be selected from the group consisting of the GIP receptor (GIPR), the GCG receptor (GCGR), the amylin receptor, the PYY receptor (PYYR) and the GLP-2 receptor (GLP2R).

In certain embodiments, -D′- or -D′-AB is a triple GLP-1 receptor agonist that activates the GLP-1 receptor, the GIP receptor and the GCG receptor. In certain embodiments, -D′- or -D′-AB is a triple GLP-1 receptor agonist that activates the GLP-1 receptor, the GIP receptor and the amylin receptor. In certain embodiments, -D′- or -D′-AB is a triple GLP-1 receptor agonist that activates the GLP-1 receptor, the GIP receptor and the PYY receptor. In certain embodiments, -D′- or -D′-AB is a triple GLP-1 receptor agonist that activates the GLP-1 receptor, the GIP receptor and the GLP-2 receptor. In certain embodiments, -D′- or -D′-AB is a triple GLP-1 receptor agonist that activates the GLP-1 receptor, the GCG receptor and the amylin receptor. In certain embodiments, -D′- or -D′-AB is a triple GLP-1 receptor agonist that activates the GLP-1 receptor, the GCG receptor and the PYY receptor. In certain embodiments, -D′- or -D′-AB is a triple GLP-1 receptor agonist that activates the GLP-1 receptor, the GCG receptor and the GLP-2 receptor. In certain embodiments, -D′- or -D′-AB is a triple GLP-1 receptor agonist that activates the GLP-1 receptor, the amylin receptor and the PYY receptor. In certain embodiments, -D′- or -D′-AB is a triple GLP-1 receptor agonist that activates the GLP-1 receptor, the amylin receptor and the GLP-2 receptor. In certain embodiments, -D′- or -D′-AB is a triple GLP-1 receptor agonist that activates the GLP-1 receptor, the PYY receptor and the GLP-2 receptor.

In certain embodiments -D′- is human GLP-1 of SE ID NO:1:

HAEGTFTSDVSSYLEGQAAKEFIAWLVKGRG.

In certain embodiments, -D′- comprises one or more amino acid changes compared to SEQ ID NO:1, such as amino acid additions, amino acid deletions and/or amino acid substitutions, which may be at the N-terminus, the C-terminus and/or at an internal site. In certain embodiments, such analog has a maximum of 3 amino acid changes compared to SEQ ID NO:1.

In certain embodiments, -D′- has the sequence

(SEQ ID NO: 2)
HX₁EGTFTSDVSSYLEGQAAKEFIAWLVRGRG,

wherein X₁ is 2-aminoisobutyric acid (Aib).

In certain embodiments, -D′- has the sequence

(SEQ ID NO: 3)
HX₁EGTFTSDVSSYLEGQAAKEFIAWLVRGRG,

wherein X₁ is Aib and the C-terminal glycine, i.e., the glycine at position 31, is amidated as a C-terminal primary amide.

In certain embodiments, -D′- is exenatide. Exenatide has the sequence

(SEQ ID NO: 4)
HGEGTFTSDLSKQMEEEAVRLFIEWLKNGGPSSGAPPPS.

In certain embodiments, -D′- has the sequence

(SEQ ID NO: 5)
HGEGTFTSDLSKQMEEEAVRLFIEWLKNGGPSSGAPPPS,

wherein the C-terminal serine, i.e., the serine at position 39, is amidated as a C-terminal primary amide.

In certain embodiments, -D′- is lixisenatide. Lixisenatide has the sequence

(SEQ ID NO: 6)
HGEGTFTSDLSKQMEEEAVRLFIEWLKNGGPSSGAPPSKKKKKK,

wherein the C-terminal lysine, i.e., the lysine at position 44, is amidated as a C-terminal primary amide.

In certain embodiments, -D′- has the sequence

(SEQ ID NO: 7)
HGEGTFTSDLSKQMEEEAVRLFIEWLKNGGPSSGAPPSKKKKKK.

In certain embodiments, -D′- has the sequence

(SEQ ID NO: 8)
HX₁EGTFTSDLSKQX₂EEEAVRLFIEWLKQGGPSSGAPPPC,

wherein X₁ is D-alanine and X₂ is norleucine (Nle).

In certain embodiments, -D′- has the sequence

(SEQ ID NO: 9)
HX₁EGTFTSDLSKQX₂EEEAVRLFIEWLKQGGPSSGAPPPC,

wherein X₁ is D-alanine, X₂ is Ne and the C-terminal cysteine, i.e., the cysteine at position 39, is amidated as a C-terminal primary amide.

In certain embodiments, -D′- is PEG-loxenatide. PEG-loxenatide has the sequence

(SEQ ID NO: 10)
HX₁EGTFTSDLSKQX₂EEEAVRLFIEWLKQGGPSSGAPPPC,

wherein X₁ is D-alanine; X₂ is Nle; the cysteine at position 39 is chemically modified through conjugation to the thiol group of the cysteine side-chain with

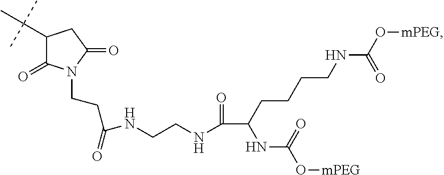

wherein
the dashed line indicates attachment to the thiol group of the cysteine side chain of the cysteine at position 39, and each mPEG is methoxypoly(ethylene glycol) with a molecular weight of approximately 20 kDa.

In certain embodiments, -D′- has the sequence

(SEQ ID NO: 11)
HX₁EGTFTSDLSKQX₂EEEAVRLFIEWLKQGGPSSGAPPPC,

wherein X₁ is D-alanine; X₂ is Nle; the cysteine at position 39 is chemically modified through conjugation to the thiol group of the cysteine side-chain with

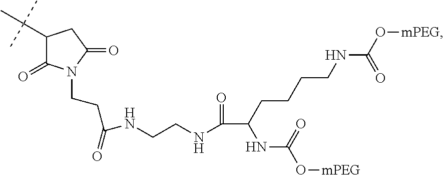

wherein
the dashed line indicates attachment to the thiol group of the cysteine side chain of the cysteine at position 39, each mPEG is methoxypoly(ethylene glycol) with a molecular weight of approx. 20 kDa, and the C-terminal cysteine, i.e., the cysteine at position 39, is amidated as a C-terminal primary amide.

In certain embodiments, -D′- has the sequence

(SEQ ID NO: 12)
HAEGTFTSDVSSYLEGQAAKEFIAWLVRGRG.

In certain embodiments, -D′- has the sequence

(SEQ ID NO: 13)
HAEGTFTSDVSSYLEGQAAKEFIAWLVRGRG,

and the C-terminal glycine, i.e., the glycine at position 31, is amidated as a C-terminal primary amide.

In certain embodiments, -D′- has the sequence

(SEQ ID NO: 14)
HVEGTFTSDVSSYLEEQAAREFIKWLVRGRG.

In certain embodiments, -D′- has the sequence

(SEQ ID NO: 15)
HVEGTFTSDVSSYLEEQAAREFIKWLVRGRG,

and the C-terminal glycine, i.e., the glycine at position 31, is amidated as a C-terminal primary amide.

In certain embodiments, -D′- has the sequence

(SEQ ID NO: 16)
HGEGTFTSDVSSYLEGQAAKEFIAWLVRGRG.

In certain embodiments, -D′- has the sequence

(SEQ ID NO: 17)
HGEGTFTSDVSSYLEGQAAKEFIAWLVRGRG,

and the C-terminal glycine, i.e., the glycine at position 31, is amidated as a C-terminal primary amide.

In certain embodiments, -D′- has the sequence

(SEQ ID NO: 18)
HX₁EGTFTSDVSSYLEGQAAKEFIAWLVRGRGL,

wherein X₁ is Aib.

In certain embodiments, -D′- has the sequence

(SEQ ID NO: 19)
HX₁EGTFTSDVSSYLEGQAAKEFIAWLVRGRGL,

wherein X₁ is Aib and the C-terminal leucine, i.e., the leucine at position 32, is amidated as a C-terminal primary amide.

In certain embodiments, -D′- has the sequence

(SEQ ID NO: 20)
YX₁EGTFTSDYSIX₂LDKIAQKAFVQWLIAGGPSSGAPPPS,

wherein X₁ is Aib, X₂ is Aib and the C-terminal serine, i.e., the serine at position 39, is amidated as a C-terminal primary amide.

In certain embodiments, -D′- has the sequence

(SEQ ID NO: 21)
YX₁EGTFTSDYSIX₂LDKIAQKAFVQWLIAGGPSSGAPPPS,

wherein X₁ is Aib and X₂ is Aib.

In certain embodiments, -D′- has the sequence

(SEQ ID NO: 22)
HSQGTFTSDKSEYLDSERARDFVAWLEAGG.

In certain embodiments, -D′- has the sequence

(SEQ ID NO: 23)
HSQGTFTSDKSEYLDSERARDFVAWLEAGG,

wherein the C-terminal glycine, i.e., the glycine at position 30, is amidated as a C-terminal primary amide.

In certain embodiments, -D′- has the sequence

(SEW ID NO: 24)
HX₁QGTFTSDYSKYLDERAAKDFIKWLESA,

wherein X₁ is 1-amino-cyclobutanecarboxylic acid (Ac4c); and the C-terminal alanine, i.e. the alanine at position 29, is amidated as a C-terminal primary amide.

In certain embodiments, -D′- has the sequence

(SEW ID NO: 25)
HX₁QGTFTSDYSKYLDERAAKDFIKWLESA,

wherein X₁ is 1-amino-cyclobutanecarboxylic acid (Ac4c).

In certain embodiments, -D′- has the sequence

(SEQ ID NO: 26)
HX₁QGTFTSDYSKYLDEKAAKEFIQWLLQT,

wherein X₁ is Aib and the glutamic acid at position 16 and the lysine at position 20 are connected via a lactam bridge.

In certain embodiments, -D′- has the sequence

(SEQ ID NO: 27)
HX₁QGTFTSDYSKYLDEKAAKEFIQWLLQT,

wherein X₁ is Aib, the glutamic acid at position 16 and the lysine at position 20 are connected via a lactam bridge and the C-terminal threonine, i.e., the threonine at position 29, is amidated as a C-terminal primary amide.

In certain embodiments, -D′- has the sequence

(SEQ ID NO: 28)
HX₁QGTFTSDYSKYLDEKKAKEFVEWLLEGGPSSG,

wherein X₁ is Aib and the C-terminal glycine, i.e., the glycine at position 34, is amidated as a C-terminal primary amide.

In certain embodiments, -D′- has the sequence

(SEQ ID NO: 29)
HX₁QGTFTSDYSKYLDEKKAKEFVEWLLEGGPSSG,

wherein X₁ is Aib.

(SEQ ID NO: 30)
HX₁EGSFTSELATILDKQAARDFIAWLIQHKITD,

In certain embodiments, -D′- has the sequence

(SEQ ID NO: 31)
HX₁EGSFTSELATILDKQAARDFIAWLIQHKITD,

wherein X₁ is Aib and the C-terminal aspartic acid, i.e., the aspartic acid at position 33, is amidated as a C-terminal primary amide.

In certain embodiments, -D′- has the sequence

(SEQ ID NO: 32)
YX₁QGTFTSDYSIX₂LDKKAQX₃AFIEYLLEGGPSSGAPPPS,

wherein X₁ is Aib, X₂ is α-methyl-leucine (MeL), X₃ is Aib; and the C-terminal serine, i.e. the serine at position 39, is amidated as a C-terminal primary amide.

In certain embodiments, -D′- has the sequence

(SEQ ID NO: 32)
YX₁QGTFTSDYSIX₂LDKKAQX₃AFIEYLLEGGPSSGAPPPS,

wherein X₁ is Aib, X₂ is α-methyl-leucine (αMeL) and X₃ is Aib.

In certain embodiments, -D′- is selected from the group consisting of insulins; amylin and amylin/calcitonin; PYY; GIP; MSH; C5a binders; GDF15; PCSK9 I; immune stimulants; urocortin2; MIC-1; IL-1R antagonists; leptin; gastrin; glucagon; exendin-4; GLP-1; GLP-2; and GIP.

In certain embodiments, -D′- is an insulin, such as insulin detemir, insulin degludec and insulin. In certain embodiments, -D′- is amylin and amylin/calcitonin, such as for example cagrilintide. In certain embodiments, -D′- is PYY, such as NNC0165-1875. In certain embodiments, -D′- is GIP. In certain embodiments, -D′- is MSH. In certain embodiments, -D′- is a C5a binder, such as zilucoplan. In certain embodiments, -D′- is GDF15, such as NN LA-GDF15. In certain embodiments, -D′- is PCSK9 i. In certain embodiments, -D′- is an immune stimulant, such as romurtide or mifamurtide. In certain embodiments, -D′- is muramyl dipeptide. In certain embodiments, -D′- is urocortin2. In certain embodiments, -D′- is MIC-1. In certain embodiments, -D′- is an IL-1R antagonist. In certain embodiments, -D′- is leptin. In certain embodiments, -D′- is gastrin.

In certain embodiments, -D′- is selected from the list consisting of growth hormones, such as human growth hormone; FGF21; EGF(a); and coagulations factors.

In certain embodiments, -D′- is a growth hormone, such as a human growth hormone, such as somapacitan. In certain embodiments, -D′- is FGF21, such as NNC0194 0499. In certain embodiments, -D′- is EGF(a). In certain embodiments, -D′- is a coagulation factor.

In certain embodiments, -D′- is selected from cytotoxic small molecule drugs, chemotherapy small molecule drugs and immune activating small molecule drugs.

In certain embodiments, -D′- is a cytotoxic small molecule drug. In certain embodiments, -D′- is a chemotherapy small molecule drug. In certain embodiments, -D′- is and immune activating small molecule drug, such as telratolimod.

In certain embodiments, -D′- is paclitaxel. In certain embodiments, -D′- is doxorubicin. In certain embodiments, -D′- is 5-FU.

In certain embodiments, -D′- is PTH.

In certain embodiments, -AB is a fatty acid-based albumin-binding moiety.

In certain embodiments, -AB is of formula (A):

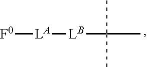

wherein
the dashed line indicates attachment to -D′-;
—F⁰ is of formula (a-1)

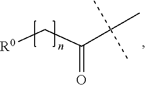

wherein
the dashed line indicates attachment to -L^{A}-;
—R⁰ is selected from the group consisting of —CR¹R^{1a}R^{1b}, —COOR¹,

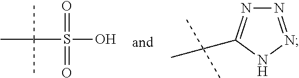

—R¹, —R^{1a} and —R^{1b} are selected from the group consisting of —H, methyl, ethyl, propyl, isopropyl;
n is an integer ranging from and including 8, 9, 10, 11, 12, 13, 14, 15, 16, 17, 18, 19, 20, 21 and 22;
-L^{A}- is absent or is of formula (a-2)

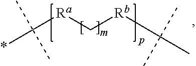

wherein
the unmarked dashed line indicates attachment to -L^{B}-;
the dashed line marked with the asterisk indicates attachment to —F⁰;
—R^{a}— is selected from the group consisting of

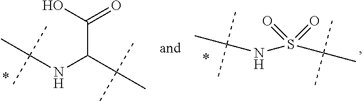

wherein
the dashed line marked with the asterisk indicates attachment to —F⁰;
the unmarked dashed line indicates attachment to the remainder of -L^{A}-;
—R^{b}— is

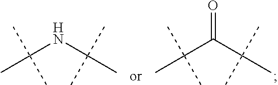

m is an integer selected from the group consisting of 1, 2, 3, 4, 5, 6, 7, 8, 9 and
p is an integer selected from the group consisting of 1, 2, 3, 4, 5, 6, 7, 8, 9 and 10;
-L^{B}- is absent or is of formula (a-3)

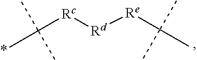

wherein
the unmarked dashed line indicates attachment to -D-;
the dashed line marked with the asterisk indicates attachment to -L^{A};
—R^{c}— is or

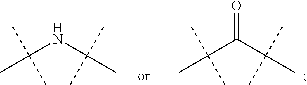

—R^{d}— is selected from the group consisting of C₁₋₅₀ alkyl, C₂₋₅₀ alkenyl or C₂₋₅₀ alkynyl, wherein C₁₋₅₀ alkyl, C₂₋₅₀ alkenyl, and C₂₋₅₀ alkynyl may be substituted with one or more —R¹, which may be the same or different, and which C₁₋₅₀ alkyl, C₂₋₅₀ alkenyl or C₂₋₅₀ alkynyl may be interrupted by one or more groups selected from the group consisting of -T-, —C(O)O—, —O—, —C(O)—, —C(O)N(R²)—, —S(O)₂N(R²)—, —S(O)N(R²)—, —S(O)₂—, —S(O)—, —N(R²)S(O)₂N(R^{2a})—, —S—, —N(R²)—, —OC(OR²)(R^{2a})—, —N(R²)C(O)N(R^{2a})—, and —OC(O)N(R²)—;
   each -T- is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each -T- may independently be substituted with one or more —R¹, which may be the same or different;
each —R¹ is independently selected from the group consisting of halogen, —CN, oxo (═O), —COOR³, —OR³, —C(O)R³, —C(O)N(R³R^{3a}), —S(O)₂N(R³R^{3a}), —S(O)N (R³R^{3a}), —S(O)₂R³, —S(O)R³, —N(R³)S(O)₂N(R^{3a}R^{3b}), —SR³,—N(R³R^{3a}), —NO₂, —OC(O)R³, —N(R³)C(O)R^{3a}, —N(R³)S(O)₂R^{3a}, —N(R³)S(O)R^{3a}, —N(R³)C(O)OR^{3a}, —N(R³)C(O)N(R^{3a}R^{3b}), —OC(O)N(R³R^{3a}), and C₁₋₆ alkyl; wherein C₁₋₆ alkyl is optionally substituted with one or more halogen, which are the same or different;
each —R², —R^{2a}, —R³, —R^{3a} and —R^{3b} is independently selected from the group consisting of —H, and C₁₋₆ alkyl, wherein C₁₋₆ alkyl may be substituted with one or more halogen, which may be the same or different; and
—R^{e}— is selected from the group consisting of —CH₂—,

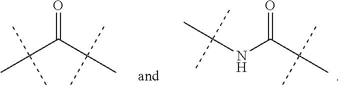

If —R^{b}— is

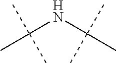

and -L^{B}- is not absent, then —R^{c}— is

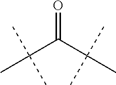

### and if —R^{b}— is

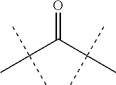

and -L^{B}- is not absent, then —R^{c}— is

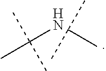

In certain embodiments, -L^{A}- is of formula a-2). In certain embodiments, -L^{A}- is absent.

In certain embodiments, —R^{a}— is

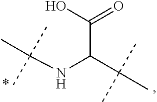

wherein the dashed line marked with the asterisk indicates attachment to —F⁰ and the unmarked dashed line indicates attachment to the remainder of -L^{A}.

In certain embodiments, —R^{a}— is

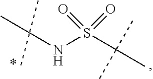

wherein the dashed line marked with the asterisk indicates attachment to —F⁰ and the unmarked dashed line indicates attachment to the remainder of -L^{A}-.

In certain embodiments, —R^{b}— is

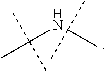

In certain embodiments, —R^{b}— is

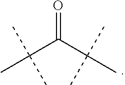

In certain embodiments, m of formula (a-2) is 1. In certain embodiments, m of formula (a-2) is 2. In certain embodiments, m of formula (a-2) is 3. In certain embodiments, m of formula (a-2) is 4. In certain embodiments, m of formula (a-2) is 5. In certain embodiments, m of formula (a-2) is 6. In certain embodiments, m of formula (a-2) is 7. In certain embodiments, m of formula (a-2) is 8. In certain embodiments, m of formula (a-2) is 9. In certain embodiments, m of formula (a-2) is 10.

In certain embodiments, p of formula (a-2) is 1. In certain embodiments, p of formula (a-2) is 2. In certain embodiments, p of formula (a-2) is 3. In certain embodiments, p of formula (a-2) is 4. In certain embodiments, p of formula (a-2) is 5. In certain embodiments, p of formula (a-2) is 6. In certain embodiments, p of formula (a-2) is 7. In certain embodiments, p of formula (a-2) is 8. In certain embodiments, p of formula (a-2) is 9. In certain embodiments, p of formula (a-2) is 10.

In certain embodiments, -L^{B}- is of formula (a-3). In certain embodiments, -L^{B}_is absent. If -L^{B}- is absent, the unmarked dashed line in formula (a-2) indicates attachment to -D-.

In certain embodiments, —R^{c}— is

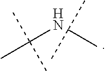

In certain embodiments, —R^{c}— is

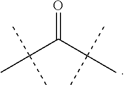

In certain embodiments, —R^{e}— is —CH₂—. In certain embodiments, —R^{e}— is

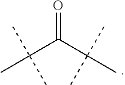

In certain embodiments, —R^{e}— is

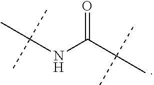

In certain embodiments, both -L^{A}- and -L^{B}- are absent. If both -L^{A}- and -L^{B}- are absent, the dashed line in formula (a-1) indicates attachment to -D-.

In certain embodiments, —F⁰ is selected from the group consisting of

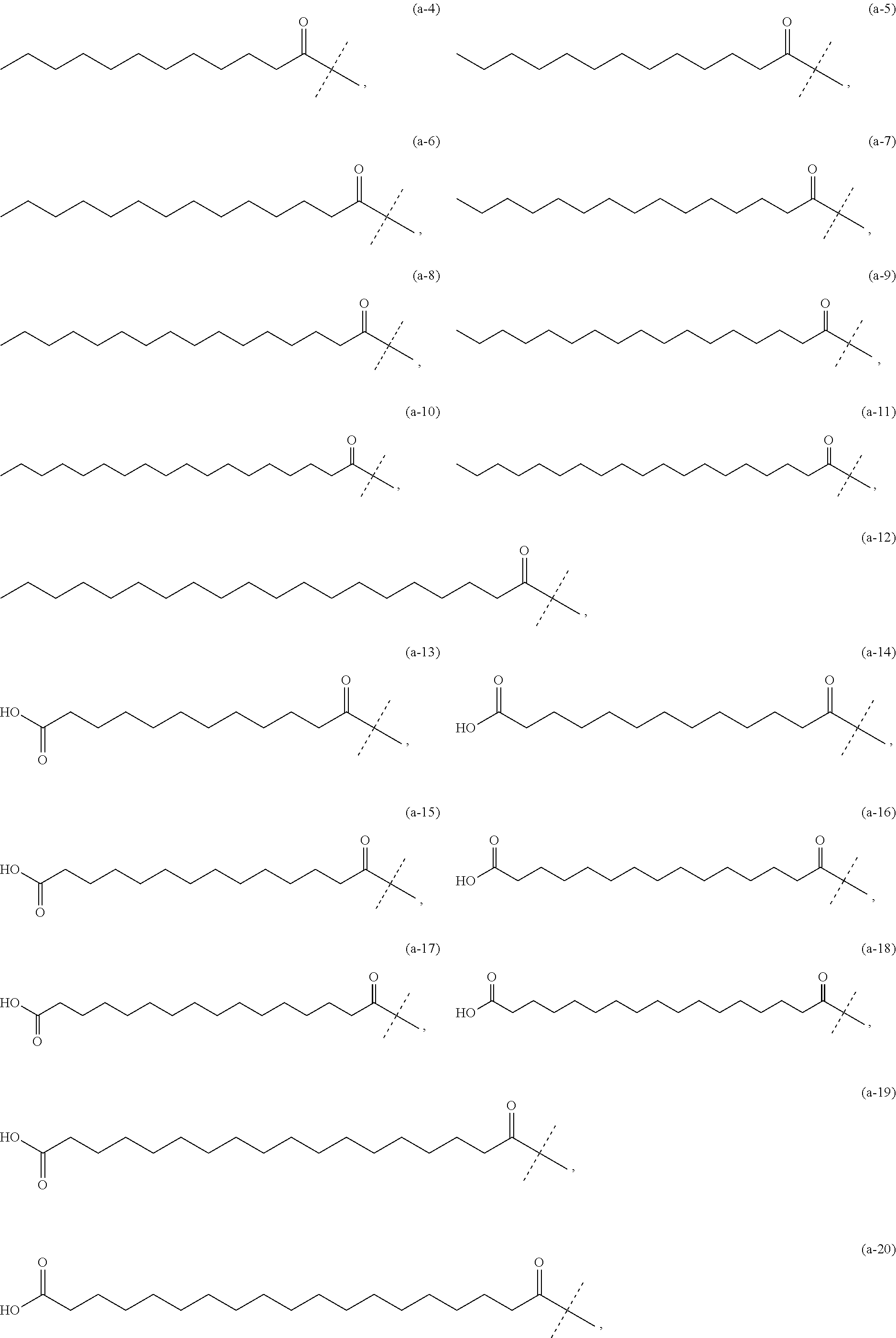

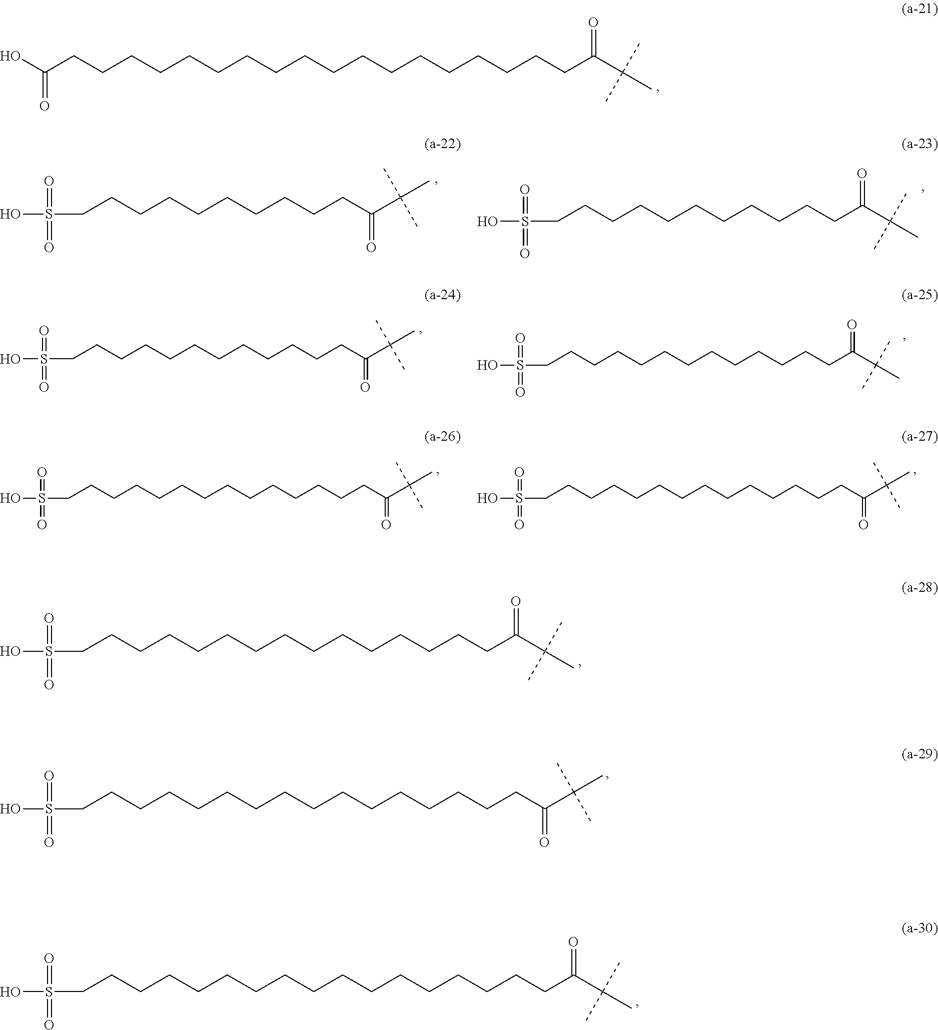

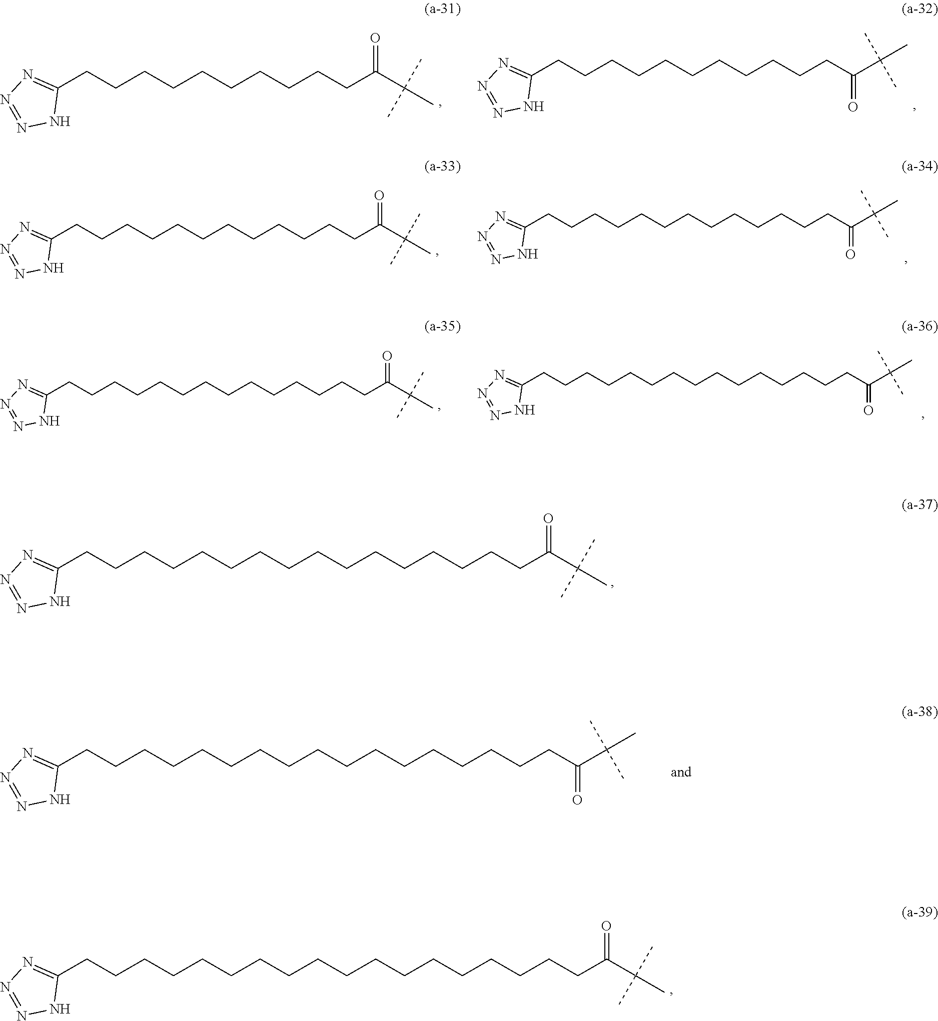

wherein the dashed lines indicate attachment to -L^{A}-.

In certain embodiments, —F⁰ is of formula (a-4). In certain embodiments, —F⁰ is of formula (a-5). In certain embodiments, —F⁰ is of formula (a-6). In certain embodiments, —F⁰ is of formula (a-7). In certain embodiments, —F⁰ is of formula (a-8). In certain embodiments, —F⁰ is of formula (a-9). In certain embodiments, —F⁰ is of formula (a-10). In certain embodiments, —F⁰ is of formula (a-11). In certain embodiments, —F⁰ is of formula (a-12). In certain embodiments, —F⁰ is of formula (a-13). In certain embodiments, —F⁰ is of formula (a-14). In certain embodiments, —F⁰ is of formula (a-15). In certain embodiments, —F⁰ is of formula (a-16). In certain embodiments, —F⁰ is of formula (a-17). In certain embodiments, —F⁰ is of formula (a-18). In certain embodiments, —F⁰ is of formula (a-19). In certain embodiments, —F⁰ is of formula (a-20). In certain embodiments, —F⁰ is of formula (a-21). In certain embodiments, —F⁰ is of formula (a-22). In certain embodiments, —F⁰ is of formula (a-23). In certain embodiments, —F⁰ is of formula (a-24). In certain embodiments, —F⁰ is of formula (a-25). In certain embodiments, —F⁰ is of formula (a-26). In certain embodiments, —F⁰ is of formula (a-27). In certain embodiments, —F⁰ is of formula (a-28). In certain embodiments, —F⁰ is of formula (a-29). In certain embodiments, —F⁰ is of formula (a-30). In certain embodiments, —F⁰ is of formula (a-31). In certain embodiments, —F⁰ is of formula (a-32). In certain embodiments, —F⁰ is of formula (a-33). In certain embodiments, —F⁰ is of formula (a-34). In certain embodiments, —F⁰ is of formula (a-35). In certain embodiments, —F⁰ is of formula (a-36). In certain embodiments, —F⁰ is of formula (a-37). In certain embodiments, —F⁰ is of formula (a-38). In certain embodiments, —F⁰ is of formula (a-39).

In certain embodiments, —F⁰ is of formula (a-4) and both -L^{A}- and -L^{B}- are absent. In certain embodiments, —F⁰ is of formula (a-5) and both -L^{A}- and -L^{B}- are absent. In certain embodiments, —F⁰ is of formula (a-6) and both -L^{A}- and -L^{B}- are absent. In certain embodiments, —F⁰ is of formula (a-7) and both -L^{A}- and -L^{B}- are absent. In certain embodiments, —F⁰ is of formula (a-8) and both -L^{A}- and -L^{B}- are absent. In certain embodiments, —F⁰ is of formula (a-9) and both -L^{A}- and -L^{B}- are absent. In certain embodiments, —F⁰ is of formula (a-10) and both -L^{A}- and -L^{B}- are absent. In certain embodiments, —F⁰ is of formula (a-11) and both -L^{A}- and -L^{B}- are absent. In certain embodiments, —F⁰ is of formula (a-12) and both -L^{A}- and -L^{B}- are absent. In certain embodiments, —F⁰ is of formula (a-13) and both -L^{A}- and -L^{B}- are absent. In certain embodiments, —F⁰ is of formula (a-14) and both -L^{A}- and -L^{B}- are absent. In certain embodiments, —F⁰ is of formula (a-15) and both -L^{A}- and -L^{B}- are absent. In certain embodiments, —F⁰ is of formula (a-16) and both -L^{A}- and -L^{B}- are absent. In certain embodiments, —F⁰ is of formula (a-17) and both -L^{A}- and -L^{B}- are absent. In certain embodiments, —F⁰ is of formula (a-18) and both -L^{A}- and -L^{B}- are absent. In certain embodiments, —F⁰ is of formula (a-19) and both -L^{A}- and -L^{B}- are absent. In certain embodiments, —F⁰ is of formula (a-20) and both -L^{A}- and -L^{B}- are absent. In certain embodiments, —F⁰ is of formula (a-21) and both -L^{A}- and -L^{B}- are absent. In certain embodiments, —F⁰ is of formula (a-22) and both -L^{A}- and -L^{B}- are absent. In certain embodiments, —F⁰ is of formula (a-23) and both -L^{A}- and -L^{B}- are absent. In certain embodiments, —F⁰ is of formula (a-24) and both -L^{A}- and -L^{B}- are absent. In certain embodiments, —F⁰ is of formula (a-25) and both -L^{A}- and -L^{B}- are absent. In certain embodiments, —F⁰ is of formula (a-26) and both -L^{A}- and -L^{B}- are absent. In certain embodiments, —F⁰ is of formula (a-27) and both -L^{A}- and -L^{B}- are absent. In certain embodiments, —F⁰ is of formula (a-28) and both -L^{A}- and -L^{B}- are absent. In certain embodiments, —F⁰ is of formula (a-29) and both -L^{A}- and -L^{B}- are absent. In certain embodiments, —F⁰ is of formula (a-30) and both -L^{A}- and -L^{B}- are absent. In certain embodiments, —F⁰ is of formula (a-31) and both -L^{A}- and -L^{B}- are absent. In certain embodiments, —F⁰ is of formula (a-32) and both -L^{A}- and -L^{B}- are absent. In certain embodiments, —F⁰ is of formula (a-33) and both -L^{A}- and -L^{B}- are absent. In certain embodiments, —F⁰ is of formula (a-34) and both -L^{A}- and -L^{B}- are absent. In certain embodiments, —F⁰ is of formula (a-35) and both -L^{A}- and -L^{B}- are absent. In certain embodiments, —F⁰ is of formula (a-36) and both -L^{A}- and -L^{B}- are absent. In certain embodiments, —F⁰ is of formula (a-37) and both -L^{A}- and -L^{B}- are absent. In certain embodiments, —F⁰ is of formula (a-38) and both -L^{A}- and -L^{B}- are absent. In Certain embodiments, —F⁰ is of formula (a-39) and both -L^{A}- and -L^{B}- are absent.

In certain embodiments, -L^{A}- is selected from the group consisting of

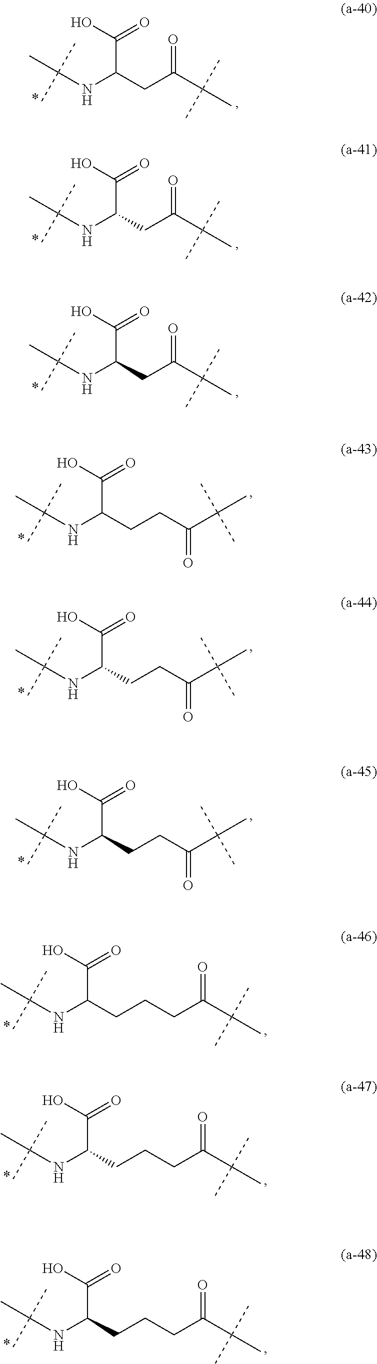

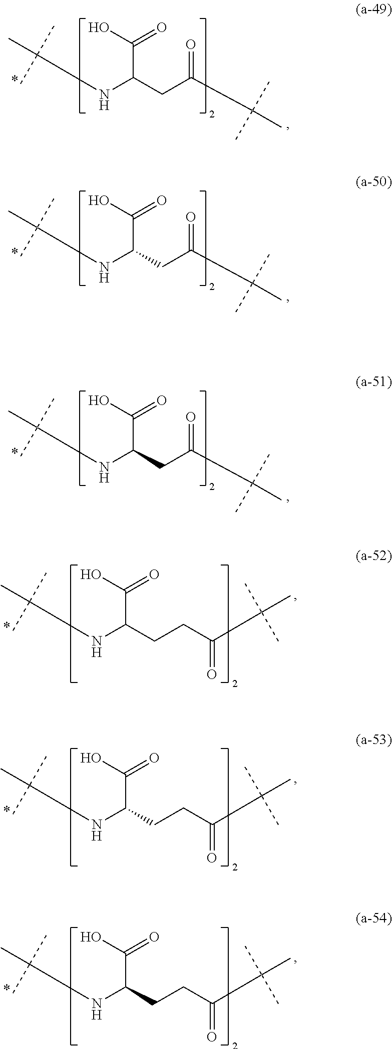

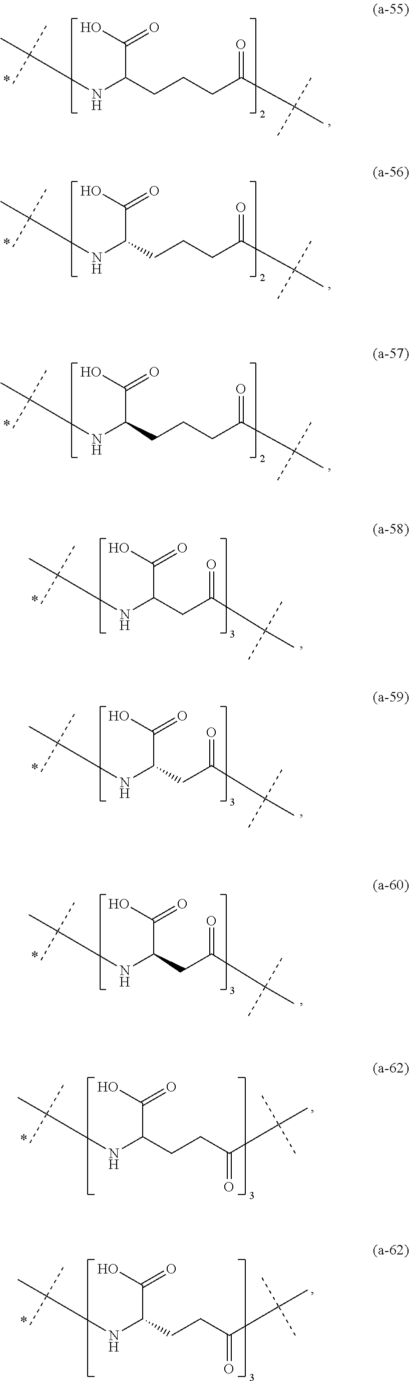

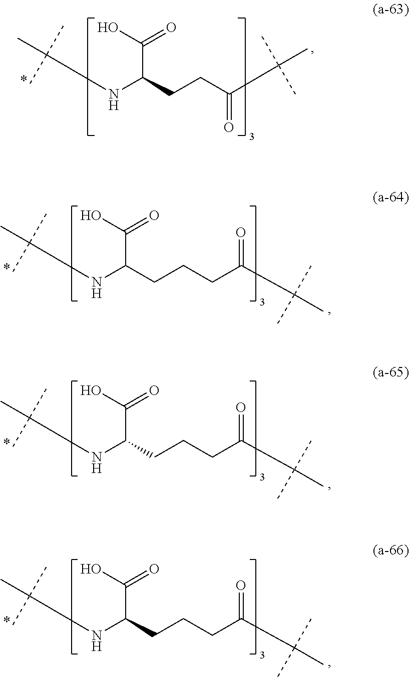

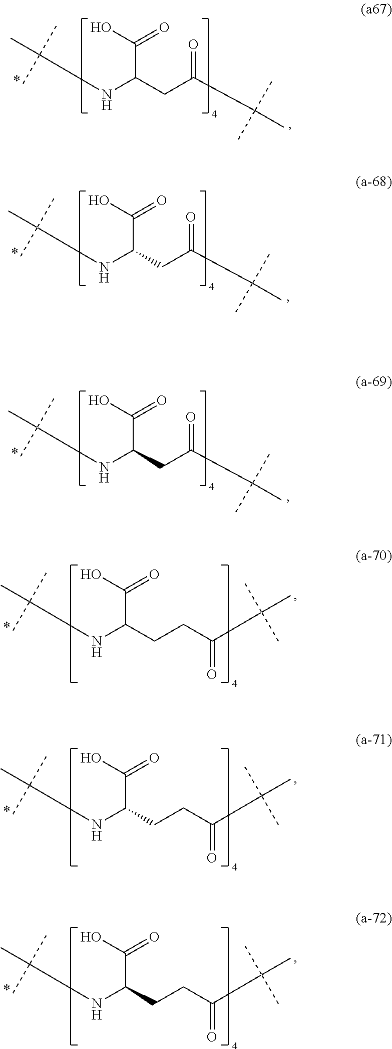

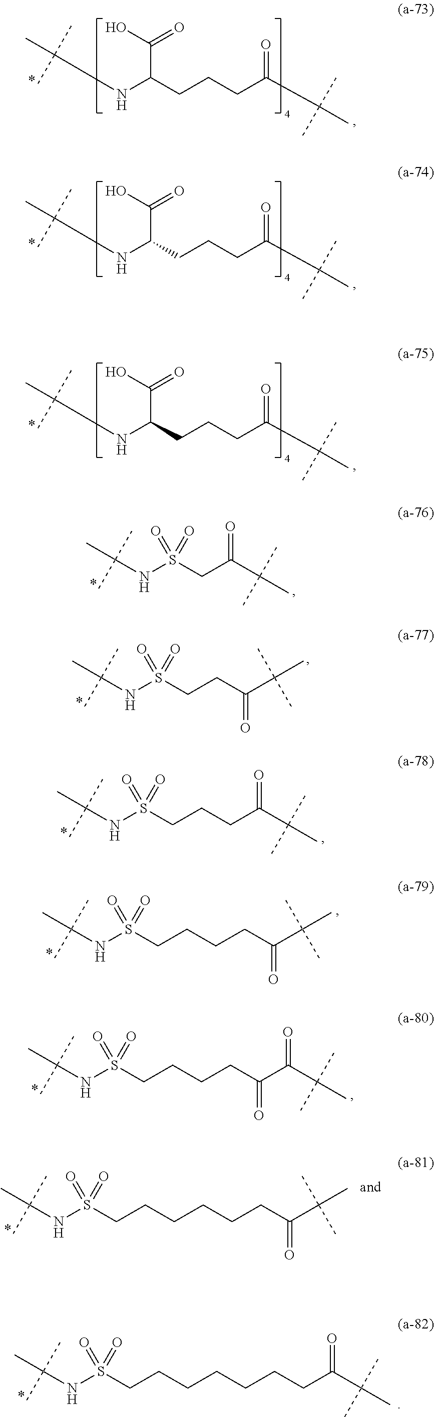

wherein the dashed lines marked with an asterisk indicate the attachment to —F⁰— and the unmarked dashed lines indicate the attachment to -L_{B}-.

In certain embodiments, L^{AA} is of formula (a-40). In certain embodiments, -L^{A}- is of formula (a-41). In certain embodiments, -L^{A}- is of formula (a-42). In certain embodiments, -L^{A}- is of formula (a-43). In certain embodiments, -L^{A}- is of formula (a-44). In certain embodiments, -L^{A}- is of formula (a-45). In certain embodiments, -L^{A}- is of formula (a-46). In certain embodiments, -L^{A}- is of formula (a-47). In certain embodiments, -L^{A}- is of formula (a-48). In certain embodiments, -L^{A}- is of formula (a-49). In certain embodiments, -L^{A}- is of formula (a-50). In certain embodiments, -L^{A}- is of formula (a-51). In certain embodiments, -L^{A}- is of formula (a-52). In certain embodiments, -L^{A}- is of formula (a-53). In certain embodiments -L^{A}- is of formula (a-54). In certain embodiments, -L^{A}- is of formula (a-55). In certain embodiments, -L^{A}- is of formula (a-56). In certain embodiments, -L^{A}- is of formula (a-57). In certain embodiments, -L^{A}- is of formula (a-58). In certain embodiments, -L^{A}- is of formula (a-59). In certain embodiments, -L^{A}- is of formula (a-60). In certain embodiments, -L^{A}- is of formula (a-61). In certain embodiments, -L^{A}- is of formula (a-62). In certain embodiments, -L^{A}- is of formula (a-63). In certain embodiments, -L^{A}- is of formula (a-64). In certain embodiments, -L^{A}- is of formula (a-65). In certain embodiments, -L^{A}- is of formula (a-66). In certain embodiments, -L^{A}- is of formula (a-67). In certain embodiments, -L^{A}- is of formula (a-68). In certain embodiments, -L^{A}- is of formula (a-69). In certain embodiments, -L^{A}- is of formula (a-70). In certain embodiments, -L^{A}- is of formula (a-71). In certain embodiments, -L^{A}- is of formula (a-72). In certain embodiments, -L^{A}- is of formula (a-73). In certain embodiments, -L^{A}- is of formula (a-74). In certain embodiments, -L^{A}- is of formula (a-75). In certain embodiments, -L^{A}- is of formula (a-76). In certain embodiments, -L^{A}- is of formula (a-77). In certain embodiments, -L^{A}- is of formula (a-78). In certain embodiments, -L^{A}- is of formula (a-79). In certain embodiments, -L^{A}- is of formula (a-80). In certain embodiments, -L^{A}- is of formula (a-81). In certain embodiments, -L^{A}- is of formula (a-82).

In certain embodiments, -L^{B}- is selected from the group consisting of

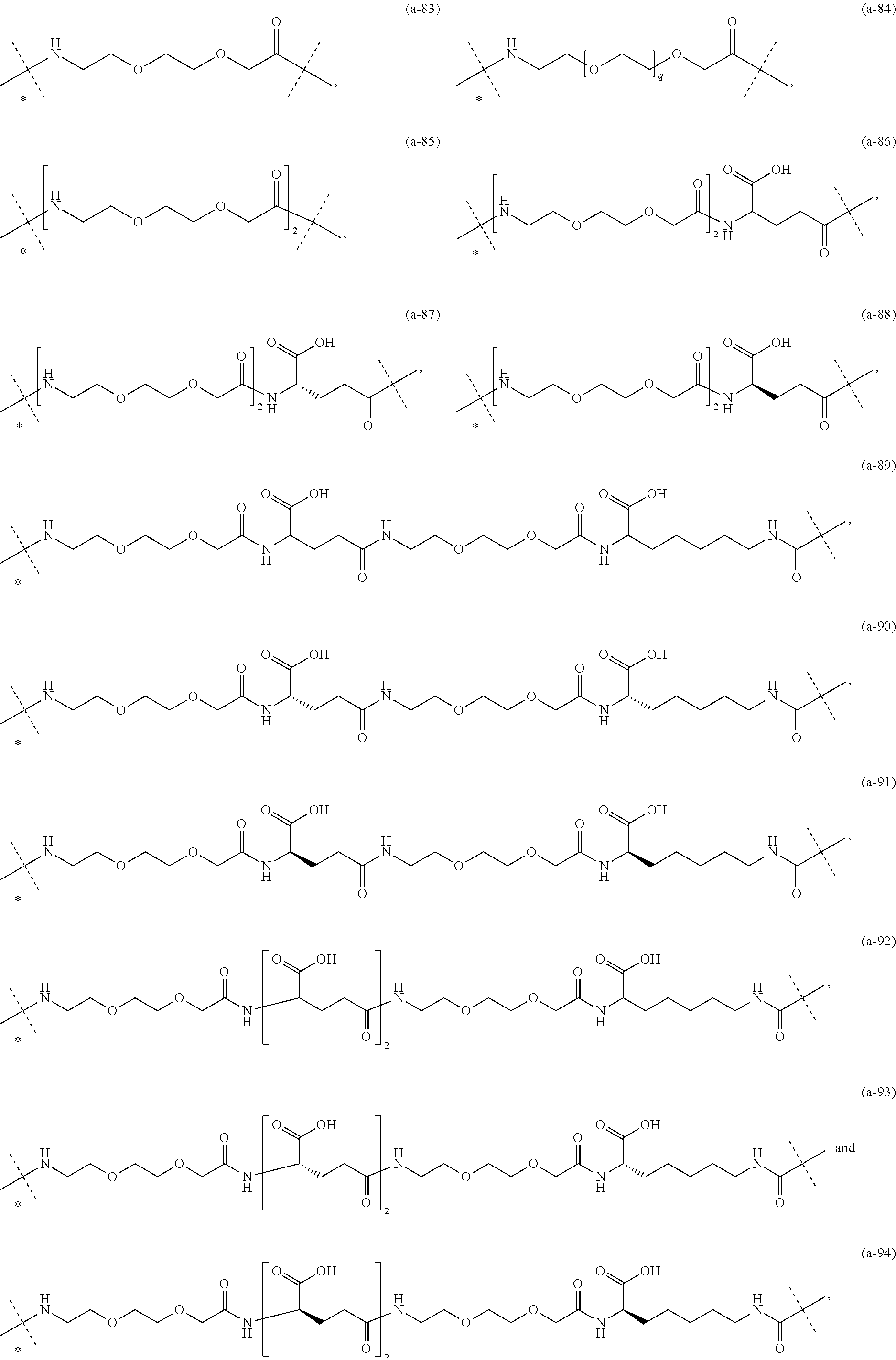

wherein the dashed line marked with the asterisk indicates attachment to -L^{A}-;
the unmarked dashed line indicates attachment to -D′-; and
q is an integer ranging from and including 2 to 50.

In certain embodiments, q is an integer ranging from and including 3 to 45. In certain embodiments, q is an integer ranging from and including 4 to 40. In certain embodiments, q is an integer ranging from and including 5 to 35. In certain embodiments, q is an integer ranging from and including 6 to 30. In certain embodiments, q is an integer ranging from and including 7 to 25. In certain embodiments, q is an integer ranging from and including 10 to 20. In certain embodiments, q is 23.

In certain embodiments, -L^{B}- is of formula (a-83). In certain embodiments, -L^{B}- is of formula (a-84). In certain embodiments, -L^{B}- is of formula (a-84) with q=23. In certain embodiments, -L^{B}- is of formula (a-85). In certain embodiments, -L^{B}- is of formula (a-86). In certain embodiments, -L^{B}- is of formula (a-87). In certain embodiments, -L^{B}- is of formula (a-88). In certain embodiments, -L^{B}- is of formula (a-89). In certain embodiments, -L^{B}- is of formula (a-90). In certain embodiments, -L^{B}- is of formula (a-91). In certain embodiments, -L^{B}- is of formula (a-92). In certain embodiments, -L^{B}- is of formula (a-93). In certain embodiments, -L^{B}- is of formula (a-94).

In certain embodiments, -AB is of formula (i):

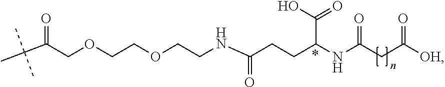

wherein the dashed line indicates attachment to -D′-; n is an integer ranging from and including 14 to 22; and
the stereocenter marked with the asterisk is either in S- or R-configuration.

In certain embodiments, -AB is of formula (i) and n is 14. In certain embodiments, -AB is of formula (i) and n is 15. In certain embodiments, -AB is of formula (i) and n is 16. In certain embodiments, -AB is of formula (i) and n is 17. In certain embodiments, -AB is of formula (i) and n is 18. In certain embodiments, -AB is of formula (i) and n is 19. In certain embodiments, -AB is of formula (i) and n is 20. In certain embodiments, -AB is of formula (i) and n is 21. In certain embodiments, -AB is of formula (i) and n is 22.

In certain embodiments, -AB is of formula (i) and n is 14 and the stereocenter marked with the asterisk is in R-configuration. In certain embodiments, -AB is of formula (i) and n is 15 and the stereocenter marked with the asterisk is in R-configuration. In certain embodiments, -AB is of formula (i) and n is 16 and the stereocenter marked with the asterisk is in R-configuration.

In certain embodiments, -AB is of formula (i) and n is 17 and the stereocenter marked with the asterisk is in R-configuration. In certain embodiments, -AB is of formula (i) and n is 18 and the stereocenter marked with the asterisk is in R-configuration. In certain embodiments, -AB is of formula (i) and n is 19 and the stereocenter marked with the asterisk is in R-configuration.

In certain embodiments, -AB is of formula (i) and n is 20 and the stereocenter marked with the asterisk is in R-configuration. In certain embodiments, -AB is of formula (i) and n is 21 and the stereocenter marked with the asterisk is in R-configuration. In certain embodiments, -AB is of formula (i) and n is 22 and the stereocenter marked with the asterisk is in R-configuration.

In certain embodiments, -AB is of formula (i) and n is 14 and the stereocenter marked with the asterisk is in S-configuration. In certain embodiments, -AB is of formula (i) and n is 15 and the stereocenter marked with the asterisk is in S-configuration. In certain embodiments, -AB is of formula (i) and n is 16 and the stereocenter marked with the asterisk is in S-configuration. In certain embodiments, -AB is of formula (i) and n is 17 and the stereocenter marked with the asterisk is in S-configuration. In certain embodiments, -AB is of formula (i) and n is 18 and the stereocenter marked with the asterisk is in S-configuration. In certain embodiments, -AB is of formula (i) and n is 19 and the stereocenter marked with the asterisk is in S-configuration. In certain embodiments, -AB is of formula (i) and n is 20 and the stereocenter marked with the asterisk is in S-configuration. In certain embodiments, -AB is of formula (i) and n is 21 and the stereocenter marked with the asterisk is in S-configuration. In certain embodiments, -AB is of formula (i) and n is 22 and the stereocenter marked with the asterisk is in S-configuration.

In certain embodiments, -AB is of formula (i-a):

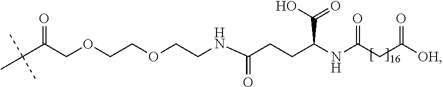

wherein the dashed line indicates attachment to -D′-.

In certain embodiments, -AB is of formula (i-b):

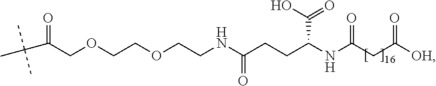

wherein the dashed line indicates attachment to -D′-.

In certain embodiments, -AB is of formula (i-c):

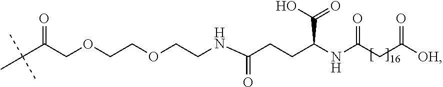

wherein the dashed line indicates attachment to -D′-.

In certain embodiments, -AB is of formula (i-d):

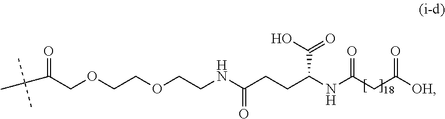

wherein the dashed line indicates attachment to -D′-.

In certain embodiments, -AB is of formula (i-e):

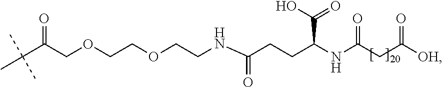

wherein the dashed line indicates attachment to -D′-.

In certain embodiments, -AB is of formula (i-f):

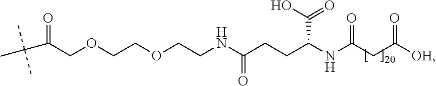

wherein the dashed line indicates attachment to -D′-.

In certain embodiments, -AB is of formula (ii)

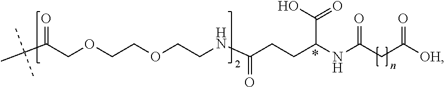

wherein the dashed line indicates attachment to -D′-;
n is an integer ranging from 14 to 22; and
the stereocenter marked with the asterisk is either in S- or R-configuration.

In certain embodiments, -AB is of formula (ii) and n is 14. In certain embodiments, -AB is of formula (ii) and n is 15. In certain embodiments, -AB is of formula (ii) and n is 16. In certain embodiments, -AB is of formula (ii) and n is 17. In certain embodiments, -AB is of formula (ii) and n is 18. In certain embodiments, -AB is of formula (ii) and n is 19. In certain embodiments, -AB is of formula (ii) and n is 20. In certain embodiments, -AB is of formula (ii) and n is 21. In certain embodiments, -AB is of formula (ii) and n is 22.

In certain embodiments, -AB is of formula (ii) and n is 14 and the stereocenter marked with the asterisk is in R-configuration. In certain embodiments, -AB is of formula (ii) and n is 15 and the stereocenter marked with the asterisk is in R-configuration. In certain embodiments, -AB is of formula (ii) and n is 16 and the stereocenter marked with the asterisk is in R-configuration. In certain embodiments, -AB is of formula (ii) and n is 17 and the stereocenter marked with the asterisk is in R-configuration. In certain embodiments, -AB is of formula (ii) and n is 18 and the stereocenter marked with the asterisk is in R-configuration. In certain embodiments, -AB is of formula (ii) and n is 19 and the stereocenter marked with the asterisk is in R-configuration. In certain embodiments, -AB is of formula (ii) and n is 20 and the stereocenter marked with the asterisk is in R-configuration. In certain embodiments, -AB is of formula (ii) and n is 21 and the stereocenter marked with the asterisk is in R-configuration. In certain embodiments, -AB is of formula (ii) and n is 22 and the stereocenter marked with the asterisk is in R-configuration.

In certain embodiments -AB is of formula (ii) and n is 14 and the stereocenter marked with the asterisk is in S-configuration. In certain embodiments -AB is of formula (ii) and n is 15 and the stereocenter marked with the asterisk is in S-configuration. In certain embodiments -AB is of formula (ii) and n is 16 and the stereocenter marked with the asterisk is in S-configuration. In certain embodiments -AB is of formula (ii) and n is 17 and the stereocenter marked with the asterisk is in S-configuration. In certain embodiments, -AB is of formula (ii) and n is 18 and the stereocenter marked with the asterisk is in S-configuration. In certain embodiments, -AB is of formula (ii) and n is 19 and the stereocenter marked with the asterisk is in S-configuration. In certain embodiments, -AB is of formula (ii) and n is 20 and the stereocenter marked with the asterisk is in S-configuration. In certain embodiments, -AB is of formula (ii) and n is 21 and the stereocenter marked with the asterisk is in S-configuration. In certain embodiments, -AB is of formula (ii) and n is 22 and the stereocenter marked with the asterisk is in S-configuration.

In certain embodiments, -AB is of formula (ii-a):

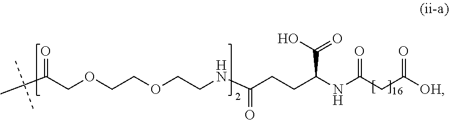

wherein the dashed line indicates attachment to -D′-.

In certain embodiments, -AB is of formula (ii-b):

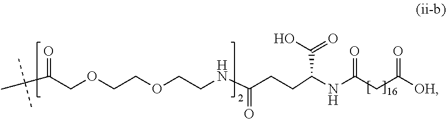

wherein the dashed line indicates attachment to -D′-.

In certain embodiments, -AB is of formula (ii-c):

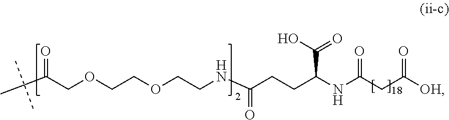

wherein the dashed line indicates attachment to -D′-.

In certain embodiments, -AB is of formula (ii-d):

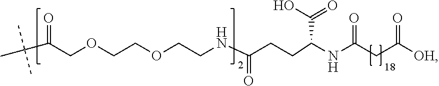

wherein the dashed line indicates attachment to -D′-.

In certain embodiments, -AB is of formula (ii-e):

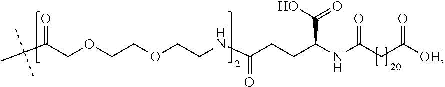

wherein the dashed line indicates attachment to -D′-.

In certain embodiments, -AB is of formula (ii-f):

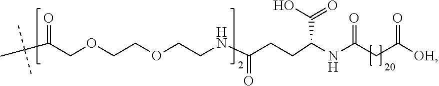

wherein the dashed line indicates attachment to -D′-.

In certain embodiments -AB is of formula (iii):

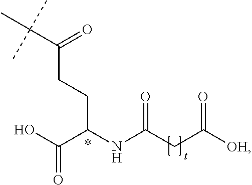

wherein
the dashed line indicates attachment to -D′-;
t is an integer ranging from and including 14 to 22; and
the stereocenter marked with the asterisk is either in S- or R-configuration.

In certain embodiments, -AB is of formula (iii) and t is 14. In certain embodiments, -AB is of formula (iii) and t is 15. In certain embodiments, -AB is of formula (iii) and t is 16. In certain embodiments, -AB is of formula (iii) and t is 17. In certain embodiments -AB is of formula (iii) and t is 18. In certain embodiments, -AB is of formula (iii) and t is 19. In certain embodiments, -AB is of formula (iii) and t is 20. In certain embodiments, -AB is of formula (iii) and t is 21. In certain embodiments, -AB is of formula (iii) and t is 22.

In certain embodiments, -AB is of formula (iii) and t is 14 and the stereocenter marked with the asterisk is in R-configuration. In certain embodiments, -AB is of formula (iii) and t is 15 and the stereocenter marked with the asterisk is in R-configuration. In certain embodiments, -AB is of formula (iii) and t is 16 and the stereocenter marked with the asterisk is in R-configuration. In certain embodiments, -AB is of formula (iii) and t is 17 and the stereocenter marked with the asterisk is in R-configuration. In certain embodiments, -AB is of formula (iii) and t is 18 and the stereocenter marked with the asterisk is in R-configuration. In certain embodiments, -AB is of formula (iii) and t is 19 and the stereocenter marked with the asterisk is in R-configuration. In certain embodiments, -AB is of formula (iii) and t is 20 and the stereocenter marked with the asterisk is in R-configuration. In certain embodiments, -AB is of formula (iii) and t is 21 and the stereocenter marked with the asterisk is in R-configuration.

In certain embodiments, -AB is of formula (iii) and t is 22 and the stereocenter marked with the asterisk is in R-configuration.

In certain embodiments, -AB is of formula (iii) and t is 14 and the stereocenter marked with the asterisk is in S-configuration. In certain embodiments -AB is of formula (iii) and t is 15 and the stereocenter marked with the asterisk is in S-configuration. In certain embodiments -AB is of formula (iii) and t is 16 and the stereocenter marked with the asterisk is in S-configuration.

In certain embodiments -AB is of formula (iii) and t is 17 and the stereocenter marked with the asterisk is in S-configuration. In certain embodiments -AB is of formula (iii) and t is 18 and the stereocenter marked with the asterisk is in S-configuration. In certain embodiments -AB is of formula (iii) and t is 19 and the stereocenter marked with the asterisk is in S-configuration. In certain embodiments -AB is of formula (iii) and t is 20 and the stereocenter marked with the asterisk is in S-configuration. In certain embodiments -AB is of formula (iii) and t is 21 and the stereocenter marked with the asterisk is in S-configuration. In certain embodiments -AB is of formula (iii) and t is 22 and the stereocenter marked with the asterisk is in S-configuration.

In certain embodiments -AB is of formula (iii-a):

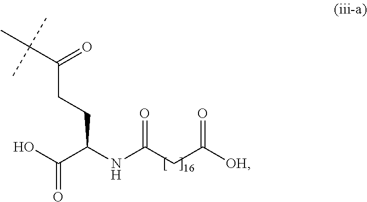

wherein the dashed line indicates attachment to -D′-.

In certain embodiments -AB is of formula (iii-b):

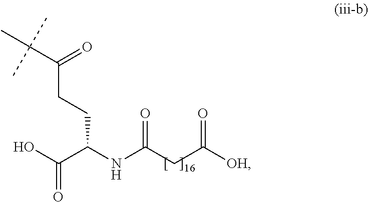

wherein the dashed line indicates attachment to -D′-.

In certain embodiments, -AB is of formula (iii-c):

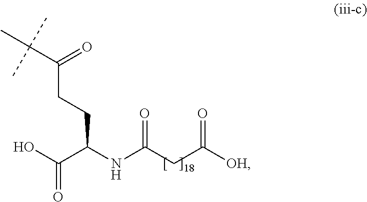

wherein the dashed line indicates attachment to -D′-.

In certain embodiments, -AB is of formula (iii-d):

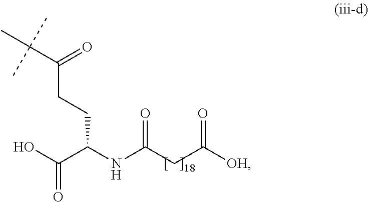

wherein the dashed line indicates attachment to -D′-.

In certain embodiments, -AB is of formula (iii-e):

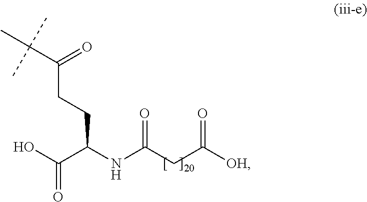

wherein the dashed line indicates attachment to -D′-.

In certain embodiments, -AB is of formula (iii-f):

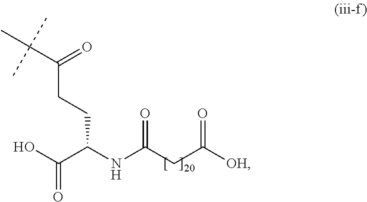

wherein the dashed line indicates attachment to -D′-.

In certain embodiments, -AB is of formula (iv):

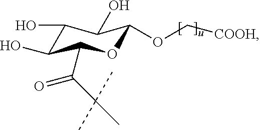

wherein the dashed line indicates attachment to -D′-, and
u is an integer ranging from and including 14 to 22.

In certain embodiments, -AB is of formula (iv) and u is 14. In certain embodiments, -AB is of formula (iv) and u is 15. In certain embodiments, -AB is of formula (iv) and u is 16. In certain embodiments, -AB is of formula (iv) and u is 17. In certain embodiments, -AB is of formula (iv) and u is 18. In certain embodiments, -AB is of formula (iv) and u is 19. In certain embodiments, -AB is of formula (iv) and u is 20. In certain embodiments, -AB is of formula (iv) and u is 21. In certain embodiments, -AB is of formula (iv) and u is 22.

In certain embodiments, -AB is of formula (v):

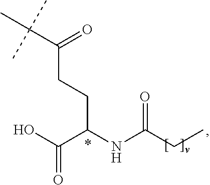

wherein
the dashed line indicates attachment to -D′-;
v is an integer ranging from and including 14 to 22; and
the stereocenter marked with the asterisk is either in S- or R-configuration.

In certain embodiments, -AB is of formula (v) and v is 14. In certain embodiments, -AB is of formula (v) and v is 15. In certain embodiments, -AB is of formula (v) and v is 16. In certain embodiments, -AB is of formula (v) and v is 17. In certain embodiments, -AB is of formula (v) and v is 18. In certain embodiments, -AB is of formula (v) and v is 19. In certain embodiments, -AB is of formula (v) and v is 20. In certain embodiments, -AB is of formula (v) and v is 21. In certain embodiments, -AB is of formula (v) and v is 22.

In certain embodiments, -AB is of formula (v) and v is 14 and the stereocenter marked with the asterisk is in R-configuration. In certain embodiments, -AB is of formula (v) and v is 15 and the stereocenter marked with the asterisk is in R-configuration. In certain embodiments, -AB is of formula (v) and v is 16 and the stereocenter marked with the asterisk is in R-configuration. In certain embodiments, -AB is of formula (v) and v is 17 and the stereocenter marked with the asterisk is in R-configuration. In certain embodiments, -AB is of formula (v) and v is 18 and the stereocenter marked with the asterisk is in R-configuration. In certain embodiments, -AB is of formula (v) and v is 19 and the stereocenter marked with the asterisk is in R-configuration.

In certain embodiments, -AB is of formula (v) and v is 20 and the stereocenter marked with the asterisk is in R-configuration. In certain embodiments, -AB is of formula (v) and v is 21 and the stereocenter marked with the asterisk is in R-configuration. In certain embodiments, -AB is of formula (v) and v is 22 and the stereocenter marked with the asterisk is in R-configuration.

In certain embodiments, -AB is of formula (v) and v is 14 and the stereocenter marked with the asterisk is in S-configuration. In certain embodiments, -AB is of formula (v) and v is 15 and the stereocenter marked with the asterisk is in S-configuration. In certain embodiments, -AB is of formula (v) and v is 16 and the stereocenter marked with the asterisk is in S-configuration. In certain embodiments, -AB is of formula (v) and v is 17 and the stereocenter marked with the asterisk is in S-configuration. In certain embodiments, -AB is of formula (v) and v is 18 and the stereocenter marked with the asterisk is in S-configuration. In certain embodiments, -AB is of formula (v) and v is 19 and the stereocenter marked with the asterisk is in S-configuration. In certain embodiments, -AB is of formula (v) and v is 20 and the stereocenter marked with the asterisk is in S-configuration. In certain embodiments, -AB is of formula (v) and v is 21 and the stereocenter marked with the asterisk is in S-configuration. In certain embodiments, -AB is of formula (v) and v is 22 and the stereocenter marked with the asterisk is in S-configuration.

In certain embodiments, -AB is of formula (v-a):

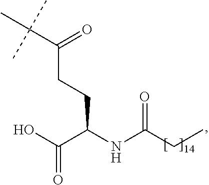

wherein the dashed line indicates attachment to -D′-.

In certain embodiments -AB is of formula (v-b):

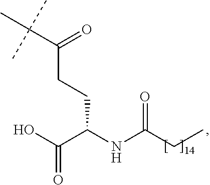

wherein the dashed line indicates attachment to -D′-.

In certain embodiments -AB is of formula (v-c):

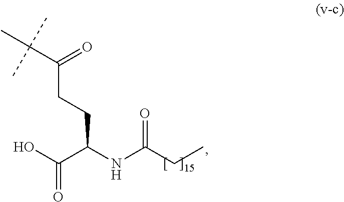

wherein the dashed line indicates attachment to -D′-.

In certain embodiments -AB is of formula (v-d):

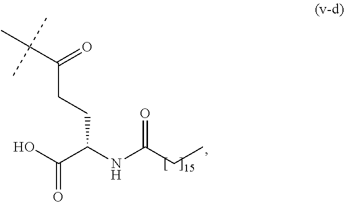

wherein the dashed line indicates attachment to -D′-.

In certain embodiments, -AB is of formula (vi-e):

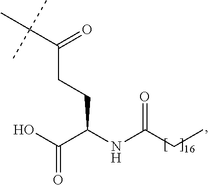

wherein the dashed line indicates attachment to -D′-.

In certain embodiments, -AB is of formula (vi-f):

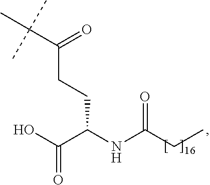

wherein the dashed line indicates attachment to -D′-.

In certain embodiments, -AB is of formula (vii):

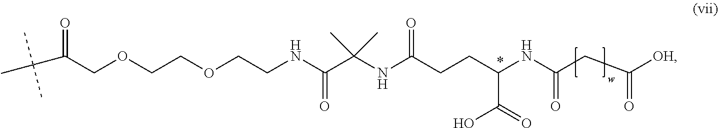

wherein the dashed line indicates attachment to -D′-;
w is an integer ranging from and including 14 to 22; and
the stereocenter marked with the asterisk is either in S- or R-configuration.

In certain embodiments, -AB is of formula (vii) and w is 14. In certain embodiments, -AB is of formula (vii) and w is 15. In certain embodiments, -AB is of formula (vii) and w is 16. In certain embodiments, -AB is of formula (vii) and w is 17. In certain embodiments, -AB is of formula (vii) and w is 18. In certain embodiments, -AB is of formula (vii) and w is 19. In certain embodiments, -AB is of formula (vii) and w is 20. In certain embodiments, -AB is of formula (vii) and w is 21. In certain embodiments, -AB is of formula (vii) and w is 22. In certain embodiments, -AB is of formula (vii) and w is 14 and the stereocenter marked with the asterisk is in R-configuration. In certain embodiments, -AB is of formula (vii) and w is 15 and the stereocenter marked with the asterisk is in R-configuration. In certain embodiments, -AB is of formula (vii) and w is 16 and the stereocenter marked with the asterisk is in R-configuration. In certain embodiments, -AB is of formula (vii) and w is 17 and the stereocenter marked with the asterisk is in R-configuration. In certain embodiments, -AB is of formula (vii) and w is 18 and the stereocenter marked with the asterisk is in R-configuration. In certain embodiments, -AB is of formula (vii) and w is 19 and the stereocenter marked with the asterisk is in R-configuration. In certain embodiments, -AB is of formula (vii) and w is 20 and the stereocenter marked with the asterisk is in R-configuration. In certain embodiments, -AB is of formula (vii) and w is 21 and the stereocenter marked with the asterisk is in R-configuration. In certain embodiments, -AB is of formula (vii) and w is 22 and the stereocenter marked with the asterisk is in R-configuration.

In certain embodiments, -AB is of formula (vii) and w is 14 and the stereocenter marked with the asterisk is in S-configuration. In certain embodiments, -AB is of formula (vii) and w is 15 and the stereocenter marked with the asterisk is in S-configuration. In certain embodiments, -AB is of formula (vii) and w is 16 and the stereocenter marked with the asterisk is in S-configuration. In certain embodiments, -AB is of formula (vii) and w is 17 and the stereocenter marked with the asterisk is in S-configuration. In certain embodiments, -AB is of formula (vii) and w is 18 and the stereocenter marked with the asterisk is in S-configuration. In certain embodiments -AB is of formula (vii) and w is 19 and the stereocenter marked with the asterisk is in S-configuration. In certain embodiments, -AB is of formula (vii) and w is 20 and the stereocenter marked with the asterisk is in S-configuration. In certain embodiments, -AB is of formula (vii) and w is 21 and the stereocenter marked with the asterisk is in S-configuration. In certain embodiments, -AB is of formula (vii) and w is 22 and the stereocenter marked with the asterisk is in S-configuration.

In certain embodiments, -AB is of formula (vii-a):

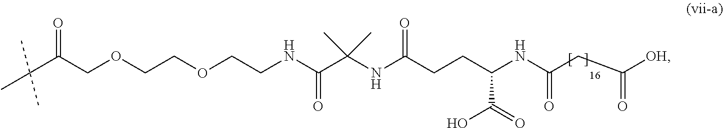

wherein the dashed line indicates attachment to -D′-.

In certain embodiments, -AB is of formula (vii-b):

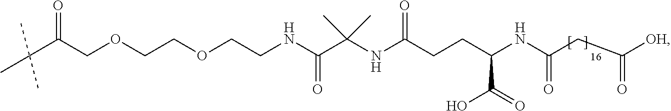

wherein the dashed line indicates attachment to -D′-.

In certain embodiments, -AB is of formula (vii-c):

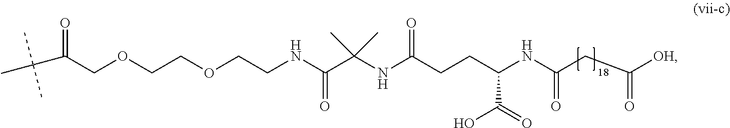

wherein the dashed line indicates attachment to -D′-.

In certain embodiments -AB is of formula (vii-d):

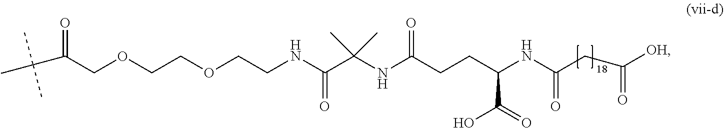

wherein the dashed line indicates attachment to -D′-.

In certain embodiments, -AB is of formula (vii-e):

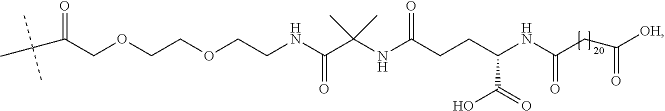

wherein the dashed line indicates attachment to -D′-.

In certain embodiments -AB is of formula (vii-f):

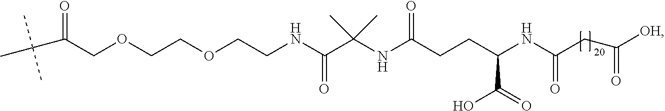

wherein the dashed line indicates attachment to -D′-.

In certain embodiments -AB is of formula (viii):

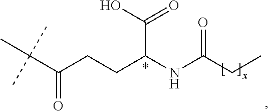

wherein the dashed line indicates attachment to -D′-;
w is an integer ranging from and including 14 to 22; and
the stereocenter marked with the asterisk is either in S- or R-configuration.

In certain embodiments, -AB is of formula (viii) and x is 14. In certain embodiments -AB is of formula (viii) and x is 15. In certain embodiments, -AB is of formula (viii) and x is 16. In certain embodiments -AB is of formula (viii) and x is 17. In certain embodiments, -AB is of formula (viii) and x is 18. In certain embodiments, -AB is of formula (viii) and x is 19. In certain embodiments, -AB is of formula (viii) and x is 20. In certain embodiments, -AB is of formula (viii) and x is 21. In certain embodiments, -AB is of formula (viii) and x is 22.

In certain embodiments, -AB is of formula (viii) and x is 14 and the stereocenter marked with the asterisk is in R-configuration. In certain embodiments, -AB is of formula (viii) and x is 15 and the stereocenter marked with the asterisk is in R-configuration. In certain embodiments, -AB is of formula (viii) and x is 16 and the stereocenter marked with the asterisk is in R-configuration. In certain embodiments, -AB is of formula (viii) and x is 17 and the stereocenter marked with the asterisk is in R-configuration. In certain embodiments, -AB is of formula (viii) and x is 18 and the stereocenter marked with the asterisk is in R-configuration. In certain embodiments, -AB is of formula (viii) and x is 19 and the stereocenter marked with the asterisk is in R-configuration. In certain embodiments, -AB is of formula (viii) and x is 20 and the stereocenter marked with the asterisk is in R-configuration. In certain embodiments, -AB is of formula (viii) and x is 21 and the stereocenter marked with the asterisk is in R-configuration. In certain embodiments, -AB is of formula (viii) and x is 22 and the stereocenter marked with the asterisk is in R-configuration.

In certain embodiments, -AB is of formula (viii) and x is 14 and the stereocenter marked with the asterisk is in S-configuration. In certain embodiments, -AB is of formula (viii) and x is 15 and the stereocenter marked with the asterisk is in S-configuration. In certain embodiments, -AB is of formula (viii) and x is 16 and the stereocenter marked with the asterisk is in S-configuration. In certain embodiments, -AB is of formula (viii) and x is 17 and the stereocenter marked with the asterisk is in S-configuration. In certain embodiments, -AB is of formula (viii) and x is 18 and the stereocenter marked with the asterisk is in S-configuration. In certain embodiments, -AB is of formula (viii) and x is 19 and the stereocenter marked with the asterisk is in S-configuration. In certain embodiments, -AB is of formula (viii) and x is 20 and the stereocenter marked with the asterisk is in S-configuration. In certain embodiments, -AB is of formula (viii) and x is 21 and the stereocenter marked with the asterisk is in S-configuration. In certain embodiments, -AB is of formula (viii) and x is 22 and the stereocenter marked with the asterisk is in S-configuration.

In certain embodiments, -AB is of formula (viii-a):

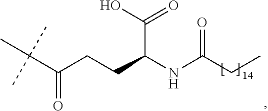

wherein the dashed line indicates attachment to -D′-.

In certain embodiments, —At is of formula viii-b):

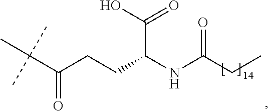

wherein the dashed line indicates attachment to -D′-.

In certain embodiments, -AB is of formula (viii-c):

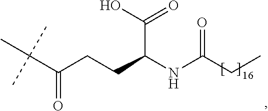

wherein the dashed line indicates attachment to -D′-.

In certain embodiments, -AB is of formula (viii-d):

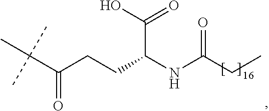

wherein the dashed line indicates attachment to -D′-.

In certain embodiments, -AB is of formula (viii-e):

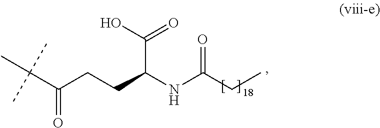

wherein the dashed line indicates attachment to -D′-.

In certain embodiments, -AB is of formula (viii-f):

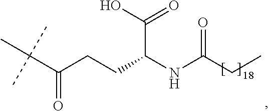

wherein the dashed line indicates attachment to -D′-.

In certain embodiments, -AB is a peptidic albumin-binding moiety.

If -D′- is a peptide or protein drug moiety, such peptidic moiety -AB may be fused to the N- or C-terminus of -D′-, either directly or with a peptidic spacer between -D′- and -AB.

In certain embodiments, -AB is selected from the group consisting of:

(SEQ ID NO: 34)
WWEQDRDWDFDVFGGGTP;
(SEQ ID NO: 35)
DICLPRWGCLW,

wherein the cysteines at position 3 and 9 are connected via a disulfide bridge;

(SEQ ID NO: 36)
RLIEDICLPRWGCLWEDD,

wherein the cysteines at position 7 and 13 are connected via a disulfide bridge;

(SEQ ID NO: 37)
LAEAKVLANRELDKYGVSDFYKRLINKAKTVEGVEALKLHILAALP;

(SEQ ID NO: 38)
IAEAKEAANAELDSYGVSDFYKRLIDKAKTVEGVEALKDAILAALP;
and
(SEQ ID NO: 39)
X₁EYEX₂EYE,

wherein X₁ is fluorescein-(AEEA), X₂ is K(palmitate), and AEEA is 2-(2-(2-aminoethoxy)acetyl.

In certain embodiments, -AB is of SEQ ID NO:34. In certain embodiments, -AB is of SEQ ID NO:35. In certain embodiments, -AB is of SEQ ID NO:36. In certain embodiments, -AB is of SEQ ID NO:37. In certain embodiments, -AB is of SEQ ID NO:38. In certain embodiments, -AB is of SEQ ID NO:39.

In certain embodiments, -D′- is a protein or peptide drug moiety and -AB is conjugated to -D′- to a functional group of -D′- provided by the N-terminal amine, the C-terminal carboxyl or a side chain of an amino acid residue. In certain embodiments, -AB is conjugated to the N-terminal amine functional group of -D′-. In certain embodiments, -AB is conjugated to the C-terminal carboxyl functional group. In certain embodiments, -AB is conjugated to a functional group provided by an amino acid residue, such as by a lysine, serine, aspartic acid, glutamic acid, arginine, histidine, threonine, glutamine, asparagine, cysteine, proline, tyrosine or tryptophan. In certain embodiments, -AB is conjugated to the functional group of the side chain of a lysine. In certain embodiments, -AB is conjugated to the functional group of the side chain of a serine. In certain embodiments, -AB is conjugated to the functional group of the side chain of an aspartic acid. In certain embodiments, -AB is conjugated to the functional group of the side chain of a glutamic acid. In certain embodiments, -AB is conjugated to the functional group of the side chain of an arginine. In certain embodiments, -AB is conjugated to the functional group of the side chain of a histidine. In certain embodiments, -AB is conjugated to the functional group of the side chain of a threonine. In certain embodiments, -AB is conjugated to the functional group of the side chain of a glutamine. In certain embodiments, -AB is conjugated to the functional group of the side chain of an asparagine. In certain embodiments, -AB is conjugated to the functional group of the side chain of a cysteine. In certain embodiments, -AB is conjugated to the functional group of the side chain of a proline. In certain embodiments, -AB is conjugated to the functional group of the side chain of a tyrosine. In certain embodiments, -AB is conjugated to the functional group of the side chain of a tryptophan.

In certain embodiments, -D′-AB is selected from the group consisting of semaglutide, liraglutide, ecnoglutide, GZR18 and GL0034.

In certain embodiments -D′-AB is semaglutide. Semaglutide is a compound of formula

(SEQ ID NO: 40)
HX₁EGTFTSDVSSYLEGQAAKEFIAWLVRGRG,

wherein
X₁ is α-aminoisobutyric acid (Aib); and
the lysine at position 20 is chemically modified through conjugation to the epsilon-amine group of the lysine side-chain with

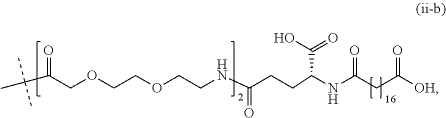

wherein
the dashed line indicates attachment to the epsilon-amine group of the lysine side chain of the lysine at position 20.

In certain embodiments, -D′-AB is liraglutide. Liraglutide is a compound of formula

(SEQ ID NO: 41)
HAEGTFTSDVSSYLEGQAAKEFIAWLVRGRG,

wherein
the lysine at position 20 is chemically modified through conjugation to the epsilon-amine group of the lysine side chain with

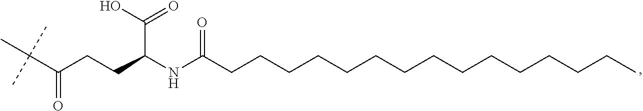

wherein
the dashed line indicates attachment to the epsilon-amine group of the lysine side chain of the lysine at position 20.

In certain embodiments -D′-AB is ecnoglutide. Ecnoglutide is a compound of formula

(SEQ ID NO: 42)
HVEGTFTSDVSSYLEEQAAREFIKWLVRGRG,

wherein
the lysine at position 24 is chemically modified through conjugation to the epsilon-amine group of the lysine side chain with

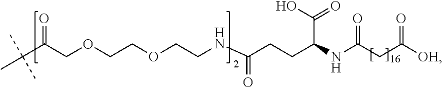

wherein the dashed line indicates attachment to the epsilon-amine group of the lysine side chain of the lysine at position 24.

In certain embodiments -D′-AB is GZR18. GZR18 is a compound of formula

(SEQ ID NO: 43)
HGEGTFTSDVSSYLEGQAAKEFIAWLVRGRG,

wherein
the lysine at position 20 is chemically modified through conjugation to the epsilon-amine group of the lysine side-chain with

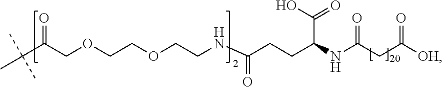

wherein the dashed line indicates attachment to the epsilon amine group of the lysine side chain of the lysine at position 20.

In certain embodiments -D′-AB is GL0034. GL0034 is a compound of formula

(SEQ ID NO: 44)
HX₁EGTFTSDVSSYLEGQAAKEFIAWLVRGRGL,

wherein
X₁ is Aib; and
the lysine at position 20 is chemically modified through conjugation to the epsilon-amine group of the lysine side-chain with

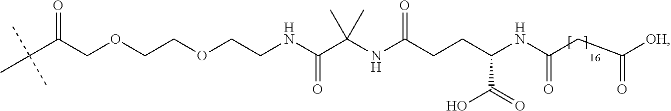

wherein the dashed line indicates attachment to the epsilon amine group of the lysine side chain of the lysine at position 20.

In certain embodiments -D′- or -D′-AB is a dual GLP-1 receptor agonist selected from the group consisting of tirzepatide, cotadutide, BI-456906, pemvidutide and mazdutide.

In certain embodiments, -D′-AB is a dual GLP-1 receptor agonist that activates the GLP-1 receptor and the GIP receptor. An example for such dual GLP-1 receptor agonist is tirzepatide.

In certain embodiments -D′-AB is tirzepatide. Tirzepatide is a compound of formula

(SEQ ID NO: 45)
YX₁EGTFTSDYSIX₂LDKIAQKAFVQWLIAGGPSSGAPPPS,

wherein
X₁ is Aib;
X₂ is Aib;
the lysine at position 20 is chemically modified through conjugation to the epsilon-amine group of the lysine side-chain with ([2-(2-amino-ethoxy)-ethoxy]-acetyl)₂-(γGlu)₁-CO—(CH₂)₁₈—CO₂H; and
the C-terminal serine, i.e. the serine at position 39, is amidated as a C-terminal primary amide.

The moiety ([2-(2-amino-ethoxy)-ethoxy]-acetyl)₂-(γGlu)₁-CO—(CH₂)₁₈—CO₂H has the following structure

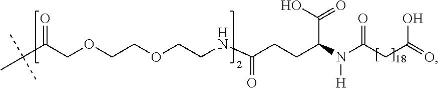

wherein the dashed line indicates attachment to -D′-.

In certain embodiments -D′-AB is a dual GLP-1 receptor agonist that activates the GLP-1 receptor and the glucagon receptor selected from the group consisting of cotadutide, BI-456906, pemvidutide and mazdutide.

In certain embodiments, -D′-AB is cotadutide. Cotadutide is a compound of formula

(SEQ ID NO: 46)
HSQGTFTSDKSEYLDSERARDFVAWLEAGG,

wherein
the lysine at position 10 is chemically modified through conjugation to the epsilon-amine group of the lysine side-chain with γ-Glu-palmitoyl.

The moiety γ-Glu-palmitoyl has the following structure:

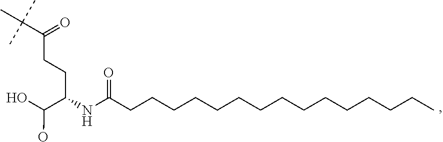

wherein the dashed line indicates attachment to -D′-.

In certain embodiments, -D′-AB is BI-456906. BI-456906 is a compound of formula

(SEQ ID NO: 47)
HX₁QGTFTSDYSKYLDERAAKDFIKWLESA,

wherein
X₁ is 1-amino-cyclobutanecarboxylic acid (Ac4c);
the lysine at position 24 is chemically modified through conjugation to the epsilon-amine group of the lysine side-chain with [17-carboxy-heptadecanoyl]-isoGlu-GSGSGG; and
the C-terminal alanine, i.e. the alanine at position 29, is amidated as a C-terminal primary amide.

The moiety [17-carboxy-heptadecanoyl]-isoGlu-GSGSGG has the following structure:

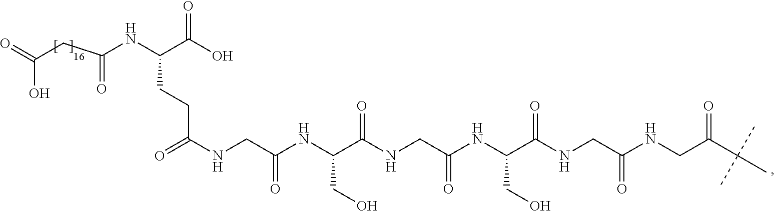

wherein the dashed line indicates attachment to -D′-.

In certain embodiments, -D′-AB is pemvidutide. Pemvidutide is a compound of formula

(SEQ ID NO: 48)
HX₁QGTFTSDYSKYLDEKAAKEFIQWLLQT,

wherein
X₁ is Aib;
the glutamic acid at position 16 and the lysine at position 20 are connected via a lactam bridge;
the lysine at position 17 is chemically modified through conjugation to the epsilon-amine group of the lysine side-chain with glucuronic acid C-18, which is a moiety of formula

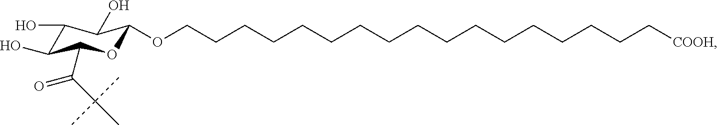

wherein
the dashed line indicates attachment to the epsilon-amine group of the lysine at position 17; and
the C-terminal threonine, i.e. the threonine at position 29, is amidated as a C-terminal primary amide

In certain embodiments, -D′-AB is mazdutide. Mazdutide is a compound of formula

(SEQ ID NO: 49)
HX₁QGTFTSDYSKYLDEKKAKEFVEWLLEGGPSSG,

wherein
X₁ is Aib,
the lysine at position 20 is chemically modified by conjugation of the epsilon-amine group of the lysine side chain with ([2-(2-amino-ethoxy)-ethoxy]-acetyl)₂-(γ-Glu)—CO—(CH₂)₁₈—CO₂H; and
the C-terminal glycine, i.e. the glycine at position 34, is amidated.

The moiety ([2-(2-amino-ethoxy)-ethoxy]-acetyl)₂-(γ-Glu)—CO—(CH₂)₁₈—CO₂H has the following structure:

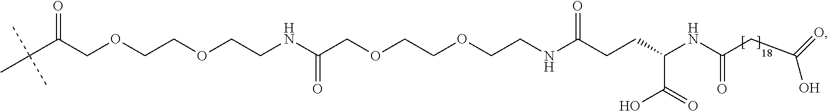

wherein the dashed line indicates attachment to -D-.

In certain embodiments, -D′-AB is a dual GLP-1 receptor agonist that activates the GLP-1 receptor and the GLP-2 receptor, such as dapiglutide. Dapiglutide is a compound of formula

(SEQ ID NO: 50)
HX₁EGSFTSELATILDKQAARDFIAWLIQHKITD,

wherein
X₁ is Aib; and
the lysine in position 16 is chemically modified by conjugation of the epsilon-amino-group of the lysine side chain with [17-carboxy-heptadecanoyl]-isoGlu.

The moiety [17-carboxy-heptadecanoyl]-isoGlu has the following structure:

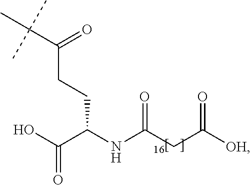

wherein the dashed line indicates attachment to -D′-.

In certain embodiments, -D′-AB is retatrutide, which is a triple GLP-1 receptor agonist that activates the GLP-1 receptor, the GIP receptor and the GCG receptor. Retatrutide is a compound of formula

(SEQ ID NO: 51)
YX₁QGTFTSDYSIX₂LDKKAQX₃AFIEYLLEGGPSSGAPPPS,

wherein
X₁ is Aib;
X₂ is α-methyl-leucine (αMeL);
X₃ is Aib;
the lysine at position 17 is chemically modified by conjugation of the epsilon-amine group of the lysine side chain with (2-[2-(2-amino-ethoxy)-ethoxy]-acetyl)-(γGlu)—CO—(CH₂)₁₈—CO₂H; and
the C-terminal serine, i.e. the serine at position 39, is amidated.

Retatrutide can also be described as Y-Aib-QGTFTSDYSI-αMeL-LDKK ((2-[2-(2-amino-ethoxy)-ethoxy]-acetyl)-(γGlu)—CO—(CH₂)₁₈—CO₂H) AQ-Aib-AFIEYLLEGGPSSGAPPPS-NH₂ (SEQ ID NO:XX).

The moiety (2-[2-(2-amino-ethoxy)-ethoxy]-acetyl)-(γGlu)—CO—(CH₂)₁₈—CO₂H has the following structure:

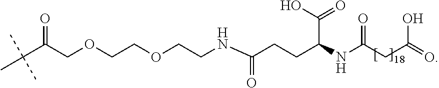

In certain embodiments -D′-AB is a compound of formula

(SEQ ID NO: 52)
k(γE-(miniPEG)₂-γE-COC₁₆H₃₂CO₂H)(N-Me)
GSVSEIQLMHNLGKHLNSMERVEW
LRKKLQDVHK(γE-(miniPEG)₂-γE-COC₁₆H₃₂CO₂H)-OH,

wherein
k is d-Lys;
γE is the l-isomer of gamma, glutamic acid;
miniPEG is COCH₂OCH₂CH₂OCH₂CH₂NH;
COC₁₆H₃₂CO₂H is C18 diacid;
(N-Me)G is sarcosine;
K is l-isomer of lysine; and
—OH designates the C-terminal amino acid has a terminal carboxylic acid.

In certain embodiments -D′-AB is a compound of formula

(SEQ ID NO: 53)
k(γE-(miniPEG)₂-γE-COC₁₆H₃₂CO₂H)(N-Me)
GSVSEIQLMHNLGKHLNSMERVEW
LRKKLQDVHK(γE-(miniPEG)₂-γE-COC₁₆H₃₂CO₂H)-OH,

wherein
k is d-Lys;
γE is the l-isomer of gamma, glutamic acid;
(miniPEG)₂ is COCH₂OCH₂CH₂OCH₂CH₂NH;
COC₁₆H₃₂CO₂H is C18 diacid;
(N-Me)G is sarcosine;
K is l-isomer of lysine; and
—OH designates the C-terminal amino acid has a terminal carboxylic acid.

In certain embodiments the distance between two moieties -AB is such that they are capable of binding to a binding domain of two different albumins or to two different binding domains of the same albumin. In certain embodiments the distance between two moieties -AB is such that they are capable of binding to a binding domain of two different albumins.

In another aspect the present invention relates to a pharmaceutical composition comprising at least one compound of the present invention or a pharmaceutically acceptable salt thereof and at least one excipient.

In another aspect the present invention relates to a compound of the present invention or a pharmaceutically acceptable salt thereof or a pharmaceutical composition of the present invention for use as a medicament, optionally in combination with one or more additional therapeutically active compounds.

The present invention also relates to a compound or its pharmaceutically acceptable salt thereof or a pharmaceutical composition of the present invention for use as a medicament.

The present invention also relates to a compound or its pharmaceutically acceptable salt thereof or a pharmaceutical composition of the present invention for use in the manufacture of a medicament.

Such medicament may be used in the treatment or prevention of a disease that can be treated or prevented with the corresponding free drug.

Another aspect of the present invention relates to a compound of the present invention or a pharmaceutically acceptable salt thereof or a pharmaceutical composition comprising said compound of the present invention for use in a method of treating or preventing a disease that can be treated or prevented with the corresponding free drug or its pharmaceutically acceptable salt thereof, optionally in combination with one or more additional therapeutically active compounds.

In another aspect the present invention relates to a method of treating a patient having a disease that can be treated or prevented with the corresponding free drug, wherein the method comprises the step of administering a pharmaceutically acceptable amount of a compound or a pharmaceutically acceptable salt thereof or a pharmaceutical composition of the present invention to a patient in need thereof, optionally in combination with one or more additional therapeutically active compounds.

Administration of a compound, a pharmaceutically acceptable salt thereof or a pharmaceutical composition of the present invention may be via external application, injection or infusion, including intraarticular, periarticular, intradermal, subcutaneous, intramuscular, intravenous, intraosseous, intraperitoneal, intrathecal, intracapsular, intraorbital, intravitreal, intratympanic, intravesical, intracardiac, transtracheal, subcuticular, subcapsular, subarachnoid, intraspinal, intraventricular, intrasternal injection and infusion; direct delivery to the brain via implanted device allowing delivery of the invention or the like to brain tissue or brain fluids (e.g., Ommaya Reservoir), direct intracerebroventricular injection or infusion, injection or infusion into brain or brain associated regions, injection into the subchoroidal space, retro-orbital injection and ocular instillation. In certain embodiments the medicament is for subcutaneous injection, which may be done with a pen injector or via a syringe.

A compound, in which the corresponding free drug is a GLP-1 receptor agonist, or its pharmaceutically acceptable salt thereof or a pharmaceutical composition comprising such compound may be used in the treatment or prevention of a disease selected from the group consisting of (i) all forms of diabetes, (ii) obesity, (iii) non-alcoholic fatty liver disease (NAFLD) and non-alcoholic steatohepatitis (NASH), (iv) cardiovascular disease, (v) neurodegenerative disorders, (vi) chronic kidney disease (CKD), (vii) diabetic kidney disease (DKD), (viii) peripheral arterial disease (PAD), and/or (ix) heart failure (HF).

Exemplary cardiovascular diseases may be selected from the group consisting of syndrome X, atherosclerosis, myocardial infarction, coronary heart disease, reperfusion injury, stroke, cerebral ischemia, an early cardiac or early cardiovascular disease, left ventricular hypertrophy, coronary artery disease, hypertension, essential hypertension, acute hypertensive emergency, cardiomyopathy, heart insufficiency, exercise intolerance, acute and/or chronic heart failure, arrhythmia, cardiac dysrhythmia, syncopy, angina pectoris, cardiac bypass and/or stent reocclusion, intermittent claudication (atheroschlerosis oblitterens), diastolic dysfunction, and/or systolic dysfunction; and reduction of blood pressure, such as reduction of systolic blood pressure.

A compound, in which the corresponding free drug is a GLP-1 receptor agonist, or its pharmaceutically acceptable salt thereof or a pharmaceutical composition of the present invention may be also used in the treatment or prevention of dyslipidemia and/or diseases where one or more of the following clinical outcomes are the treatment goal: lowering total serum lipids; increasing HDL; lowering small, dense LDL; lowering VLDL; lowering triglycerides; lowering cholesterol; lowering plasma levels of lipoprotein a (Lp(a)) in a human; inhibiting generation of apolipoprotein A (apo(A)).

Exemplary neurodegenerative disorders may be selected from the group consisting of Alzheimer's disease and Parkinson's disease.

Exemplary forms of HF may be selected from the group consisting of heart failure with reduced ejection fraction (HFrEF), heart failure with mid-range ejection fraction (HFmrEF) and heart failure with preserved ejection fraction (HFpEF).

In certain embodiments the compound or pharmaceutically acceptable salt thereof of the present invention is used for the treatment of non-alcoholic fatty liver disease (NAFLD) and non-alcoholic steatohepatitis (NASH).

A compound, in which the corresponding free drug is a GLP-1 receptor agonist, or its pharmaceutically acceptable salt thereof or a pharmaceutical composition of the present invention may be also used in the treatment of obesity and/or eating disorders, where one or more of the following clinical outcomes are the treatment goal: decreasing food intake, increasing energy expenditure, reducing body weight, suppressing appetite, inducing satiety.

A compound, in which the corresponding free drug is a GLP-1 receptor agonist, or its pharmaceutically acceptable salt thereof or a pharmaceutical composition of the present invention may also be combined with one or more additional drugs, such as drugs selected from cardiovascular agents, antidiabetic agents, and/or anti-obesity agents. Examples of these pharmacologically active substances are: inotropes, beta adrenergic receptor blockers, HMG-CoA reductase inhibitors, angiotensin II receptor antagonists, angiotensin converting enzyme inhibitors, calcium channel blockers, endothelin antagonists, renin inhibitors, diuretics, aldosterone receptor blockers, endothelin receptor blockers, aldosterone synthase inhibitors, CETP inhibitor, relaxin, PCSK9 inhibitors, BNP and NEP inhibitors, GLP-1 analogues, insulin, sulphonylureas, biguanides, meglitinides, glucosidase inhibitors, glucagon antagonists, DPP-IV inhibitors, SGLT2 inhibitors. The treatment with the compound or pharmaceutically its pharmaceutically acceptable salt or the pharmaceutical composition of the present invention may also be combined with heart surgery.

A compound, in which -D-AB is a GLP-1 receptor agonist, or its pharmaceutically acceptable salt thereof or a pharmaceutical composition of the present invention may also be administered in combination with a growth hormone, such as a human growth hormone. Such human growth hormone may be in the form of an unmodified drug, such as the human growth hormone of SEQ ID NO:58, or as a conjugate or complex comprising human growth hormone. Suitably, the compound, in which -D-AB is a GLP-1 receptor agonist, is administered to a patient in a co-treatment with lonapegsomatropin, which has the following structure:

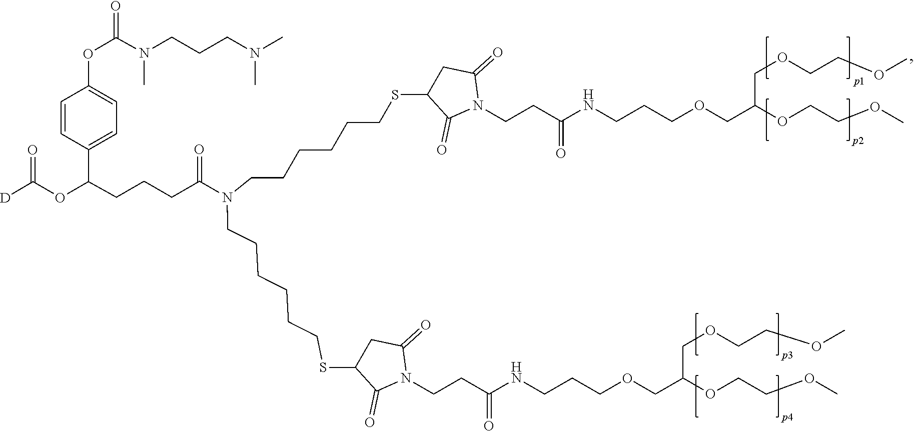

wherein
D is a human growth hormone polypeptide of SEQ ID NO:58 connected to the rest of the molecule through an amine functional group provided by a lysine side chain; and
each p1, p2, p3, p4 is independently an integer ranging from 210 to 240.

SEQ ID NO:58 has the following sequence:

FPTIPLSRLFDNAMLRAHRLHQLAFDTYQEFEEAYIPKEQKYSFLQNPQ
TSLCFSESIPTPSNREETQQKSNLELLRISLLLIQSWLEPVQFLRSVFA
NSLVYGASDSNVYDLLKDLEEGIQTLMGRLEDGSPRTGQIFKQTYSKFD
TNSHNDDALLKNYGLLYCFRKDMDKVETFLRIVQCRSVEGSCGF

In such co-treatment the compound, in which -D-AB is a GLP-1 receptor agonist, may be administered prior to, at the same time or after administration of the unmodified human growth hormone, the conjugate or complex comprising human growth hormone.

A compound, in which the corresponding free drug is a PTH, such as the compound of SEQ ID NO:52 or SEQ ID NO:53, or its pharmaceutically acceptable salt thereof or a pharmaceutical composition of the present invention may be used in the treatment or prevention of a disease selected from the group consisting of hypoparathyroidism, hyperphosphatemia, osteoporosis, fracture repair, osteomalacia, osteomalacia and osteoporosis in patients with hypophosphatasia, steroid-induced osteoporosis, male osteoporosis, arthritis, osteoarthritis, osteogenesis imperfecta, fibrous dysplasia, rheumatoid arthritis, Paget's disease, humoral hypercalcemia associated with malignancy, osteopenia, periodontal disease, bone fracture, alopecia, chemotherapy-induced alopecia, thrombocytopenia, chronic periodontitis, osteonecrosis of jaw and poorly healing fractures due to ALPL gene mutations.

In certain embodiments, said disease is hypoparathyroidism.

In certain embodiments, said disease is hypoparathyroidism induced by immune checkpoint inhibitor treatment. In certain embodiments, said disease is hypoparathyroidism induced by immune checkpoint inhibitor treatment targeting CTLA-4 (cytotoxic T-lymphocyte-associated protein 4), PD-1 (programmed cell death protein 1), PD-L1 (programmed death-ligand 1), PD-L² (programmed death-ligand 2), KIR (killer-cell immunoglobulin-like receptor), B7-H3, B7-H4, BTLA (B- and T-lymphocyte attenuator), LAG3 (lymphocyte-activation gene 3), TIM-3 (T-cell immunoglobulin and mucin-domain containing-3), VISTA (V-domain Ig suppressor of T cell activation), ILT2/LILRB1 (Ig-like transcript 2/leukocyte Ig-like receptor 1), ILT3/LILRB4 (Ig-like transcript 3/leukocyte Ig-like receptor 4), ILT4/LILRB2 (Ig-like transcript 4/leukocyte Ig-like receptor 2), TIGIT (T cell immunoreceptor with Ig and ITIM domains), NKG2A, PVRIG or combinations thereof.

In certain embodiments, said disease is hypoparathyroidism induced by immune checkpoint inhibitor treatment targeting CTLA-4, PD-1, PD-L1 or combinations thereof. In certain embodiments, said disease is hypoparathyroidism induced by immune checkpoint inhibitor treatment targeting CTLA-4 with immune checkpoint inhibitors such as ipilimumab, tremelimumab, MK-1308, FPT155, PRSO10, BMS-986249, BPI-002, CBT509, JS007, ONC392, TE1254, IBI310, BR02001, CG0161, KN044, PBI5D3H5, BCD145, ADU1604, AGEN1884, AGEN1181, CS1002 or CP675206. In certain embodiments, said disease is hypoparathyroidism induced by immune checkpoint inhibitor treatment targeting PD-1 with immune checkpoint inhibitors such as pembrolizumab, nivolumab, pidilizumab, AMP-224, BMS-936559, cemiplimab or PDR001. In certain embodiments, said disease is hypoparathyroidism induced by immune checkpoint inhibitor treatment targeting PD-L1 with immune checkpoint inhibitors such as MDX-1105, MEDI4736, atezolizumab, avelumab, BMS-936559 or durvalumab. In certain embodiments, said disease is hypoparathyroidism induced by immune checkpoint inhibitor treatment targeting PD-L2. In certain embodiments, said disease is hypoparathyroidism induced by immune checkpoint inhibitor treatment targeting KIR with immune checkpoint inhibitors such as lirilumab (IPH2102) or IPH2101. In certain embodiments, said disease is hypoparathyroidism induced by immune checkpoint inhibitor treatment targeting B7-H3 with immune checkpoint inhibitors such as MGA271. In certain embodiments, said disease is hypoparathyroidism induced by immune checkpoint inhibitor treatment targeting B7-H4 with immune checkpoint inhibitors such as FPA150. In certain embodiments, said disease is hypoparathyroidism induced by immune checkpoint inhibitor treatment targeting BTLA. In certain embodiments, said disease is hypoparathyroidism induced by immune checkpoint inhibitor treatment targeting LAG3 with immune checkpoint inhibitors such as IMP321 (eftilagimod alpha), relatlimab, MK-4280, AVA017, BI754111, ENUM006, GSK2831781, INCAGN2385, LAG3Ig, LAG525, REGN3767, Sym016, Sym022, TSR033, TSR075 or XmAb22841. In certain embodiments, said disease is hypoparathyroidism induced by immune checkpoint inhibitor treatment targeting TIM-3 with immune checkpoint inhibitors such as LY3321367, MBG453 or TSR-022. In certain embodiments, said disease is hypoparathyroidism induced by immune checkpoint inhibitor treatment targeting VISTA with immune checkpoint inhibitors such as JNJ-61610588. In certain embodiments, said disease is hypoparathyroidism induced by immune checkpoint inhibitor treatment targeting ILT2/LILRB1. In certain embodiments, said disease is hypoparathyroidism induced by immune checkpoint inhibitor treatment targeting ILT3/LILRB4. In certain embodiments, said disease is hypoparathyroidism induced by immune checkpoint inhibitor treatment targeting ILT3/LILRB2 with immune checkpoint inhibitors such as MK-4830. In certain embodiments, said disease is hypoparathyroidism induced by immune checkpoint inhibitor treatment targeting TIGIT with immune checkpoint inhibitors such as MK-7684, PTZ-201, RG6058 or COM902. In certain embodiments, said disease is hypoparathyroidism induced by immune checkpoint inhibitor treatment targeting NKG2A with immune checkpoint inhibitors such as IPH-2201. In certain embodiments, said disease is hypoparathyroidism induced by immune checkpoint inhibitor treatment targeting PVRIG with immune checkpoint inhibitors such as COM701. In certain embodiments, said disease is hypoparathyroidism induced by immune checkpoint inhibitor treatment targeting both PD-1 and CTLA-4. In certain embodiments, said disease is hypoparathyroidism induced by immune checkpoint inhibitor treatment with nivolumab. In certain embodiments, said disease is hypoparathyroidism induced by immune checkpoint inhibitor treatment with ipilimumab. In certain embodiments, said disease is hypoparathyroidism induced by immune checkpoint inhibitor treatment with pembrolizumab. In certain embodiments, said disease is hypoparathyroidism induced by immune checkpoint inhibitor treatment with a combination of nivolumab and ipilimumab.

In certain embodiments, said disease is rheumatoid arthritis induced by immune checkpoint inhibitor treatment. In certain embodiments, said disease is rheumatoid arthritis induced by immune checkpoint inhibitor treatment targeting PD-1 or PD-L1. In certain embodiments, said disease is rheumatoid arthritis induced by immune checkpoint inhibitor treatment targeting PD-1. In certain embodiments, said disease is rheumatoid arthritis induced by immune checkpoint inhibitor treatment targeting PD-L1. In certain embodiments, said disease is rheumatoid arthritis induced by immune checkpoint inhibitor treatment with nivolumab. In certain embodiments, said disease is rheumatoid arthritis induced by immune checkpoint inhibitor treatment with pembrolizumab. In certain embodiments, said disease is rheumatoid arthritis induced by immune checkpoint inhibitor treatment with a combination of nivolumab and ipilimumab. In certain embodiments, said disease is rheumatoid arthritis which recursed after immune checkpoint inhibitor treatment.

A compound or its pharmaceutically acceptable salt thereof or a pharmaceutical composition of the present invention may be administered, such as via subcutaneous administration, once a week, every two weeks, every three weeks or once a month.

In certain embodiments the compound or its pharmaceutically acceptable salt thereof or a pharmaceutical composition of the present invention may be administered once a week, every two weeks, every three weeks or once a month.

### Materials and Methods

### Chemicals

All materials were obtained from commercial vendors except where stated otherwise.

Semaglutide was purchased as the free acid from AdipoGen AG (Füllinsdorf, Switzerland). Tirzepatide hydrochloride was purchased from MedChemExpress (New Jersey, USA).

Pentaerythritol tetra{[3-3(3-maleimido-i-oxopropyl)amino]propyl}polyoxyethylene (alternative name: 4-arm 10 kDa-PEG tetramaleimide) was purchased from NOF Corporation. Bis-Mal-Lysine-PEG4-TFP ester, Amine-PEG-amine, MW 5,000 and Mal-PEG-Mal, MW 5,000 were purchased from Broadpharm, San Diego, CA.

mal-dPEG(3)-mal was purchased from Iris Biotech GmbH, Marktredwitz, Germany. 4-arm PEG-Mal, 5 kDa was purchased from Creative PEGWorks.

The number of ethylene glycol units “n” in a PEG reagent varies from batch to batch due to the polydispersity of polymeric moieties. All values for “n” are thus calculated based on the nominal molecular weight of a PEG reagent as provided by the manufacturer.

### Reactions

Generally, reactions were stirred at room temperature and monitored by LCMS.

### RP-HPLC Purification

Preparative RP-HPLC purifications were performed with a Waters 600 controller with a 2487 Dual Absorbance Detector, an Agilent 1260 Infinity II preparative system, or a Knauer Azura Preparative gradient system. A Waters XBridge BEH300 Prep C18 10 μm, 150×30 mm column or XSelect CSH Prep C18 10 μm 150×30 mm column were used as stationary phase.

Products were detected at 215 nm, 254 nm or 280 nm. Linear gradients of solvent system A (water containing 0.1% TFA v/v) and solvent system B (acetonitrile containing 0.1% TFA v/v) were used. HPLC fractions containing product were pooled and lyophilized if not stated otherwise.

### Flash Chromatography

Flash chromatography purifications were performed on an Isolera One system or an Isolera Four system from Biotage AB, Sweden using silica cartridges. Products were detected at 254 nm, 280 nm, or 360 nm.

### UPLC-MS Analysis

Analytical ultra-performance LC (UPLC)-MS was performed on a Waters Acquity system or an Agilent 1290 Infinity II equipped with a Waters BEH300 C₁₈ column (2.1×50 mm, 1.7 μm particle size or 2.1×100 mm, 1.7 μm particle size) or with a XSelect CSH C18 (2.1×50 mm, 2.5 μm particle size); solvent A: water containing 0.04% TFA (v/v), solvent B: acetonitrile containing 0.05% TFA (v/v) coupled to a Waters Micromass ZQ, Waters SQ Detector 2 or to an Agilent Single Quad MS system.

### NMR Analysis

NMR measurements were recorded using a 43 MHz Spinsolve Carbon benchtop (Magritek GmbH).

### EXAMPLE 1

### Preparation of [Lys(Boc)16]tirzepatide

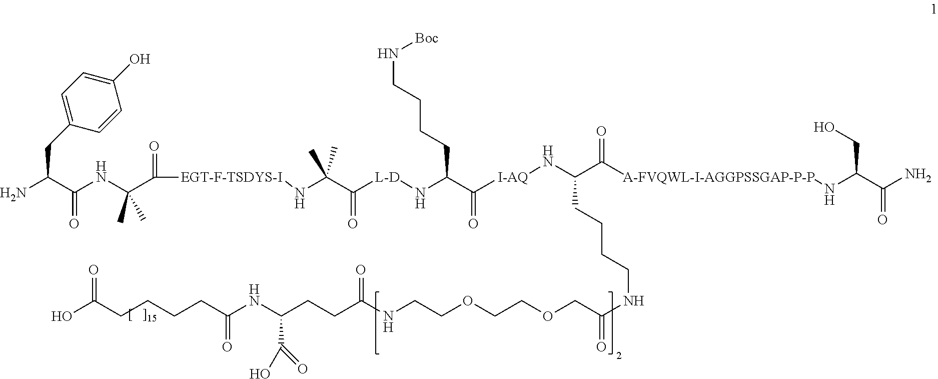

Tirzepatide hydrochloride (2.0 mg, 1.0 eq., 0.41 mol) was dissolved in water (19 μL), 1 M aq. NaHCO₃ solution (5.5 μL) and DMF (56 μL). To this was added N-(tert-butoxycarbonyloxy)succinimide (0.09 mg, 1.0 eq., 0.41 mol) in DMF (1.8 μL). The solution was shaken in an Eppendorf tube at rt for 160 min before quenching with 400 μL 1:1 acetonitrile/water (+0.1% TFA), TFA (1 μL) and water (200 μL). The crude product was then purified by preparative RP-HPLC to give 1 as a white solid as TFA salt.

Yield: 1.2 mg (58%, TFA salt)

MS: m/z 1638.91=[M+3H]³⁺, (calculated for [M+3H]³⁺=1638.53).

### EXAMPLE 2

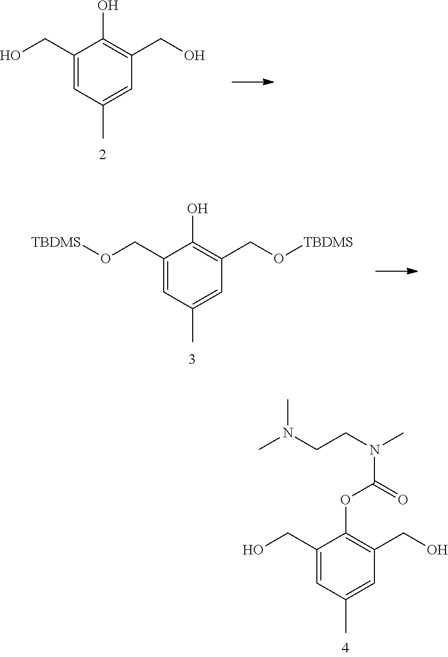

### Preparation of Compound 3

2,6-bis(hydroxymethyl)-p-cresol (300 mg, 1.0 eq., 1.78 mmol) and imidazole (304 mg, 2.5 eq., 4.46 mmol) were dissolved in DMF (3.00 mL) and placed in an ice-bath. TBDMS-Cl (672 mg, 2.5 eq., 4.46 mmol) was added. After stirring for 25 min the ice-bath was removed. A high amount of precipitate was formed and the addition of more DMF (3.00 mL) was sufficient to obtain a clear solution. After a further 55 mins, the reaction mixture was diluted with DCM (20 mL) and sat. aq. NH₄Cl. (20 mL). The aq. phase was extracted with DCM (2×15 mL). The combined organics were washed with sat. aq. NH₄Cl, dried over Na₂SO₄, and dried in vacuo.

Yield: 622 mg (88%)

¹H NMR (40 MHz, DMSO-d6): δ [ppm]=8.07 (s, 1H, Ar—OH), 6.97 (s, 2H, Ar), 4.70 (s, 4H, 2×CH₂), 2.21 (s, 3H, Me), 0.90 (s, 18H, 2×tBu), 0.08 (s, 12H, 4×Me)

### Preparation of Compound 4

Compound 3 (200 mg, 1.0 eq., 462 mol) was dissolved in THF (2.5 mL) and DIPEA (161 μL, 2.0 eq., 923 mol) was added. 4-nitrophenyl chloroformate (97.7 mg, 1.05 eq., 485 mol) was added and it was stirred at rt and a suspension was formed over time. After 45 min, trimethylethylenediamine (60 μL, 1 eq., 462 mol) was added and a yellow suspension was formed. After 30 min the reaction was quenched by addition of TFA (107 μL, 3.0 eq., 1.39 mmol) and all volatiles were evaporated. To the crude material was added 0.75 mL of acetonitrile/water 3:2+0.1% TFA. To the suspension was added TFA (107 μL) and after stirring at rt a solution formed. After stirring for 20 mins the deprotection was complete and the mixture was purified directly by preparative RP-HPLC. Compound 4 was obtained as TFA salt (a colourless oil).

Yield: 23 mg (12%, TFA salt)

MS: m/z, 297.12=[M+H]⁺, (calculated for [M+H]⁺=297.18).

### Preparation of Compound 5

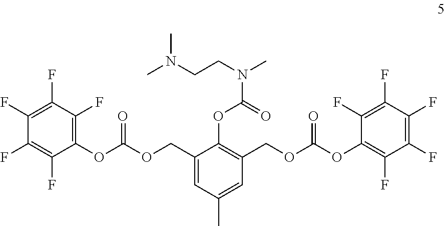 [SMILES: Cc1cc(COC(=O)Oc2c(F)c(F)c(F)c(F)c2F)c(OC(=O)N(C)CCN(C)C)c(COC(=O)Oc2c(F)c(F)c(F)c(F)c2F)c1]

Compound 4 (22.7 mg, 1.0 eq., 55.3 mol) was dissolved in acetonitrile (3.0 mL) and cooled in an ice bath. DMAP (1.4 mg, 0.2 eq., 11.1 mol), DIPEA (77.1 μL, 8.0 eq., 443 mol) and bis(pentafluorophenyl) carbonate (87.2 mg, 4.0 eq., 221 mol) were added. The mixture was stirred at rt for 25 min and changed color to dark green over time. The reaction was quenched with TFA (34 μL) and purified directly by preparative RP-HPLC to give 5 as TFA salt.

Yield: 38 mg (83%, TFA salt)

MS: m/z, 717.15=[M+H]⁺, (calculated for [M+H]⁺=717.13).

### EXAMPLE 3

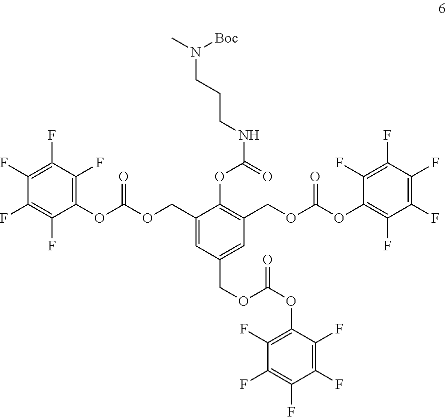 [SMILES: CN(CCCNC(=O)Oc1c(COC(=O)Oc2c(F)c(F)c(F)c(F)c2F)cc(COC(=O)Oc2c(F)c(F)c(F)c(F)c2F)cc1COC(=O)Oc1c(F)c(F)c(F)c(F)c1F)C(=O)OC(C)(C)C]

Compound 6 is prepared from the commercially available compounds (2-hydroxybenzene-1,3,5-triyl)trimethanol and 1,1-dimethylethyl N-methyl-N-[3-(methylamino)propyl]carbamate as per the protocol in example 2 except with an increase in reagent equivalents to account for the additional hydroxymethyl substituent on the aromatic ring.

### EXAMPLE 4

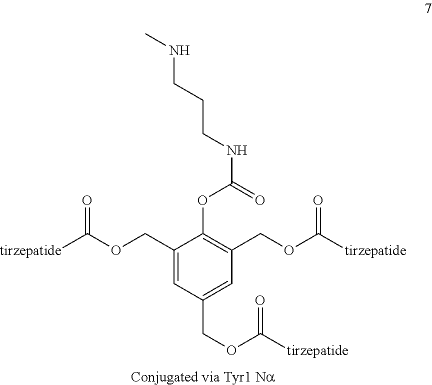

[Lys(Boc)16]tirzepatide 1 (4 eq.) in pH 7.0 phosphate buffer is treated with compound 6 (1 eq.) in DMF. After stirring for 1 h, the mixture is diluted with water and lyophilized. The crude mixture is treated with HFIP/TES/water/DTT (39/1/5/2.5) then TFA. The conjugate is isolated by precipitation with ice-cold ether and is then purified by preparative RP-HPLC to give 7 as the TFA salt.

### EXAMPLE 5

### Preparation of Compound 8

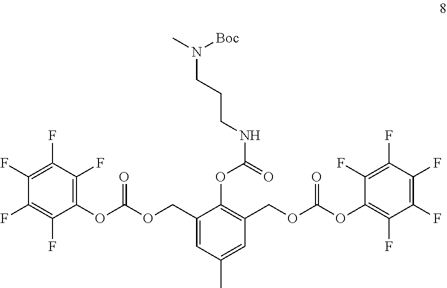 [SMILES: Cc1cc(COC(=O)Oc2c(F)c(F)c(F)c(F)c2F)c(OC(=O)NCCCN(C)C(=O)OC(C)(C)C)c(COC(=O)Oc2c(F)c(F)c(F)c(F)c2F)c1]

Compound 8 is prepared from 2,6-bis(hydroxymethyl)-p-cresol and 1,1-dimethylethyl N-methyl-N-[3-(methylamino)propyl]carbamate as per the procedure in example 2.

### Preparation of Compound 9

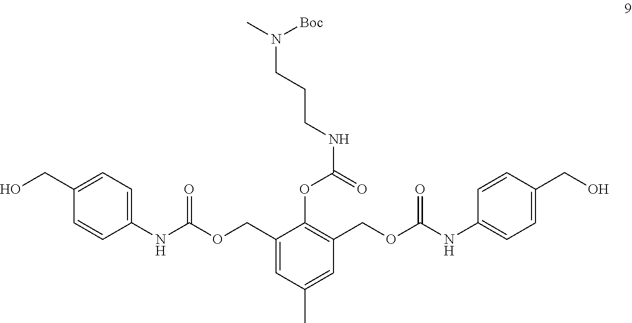 [SMILES: Cc1cc(COC(=O)Nc2ccc(CO)cc2)c(OC(=O)NCCCN(C)C(=O)OC(C)(C)C)c(COC(=O)Nc2ccc(CO)cc2)c1]

Compound 8 (1.0 eq.) in acetonitrile is treated with 4-(((tert-butyldimethylsilyl)oxy)methyl)aniline (2.5 eq.) and DIPEA (5.0 eq.). After stirring at rt for 1 h, the reaction is quenched by addition of TFA (6 eq.). A solution of acetonitrile/water 3:2+0.1% TFA is added to the solution. TFA is added to the mixture which is stirred until all alcohols have been deprotected. Compound 9 is purified by preparative RP-HPLC.

### Preparation of Compound 10

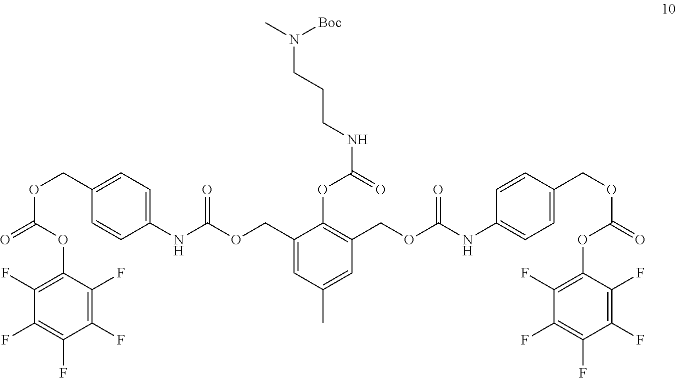 [SMILES: Cc1cc(COC(=O)Nc2ccc(COC(=O)Oc3c(F)c(F)c(F)c(F)c3F)cc2)c(OC(=O)NCCCN(C)C(=O)OC(C)(C)C)c(COC(=O)Nc2ccc(COC(=O)Oc3c(F)c(F)c(F)c(F)c3F)cc2)c1]

To 9 (1.0 eq.) in acetonitrile is added DMAP (0.2 eq.), DIPEA (8.0 eq) and bis(pentafluorophenyl) carbonate (4.0 eq.). The mixture is stirred at rt until complete then quenched with TFA. Compound 10 is purified directly by preparative RP-HPLC.

### EXAMPLE 6

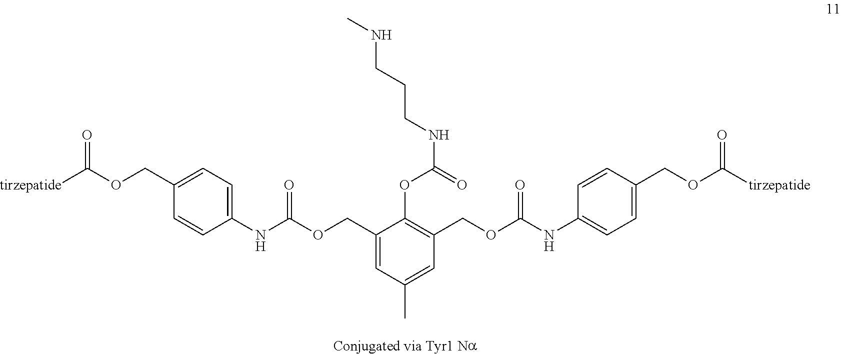

Compound 11 is prepared from 1 and 10 following the protocol described in example 4.

### EXAMPLE 7

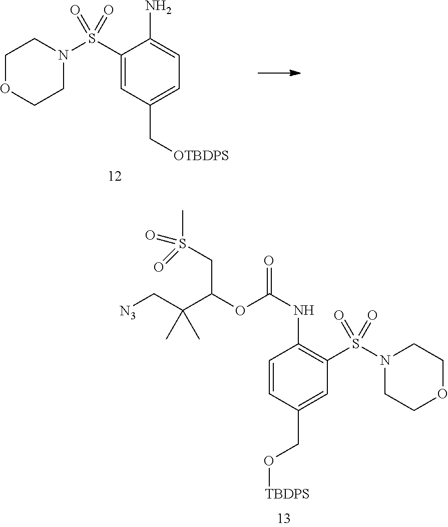

### Preparation of 12

Compound 12 is prepared as per the protocols described in WO2016085391 (example 9) except using morpholine instead of tert-butylamine to form the sulfonamide.

### Preparation of 13

Compound 13 is prepared from the aniline 12 and the compound 4-azido-1-(methylsulfonyl)-3,3-dimethyl-2-butyl succinimidyl carbonate (preparation described in example 1 of US20220280654) as per the protocol described in Bioconjugate Chemistry 2013, 24 (12), 1990-1997.

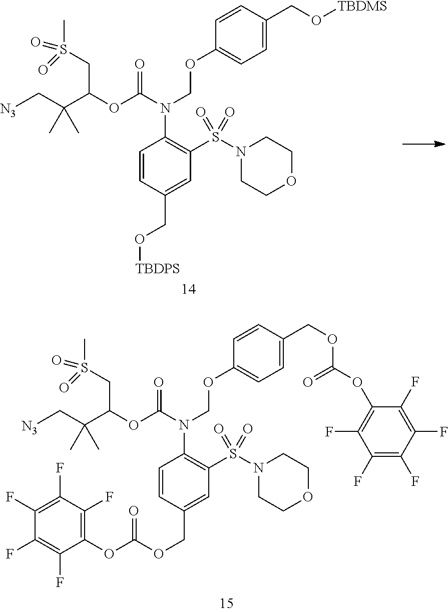

### Preparation of 14

Compound 14 is prepared from 13 and 4-[[[(1,1-dimethylethyl)dimethylsilyl]oxy]methyl]phenol using the protocol described in Bioconjugate Chemistry 2013, 24 (12), 1990-1997.

### Preparation of 15

Compound 14 is dissolved in MeOH and treated with acetyl chloride (0.15 eq.). The solution is stirred at rt until all alcohol protecting groups have been removed. The intermediate compound is purified by preparative RP-HPLC. The alcohols are reacted with bis(pentafluorophenyl) carbonate using the procedure described in example 2 to give compound 15.

### EXAMPLE 8

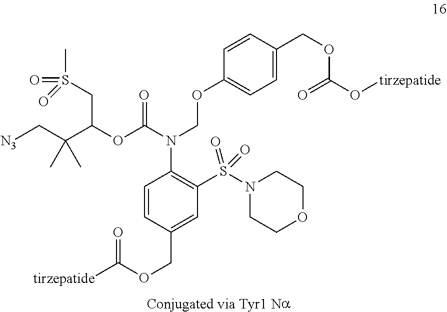

Compound 16 is prepared from 1 and 15 following the protocol described in example 4.

### EXAMPLE 9

### Preparation of 17

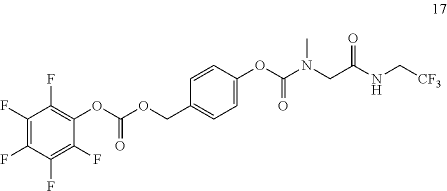 [SMILES: CN(CC(=O)NCC(F)(F)F)C(=O)Oc1ccc(COC(=O)Oc2c(F)c(F)c(F)c(F)c2F)cc1]

Compound 17 is prepared from the commercially available amine 2-(methylamino)-N-(2,2,2-trifluoroethyl)acetamide and 4-hydroxybenyl alcohol as per the protocol described in example 2.

### Preparation of 19

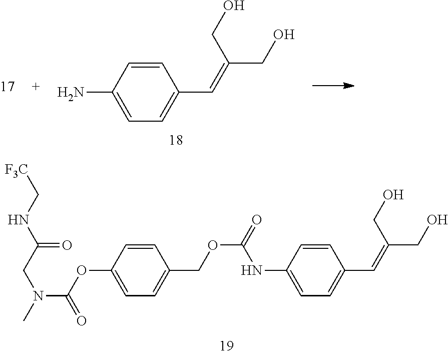 [SMILES: Nc1ccc(C=C(CO)CO)cc1 ; CN(CC(=O)NCC(F)(F)F)C(=O)Oc1ccc(COC(=O)Nc2ccc(C=C(CO)CO)cc2)cc1]

To compound 17 in DMF is added compound 18 (1.1 eq. and prepared as described in example 6 of WO9813059) and DIPEA (2 eq.). A catalytic amount of hydroxybenzotriazole monohydrate (0.4 eq.) is added and the mixture stirred at rt. After the reaction is complete, it is quenched by addition of TFA and compound 19 is purified by preparative RP-HPLC.

### Preparation of 20

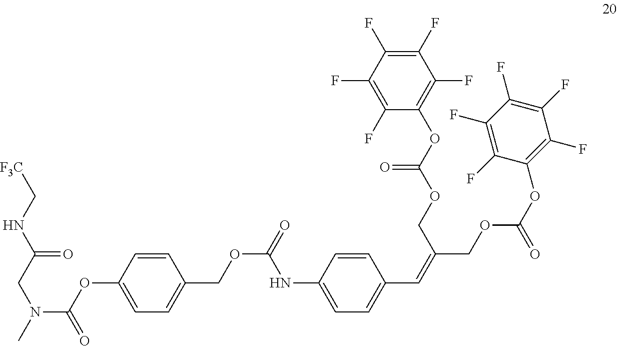 [SMILES: CN(CC(=O)NCC(F)(F)F)C(=O)Oc1ccc(COC(=O)Nc2ccc(C=C(COC(=O)Oc3c(F)c(F)c(F)c(F)c3F)COC(=O)Oc3c(F)c(F)c(F)c(F)c3F)cc2)cc1]

Compound 20 is prepared from compound 19 as per the protocol in example 2.

### Preparation of 21

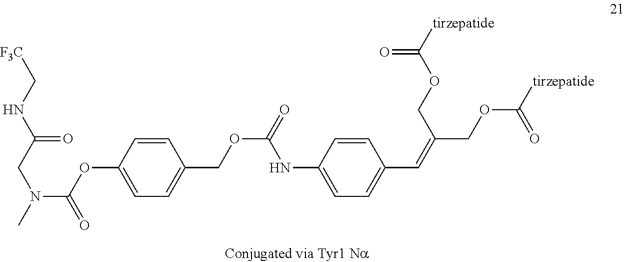

Compound 21 is prepared from compounds 1 and 20 as per the protocol described in example 4.

### EXAMPLE 10

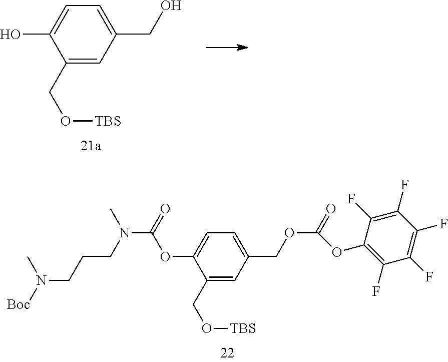

### Preparation of 21a

Compound 21a is prepared as described in Organic & Biomolecular Chemistry 2009, 7 (23), 4825-4828.

### Preparation of 22

Compound 22 is prepared from 21 and 1,1-dimethylethyl N-methyl-N-[3-(methylamino)propyl]carbamate by following the procedure described in WO2016196124 (example 16).

### Preparation of 24

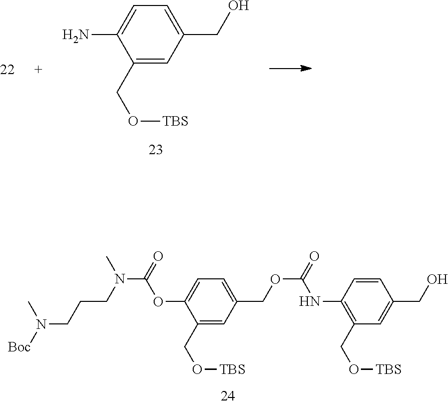

Compound 24 is prepared from compound 23 and compound 22 using the procedure described for the preparation of compound 19 in example 9.

### Preparation of 25

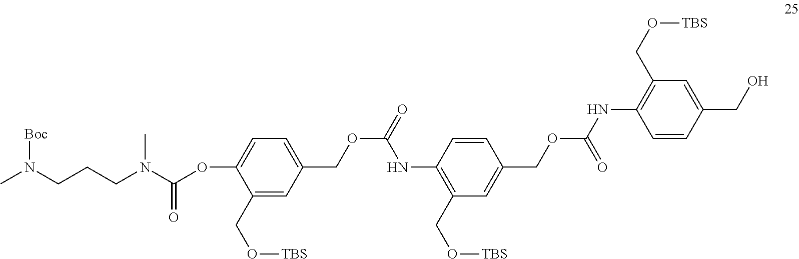

The free alcohol of 24 is activated with bis(pentafluorophenyl) carbonate using the protocol described in example 2. This intermediate is then coupled with 23 using the procedure described for the preparation of compound 19 in example 9 to give compound 25.

### Preparation of 26

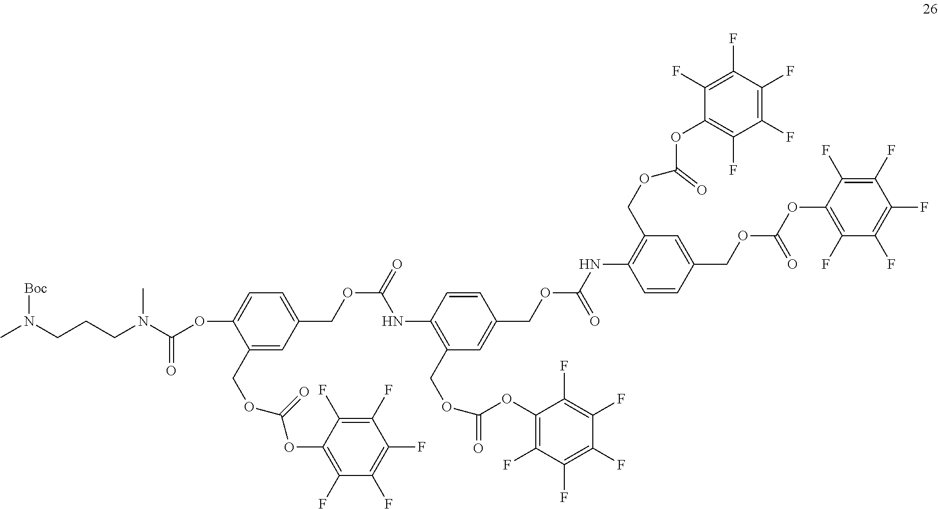 [SMILES: CN(CCCN(C)C(=O)OC(C)(C)C)C(=O)Oc1ccc(COC(=O)Nc2ccc(COC(=O)Nc3ccc(COC(=O)Oc4c(F)c(F)c(F)c(F)c4F)cc3COC(=O)Oc3c(F)c(F)c(F)c(F)c3F)cc2COC(=O)Oc2c(F)c(F)c(F)c(F)c2F)cc1COC(=O)Oc1c(F)c(F)c(F)c(F)c1F]

Compound 25 dissolved in a solution of 3:2 acetonitrile/water+0.1% TFA. Further TFA is carefully added to the mixture until all alcohols have been deprotected. The deprotected intermediate is purified by preparative RP-HPLC. The intermediate (1.0 eq.) is dissolved in acetonitrile, cooled in an ice bath, and is treated with DMAP (0.4 eq.), DIPEA (12.0 eq.) and bis(pentafluorophenyl) carbonate (8.0 eq.). The mixture is stirred at rt until the reaction is complete, then quenched with TFA and purified by preparative RP-HPLC to give 26.

### EXAMPLE 11

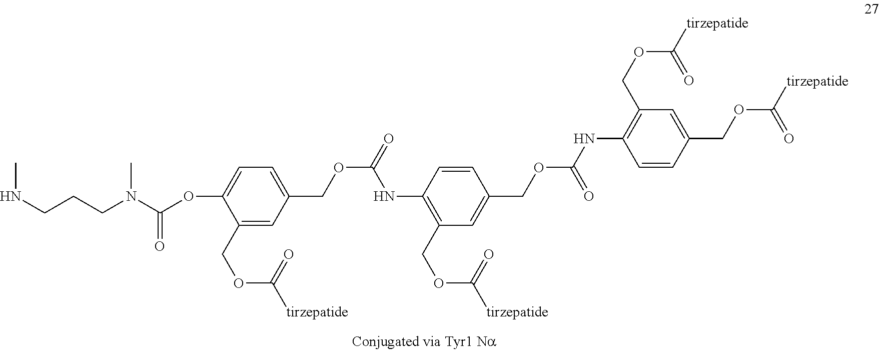

Compound 27 is prepared from 1 and 26 following the protocol described in example 4 except with a higher excess of compound 1.

### EXAMPLE 12

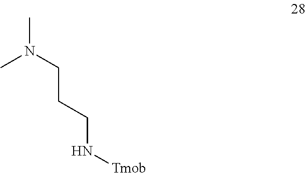

Compound 28 was prepared following the procedure described in WO2020064847 (example 11).

MS: m/z, 283.15=[M+H]⁺, (calculated [M+H]⁺=283.20).

### EXAMPLE 13

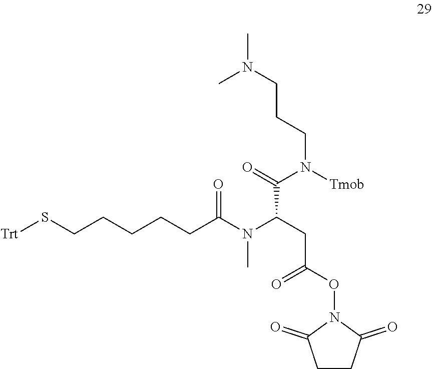

Compound 29 was prepared as the TFA salt from 28 according to the procedure described in WO2020064847 (example 11).

MS: m/z, 881.36=[M+H]⁺, (calculated [M+H]⁺=881.42).

### EXAMPLE 14

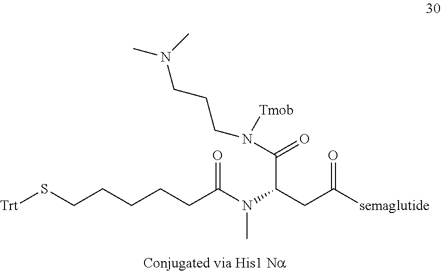

Semaglutide (5 mg, 1.0 eq., 1.22 mol) was dissolved in a mixture of 100 mM pH 8 borate buffer (0.5 mL) and acetonitrile (0.5 mL). To this was added 29 (7.3 mg, 6.0 eq., 7.29 mol) dissolved in acetonitrile (73 μL). After stirring at rt for 21 h, the reaction was quenched by addition of 10% aq. TFA (20 μL) and purified directly by preparative RP-HPLC to give 30 as the TFA salt.

Yield: 1.6 mg (25%, TFA salt)

MS: m/z 1627.74=[M+3H]³⁺, (calculated [M+3H]³⁺=1627.17).

### EXAMPLE 15

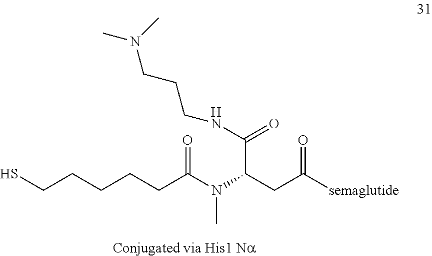

The protected conjugate 30 (1.6 mg, 1.0 eq., 0.30 mol) was treated with 80 μL of HFIP/TES/water/DTT (39/1/5/2.5) then 20 μL of TFA. The mixture was shaken at rt for 55 min before addition of 1 mL of ice-cold diethyl ether. The mixture was vortexed, centrifuged, and the supernatant removed. The resulting precipitate was washed twice more with 1 mL of ice-cold diethyl ether. The crude conjugate was then purified via preparative RP-HPLC to give 31 as the TFA salt.

Yield: 1.1 mg (75%. TFA salt)

MS: m/z 1486.94=[M+3H]³⁺, (calculated [M+3H]³⁺=1486.45).

### EXAMPLE 16

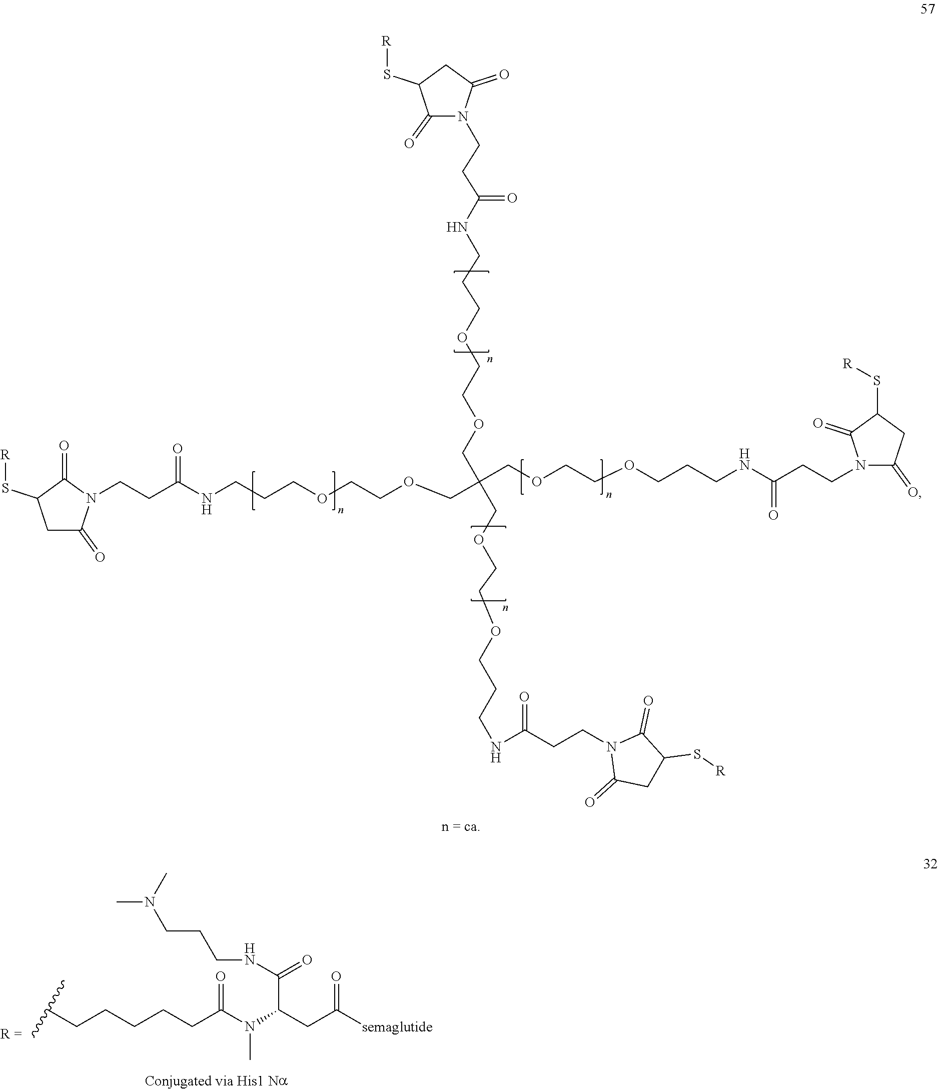

To a solution of 4-arm 10 kDa-PEG tetramaleimide (0.38 mg, 1.0 eq., 0.037 μmol) in 250 mM pH 7.4 phosphate buffer (38 μL) was added conjugate 31 (0.37 mg, 2.0 eq, 0.075 μmol) in 250 mM pH 7.4 phosphate buffer (50 μL), acetonitrile (50 μL), and water (25 μL). After shaking at rt for 20 mins at rt, conjugate 31 (0.37 mg, 2.0 eq, 0.075 μmol) in 250 mM pH 7.4 phosphate buffer (50 μL), acetonitrile (50 μL), and water (25 μL) were added to the reaction mixture. After a further 20 mins shaking at rt, conjugate 31 (0.19 mg, 1.0 eq, 0.037 μmol) in 250 mM pH 7.4 phosphate buffer (25 μL). acetonitrile (25 μL), and water (13 μL) were added to the reaction mixture. After a further 95 mins, the reaction was quenched by addition of TFA (5 μL) and the mixture was purified by preparative RP-HPLC to give 32 as a TFA salt.

Yield: 0.6 mg (54%, TFA salt)

### EXAMPLE 18

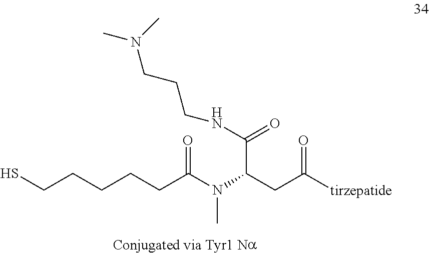

Conjugate 34 is prepared from 1 as per example 14 and 15.

### EXAMPLE 19

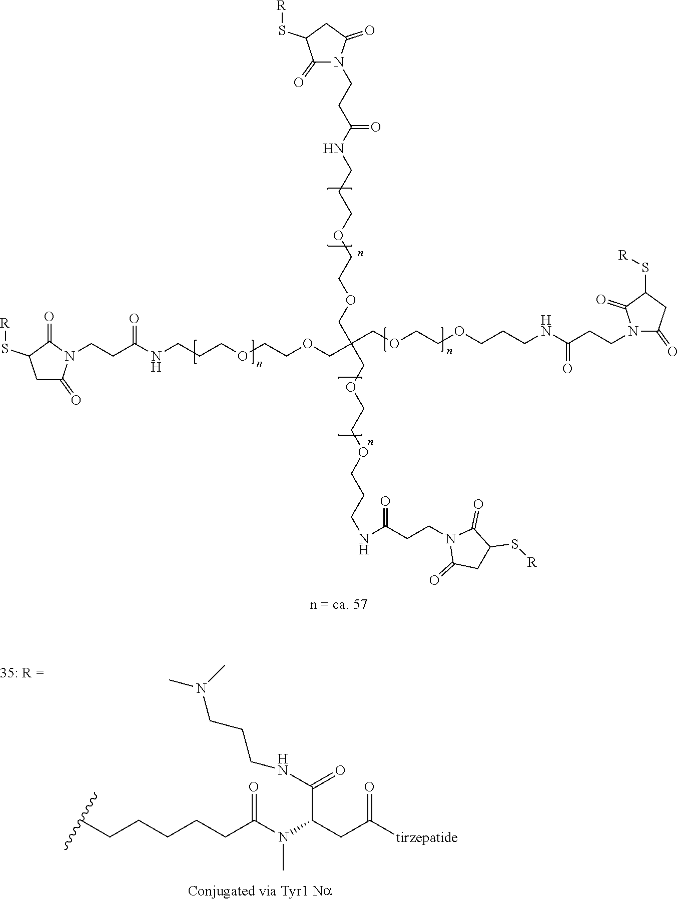

Compound 35 is prepared from 34 and 4-arm 10 kDa-PEG tetramaleimide as per example 16.

### EXAMPLE 20

### Maleimide Functionalized Hyaluronic Acid

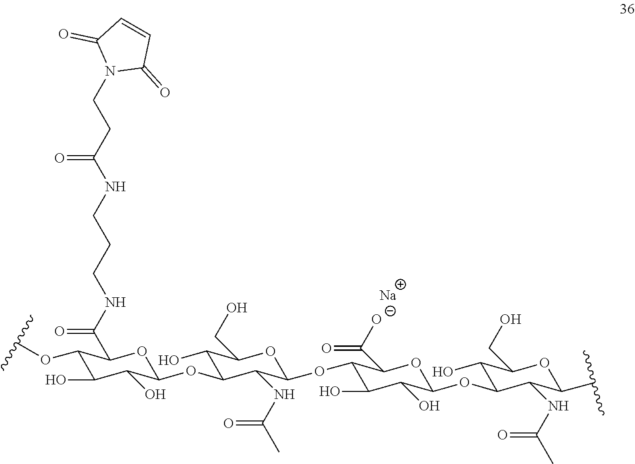 [SMILES: *OC1C(C(=O)NCCCNC(=O)CCN2C(=O)C=CC2=O)OC(OC2C(O)C(CO)OC(OC3C(C(=O)[O-])OC(OC4C(O)C(CO)OC(*)C4NC(C)=O)C(O)C3O)C2NC(C)=O)C(O)C1O ; [Na+]]

Maleimide functionalized hyaluronic acid 36 is prepared from hyaluronic acid (8 kDa) as described in WO2018175788 (example 2B). A degree of functionalization is achieved that gives between 2 and 6 maleimide groups per molecule.

### EXAMPLE 21

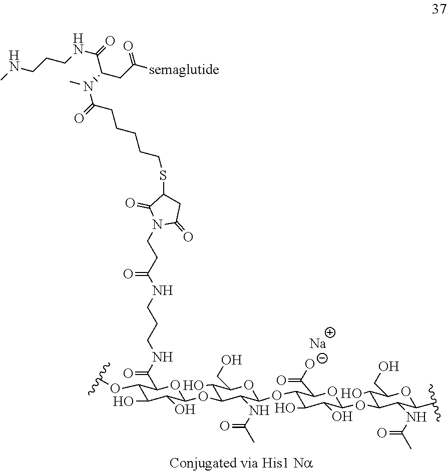

Maleimide functionalized hyaluronic acid 36 is conjugated to compound 31 as per example 16, except that the resulting conjugate 37 is purified by centrifugal filtration using a membrane with a 5 kDa molecular weight cut-off.

### EXAMPLE 22

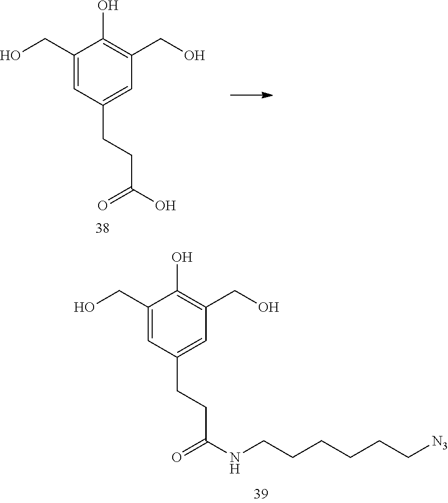 [SMILES: O=C(O)CCc1cc(CO)c(O)c(CO)c1 ; [N-]=[N+]=NCCCCCCNC(=O)CCc1cc(CO)c(O)c(CO)c1]

### Compound 38:

Compound 38 is prepared as described in U.S. Pat. No. 3,689,540 (example 1).

### Compound 39:

Compound 39 is prepared by coupling compound 38 with 6-azidohexan-1-amine using HATU and DIPEA in DMF.

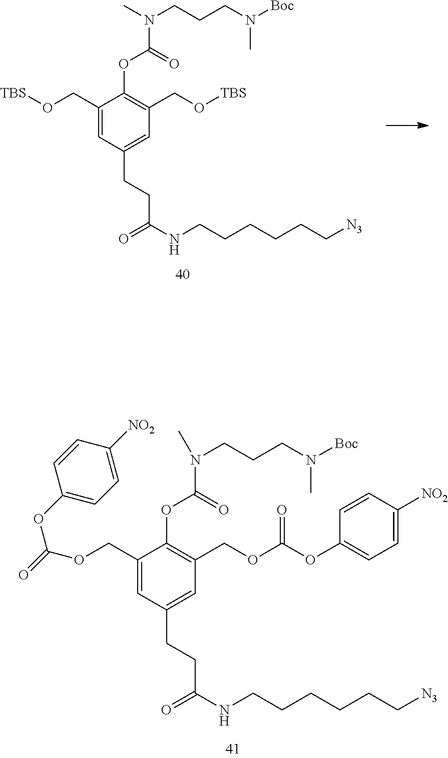

### Compound 40:

Compound 40 is prepared from 39 and 1,1-dimethylethyl N-methyl-N-[3-(methylamino)propyl]carbamate using a protocol similar to that described in Angewandte Chemie International Edition 2005, 44 (5), 716-720.

### Compound 41:

Compound 41 is prepared from 40 using a protocol similar to that described in Angewandte Chemie International Edition 2005, 44 (5), 716-720.

### EXAMPLE 23

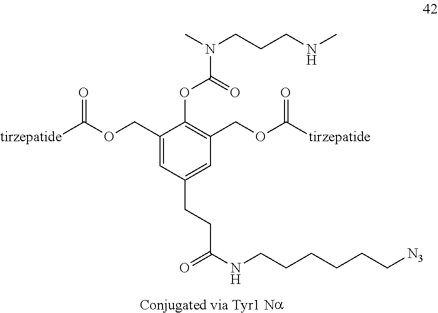

To [Lys(Boc)16]tirzepatide 1 (2.25 eq.) in pH 7.0 phosphate buffer is added compound 41 in DMF. After stirring for 1 h, the mixture is diluted with water and lyophilized. The crude mixture is treated with HFIP/TES/water/DTT (39/1/5/2.5) then TFA. The conjugate is isolated by precipitation with ice-cold ether and is then purified by preparative RP-HPLC to give 42.

### EXAMPLE 24

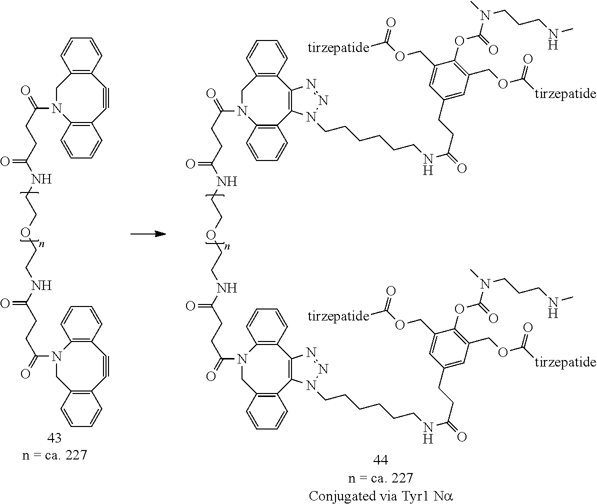

To the commercially available bis-functional 10 kDa PEG 43 is added 42 in 1:1 buffer/DMF. After stirring at rt for 2 h, the crude material is purified directly by preparative RP-HPLC to give 44.

### EXAMPLE 25

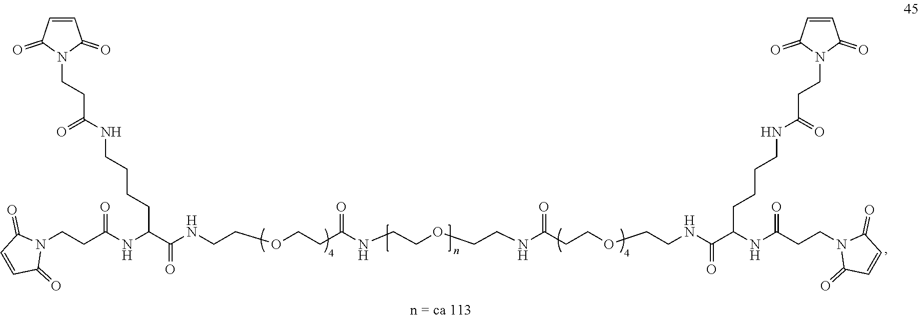

Bis-Mal-Lysine-PEG4-TFP ester (20.8 mg, 2.5 eq, 24.7 mol) was dissolved in 1 mL acetonitrile/water (3:1 v/v). Amine-PEG-amine, MW 5,000 (50.0 mg, 1 eq, 9.88 μmol) dissolved in 1 mL acetonitrile/water (3:1 v/v) was added, followed by DIPEA (2.55 mg, 3.44 μL. 2 Eq. 19.8 μmol). The clear solution was stirred at rt. After 30 min the reaction was quenched with 3 μl TFA and the product purified by preparative RP-HPLC to give 45 as colorless solid.

Yield: 35 mg (55%)

### EXAMPLE 26

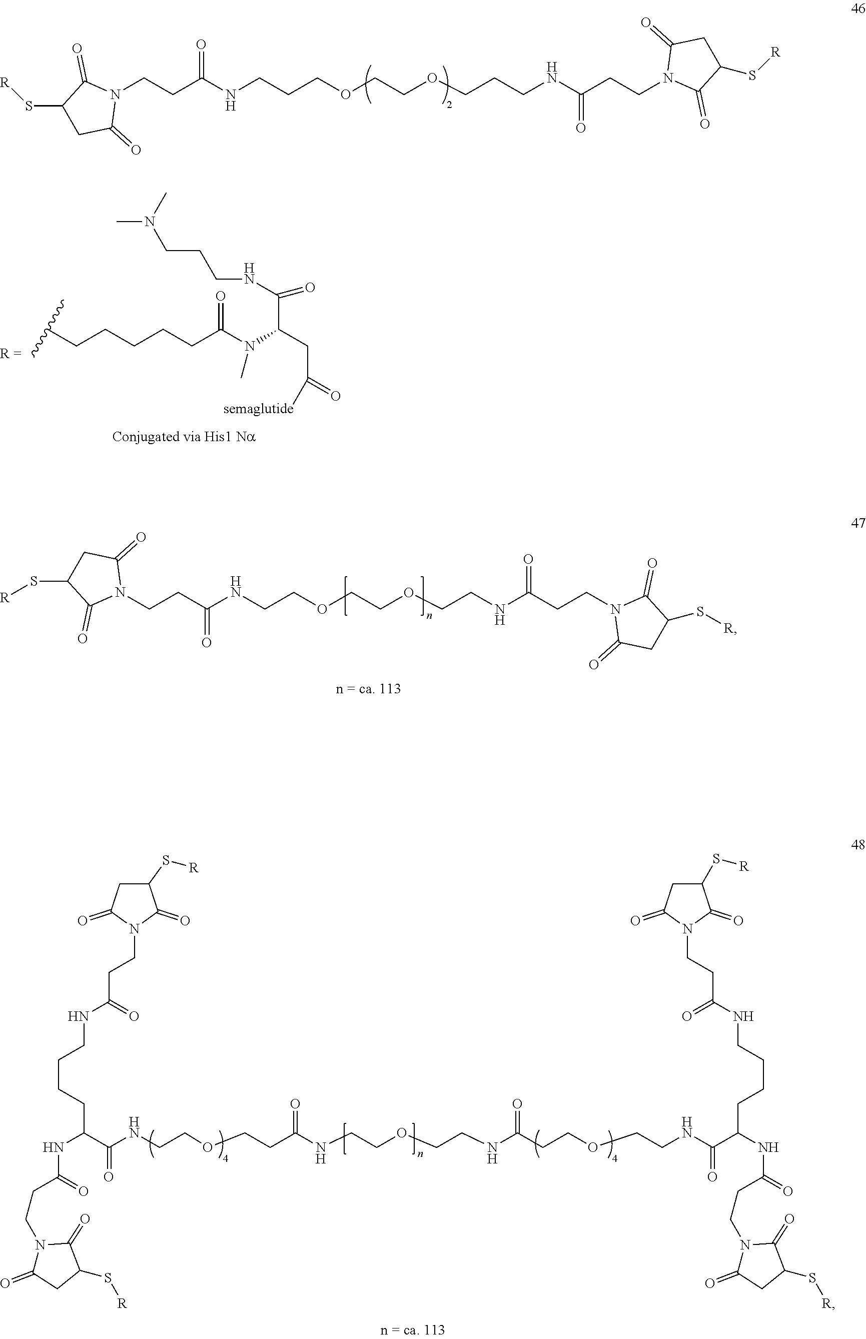

An excess of 31 in acetonitrile/water was added to 1 eq of the respective PEG maleimide portion-wise. The pH of the reaction was adjusted by addition of pH 7.0 or 7.4 sodium phosphate buffer. The reaction solution was shaken at rt and monitored by LC/MS. Upon completion the reaction was acidified with TFA. The product was purified by preparative RP-HPLC.

Synthesis of 46: 1 eq mal-dPEG(3)-mal (0.60 mg),
2.2 eq 31 (11.0 mg), pH 7.4: yield: 8.80 mg (75%, 8×TFA salt),
MS: m/z 1349.31=[M+7H]⁷+, (calculated [M+7H]⁷⁺=1349.12)

Synthesis of 47: 1 eq Mal-PEG-Mal, MW 5,000 (11.7 mg),
2.0 eq 31 (21.4 mg), pH 7.0: yield: 18.4 mg (56%, 8×TFA salt)

Synthesis of 48: 1 eq 45 (2.90 mg), 4.5 eq 31(10.0 mg), pH 7.4: yield: 7.10 mg (60%, 16×TFA salt)

### EXAMPLE 27: PK STUDY IN SPRAGUE DAWLEY RATS

The objective of the study was to determine the pharmacokinetic (PK) characteristics of 32 when administered once by the intravenous (IV) or the subcutaneous (SC) route to the adult male Sprague Dawley rat. A semaglutide IV and SC group was included as comparator.

The Study design is shown in Table 1. Blood samples were processed to plasma and provided for PK analysis of semaglutide released from 32.

TABLE 1
Design of Study
(Dose is in mg semaglutide equivalents/kg)

| | | | | Admin- | Blood sampling |
|---|---|---|---|---|---|
| | | | Dose | istration | timepoints (hours |
| Group | Test item | n | (mg/kg) | Route | post dose) |
| 1 | Semaglutide | 3 | 0.030 | IV | Predose, 0.25, 0.5, |
| | | | | | 1, 2, 4, 6, 24, 48 |
| 2 | Semaglutide | 3 | 0.030 | SC | Predose, 1, 3, 4, 6, |
| | | | | | 8, 12, 24, 48 |
| 3 | 32 | 3 | 0.222 | IV | 0.25, 3, 8, 24, 36, |
| | | | | | 48, 72, 168, 264 |
| 4 | 32 | 3 | 0.222 | SC | 3, 6, 10, 24, 36, |
| | | | | | 48, 72, 168, 264 |

### EXAMPLE 28: PK STUDY IN SPRAGUE DAWLEY RATS

The objective of the study was to determine the PK characteristics of 46 and 47 when administered once by the IV or the SC route to the adult male Sprague Dawley rat. A semaglutide SC group was included as comparator and a vehicle SC group was included as control.

The Study design is shown in Table 2. Blood samples were processed to plasma and provided for PK analysis of released semaglutide (except from group F).

TABLE 2
Design of Study
(Dose is in mg semaglutide equivalents/kg)

| | | | Dose | Administration | |
|---|---|---|---|---|---|
| Group | Test item | n | (mg/kg) | Route | Blood sampling timepoints (hours post dose) |
| A | 47 | 3 | 0.45 | IV | 0.25, 3, 8, 24, 36, 48, 72, 120, 168 and 264 |
| B | 46 | 3 | 0.45 | IV | 0.25, 3, 8, 24, 36, 48, 72, 120, 168 and 264 |
| C | 47 | 3 | 0.45 | SC | 3, 6, 10, 24, 36, 48, 72, 96, 168 and 264 |
| D | 46 | 3 | 0.45 | SC | 3, 6, 10, 24, 36, 48, 72, 96, 168 and 264 |
| E | Semaglutide | 3 | 0.030 | SC | 1, 2, 4, 6, 8, 12, 24, 48 and 58 |
| F | Vehicle control | 3 | 0 | SC | 3, 6, 10, 24, 36, 48, 72, 96, 168 and 264 |

### EXAMPLE 29: PK ANALYSIS OF SEMAGLUTIDE RELEASED FROM COMPOUNDS 32, 46 AND 47 COMPARED TO SEMAGLUTIDE AND PK ANALYSIS OF SEMAGLUTIDE RELEASED FROM 49 AND 51 IN SPRAGUE DAWLEY RATS

The PK of semaglutide and semaglutide released from 32, 46, 47, 49 or 51 was determined after IV or SC administration of semaglutide and of 32, 46, 47, 49 or 52 to rats according to the study described in example 27, example 28 and example 40.

Blood samples were collected from all animals according to Table 1, Table 2 and Table 8. Plasma samples were analyzed for semaglutide.

Semaglutide and semaglutide released from 32, 46, 47, 49 and 51 was quantified in Sprague Dawley rat K₃EDTA plasma via LC-MS/MS after plasma protein precipitation. Samples containing semaglutide were pipetted into the wells of a 96-well plate, followed by the addition of internal standard (d₈-semaglutide). Plasma protein precipitation was carried out using a mixture of acetonitrile and water (85/15, v/v). The precipitate was centrifuged at 4° C. and a fraction of the supernatant was transferred to a new 96-well plate, evaporated to dryness under a stream of heated nitrogen and reconstituted in water containing 0.2% formic acid.

Chromatography was performed on a Waters Acquity UPLC Peptide BEH C₁₈ analytical column (1.7 μm particle size; pore size 300 Å; column dimensions 50×2.1 mm). Water (UPLC grade) containing 0.1% formic acid (v/v) was used as mobile phase A and acetonitrile (UPLC grade) with 0.1% formic acid as mobile phase B. Multiple reaction monitoring (MRM) was performed for semaglutide and the internal standard and quantification was based on peak area and linear regression 1/x² weighting.

A summary of the mean PK parameters for semaglutide and semaglutide released from 32, 46, 47, 49 and 51 in rat plasma are shown below in Table 3, Table 4 and Table 5.

TABLE 3
Summary of the Mean PK Parameters of semaglutide and semaglutide
released from 32 in rat plasma in study described in example 27.

| | | | Dose | | | | | |
|---|---|---|---|---|---|---|---|---|
| Dose | Test | | level | Cₘₐₓ | tₘₐₓ | tₗₐₛₜ | AUCₗₐₛₜ | t_{1/2} |
| group | Item | ROA | (μg/kg) | (ng/mL)^{a} | (h)^{b} | (h)^{b} | (h*ng/mL)^{a} | (h)^{a} |
| 1 | semaglutide | IV | 30 | 570^{c} | 0.25 | 48 | 5,400^{c} | 10^{c} |
| 2 | semaglutide | SC | 30 | 130 ± 26 | 6 | 48 | 3,000 ± 620 | 11 ± 1.3 |
| 3 | 32 | IV | 222 | 34 ± 1.3 | 24 | 168 | 2,500 ± 660 | 43 ± 2 |
| 4 | 32 | SC | 222 | 24 ± 3 | 24 | 72 | 1,100 ± 120 | 30 ± 0.9 |

^{a}mean value ± SD;
^{b}median value;
^{c}n = 2

TABLE 4
Summary of the mean PK parameters of semaglutide and semaglutide
released from 46 or 47 in rat plasma in example 28.

| Dose | Test | | Dose level | Cₘₐₓ | tₘₐₓ | tₗₐₛₜ | AUCₗₐₛₜ | t_{1/2} |
|---|---|---|---|---|---|---|---|---|
| group | Item | ROA | (μg/kg) | (ng/mL)^{a} | (h)^{b} | (h)^{b} | (h*ng/mL)^{a} | (h)^{a} |
| A | 47 | IV | 450 | 56 ± 7.1 | 24 | 120 | 3,400 ± 250 | 28 ± 2.2 |
| B | 46 | IV | 450 | 37 ± 0.9 | 8 | 72 | 1,800 ± 60 | 23 ± 2.1 |
| C | 47 | SC | 450 | 50 ± 1 | 24 | 96 | 2,500 ± 460 | 27 ± 2.6 |
| D | 46 | SC | 450 | 40 ± 5.2 | 24 | 96 | 1,700 ± 320 | 20 ± 1.9 |
| E | semaglutide | SC | 30 | 110 ± 21 | 8 | 58 | 3,100 ± 560 | 12 ± 0.2 |

^{a}mean value ± SD;
^{b}median value
Note:
group F served as vehicle control and was not dosed with an active compound

TABLE 5
Summary of the Mean PK Parameters of semaglutide and semaglutide released
from 49 and 51 in rat plasma in study described in example 40.

| | | | Dose | | | | | |
|---|---|---|---|---|---|---|---|---|
| Dose | Test | | level | Cₘₐₓ | tₘₐₓ | tₗₐₛₜ | AUCₗₐₛₜ | t_{1/2} |
| group | Item | ROA | (μg/kg) | (ng/mL)^{a} | (h)^{b} | (h)^{b} | (h*ng/mL)^{a} | (h)^{a} |
| 1 | 49 | IV | 450 | 58 ± 3.0 | 24 | 120 | 3,800 ± 170 | 29 ± 1.0 |
| 2 | 51 | IV | 450 | 35 ± 1.8 | 8 | 72 | 1,300 ± 190 | 18 ± 0.96 |

^{a}mean value ± SD;
^{b}median value

### EXAMPLE 30: PK STUDY IN CYNOMOLGUS MONKEY

The objective of the study was to determine the PK characteristics of 32 when administered once by the IV or SC route to the Cynomolgus monkey.

The Study design is shown in Table 6. Blood samples were processed to plasma and provided for PK analysis of semaglutide released from 32.

TABLE 6
Design of Study
Dose is in mg semaglutide equivalents/kg

| | | No. of | | | | |
|---|---|---|---|---|---|---|
| Group | Test | Animals | | Dose | Administration | Blood sampling time points |
| No. | item | Males | Females | (mg/kg) | Route | (hours post dose) |
| 1 | 32 | 1 | 1 | 0.15 | IV | Predose, 0.25, 2, 6, 12, 24, |
| | | | | | | 48, 72, 96, 120, 144, 168, |
| | | | | | | 216, 264, 288 and 336 |
| 2 | 32 | 1 | 1 | 0.15 | SC | Predose, 3, 6, 10, 16, 24, 36, |
| | | | | | | 48, 72, 96, 120, 144, 168, |
| | | | | | | 216, 264, 288 and 336 |

### EXAMPLE 31: PK ANALYSIS OF SEMAGLUTIDE RELEASED FROM 32 IN CYNOMOLGUS MONKEYS

The PK of semaglutide released from 32 was determined after IV or SC administration of 32 to monkeys in the study described in example 30.

Semaglutide released from 32 was quantified in Cynomolgus monkey K₃EDTA plasma via LC-MS/MS after plasma protein precipitation. Samples containing semaglutide were pipetted into the wells of a 96-well plate, followed by the addition of internal standard (d₈-semaglutide) solution containing 4% formic acid. Plasma protein precipitation was carried out using a mixture of acetonitrile and water (85/15, v/v). The precipitate was centrifuged at 4° C. and a fraction of the supernatant was transferred to a new 96-well plate, evaporated to dryness under a stream of heated nitrogen and reconstituted in water containing 0.2% formic acid.

Chromatography was performed on a Waters Acquity UPLC Peptide BEH C18 analytical column (1.7 μm particle size; pore size 300 Å; column dimensions 50×2.1 mm). Water (UPLC grade) containing 0.1% formic acid (v/v) was used as mobile phase A and acetonitrile (UPLC grade) with 0.1% formic acid as mobile phase B. Multiple reaction monitoring (MRM) was performed for semaglutide and the internal standard and quantification was based on peak area and linear regression with 1/x² weighting.

A summary of the individual and mean PK parameters for semaglutide released from 32 in monkey plasma are shown below in Table 7.

No sex-specific differences between the male and female animal within each group was observed. Cₘₐₓ and area under the curve (AUC) were slightly lower for the SC group than for the IV group, but the half-life was comparable in both groups.

TABLE 7
Summary of the individual and mean PK parameters of semaglutide released
from 32 in monkey Plasma in study described in example 30.

| | | | Dose | | | | | | |
|---|---|---|---|---|---|---|---|---|---|
| Dose | Test | | level | | Cₘₐₓ | tₘₐₓ | tₗₐₛₜ | AUCₗₐₛₜ | t_{1/2} |
| group | Item | ROA | (μg/kg) | Animal | (ng/mL) | (h) | (h) | (h*ng/mL) | (h) |
| 1 | 32 | IV | 150 | m | 84 | 72 | 336 | 19,000 | 123 |
| | | | | f | 92 | 72 | 336 | 20,000 | 123 |
| | | | | mean | 88 | | | 20,000 | 123 |
| | | | | median | | 72 | 336 | | |
| 2 | 32 | SC | 150 | m | 68 | 120 | 336 | 16,000 | 144 |
| | | | | f | 71 | 72 | 336 | 16,000 | 130 |
| | | | | mean | 69 | | | 16,000 | 137 |
| | | | | median | | 96 | 336 | | |

### EXAMPLE 32

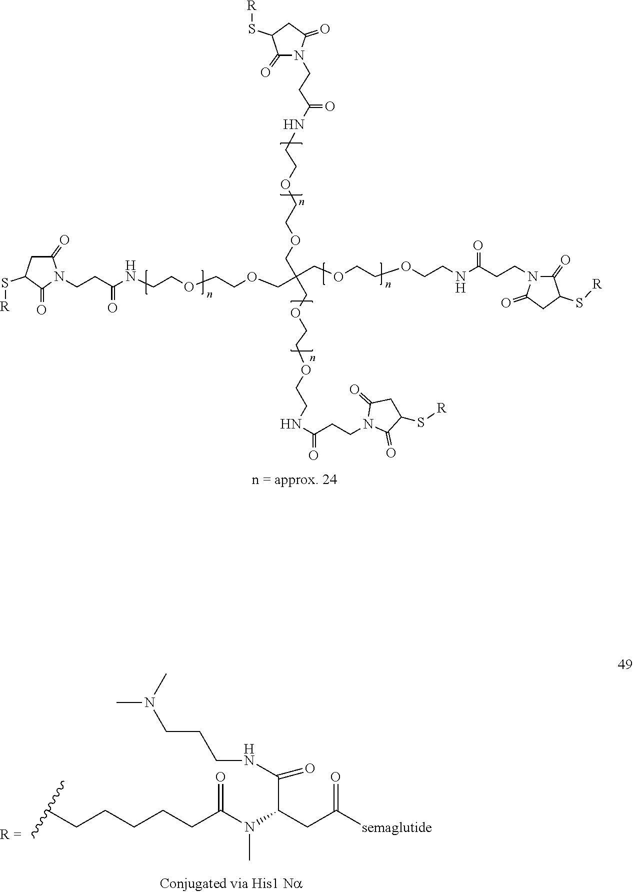

To a solution of 4-arm 5 kDa-PEG tetramaleimide (5.0 mg, 1.0 eq., 0.98 μmol) in acetonitrile (150 μL) was added conjugate 31(4.8 mg, 1.0 eq, 0.98 μmol) in 1:1 acetonitrile/water (200 μL) followed by 250 mM pH 7.4 phosphate buffer (200 μL). After shaking at rt for 20 min, conjugate 31 (9.6 mg, 2.0 eq, 1.96 mmol) in 1:1 acetonitrile/water (400 μL) was added. After a further 15 min shaking at rt, conjugate 31 (7.2 mg, 1.5 eq, 1.47 mmol) in 1:1 acetonitrile/water (300 μL) was added followed by additional 1:1 acetonitrile/water (3 mL). After a further 75 min, the reaction was quenched by addition of 10% TFA in water (50 μL) and the mixture was purified by preparative RP-HPLC to give 49 as a TFA salt.

Yield: 10.2 mg (42%)

### EXAMPLE 33

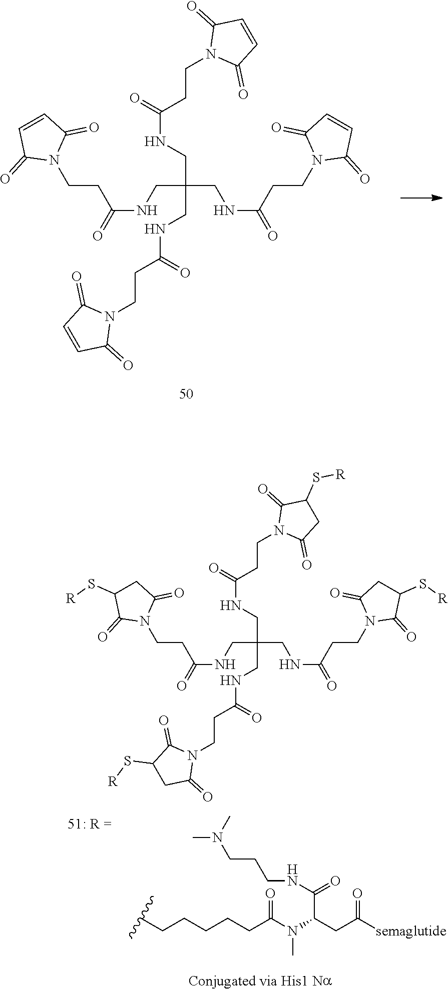

### Compound 50:

2,2-bis(aminomethyl)propane-1,3-diamine tetrahydrochloride (10.0 mg, 1.0 eq, 36.0 mol) was dissolved in acetonitrile (2.0 mL), DIPEA (23.2 mg, 31.3 μL, 5.0 eq, 180 mol) and water (0.4 mL). 3-(Maleimido)propionic acid N-hydroxysuccinimide ester (47.9 mg, 5.0 eq, 180 μmol) was added, followed by DIPEA (23.2 mg, 31.3 μL, 5.0 eq, 180 mol). After stirring at rt for 25 min, the reaction was quenched by addition of TFA (20 μL), and the mixture was purified by preparative RP-HPLC to give 50.

Yield: 23.8 mg (90%)

MS: m/z, 737.48=[M+H]⁺, (calculated [M+H]⁺=737.25).

### Compound 51:

To a solution of conjugate 31 (30.0 mg, 4.5 eq, 6.11 mol) in 1:1 acetonitrile/water (2.0 mL) was added a solution of compound 23 (1.00 mg, 1 eq, 1.36 mol) in acetonitrile (0.1 mL), followed by 250 mM pH 7.4 phosphate buffer (0.5 mL). After stirring at rt for 45 min, the reaction was quenched by addition of TFA (5 μL). The mixture was diluted with 1:1 acetonitrile/water (1 mL) and 250 mM pH 7.4 phosphate buffer (0.5 mL) and purified by preparative RP-HPLC to give 51 as a TFA salt.

Yield: 16.6 mg (60%)

MS: m/z, 1857.80=[M+10H]¹⁰⁺, (calculated [M+10H]¹⁰⁺=1857.52).

### EXAMPLE 34

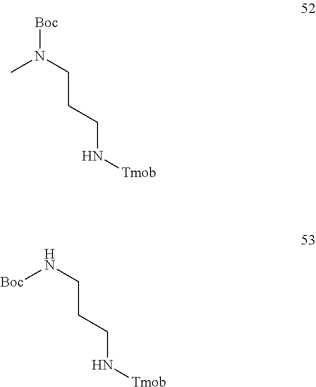

A solution of the respective amine (1.5 eq, see below) in acetic acid was added to a solution of 2,4,6-trimethoxybenzaldehyde (1.0 eq) in acetic acid. The reaction solution was stirred at rt and monitored by LCMS. After complete imine formation an excess of sodium borohydride was added portion wise. The reaction solution was stirred at rt and monitored by LCMS. After the reaction was complete it was quenched by addition of 1 N HCl. The mixture was stirred for 20 min and then added into water. The aqueous phase was washed with dichloromethane. The pH of the aqueous phase was adjusted to 14 using 12.5 N NaOH and extracted with dichloromethane. The organic phase was dried over sodium sulfate, evaporated and dried under high vacuum.

Synthesis of 52: 1.0 eq tert-butyl N-(3-aminopropyl)-N-methylcarbamate (1.41 g), 1.2 eq sodium borohydride (229 mg), yield: 2.14 g (100%, 86% purity), m/z 369.42=[M+H]⁺, (calculated [M+H]⁺=369.47).

Synthesis of 53 1.0 eq N-Boc-1,3-diaminopropane (1.31 g), 1.5 eq sodium borohydride (286 mg), yield: 2.16 g (100%, 82% purity), m/z 355.40=[M+H]⁺, (calculated [M+H]⁺=355.45).

### EXAMPLE 35

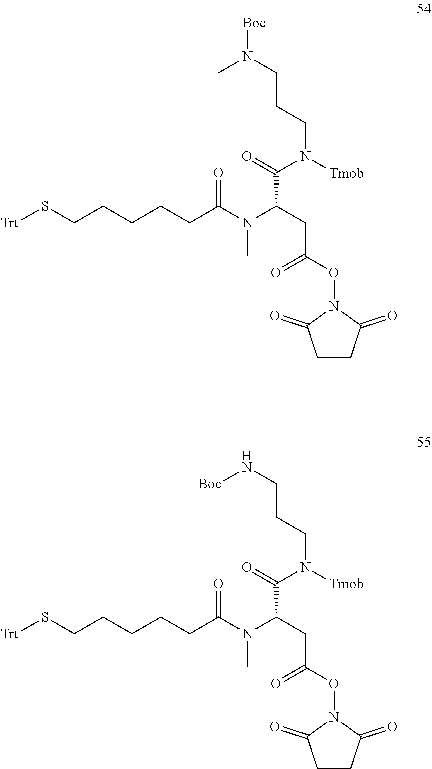

Compound 54 was prepared according to example 13 using compound 52 instead of 28

MS: m/z, =967.95 [M+H]⁺, (calculated [M+H]⁺=968.19).

Compound 28 was prepared according to example 13 using 53 instead of 28

MS: m/z, =953.90 [M+H]⁺, (calculated [M+H]⁺=954.16).

### EXAMPLE 36

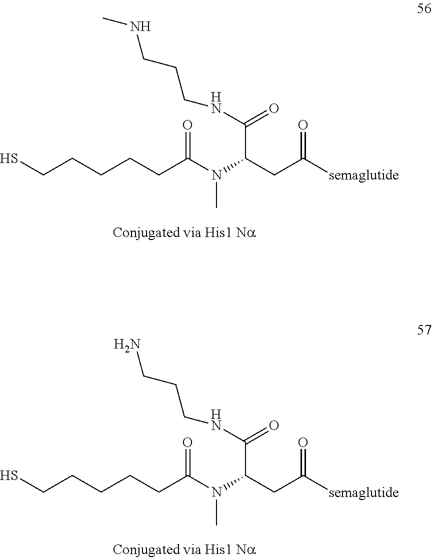

Compound 56 was prepared as TFA salt according to examples 14 and 15 starting from compound 54 instead of 29.

MS: m/z, =1481.92 [M+3H]³⁺, (calculated [M+3H]³⁺=1482.03).

Compound 57 was prepared as TFA salt according to examples 14 and 15 starting from compound 55 instead of 29.

MS: m/z, =1477.97 [M+3H]³⁺, (calculated [M+3H]³⁺=1477.36).

### EXAMPLE 37

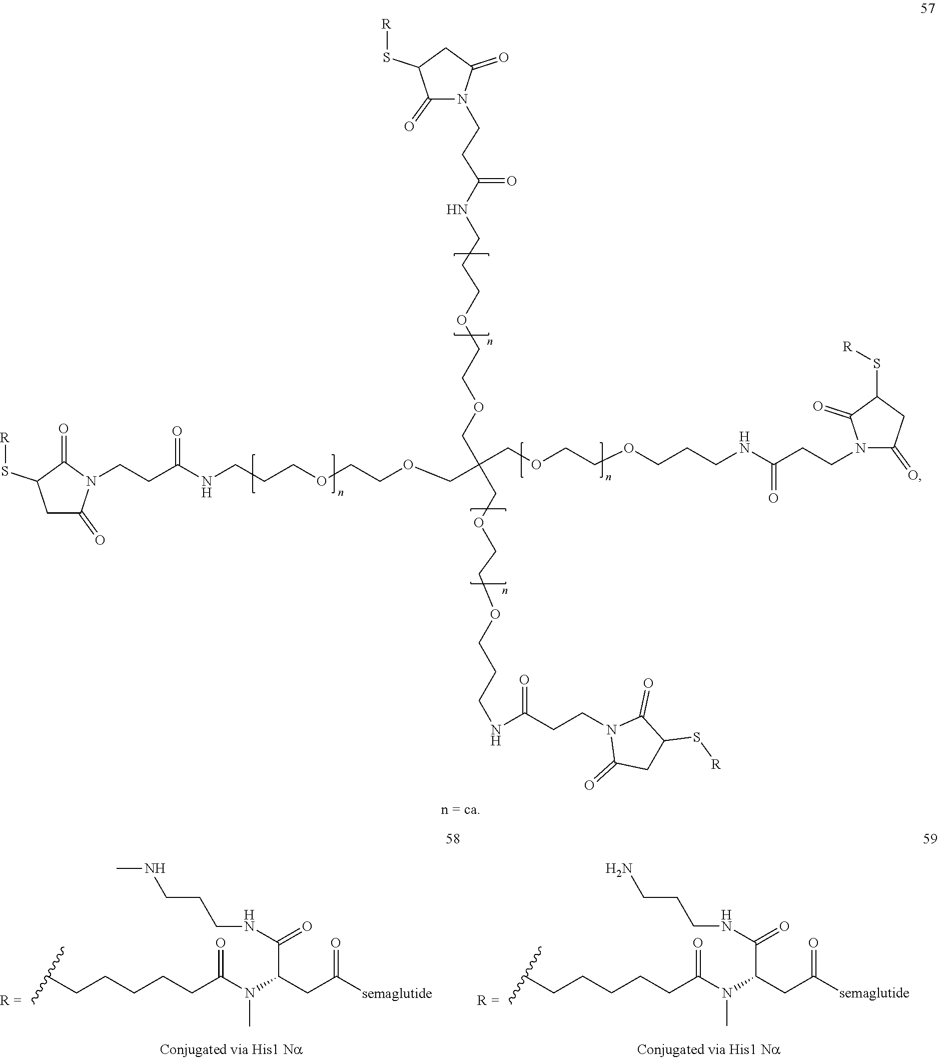

A solution of 4-arm 10 kDa-PEG tetramaleimide (1.0 eq) in acetonitrile was added to a solution of the respective conjugate (4.5 eq, 56 or 57) in 1:1 acetonitrile/water. 250 mM pH 7.4 phosphate buffer was added and the reaction solution was stirred at rt and monitored by LCMS. Upon completion the reaction was acidified by addition of TFA and purified by preparative RP-HPLC.

Synthesis of 58: 4.5 eq 56 (2.0 mg), yield: 1.0 mg (35%, TFA salt)

Synthesis of 59: 4.5 eq 54 (2.6 mg), yield: 3.9 mg (57% o, TFA salt)

### EXAMPLE 38

### Alternative Synthesis of Compound 34

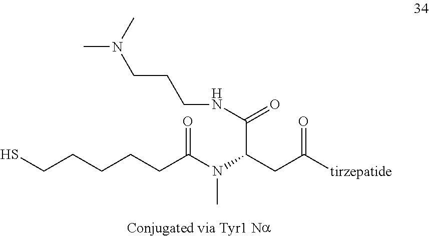

[Lys(Boc)16]tirzepatide 1 (1.2 mg, 1.0 eq, 0.24 μmol) was dissolved in a mixture of 120 μL acetonitrile and 120 μL 60 mM pH 8 phosphate buffer. To this was added a solution of 29 (1.43 mg, 6.0 eq, 1.43 μmol) in acetonitrile (14.3 μL). After stirring at rt for 2 d the solution was diluted with 1:1 acetonitrile/water+0.1% TFA (250 μL) and quenched with TFA (1 μL). The crude mixture was purified by preparative RP-HPLC to give of the protected conjugate as TFA salt.

Yield: 0.5 mg (36%),

MS: m/z 1421.07=[M+4H]⁴⁺, (calculated [M+4H]⁴⁺=1420.91).

The protected conjugate (0.5 mg, 1.0 eq, 0.086 μmol) was dissolved in HFIP/TES/water/DTT (39/1/5/2.5) (80 μL) and TFA (20 μL) was added. After stirring for 65 min the product was precipitated by addition of 1 mL ice-cold diethyl ether. The precipitate was washed with ice-cold diethyl ether and purified by preparative RP-HPLC to give 34 as the TFA salt.

Yield: 0.2 mg (43%),

MS: m/z 1720.15=[M+3H]³⁺, (calculated [M+3H]⁴⁺=1720.00).

### EXAMPLE 39

### Alternative Synthesis of Compound 35

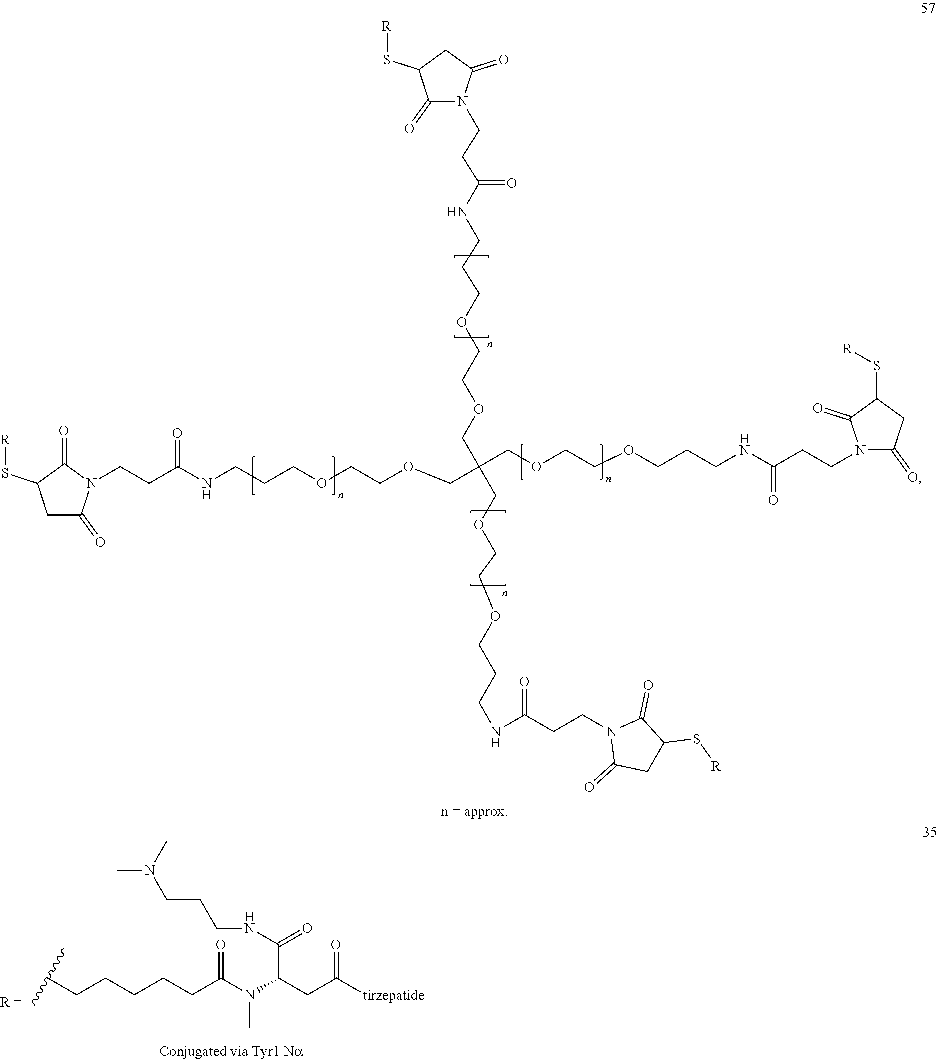

A solution of 34 (0.2 mg, 4.5 eq, 0.037 μmol) in 1:1 acetonitrile/water (225 μL) was prepared. 100 μL of the solution of 34 (2.0 eq) were added to a solution of 4-arm 10 kDa-PEG tetramaleimide (91 pig, 1.0 eq, 8.3 nmol) in 1:1 acetonitrile/water (9.1 μL), followed by 250 mM pH 7.4 phosphate buffer (10 μL). After shaking at rt for 15 min, 100 μL of the solution of 34 (2.0 eq) were added. After a further 15 min shaking at rt, 25 μL of the solution of 34 (0.5 eq) were added. After a further 50 min, the reaction was quenched by addition of TFA (1 μL), and the mixture was purified by preparative RP-HPLC to give 35 as a TFA salt.

### EXAMPLE 40: PK Study in Sprague Dawley Rats

The objective of the study was to determine the PK characteristics of 49 and 51 when administered once by the IV route to the adult male Sprague Dawley rat.

The Study design is shown in Table 8. Blood samples were processed to plasma and provided for PK analysis of released semaglutide.

TABLE 8
Design of Study
(Dose is in mg semaglutide equivalents/kg)

| | | | | Blood sampling |
|---|---|---|---|---|
| | Test | | Dose | timepoints |
| Group | item | n | (mg/kg) | (hours post dose) |
| 1 | 49 | 3 | 0.45 | 0.25, 3, 8, 24, 36, 48, |
| | | | | 60, 72, 120 and 168 |
| 2 | 51 | 3 | 0.45 | 0.25, 3, 8, 24, 36, 48, |
| | | | | 60, 72, 120 and 168 |

### Abbreviations:

aq. aqueous
AUC area under the curve
Ar aromatic
Boc tert-butyloxycarbonyl
DCM dichloromethane
DIPEA diisopropylethylamine
DMAP dimethylaminopyridine
DMF dimethylformamide
DMSO-d6 hexadeuterodimethyl sulfoxide
dPEG discrete PEG
DTT dithiothreitol
eq equivalent
HATU O-(7-Azabenzotriazol-1-yl)-N,N,N′,N′-tetramethyluronium-hexafluorphosphat
HFIP 1,1,1,3,3,3-hexafluoroisopropanol
HPLC high performance liquid chromatography
IV intravenous
LCMS liquid chromatography mass spectrometry
mal maleimide
MS mass spectrometry
Me methyl
MeOH methanol
MS mass spectrometry
PEG polyethylene glycol
PK pharmacokinetic
ROA Route of administration
RP reversed phase
rt room temperature
sat. saturated
SC subcutaneous
SD standard deviation
TBDMS tert-butyldimethylsilyl
TBDPS tert-butyldiphenylsilyl
TBS tert-butyldimethylsilyl
tBu and t-Bu tert-butyl
TES triethylsilane
TFA trifluoroacetic acid
TFP tetrafluorophenyl
THE tetrahydrofurane
Tmob 2,4,6-Trimethoxybenzyl
Trt trityl
UPLC ultra performance liquid chromatography
UPLC-MS ultra performance liquid chromatography coupled to mass spectrometry

1. A compound or a pharmaceutically acceptable salt thereof, wherein the compound comprises at least two covalently and reversibly conjugated drug moieties, each of the at least two drug moieties comprises an albumin-binding moiety, and wherein the compound releases the drug moieties as their corresponding free drug molecules.

2. The compound or a pharmaceutically acceptable salt thereof of claim 1, wherein the half-life of the drug released from the compound is at least 1.5-fold higher than the corresponding free drug's half-life.

3. The compound or a pharmaceutically acceptable salt thereof of claim 1, wherein the half-life of the drug released from the compound is at least 2.5-fold higher than the corresponding free drug's half-life.

4. The compound or a pharmaceutically acceptable salt thereof of claim 1, wherein the half-life of the drug released from the compound is at least 5-fold higher than the corresponding free drug's half-life.

5. The compound or a pharmaceutically acceptable salt thereof of claim 1, wherein the half-life of the drug released from the compound is at least 7.5-fold higher than the corresponding free drug's half-life.

6. The compound or a pharmaceutically acceptable salt thereof of claim 1, wherein the half-life of the drug released from the compound is at least 10-fold higher than the corresponding free drug's half-life.

7. The compound or a pharmaceutically acceptable salt thereof of claim 1, wherein the release half-life of the compound is at least the circulation half-life of the corresponding free drug.

8. The compound or a pharmaceutically acceptable salt thereof of claim 1, wherein the release half-life of the compound is at least 2-fold higher than the circulation half-life of the corresponding free drug.

9. The compound or a pharmaceutically acceptable salt thereof of claim 1, wherein the release half-life of the compound is at least 3-fold higher than the circulation half-life of the corresponding free drug.

10. The compound or a pharmaceutically acceptable salt thereof of claim 1, wherein the distance between any two moieties -AB of a compound is such that they are capable of binding to two different albumin molecules or to two different binding sites on the same albumin moiety.

11. The compound or a pharmaceutically acceptable salt thereof of claim 1, wherein the distance between any two moieties -AB of a compound is such that they are capable of binding to two different albumin molecules.

12. The compound or a pharmaceutically acceptable salt thereof of claim 1, wherein the distance between any two moieties -AB of a compound is such that they are capable of binding to two different binding sites on the same albumin moiety.

13. A pharmaceutical composition comprising at least one compound of claim 1 and at least one excipient.

14. (canceled)

15. A method of treating a patient, the method comprising the step of administering a pharmaceutically effective amount of the compound of claim 1 to the patient.


SEQUENCE LISTING (58 sequences)

SEQ ID NO: 1 - protein, 31 aa, Homo sapiens
HAEGTFTSDVSSYLEGQAAKEFIAWLVKGRG

SEQ ID NO: 2 - protein, 31 aa, synthetic construct
  SITE 2: note = Aib
HXEGTFTSDVSSYLEGQAAKEFIAWLVRGRG

SEQ ID NO: 3 - protein, 31 aa, synthetic construct; identical to SEQ ID NO: 2
  SITE 2: note = Aib
  MOD_RES 31: note = C-terminus is amidated

SEQ ID NO: 4 - protein, 39 aa, synthetic construct
HGEGTFTSDLSKQMEEEAVRLFIEWLKNGGPSSGAPPPS

SEQ ID NO: 5 - protein, 39 aa, synthetic construct; identical to SEQ ID NO: 4
  MOD_RES 39: note = C-terminus is amidated

SEQ ID NO: 6 - protein, 44 aa, synthetic construct
  MOD_RES 44: note = C-terminus is amidated
HGEGTFTSDLSKQMEEEAVRLFIEWLKNGGPSSGAPPSKKKKKK

SEQ ID NO: 7 - protein, 44 aa, synthetic construct; identical to SEQ ID NO: 6

SEQ ID NO: 8 - protein, 39 aa, synthetic construct
  SITE 2: note = D-Ala
  SITE 14: note = Nle
HAEGTFTSDLSKQXEEEAVRLFIEWLKQGGPSSGAPPPC

SEQ ID NO: 9 - protein, 39 aa, synthetic construct; identical to SEQ ID NO: 8
  SITE 2: note = D-alanine
  SITE 14: note = Nle
  MOD_RES 39: note = C-terminus is amidated

SEQ ID NO: 10 - protein, 39 aa, synthetic construct; identical to SEQ ID NO: 8
  SITE 2: note = D-alanine
  SITE 14: note = Nle
  SITE 39: note = sulfur of cysteine is PEGylated

SEQ ID NO: 11 - protein, 39 aa, synthetic construct; identical to SEQ ID NO: 8
  SITE 2: note = D-alanine
  SITE 14: note = Nle
  SITE 39: note = sulfur of cysteine is PEGylated
  MOD_RES 39: note = C-terminus is amidated

SEQ ID NO: 12 - protein, 31 aa, synthetic construct
HAEGTFTSDVSSYLEGQAAKEFIAWLVRGRG

SEQ ID NO: 13 - protein, 31 aa, synthetic construct; identical to SEQ ID NO: 12
  MOD_RES 31: note = C-terminus is amidated

SEQ ID NO: 14 - protein, 31 aa, synthetic construct
HVEGTFTSDVSSYLEEQAAREFIKWLVRGRG

SEQ ID NO: 15 - protein, 31 aa, synthetic construct; identical to SEQ ID NO: 14
  MOD_RES 31: note = C-terminus is amidated

SEQ ID NO: 16 - protein, 31 aa, synthetic construct
HGEGTFTSDVSSYLEGQAAKEFIAWLVRGRG

SEQ ID NO: 17 - protein, 31 aa, synthetic construct; identical to SEQ ID NO: 16
  MOD_RES 31: note = C-terminus is amidated

SEQ ID NO: 18 - protein, 32 aa, synthetic construct
  SITE 2: note = Aib
HXEGTFTSDVSSYLEGQAAKEFIAWLVRGRGL

SEQ ID NO: 19 - protein, 32 aa, synthetic construct; identical to SEQ ID NO: 18
  SITE 2: note = Aib
  MOD_RES 32: note = C-terminus is amidated

SEQ ID NO: 20 - protein, 39 aa, synthetic construct
  SITE 2: note = Aib
  SITE 13: note = Aib
  MOD_RES 39: note = C-terminus is amidated
YXEGTFTSDYSIXLDKIAQKAFVQWLIAGGPSSGAPPPS

SEQ ID NO: 21 - protein, 39 aa, synthetic construct; identical to SEQ ID NO: 20
  SITE 2: note = Aib
  SITE 13: note = Aib

SEQ ID NO: 22 - protein, 30 aa, synthetic construct
HSQGTFTSDKSEYLDSERARDFVAWLEAGG

SEQ ID NO: 23 - protein, 30 aa, synthetic construct; identical to SEQ ID NO: 22
  MOD_RES 30: note = C-terminus is amidated

SEQ ID NO: 24 - protein, 29 aa, synthetic construct
  SITE 2: note = 1-amino-cyclobutanecarboxylic acid (Ac4c)
  MOD_RES 29: note = C-terminus is amidated
HXQGTFTSDYSKYLDERAAKDFIKWLESA

SEQ ID NO: 25 - protein, 29 aa, synthetic construct
  SITE 2: note = 1-amino-cyclobutanecarboxylic acid (Ac4c)
HXQGTFTSDYSKYLDERAAKDFIKWLESA

SEQ ID NO: 26 - protein, 29 aa, synthetic construct
  SITE 2: note = Aib
  SITE 16: note = E at position 16 and K at position 20 are connected via a lactam bridge
  SITE 20: note = E at position 16 and K at position 20 are connected via a lactam bridge
HXQGTFTSDYSKYLDEKAAKEFIQWLLQT

SEQ ID NO: 27 - protein, 29 aa, synthetic construct
  SITE 2: note = Aib
  SITE 16: note = E at position20 and K at position 20 are connected via a lactam bridge
  SITE 20: note = E at position20 and K at position 20 are connected via a lactam bridge
  MOD_RES 29: note = C-terminus is amidated
HXQGTFTSDYSKYLDEKAAKEFIQWLLQT

SEQ ID NO: 28 - protein, 34 aa, synthetic construct
  SITE 2: note = Aib
  MOD_RES 34: note = C-terminus is amidated
HXQGTFTSDYSKYLDEKKAKEFVEWLLEGGPSSG

SEQ ID NO: 29 - protein, 34 aa, synthetic construct; identical to SEQ ID NO: 28
  SITE 2: note = Aib

SEQ ID NO: 30 - protein, 33 aa, synthetic construct
  SITE 2: note = Aib
HXEGSFTSELATILDKQAARDFIAWLIQHKITD

SEQ ID NO: 31 - protein, 33 aa, synthetic construct; identical to SEQ ID NO: 30
  SITE 2: note = Aib
  MOD_RES 33: note = C-terminus is amidated

SEQ ID NO: 32 - protein, 39 aa, synthetic construct
  SITE 2: note = Aib
  SITE 13: note = alpha-methyl-leucine (alphaMeL)
  SITE 20: note = Aib
  MOD_RES 39: note = C-terminus is amidated
YXQGTFTSDYSIXLDKKAQXAFIEYLLEGGPSSGAPPPS

SEQ ID NO: 33 - protein, 39 aa, synthetic construct; identical to SEQ ID NO: 32
  SITE 2: note = Aib
  SITE 13: note = alpha-methyl-leucine (alphaMeL)
  SITE 20: note = Aib

SEQ ID NO: 34 - protein, 18 aa, synthetic construct
WWEQDRDWDFDVFGGGTP

SEQ ID NO: 35 - protein, 11 aa, synthetic construct
  DISULFID 3..9: note = intrachain disulfide bond
DICLPRWGCLW

SEQ ID NO: 36 - protein, 18 aa, synthetic construct
  DISULFID 7..13: note = intrachain disulfide bond
RLIEDICLPRWGCLWEDD

SEQ ID NO: 37 - protein, 46 aa, synthetic construct
LAEAKVLANRELDKYGVSDFYKRLINKAKTVEGVEALKLHILAALP

SEQ ID NO: 38 - protein, 46 aa, synthetic construct
IAEAKEAANAELDSYGVSDFYKRLIDKAKTVEGVEALKDAILAALP

SEQ ID NO: 39 - protein, 8 aa, synthetic construct
  SITE 1: note = fluorescein-(AEEA), wherein AEEA is 2-(2-(2-aminoethoxy)acetyl
  SITE 5: note = K(palmitate)
XEYEXEYE

SEQ ID NO: 40 - protein, 31 aa, synthetic construct; identical to SEQ ID NO: 2
  SITE 2: note = Aib
  LIPID 20: note = ([2-(2-amino-ethoxy)-ethoxy]-acetyl)2-(gamma-Glu)-C O-(CH2)16-CO2H

SEQ ID NO: 41 - protein, 31 aa, synthetic construct; identical to SEQ ID NO: 12
  LIPID 20: note = palmitoyl-Gln-OH

SEQ ID NO: 42 - protein, 31 aa, synthetic construct; identical to SEQ ID NO: 14
  LIPID 24: note = ([2-(2-amino-ethoxy)-ethoxy]-acetyl)2-(gamma-Glu)-C O-(CH2)16-CO2H

SEQ ID NO: 43 - protein, 31 aa, synthetic construct; identical to SEQ ID NO: 16
  LIPID 20: note = ([2-(2-amino-ethoxy)-ethoxy]-acetyl)2-(gamma-Glu)-C O-(CH2)20-CO2H

SEQ ID NO: 44 - protein, 32 aa, synthetic construct; identical to SEQ ID NO: 18
  SITE 2: note = Aib
  LIPID 20: note = AEEA-Aib-gammaGlu-ODDA

SEQ ID NO: 45 - protein, 39 aa, synthetic construct; identical to SEQ ID NO: 20
  SITE 2: note = Aib
  SITE 13: note = Aib
  LIPID 20: note = ([2-(2-amino-ethoxy)-ethoxy]-acetyl)2-(gammaGlu)1-C O-(CH2)18-CO2H
  MOD_RES 39: note = C-terminus is amidated

SEQ ID NO: 46 - protein, 30 aa, synthetic construct; identical to SEQ ID NO: 22
  LIPID 10: note = gamma-Glu-palmitoyl

SEQ ID NO: 47 - protein, 29 aa, synthetic construct
  SITE 2: note = 1-amino-cyclobutanecarboxylic acid (Ac4c)
  LIPID 24: note = [l7-carboxy-heptadecanoyl]-isoGlu-GSGSGG
  MOD_RES 29: note = C-terminus is amidated
HXQGTFTSDYSKYLDERAAKDFIKWLESA

SEQ ID NO: 48 - protein, 29 aa, synthetic construct
  SITE 2: note = Aib
  SITE 16: note = E at position 16 and the K at position 20 are connected via a lactam bridge
  SITE 20: note = E at position 16 and the K at position 20 are connected via a lactam bridge
  LIPID 17: note = glucuronic acid C-18
  MOD_RES 29: note = C-terminus is amidated
HXQGTFTSDYSKYLDEKAAKEFIQWLLQT

SEQ ID NO: 49 - protein, 34 aa, synthetic construct; identical to SEQ ID NO: 28
  SITE 2: note = Aib
  LIPID 20: note = ([2-(2-amino-ethoxy)-ethoxy]-acetyl)2-(gamma-Glu)-C O-(CH2)18-CO2H
  MOD_RES 34: note = C-terminus is amidated

SEQ ID NO: 50 - protein, 33 aa, synthetic construct; identical to SEQ ID NO: 30
  SITE 2: note = Aib
  LIPID 16: note = [17-carboxy-heptadecanoyl]-isoGlu

SEQ ID NO: 51 - protein, 39 aa, synthetic construct; identical to SEQ ID NO: 32
  SITE 2: note = Aib
  SITE 13: note = alpha-methyl-leucine (alphaMeL)
  SITE 20: note = Aib
  LIPID 17: note = (2-[2-(2-amino-ethoxy)-ethoxy]-acetyl)-(gammaGlu)-C O-(CH2)18-CO2H
  MOD_RES 39: note = C-terminus is amidated

SEQ ID NO: 52 - protein, 35 aa, synthetic construct
  MOD_RES 1: note = D-Lysine, wherein the side chain is acylated with COC16H32CO2H via a gamma Glu-(MINIPEG)2-gamma Glu spacer, wherein MINIPEG is COCH2OCH2CH2OCH2CH2NH
  SITE 2: note = n-methyl glycine (sarcosine)
  MOD_RES 35: note = side chain of lysine is acylated with COC16H32CO2H via a gamma Glu-(MINIPEG)2-gamma Glu spacer, wherein MINIPEG is COCH2OCH2CH2OCH2CH2NH
KXSVSEIQLMHNLGKHLNSMERVEWLRKKLQDVHK

SEQ ID NO: 53 - protein, 35 aa, synthetic construct; identical to SEQ ID NO: 52
  MOD_RES 1: note = D-Lysine, wherein the side chain is acylated with COC16H32CO2H via a gamma Glu-(MINIPEG)2-gamma Glu spacer, wherein (miniPEG)2 is COCH2OCH2CH2OCH2CH2NH
  SITE 2: note = n-methyl glycine (sarcosine)
  MOD_RES 35: note = side chain of lysine is acylated with COC16H32CO2H via a gamma Glu-(MINIPEG)2-gamma Glu spacer, wherein (miniPEG)2 is COCH2OCH2CH2OCH2CH2NH

SEQ ID NO: 54 - protein, 40 aa, synthetic construct
  SITE 2: note = AiB
HXEGTFTSDVSSYLEEEAAKEFIAWLVRGGPSSGAPPPSK

SEQ ID NO: 55 - protein, 31 aa, synthetic construct
  SITE 2: note = Aib
HXEGTFTSDVSSYLEEQAAKEFIAWLVRGGG

SEQ ID NO: 56 - protein, 71 aa, synthetic construct
  SITE 2: note = Aib
HXEGTFTSDVSSYLEEQAAKEFIAWLVRGGGGAQPGAQPGAQPGAQPGAQPGAQPGAQPGAQPGAQPGQKP

SEQ ID NO: 57 - protein, 39 aa, synthetic construct; identical to SEQ ID NO: 20
  SITE 13: note = AiB
  SITE 2: note = 2; note = Aib

SEQ ID NO: 58 - protein, 191 aa, Homo sapiens [sequence omitted]